(19) United States
(12) Patent Application Publication
(10) Pub. No.: US 2025/0107437 A1
(43) Pub. Date: Mar. 27, 2025

(54) NITROGEN-CONTAINING COMPOUND AND ORGANIC ELECTROLUMINESCENT DEVICE AND ELECTRONIC APPARATUS

(71) Applicant: SHAANXI LIGHTE OPTOELECTRONICS MATERIAL CO. , LTD., Xi'an, Shaanxi (CN)

(72) Inventors: Xianbin Xu, Xi'an, Shaanxi (CN); Lei YANG, Xi'an, Shaanxi (CN)

(21) Appl. No.: 18/552,345

(22) PCT Filed: Mar. 13, 2023
(86) PCT No.: PCT/CN2023/081183

§ 371 Date: Sep. 25, 2023

(30) Foreign Application Priority Data
Jun. 13, 2022 (CN) 202210662921.9

(51) Int. Cl. H10K 85/60; C07D 498/04; C07D 515/04; H10K 50/12

(52) U.S. Cl. CPC H10K 85/657; C07D 498/04; C07D 515/04; H10K 85/615; H10K 85/622; H10K 85/624; H10K 85/626; H10K 85/654; H10K 85/6572; H10K 85/6574; H10K 85/6576; H10K 50/12

(57) ABSTRACT

The present application relates to the technical field of organic electroluminescent materials, and provides a nitrogen-containing compound, an organic electroluminescent device, and an electronic apparatus comprising the nitrogen-containing compound. The nitrogen-containing compound comprises a core structure that is a naphthafurooxazolyl/thiazolyl group. The nitrogen-containing compound, when used as the host material of an light emitting layer of an organic electroluminescent device, can significantly improve the luminescence efficiency and the service life of the device.

### CROSS-REFERENCE TO RELATED APPLICATIONS

The present application is a U.S. National Stage Application of International Application no. PCT/CN2023/081183, filed on Mar. 31, 2023, which claims the priority of Chinese patent application No. 202210662921.9 filed on Jun. 13, 2022, both of which are incorporated herein by reference in their entireties as a part of this application.

### FIELD OF THE INVENTION

The present application relates to the technical field of organic electroluminescent materials, and in particular to a nitrogen-containing compound, an organic electroluminescent device and an electronic apparatus comprising the nitrogen-containing compound.

### BACKGROUND OF THE INVENTION

With the development of electronic technology and the advancement of material science, electronic devices used to achieve electroluminescence or photoelectric conversion have found an increasingly wide range of applications. An organic electroluminescent device (OLED) typically comprises a cathode and an anode disposed opposite to each other, and a functional layer disposed between the cathode and the anode. The functional layer consists of a plurality of organic or inorganic film layers, and generally comprises an organic light emitting layer, a hole transport layer, an electron transport layer, etc. When a voltage is applied to the cathode and the anode, an electric field is formed between the two electrodes. Under the influence of the electric field, electrons on the cathode side migrate to the electroluminescent light emitting layer, and holes on the anode side also migrate to the light emitting layer. The electrons and the holes recombine in the electroluminescent light emitting layer, forming excitons. The excitons in excited states release energy, causing the electroluminescent light emitting layer to emit light to the outside.

Main problems with existing organic electroluminescent devices lie in their service life and efficiency. As display becomes larger and larger, driving voltage is increased accordingly, which necessitates increases in luminescence efficiency and current efficiency. It is therefore necessary to continue to develop a new material to further improve the performance of organic electroluminescent device.

### SUMMARY OF THE INVENTION

Directed against the above problems with the existing technology, the present application aims at providing a nitrogen-containing compound, an electronic device and an electronic apparatus comprising the nitrogen-containing compound. The nitrogen-containing compound, when used in an organic electroluminescent device, can improve the performance of the device.

A first aspect of the present application provides a nitrogen-containing compound having a structure as shown in Formula 1:

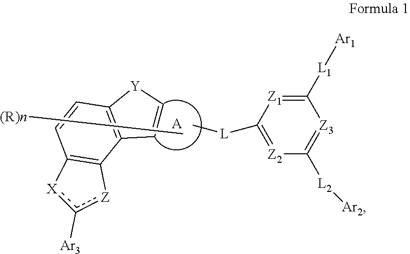

wherein, Y is selected from S or O;
one of X and Z is —N═, and the other is O or S;
Z₁, Z₂, and Z₃ are selected from C(R₁) or N, and at least two of Z₁, Z₂, and Z₃ are N;
Ring A is selected from a naphthalene ring or a phenanthrene ring;
L, L₁, and L₂ are identical or different, and are each independently selected from a single bond, a substituted or unsubstituted arylene having 6 to 30 carbon atoms, or a substituted or unsubstituted heteroarylene having 3 to 30 carbon atoms;
Ar₁, Ar₂, and Ar₃ are identical or different, and are each independently selected from a substituted or unsubstituted aryl having 6 to 40 carbon atoms, a substituted or unsubstituted heteroaryl having 3 to 40 carbon atoms, an alkyl having 1 to 10 carbon atoms, or a cycloalkyl having 3 to 10 carbon atoms;
substituents of L, L₁, L₂, Ar₁, Ar₂, and Ar₃ are identical or different, and are each independently selected from deuterium, a cyano, a halogen, an alkyl having 1 to 10 carbon atoms, a haloalkyl having 1 to 10 carbon atoms, a deuterated alkyl having 1 to 10 carbon atoms, a trialkylsiyl having 3 to 12 carbon atoms, a triphenylsilyl, an aryl having 6 to 20 carbon atoms, a deuterated aryl having 6 to 20 carbon atoms, a heteroaryl having 3 to 20 carbon atoms, a cycloalkyl having 3 to 10 carbon atoms, an alkoxy having 1 to 10 carbon atoms, an alkylthio having 1 to 10 carbon atoms, an aryloxy having 6 to 20 carbon atoms, or an arylthio having 6 to 20 carbon atoms; optionally, any two adjacent substituents form a saturated or unsaturated 3-membered to 15-membered ring;
each R₁ and each R are identical or different, and are independently selected from hydrogen, deuterium, a cyano, a halogen, an alkyl having 1 to 10 carbon atoms, a haloalkyl having 1 to 10 carbon atoms, a deuterated alkyl having 1 to 10 carbon atoms, a trialkylsiyl having 3 to 12 carbon atoms, a triphenylsilyl, an aryl having 6 to 20 carbon atoms, a heteroaryl having 3 to 20 carbon atoms, or a cycloalkyl having 3 to 10 carbon atoms; optionally, any two adjacent R₁s or any two adjacent Rs form a ring; n is selected from 1, 2, 3, 4, 5, 6, 7, 8, or 9.

A second aspect of the present application provides an organic electroluminescent device comprising an anode and a cathode disposed opposite to each other, and a functional layer disposed between the anode and the cathode. The functional layer comprises the nitrogen-containing compound described above.

A third aspect of the present application provides an electronic apparatus including the organic electroluminescent device described in the second aspect.

The structure of the compound of the present application comprises naphtha (or phenanthro)furooxazolyl/thiazoly and a electron-deficient heteroaryl such as triazinyl and pyrimidyl. The naphtha (or phenanthro)furyl group and the oxazolyl/thiazolyl group both have electron transport properties; and after these two groups are fused together, the conjugated system is increased, which enhances the electron transport performance of the groups. The triazinyl or pyrimidyl group has an excellent electron transport property. Linking of the naphtha (or phenanthro)furooxazolyl/thiazolyl group with the electron-deficient heteroaryl group such as triazinyl and pyrimidyl gives the compound of the present application an excellent electron transport property. The compound of the present application, when mixed with a hole transport material to form a mixed-type host material, can improve the balance of charge carriers in a light emitting layer, expand a region where the carriers recombine, improve the generation and utilization efficiency of excitons as well as the luminescence efficiency and the service life of a device.

### BRIEF DESCRIPTION OF THE DRAWINGS

The accompanying drawings are intended to provide a further understanding of the present application and form a part of the specification. The accompanying drawings, together with the following specific embodiments, are used to illustrate the present application, but do not constitute any limitation to the present application.

FIG. 1 is a schematic structural diagram of an organic electroluminescent device according to an embodiment of the present application.

FIG. 2 is a schematic structural diagram of an electronic apparatus according to an embodiment of the present application.

### LIST OF REFERENCE SIGNS

100: anode
200: cathode
300: functional layer
310: hole injection layer
321: first hole transport layer
322: second hole transport layer
330: organic light emitting layer
340: electron transport layer
350: electron injection layer
400: electronic apparatus

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided to make the present application more comprehensive and complete, and to communicate the concepts of these exemplary embodiments fully to those skilled in the art. Features, structures, or characteristics described can be combined in one or more embodiments in any suitable manner. In the following description, many specific details are provided to give a full understanding of the embodiments of the present application.

In a first aspect, the present application provides a nitrogen-containing compound having a structure as shown in Formula 1:

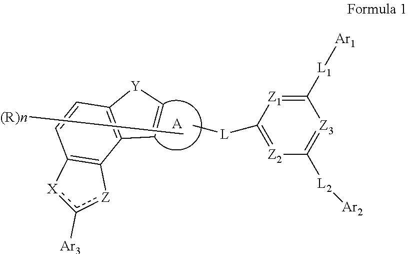

wherein, Y is selected from S or O;
one of X and Z is —N═, and the other is O or S;
Z₁, Z₂, and Z₃ are selected from C(R₁) or N, and at least two of Z₁, Z₂, and Z₃ are N;
Ring A is selected from a naphthalene ring or a phenanthrene ring;
L, L₁, and L₂ are identical or different, and are each independently selected from a single bond, a substituted or unsubstituted arylene having 6 to 30 carbon atoms, or a substituted or unsubstituted heteroarylene having 3 to 30 carbon atoms;
Ar₁, Ar₂, and Ar₃ are identical or different, and are each independently selected from a substituted or unsubstituted aryl having 6 to 40 carbon atoms, a substituted or unsubstituted heteroaryl having 3 to 40 carbon atoms, an alkyl having 1 to 10 carbon atoms, or a cycloalkyl having 3 to 10 carbon atoms;
substituents of L, L₁, L₂, Ar₁, Ar₂, and Ar₃ are identical or different, and are each independently selected from deuterium, a cyano, a halogen, an alkyl having 1 to 10 carbon atoms, a haloalkyl having 1 to 10 carbon atoms, a deuterated alkyl having 1 to 10 carbon atoms, a trialkylsiyl having 3 to 12 carbon atoms, a triphenylsilyl, an aryl having 6 to 20 carbon atoms, a deuterated aryl having 6 to 20 carbon atoms, a heteroaryl having 3 to 20 carbon atoms, a cycloalkyl having 3 to 10 carbon atoms, an alkoxy having 1 to 10 carbon atoms, an alkylthio having 1 to 10 carbon atoms, an aryloxy having 6 to 20 carbon atoms, or an arylthio having 6 to 20 carbon atoms; optionally, any two adjacent substituents form a saturated or unsaturated 3-membered to 15-membered ring;
each R₁ and each R are identical or different, and are each independently selected from hydrogen, deuterium, a cyano, a halogen, an alkyl having 1 to 10 carbon atoms, a haloalkyl having 1 to 10 carbon atoms, a deuterated alkyl having 1 to 10 carbon atoms, a trialkylsiyl having 3 to 12 carbon atoms, a triphenylsilyl, an aryl having 6 to 20 carbon atoms, a heteroaryl having 3 to 20 carbon atoms, or a cycloalkyl having 3 to 10 carbon atoms; optionally, any two adjacent R₁s or any two adjacent Rs form a ring; n is selected from 1, 2, 3, 4, 5, 6, 7, 8, or 9.

In the present application, the term “optional” or “optionally” means that the subsequently described event or circumstance may or may not occur. As an example, “optionally, any two adjacent substituents form a ring” means that the two substituents may or may not form a ring, i.e., including instances where the two adjacent substituents form a ring and instances where the two adjacent substituents do not form a ring. As another example, “optionally, in Ar₁, Ar₂, and Ar₃, any two adjacent substituents form a ring” means that any two adjacent substituents of Ar₁, Ar₂, and Ar₃, are linked to each other to form a ring, or any two adjacent substituents of Ar₁, Ar₂, and Ar₃ may exist independently. The mentioning of “any two adjacent” may involve instances where there are two substituents on a same atom and also instances where there is one substituent on each of two adjacent atoms. When there are two substituents on a same atom, the two substituents, together with the atom to which they are attached, may form a saturated or unsaturated spiro ring; and when there is one substituent on each of two adjacent atoms, the two substituents may be fused into a ring.

In the present application, the expression “each . . . independently” may be used interchangeably with the expressions “ . . . respectively and independently”, and “ . . . each independently”, and all these expressions should be interpreted in a broad sense. They can not only mean that, in different groups, specific options expressed between the same symbols do not affect each other, but also mean that in a same group, specific options expressed between the same symbols do not affect each other.

For example, the meaning of

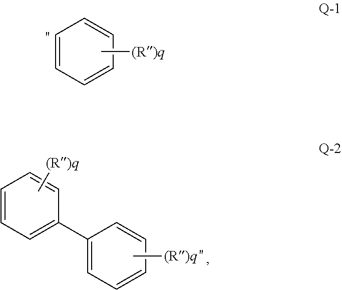

where each q is independently 0, 1, 2 or 3, and each R″ is independently selected from hydrogen, deuterium, fluorine or chlorine″ is as follows: Formula Q-1 represents that q substituent R″s exist on a benzene ring, each R″ can be the identical or different, and options of each R″ do not affect each other; and Formula Q-2 represents that each benzene ring of biphenyl has q substituent R″s, the number q of the substituent R″s on the two benzene rings can be the identical or different, each R″ can be the identical or different, and options of each R″ do not affect each other.

In the present application, the term “substituted or unsubstituted” means that the functional group defined by the term may or may not have a substituent (hereinafter referred to as Rc for ease of description). For example, “substituted or unsubstituted aryl” refers to an aryl group with a substituent Rc or an aryl group without a substituent. The above substituents, namely Rc, may be, for example, deuterium, halogen, cyano, heteroaryl, aryl, trialkylsilyl, alkyl, haloalkyl, cycloalkyl, etc. The number of the substitutes may be one or more.

In the present application, “more” means more than 2, such as 2, 3, 4, 5, or 6, etc.

In the present application, the term “and/or” is used to connect two elements, and it indicates that the two elements both appear or either of the two elements appears.

Hydrogen atoms in the structure of the compound of the present application include various isotopic atoms of hydrogen element, such as hydrogen (H), deuterium (D), or tritium (T).

In the present application, the number of carbon atoms of a substituted or unsubstituted functional group is the total number of all carbon atoms. For example, if L₁ is a substituted arylene having 12 carbon atoms, then the total number of all carbon atoms of the arylene group and substituents thereof is 12.

In the present application, when it is mentioned that a ring, such as a saturated or unsaturated 3-membered to 15-membered ring, is formed, such ring include saturated carbon ring, saturated heterocyclic ring, partially unsaturated carbon ring, partially unsaturated heterocyclic ring, aromatic carbon ring, and aromatic heterocyclic ring.

When a ring is prefixed with n-membered, n is an integer, indicating that the number of ring atoms of the ring is n. For example, a 3-membered to 15-membered ring represents a ring having 3 to 15 ring atoms, i.e., a ring having 3, 4, 5, 6, 7, 8, 9, 10, 11, 12, 13, 14, or 15 ring atoms.

In the present application, aryl refers to any functional group or substituent derived from an aromatic carbon ring. An aryl group may be a monocyclic aryl (e.g., phenyl) or a polycyclic aryl. In other words, an aryl may be a monocyclic aryl, a fused aryl, two or more monocyclic aryls linked by carbon-carbon bond, a monocyclic aryl and a fused aryl linked by carbon-carbon bond, or two or more fused aryls linked by carbon-carbon bond. That is, unless otherwise specified, two or more aromatic groups linked by carbon-carbon bond conjugation may also be regarded as an aryl in the present application. Among them, fused aryl may include, for example, bicyclic fused aryl (e.g., naphthyl), tricyclic fused aryl (e.g., phenanthryl, fluorenyl, anthryl) and the like. An aryl does not contain heteroatoms such as B, N, O, S, P, Se, Si, etc. Examples of aryl may include, but are not limited to, phenyl, naphthyl, fluorenyl, spirodifluorenyl, anthryl, phenanthryl, biphenyl, terphenyl, triphenylene

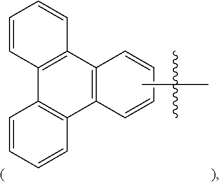

perylenyl, benzo[9,10]phenanthryl, pyrenyl, benzofluoranthenyl, chrysenyl, etc.

In the present application, arylene refers to a divalent or polyvalent group formed by further removing one or more hydrogen atoms from an aryl.

In the present application, terphenyl includes

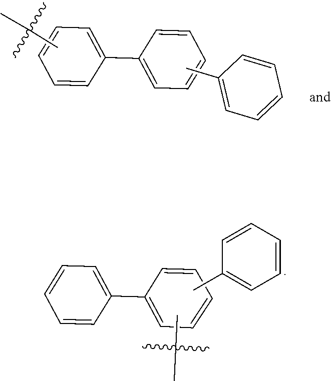

In the present application, the number of carbon atoms of substituted aryl is the total number of all carbon atoms of the aryl and substituents on the aryl. For example, substituted aryl having 18 carbon atoms means that the total number of all carbon atoms of the aryl and substituents thereof is 18.

In the present application, the number of carbon atoms of substituted or unsubstituted aryl (arylene) may be 6, 8, 10, 12, 13, 14, 15, 16, 17, 18, 19, 20, 21, 22, 23, 24, 25, 26, 28, 30, 31, 33, 34, 35, 36, 38, or 40, etc. In some embodiments, the substituted or unsubstituted aryl is a substituted or unsubstituted aryl having 6 to 40 carbon atoms. In other embodiments, the substituted or unsubstituted aryl is a substituted or unsubstituted aryl having 6 to 30 carbon atoms. In other embodiments, the substituted or unsubstituted aryl is a substituted or unsubstituted aryl having 6 to 25 carbon atoms. In other embodiments, the substituted or unsubstituted aryl is a substituted or unsubstituted aryl having 6 to 15 carbon atoms.

In the present application, fluorenyl may be substituted by one or more substituents. In the case where the above fluorenyl is substituted, the substituted fluorenyl may be, but is not limited to,

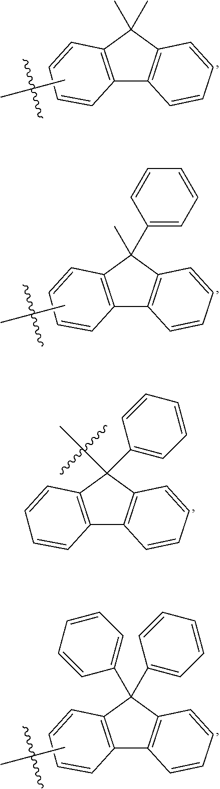

and the like.

In the present application, aryl, as a substituent of L, L₁, L₂, Ar₁, Ar₂, and Ar₃, may be, but is not limited to, phenyl, naphthyl, phenanthryl, biphenyl, fluorenyl, dimethylfluorenyl, etc.

In the present application, heteroaryl refers to a monovalent aromatic ring containing 1, 2, 3, 4, 5, or 6 heteroatoms or a derivative thereof. The heteroatoms may be one or more selected from B, O, N, P, Si, Se, and S. A heteroaryl may be a monocyclic heteroaryl or polycyclic heteroaryl. In other words, a heteroaryl may be a single aromatic ring system, or a plurality of aromatic ring systems linked by carbon-carbon bond conjugation, with any one of the aromatic ring systems being an aromatic monocyclic ring or a fused aromatic ring. For example, heteroaryl may include, but are not limited to, thiophenyl, furyl, pyrryl, imidazolyl, thiazolyl, oxazolyl, oxadiazolyl, triazolyl, pyridinyl, bipyridinyl, pyrimidyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothiophenyl, dibenzothiophenyl, thienothiophenyl, benzofuranyl, phenanthrolinyl, isoxazolinyl, thiadiazolyl, phenothiazinyl, silafluorenyl, dibenzofuranyl, N-phenylcarbazolyl, N-pyridylcarbazolyl, N-methylcarbazolyl, etc.

In the present application, heteroarylene is a divalent or polyvalent group formed by further removing one or more hydrogen atoms from a heteroaryl.

In the present application, the number of carbon atoms of substituted or unsubstituted heteroaryl (heteroarylene) may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In some embodiments, the substituted or unsubstituted heteroaryl is a substituted or unsubstituted heteroaryl having 3 to 40 carbon atoms. In other embodiments, the substituted or unsubstituted heteroaryl is a substituted or unsubstituted heteroaryl having 3 to 30 carbon atoms. In other embodiments, the substituted or unsubstituted heteroaryl is a substituted or unsubstituted heteroaryl having 5 to 12 carbon atoms.

In the present application, heteroaryl, as a substituent of L, L₁, L₂, Ar₁, Ar₂, and Ar₃, may be, but is not limited to, for example, pyridyl, carbazolyl, quinolyl, isoquinolyl, phenanthrolinyl, benzoxazolyl, benzothiazolyl, benzimidazolyl, dibenzothiophenyl, or dibenzofuranyl.

In the present application, substituted heteroaryl may mean that one or more than two hydrogen atoms in the heteroaryl are substituted by a group such as deuterium atom, halogen, cyano, aryl, heteroaryl, trialkylsilyl, alkyl, cycloalkyl, haloalkyl, etc. It should be appreciated that the number of carbon atoms of substituted heteroaryl is the total number of all carbon atoms of the heteroaryl and substituents of the heteroaryl.

In the present application, alkyl having 1 to 10 carbon atoms may include straight-chain alkyl having 1 to 10 carbon atoms and branched-chain alkyl having 3 to 10 carbon atoms. For example, the number of carbon atoms of alkyl may be, for example, 1, 2, 3, 4, 5, 6, 7, 8, 9, or 10. Specific examples of alkyl include, but are not limited to, methyl, ethyl, n-propyl, isopropyl, n-butyl, isobutyl, tert-butyl, n-pentyl, isopentyl, neopentyl, n-hexyl, etc.

In the present application, halogen may be, for example, fluorine, chlorine, bromine, or iodine.

In the present application, specific examples of trialkylsilyl include, but are not limited to, trimethylsilyl, triethylsilyl, etc.

In the present application, haloalkyl is alkyl having one or more halogen substituents. Examples of haloalkyl include, but are not limited to, trifluoromethyl.

In the present application, the number of cycloalkyl having 3 to 10 carbon atoms may be, for example, 3, 4, 5, 6, 7, 8, or 10. Specific examples of cycloalkyl include, but are not limited to, cyclopentyl, cyclohexyl, adamantyl, etc.

In the present application, a non-orientating linkage bond is single bond [symbol] extending from a ring system, and it indicates that the linkage bond can be linked at one end thereof to any position in the ring system through which the bond passes, and linked at the other end thereof to the rest of the compound molecule. For example, as shown in Formula (f) below, the naphthalyl represented by Formula (f) is linked to other positions of the molecule via two non-orientating linkage bonds passing through the two rings, which indicates any of possible linkages shown in Formulae (f-1) to (f-10):

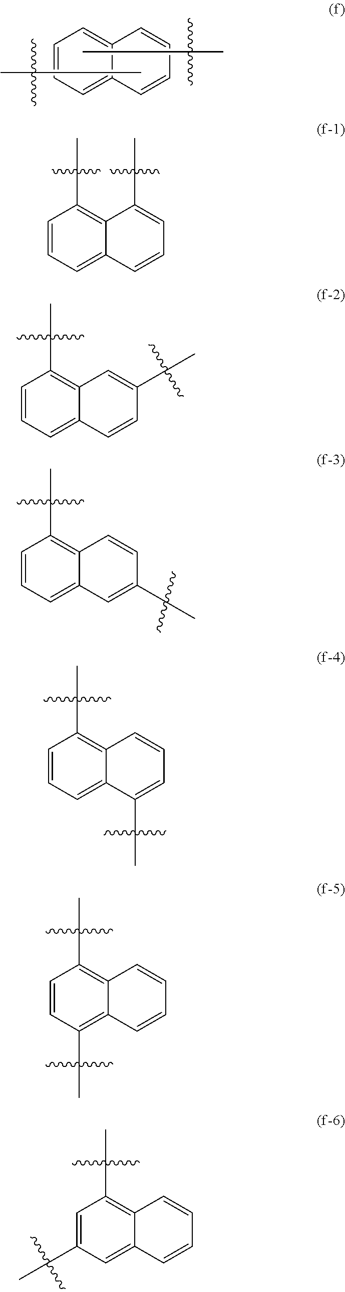

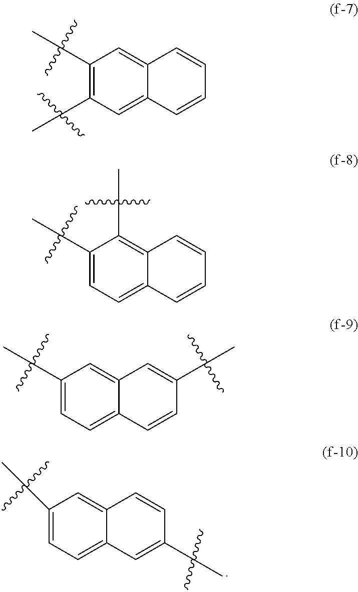 [SMILES: *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, dibenzofuranyl represented by Formula (X′) is linked to other positions of the molecule via a non-orientating linkage bond extending from the middle of a side benzene ring, which indicates any of possible linkages shown in Formulae (X′-1) to (X′-4):

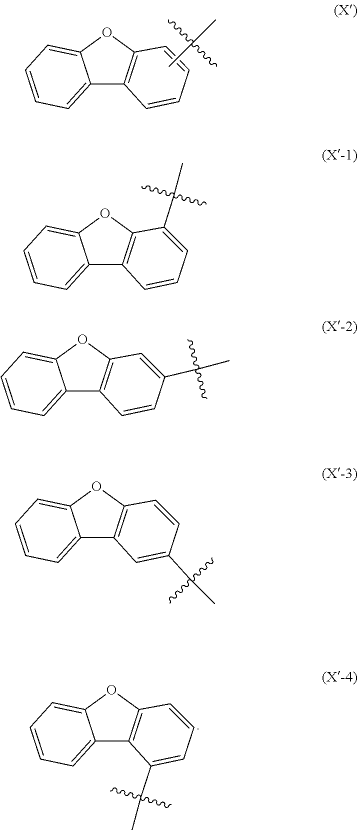

A non-orientating substituent of the present application refers to a substituent linked via single bond extending from the center of a ring system, and it means that the substituent may be linked to any possible position in the ring system. For example, as shown in Formula (Y) below, the substituent R′ represented by Formula (Y) is linked to a quinoline ring via a non-orientating linkage bond, which indicates any of possible linkages shown in Formulae (Y-1) to (Y-7):

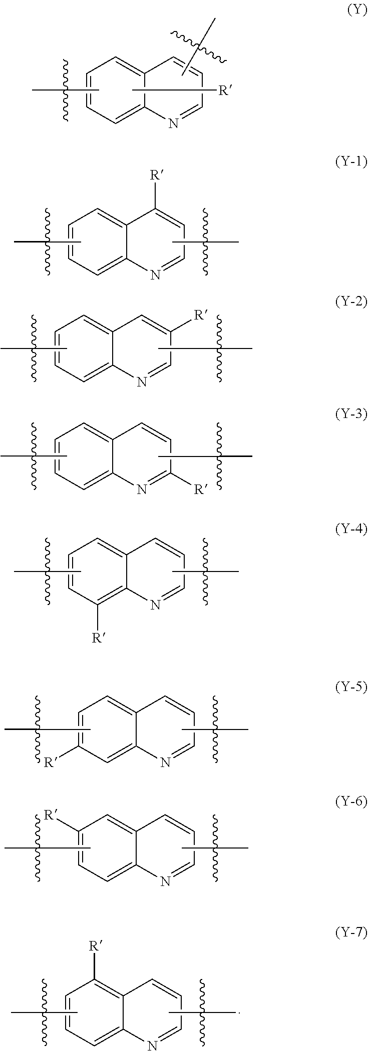

In some embodiments, the compound shown in Formula 1 is selected from structures shown in the following Formulae (1-1) to (1-16):

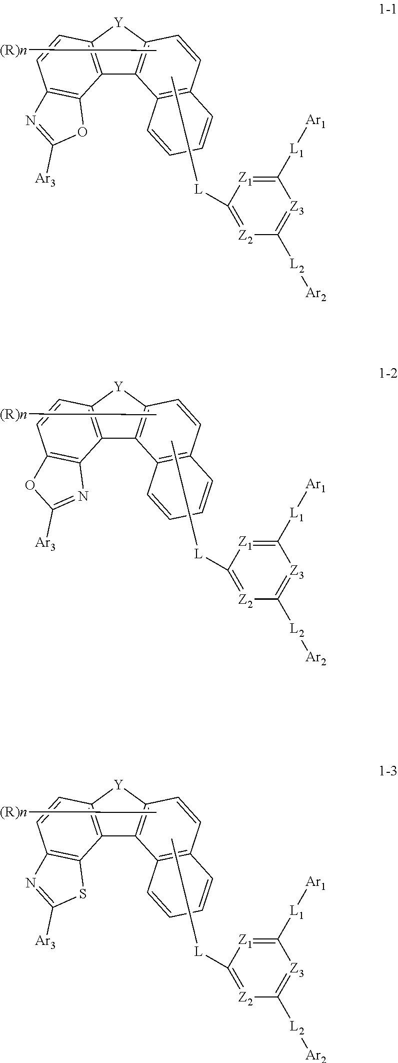

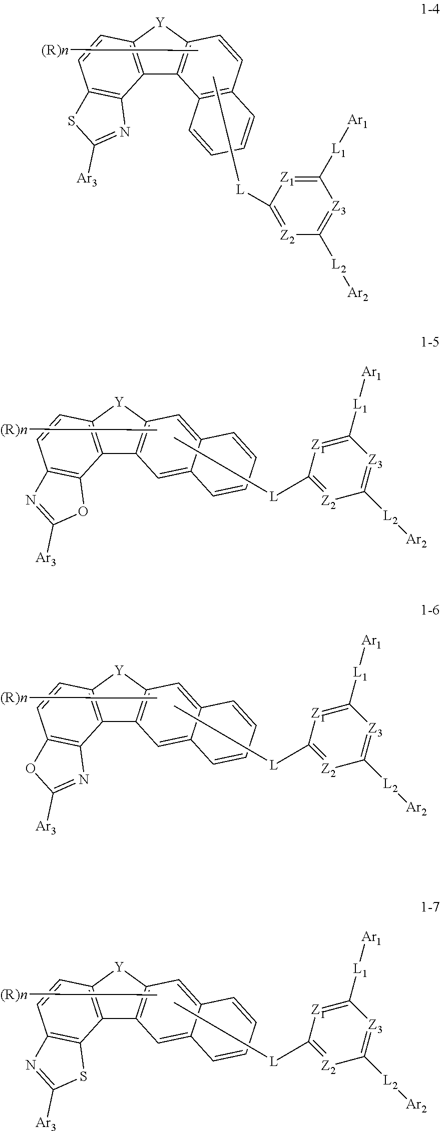

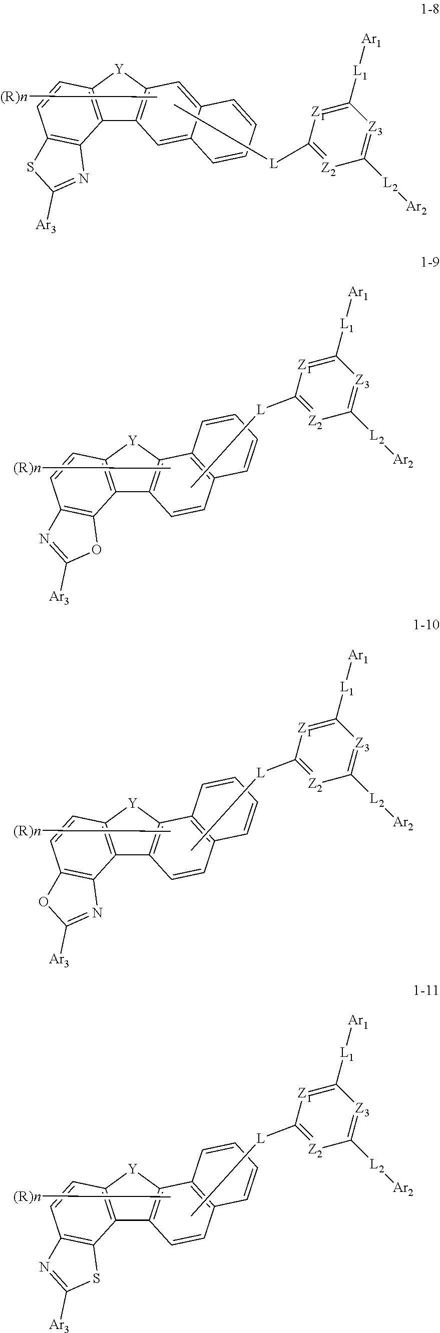

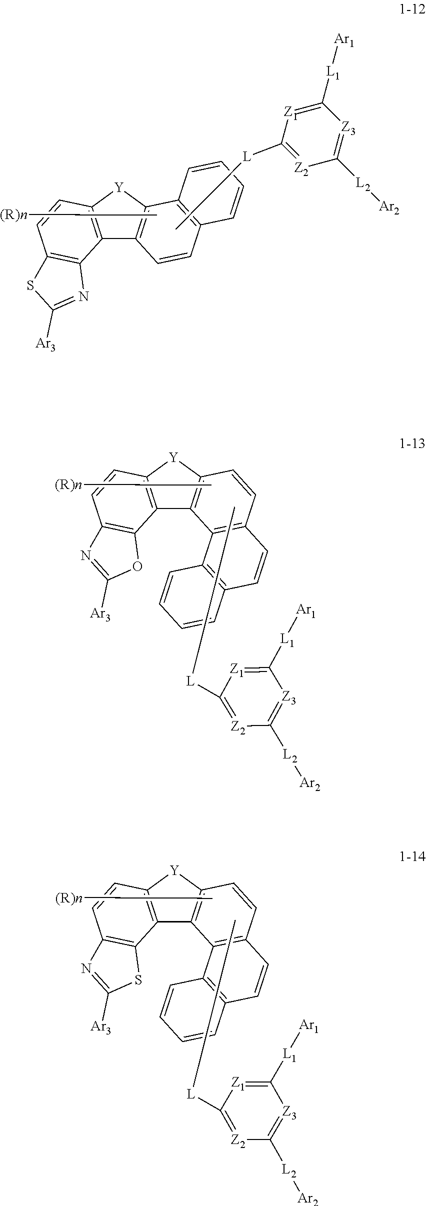

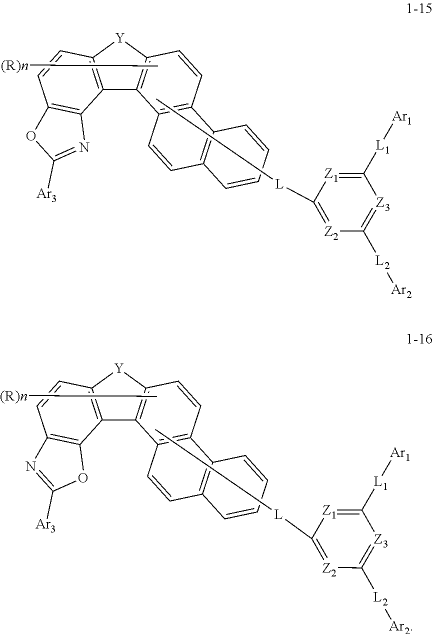

In some embodiments, the compound shown in Formula 1 is selected from structures shown in the following Formulae (2-1) to (2-11):

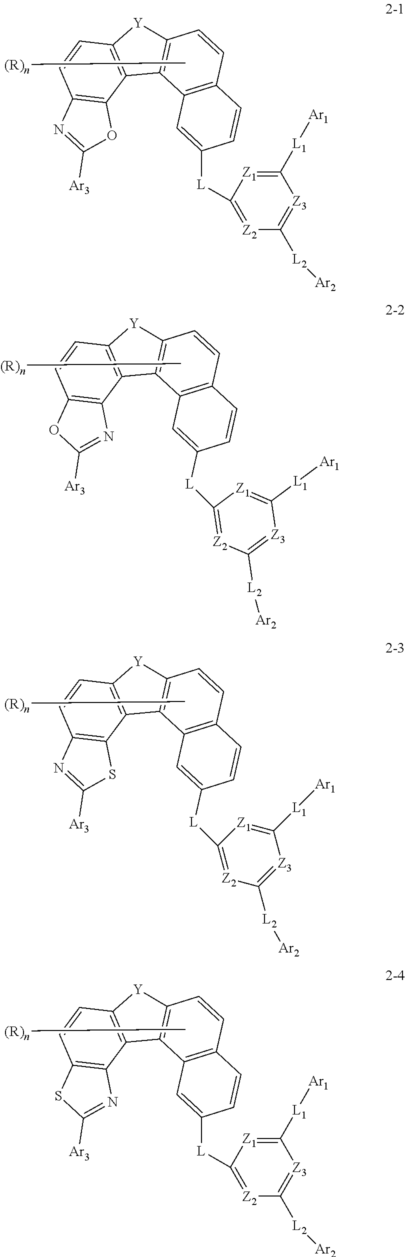

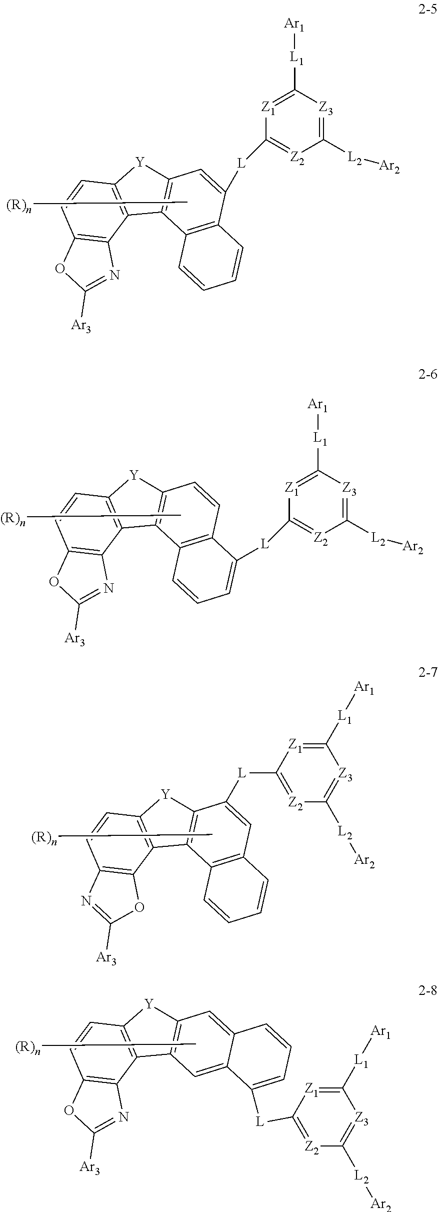

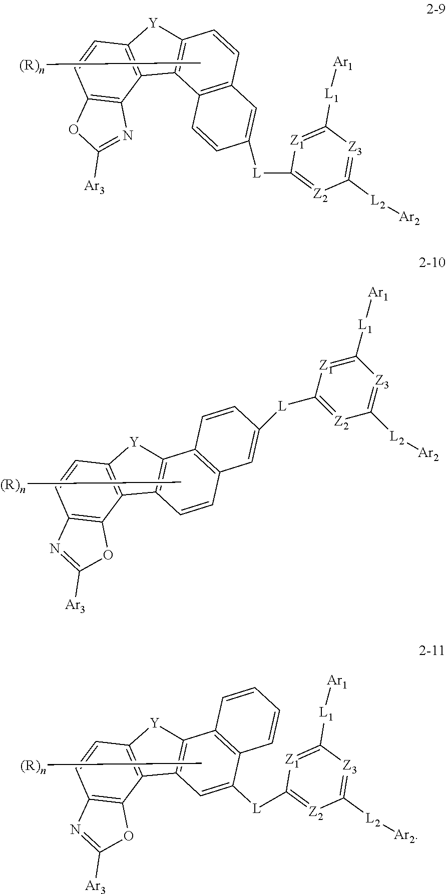

In some embodiments, Z₁ and Z₃ are N, and Z₂ is selected from C(H) or N; or, Z₁ and Z₂ are N, and Z₃ is selected from C(H) or N; or, Z₁, Z₂, and Z₃ are all N.

In some embodiments, Ar₁, Ar₂, and Ar₃ are each independently selected from a substituted or unsubstituted aryl having 6, 7, 8, 9, 10, 11, 12, 13, 14, 15, 16, 17, 18, 19, 20, 21, 22, 23, 24, or 25 carbon atoms; or a substituted or unsubstituted heteroaryl having 5, 6, 7, 8, 9, 10, 12, 13, 14, 15, 16, 17, 18, 19, 20, 21, 22, 23, 24, or 25 carbon atoms.

In some embodiments, Ar₁, Ar₂, and Ar₃ are identical or different, and are each independently selected from a substituted or unsubstituted aryl having 6 to 25 carbon atoms, or a substituted or unsubstituted heteroaryl having 5 to 24 carbon atoms.

In some embodiments, the substituents of Ar₁, Ar₂, and Ar₃ are each independently selected from deuterium, a halogen, a cyano, a haloalkyl having 1 to 4 carbon atoms, a deuterated alkyl having 1 to 4 carbon atoms, an alkyl having 1 to 4 carbon atoms, a cycloalkyl having 5 to 10 carbon atoms, an aryl having 6 to 15 carbon atoms, a heteroaryl having 5 to 12 carbon atoms, a trialkylsilyl having 3 to 8 carbon atoms, or a deuterated aryl having 6 to 15 carbon atoms; optionally, any two adjacent substituents form a benzene ring or a fluorene ring.

In some embodiments, Ar₁, Ar₂, and Ar₃ are each independently selected from a substituted or unsubstituted phenyl, a substituted or unsubstituted biphenyl, a substituted or unsubstituted terphenyl, a substituted or unsubstituted naphthyl, a substituted or unsubstituted anthryl, a substituted or unsubstituted phenanthryl, a substituted or unsubstituted fluorenyl, a substituted or unsubstituted spirobifluorenyl, a substituted or unsubstituted triphenylene, a substituted or unsubstituted pyrenyl, a substituted or unsubstituted perylenyl, a substituted or unsubstituted pyridyl, a substituted or unsubstituted dibenzothiophenyl, a substituted or unsubstituted dibenzofuranyl, a substituted or unsubstituted carbazolyl, a substituted or unsubstituted quinolyl, a substituted or unsubstituted phenanthrolinyl, a substituted or unsubstituted benzothiazolyl, a substituted or unsubstituted benzoxazolyl, or a substituted or unsubstituted benzimidazolyl.

Optionally, the substituents of Ar₁, Ar₂, and Ar₃ are each independently selected from deuterium, a fluorine, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, a dibenzofuranyl, a dibenzothiophenyl, or a carbazolyl; optionally, in Ar₁ and Ar₂, any two adjacent substituents form a benzene ring or a fluorene ring.

In some embodiments, Ar₁, Ar₂, and Ar₃ are each independently selected from a substituted or unsubstituted group W. The unsubstituted group W is selected from the group consisting of the following groups:

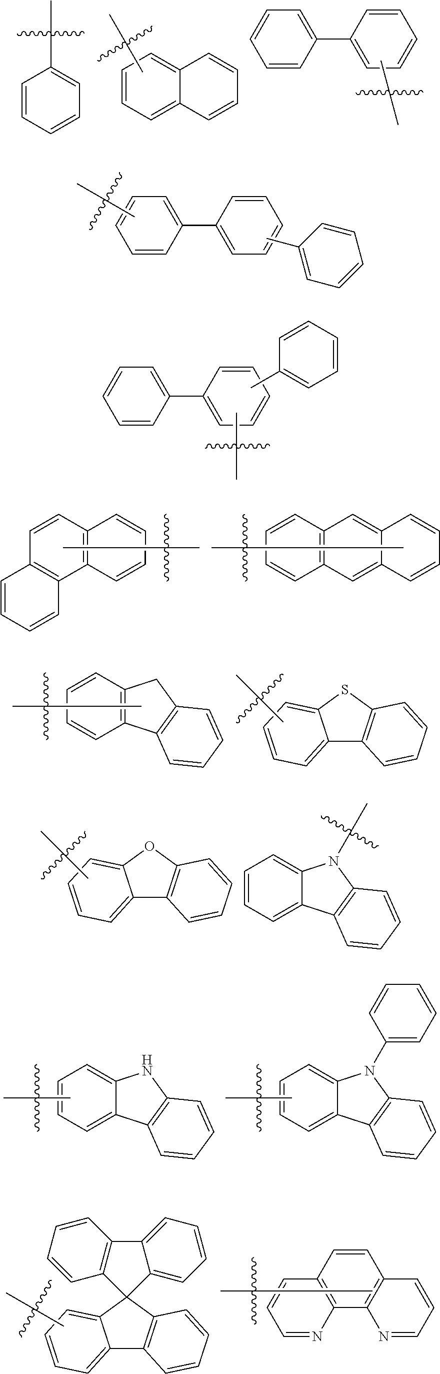

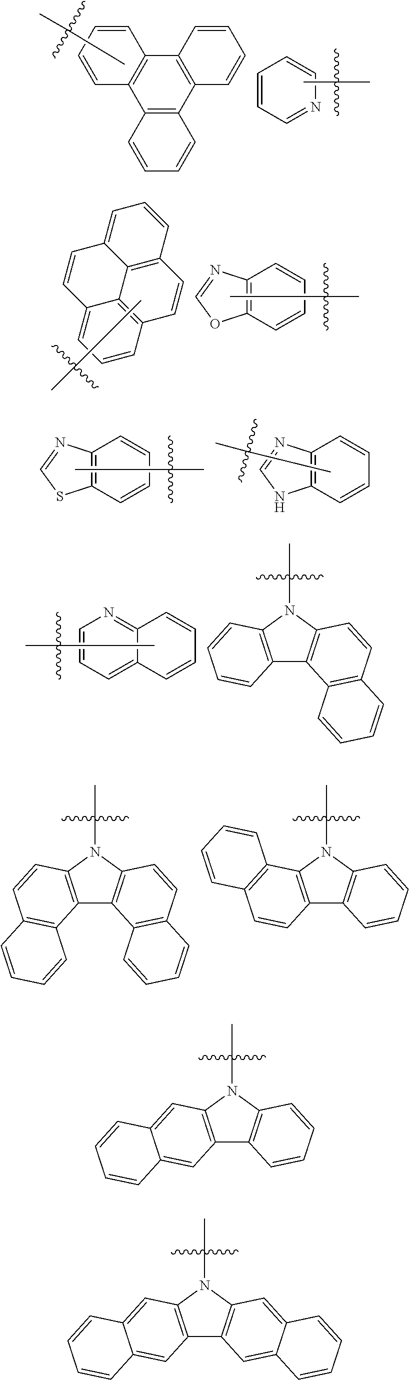

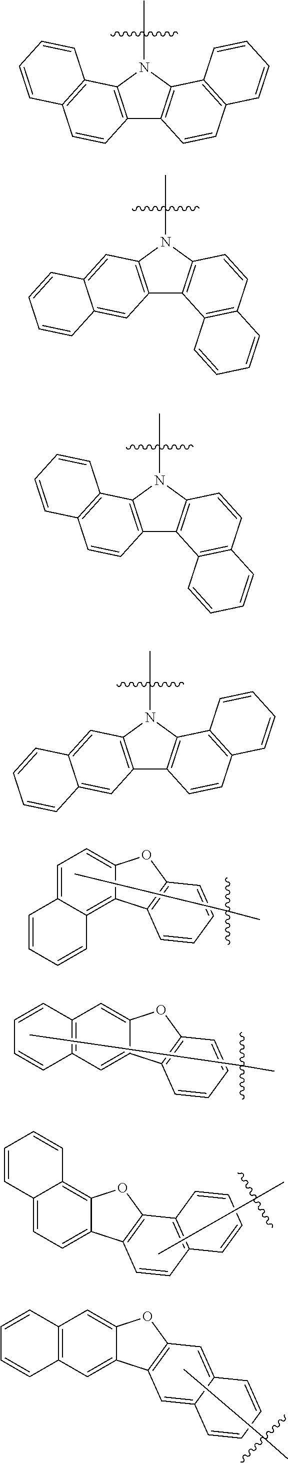

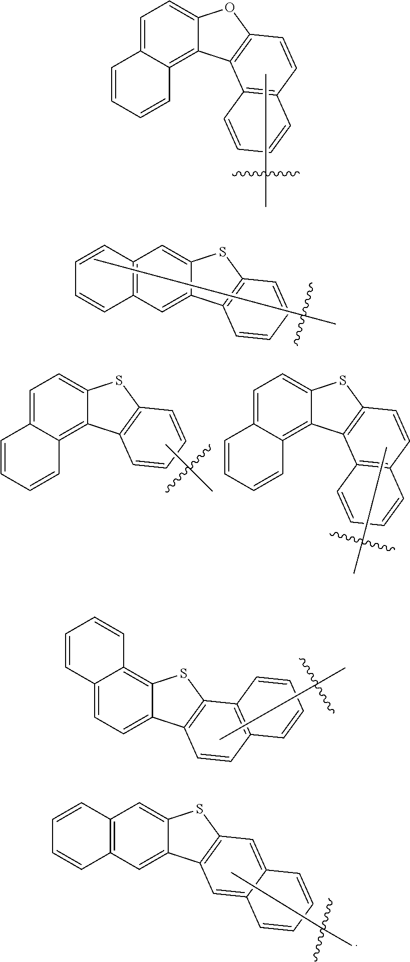

The substituted group W is the group formed by a unsubstituted group W being substituted by one or more substituents, the substituents of the substituted group W being each independently selected from deuterium, a fluorine, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, a dibenzofuranyl, a dibenzothiophenyl, a carbazolyl, a benzoxazolyl, or a benzothiazolyl; when the number of the substituents of the group W is greater than 1, the substituents are identical or different.

In some embodiments, Ar₁ and Ar₂ are respectively and independently selected from the group consisting of the following groups:

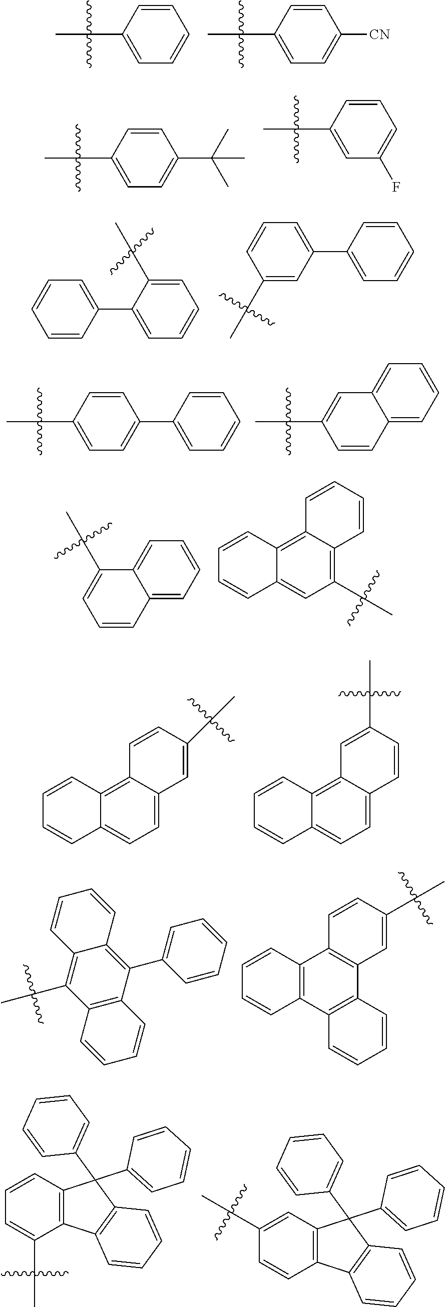

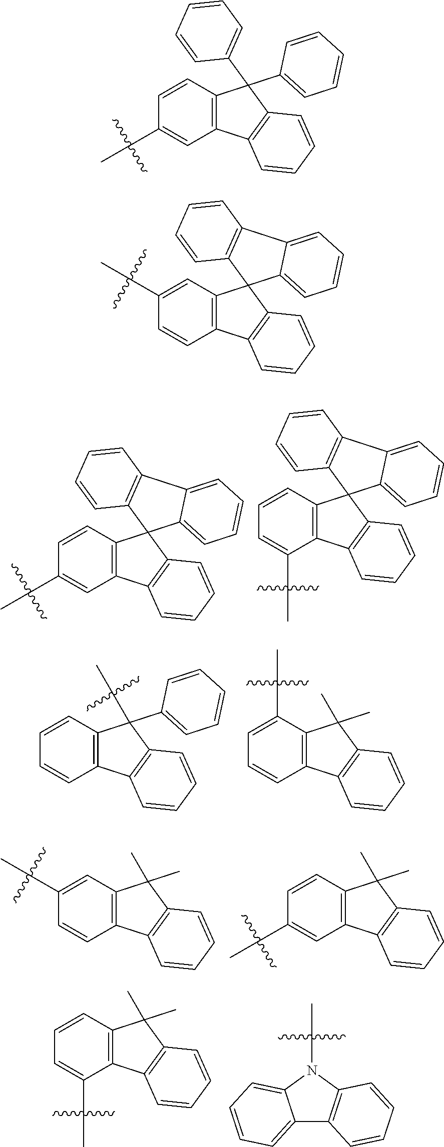

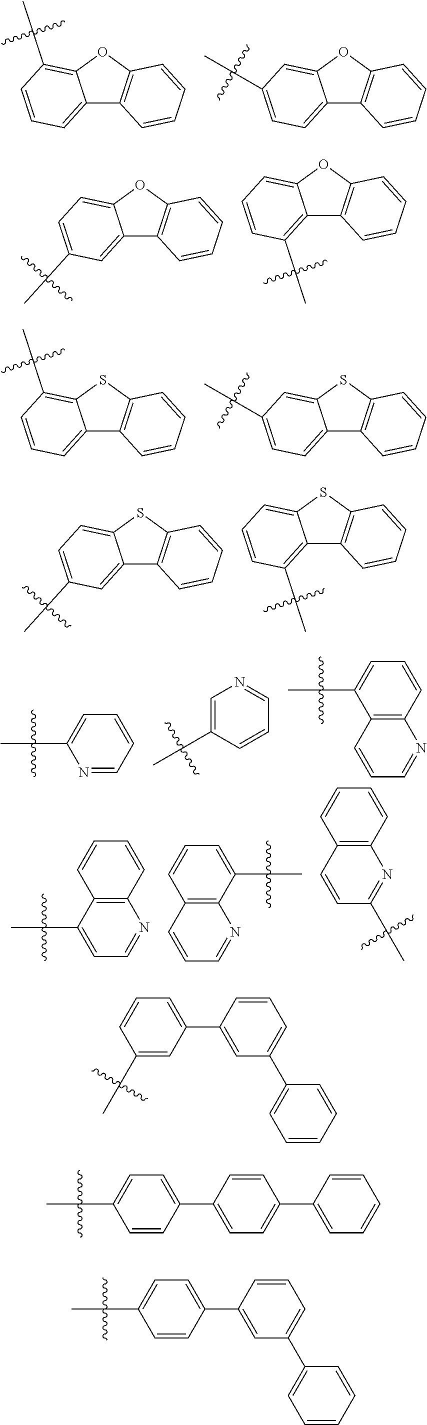

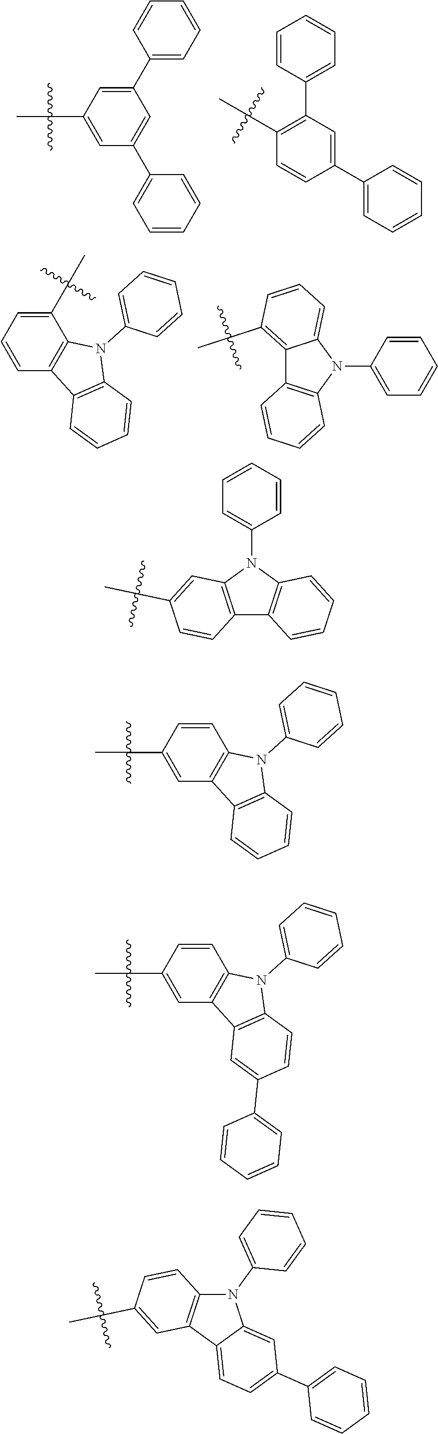

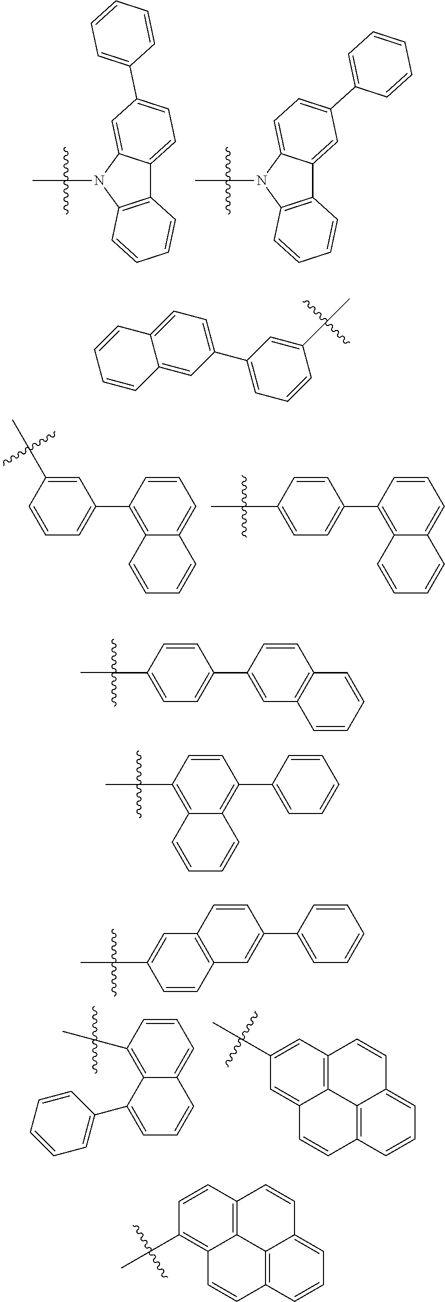

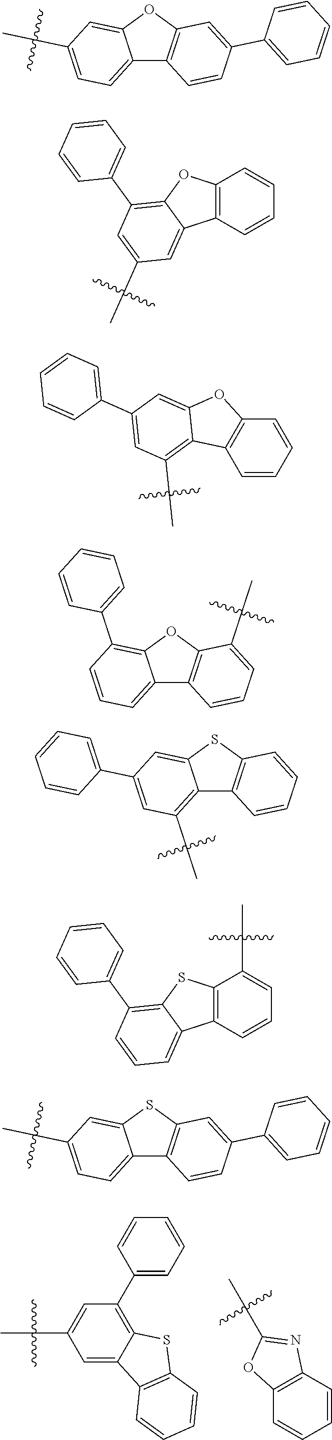

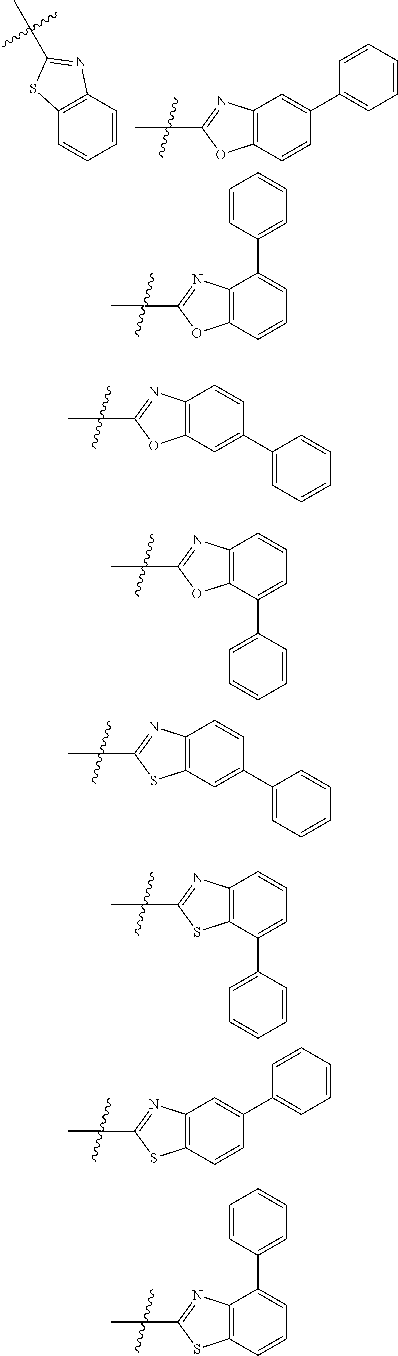

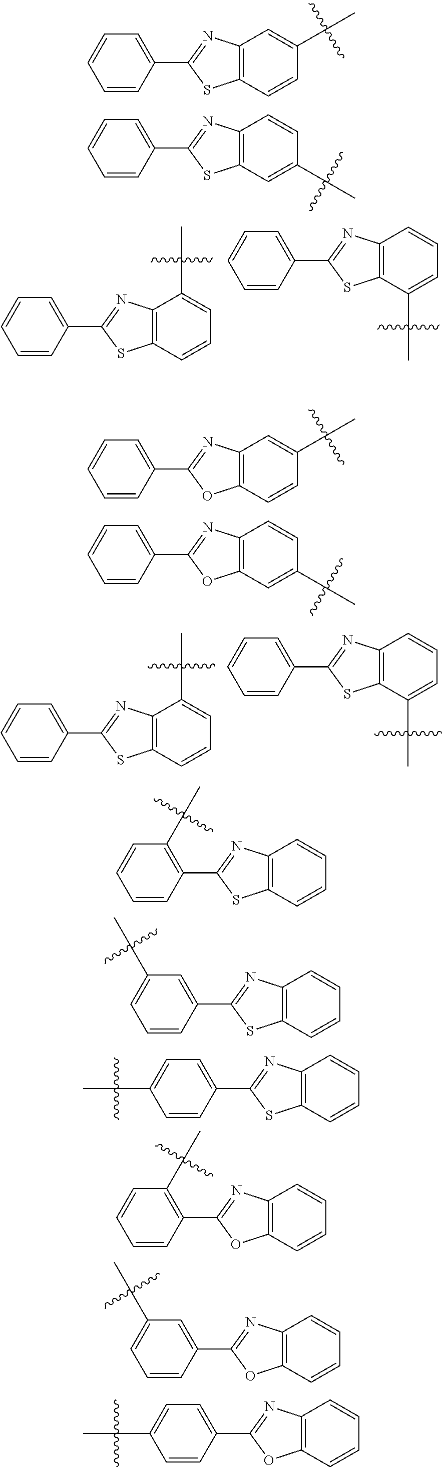

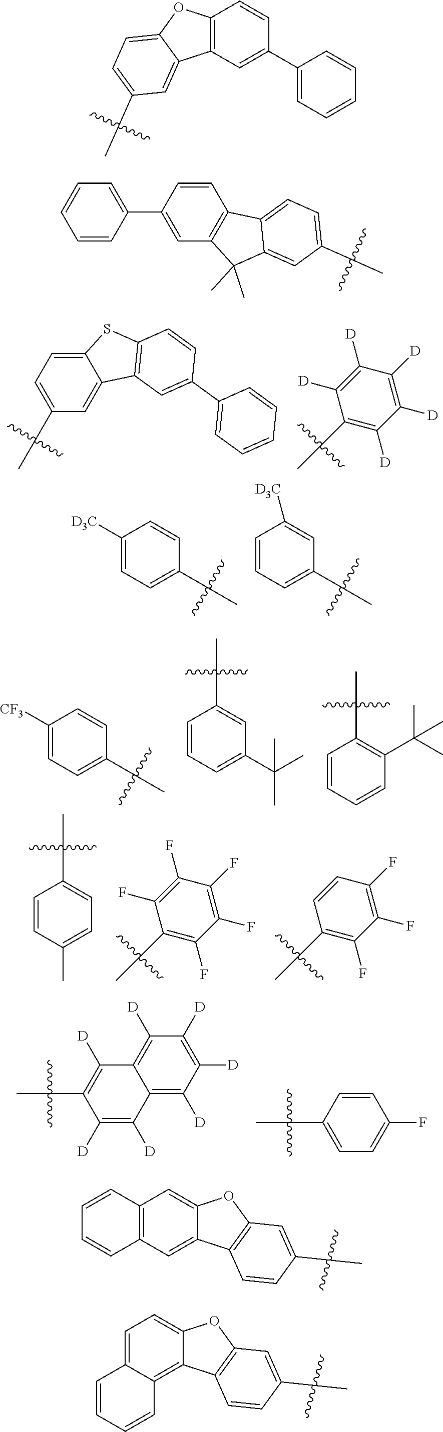

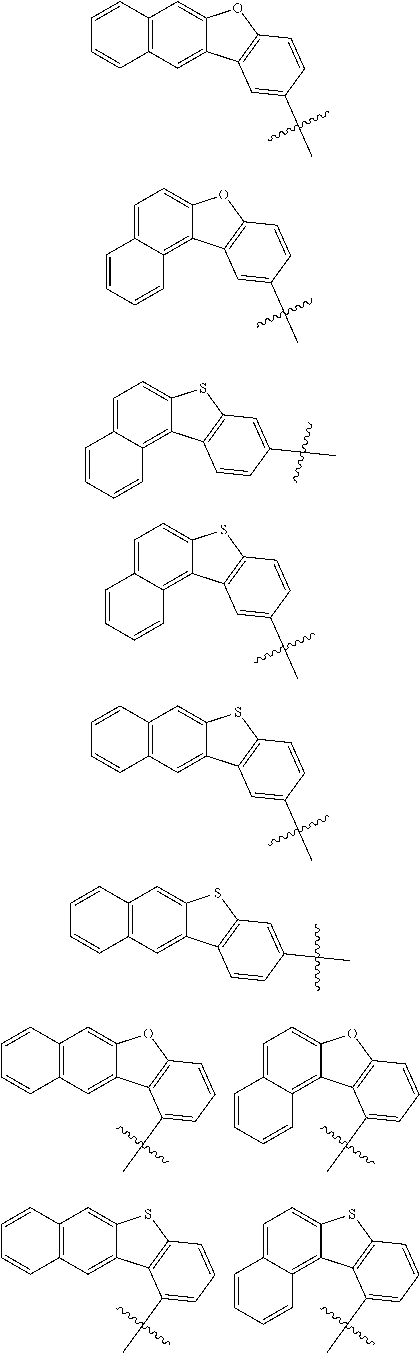

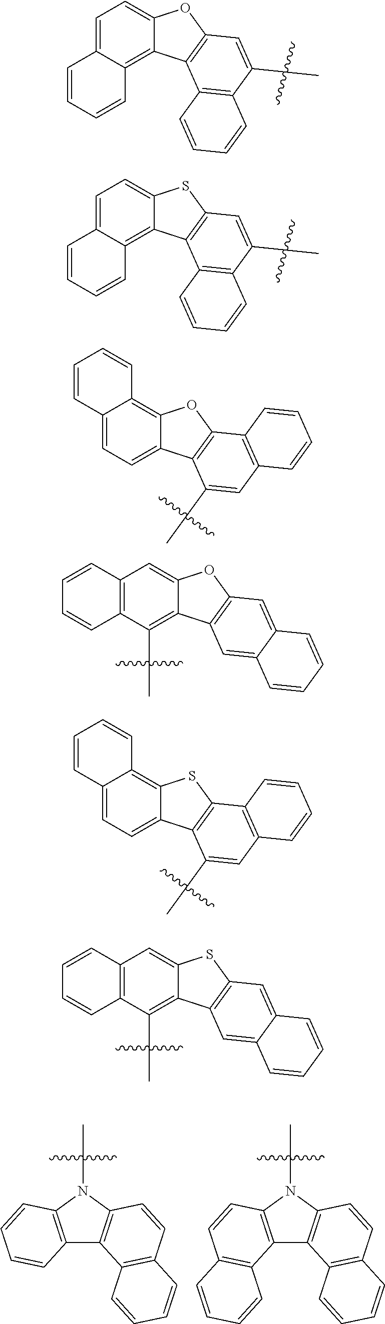

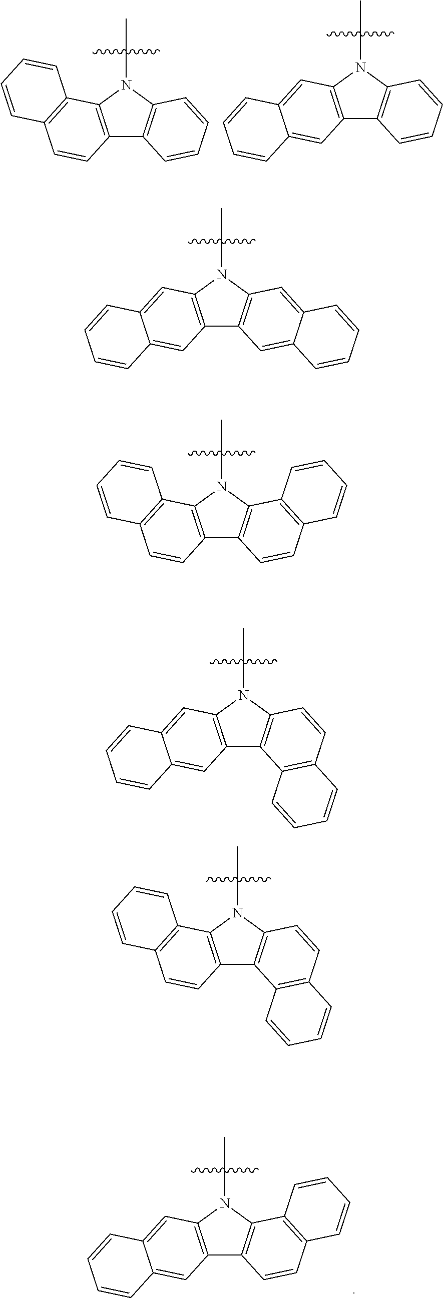

In some embodiments, Ar₃ is selected from a substituted or unsubstituted aryl having 6 to 18 carbon atoms, or a substituted or unsubstituted heteroaryl having 12 to 18 carbon atoms; substituents of Ar₃ are each independently selected from deuterium, a fluorine, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, or a deuterophenyl.

In some embodiments, Ar₃ is selected from the group consisting of the following groups:

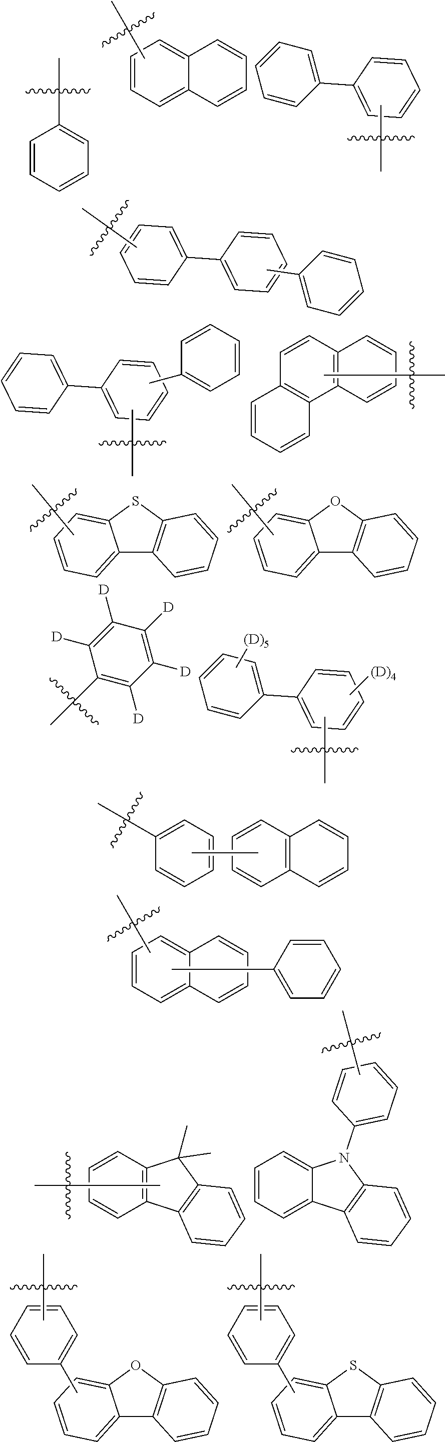

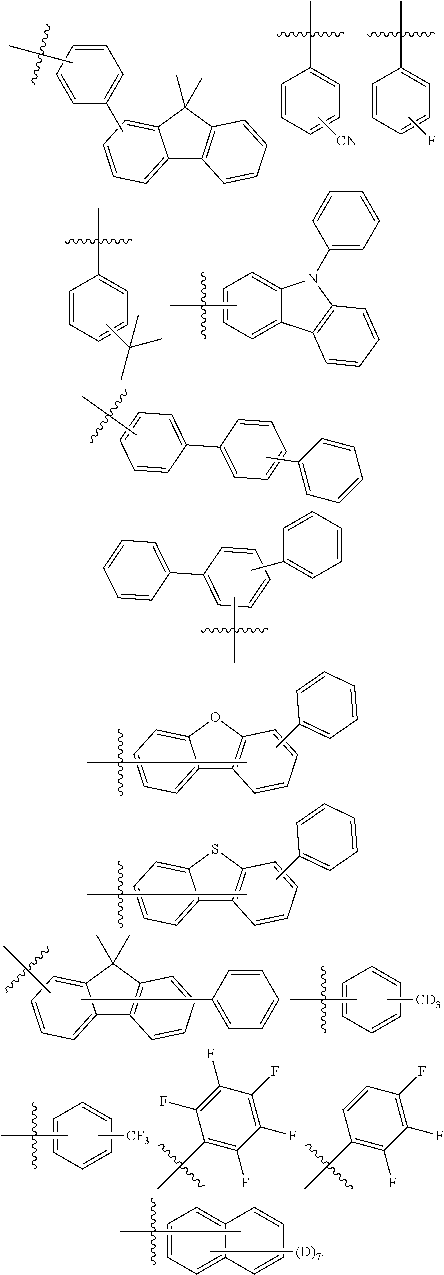

In some embodiments, Ar₃ is selected from the group consisting of the following groups:

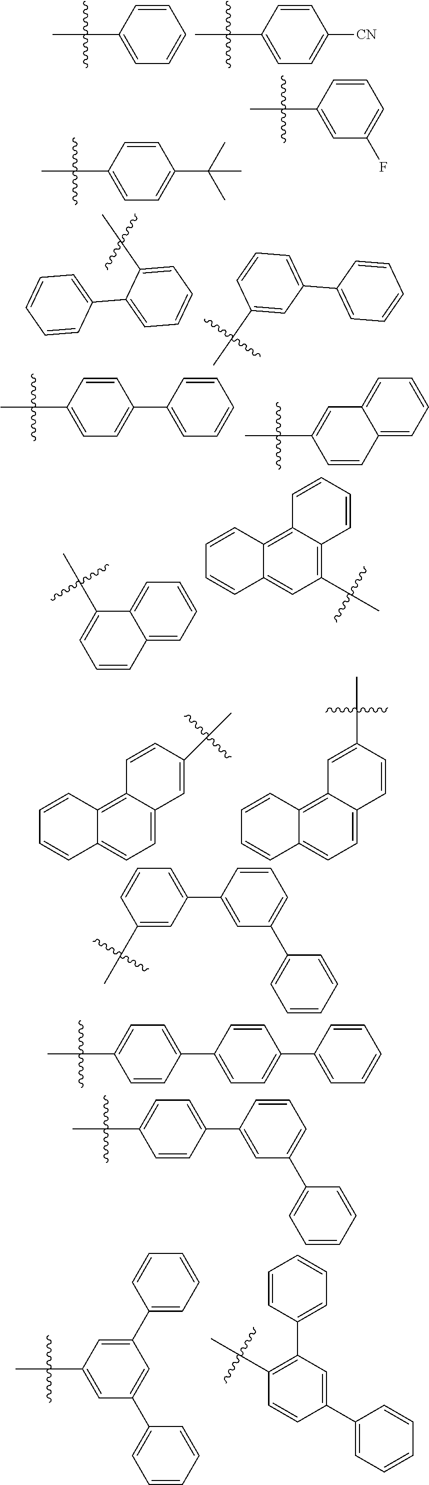

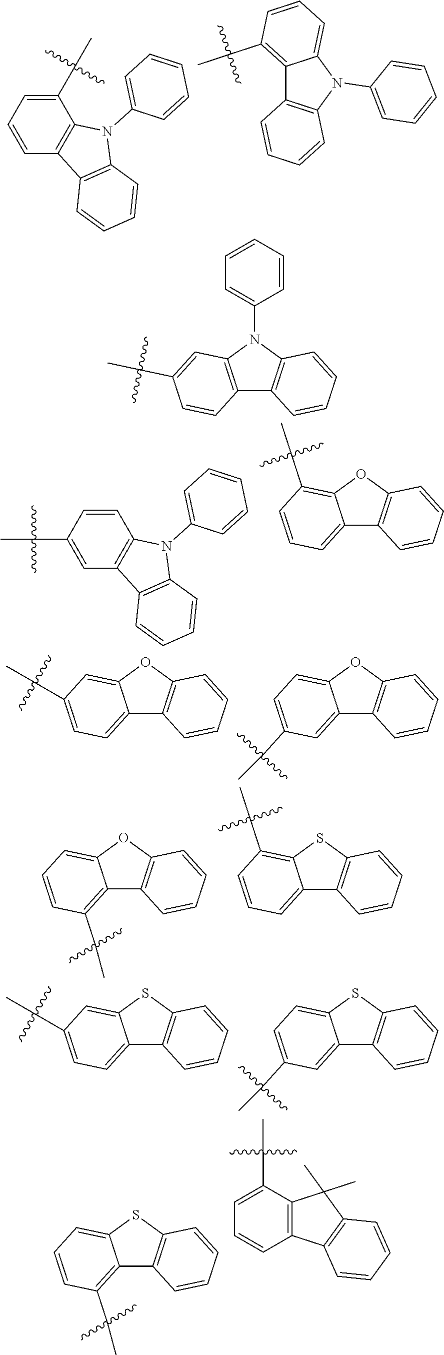

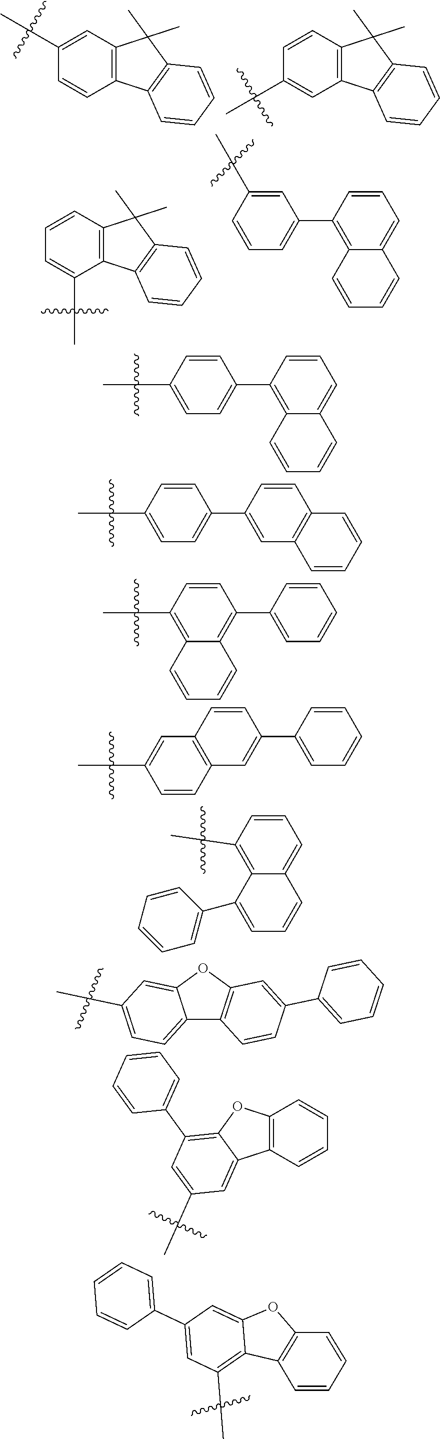

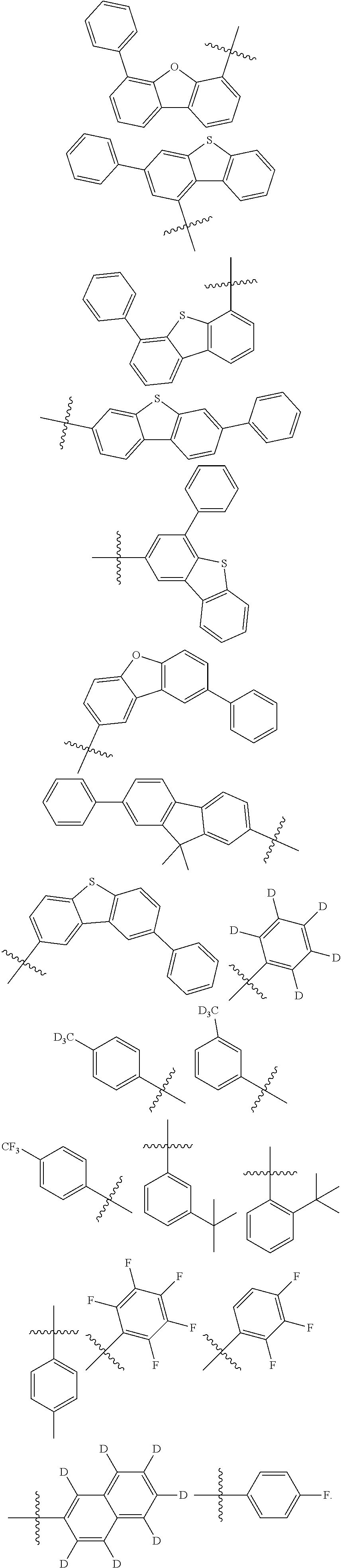

In some embodiments, L, L₁, and L₂ are identical or different, and are each independently selected from a single bond, a substituted or unsubstituted arylene having 6 to 15 carbon atoms, or a substituted or unsubstituted heteroarylene having 5 to 18 carbon atoms.

In some embodiments, L, L₁, and L₂ are identical or different, and are each independently selected from a single bond; a substituted or unsubstituted arylene having 6, 7, 8, 9, 10, 11, 12, 13, 14, or 15 carbon atoms; or a substituted or unsubstituted heteroarylene having 5, 6, 7, 8, 9, 10, 11, 12, 13, 14, 15, 16, 17, or 18 carbon atoms.

Optionally, the substituents of L, L₁, and L₂ are each independently selected from deuterium, a fluorine, a cyano, an alkyl having 1 to 5 carbon atoms, a trialkylsilyl having 3 to 8 carbon atoms, a fluoroalkyl having 1 to 4 carbon atoms, a deuterated alkyl having 1 to 4 carbon atoms, a phenyl, or a naphthyl.

In some embodiments, L is selected from a single bond, a substituted or unsubstituted phenylene, a substituted or unsubstituted naphthylene, a substituted or unsubstituted biphenylene, a substituted or unsubstituted pyridylidene, a substituted or unsubstituted dibenzothiophenylene, or a substituted or unsubstituted dibenzofuranylene.

In some embodiments, L₁ and L₂ are each independently selected from a single bond, a substituted or unsubstituted phenylene, a substituted or unsubstituted naphthylene, a substituted or unsubstituted biphenylene, a substituted or unsubstituted fluorenylidene, a substituted or unsubstituted phenanthrylene, a substituted or unsubstituted dibenzothiophenylene, a substituted or unsubstituted dibenzofuranylene, a substituted or unsubstituted carbazolylene, a substituted or unsubstituted pyridylidene, a substituted or unsubstituted benzoxazolylene, or a substituted or unsubstituted benzothiazolylene.

Optionally, the substituents of L, L₁, and L₂ are identical or different, and each independently selected from deuterium, a fluorine, a cyano, a methyl, an ethyl, an isopropyl, a tert-butyl, a trifluoromethyl, a trideuteromethyl, a trimethylsilyl, or a phenyl.

Optionally, L, L₁, and L₂ are each independently selected from a single bond or a substituted or unsubstituted group Q. The unsubstituted group Q is selected from the following groups:

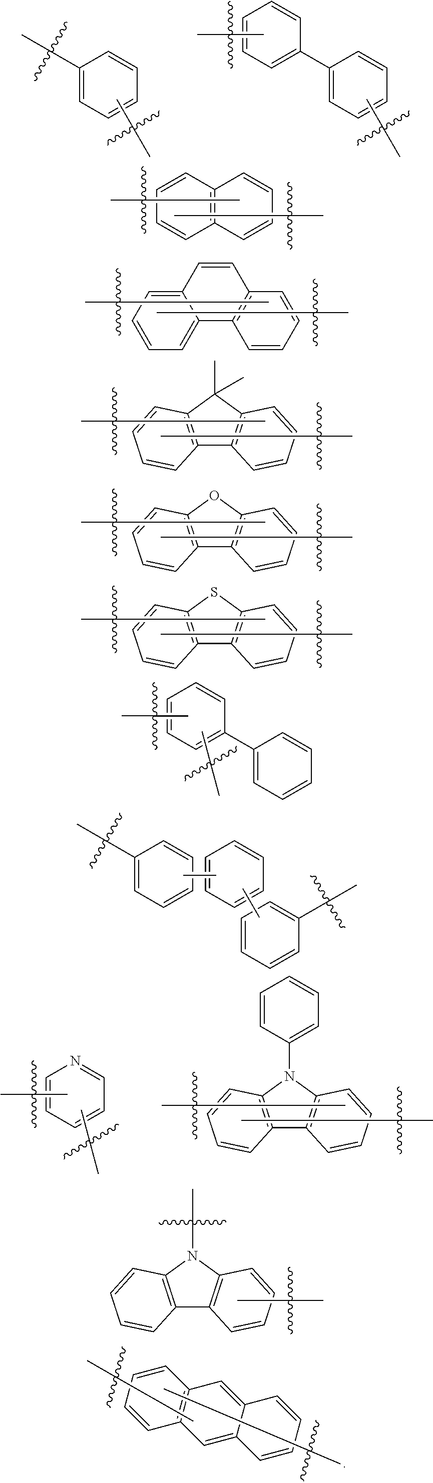

The substituted group Q each have one or more than two substituents, the substituents of the substituted group Q are each independently selected from deuterium, a fluorine, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, a dibenzofuranyl, a dibenzothiophenyl, or a carbazolyl; and when the number of the substituents of the group Q is greater than 1, the substituents are identical or different.

In some embodiments, L, L₁, and L₂ are each independently selected from a single bond or the group consisting of the following groups:

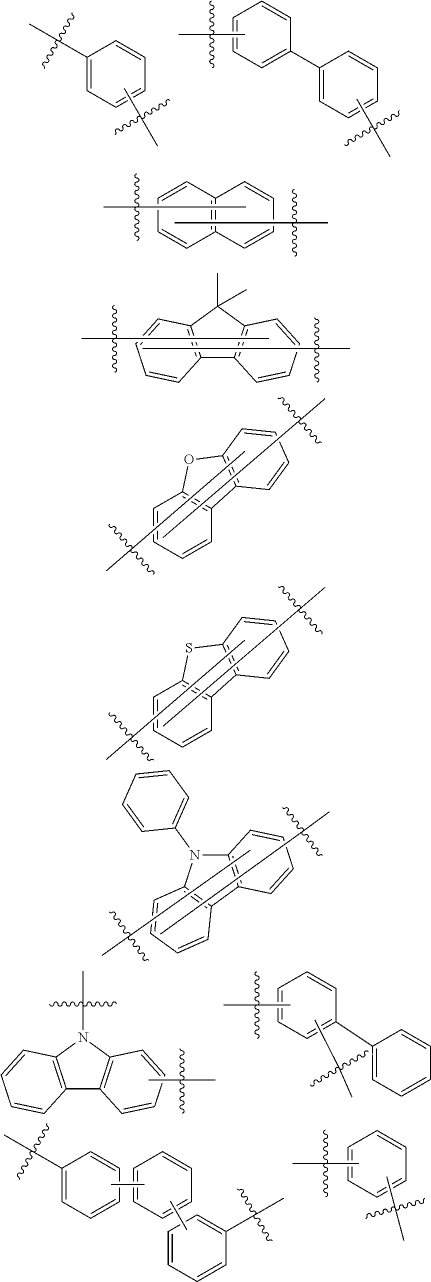

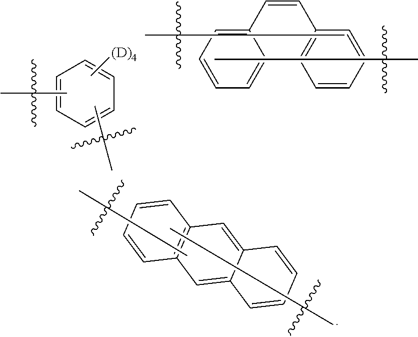

In some embodiments, L is selected from a single bond or the group consisting of the following groups:

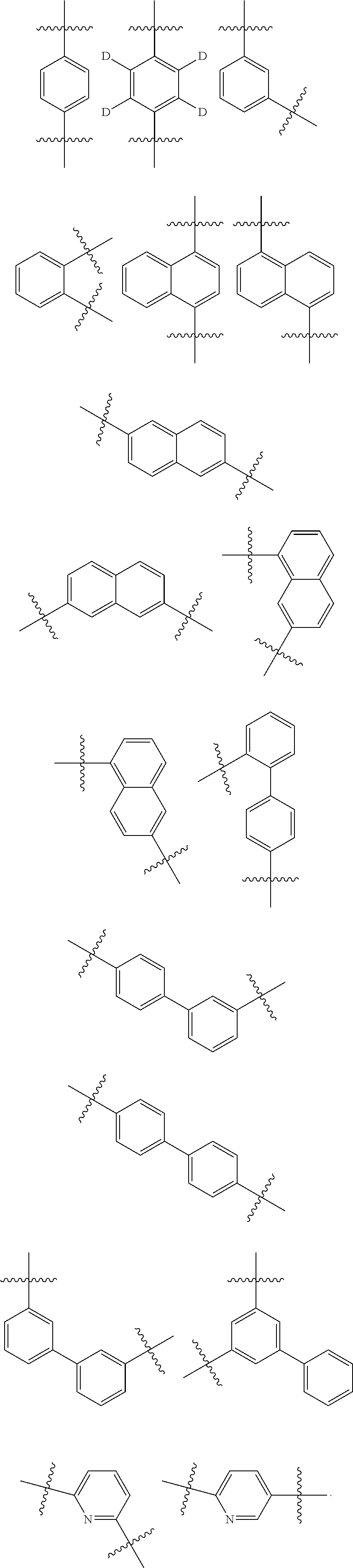

In some embodiments, L₁ and L₂ are each independently selected from a single bond or the group consisting of the following groups:

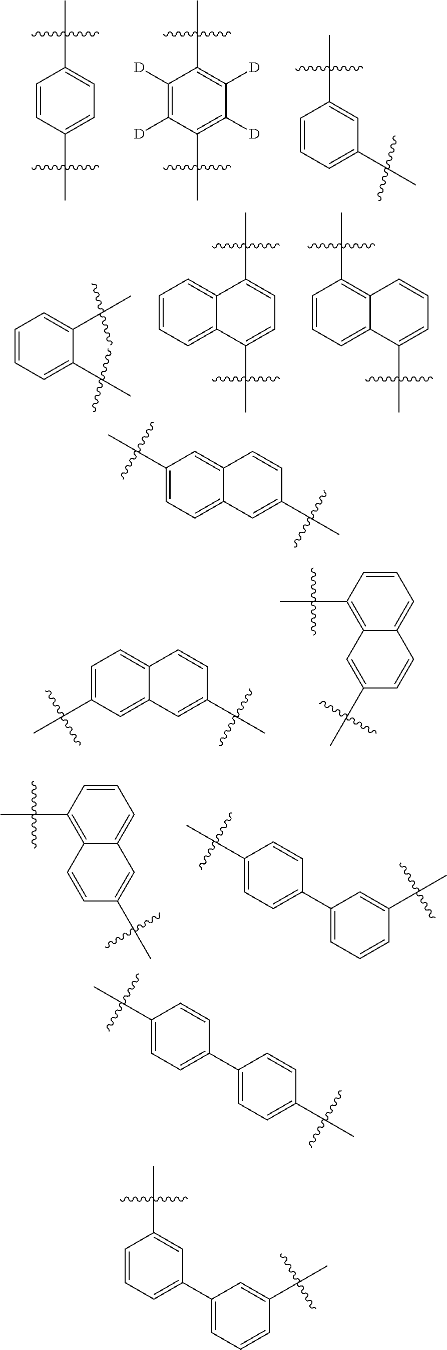

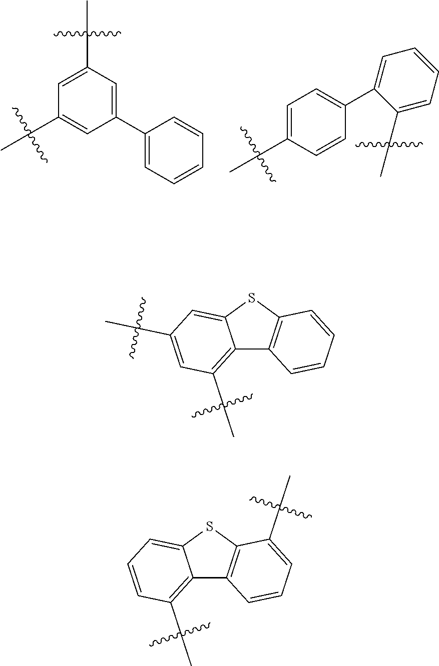 [SMILES: *c1cc(*)cc(-c2ccccc2)c1 ; *c1ccc(-c2ccccc2*)cc1 ; *c1cc(*)c2c(c1)sc1ccccc12 ; *c1cccc2c1sc1cccc(*)c12]

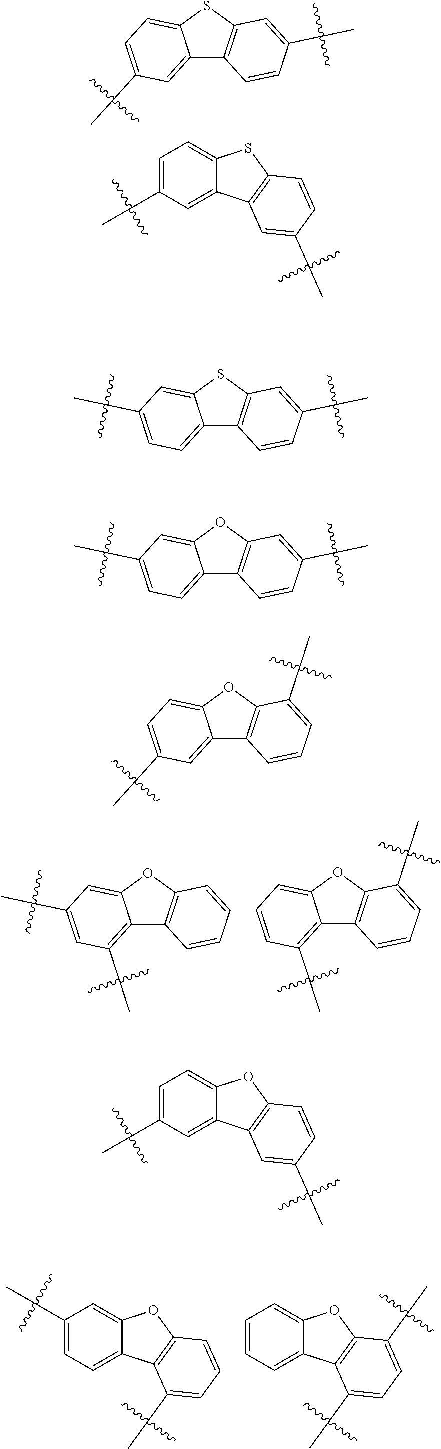

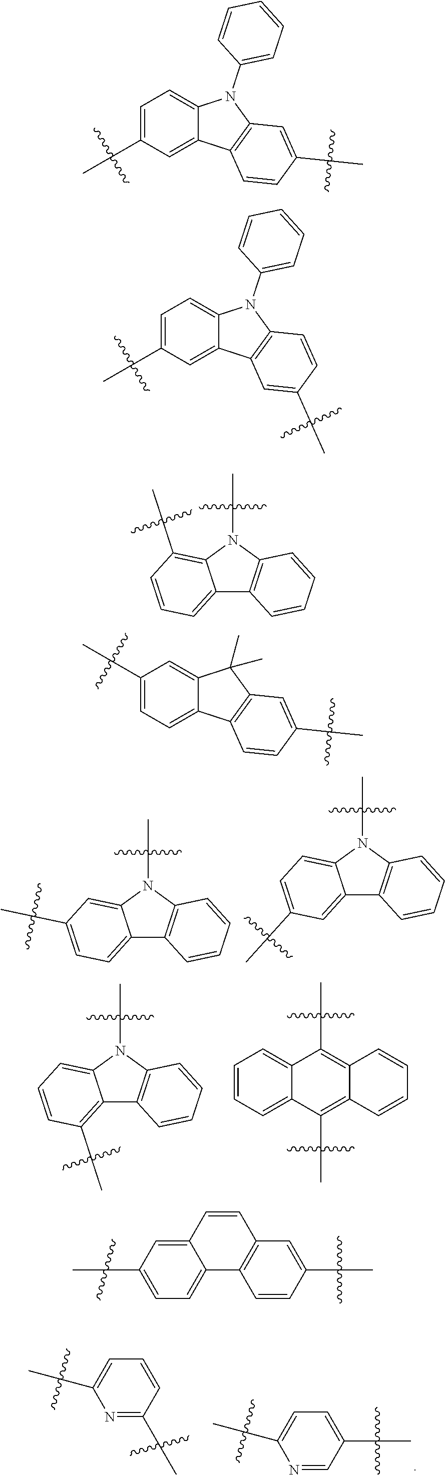

In some embodiments,

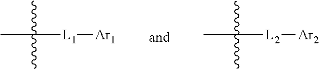

are each independently selected from the group consisting of the following groups:

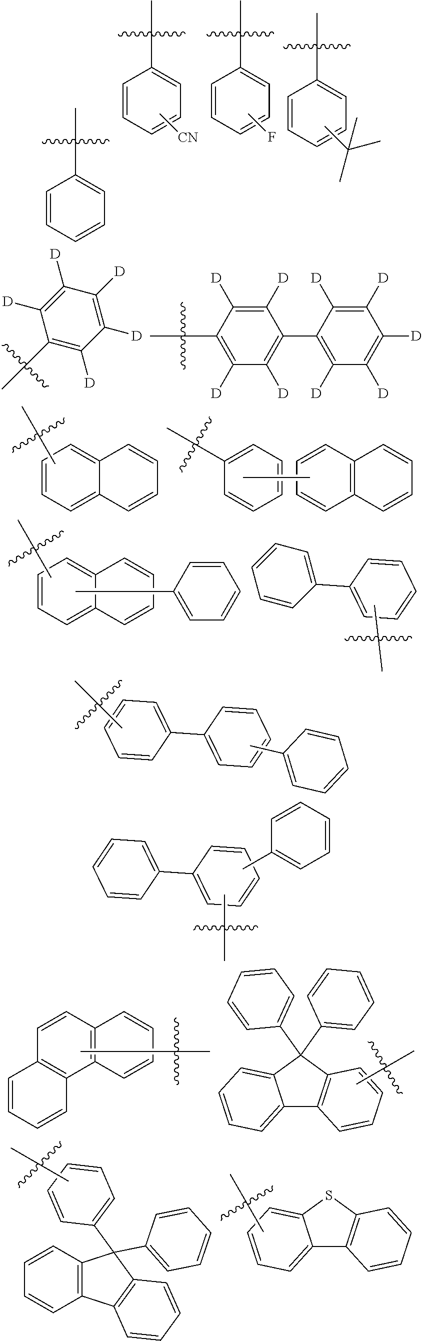

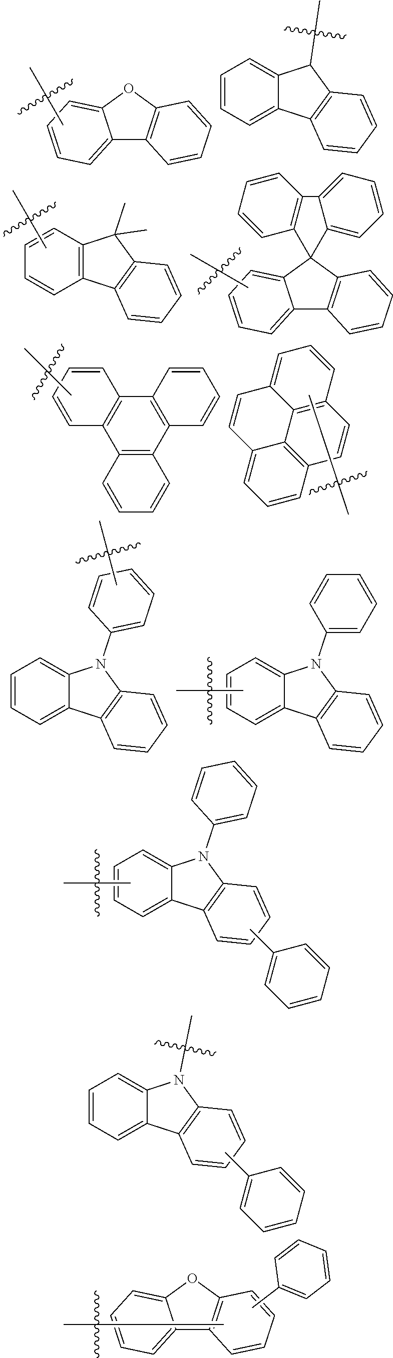

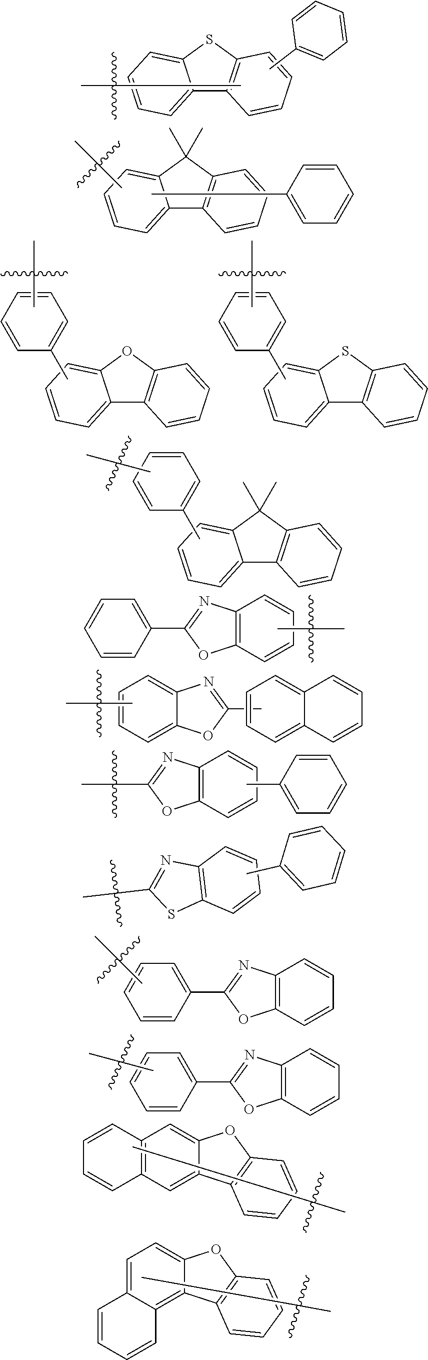

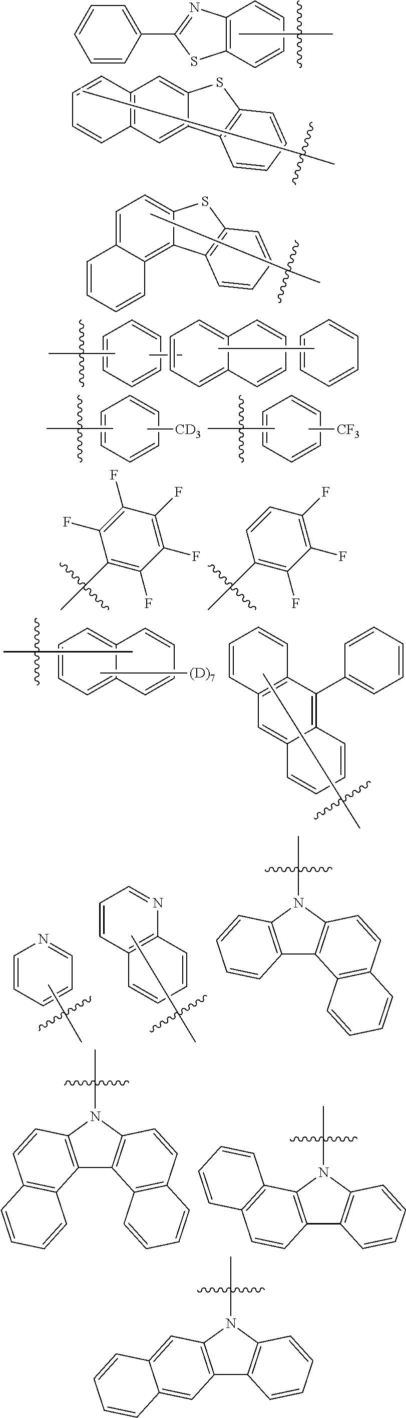

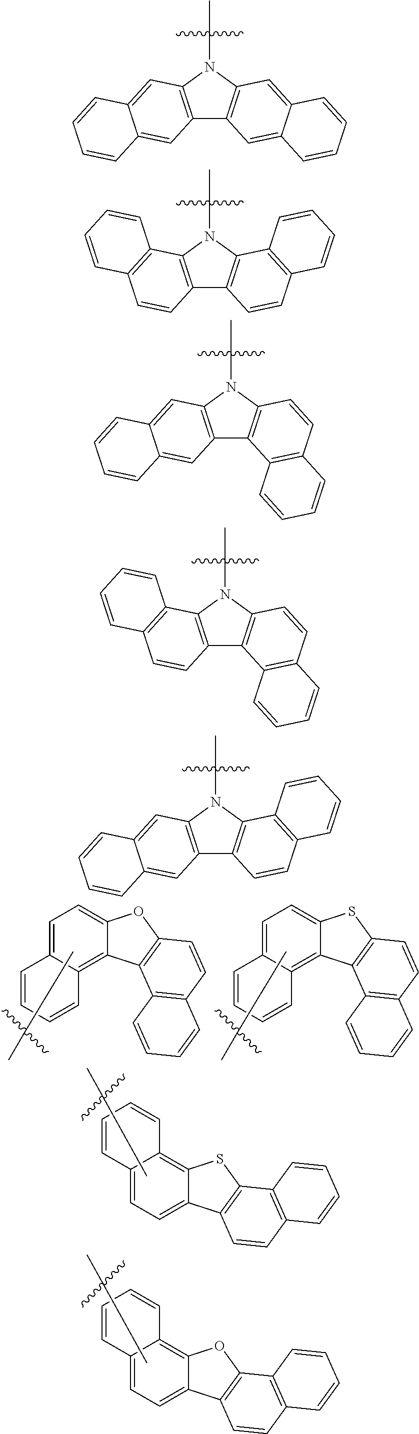

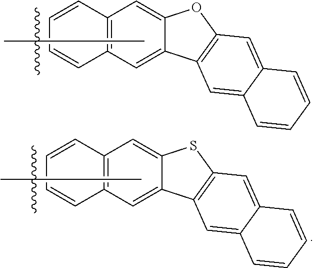

Optionally, each R is identical or different, and is each independently selected from hydrogen, deuterium, a cyano, a fluorine, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, a dibenzofuranyl, a dibenzothiophenyl, or a carbazolyl.

Optionally, each R₁ is hydrogen, deuterium, or cyano.

Optionally, the nitrogen-containing compound is selected from the group consisting of the compounds shown below:

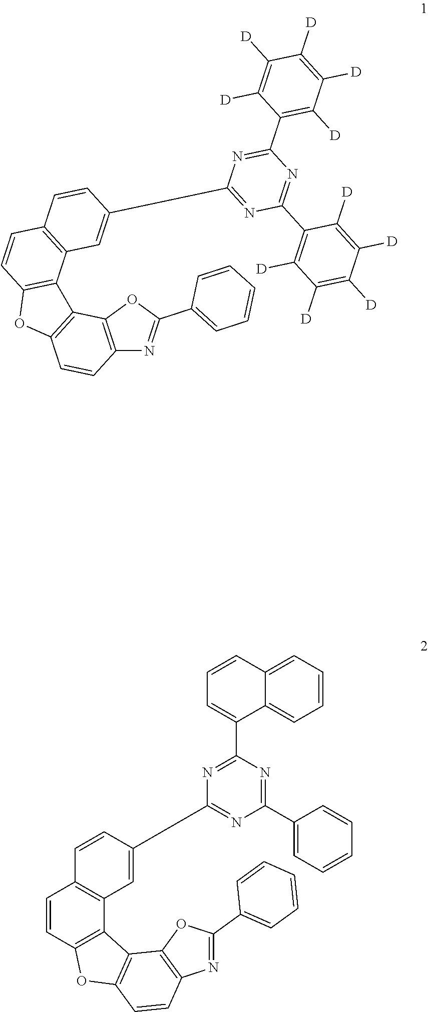 [SMILES: [2H]c1c([2H])c([2H])c(-c2nc(-c3ccc4ccc5oc6ccc7nc(-c8ccccc8)oc7c6c5c4c3)nc(-c3c([2H])c([2H])c([2H])c([2H])c3[2H])n2)c([2H])c1[2H] ; c1ccc(-c2nc(-c3ccc4ccc5oc6ccc7nc(-c8ccccc8)oc7c6c5c4c3)nc(-c3cccc4ccccc34)n2)cc1]

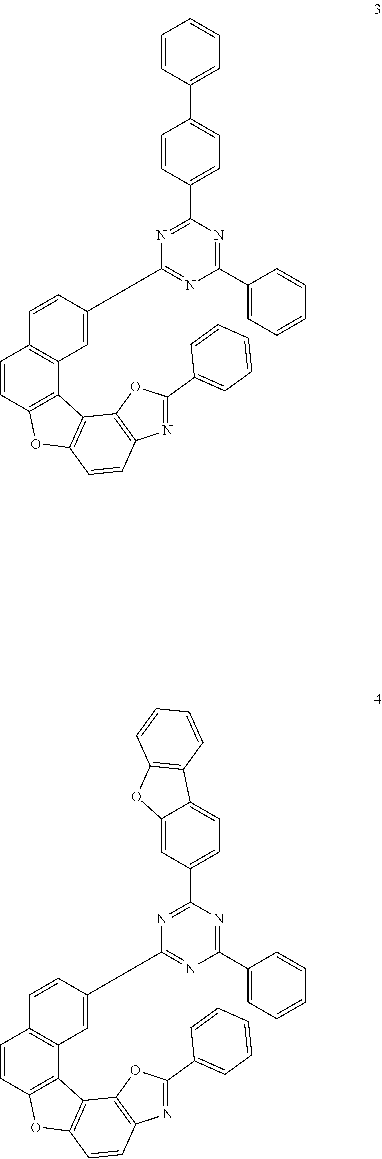 [SMILES: c1ccc(-c2ccc(-c3nc(-c4ccccc4)nc(-c4ccc5ccc6oc7ccc8nc(-c9ccccc9)oc8c7c6c5c4)n3)cc2)cc1 ; c1ccc(-c2nc(-c3ccc4c(c3)oc3ccccc34)nc(-c3ccc4ccc5oc6ccc7nc(-c8ccccc8)oc7c6c5c4c3)n2)cc1]

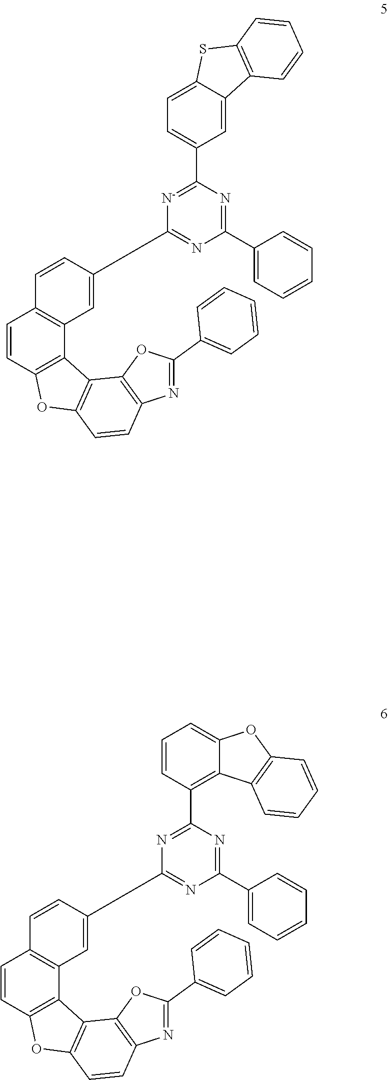

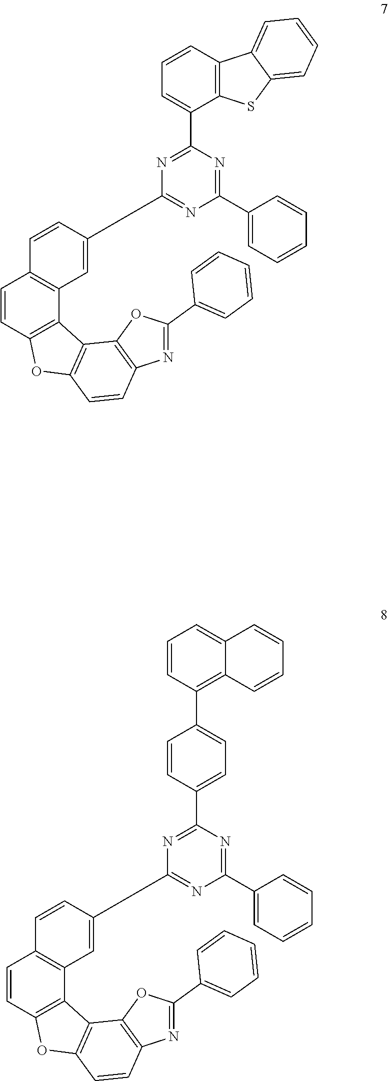 [SMILES: c1ccc(-c2nc(-c3ccc4ccc5oc6ccc7nc(-c8ccccc8)oc7c6c5c4c3)nc(-c3cccc4c3sc3ccccc34)n2)cc1 ; c1ccc(-c2nc(-c3ccc(-c4cccc5ccccc45)cc3)nc(-c3ccc4ccc5oc6ccc7nc(-c8ccccc8)oc7c6c5c4c3)n2)cc1]

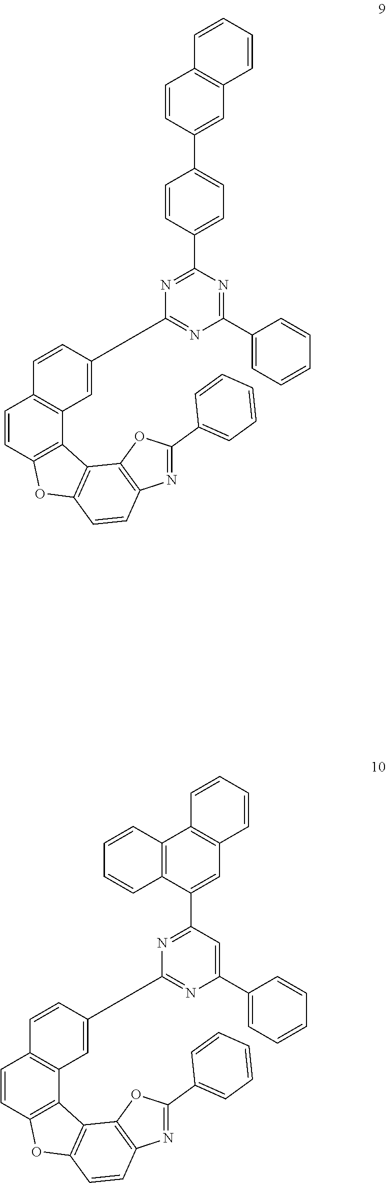 [SMILES: c1ccc(-c2nc(-c3ccc(-c4ccc5ccccc5c4)cc3)nc(-c3ccc4ccc5oc6ccc7nc(-c8ccccc8)oc7c6c5c4c3)n2)cc1 ; c1ccc(-c2cc(-c3cc4ccccc4c4ccccc34)nc(-c3ccc4ccc5oc6ccc7nc(-c8ccccc8)oc7c6c5c4c3)n2)cc1]

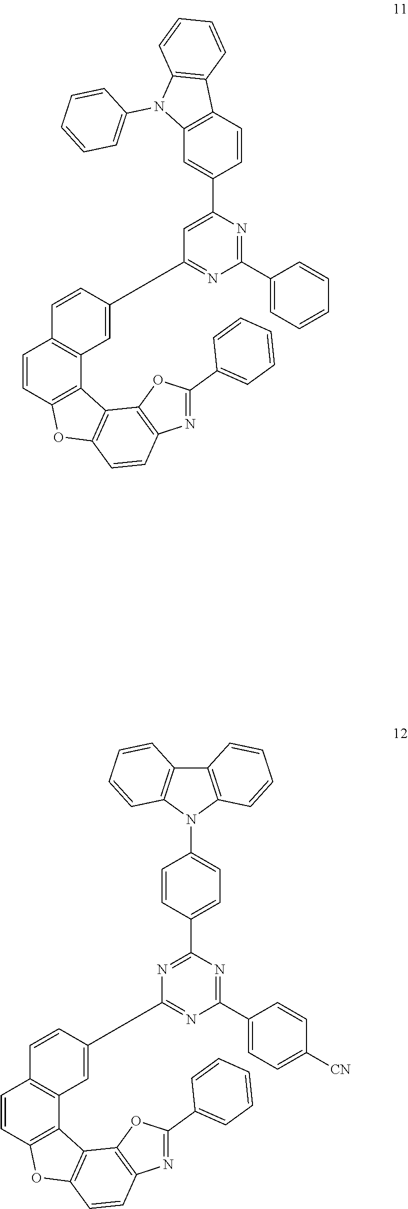 [SMILES: c1ccc(-c2nc(-c3ccc4ccc5oc6ccc7nc(-c8ccccc8)oc7c6c5c4c3)cc(-c3ccc4c5ccccc5n(-c5ccccc5)c4c3)n2)cc1 ; N#Cc1ccc(-c2nc(-c3ccc(-n4c5ccccc5c5ccccc54)cc3)nc(-c3ccc4ccc5oc6ccc7nc(-c8ccccc8)oc7c6c5c4c3)n2)cc1]

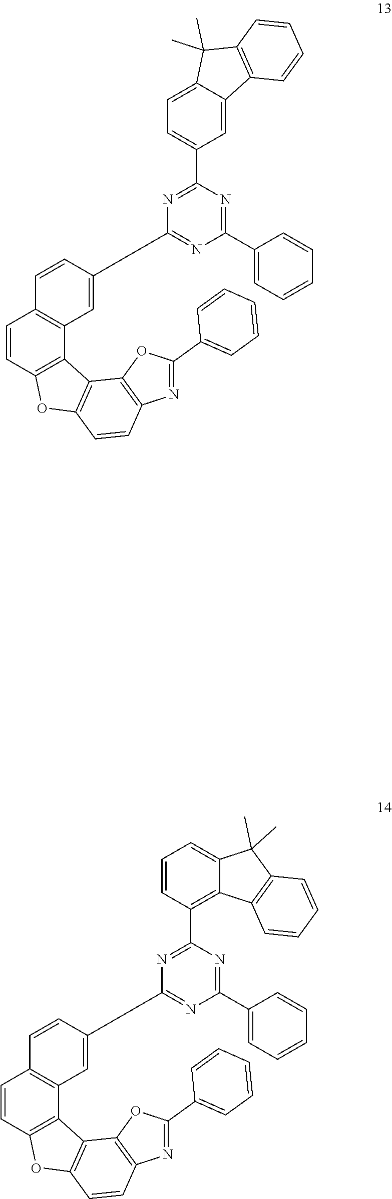 [SMILES: CC1(C)c2ccccc2-c2cc(-c3nc(-c4ccccc4)nc(-c4ccc5ccc6oc7ccc8nc(-c9ccccc9)oc8c7c6c5c4)n3)ccc21 ; CC1(C)c2ccccc2-c2c(-c3nc(-c4ccccc4)nc(-c4ccc5ccc6oc7ccc8nc(-c9ccccc9)oc8c7c6c5c4)n3)cccc21]

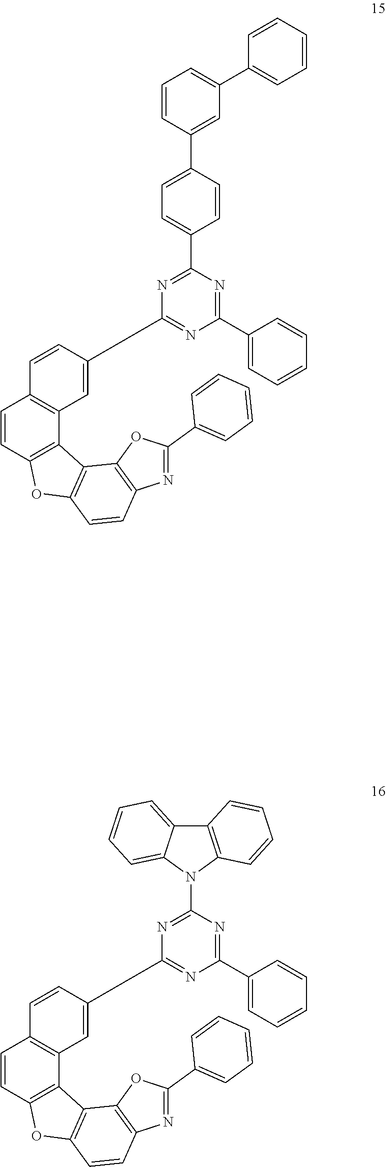 [SMILES: c1ccc(-c2cccc(-c3ccc(-c4nc(-c5ccccc5)nc(-c5ccc6ccc7oc8ccc9nc(-c%10ccccc%10)oc9c8c7c6c5)n4)cc3)c2)cc1 ; c1ccc(-c2nc(-c3ccc4ccc5oc6ccc7nc(-c8ccccc8)oc7c6c5c4c3)nc(-n3c4ccccc4c4ccccc43)n2)cc1]

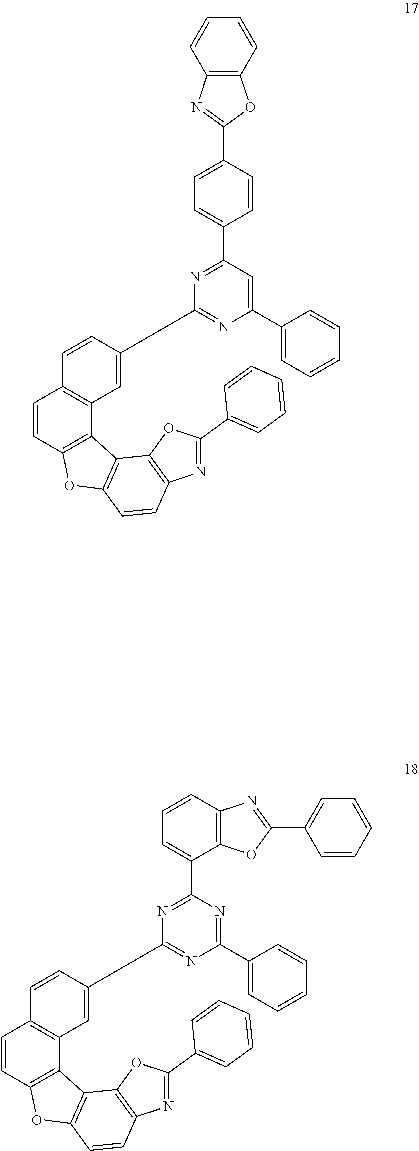 [SMILES: c1ccc(-c2cc(-c3ccc(-c4nc5ccccc5o4)cc3)nc(-c3ccc4ccc5oc6ccc7nc(-c8ccccc8)oc7c6c5c4c3)n2)cc1 ; c1ccc(-c2nc(-c3ccc4ccc5oc6ccc7nc(-c8ccccc8)oc7c6c5c4c3)nc(-c3cccc4nc(-c5ccccc5)oc34)n2)cc1]

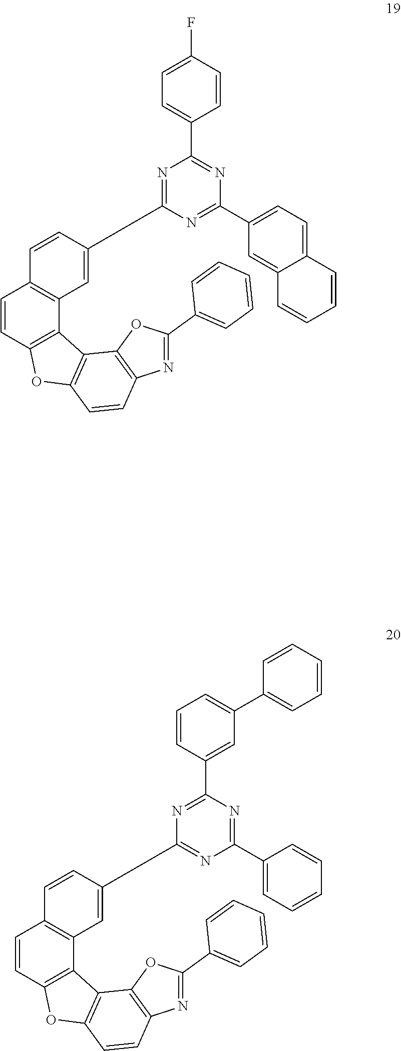 [SMILES: Fc1ccc(-c2nc(-c3ccc4ccccc4c3)nc(-c3ccc4ccc5oc6ccc7nc(-c8ccccc8)oc7c6c5c4c3)n2)cc1 ; c1ccc(-c2cccc(-c3nc(-c4ccccc4)nc(-c4ccc5ccc6oc7ccc8nc(-c9ccccc9)oc8c7c6c5c4)n3)c2)cc1]

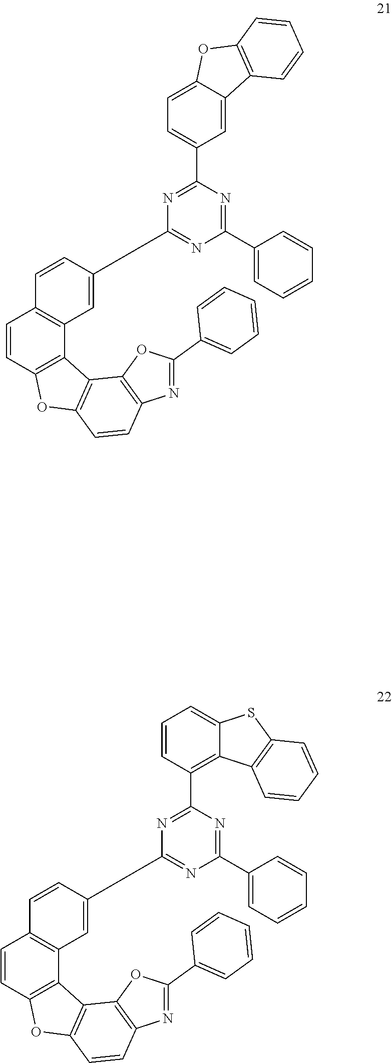 [SMILES: c1ccc(-c2nc(-c3ccc4oc5ccccc5c4c3)nc(-c3ccc4ccc5oc6ccc7nc(-c8ccccc8)oc7c6c5c4c3)n2)cc1 ; c1ccc(-c2nc(-c3ccc4ccc5oc6ccc7nc(-c8ccccc8)oc7c6c5c4c3)nc(-c3cccc4sc5ccccc5c34)n2)cc1]

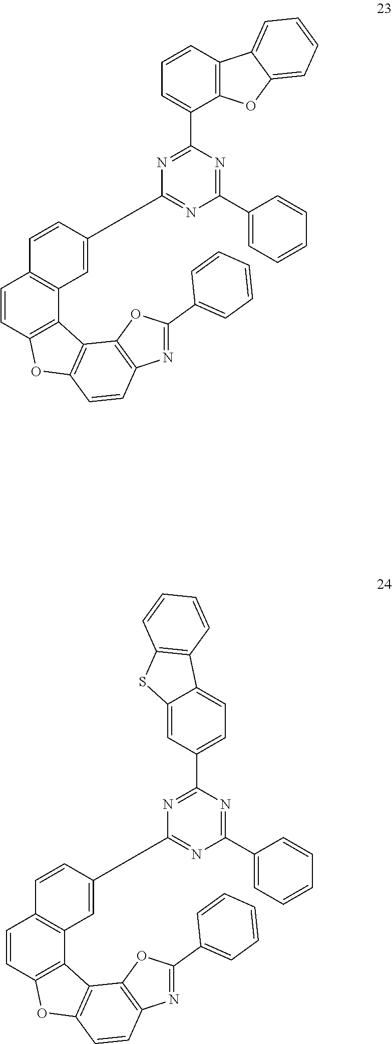 [SMILES: c1ccc(-c2nc(-c3ccc4ccc5oc6ccc7nc(-c8ccccc8)oc7c6c5c4c3)nc(-c3cccc4c3oc3ccccc34)n2)cc1 ; c1ccc(-c2nc(-c3ccc4c(c3)sc3ccccc34)nc(-c3ccc4ccc5oc6ccc7nc(-c8ccccc8)oc7c6c5c4c3)n2)cc1]

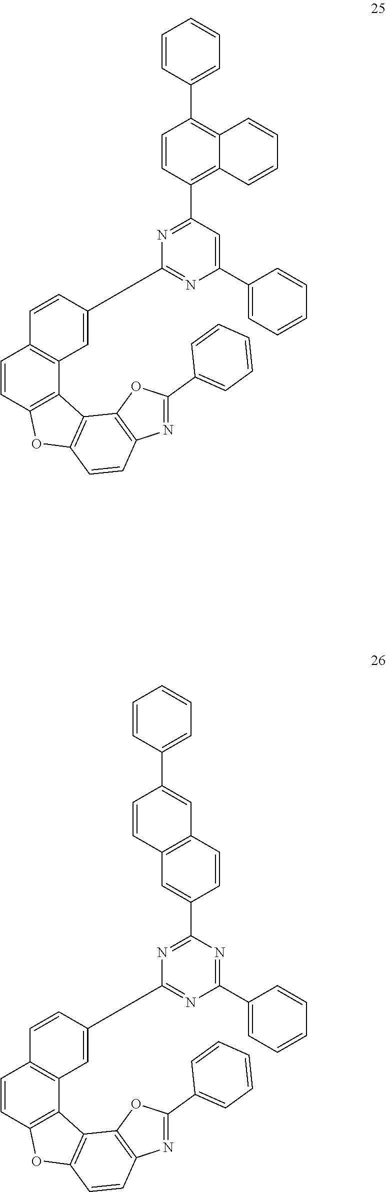 [SMILES: c1ccc(-c2cc(-c3ccc(-c4ccccc4)c4ccccc34)nc(-c3ccc4ccc5oc6ccc7nc(-c8ccccc8)oc7c6c5c4c3)n2)cc1 ; c1ccc(-c2ccc3cc(-c4nc(-c5ccccc5)nc(-c5ccc6ccc7oc8ccc9nc(-c%10ccccc%10)oc9c8c7c6c5)n4)ccc3c2)cc1]

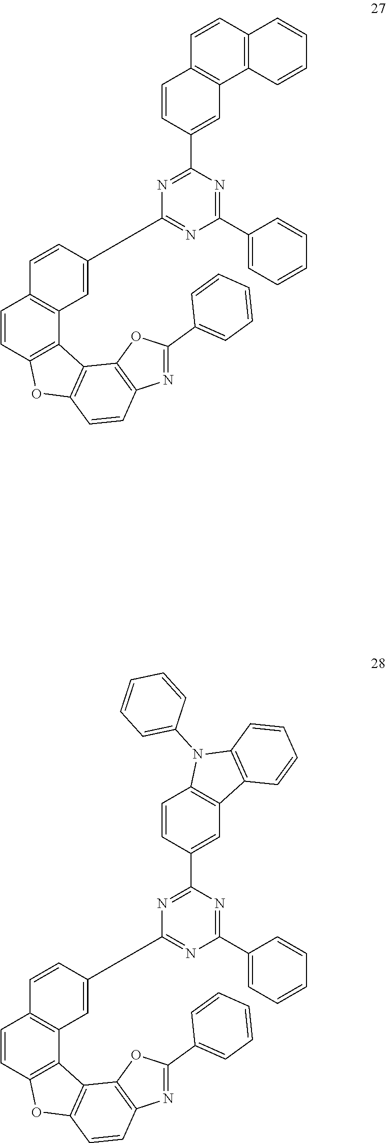 [SMILES: c1ccc(-c2nc(-c3ccc4ccc5ccccc5c4c3)nc(-c3ccc4ccc5oc6ccc7nc(-c8ccccc8)oc7c6c5c4c3)n2)cc1 ; c1ccc(-c2nc(-c3ccc4ccc5oc6ccc7nc(-c8ccccc8)oc7c6c5c4c3)nc(-c3ccc4c(c3)c3ccccc3n4-c3ccccc3)n2)cc1]

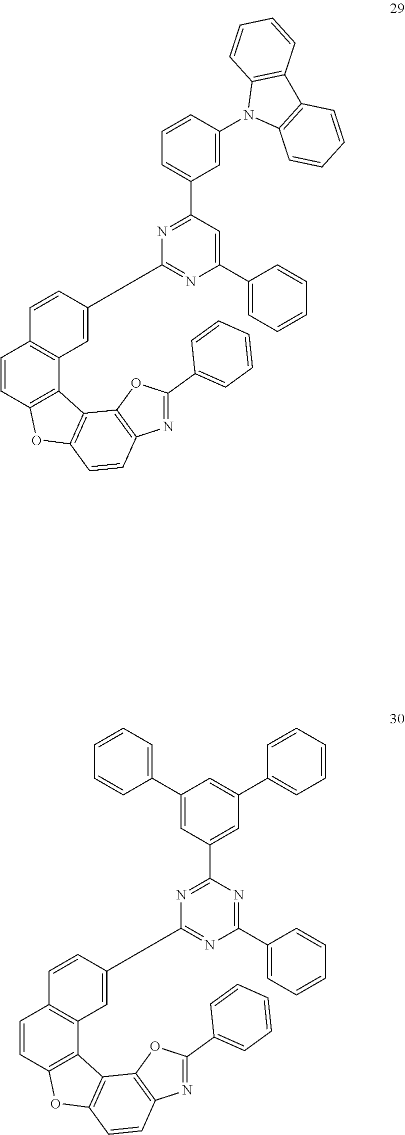 [SMILES: c1ccc(-c2cc(-c3cccc(-n4c5ccccc5c5ccccc54)c3)nc(-c3ccc4ccc5oc6ccc7nc(-c8ccccc8)oc7c6c5c4c3)n2)cc1 ; c1ccc(-c2cc(-c3ccccc3)cc(-c3nc(-c4ccccc4)nc(-c4ccc5ccc6oc7ccc8nc(-c9ccccc9)oc8c7c6c5c4)n3)c2)cc1]

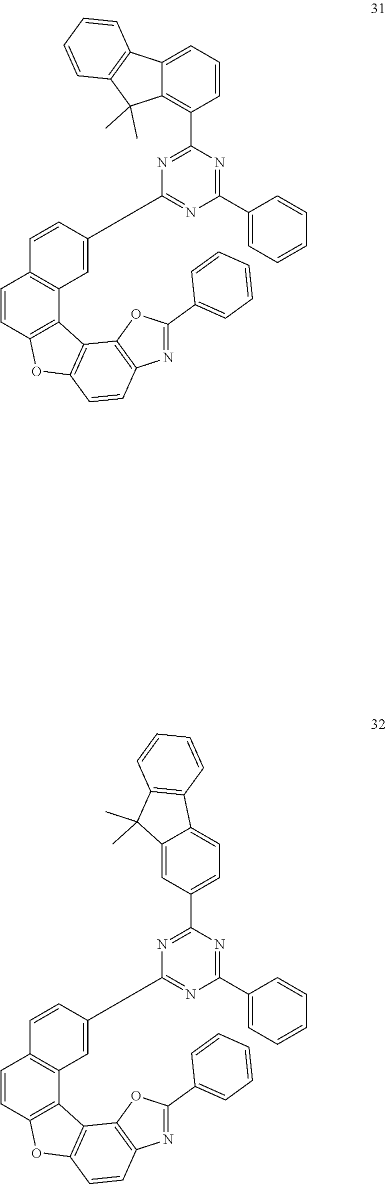 [SMILES: CC1(C)c2ccccc2-c2cccc(-c3nc(-c4ccccc4)nc(-c4ccc5ccc6oc7ccc8nc(-c9ccccc9)oc8c7c6c5c4)n3)c21 ; CC1(C)c2ccccc2-c2ccc(-c3nc(-c4ccccc4)nc(-c4ccc5ccc6oc7ccc8nc(-c9ccccc9)oc8c7c6c5c4)n3)cc21]

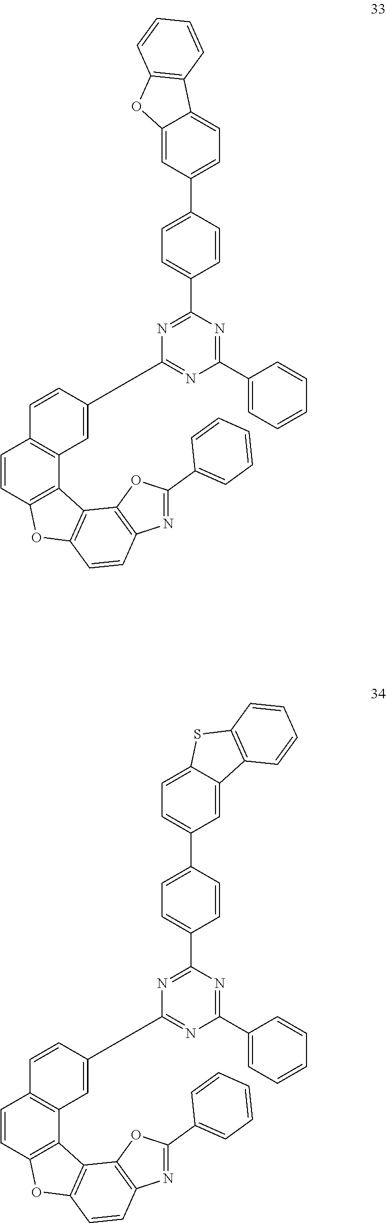 [SMILES: c1ccc(-c2nc(-c3ccc(-c4ccc5c(c4)oc4ccccc45)cc3)nc(-c3ccc4ccc5oc6ccc7nc(-c8ccccc8)oc7c6c5c4c3)n2)cc1 ; c1ccc(-c2nc(-c3ccc(-c4ccc5sc6ccccc6c5c4)cc3)nc(-c3ccc4ccc5oc6ccc7nc(-c8ccccc8)oc7c6c5c4c3)n2)cc1]

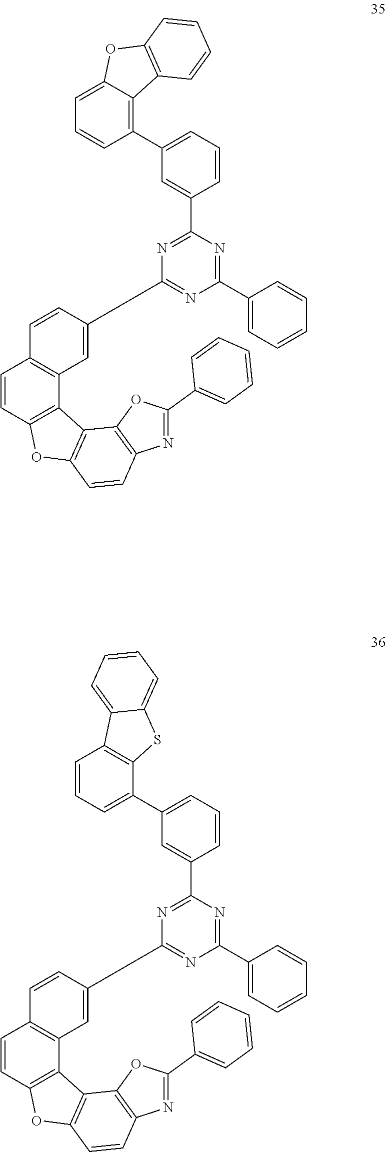 [SMILES: c1ccc(-c2nc(-c3cccc(-c4cccc5oc6ccccc6c45)c3)nc(-c3ccc4ccc5oc6ccc7nc(-c8ccccc8)oc7c6c5c4c3)n2)cc1 ; c1ccc(-c2nc(-c3cccc(-c4cccc5c4sc4ccccc45)c3)nc(-c3ccc4ccc5oc6ccc7nc(-c8ccccc8)oc7c6c5c4c3)n2)cc1]

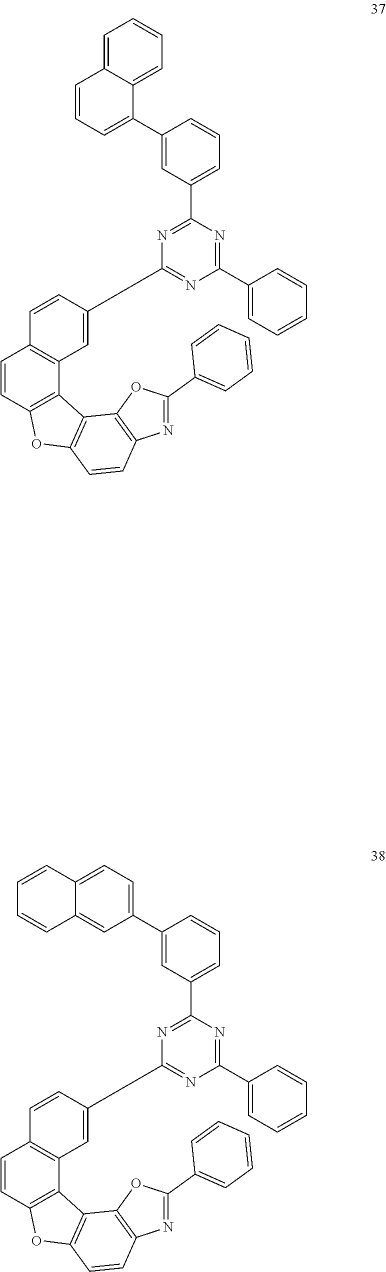 [SMILES: c1ccc(-c2nc(-c3cccc(-c4cccc5ccccc45)c3)nc(-c3ccc4ccc5oc6ccc7nc(-c8ccccc8)oc7c6c5c4c3)n2)cc1 ; c1ccc(-c2nc(-c3cccc(-c4ccc5ccccc5c4)c3)nc(-c3ccc4ccc5oc6ccc7nc(-c8ccccc8)oc7c6c5c4c3)n2)cc1]

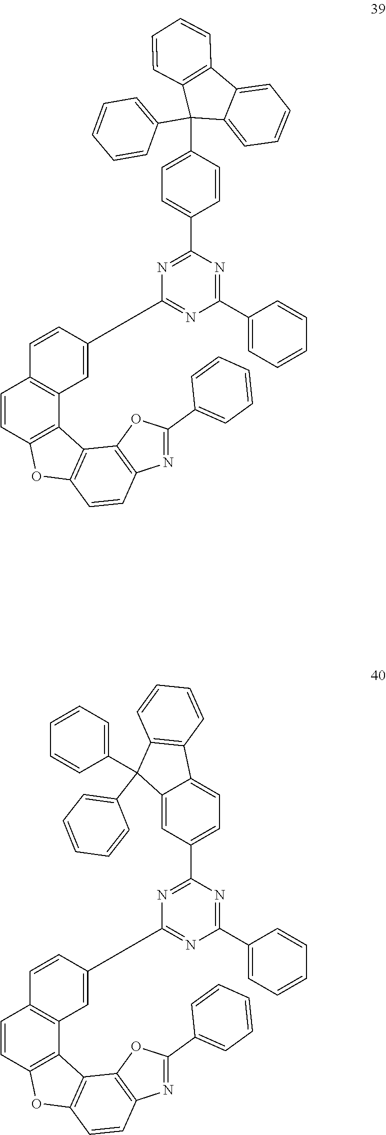 [SMILES: c1ccc(-c2nc(-c3ccc(C4(c5ccccc5)c5ccccc5-c5ccccc54)cc3)nc(-c3ccc4ccc5oc6ccc7nc(-c8ccccc8)oc7c6c5c4c3)n2)cc1 ; c1ccc(-c2nc(-c3ccc4c(c3)C(c3ccccc3)(c3ccccc3)c3ccccc3-4)nc(-c3ccc4ccc5oc6ccc7nc(-c8ccccc8)oc7c6c5c4c3)n2)cc1]

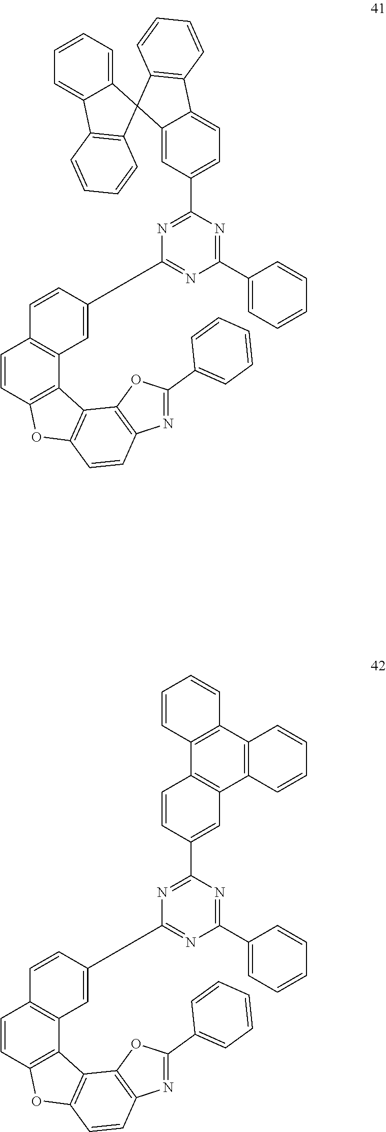 [SMILES: c1ccc(-c2nc(-c3ccc4c(c3)C3(c5ccccc5-c5ccccc53)c3ccccc3-4)nc(-c3ccc4ccc5oc6ccc7nc(-c8ccccc8)oc7c6c5c4c3)n2)cc1 ; c1ccc(-c2nc(-c3ccc4c5ccccc5c5ccccc5c4c3)nc(-c3ccc4ccc5oc6ccc7nc(-c8ccccc8)oc7c6c5c4c3)n2)cc1]

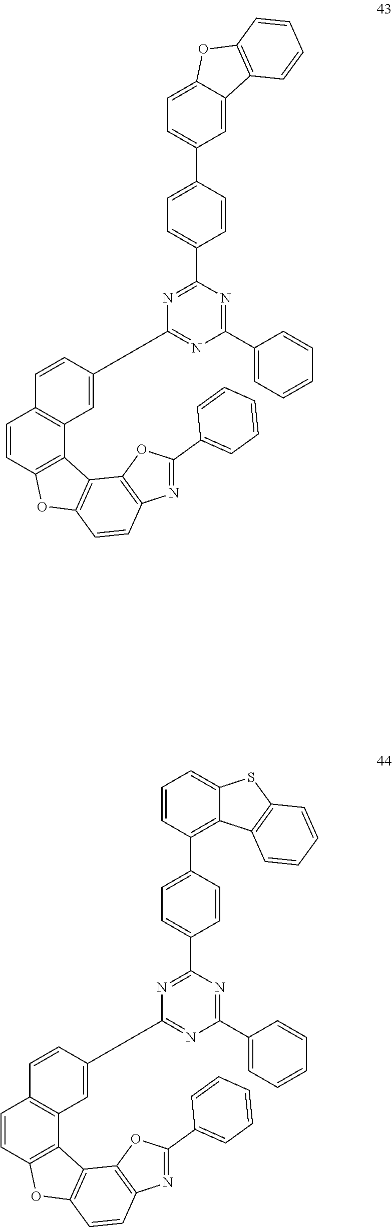 [SMILES: c1ccc(-c2nc(-c3ccc(-c4ccc5oc6ccccc6c5c4)cc3)nc(-c3ccc4ccc5oc6ccc7nc(-c8ccccc8)oc7c6c5c4c3)n2)cc1 ; c1ccc(-c2nc(-c3ccc(-c4cccc5sc6ccccc6c45)cc3)nc(-c3ccc4ccc5oc6ccc7nc(-c8ccccc8)oc7c6c5c4c3)n2)cc1]

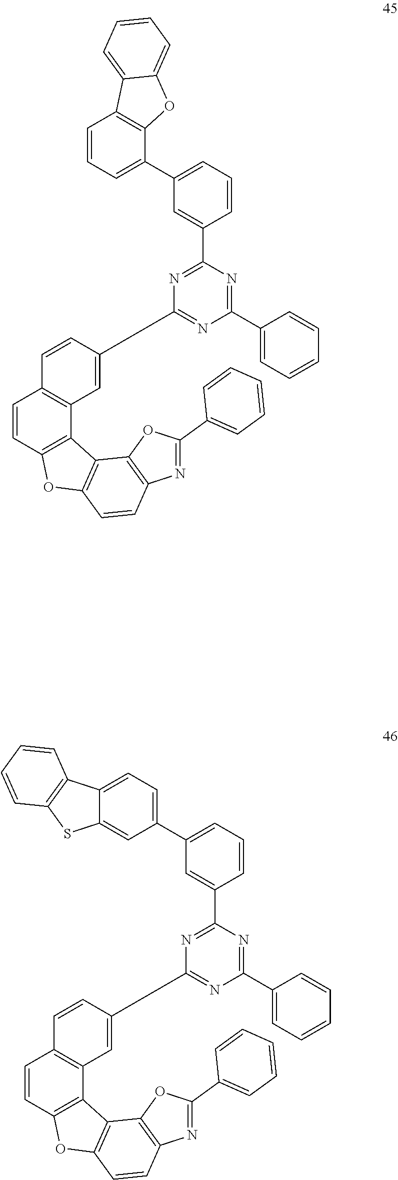 [SMILES: c1ccc(-c2nc(-c3cccc(-c4cccc5c4oc4ccccc45)c3)nc(-c3ccc4ccc5oc6ccc7nc(-c8ccccc8)oc7c6c5c4c3)n2)cc1 ; c1ccc(-c2nc(-c3cccc(-c4ccc5c(c4)sc4ccccc45)c3)nc(-c3ccc4ccc5oc6ccc7nc(-c8ccccc8)oc7c6c5c4c3)n2)cc1]

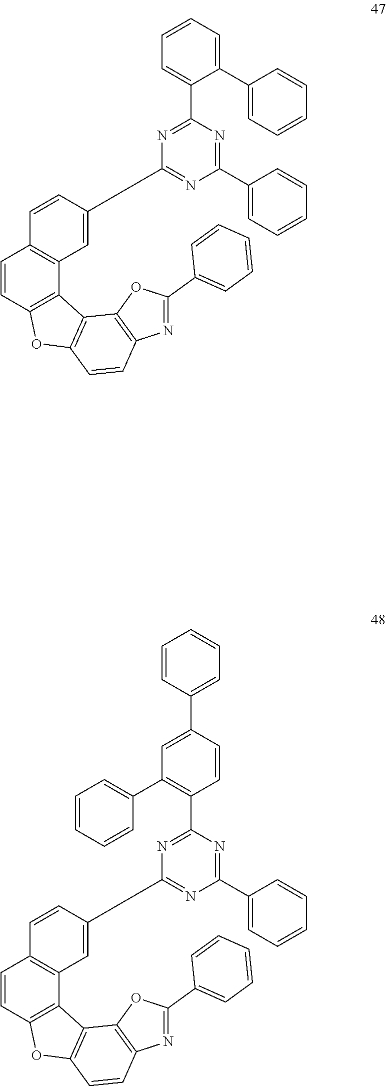 [SMILES: c1ccc(-c2nc(-c3ccc4ccc5oc6ccc7nc(-c8ccccc8)oc7c6c5c4c3)nc(-c3ccccc3-c3ccccc3)n2)cc1 ; c1ccc(-c2ccc(-c3nc(-c4ccccc4)nc(-c4ccc5ccc6oc7ccc8nc(-c9ccccc9)oc8c7c6c5c4)n3)c(-c3ccccc3)c2)cc1]

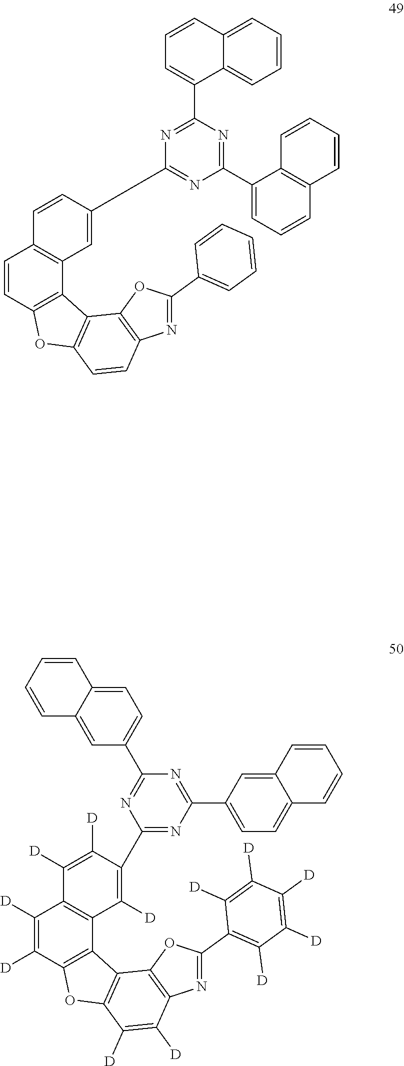 [SMILES: c1ccc(-c2nc3ccc4oc5ccc6ccc(-c7nc(-c8cccc9ccccc89)nc(-c8cccc9ccccc89)n7)cc6c5c4c3o2)cc1 ; [2H]c1c([2H])c([2H])c(-c2nc3c([2H])c([2H])c4oc5c([2H])c([2H])c6c([2H])c([2H])c(-c7nc(-c8ccc9ccccc9c8)nc(-c8ccc9ccccc9c8)n7)c([2H])c6c5c4c3o2)c([2H])c1[2H]]

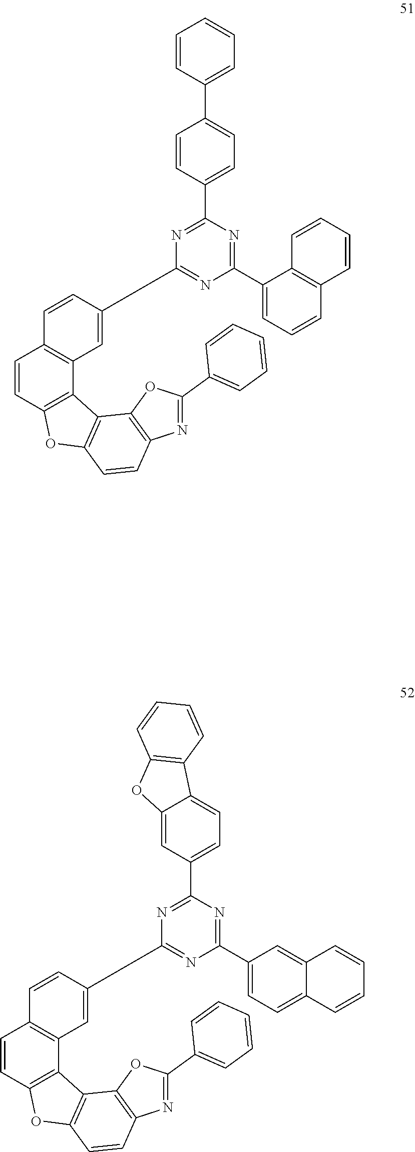 [SMILES: c1ccc(-c2ccc(-c3nc(-c4ccc5ccc6oc7ccc8nc(-c9ccccc9)oc8c7c6c5c4)nc(-c4cccc5ccccc45)n3)cc2)cc1 ; c1ccc(-c2nc3ccc4oc5ccc6ccc(-c7nc(-c8ccc9ccccc9c8)nc(-c8ccc9c(c8)oc8ccccc89)n7)cc6c5c4c3o2)cc1]

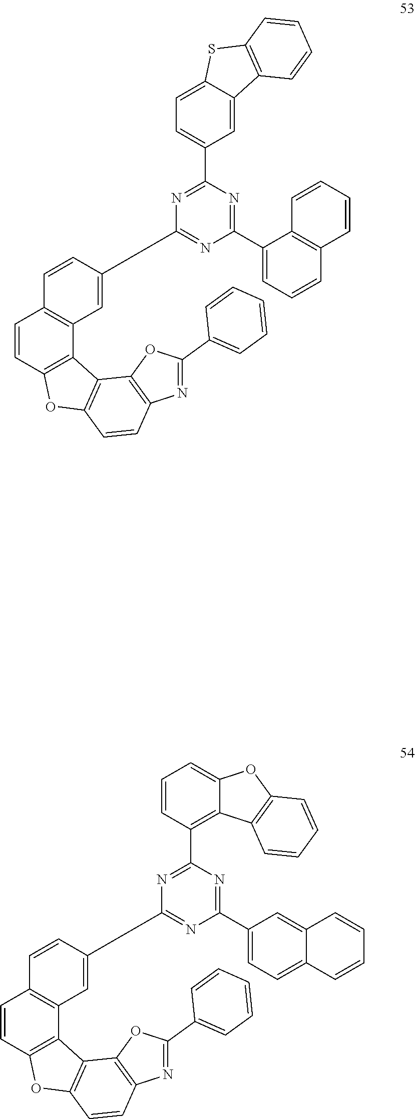 [SMILES: c1ccc(-c2nc3ccc4oc5ccc6ccc(-c7nc(-c8ccc9sc%10ccccc%10c9c8)nc(-c8cccc9ccccc89)n7)cc6c5c4c3o2)cc1 ; c1ccc(-c2nc3ccc4oc5ccc6ccc(-c7nc(-c8ccc9ccccc9c8)nc(-c8cccc9oc%10ccccc%10c89)n7)cc6c5c4c3o2)cc1]

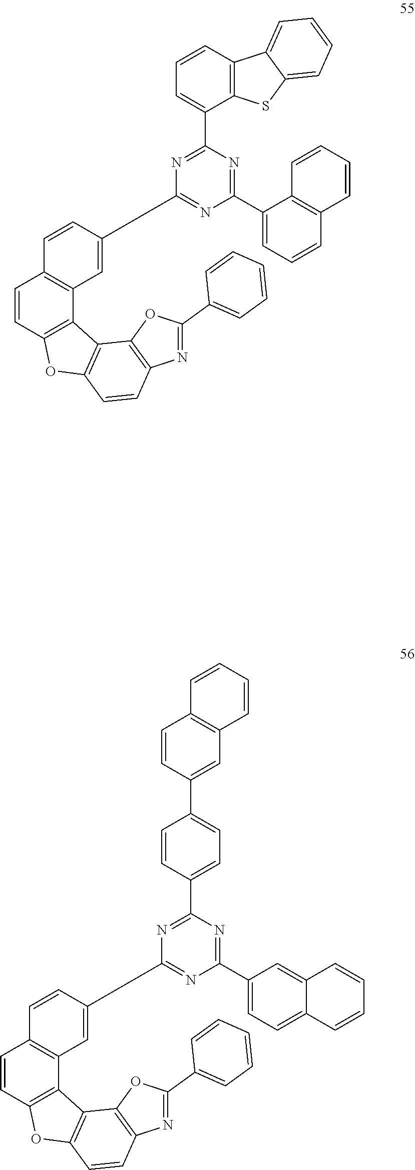 [SMILES: c1ccc(-c2nc3ccc4oc5ccc6ccc(-c7nc(-c8cccc9ccccc89)nc(-c8cccc9c8sc8ccccc89)n7)cc6c5c4c3o2)cc1 ; c1ccc(-c2nc3ccc4oc5ccc6ccc(-c7nc(-c8ccc(-c9ccc%10ccccc%10c9)cc8)nc(-c8ccc9ccccc9c8)n7)cc6c5c4c3o2)cc1]

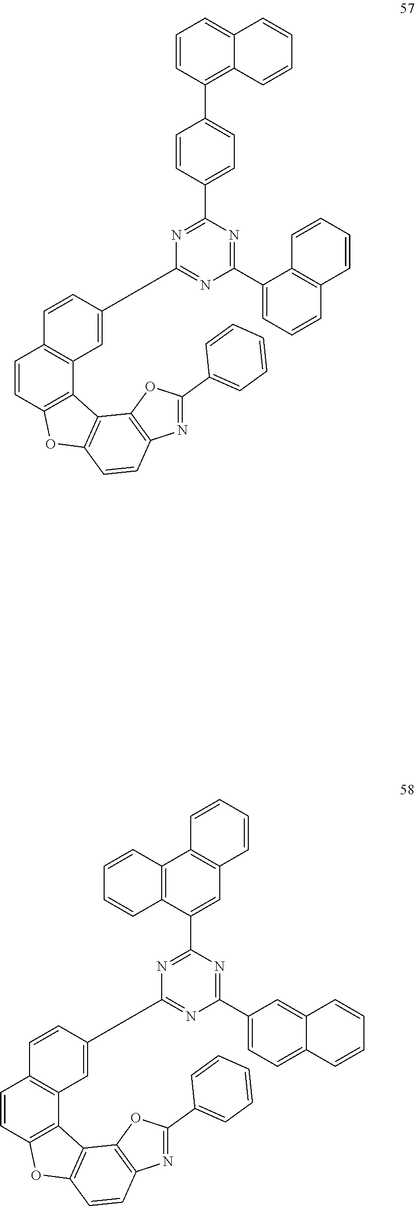 [SMILES: c1ccc(-c2nc3ccc4oc5ccc6ccc(-c7nc(-c8ccc(-c9cccc%10ccccc9%10)cc8)nc(-c8cccc9ccccc89)n7)cc6c5c4c3o2)cc1 ; c1ccc(-c2nc3ccc4oc5ccc6ccc(-c7nc(-c8ccc9ccccc9c8)nc(-c8cc9ccccc9c9ccccc89)n7)cc6c5c4c3o2)cc1]

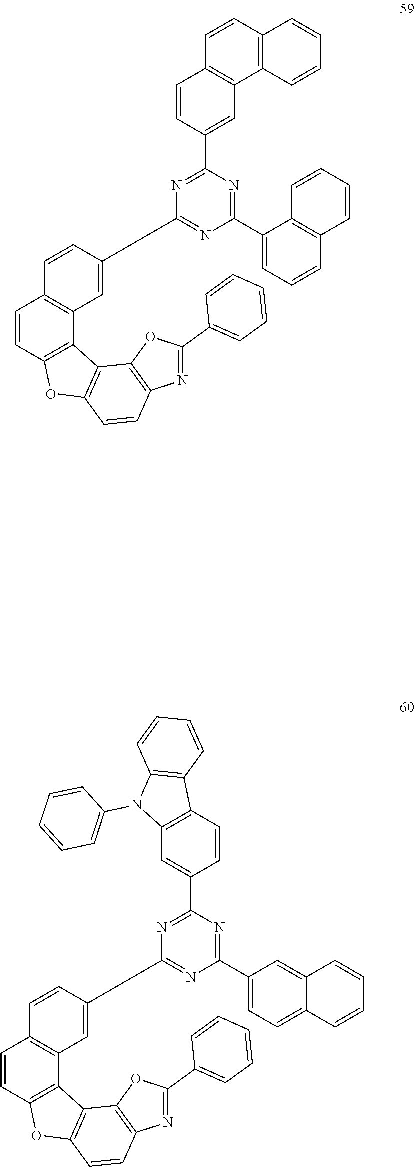 [SMILES: c1ccc(-c2nc3ccc4oc5ccc6ccc(-c7nc(-c8ccc9ccc%10ccccc%10c9c8)nc(-c8cccc9ccccc89)n7)cc6c5c4c3o2)cc1 ; c1ccc(-c2nc3ccc4oc5ccc6ccc(-c7nc(-c8ccc9ccccc9c8)nc(-c8ccc9c%10ccccc%10n(-c%10ccccc%10)c9c8)n7)cc6c5c4c3o2)cc1]

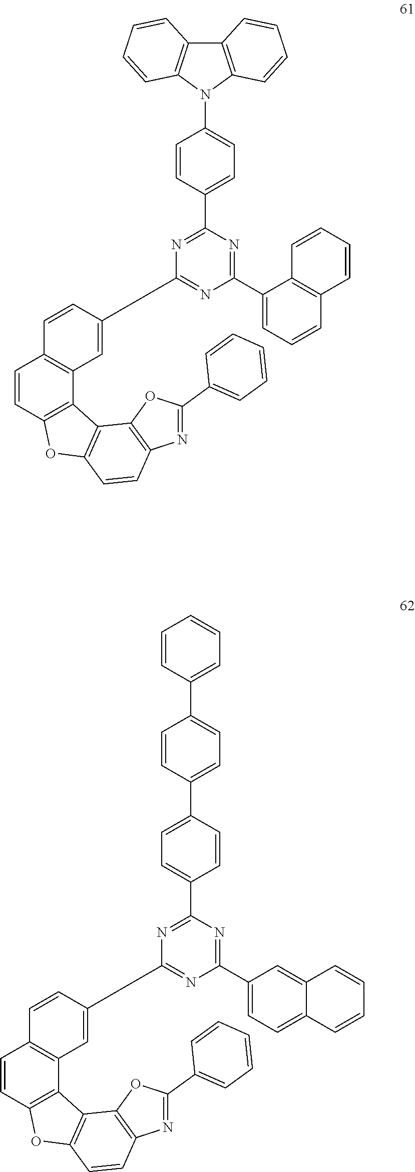 [SMILES: c1ccc(-c2nc3ccc4oc5ccc6ccc(-c7nc(-c8ccc(-n9c%10ccccc%10c%10ccccc%109)cc8)nc(-c8cccc9ccccc89)n7)cc6c5c4c3o2)cc1 ; c1ccc(-c2ccc(-c3ccc(-c4nc(-c5ccc6ccccc6c5)nc(-c5ccc6ccc7oc8ccc9nc(-c%10ccccc%10)oc9c8c7c6c5)n4)cc3)cc2)cc1]

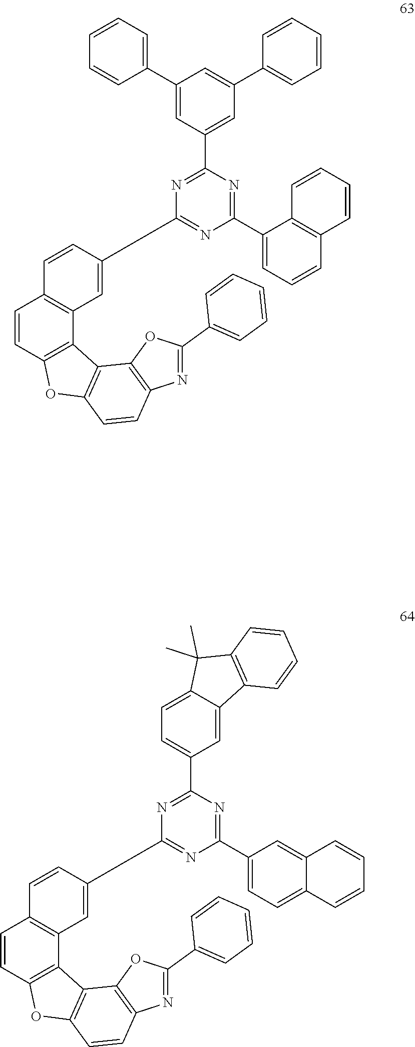 [SMILES: c1ccc(-c2cc(-c3ccccc3)cc(-c3nc(-c4ccc5ccc6oc7ccc8nc(-c9ccccc9)oc8c7c6c5c4)nc(-c4cccc5ccccc45)n3)c2)cc1 ; CC1(C)c2ccccc2-c2cc(-c3nc(-c4ccc5ccccc5c4)nc(-c4ccc5ccc6oc7ccc8nc(-c9ccccc9)oc8c7c6c5c4)n3)ccc21]

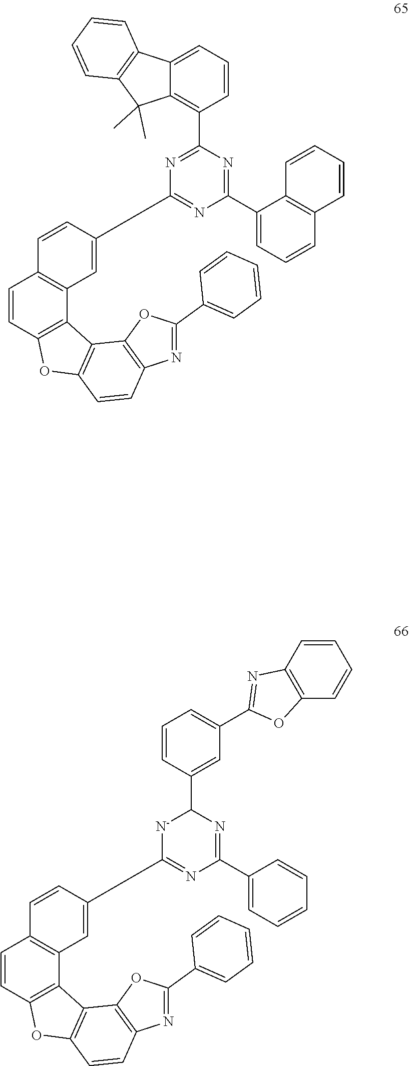 [SMILES: CC1(C)c2ccccc2-c2cccc(-c3nc(-c4ccc5ccc6oc7ccc8nc(-c9ccccc9)oc8c7c6c5c4)nc(-c4cccc5ccccc45)n3)c21 ; c1ccc(C2=NC(c3cccc(-c4nc5ccccc5o4)c3)[N-]C(c3ccc4ccc5oc6ccc7nc(-c8ccccc8)oc7c6c5c4c3)=N2)cc1]

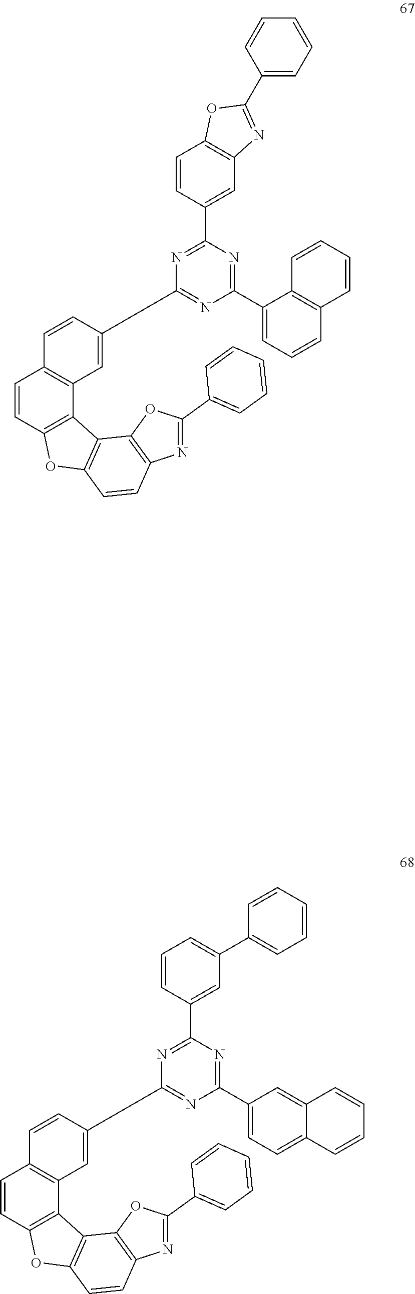 [SMILES: c1ccc(-c2nc3cc(-c4nc(-c5ccc6ccc7oc8ccc9nc(-c%10ccccc%10)oc9c8c7c6c5)nc(-c5cccc6ccccc56)n4)ccc3o2)cc1 ; c1ccc(-c2cccc(-c3nc(-c4ccc5ccccc5c4)nc(-c4ccc5ccc6oc7ccc8nc(-c9ccccc9)oc8c7c6c5c4)n3)c2)cc1]

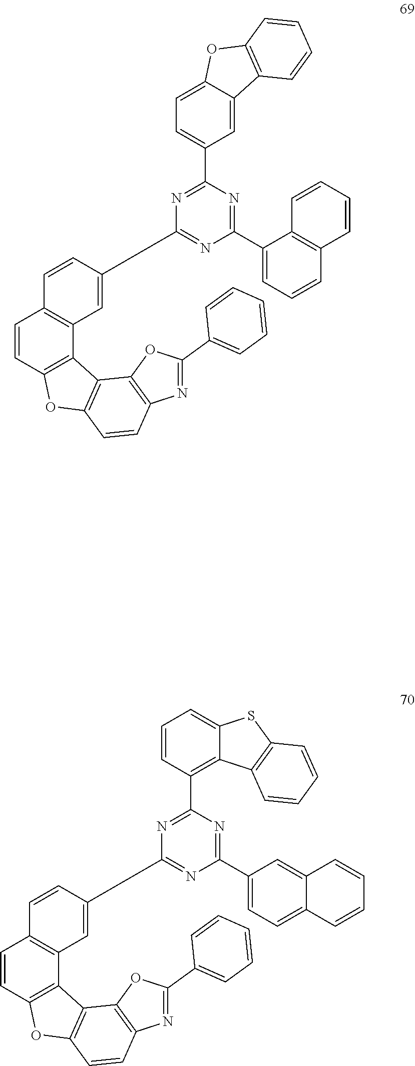 [SMILES: c1ccc(-c2nc3ccc4oc5ccc6ccc(-c7nc(-c8ccc9oc%10ccccc%10c9c8)nc(-c8cccc9ccccc89)n7)cc6c5c4c3o2)cc1 ; c1ccc(-c2nc3ccc4oc5ccc6ccc(-c7nc(-c8ccc9ccccc9c8)nc(-c8cccc9sc%10ccccc%10c89)n7)cc6c5c4c3o2)cc1]

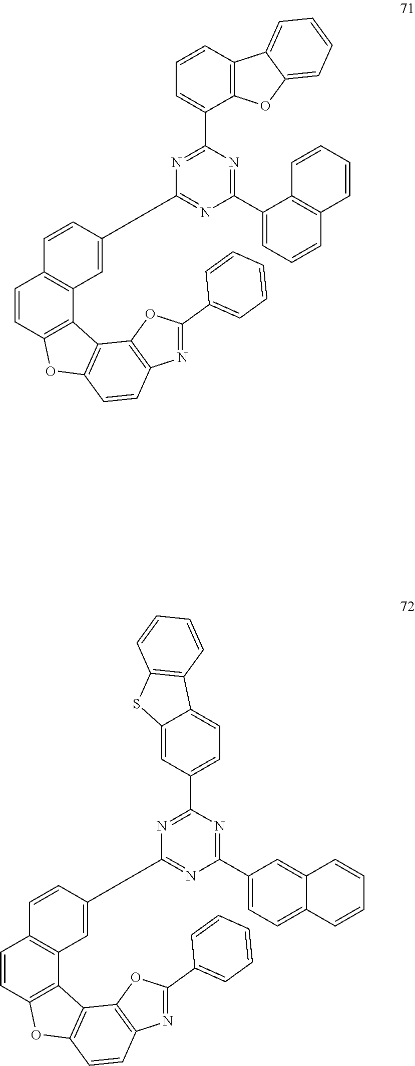 [SMILES: c1ccc(-c2nc3ccc4oc5ccc6ccc(-c7nc(-c8cccc9ccccc89)nc(-c8cccc9c8oc8ccccc89)n7)cc6c5c4c3o2)cc1 ; c1ccc(-c2nc3ccc4oc5ccc6ccc(-c7nc(-c8ccc9ccccc9c8)nc(-c8ccc9c(c8)sc8ccccc89)n7)cc6c5c4c3o2)cc1]

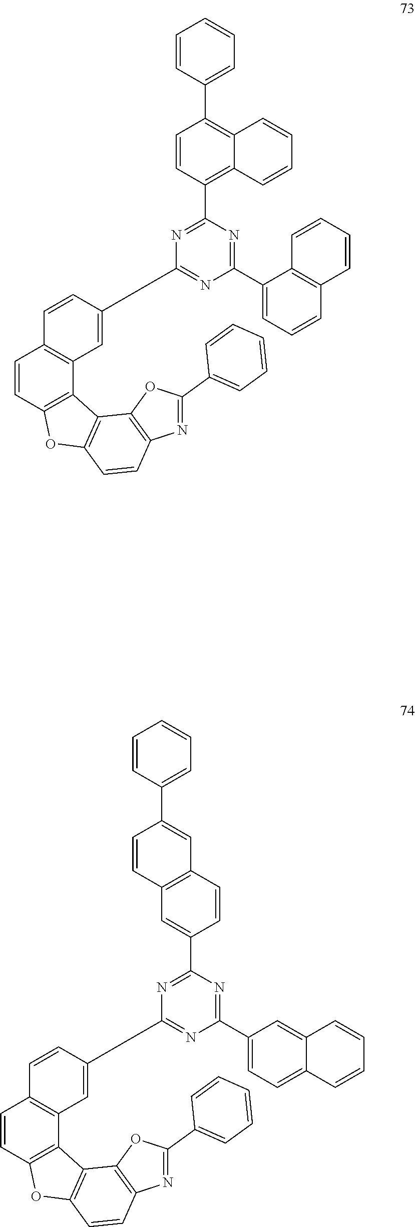 [SMILES: c1ccc(-c2nc3ccc4oc5ccc6ccc(-c7nc(-c8cccc9ccccc89)nc(-c8ccc(-c9ccccc9)c9ccccc89)n7)cc6c5c4c3o2)cc1 ; c1ccc(-c2ccc3cc(-c4nc(-c5ccc6ccccc6c5)nc(-c5ccc6ccc7oc8ccc9nc(-c%10ccccc%10)oc9c8c7c6c5)n4)ccc3c2)cc1]

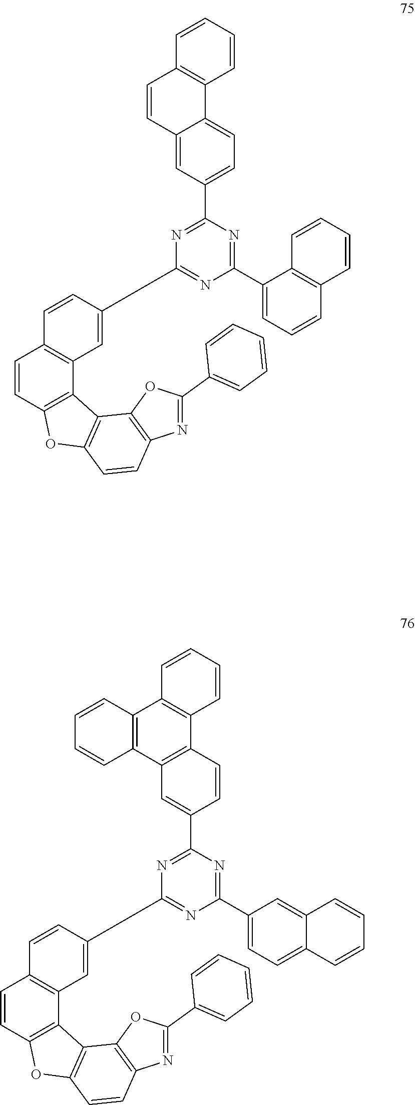 [SMILES: c1ccc(-c2nc3ccc4oc5ccc6ccc(-c7nc(-c8ccc9c(ccc%10ccccc%109)c8)nc(-c8cccc9ccccc89)n7)cc6c5c4c3o2)cc1 ; c1ccc(-c2nc3ccc4oc5ccc6ccc(-c7nc(-c8ccc9ccccc9c8)nc(-c8ccc9c%10ccccc%10c%10ccccc%10c9c8)n7)cc6c5c4c3o2)cc1]

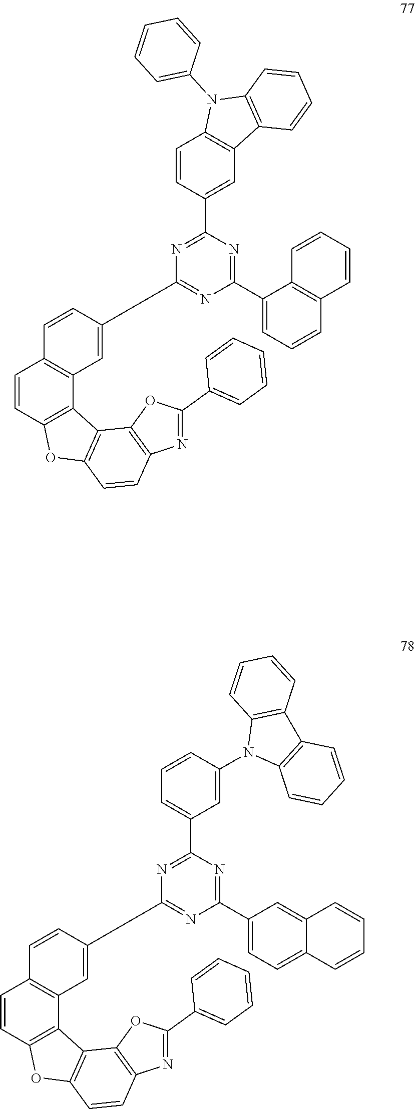 [SMILES: c1ccc(-c2nc3ccc4oc5ccc6ccc(-c7nc(-c8ccc9c(c8)c8ccccc8n9-c8ccccc8)nc(-c8cccc9ccccc89)n7)cc6c5c4c3o2)cc1 ; c1ccc(-c2nc3ccc4oc5ccc6ccc(-c7nc(-c8cccc(-n9c%10ccccc%10c%10ccccc%109)c8)nc(-c8ccc9ccccc9c8)n7)cc6c5c4c3o2)cc1]

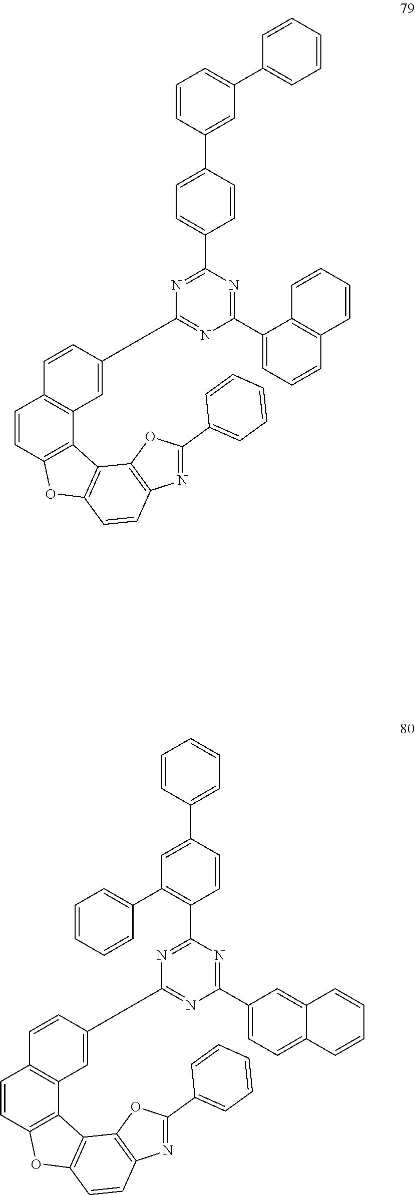 [SMILES: c1ccc(-c2cccc(-c3ccc(-c4nc(-c5ccc6ccc7oc8ccc9nc(-c%10ccccc%10)oc9c8c7c6c5)nc(-c5cccc6ccccc56)n4)cc3)c2)cc1 ; c1ccc(-c2ccc(-c3nc(-c4ccc5ccccc5c4)nc(-c4ccc5ccc6oc7ccc8nc(-c9ccccc9)oc8c7c6c5c4)n3)c(-c3ccccc3)c2)cc1]

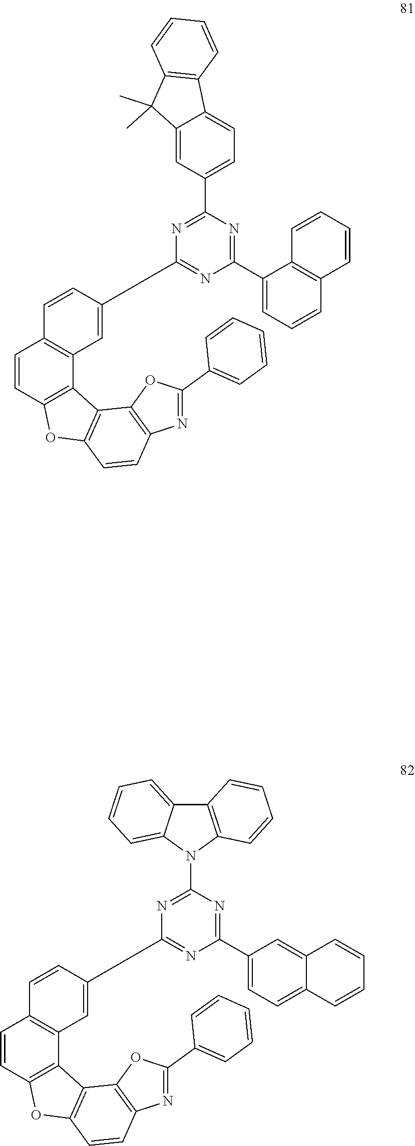 [SMILES: CC1(C)c2ccccc2-c2ccc(-c3nc(-c4ccc5ccc6oc7ccc8nc(-c9ccccc9)oc8c7c6c5c4)nc(-c4cccc5ccccc45)n3)cc21 ; c1ccc(-c2nc3ccc4oc5ccc6ccc(-c7nc(-c8ccc9ccccc9c8)nc(-n8c9ccccc9c9ccccc98)n7)cc6c5c4c3o2)cc1]

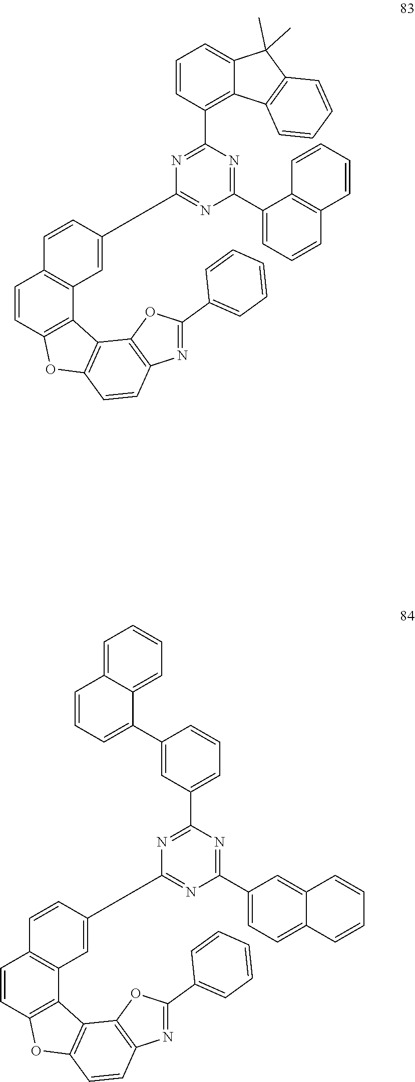 [SMILES: CC1(C)c2ccccc2-c2c(-c3nc(-c4ccc5ccc6oc7ccc8nc(-c9ccccc9)oc8c7c6c5c4)nc(-c4cccc5ccccc45)n3)cccc21 ; c1ccc(-c2nc3ccc4oc5ccc6ccc(-c7nc(-c8cccc(-c9cccc%10ccccc9%10)c8)nc(-c8ccc9ccccc9c8)n7)cc6c5c4c3o2)cc1]

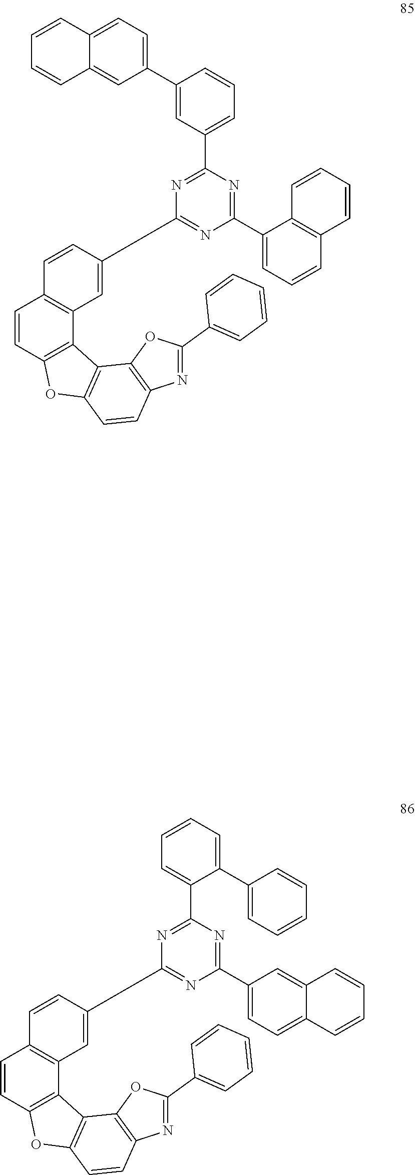 [SMILES: c1ccc(-c2nc3ccc4oc5ccc6ccc(-c7nc(-c8cccc(-c9ccc%10ccccc%10c9)c8)nc(-c8cccc9ccccc89)n7)cc6c5c4c3o2)cc1 ; c1ccc(-c2nc3ccc4oc5ccc6ccc(-c7nc(-c8ccc9ccccc9c8)nc(-c8ccccc8-c8ccccc8)n7)cc6c5c4c3o2)cc1]

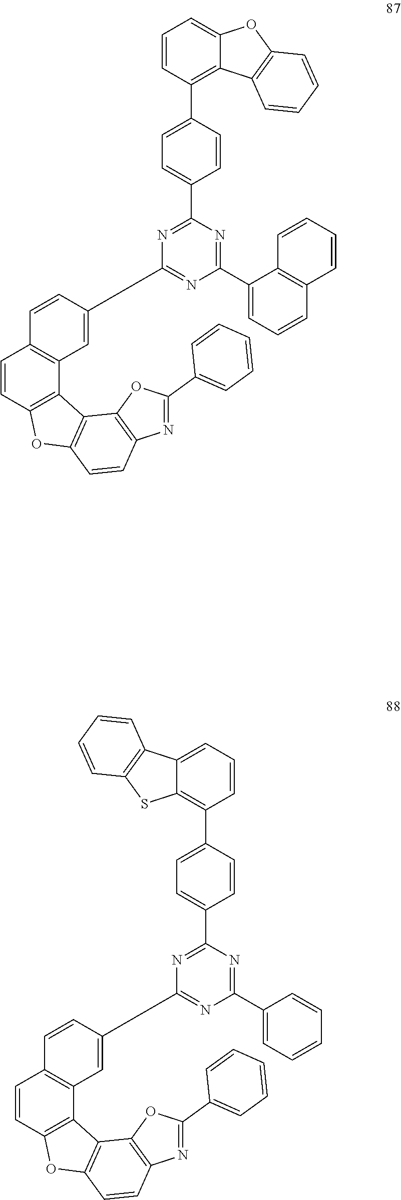 [SMILES: c1ccc(-c2nc3ccc4oc5ccc6ccc(-c7nc(-c8ccc(-c9cccc%10oc%11ccccc%11c9%10)cc8)nc(-c8cccc9ccccc89)n7)cc6c5c4c3o2)cc1 ; c1ccc(-c2nc(-c3ccc(-c4cccc5c4sc4ccccc45)cc3)nc(-c3ccc4ccc5oc6ccc7nc(-c8ccccc8)oc7c6c5c4c3)n2)cc1]

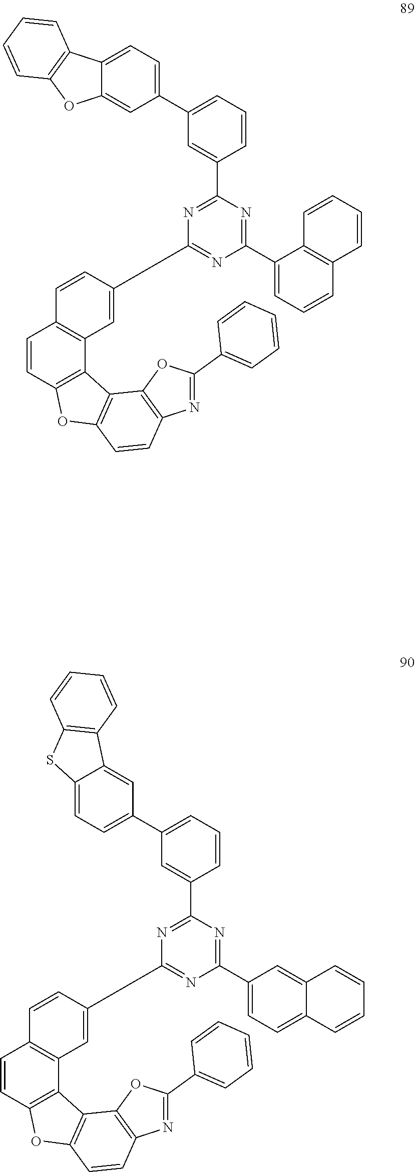 [SMILES: c1ccc(-c2nc3ccc4oc5ccc6ccc(-c7nc(-c8cccc(-c9ccc%10c(c9)oc9ccccc9%10)c8)nc(-c8cccc9ccccc89)n7)cc6c5c4c3o2)cc1 ; c1ccc(-c2nc3ccc4oc5ccc6ccc(-c7nc(-c8cccc(-c9ccc%10sc%11ccccc%11c%10c9)c8)nc(-c8ccc9ccccc9c8)n7)cc6c5c4c3o2)cc1]

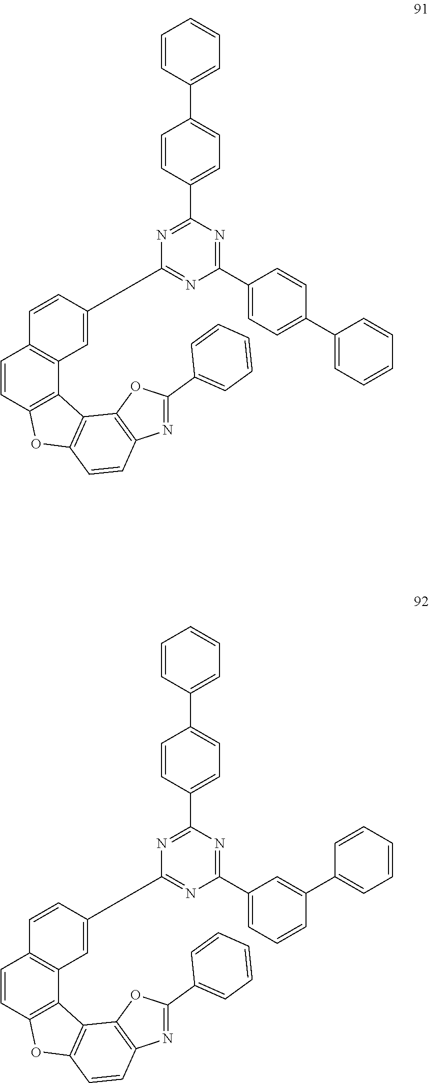 [SMILES: c1ccc(-c2ccc(-c3nc(-c4ccc(-c5ccccc5)cc4)nc(-c4ccc5ccc6oc7ccc8nc(-c9ccccc9)oc8c7c6c5c4)n3)cc2)cc1 ; c1ccc(-c2ccc(-c3nc(-c4cccc(-c5ccccc5)c4)nc(-c4ccc5ccc6oc7ccc8nc(-c9ccccc9)oc8c7c6c5c4)n3)cc2)cc1]

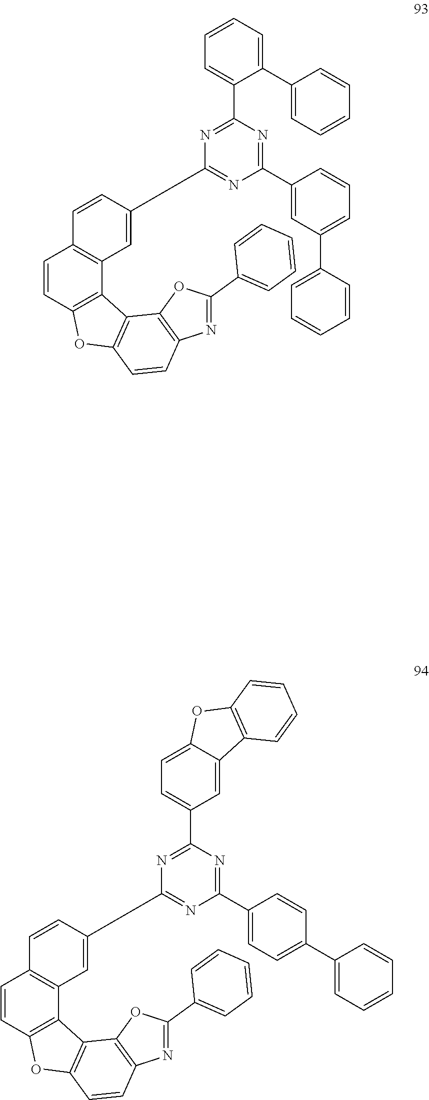 [SMILES: c1ccc(-c2cccc(-c3nc(-c4ccc5ccc6oc7ccc8nc(-c9ccccc9)oc8c7c6c5c4)nc(-c4ccccc4-c4ccccc4)n3)c2)cc1 ; c1ccc(-c2ccc(-c3nc(-c4ccc5oc6ccccc6c5c4)nc(-c4ccc5ccc6oc7ccc8nc(-c9ccccc9)oc8c7c6c5c4)n3)cc2)cc1]

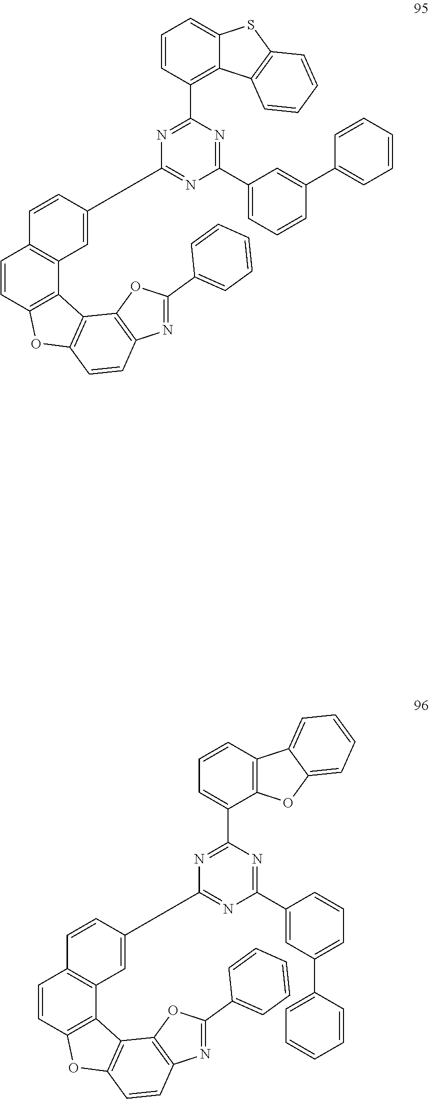 [SMILES: c1ccc(-c2cccc(-c3nc(-c4ccc5ccc6oc7ccc8nc(-c9ccccc9)oc8c7c6c5c4)nc(-c4cccc5sc6ccccc6c45)n3)c2)cc1 ; c1ccc(-c2cccc(-c3nc(-c4ccc5ccc6oc7ccc8nc(-c9ccccc9)oc8c7c6c5c4)nc(-c4cccc5c4oc4ccccc45)n3)c2)cc1]

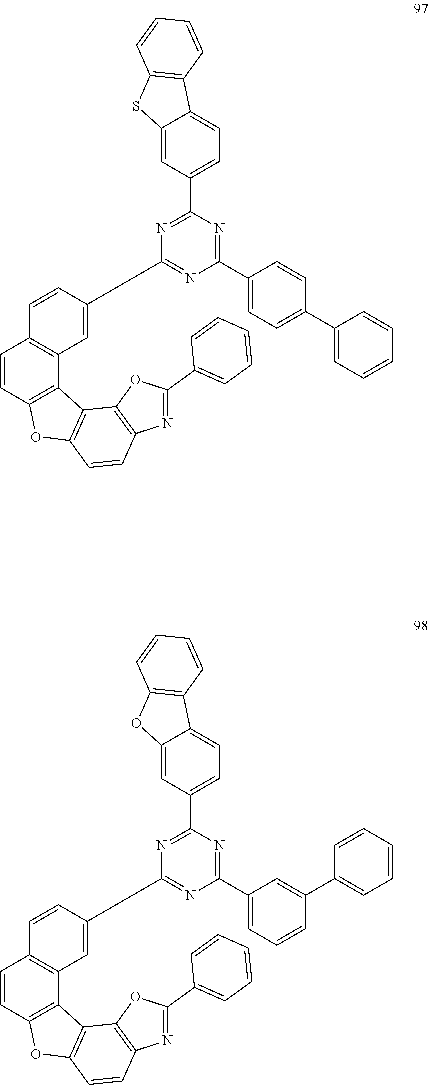 [SMILES: c1ccc(-c2ccc(-c3nc(-c4ccc5c(c4)sc4ccccc45)nc(-c4ccc5ccc6oc7ccc8nc(-c9ccccc9)oc8c7c6c5c4)n3)cc2)cc1 ; c1ccc(-c2cccc(-c3nc(-c4ccc5c(c4)oc4ccccc45)nc(-c4ccc5ccc6oc7ccc8nc(-c9ccccc9)oc8c7c6c5c4)n3)c2)cc1]

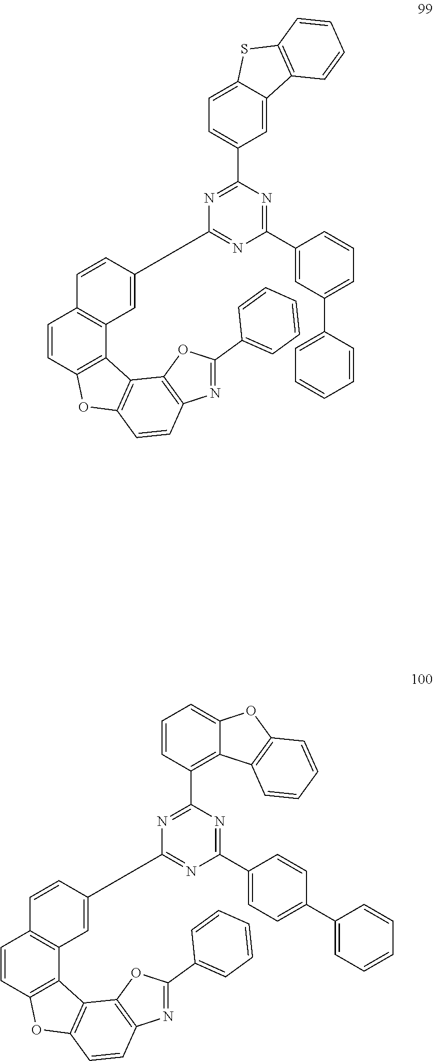 [SMILES: c1ccc(-c2cccc(-c3nc(-c4ccc5sc6ccccc6c5c4)nc(-c4ccc5ccc6oc7ccc8nc(-c9ccccc9)oc8c7c6c5c4)n3)c2)cc1 ; c1ccc(-c2ccc(-c3nc(-c4ccc5ccc6oc7ccc8nc(-c9ccccc9)oc8c7c6c5c4)nc(-c4cccc5oc6ccccc6c45)n3)cc2)cc1]

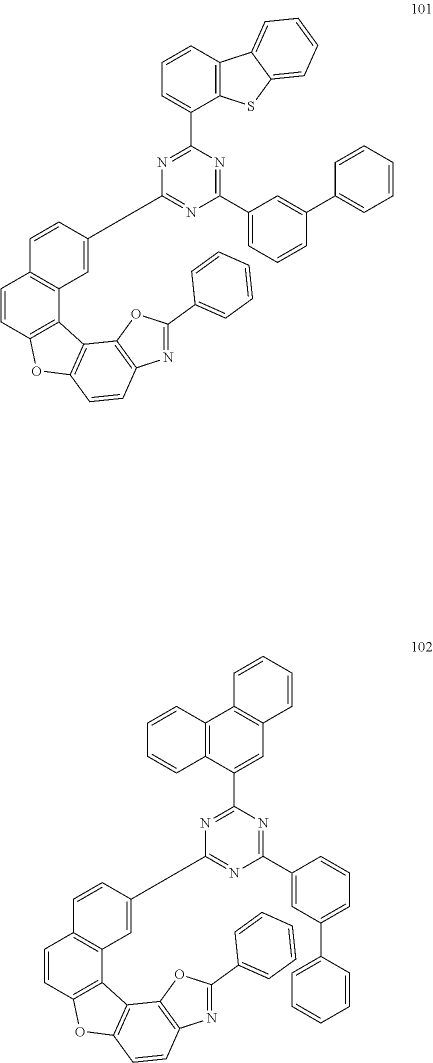 [SMILES: c1ccc(-c2cccc(-c3nc(-c4ccc5ccc6oc7ccc8nc(-c9ccccc9)oc8c7c6c5c4)nc(-c4cccc5c4sc4ccccc45)n3)c2)cc1 ; c1ccc(-c2cccc(-c3nc(-c4ccc5ccc6oc7ccc8nc(-c9ccccc9)oc8c7c6c5c4)nc(-c4cc5ccccc5c5ccccc45)n3)c2)cc1]

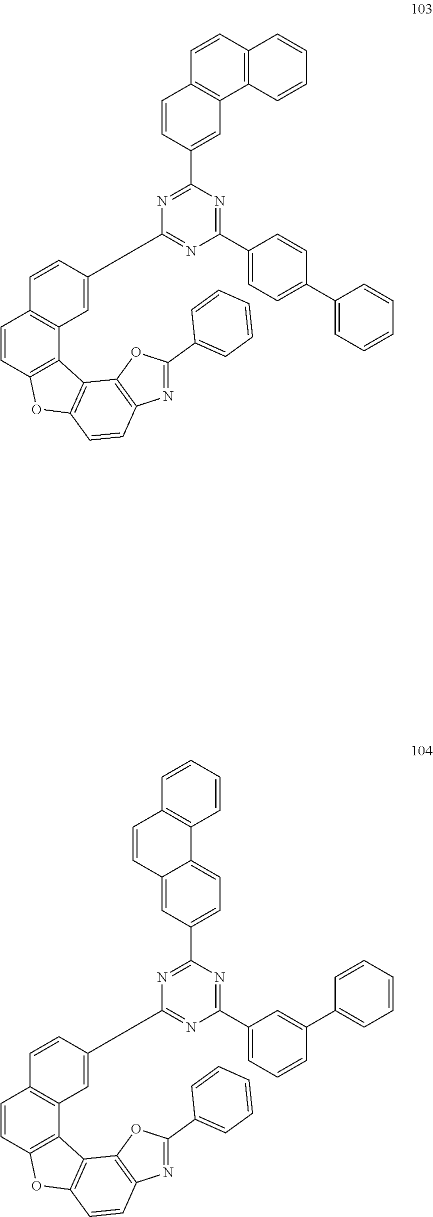 [SMILES: c1ccc(-c2ccc(-c3nc(-c4ccc5ccc6ccccc6c5c4)nc(-c4ccc5ccc6oc7ccc8nc(-c9ccccc9)oc8c7c6c5c4)n3)cc2)cc1 ; c1ccc(-c2cccc(-c3nc(-c4ccc5c(ccc6ccccc65)c4)nc(-c4ccc5ccc6oc7ccc8nc(-c9ccccc9)oc8c7c6c5c4)n3)c2)cc1]

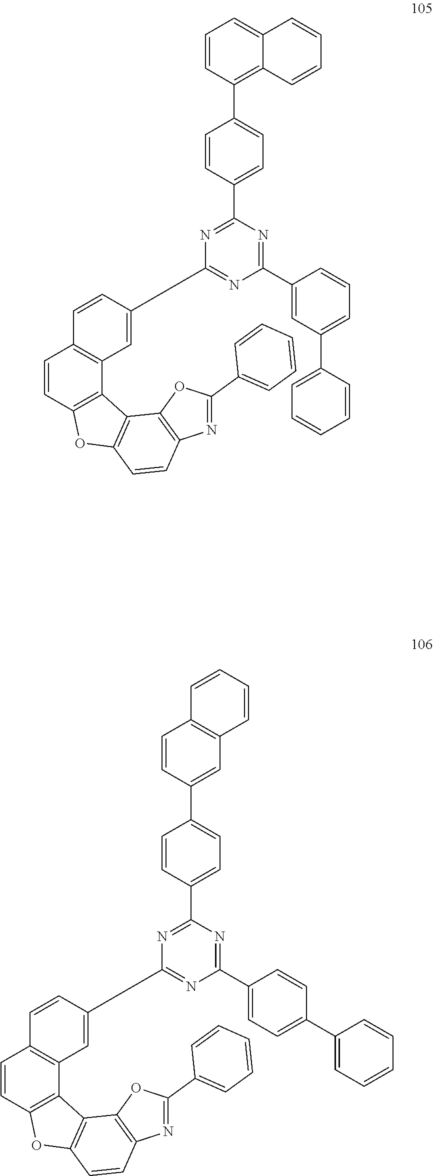 [SMILES: c1ccc(-c2cccc(-c3nc(-c4ccc(-c5cccc6ccccc56)cc4)nc(-c4ccc5ccc6oc7ccc8nc(-c9ccccc9)oc8c7c6c5c4)n3)c2)cc1 ; c1ccc(-c2ccc(-c3nc(-c4ccc(-c5ccc6ccccc6c5)cc4)nc(-c4ccc5ccc6oc7ccc8nc(-c9ccccc9)oc8c7c6c5c4)n3)cc2)cc1]

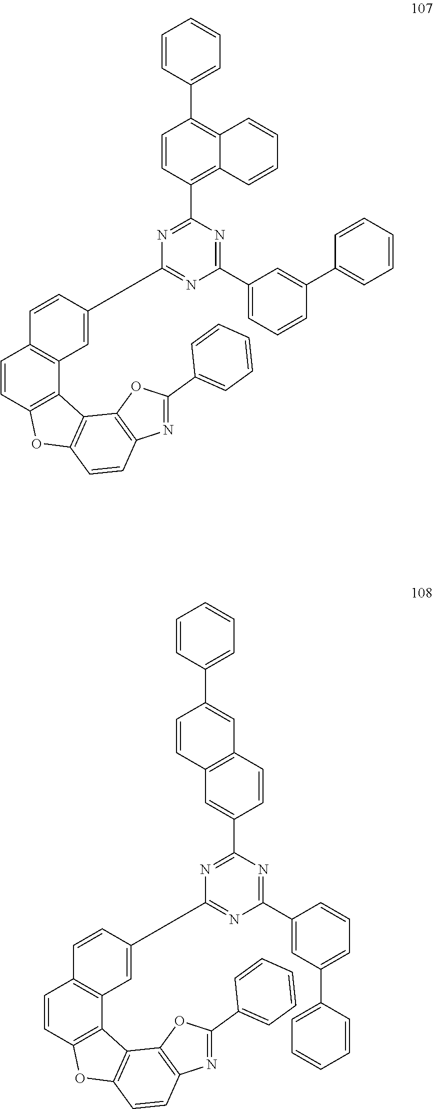 [SMILES: c1ccc(-c2cccc(-c3nc(-c4ccc5ccc6oc7ccc8nc(-c9ccccc9)oc8c7c6c5c4)nc(-c4ccc(-c5ccccc5)c5ccccc45)n3)c2)cc1 ; c1ccc(-c2cccc(-c3nc(-c4ccc5cc(-c6ccccc6)ccc5c4)nc(-c4ccc5ccc6oc7ccc8nc(-c9ccccc9)oc8c7c6c5c4)n3)c2)cc1]

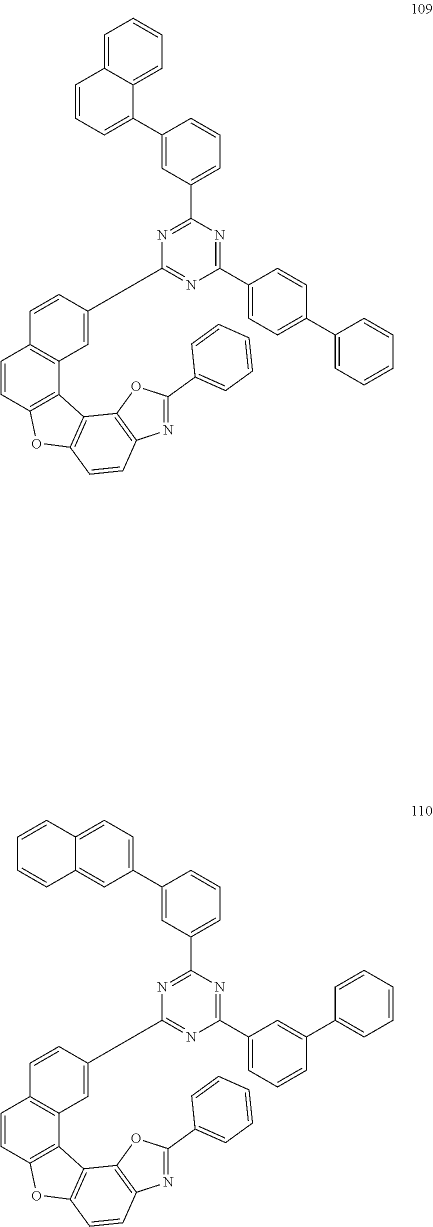 [SMILES: c1ccc(-c2ccc(-c3nc(-c4cccc(-c5cccc6ccccc56)c4)nc(-c4ccc5ccc6oc7ccc8nc(-c9ccccc9)oc8c7c6c5c4)n3)cc2)cc1 ; c1ccc(-c2cccc(-c3nc(-c4cccc(-c5ccc6ccccc6c5)c4)nc(-c4ccc5ccc6oc7ccc8nc(-c9ccccc9)oc8c7c6c5c4)n3)c2)cc1]

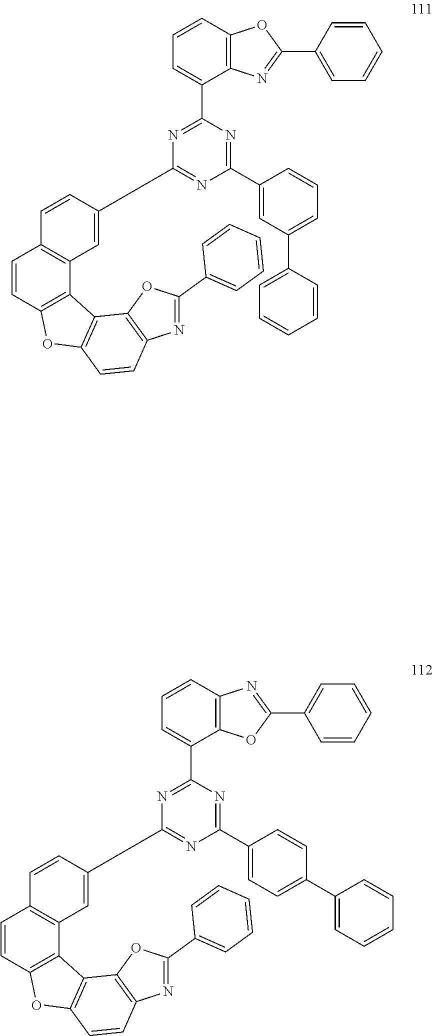 [SMILES: c1ccc(-c2cccc(-c3nc(-c4ccc5ccc6oc7ccc8nc(-c9ccccc9)oc8c7c6c5c4)nc(-c4cccc5oc(-c6ccccc6)nc45)n3)c2)cc1 ; c1ccc(-c2ccc(-c3nc(-c4ccc5ccc6oc7ccc8nc(-c9ccccc9)oc8c7c6c5c4)nc(-c4cccc5nc(-c6ccccc6)oc45)n3)cc2)cc1]

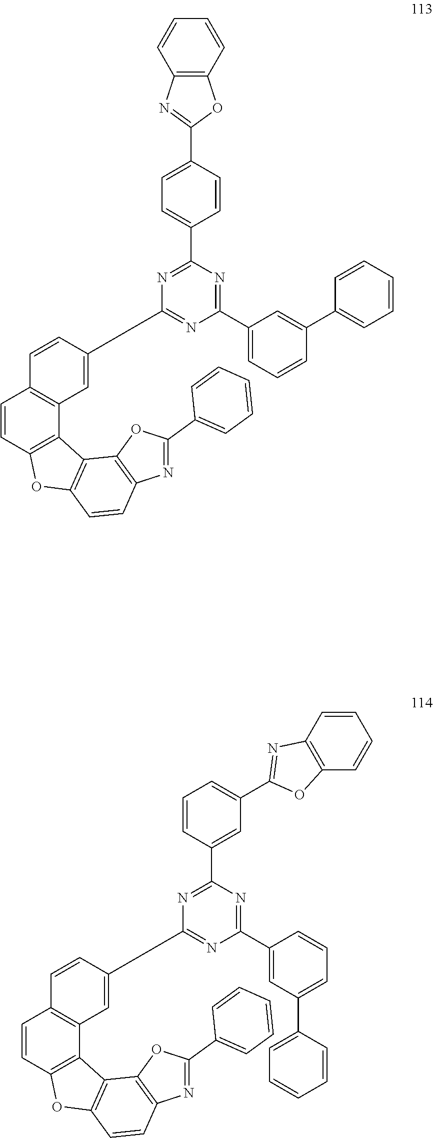 [SMILES: c1ccc(-c2cccc(-c3nc(-c4ccc(-c5nc6ccccc6o5)cc4)nc(-c4ccc5ccc6oc7ccc8nc(-c9ccccc9)oc8c7c6c5c4)n3)c2)cc1 ; c1ccc(-c2cccc(-c3nc(-c4cccc(-c5nc6ccccc6o5)c4)nc(-c4ccc5ccc6oc7ccc8nc(-c9ccccc9)oc8c7c6c5c4)n3)c2)cc1]

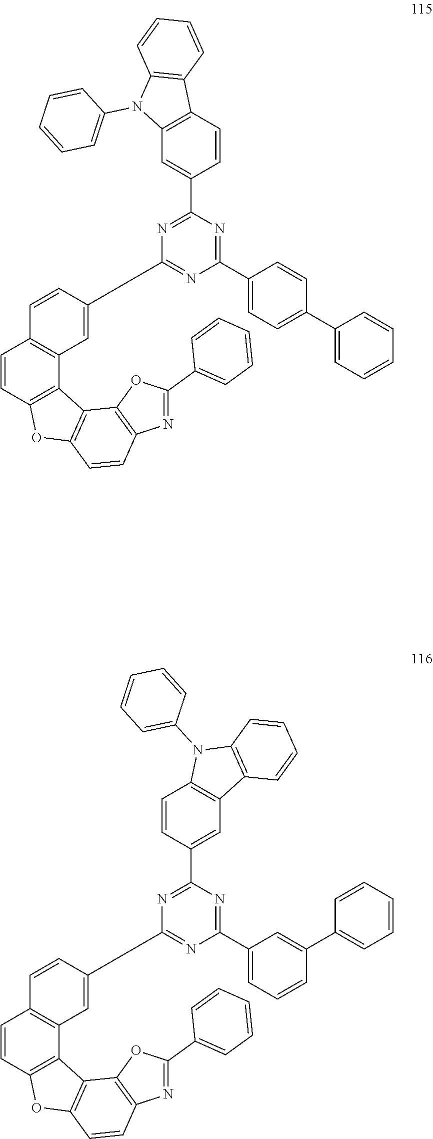 [SMILES: c1ccc(-c2ccc(-c3nc(-c4ccc5ccc6oc7ccc8nc(-c9ccccc9)oc8c7c6c5c4)nc(-c4ccc5c6ccccc6n(-c6ccccc6)c5c4)n3)cc2)cc1 ; c1ccc(-c2cccc(-c3nc(-c4ccc5ccc6oc7ccc8nc(-c9ccccc9)oc8c7c6c5c4)nc(-c4ccc5c(c4)c4ccccc4n5-c4ccccc4)n3)c2)cc1]

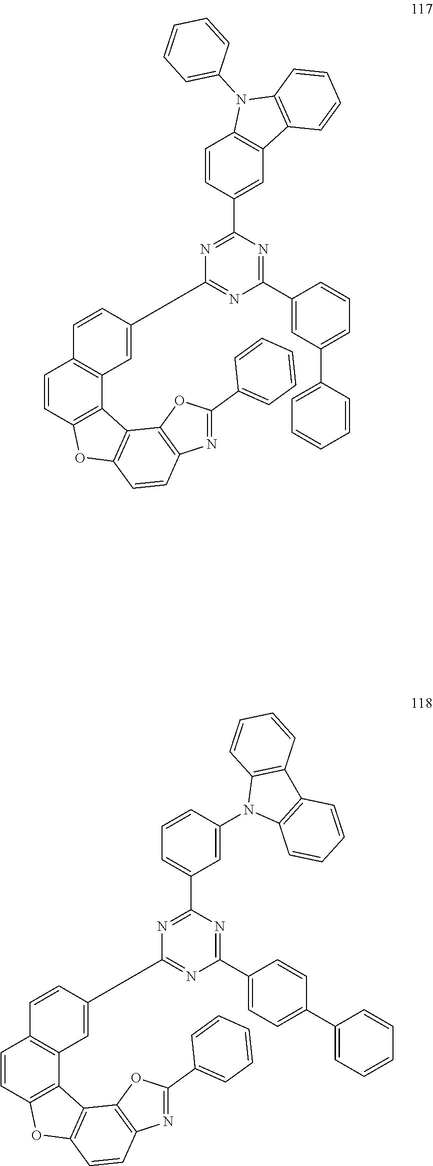 [SMILES: c1ccc(-c2cccc(-c3nc(-c4ccc5ccc6oc7ccc8nc(-c9ccccc9)oc8c7c6c5c4)nc(-c4ccc5c(c4)c4ccccc4n5-c4ccccc4)n3)c2)cc1 ; c1ccc(-c2ccc(-c3nc(-c4cccc(-n5c6ccccc6c6ccccc65)c4)nc(-c4ccc5ccc6oc7ccc8nc(-c9ccccc9)oc8c7c6c5c4)n3)cc2)cc1]

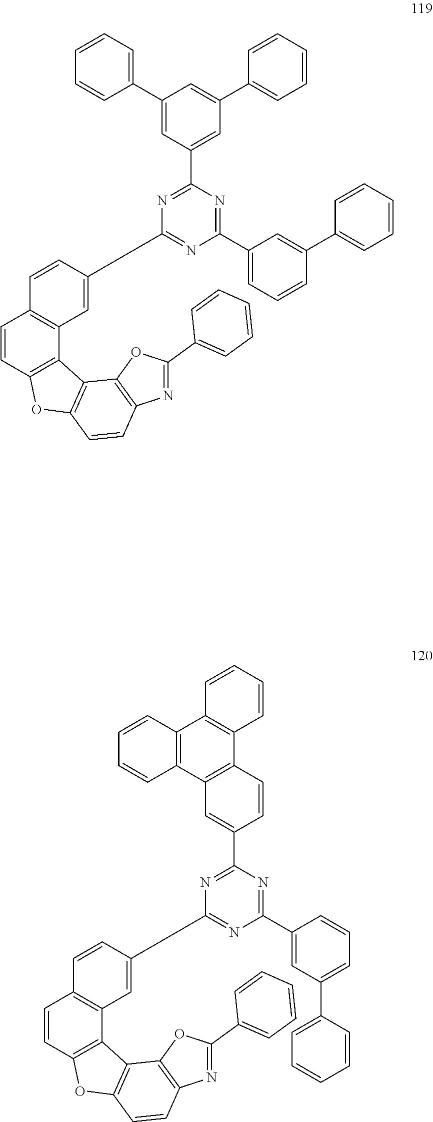 [SMILES: c1ccc(-c2cccc(-c3nc(-c4cc(-c5ccccc5)cc(-c5ccccc5)c4)nc(-c4ccc5ccc6oc7ccc8nc(-c9ccccc9)oc8c7c6c5c4)n3)c2)cc1 ; c1ccc(-c2cccc(-c3nc(-c4ccc5c6ccccc6c6ccccc6c5c4)nc(-c4ccc5ccc6oc7ccc8nc(-c9ccccc9)oc8c7c6c5c4)n3)c2)cc1]

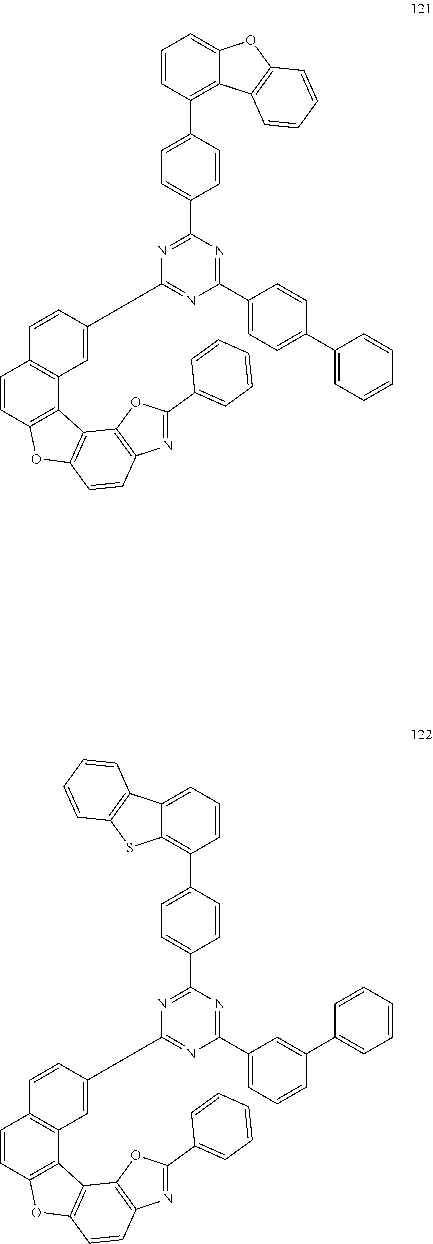 [SMILES: c1ccc(-c2ccc(-c3nc(-c4ccc(-c5cccc6oc7ccccc7c56)cc4)nc(-c4ccc5ccc6oc7ccc8nc(-c9ccccc9)oc8c7c6c5c4)n3)cc2)cc1 ; c1ccc(-c2cccc(-c3nc(-c4ccc(-c5cccc6c5sc5ccccc56)cc4)nc(-c4ccc5ccc6oc7ccc8nc(-c9ccccc9)oc8c7c6c5c4)n3)c2)cc1]

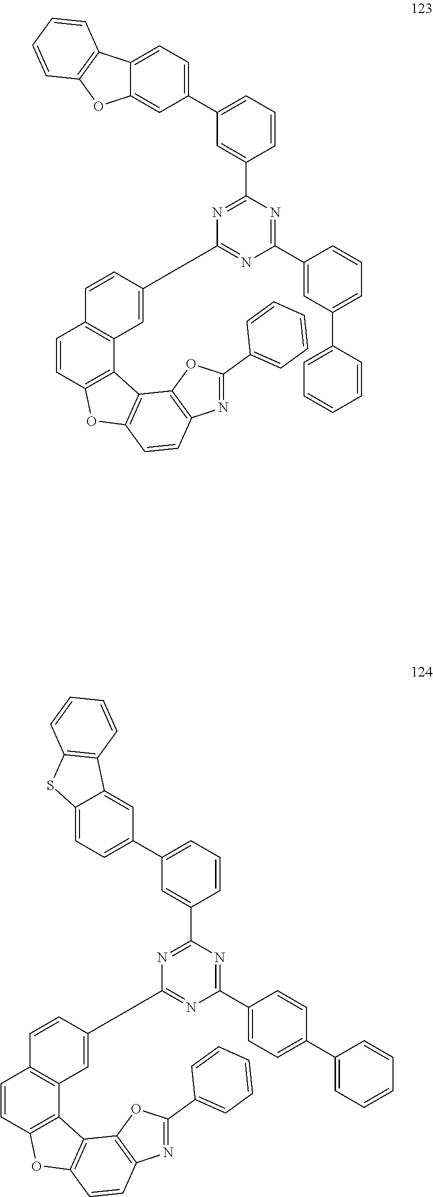 [SMILES: c1ccc(-c2cccc(-c3nc(-c4cccc(-c5ccc6c(c5)oc5ccccc56)c4)nc(-c4ccc5ccc6oc7ccc8nc(-c9ccccc9)oc8c7c6c5c4)n3)c2)cc1 ; c1ccc(-c2ccc(-c3nc(-c4cccc(-c5ccc6sc7ccccc7c6c5)c4)nc(-c4ccc5ccc6oc7ccc8nc(-c9ccccc9)oc8c7c6c5c4)n3)cc2)cc1]

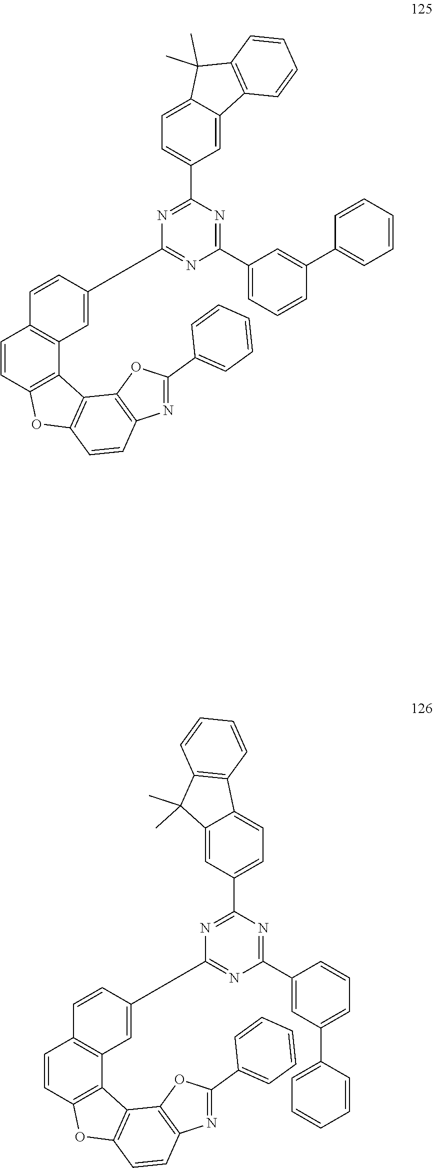 [SMILES: CC1(C)c2ccccc2-c2cc(-c3nc(-c4cccc(-c5ccccc5)c4)nc(-c4ccc5ccc6oc7ccc8nc(-c9ccccc9)oc8c7c6c5c4)n3)ccc21 ; CC1(C)c2ccccc2-c2ccc(-c3nc(-c4cccc(-c5ccccc5)c4)nc(-c4ccc5ccc6oc7ccc8nc(-c9ccccc9)oc8c7c6c5c4)n3)cc21]

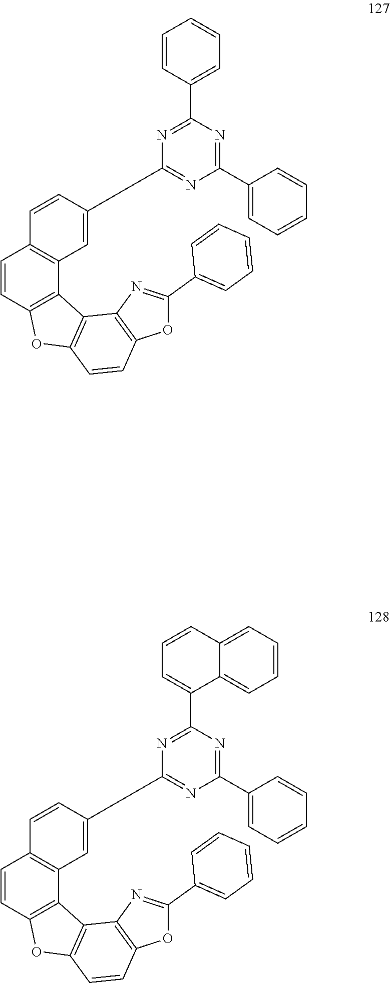 [SMILES: c1ccc(-c2nc(-c3ccccc3)nc(-c3ccc4ccc5oc6ccc7oc(-c8ccccc8)nc7c6c5c4c3)n2)cc1 ; c1ccc(-c2nc(-c3ccc4ccc5oc6ccc7oc(-c8ccccc8)nc7c6c5c4c3)nc(-c3cccc4ccccc34)n2)cc1]

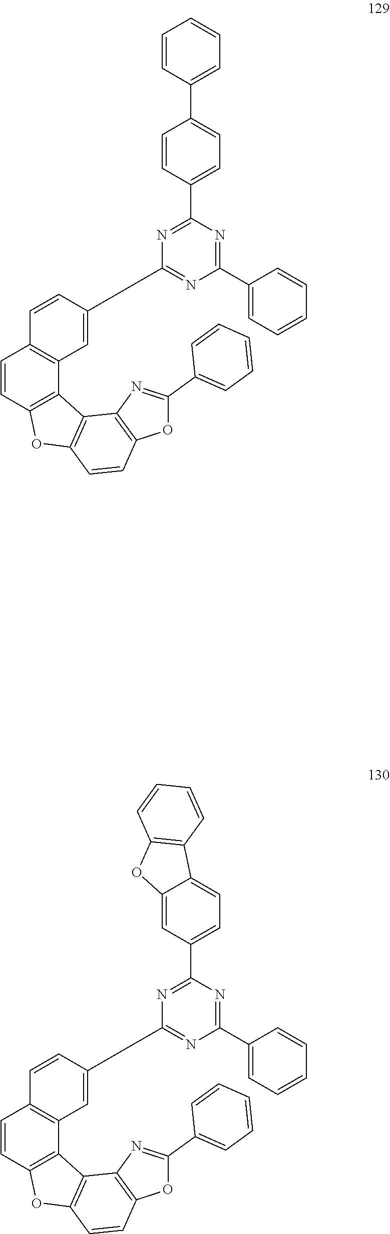 [SMILES: c1ccc(-c2ccc(-c3nc(-c4ccccc4)nc(-c4ccc5ccc6oc7ccc8oc(-c9ccccc9)nc8c7c6c5c4)n3)cc2)cc1 ; c1ccc(-c2nc(-c3ccc4c(c3)oc3ccccc34)nc(-c3ccc4ccc5oc6ccc7oc(-c8ccccc8)nc7c6c5c4c3)n2)cc1]

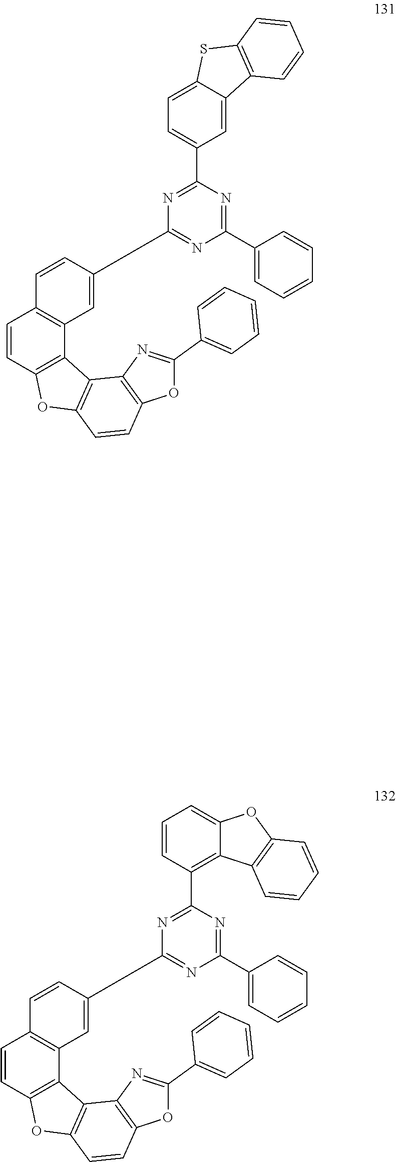 [SMILES: c1ccc(-c2nc(-c3ccc4sc5ccccc5c4c3)nc(-c3ccc4ccc5oc6ccc7oc(-c8ccccc8)nc7c6c5c4c3)n2)cc1 ; c1ccc(-c2nc(-c3ccc4ccc5oc6ccc7oc(-c8ccccc8)nc7c6c5c4c3)nc(-c3cccc4oc5ccccc5c34)n2)cc1]

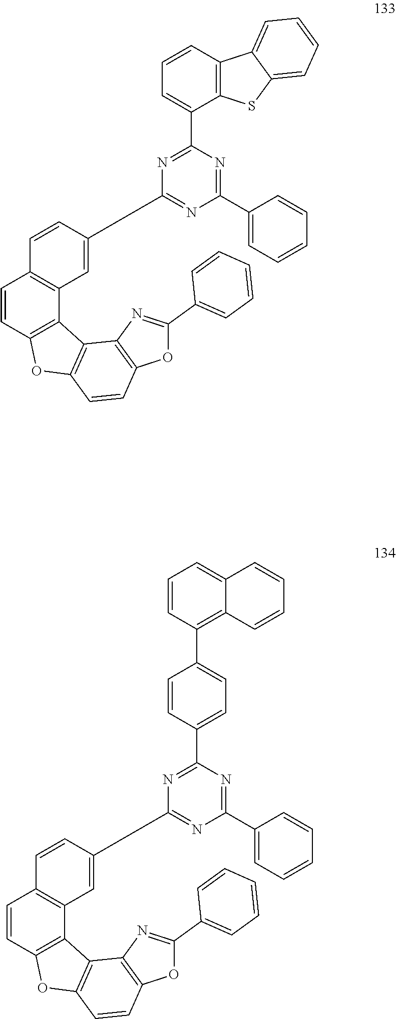 [SMILES: c1ccc(-c2nc(-c3ccc4ccc5oc6ccc7oc(-c8ccccc8)nc7c6c5c4c3)nc(-c3cccc4c3sc3ccccc34)n2)cc1 ; c1ccc(-c2nc(-c3ccc(-c4cccc5ccccc45)cc3)nc(-c3ccc4ccc5oc6ccc7oc(-c8ccccc8)nc7c6c5c4c3)n2)cc1]

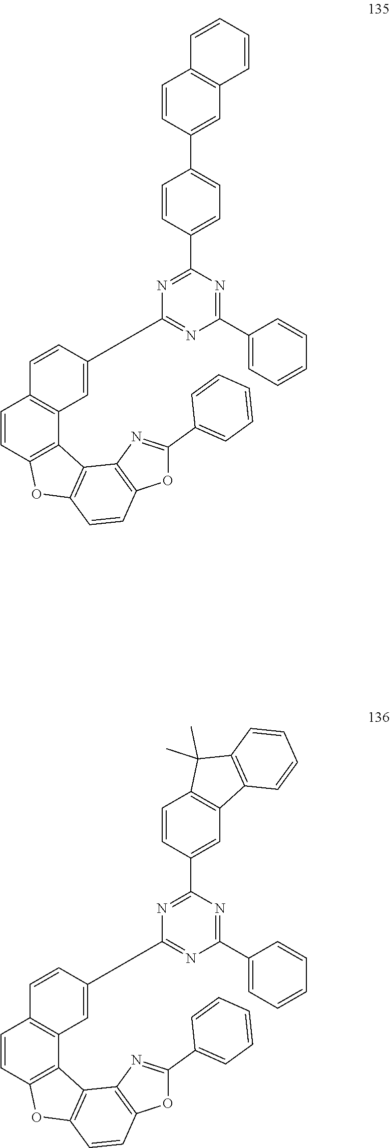 [SMILES: c1ccc(-c2nc(-c3ccc(-c4ccc5ccccc5c4)cc3)nc(-c3ccc4ccc5oc6ccc7oc(-c8ccccc8)nc7c6c5c4c3)n2)cc1 ; CC1(C)c2ccccc2-c2cc(-c3nc(-c4ccccc4)nc(-c4ccc5ccc6oc7ccc8oc(-c9ccccc9)nc8c7c6c5c4)n3)ccc21]

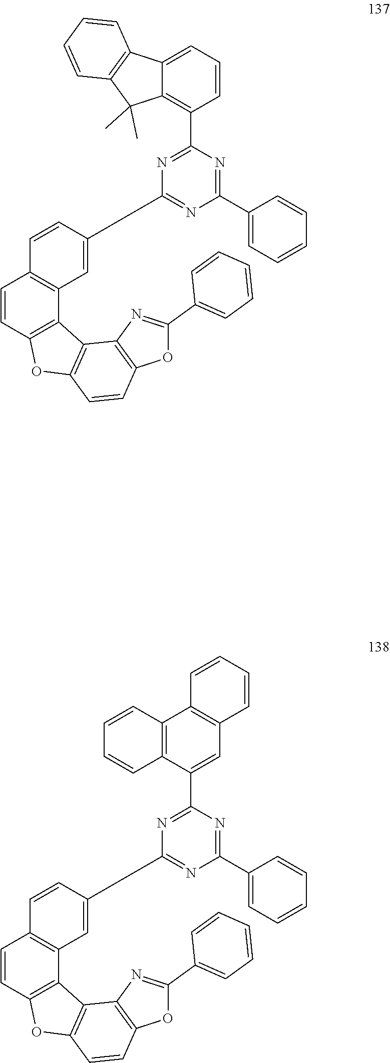 [SMILES: CC1(C)c2ccccc2-c2cccc(-c3nc(-c4ccccc4)nc(-c4ccc5ccc6oc7ccc8oc(-c9ccccc9)nc8c7c6c5c4)n3)c21 ; c1ccc(-c2nc(-c3ccc4ccc5oc6ccc7oc(-c8ccccc8)nc7c6c5c4c3)nc(-c3cc4ccccc4c4ccccc34)n2)cc1]

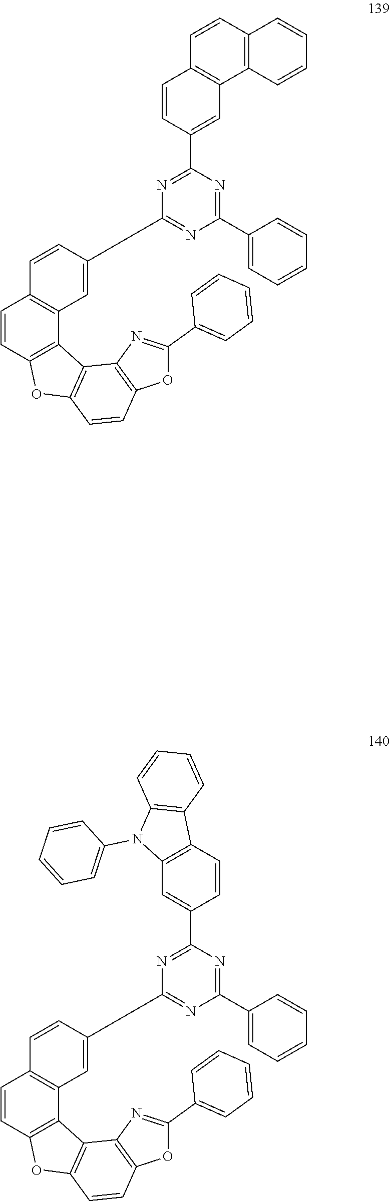 [SMILES: c1ccc(-c2nc(-c3ccc4ccc5ccccc5c4c3)nc(-c3ccc4ccc5oc6ccc7oc(-c8ccccc8)nc7c6c5c4c3)n2)cc1 ; c1ccc(-c2nc(-c3ccc4ccc5oc6ccc7oc(-c8ccccc8)nc7c6c5c4c3)nc(-c3ccc4c5ccccc5n(-c5ccccc5)c4c3)n2)cc1]

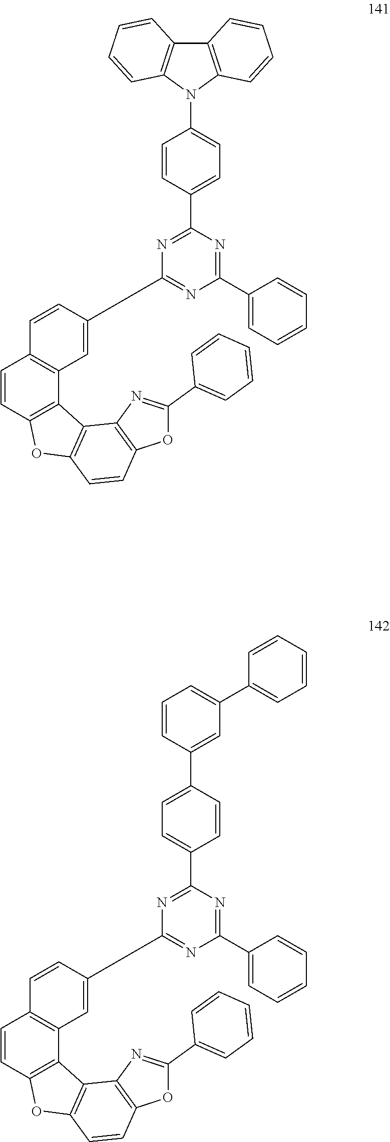 [SMILES: c1ccc(-c2nc(-c3ccc(-n4c5ccccc5c5ccccc54)cc3)nc(-c3ccc4ccc5oc6ccc7oc(-c8ccccc8)nc7c6c5c4c3)n2)cc1 ; c1ccc(-c2cccc(-c3ccc(-c4nc(-c5ccccc5)nc(-c5ccc6ccc7oc8ccc9oc(-c%10ccccc%10)nc9c8c7c6c5)n4)cc3)c2)cc1]

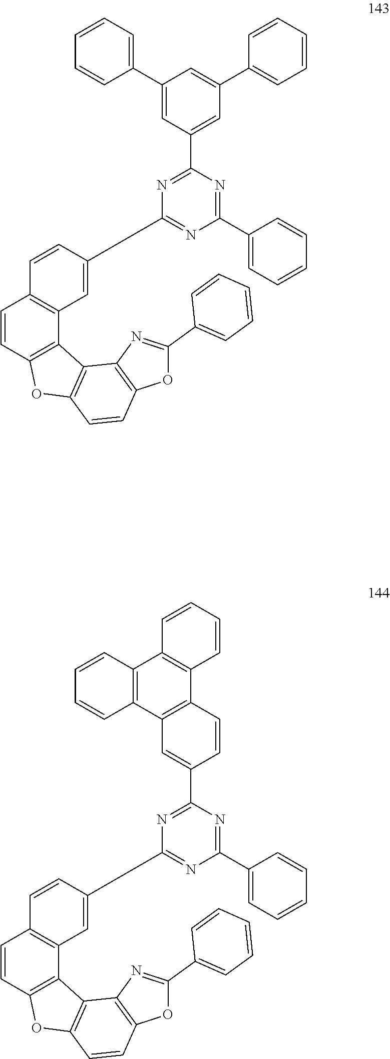 [SMILES: c1ccc(-c2cc(-c3ccccc3)cc(-c3nc(-c4ccccc4)nc(-c4ccc5ccc6oc7ccc8oc(-c9ccccc9)nc8c7c6c5c4)n3)c2)cc1 ; c1ccc(-c2nc(-c3ccc4c5ccccc5c5ccccc5c4c3)nc(-c3ccc4ccc5oc6ccc7oc(-c8ccccc8)nc7c6c5c4c3)n2)cc1]

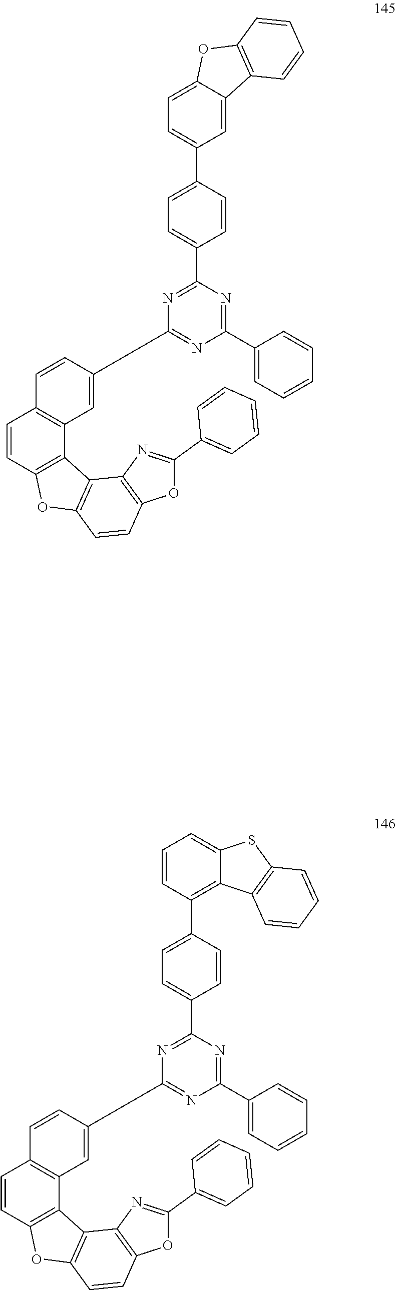 [SMILES: c1ccc(-c2nc(-c3ccc(-c4ccc5oc6ccccc6c5c4)cc3)nc(-c3ccc4ccc5oc6ccc7oc(-c8ccccc8)nc7c6c5c4c3)n2)cc1 ; c1ccc(-c2nc(-c3ccc(-c4cccc5sc6ccccc6c45)cc3)nc(-c3ccc4ccc5oc6ccc7oc(-c8ccccc8)nc7c6c5c4c3)n2)cc1]

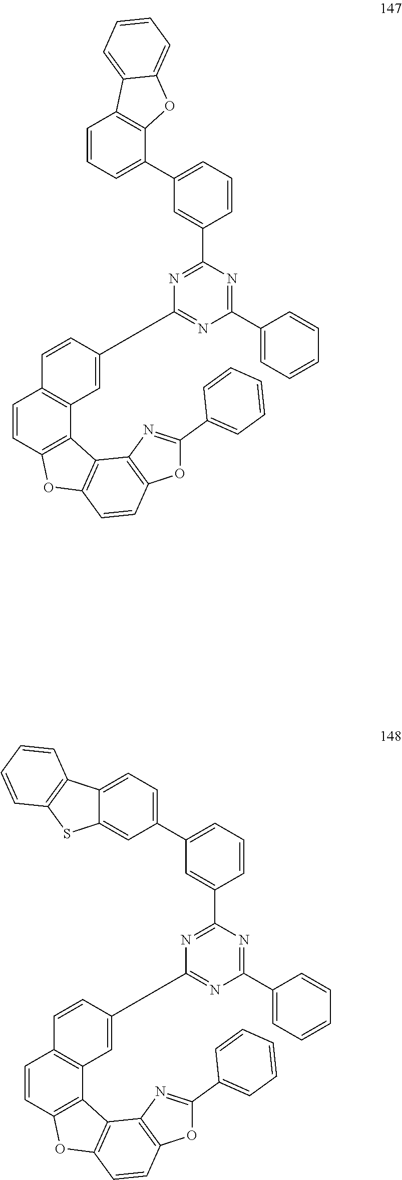 [SMILES: c1ccc(-c2nc(-c3cccc(-c4cccc5c4oc4ccccc45)c3)nc(-c3ccc4ccc5oc6ccc7oc(-c8ccccc8)nc7c6c5c4c3)n2)cc1 ; c1ccc(-c2nc(-c3cccc(-c4ccc5c(c4)sc4ccccc45)c3)nc(-c3ccc4ccc5oc6ccc7oc(-c8ccccc8)nc7c6c5c4c3)n2)cc1]

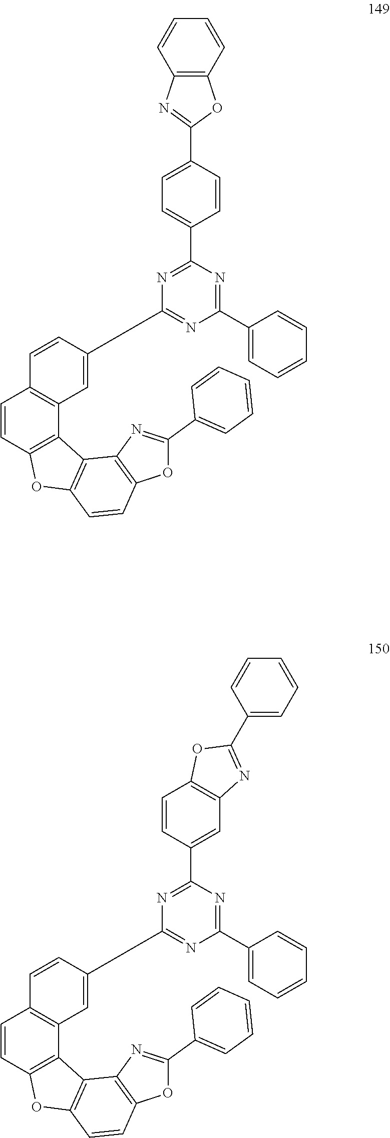 [SMILES: c1ccc(-c2nc(-c3ccc(-c4nc5ccccc5o4)cc3)nc(-c3ccc4ccc5oc6ccc7oc(-c8ccccc8)nc7c6c5c4c3)n2)cc1 ; c1ccc(-c2nc(-c3ccc4oc(-c5ccccc5)nc4c3)nc(-c3ccc4ccc5oc6ccc7oc(-c8ccccc8)nc7c6c5c4c3)n2)cc1]

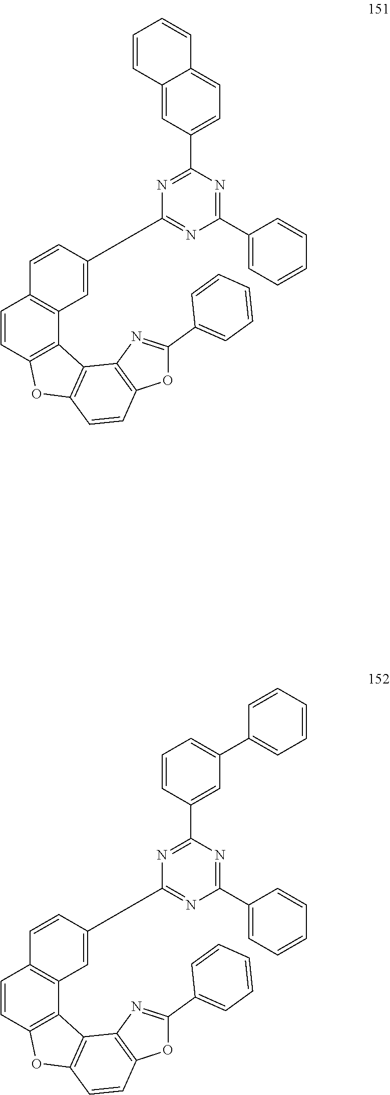 [SMILES: c1ccc(-c2nc(-c3ccc4ccccc4c3)nc(-c3ccc4ccc5oc6ccc7oc(-c8ccccc8)nc7c6c5c4c3)n2)cc1 ; c1ccc(-c2cccc(-c3nc(-c4ccccc4)nc(-c4ccc5ccc6oc7ccc8oc(-c9ccccc9)nc8c7c6c5c4)n3)c2)cc1]

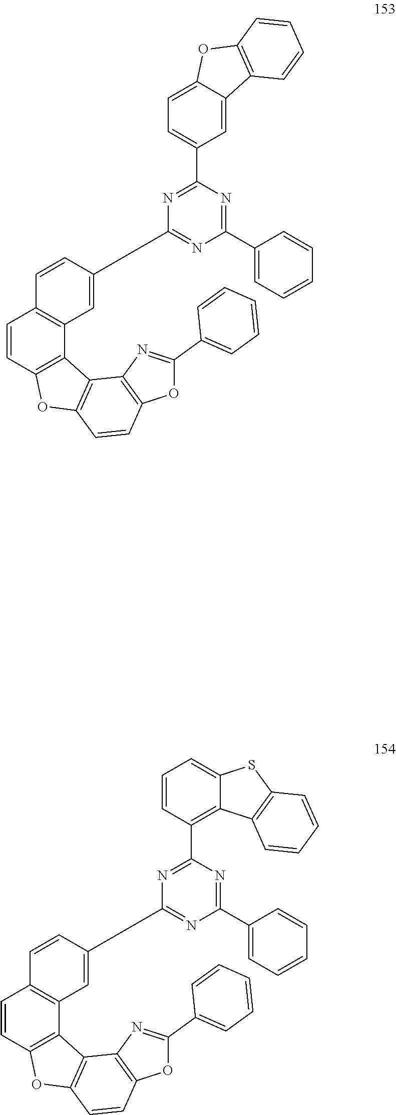 [SMILES: c1ccc(-c2nc(-c3ccc4oc5ccccc5c4c3)nc(-c3ccc4ccc5oc6ccc7oc(-c8ccccc8)nc7c6c5c4c3)n2)cc1 ; c1ccc(-c2nc(-c3ccc4ccc5oc6ccc7oc(-c8ccccc8)nc7c6c5c4c3)nc(-c3cccc4sc5ccccc5c34)n2)cc1]

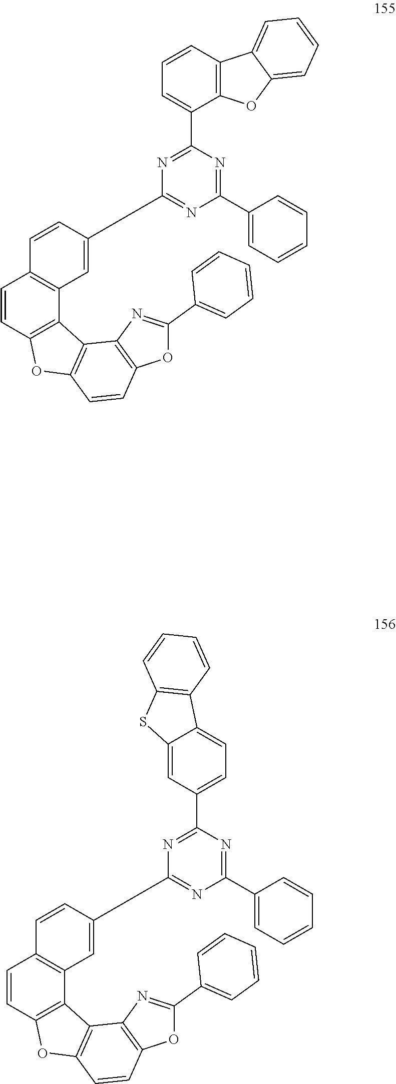 [SMILES: c1ccc(-c2nc(-c3ccc4ccc5oc6ccc7oc(-c8ccccc8)nc7c6c5c4c3)nc(-c3cccc4c3oc3ccccc34)n2)cc1 ; c1ccc(-c2nc(-c3ccc4c(c3)sc3ccccc34)nc(-c3ccc4ccc5oc6ccc7oc(-c8ccccc8)nc7c6c5c4c3)n2)cc1]

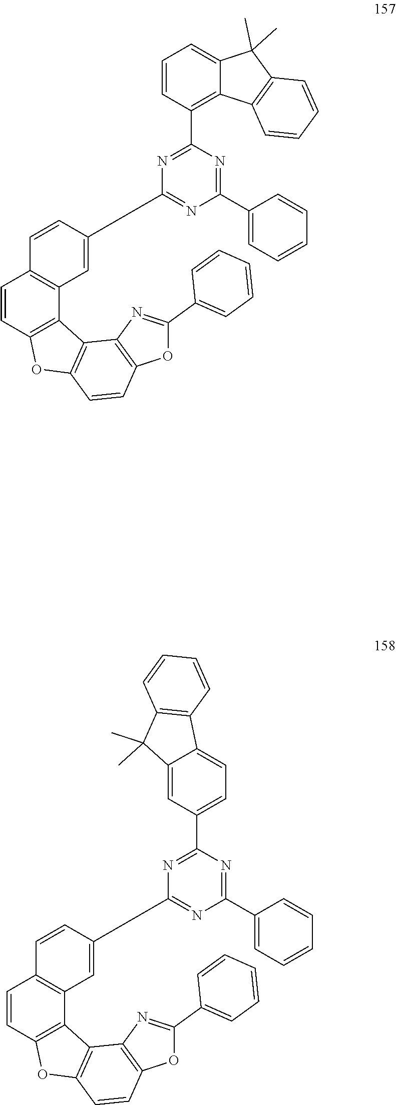 [SMILES: CC1(C)c2ccccc2-c2c(-c3nc(-c4ccccc4)nc(-c4ccc5ccc6oc7ccc8oc(-c9ccccc9)nc8c7c6c5c4)n3)cccc21 ; CC1(C)c2ccccc2-c2ccc(-c3nc(-c4ccccc4)nc(-c4ccc5ccc6oc7ccc8oc(-c9ccccc9)nc8c7c6c5c4)n3)cc21]

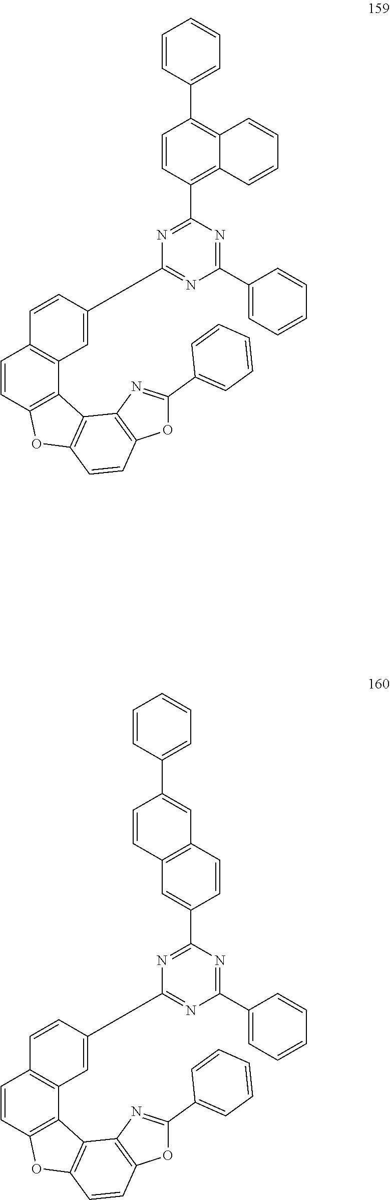 [SMILES: c1ccc(-c2nc(-c3ccc4ccc5oc6ccc7oc(-c8ccccc8)nc7c6c5c4c3)nc(-c3ccc(-c4ccccc4)c4ccccc34)n2)cc1 ; c1ccc(-c2ccc3cc(-c4nc(-c5ccccc5)nc(-c5ccc6ccc7oc8ccc9oc(-c%10ccccc%10)nc9c8c7c6c5)n4)ccc3c2)cc1]

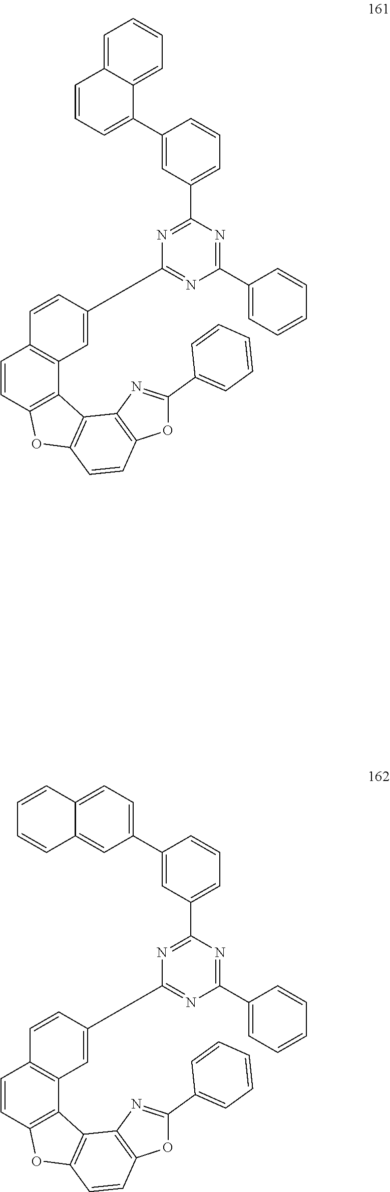 [SMILES: c1ccc(-c2nc(-c3cccc(-c4cccc5ccccc45)c3)nc(-c3ccc4ccc5oc6ccc7oc(-c8ccccc8)nc7c6c5c4c3)n2)cc1 ; c1ccc(-c2nc(-c3cccc(-c4ccc5ccccc5c4)c3)nc(-c3ccc4ccc5oc6ccc7oc(-c8ccccc8)nc7c6c5c4c3)n2)cc1]

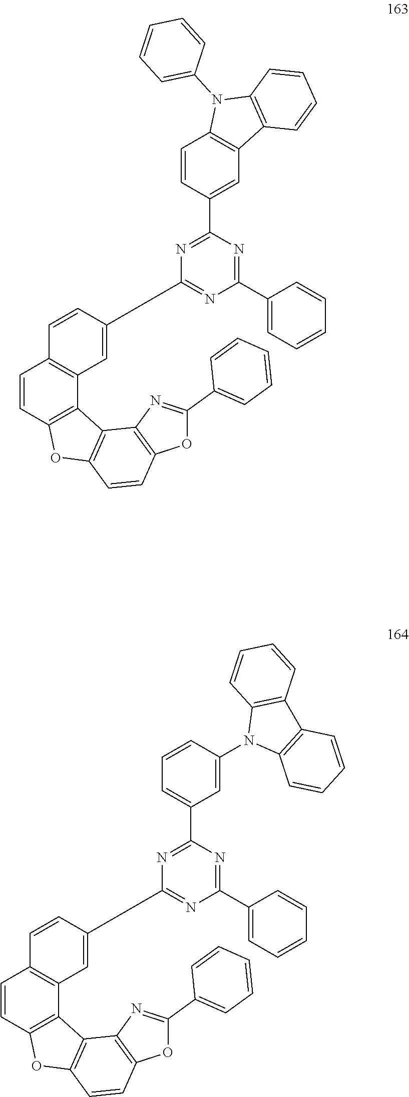 [SMILES: c1ccc(-c2nc(-c3ccc4ccc5oc6ccc7oc(-c8ccccc8)nc7c6c5c4c3)nc(-c3ccc4c(c3)c3ccccc3n4-c3ccccc3)n2)cc1 ; c1ccc(-c2nc(-c3cccc(-n4c5ccccc5c5ccccc54)c3)nc(-c3ccc4ccc5oc6ccc7oc(-c8ccccc8)nc7c6c5c4c3)n2)cc1]

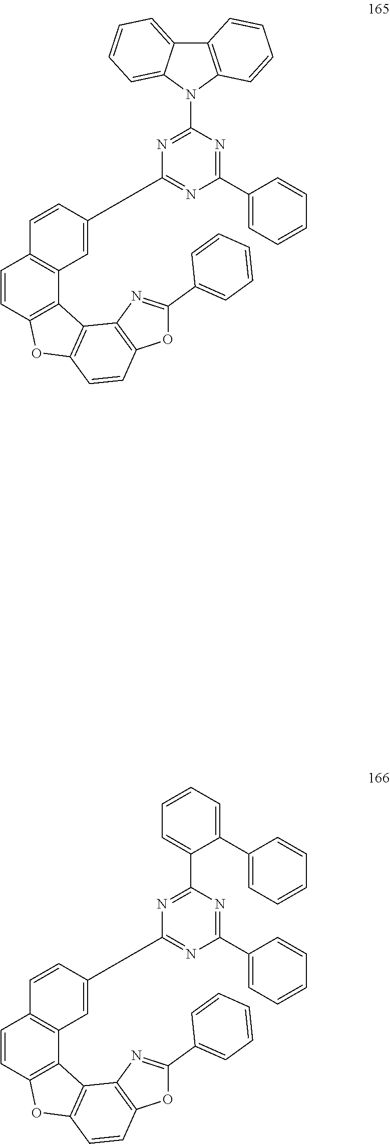 [SMILES: c1ccc(-c2nc(-c3ccc4ccc5oc6ccc7oc(-c8ccccc8)nc7c6c5c4c3)nc(-n3c4ccccc4c4ccccc43)n2)cc1 ; c1ccc(-c2nc(-c3ccc4ccc5oc6ccc7oc(-c8ccccc8)nc7c6c5c4c3)nc(-c3ccccc3-c3ccccc3)n2)cc1]

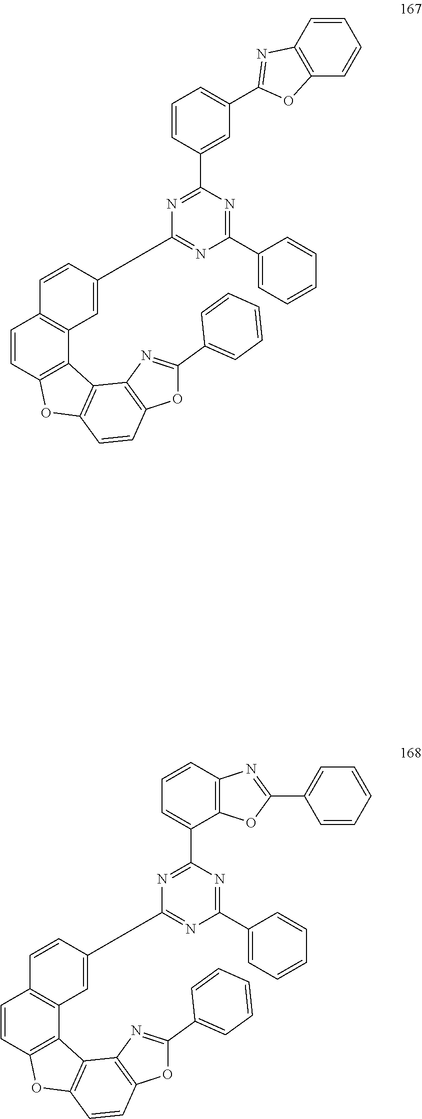 [SMILES: c1ccc(-c2nc(-c3cccc(-c4nc5ccccc5o4)c3)nc(-c3ccc4ccc5oc6ccc7oc(-c8ccccc8)nc7c6c5c4c3)n2)cc1 ; c1ccc(-c2nc(-c3ccc4ccc5oc6ccc7oc(-c8ccccc8)nc7c6c5c4c3)nc(-c3cccc4nc(-c5ccccc5)oc34)n2)cc1]

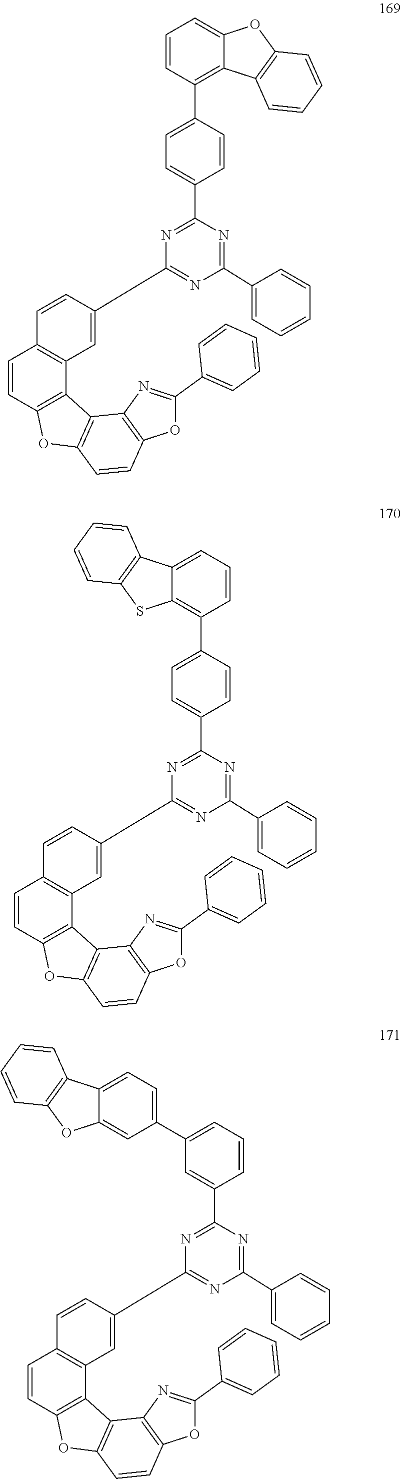 [SMILES: c1ccc(-c2nc(-c3ccc(-c4cccc5oc6ccccc6c45)cc3)nc(-c3ccc4ccc5oc6ccc7oc(-c8ccccc8)nc7c6c5c4c3)n2)cc1 ; c1ccc(-c2nc(-c3ccc(-c4cccc5c4sc4ccccc45)cc3)nc(-c3ccc4ccc5oc6ccc7oc(-c8ccccc8)nc7c6c5c4c3)n2)cc1 ; c1ccc(-c2nc(-c3cccc(-c4ccc5c(c4)oc4ccccc45)c3)nc(-c3ccc4ccc5oc6ccc7oc(-c8ccccc8)nc7c6c5c4c3)n2)cc1]

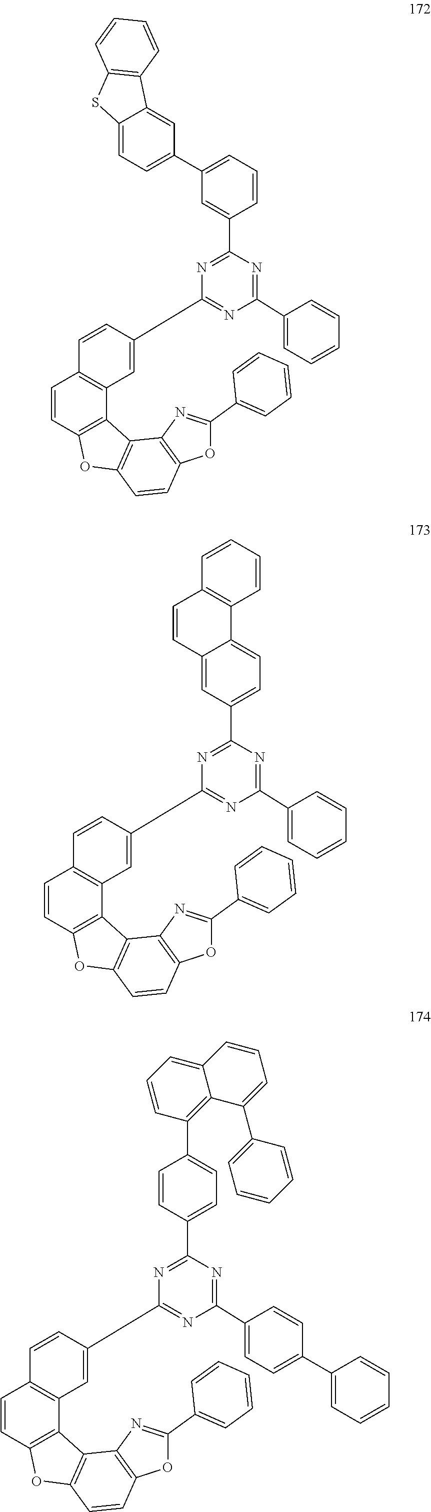 [SMILES: c1ccc(-c2nc(-c3cccc(-c4ccc5sc6ccccc6c5c4)c3)nc(-c3ccc4ccc5oc6ccc7oc(-c8ccccc8)nc7c6c5c4c3)n2)cc1 ; c1ccc(-c2nc(-c3ccc4c(ccc5ccccc54)c3)nc(-c3ccc4ccc5oc6ccc7oc(-c8ccccc8)nc7c6c5c4c3)n2)cc1 ; c1ccc(-c2ccc(-c3nc(-c4ccc(-c5cccc6cccc(-c7ccccc7)c56)cc4)nc(-c4ccc5ccc6oc7ccc8oc(-c9ccccc9)nc8c7c6c5c4)n3)cc2)cc1]

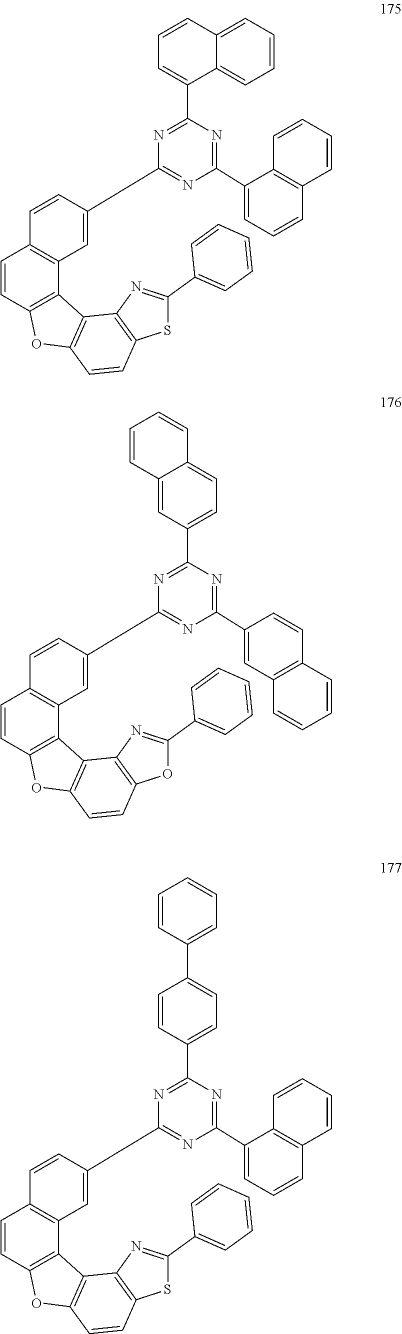 [SMILES: c1ccc(-c2nc3c(ccc4oc5ccc6ccc(-c7nc(-c8cccc9ccccc89)nc(-c8cccc9ccccc89)n7)cc6c5c43)s2)cc1 ; c1ccc(-c2nc3c(ccc4oc5ccc6ccc(-c7nc(-c8ccc9ccccc9c8)nc(-c8ccc9ccccc9c8)n7)cc6c5c43)o2)cc1 ; c1ccc(-c2ccc(-c3nc(-c4ccc5ccc6oc7ccc8sc(-c9ccccc9)nc8c7c6c5c4)nc(-c4cccc5ccccc45)n3)cc2)cc1]

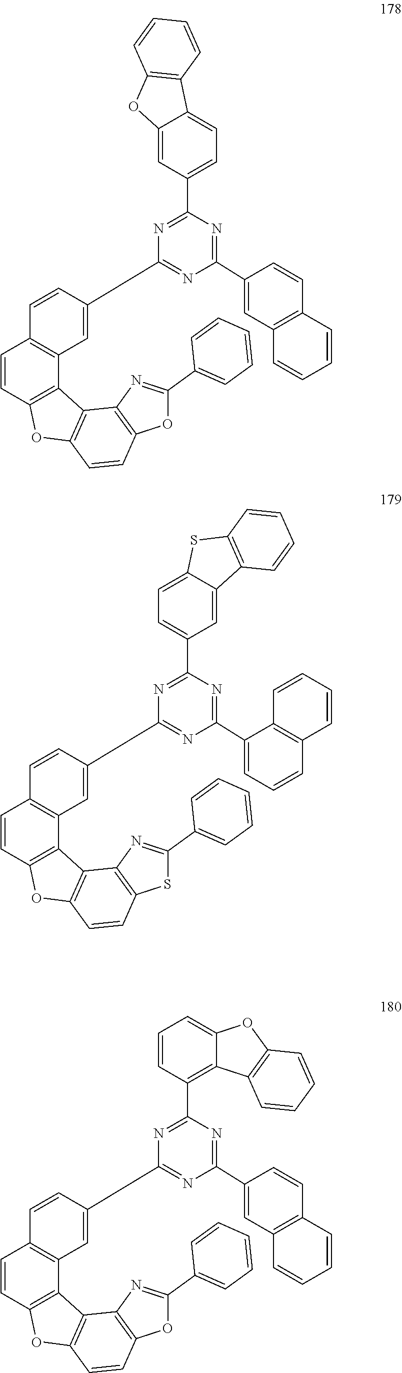 [SMILES: c1ccc(-c2nc3c(ccc4oc5ccc6ccc(-c7nc(-c8ccc9ccccc9c8)nc(-c8ccc9c(c8)oc8ccccc89)n7)cc6c5c43)o2)cc1 ; c1ccc(-c2nc3c(ccc4oc5ccc6ccc(-c7nc(-c8ccc9sc%10ccccc%10c9c8)nc(-c8cccc9ccccc89)n7)cc6c5c43)s2)cc1 ; c1ccc(-c2nc3c(ccc4oc5ccc6ccc(-c7nc(-c8ccc9ccccc9c8)nc(-c8cccc9oc%10ccccc%10c89)n7)cc6c5c43)o2)cc1]

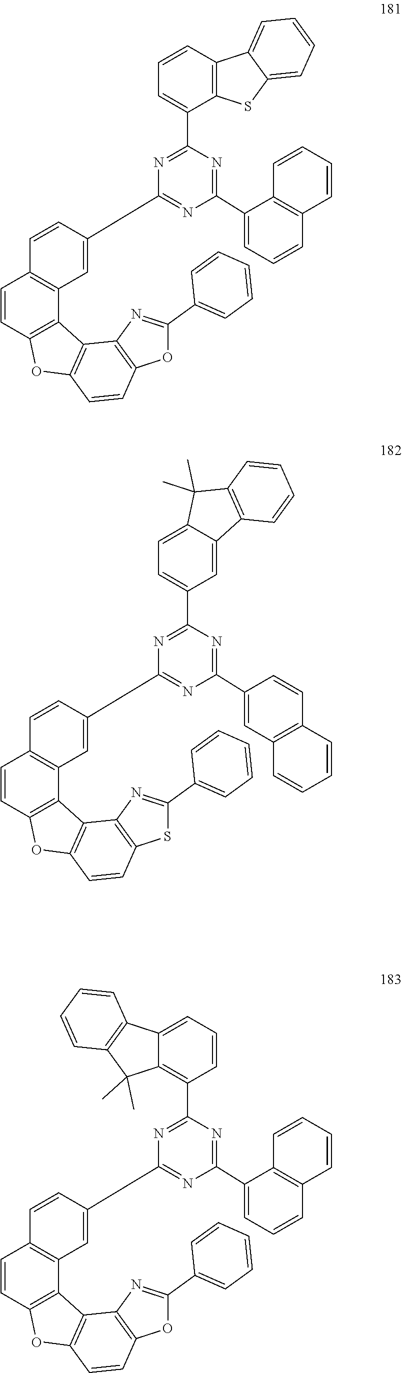 [SMILES: c1ccc(-c2nc3c(ccc4oc5ccc6ccc(-c7nc(-c8cccc9ccccc89)nc(-c8cccc9c8sc8ccccc89)n7)cc6c5c43)o2)cc1 ; CC1(C)c2ccccc2-c2cc(-c3nc(-c4ccc5ccccc5c4)nc(-c4ccc5ccc6oc7ccc8sc(-c9ccccc9)nc8c7c6c5c4)n3)ccc21 ; CC1(C)c2ccccc2-c2cccc(-c3nc(-c4ccc5ccc6oc7ccc8oc(-c9ccccc9)nc8c7c6c5c4)nc(-c4cccc5ccccc45)n3)c21]

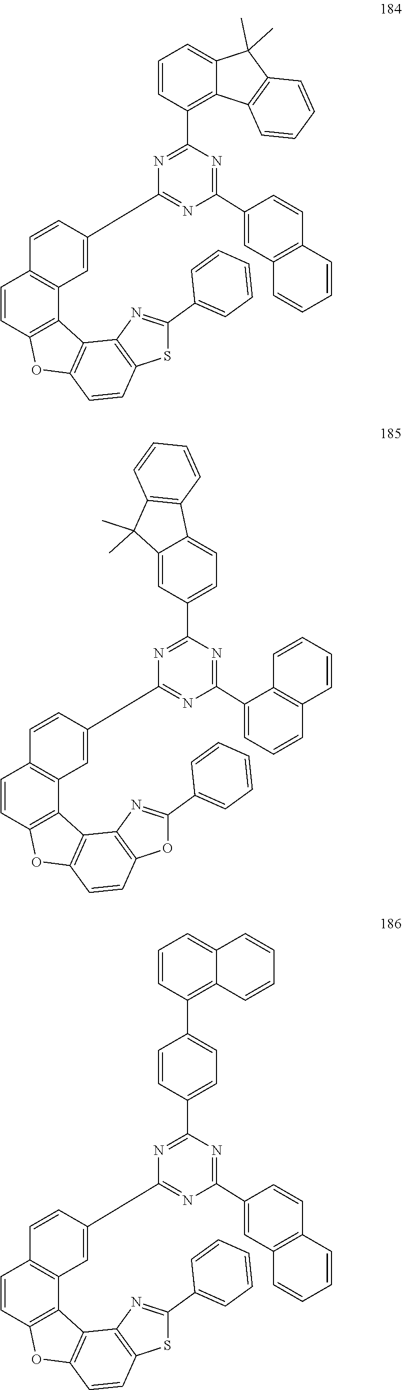 [SMILES: CC1(C)c2ccccc2-c2c(-c3nc(-c4ccc5ccccc5c4)nc(-c4ccc5ccc6oc7ccc8sc(-c9ccccc9)nc8c7c6c5c4)n3)cccc21 ; CC1(C)c2ccccc2-c2ccc(-c3nc(-c4ccc5ccc6oc7ccc8oc(-c9ccccc9)nc8c7c6c5c4)nc(-c4cccc5ccccc45)n3)cc21 ; c1ccc(-c2nc3c(ccc4oc5ccc6ccc(-c7nc(-c8ccc(-c9cccc%10ccccc9%10)cc8)nc(-c8ccc9ccccc9c8)n7)cc6c5c43)s2)cc1]

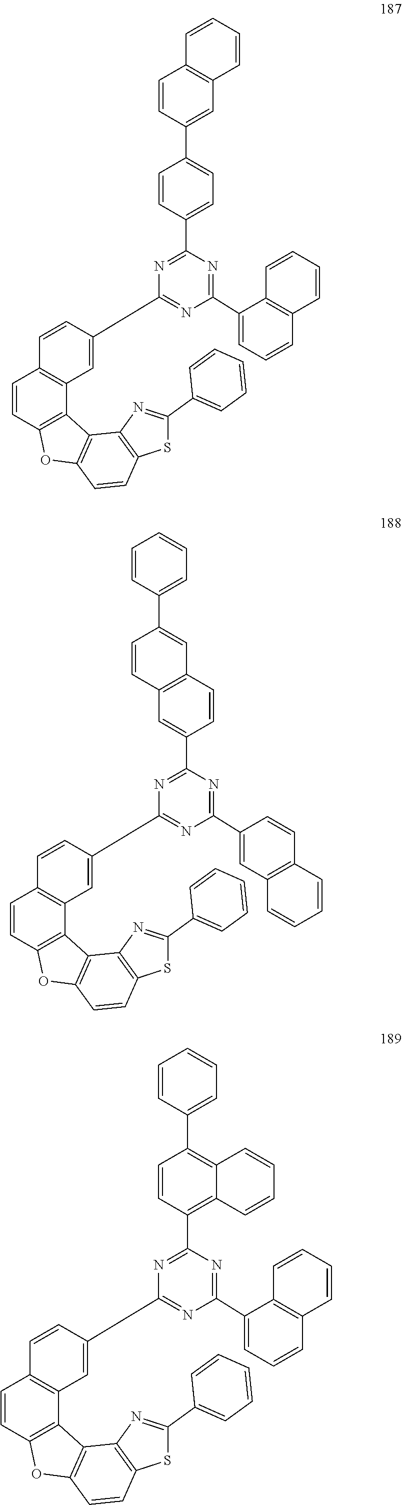 [SMILES: c1ccc(-c2nc3c(ccc4oc5ccc6ccc(-c7nc(-c8ccc(-c9ccc%10ccccc%10c9)cc8)nc(-c8cccc9ccccc89)n7)cc6c5c43)s2)cc1 ; c1ccc(-c2ccc3cc(-c4nc(-c5ccc6ccccc6c5)nc(-c5ccc6ccc7oc8ccc9sc(-c%10ccccc%10)nc9c8c7c6c5)n4)ccc3c2)cc1 ; c1ccc(-c2nc3c(ccc4oc5ccc6ccc(-c7nc(-c8cccc9ccccc89)nc(-c8ccc(-c9ccccc9)c9ccccc89)n7)cc6c5c43)s2)cc1]

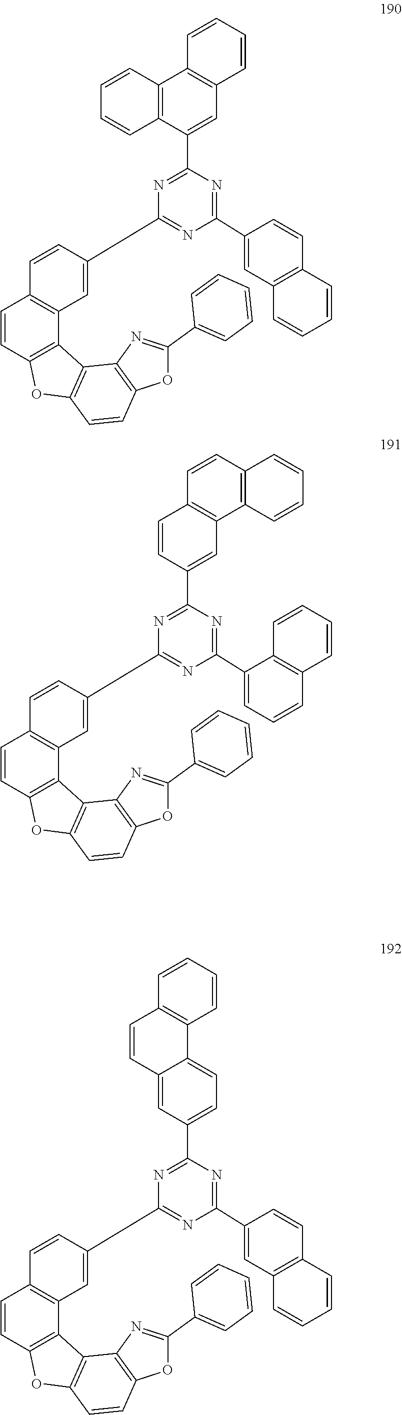 [SMILES: c1ccc(-c2nc3c(ccc4oc5ccc6ccc(-c7nc(-c8ccc9ccccc9c8)nc(-c8cc9ccccc9c9ccccc89)n7)cc6c5c43)o2)cc1 ; c1ccc(-c2nc3c(ccc4oc5ccc6ccc(-c7nc(-c8ccc9ccc%10ccccc%10c9c8)nc(-c8cccc9ccccc89)n7)cc6c5c43)o2)cc1 ; c1ccc(-c2nc3c(ccc4oc5ccc6ccc(-c7nc(-c8ccc9ccccc9c8)nc(-c8ccc9c(ccc%10ccccc%109)c8)n7)cc6c5c43)o2)cc1]

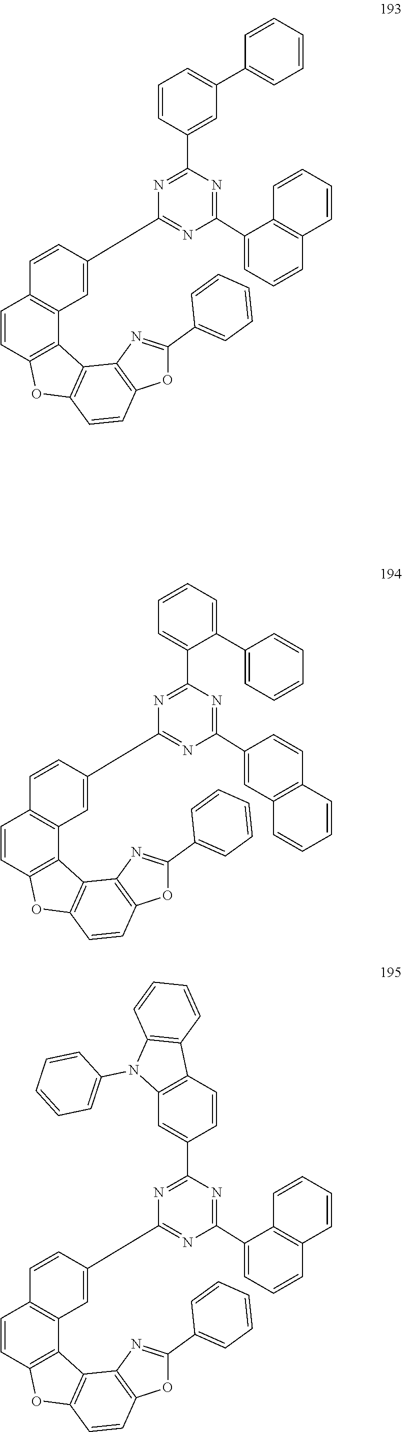 [SMILES: c1ccc(-c2cccc(-c3nc(-c4ccc5ccc6oc7ccc8oc(-c9ccccc9)nc8c7c6c5c4)nc(-c4cccc5ccccc45)n3)c2)cc1 ; c1ccc(-c2nc3c(ccc4oc5ccc6ccc(-c7nc(-c8ccc9ccccc9c8)nc(-c8ccccc8-c8ccccc8)n7)cc6c5c43)o2)cc1 ; c1ccc(-c2nc3c(ccc4oc5ccc6ccc(-c7nc(-c8ccc9c%10ccccc%10n(-c%10ccccc%10)c9c8)nc(-c8cccc9ccccc89)n7)cc6c5c43)o2)cc1]

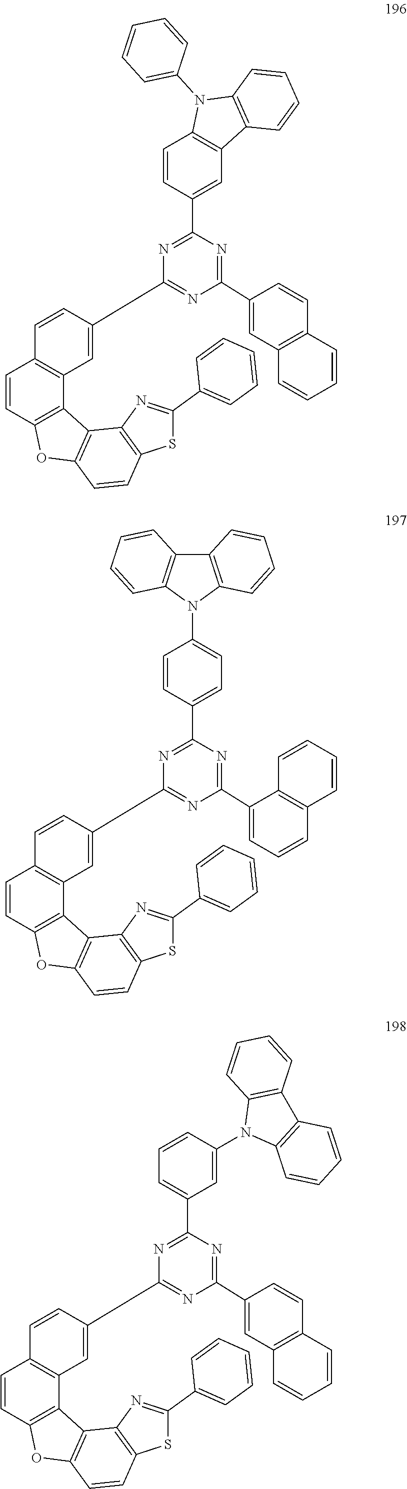 [SMILES: c1ccc(-c2nc3c(ccc4oc5ccc6ccc(-c7nc(-c8ccc9ccccc9c8)nc(-c8ccc9c(c8)c8ccccc8n9-c8ccccc8)n7)cc6c5c43)s2)cc1 ; c1ccc(-c2nc3c(ccc4oc5ccc6ccc(-c7nc(-c8ccc(-n9c%10ccccc%10c%10ccccc%109)cc8)nc(-c8cccc9ccccc89)n7)cc6c5c43)s2)cc1 ; c1ccc(-c2nc3c(ccc4oc5ccc6ccc(-c7nc(-c8cccc(-n9c%10ccccc%10c%10ccccc%109)c8)nc(-c8ccc9ccccc9c8)n7)cc6c5c43)s2)cc1]

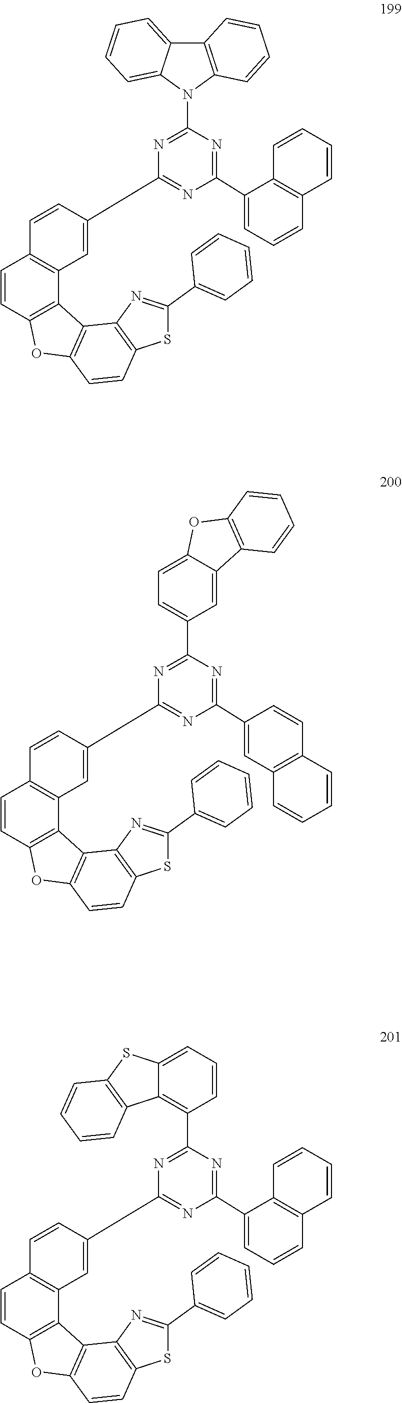 [SMILES: c1ccc(-c2nc3c(ccc4oc5ccc6ccc(-c7nc(-c8cccc9ccccc89)nc(-n8c9ccccc9c9ccccc98)n7)cc6c5c43)s2)cc1 ; c1ccc(-c2nc3c(ccc4oc5ccc6ccc(-c7nc(-c8ccc9ccccc9c8)nc(-c8ccc9oc%10ccccc%10c9c8)n7)cc6c5c43)s2)cc1 ; c1ccc(-c2nc3c(ccc4oc5ccc6ccc(-c7nc(-c8cccc9ccccc89)nc(-c8cccc9sc%10ccccc%10c89)n7)cc6c5c43)s2)cc1]

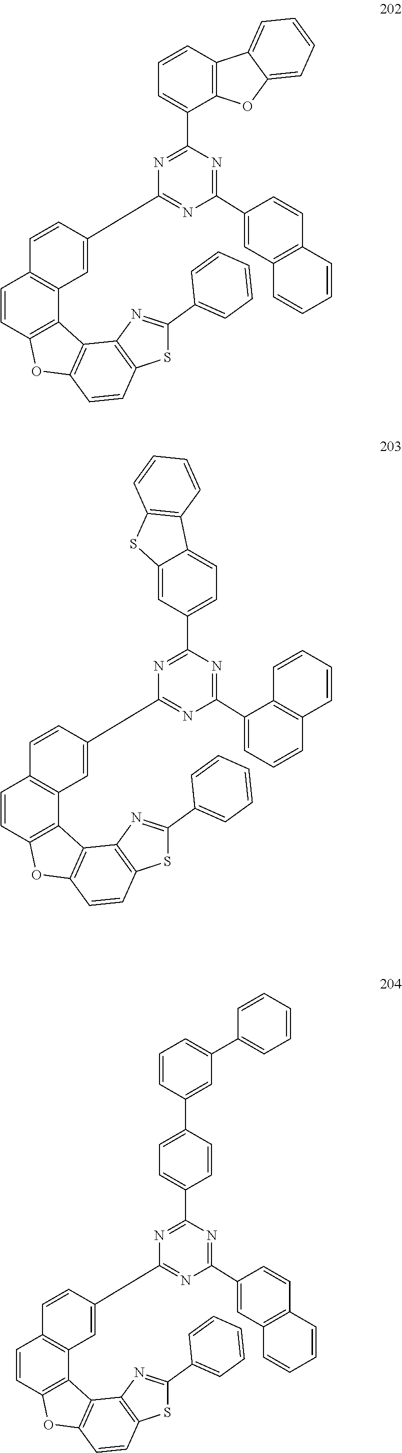 [SMILES: c1ccc(-c2nc3c(ccc4oc5ccc6ccc(-c7nc(-c8ccc9ccccc9c8)nc(-c8cccc9c8oc8ccccc89)n7)cc6c5c43)s2)cc1 ; c1ccc(-c2nc3c(ccc4oc5ccc6ccc(-c7nc(-c8ccc9c(c8)sc8ccccc89)nc(-c8cccc9ccccc89)n7)cc6c5c43)s2)cc1 ; c1ccc(-c2cccc(-c3ccc(-c4nc(-c5ccc6ccccc6c5)nc(-c5ccc6ccc7oc8ccc9sc(-c%10ccccc%10)nc9c8c7c6c5)n4)cc3)c2)cc1]

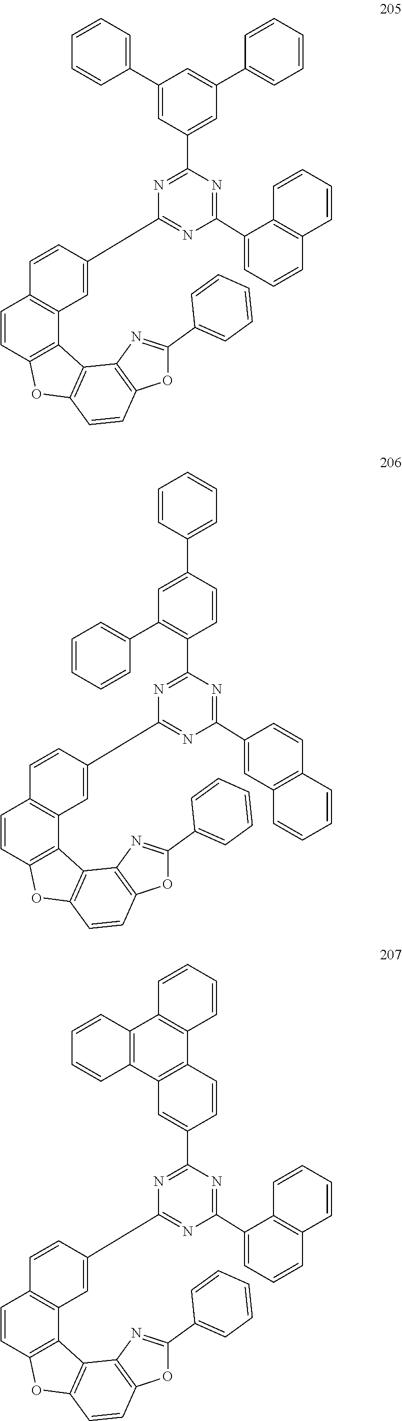 [SMILES: c1ccc(-c2cc(-c3ccccc3)cc(-c3nc(-c4ccc5ccc6oc7ccc8oc(-c9ccccc9)nc8c7c6c5c4)nc(-c4cccc5ccccc45)n3)c2)cc1 ; c1ccc(-c2ccc(-c3nc(-c4ccc5ccccc5c4)nc(-c4ccc5ccc6oc7ccc8oc(-c9ccccc9)nc8c7c6c5c4)n3)c(-c3ccccc3)c2)cc1 ; c1ccc(-c2nc3c(ccc4oc5ccc6ccc(-c7nc(-c8ccc9c%10ccccc%10c%10ccccc%10c9c8)nc(-c8cccc9ccccc89)n7)cc6c5c43)o2)cc1]

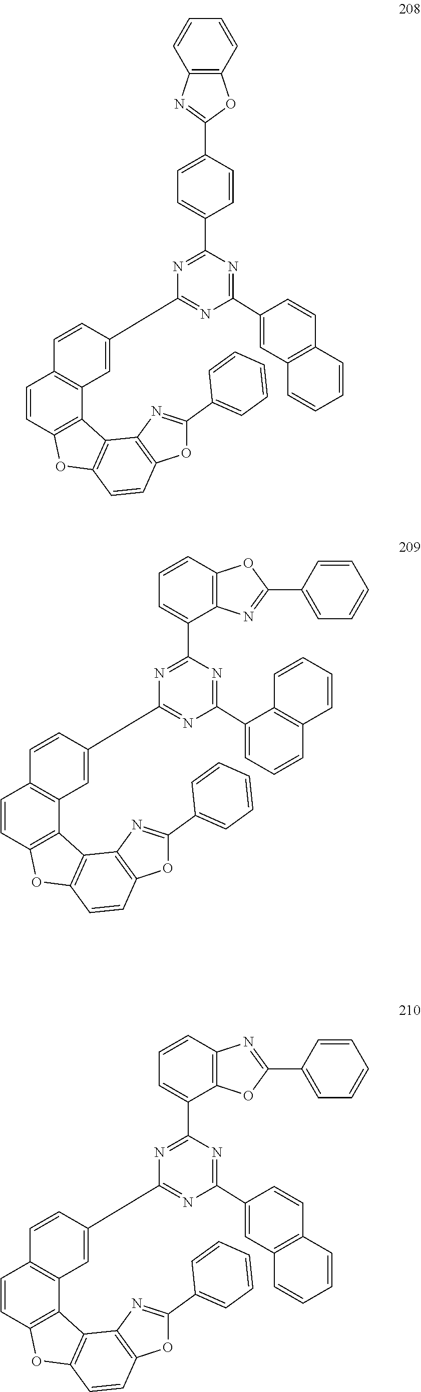 [SMILES: c1ccc(-c2nc3c(ccc4oc5ccc6ccc(-c7nc(-c8ccc(-c9nc%10ccccc%10o9)cc8)nc(-c8ccc9ccccc9c8)n7)cc6c5c43)o2)cc1 ; c1ccc(-c2nc3c(-c4nc(-c5ccc6ccc7oc8ccc9oc(-c%10ccccc%10)nc9c8c7c6c5)nc(-c5cccc6ccccc56)n4)cccc3o2)cc1 ; c1ccc(-c2nc3c(ccc4oc5ccc6ccc(-c7nc(-c8ccc9ccccc9c8)nc(-c8cccc9nc(-c%10ccccc%10)oc89)n7)cc6c5c43)o2)cc1]

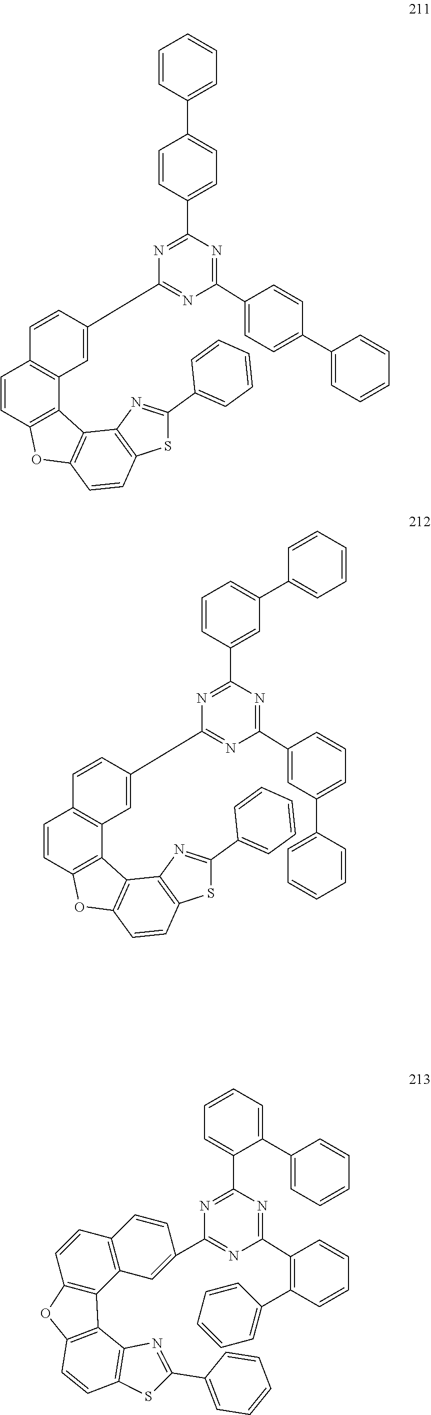 [SMILES: c1ccc(-c2ccc(-c3nc(-c4ccc(-c5ccccc5)cc4)nc(-c4ccc5ccc6oc7ccc8sc(-c9ccccc9)nc8c7c6c5c4)n3)cc2)cc1 ; c1ccc(-c2cccc(-c3nc(-c4cccc(-c5ccccc5)c4)nc(-c4ccc5ccc6oc7ccc8sc(-c9ccccc9)nc8c7c6c5c4)n3)c2)cc1 ; c1ccc(-c2nc3c(ccc4oc5ccc6ccc(-c7nc(-c8ccccc8-c8ccccc8)nc(-c8ccccc8-c8ccccc8)n7)cc6c5c43)s2)cc1]

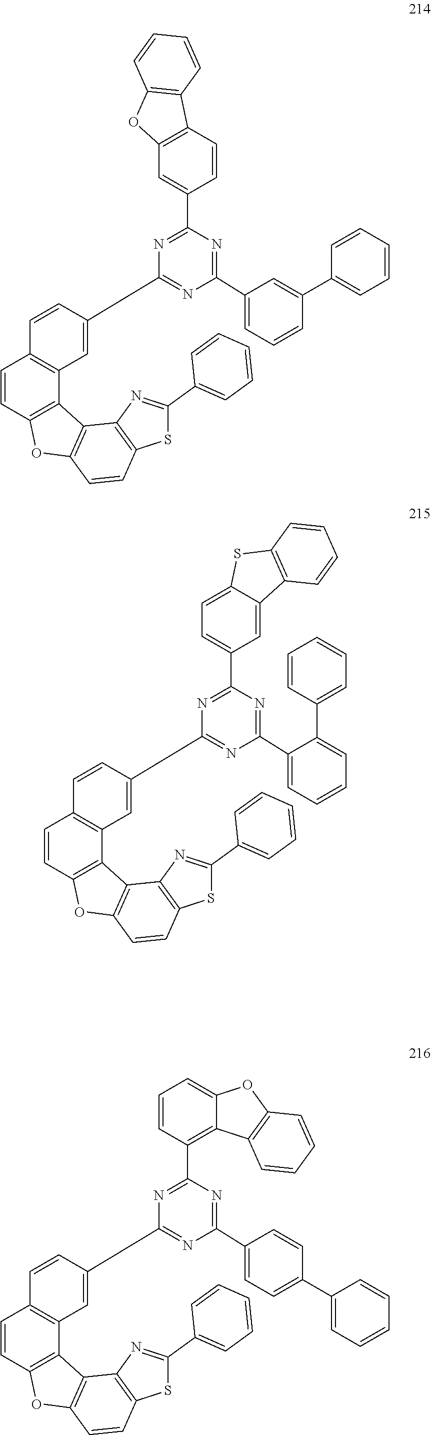 [SMILES: c1ccc(-c2cccc(-c3nc(-c4ccc5c(c4)oc4ccccc45)nc(-c4ccc5ccc6oc7ccc8sc(-c9ccccc9)nc8c7c6c5c4)n3)c2)cc1 ; c1ccc(-c2nc3c(ccc4oc5ccc6ccc(-c7nc(-c8ccc9sc%10ccccc%10c9c8)nc(-c8ccccc8-c8ccccc8)n7)cc6c5c43)s2)cc1 ; c1ccc(-c2ccc(-c3nc(-c4ccc5ccc6oc7ccc8sc(-c9ccccc9)nc8c7c6c5c4)nc(-c4cccc5oc6ccccc6c45)n3)cc2)cc1]

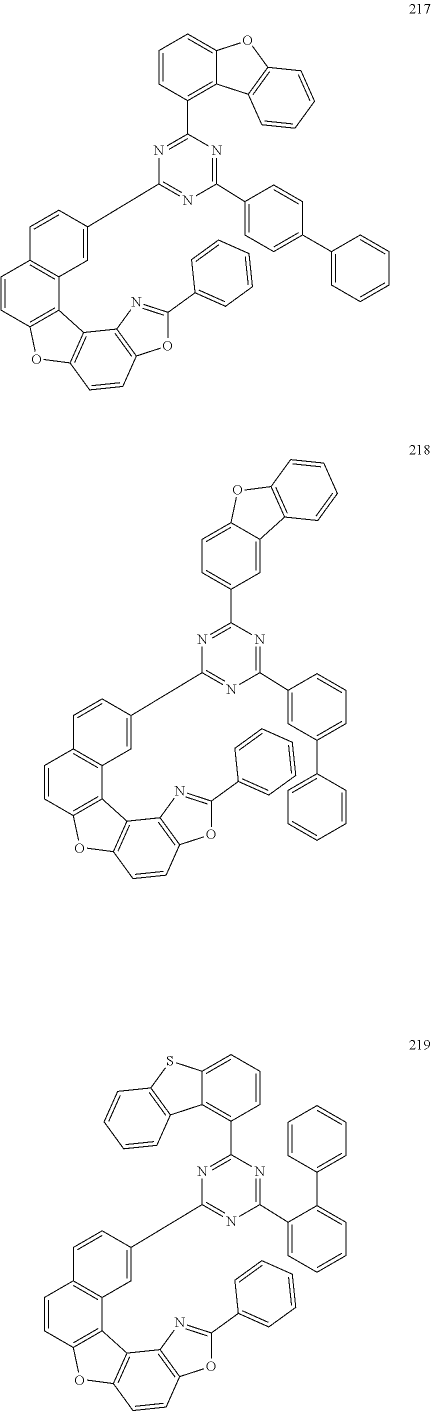 [SMILES: c1ccc(-c2ccc(-c3nc(-c4ccc5ccc6oc7ccc8oc(-c9ccccc9)nc8c7c6c5c4)nc(-c4cccc5oc6ccccc6c45)n3)cc2)cc1 ; c1ccc(-c2cccc(-c3nc(-c4ccc5oc6ccccc6c5c4)nc(-c4ccc5ccc6oc7ccc8oc(-c9ccccc9)nc8c7c6c5c4)n3)c2)cc1 ; c1ccc(-c2nc3c(ccc4oc5ccc6ccc(-c7nc(-c8ccccc8-c8ccccc8)nc(-c8cccc9sc%10ccccc%10c89)n7)cc6c5c43)o2)cc1]

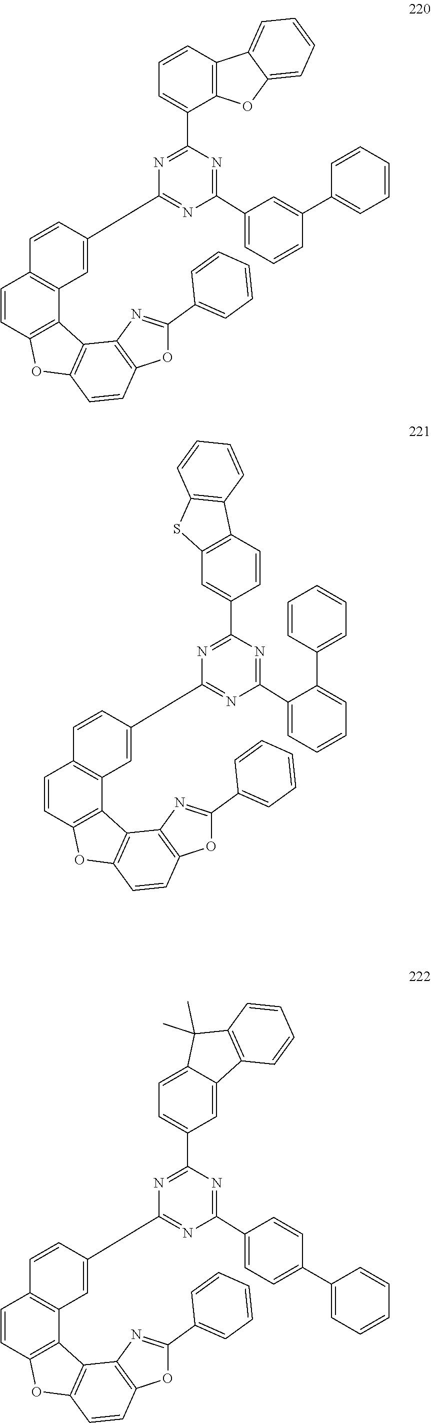 [SMILES: c1ccc(-c2cccc(-c3nc(-c4ccc5ccc6oc7ccc8oc(-c9ccccc9)nc8c7c6c5c4)nc(-c4cccc5c4oc4ccccc45)n3)c2)cc1 ; c1ccc(-c2nc3c(ccc4oc5ccc6ccc(-c7nc(-c8ccc9c(c8)sc8ccccc89)nc(-c8ccccc8-c8ccccc8)n7)cc6c5c43)o2)cc1 ; CC1(C)c2ccccc2-c2cc(-c3nc(-c4ccc(-c5ccccc5)cc4)nc(-c4ccc5ccc6oc7ccc8oc(-c9ccccc9)nc8c7c6c5c4)n3)ccc21]

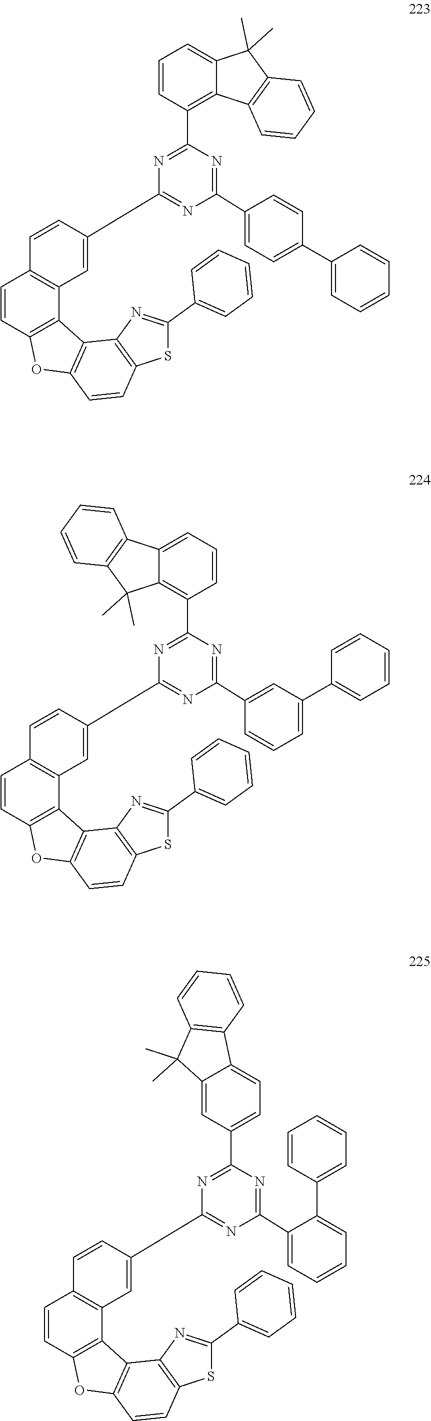 [SMILES: CC1(C)c2ccccc2-c2c(-c3nc(-c4ccc(-c5ccccc5)cc4)nc(-c4ccc5ccc6oc7ccc8sc(-c9ccccc9)nc8c7c6c5c4)n3)cccc21 ; CC1(C)c2ccccc2-c2cccc(-c3nc(-c4cccc(-c5ccccc5)c4)nc(-c4ccc5ccc6oc7ccc8sc(-c9ccccc9)nc8c7c6c5c4)n3)c21 ; CC1(C)c2ccccc2-c2ccc(-c3nc(-c4ccc5ccc6oc7ccc8sc(-c9ccccc9)nc8c7c6c5c4)nc(-c4ccccc4-c4ccccc4)n3)cc21]

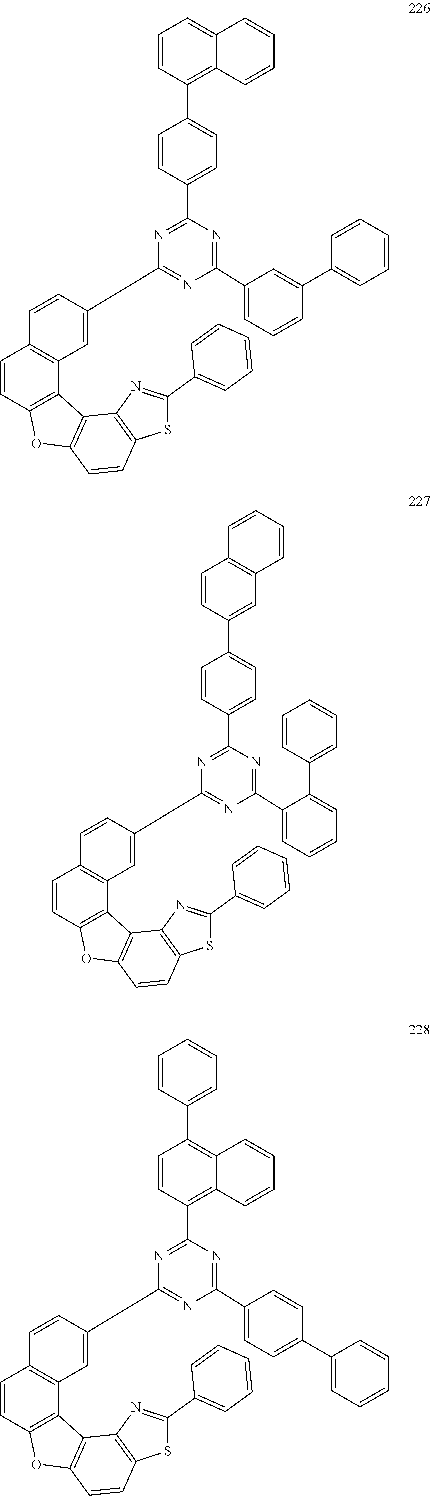 [SMILES: c1ccc(-c2cccc(-c3nc(-c4ccc(-c5cccc6ccccc56)cc4)nc(-c4ccc5ccc6oc7ccc8sc(-c9ccccc9)nc8c7c6c5c4)n3)c2)cc1 ; c1ccc(-c2nc3c(ccc4oc5ccc6ccc(-c7nc(-c8ccc(-c9ccc%10ccccc%10c9)cc8)nc(-c8ccccc8-c8ccccc8)n7)cc6c5c43)s2)cc1 ; c1ccc(-c2ccc(-c3nc(-c4ccc5ccc6oc7ccc8sc(-c9ccccc9)nc8c7c6c5c4)nc(-c4ccc(-c5ccccc5)c5ccccc45)n3)cc2)cc1]

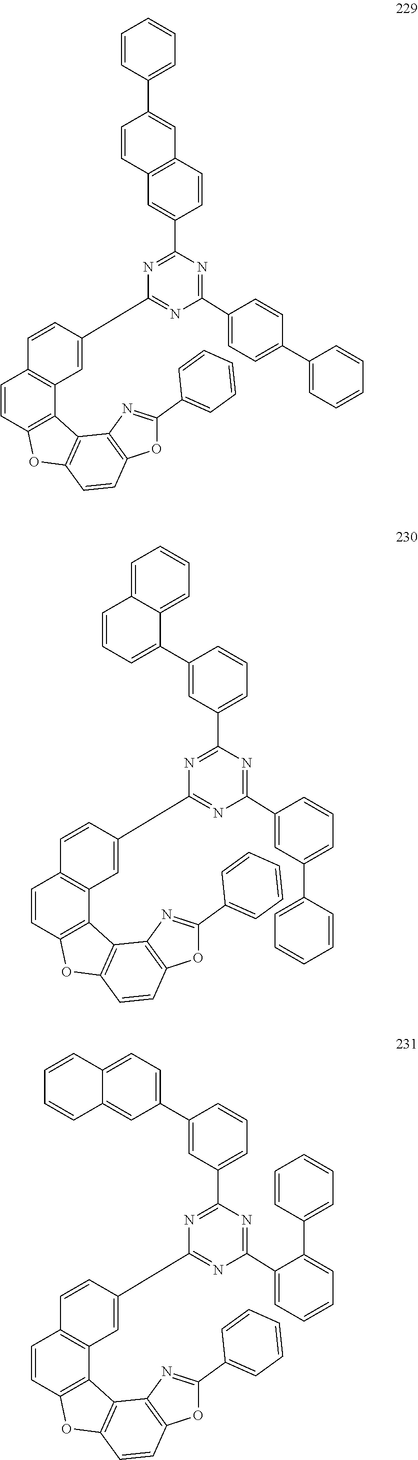 [SMILES: c1ccc(-c2ccc(-c3nc(-c4ccc5cc(-c6ccccc6)ccc5c4)nc(-c4ccc5ccc6oc7ccc8oc(-c9ccccc9)nc8c7c6c5c4)n3)cc2)cc1 ; c1ccc(-c2cccc(-c3nc(-c4cccc(-c5cccc6ccccc56)c4)nc(-c4ccc5ccc6oc7ccc8oc(-c9ccccc9)nc8c7c6c5c4)n3)c2)cc1 ; c1ccc(-c2nc3c(ccc4oc5ccc6ccc(-c7nc(-c8cccc(-c9ccc%10ccccc%10c9)c8)nc(-c8ccccc8-c8ccccc8)n7)cc6c5c43)o2)cc1]

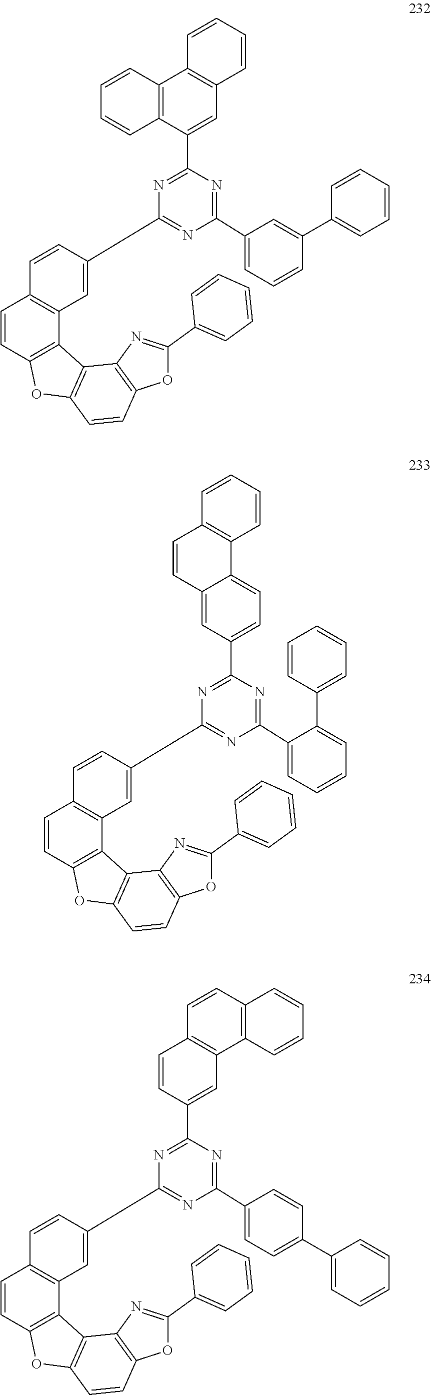 [SMILES: c1ccc(-c2cccc(-c3nc(-c4ccc5ccc6oc7ccc8oc(-c9ccccc9)nc8c7c6c5c4)nc(-c4cc5ccccc5c5ccccc45)n3)c2)cc1 ; c1ccc(-c2nc3c(ccc4oc5ccc6ccc(-c7nc(-c8ccc9c(ccc%10ccccc%109)c8)nc(-c8ccccc8-c8ccccc8)n7)cc6c5c43)o2)cc1 ; c1ccc(-c2ccc(-c3nc(-c4ccc5ccc6ccccc6c5c4)nc(-c4ccc5ccc6oc7ccc8oc(-c9ccccc9)nc8c7c6c5c4)n3)cc2)cc1]

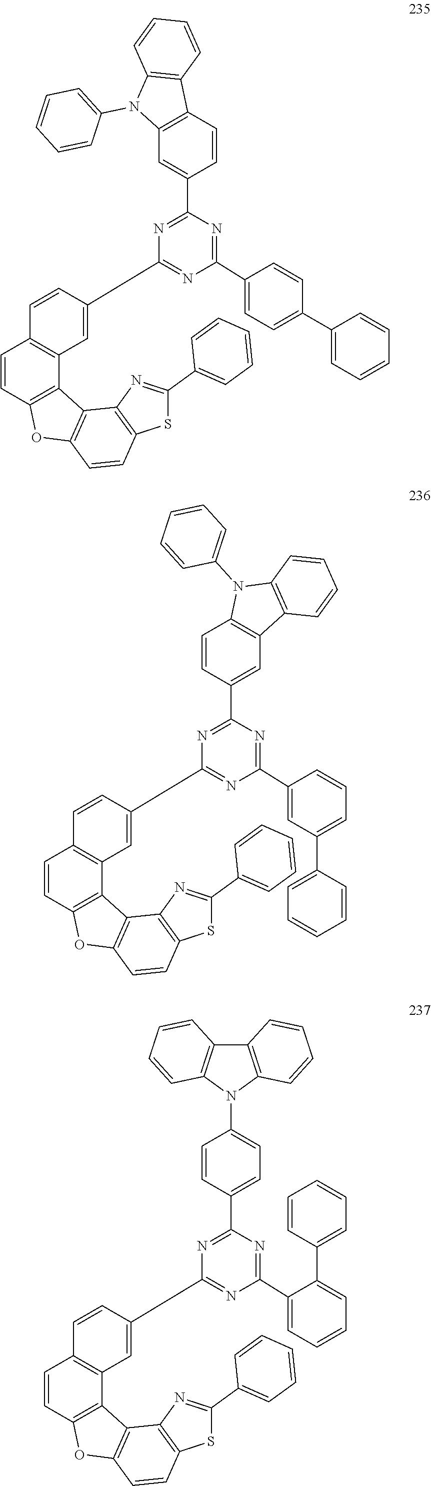 [SMILES: c1ccc(-c2ccc(-c3nc(-c4ccc5ccc6oc7ccc8sc(-c9ccccc9)nc8c7c6c5c4)nc(-c4ccc5c6ccccc6n(-c6ccccc6)c5c4)n3)cc2)cc1 ; c1ccc(-c2cccc(-c3nc(-c4ccc5ccc6oc7ccc8sc(-c9ccccc9)nc8c7c6c5c4)nc(-c4ccc5c(c4)c4ccccc4n5-c4ccccc4)n3)c2)cc1 ; c1ccc(-c2nc3c(ccc4oc5ccc6ccc(-c7nc(-c8ccc(-n9c%10ccccc%10c%10ccccc%109)cc8)nc(-c8ccccc8-c8ccccc8)n7)cc6c5c43)s2)cc1]

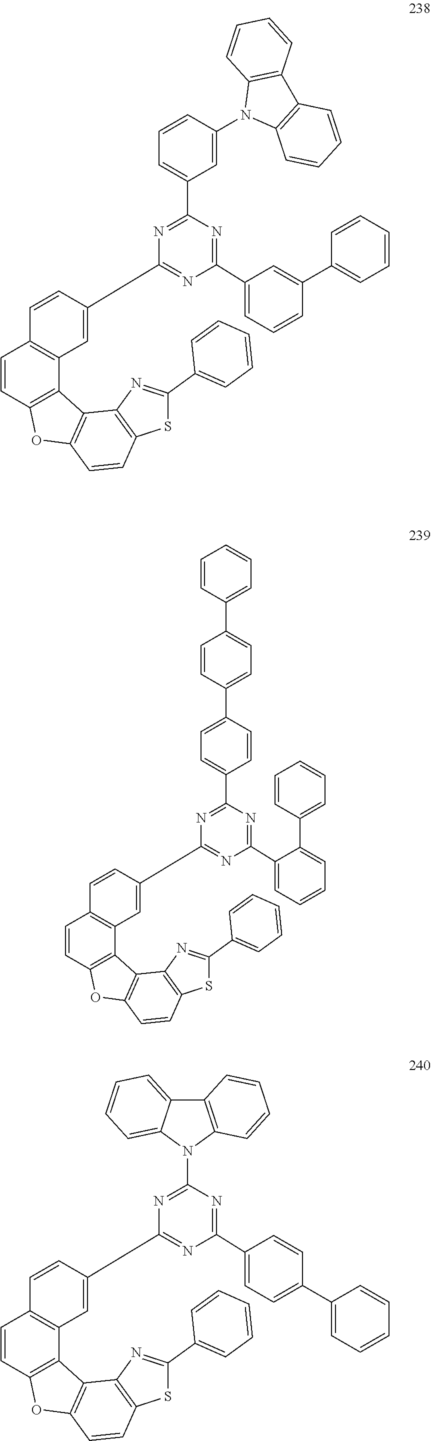 [SMILES: c1ccc(-c2cccc(-c3nc(-c4cccc(-n5c6ccccc6c6ccccc65)c4)nc(-c4ccc5ccc6oc7ccc8sc(-c9ccccc9)nc8c7c6c5c4)n3)c2)cc1 ; c1ccc(-c2ccc(-c3ccc(-c4nc(-c5ccc6ccc7oc8ccc9sc(-c%10ccccc%10)nc9c8c7c6c5)nc(-c5ccccc5-c5ccccc5)n4)cc3)cc2)cc1 ; c1ccc(-c2ccc(-c3nc(-c4ccc5ccc6oc7ccc8sc(-c9ccccc9)nc8c7c6c5c4)nc(-n4c5ccccc5c5ccccc54)n3)cc2)cc1]

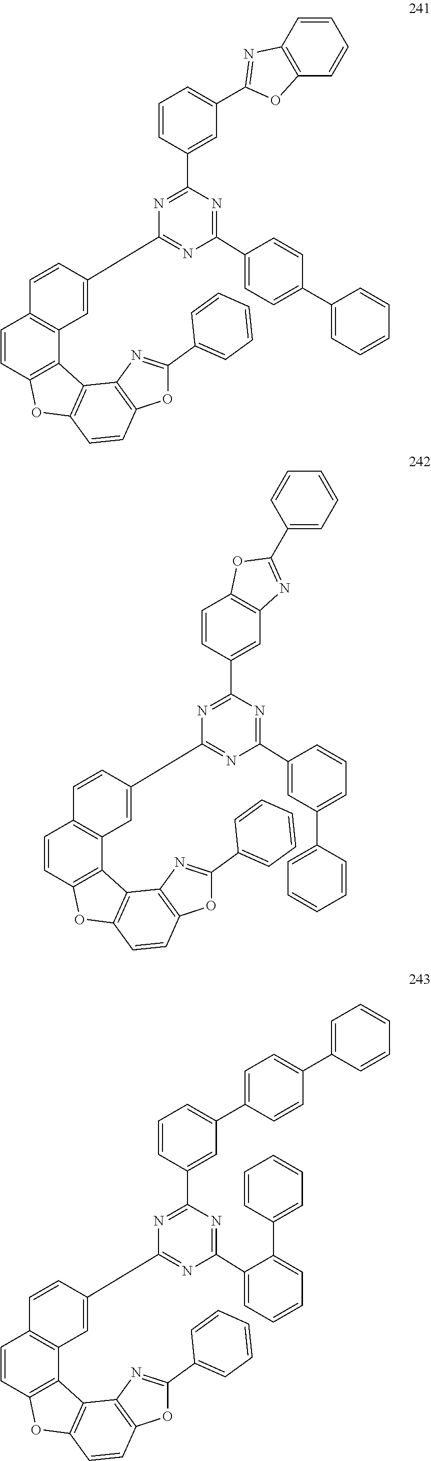 [SMILES: c1ccc(-c2ccc(-c3nc(-c4cccc(-c5nc6ccccc6o5)c4)nc(-c4ccc5ccc6oc7ccc8oc(-c9ccccc9)nc8c7c6c5c4)n3)cc2)cc1 ; c1ccc(-c2cccc(-c3nc(-c4ccc5oc(-c6ccccc6)nc5c4)nc(-c4ccc5ccc6oc7ccc8oc(-c9ccccc9)nc8c7c6c5c4)n3)c2)cc1 ; c1ccc(-c2ccc(-c3cccc(-c4nc(-c5ccc6ccc7oc8ccc9oc(-c%10ccccc%10)nc9c8c7c6c5)nc(-c5ccccc5-c5ccccc5)n4)c3)cc2)cc1]

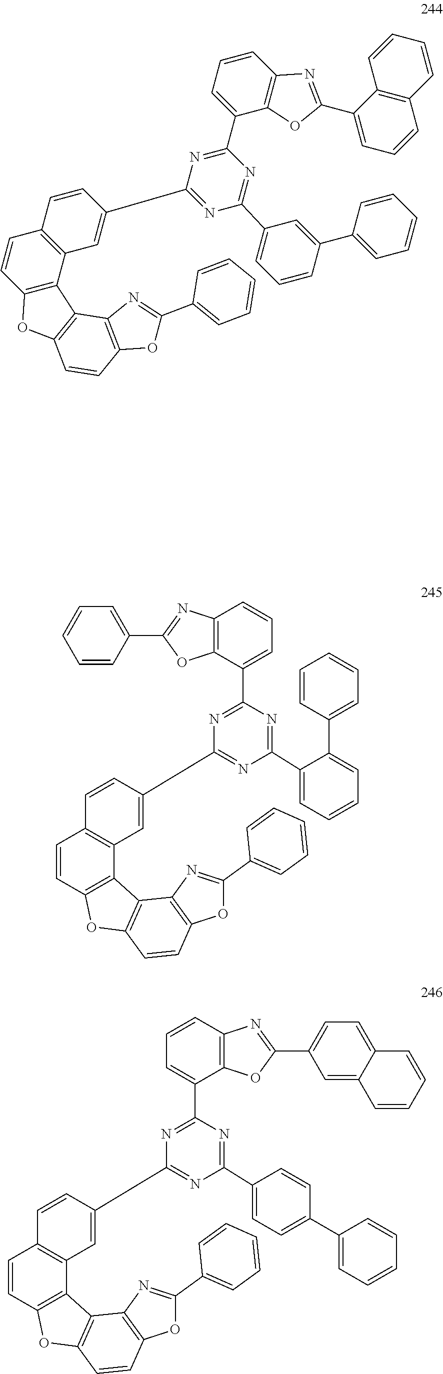 [SMILES: c1ccc(-c2cccc(-c3nc(-c4ccc5ccc6oc7ccc8oc(-c9ccccc9)nc8c7c6c5c4)nc(-c4cccc5nc(-c6cccc7ccccc67)oc45)n3)c2)cc1 ; c1ccc(-c2nc3c(ccc4oc5ccc6ccc(-c7nc(-c8ccccc8-c8ccccc8)nc(-c8cccc9nc(-c%10ccccc%10)oc89)n7)cc6c5c43)o2)cc1 ; c1ccc(-c2ccc(-c3nc(-c4ccc5ccc6oc7ccc8oc(-c9ccccc9)nc8c7c6c5c4)nc(-c4cccc5nc(-c6ccc7ccccc7c6)oc45)n3)cc2)cc1]

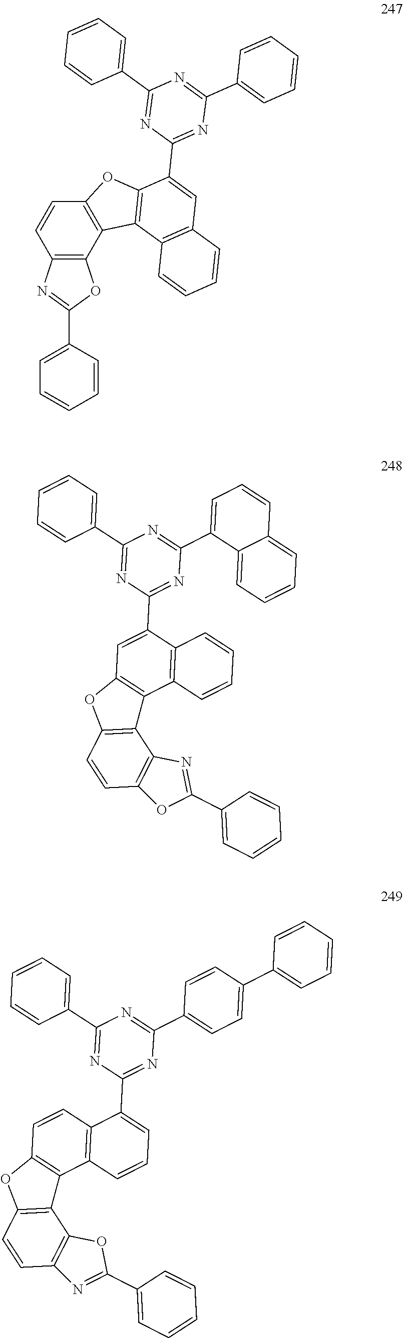 [SMILES: c1ccc(-c2nc(-c3ccccc3)nc(-c3cc4ccccc4c4c3oc3ccc5nc(-c6ccccc6)oc5c34)n2)cc1 ; c1ccc(-c2nc(-c3cccc4ccccc34)nc(-c3cc4oc5ccc6oc(-c7ccccc7)nc6c5c4c4ccccc34)n2)cc1 ; c1ccc(-c2ccc(-c3nc(-c4ccccc4)nc(-c4cccc5c4ccc4oc6ccc7nc(-c8ccccc8)oc7c6c45)n3)cc2)cc1]

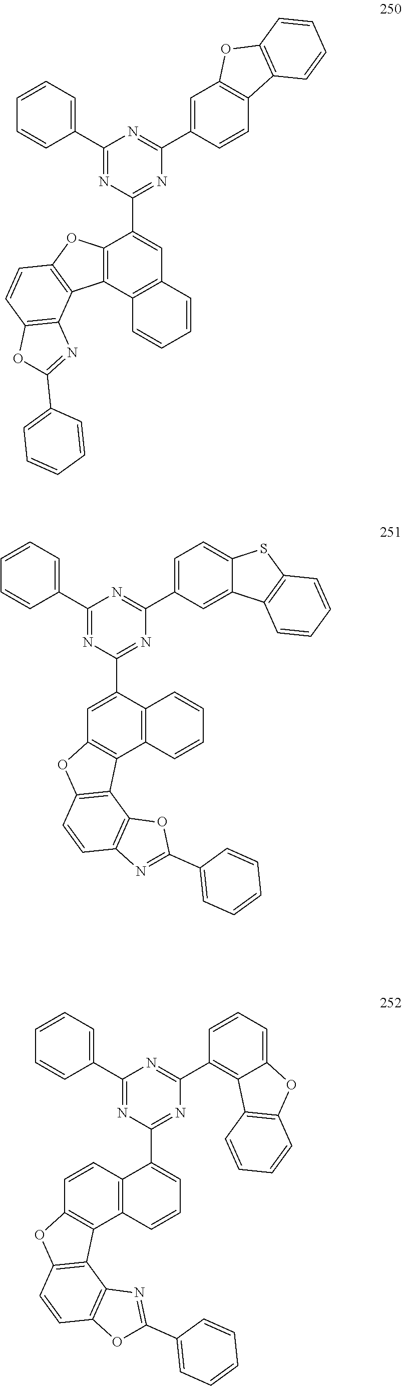 [SMILES: c1ccc(-c2nc(-c3ccc4c(c3)oc3ccccc34)nc(-c3cc4ccccc4c4c3oc3ccc5oc(-c6ccccc6)nc5c34)n2)cc1 ; c1ccc(-c2nc(-c3ccc4sc5ccccc5c4c3)nc(-c3cc4oc5ccc6nc(-c7ccccc7)oc6c5c4c4ccccc34)n2)cc1 ; c1ccc(-c2nc(-c3cccc4c3ccc3oc5ccc6oc(-c7ccccc7)nc6c5c34)nc(-c3cccc4oc5ccccc5c34)n2)cc1]

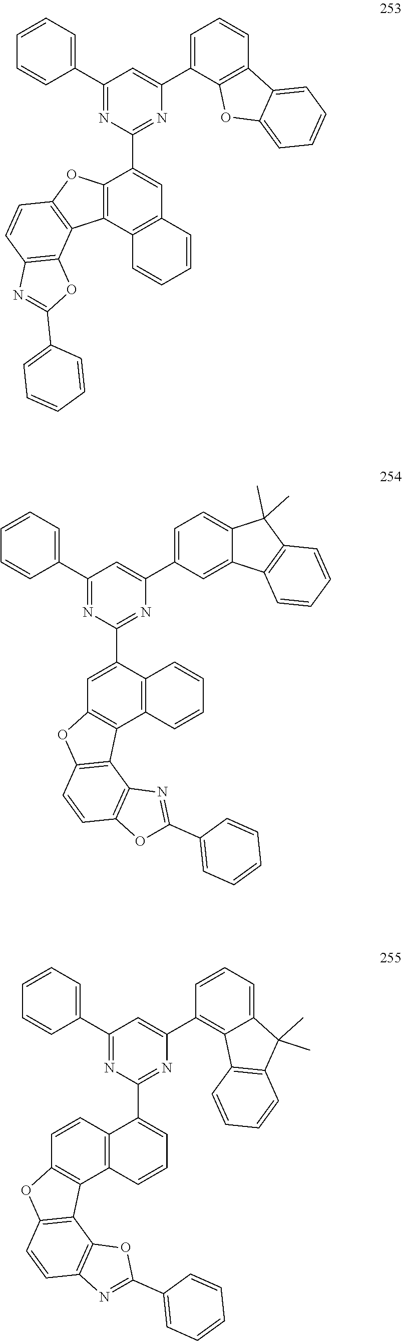 [SMILES: c1ccc(-c2cc(-c3cccc4c3oc3ccccc34)nc(-c3cc4ccccc4c4c3oc3ccc5nc(-c6ccccc6)oc5c34)n2)cc1 ; CC1(C)c2ccccc2-c2cc(-c3cc(-c4ccccc4)nc(-c4cc5oc6ccc7oc(-c8ccccc8)nc7c6c5c5ccccc45)n3)ccc21 ; CC1(C)c2ccccc2-c2c(-c3cc(-c4ccccc4)nc(-c4cccc5c4ccc4oc6ccc7nc(-c8ccccc8)oc7c6c45)n3)cccc21]

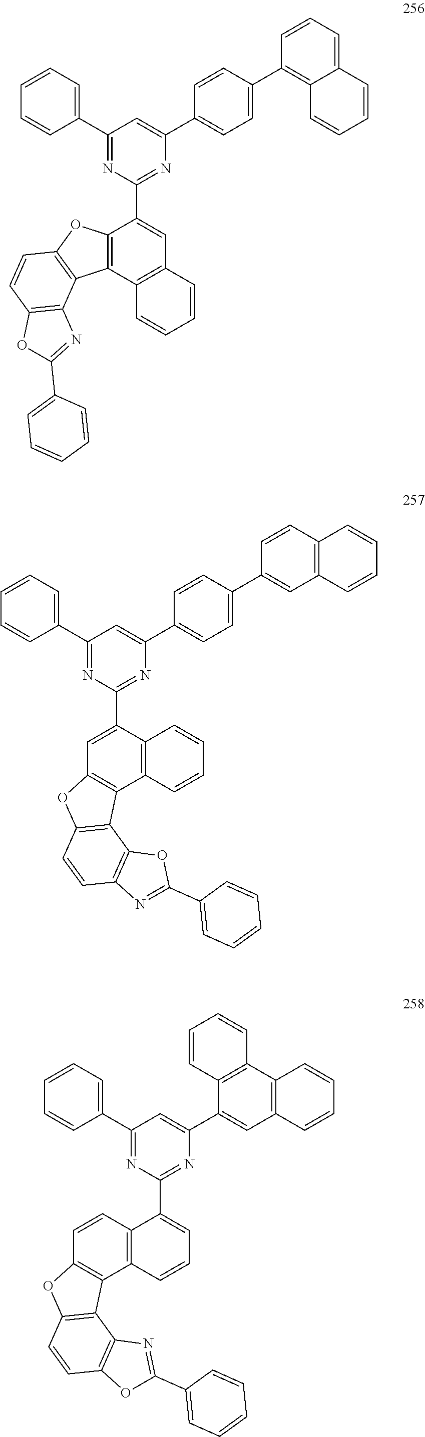 [SMILES: c1ccc(-c2cc(-c3ccc(-c4cccc5ccccc45)cc3)nc(-c3cc4ccccc4c4c3oc3ccc5oc(-c6ccccc6)nc5c34)n2)cc1 ; c1ccc(-c2cc(-c3ccc(-c4ccc5ccccc5c4)cc3)nc(-c3cc4oc5ccc6nc(-c7ccccc7)oc6c5c4c4ccccc34)n2)cc1 ; c1ccc(-c2cc(-c3cc4ccccc4c4ccccc34)nc(-c3cccc4c3ccc3oc5ccc6oc(-c7ccccc7)nc6c5c34)n2)cc1]

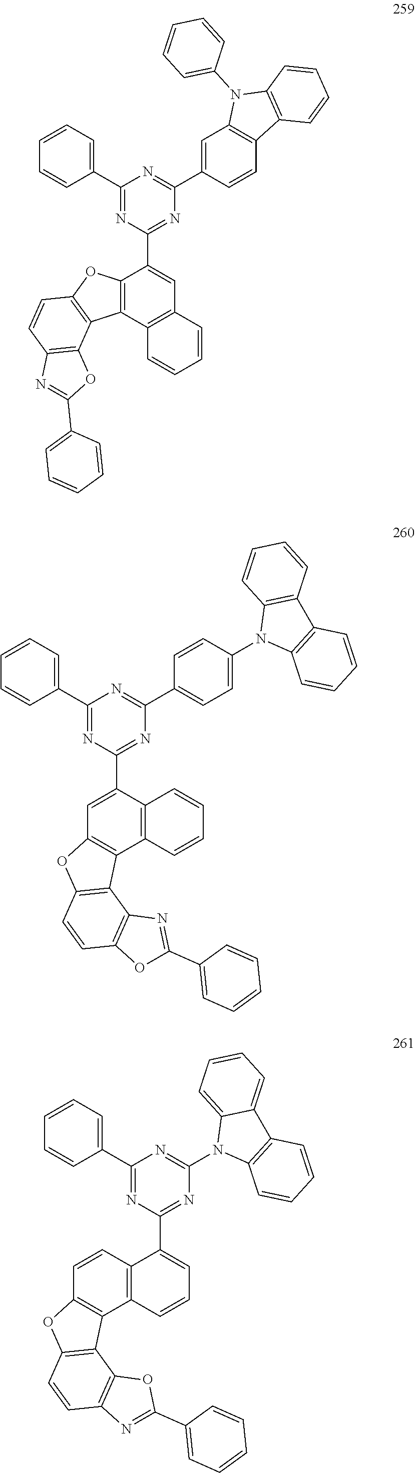

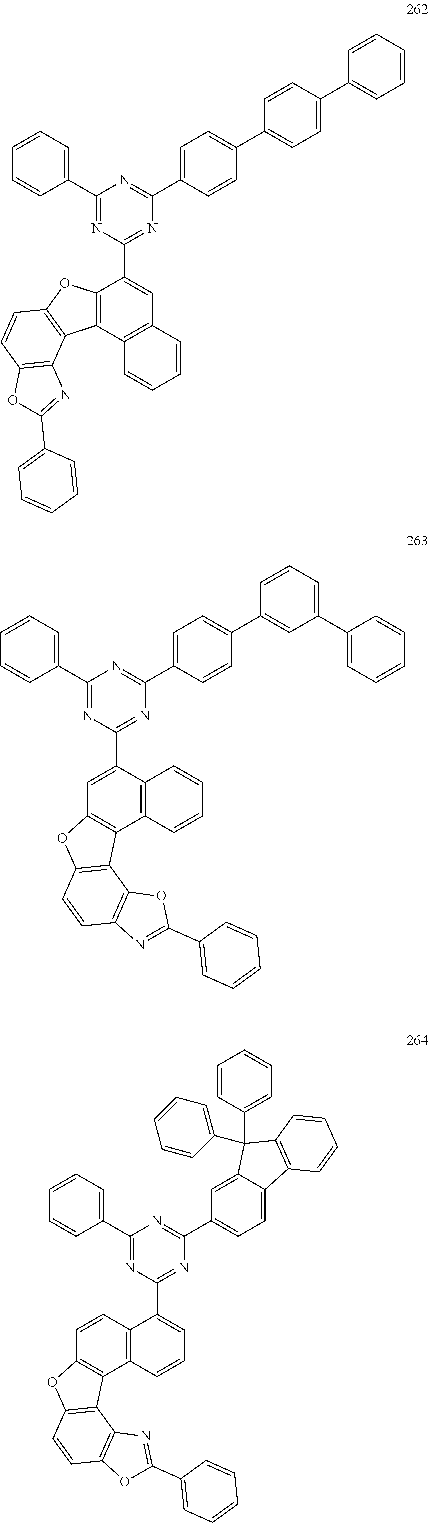 [SMILES: c1ccc(-c2ccc(-c3ccc(-c4nc(-c5ccccc5)nc(-c5cc6ccccc6c6c5oc5ccc7oc(-c8ccccc8)nc7c56)n4)cc3)cc2)cc1 ; c1ccc(-c2cccc(-c3ccc(-c4nc(-c5ccccc5)nc(-c5cc6oc7ccc8nc(-c9ccccc9)oc8c7c6c6ccccc56)n4)cc3)c2)cc1 ; c1ccc(-c2nc(-c3ccc4c(c3)C(c3ccccc3)(c3ccccc3)c3ccccc3-4)nc(-c3cccc4c3ccc3oc5ccc6oc(-c7ccccc7)nc6c5c34)n2)cc1]

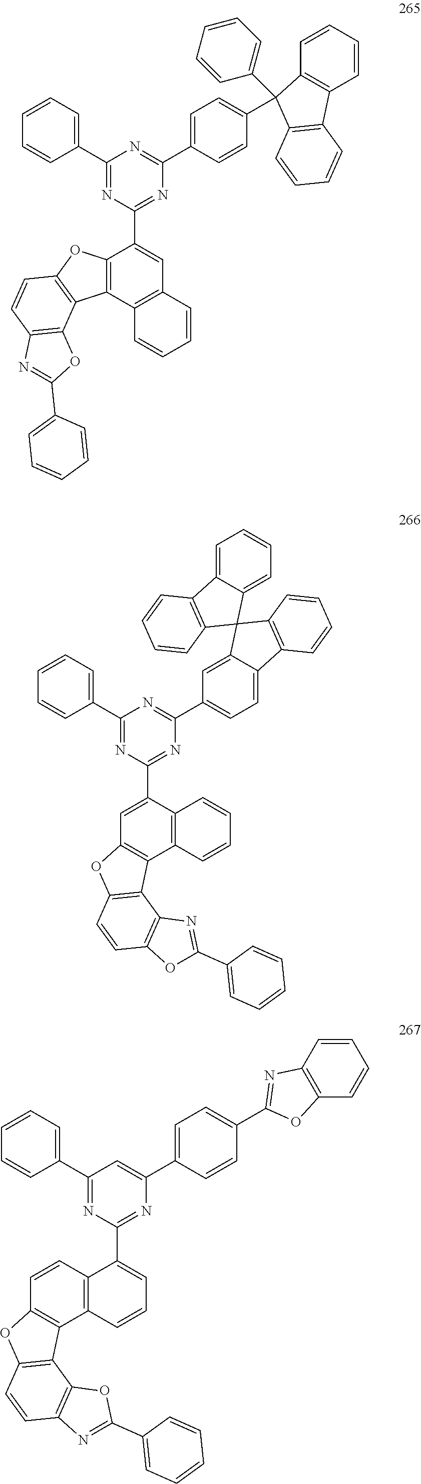 [SMILES: c1ccc(-c2nc(-c3ccc(C4(c5ccccc5)c5ccccc5-c5ccccc54)cc3)nc(-c3cc4ccccc4c4c3oc3ccc5nc(-c6ccccc6)oc5c34)n2)cc1 ; c1ccc(-c2nc(-c3ccc4c(c3)C3(c5ccccc5-c5ccccc53)c3ccccc3-4)nc(-c3cc4oc5ccc6oc(-c7ccccc7)nc6c5c4c4ccccc34)n2)cc1 ; c1ccc(-c2cc(-c3ccc(-c4nc5ccccc5o4)cc3)nc(-c3cccc4c3ccc3oc5ccc6nc(-c7ccccc7)oc6c5c34)n2)cc1]

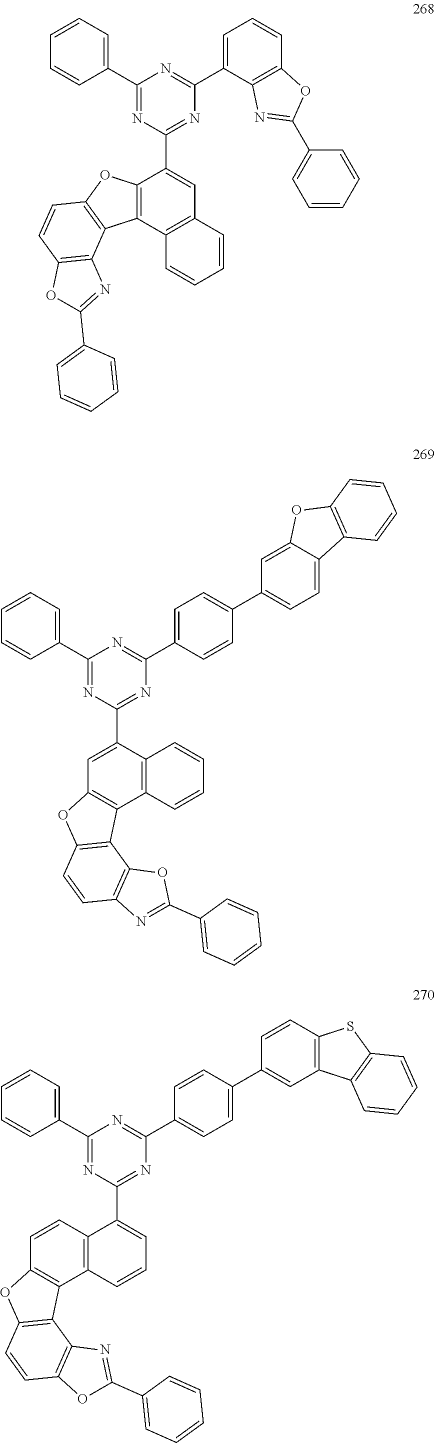 [SMILES: c1ccc(-c2nc(-c3cccc4oc(-c5ccccc5)nc34)nc(-c3cc4ccccc4c4c3oc3ccc5oc(-c6ccccc6)nc5c34)n2)cc1 ; c1ccc(-c2nc(-c3ccc(-c4ccc5c(c4)oc4ccccc45)cc3)nc(-c3cc4oc5ccc6nc(-c7ccccc7)oc6c5c4c4ccccc34)n2)cc1 ; c1ccc(-c2nc(-c3ccc(-c4ccc5sc6ccccc6c5c4)cc3)nc(-c3cccc4c3ccc3oc5ccc6oc(-c7ccccc7)nc6c5c34)n2)cc1]

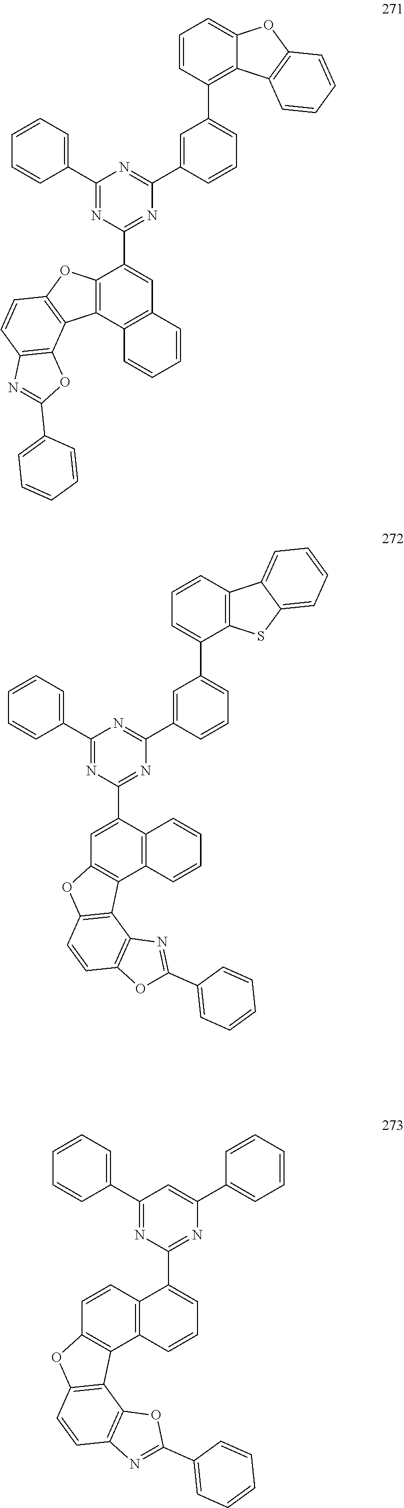 [SMILES: c1ccc(-c2nc(-c3cccc(-c4cccc5oc6ccccc6c45)c3)nc(-c3cc4ccccc4c4c3oc3ccc5nc(-c6ccccc6)oc5c34)n2)cc1 ; c1ccc(-c2nc(-c3cccc(-c4cccc5c4sc4ccccc45)c3)nc(-c3cc4oc5ccc6oc(-c7ccccc7)nc6c5c4c4ccccc34)n2)cc1 ; c1ccc(-c2cc(-c3ccccc3)nc(-c3cccc4c3ccc3oc5ccc6nc(-c7ccccc7)oc6c5c34)n2)cc1]

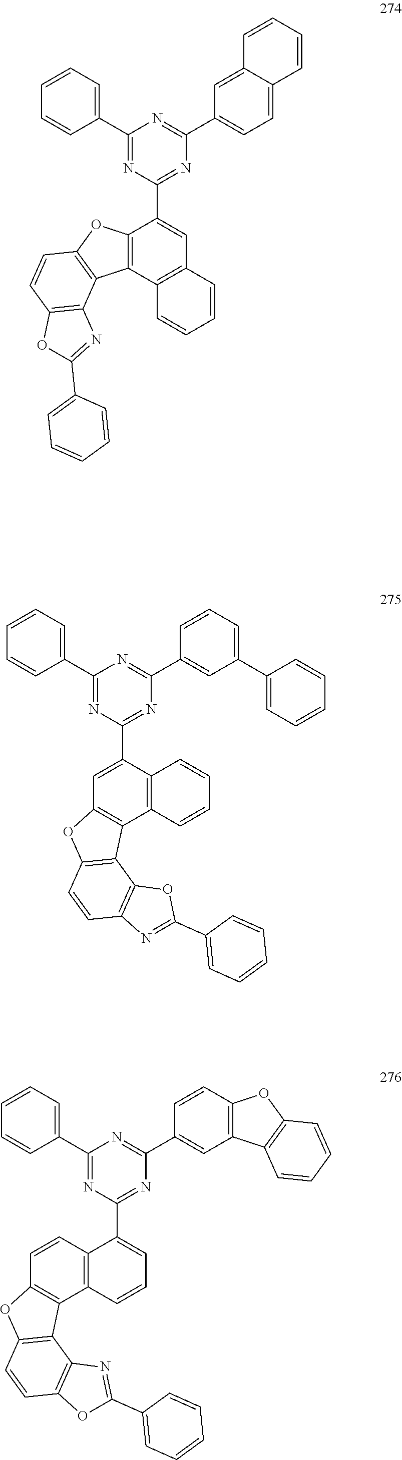 [SMILES: c1ccc(-c2nc(-c3ccc4ccccc4c3)nc(-c3cc4ccccc4c4c3oc3ccc5oc(-c6ccccc6)nc5c34)n2)cc1 ; c1ccc(-c2cccc(-c3nc(-c4ccccc4)nc(-c4cc5oc6ccc7nc(-c8ccccc8)oc7c6c5c5ccccc45)n3)c2)cc1 ; c1ccc(-c2nc(-c3ccc4oc5ccccc5c4c3)nc(-c3cccc4c3ccc3oc5ccc6oc(-c7ccccc7)nc6c5c34)n2)cc1]

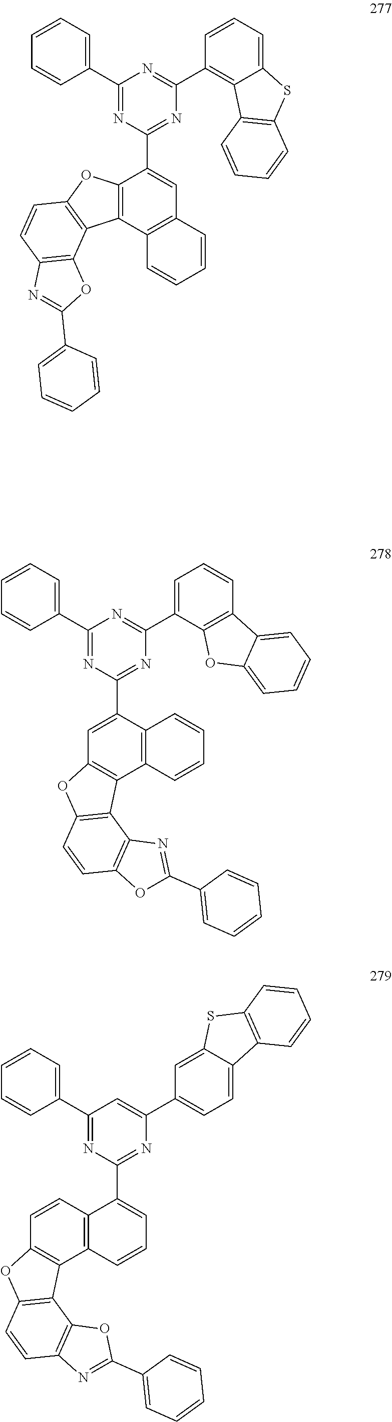 [SMILES: c1ccc(-c2nc(-c3cc4ccccc4c4c3oc3ccc5nc(-c6ccccc6)oc5c34)nc(-c3cccc4sc5ccccc5c34)n2)cc1 ; c1ccc(-c2nc(-c3cc4oc5ccc6oc(-c7ccccc7)nc6c5c4c4ccccc34)nc(-c3cccc4c3oc3ccccc34)n2)cc1 ; c1ccc(-c2cc(-c3ccc4c(c3)sc3ccccc34)nc(-c3cccc4c3ccc3oc5ccc6nc(-c7ccccc7)oc6c5c34)n2)cc1]

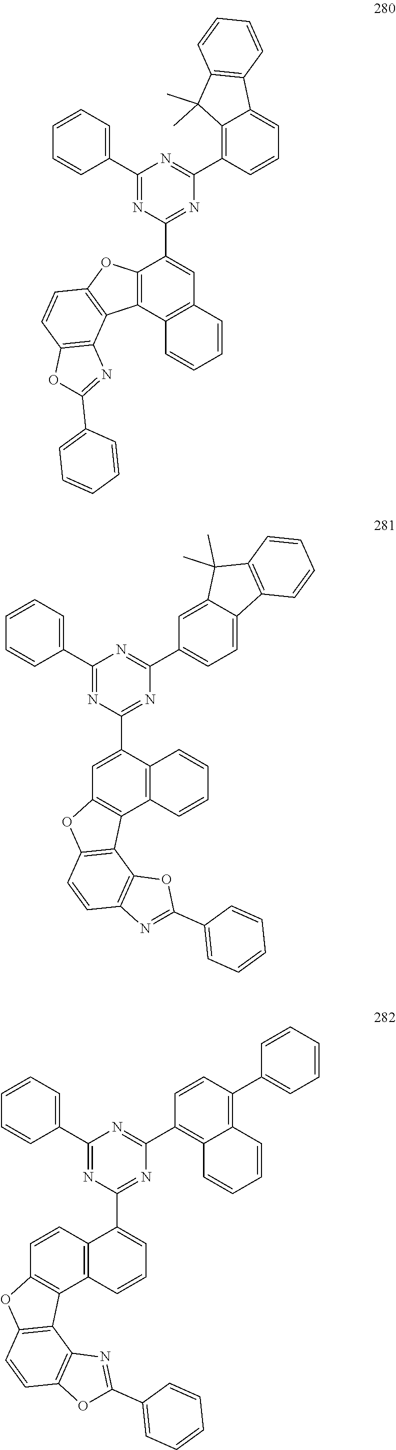 [SMILES: CC1(C)c2ccccc2-c2cccc(-c3nc(-c4ccccc4)nc(-c4cc5ccccc5c5c4oc4ccc6oc(-c7ccccc7)nc6c45)n3)c21 ; CC1(C)c2ccccc2-c2ccc(-c3nc(-c4ccccc4)nc(-c4cc5oc6ccc7nc(-c8ccccc8)oc7c6c5c5ccccc45)n3)cc21 ; c1ccc(-c2nc(-c3ccc(-c4ccccc4)c4ccccc34)nc(-c3cccc4c3ccc3oc5ccc6oc(-c7ccccc7)nc6c5c34)n2)cc1]

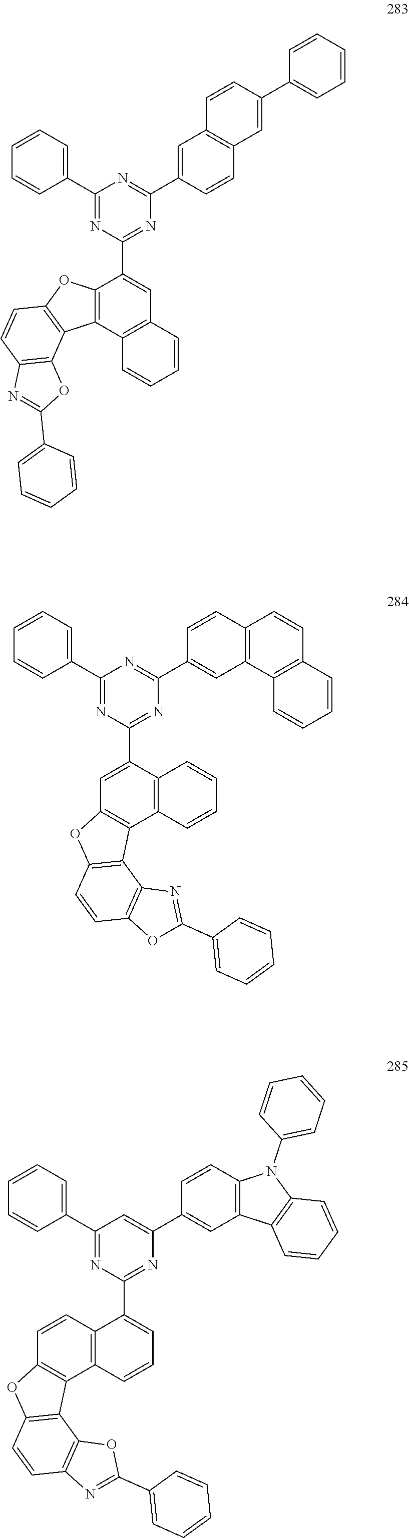 [SMILES: c1ccc(-c2ccc3cc(-c4nc(-c5ccccc5)nc(-c5cc6ccccc6c6c5oc5ccc7nc(-c8ccccc8)oc7c56)n4)ccc3c2)cc1 ; c1ccc(-c2nc(-c3ccc4ccc5ccccc5c4c3)nc(-c3cc4oc5ccc6oc(-c7ccccc7)nc6c5c4c4ccccc34)n2)cc1 ; c1ccc(-c2cc(-c3ccc4c(c3)c3ccccc3n4-c3ccccc3)nc(-c3cccc4c3ccc3oc5ccc6nc(-c7ccccc7)oc6c5c34)n2)cc1]

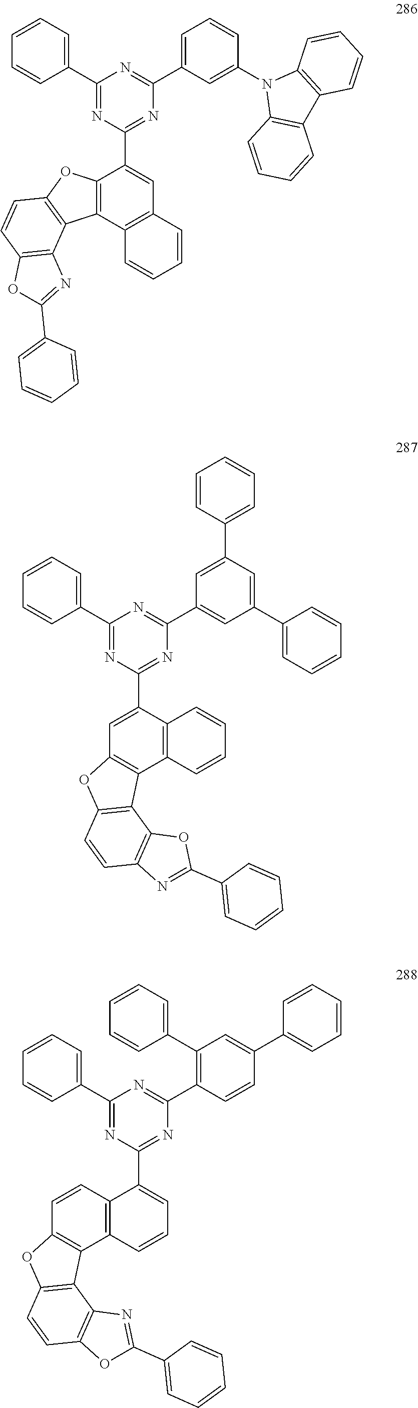 [SMILES: c1ccc(-c2nc(-c3cccc(-n4c5ccccc5c5ccccc54)c3)nc(-c3cc4ccccc4c4c3oc3ccc5oc(-c6ccccc6)nc5c34)n2)cc1 ; c1ccc(-c2cc(-c3ccccc3)cc(-c3nc(-c4ccccc4)nc(-c4cc5oc6ccc7nc(-c8ccccc8)oc7c6c5c5ccccc45)n3)c2)cc1 ; c1ccc(-c2ccc(-c3nc(-c4ccccc4)nc(-c4cccc5c4ccc4oc6ccc7oc(-c8ccccc8)nc7c6c45)n3)c(-c3ccccc3)c2)cc1]

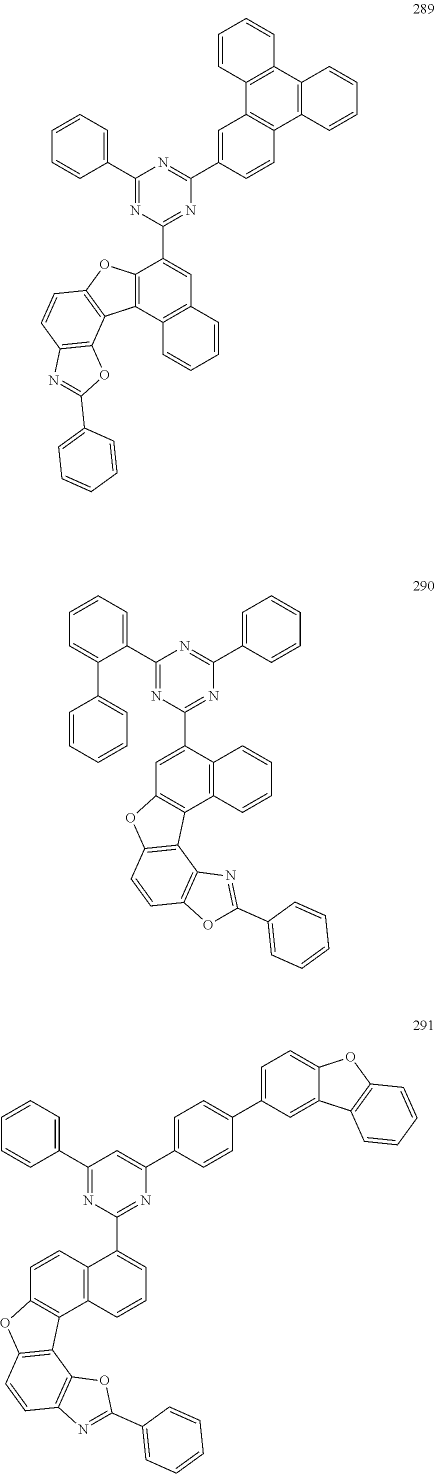 [SMILES: c1ccc(-c2nc(-c3ccc4c5ccccc5c5ccccc5c4c3)nc(-c3cc4ccccc4c4c3oc3ccc5nc(-c6ccccc6)oc5c34)n2)cc1 ; c1ccc(-c2nc(-c3ccccc3-c3ccccc3)nc(-c3cc4oc5ccc6oc(-c7ccccc7)nc6c5c4c4ccccc34)n2)cc1 ; c1ccc(-c2cc(-c3ccc(-c4ccc5oc6ccccc6c5c4)cc3)nc(-c3cccc4c3ccc3oc5ccc6nc(-c7ccccc7)oc6c5c34)n2)cc1]

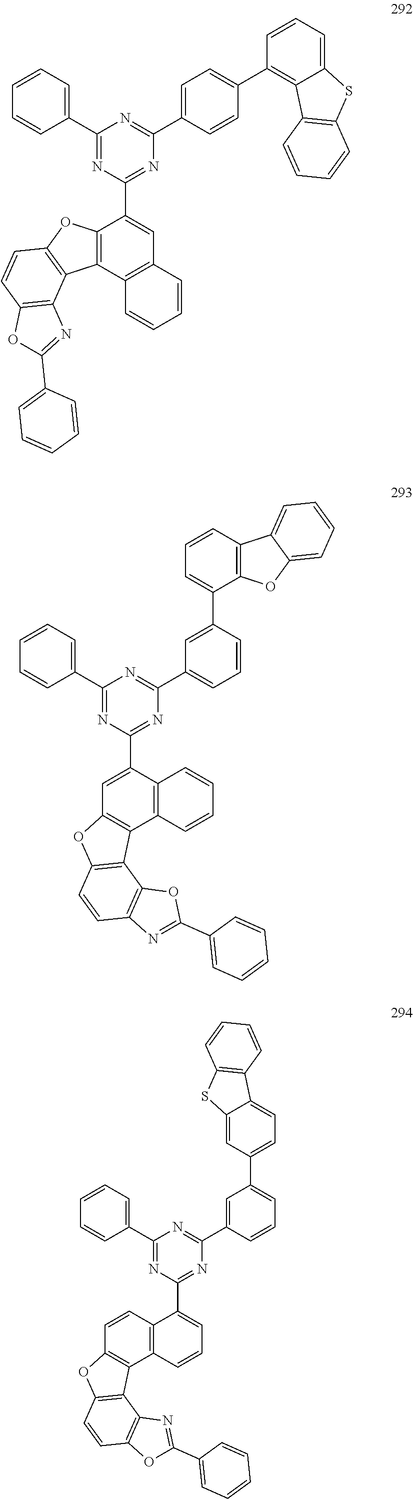 [SMILES: c1ccc(-c2nc(-c3ccc(-c4cccc5sc6ccccc6c45)cc3)nc(-c3cc4ccccc4c4c3oc3ccc5oc(-c6ccccc6)nc5c34)n2)cc1 ; c1ccc(-c2nc(-c3cccc(-c4cccc5c4oc4ccccc45)c3)nc(-c3cc4oc5ccc6nc(-c7ccccc7)oc6c5c4c4ccccc34)n2)cc1 ; c1ccc(-c2nc(-c3cccc(-c4ccc5c(c4)sc4ccccc45)c3)nc(-c3cccc4c3ccc3oc5ccc6oc(-c7ccccc7)nc6c5c34)n2)cc1]

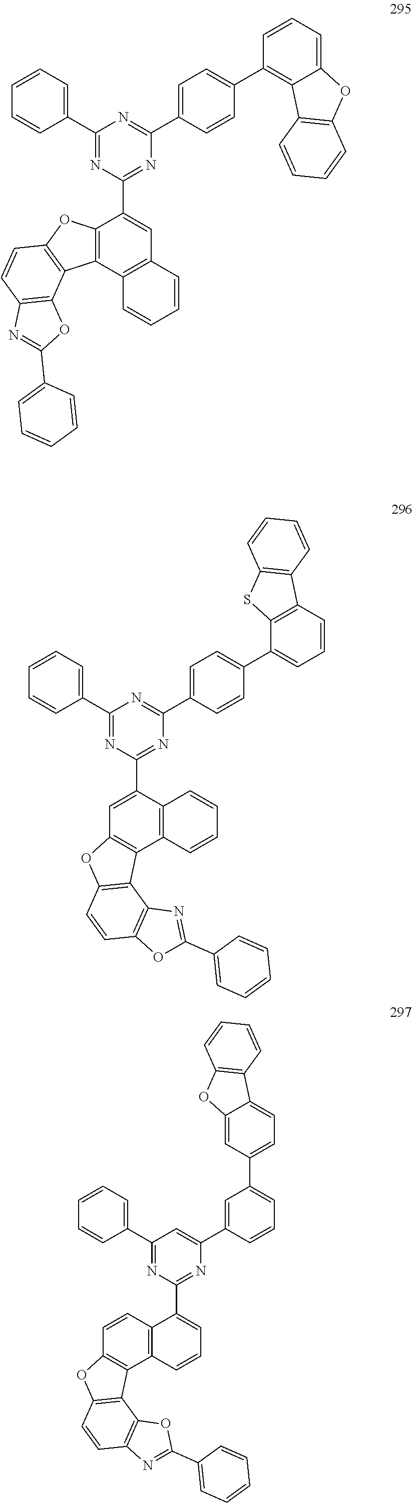

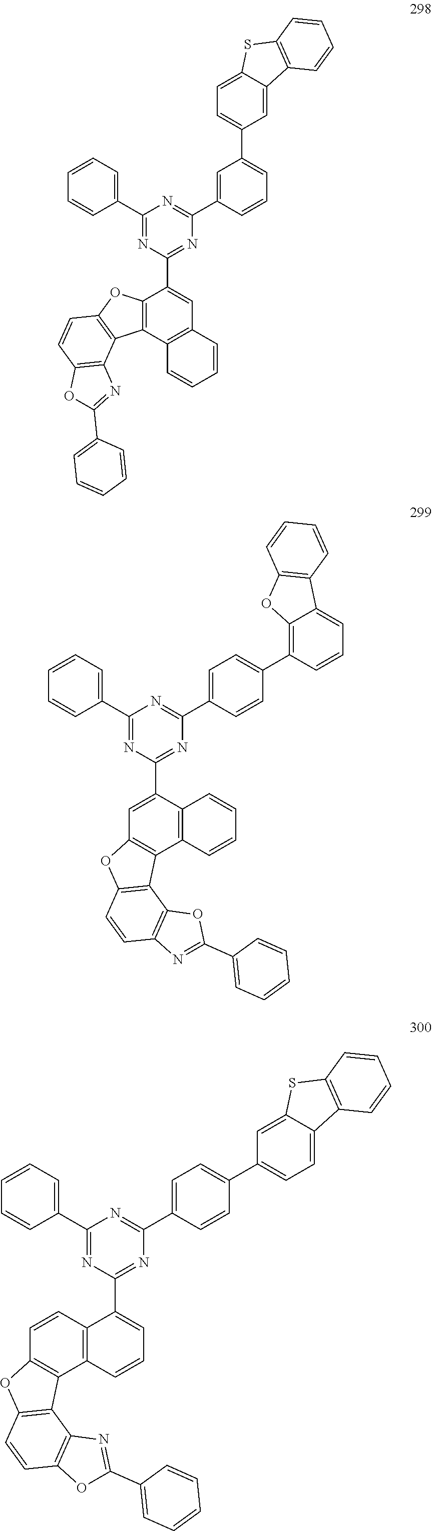 [SMILES: c1ccc(-c2nc(-c3cccc(-c4ccc5sc6ccccc6c5c4)c3)nc(-c3cc4ccccc4c4c3oc3ccc5oc(-c6ccccc6)nc5c34)n2)cc1 ; c1ccc(-c2nc(-c3ccc(-c4cccc5c4oc4ccccc45)cc3)nc(-c3cc4oc5ccc6nc(-c7ccccc7)oc6c5c4c4ccccc34)n2)cc1 ; c1ccc(-c2nc(-c3ccc(-c4ccc5c(c4)sc4ccccc45)cc3)nc(-c3cccc4c3ccc3oc5ccc6oc(-c7ccccc7)nc6c5c34)n2)cc1]

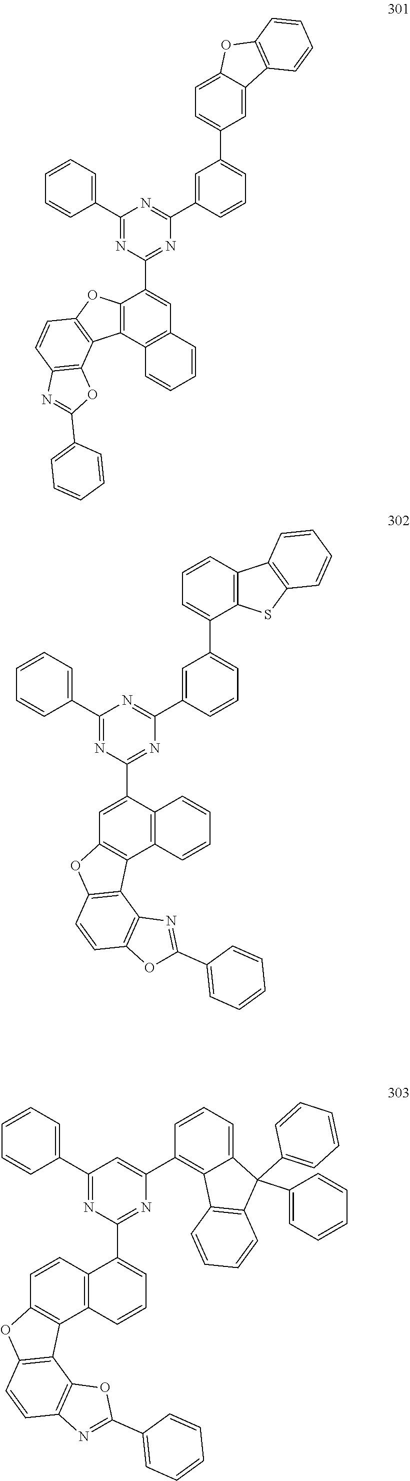 [SMILES: c1ccc(-c2nc(-c3cccc(-c4ccc5oc6ccccc6c5c4)c3)nc(-c3cc4ccccc4c4c3oc3ccc5nc(-c6ccccc6)oc5c34)n2)cc1 ; c1ccc(-c2nc(-c3cccc(-c4cccc5c4sc4ccccc45)c3)nc(-c3cc4oc5ccc6oc(-c7ccccc7)nc6c5c4c4ccccc34)n2)cc1 ; c1ccc(-c2cc(-c3cccc4c3-c3ccccc3C4(c3ccccc3)c3ccccc3)nc(-c3cccc4c3ccc3oc5ccc6nc(-c7ccccc7)oc6c5c34)n2)cc1]

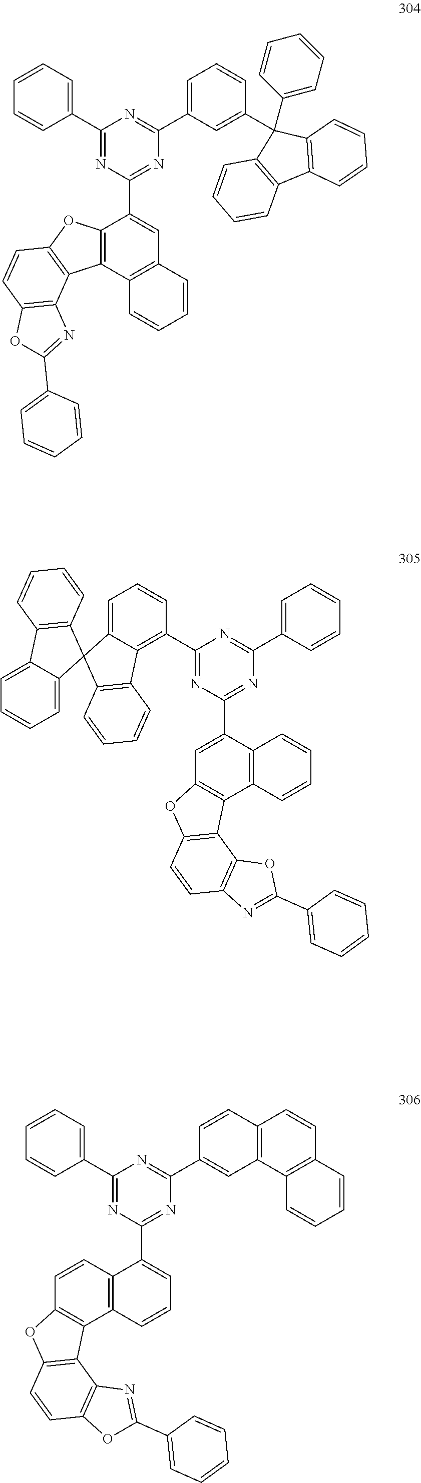 [SMILES: c1ccc(-c2nc(-c3cccc(C4(c5ccccc5)c5ccccc5-c5ccccc54)c3)nc(-c3cc4ccccc4c4c3oc3ccc5oc(-c6ccccc6)nc5c34)n2)cc1 ; c1ccc(-c2nc(-c3cccc4c3-c3ccccc3C43c4ccccc4-c4ccccc43)nc(-c3cc4oc5ccc6nc(-c7ccccc7)oc6c5c4c4ccccc34)n2)cc1 ; c1ccc(-c2nc(-c3ccc4ccc5ccccc5c4c3)nc(-c3cccc4c3ccc3oc5ccc6oc(-c7ccccc7)nc6c5c34)n2)cc1]

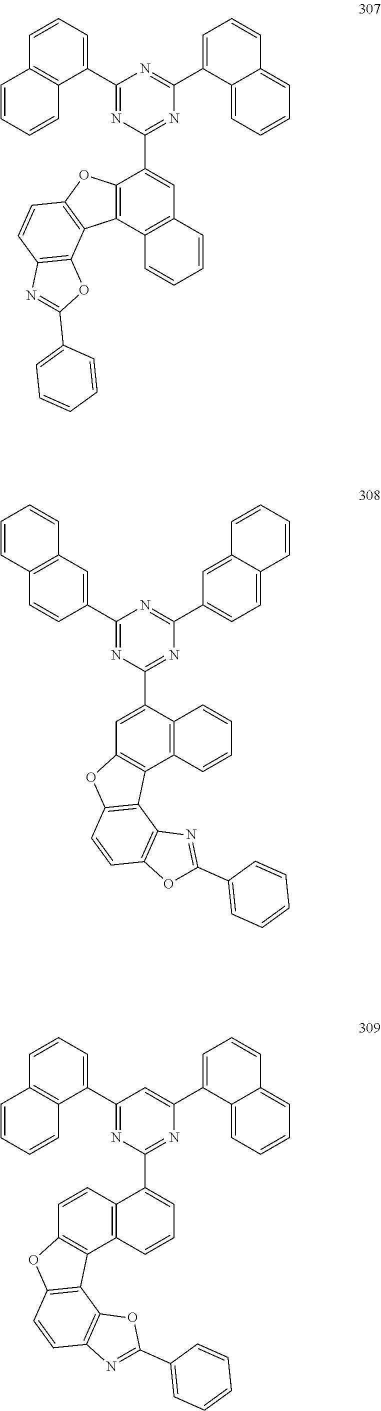 [SMILES: c1ccc(-c2nc3ccc4oc5c(-c6nc(-c7cccc8ccccc78)nc(-c7cccc8ccccc78)n6)cc6ccccc6c5c4c3o2)cc1 ; c1ccc(-c2nc3c(ccc4oc5cc(-c6nc(-c7ccc8ccccc8c7)nc(-c7ccc8ccccc8c7)n6)c6ccccc6c5c43)o2)cc1 ; c1ccc(-c2nc3ccc4oc5ccc6c(-c7nc(-c8cccc9ccccc89)cc(-c8cccc9ccccc89)n7)cccc6c5c4c3o2)cc1]

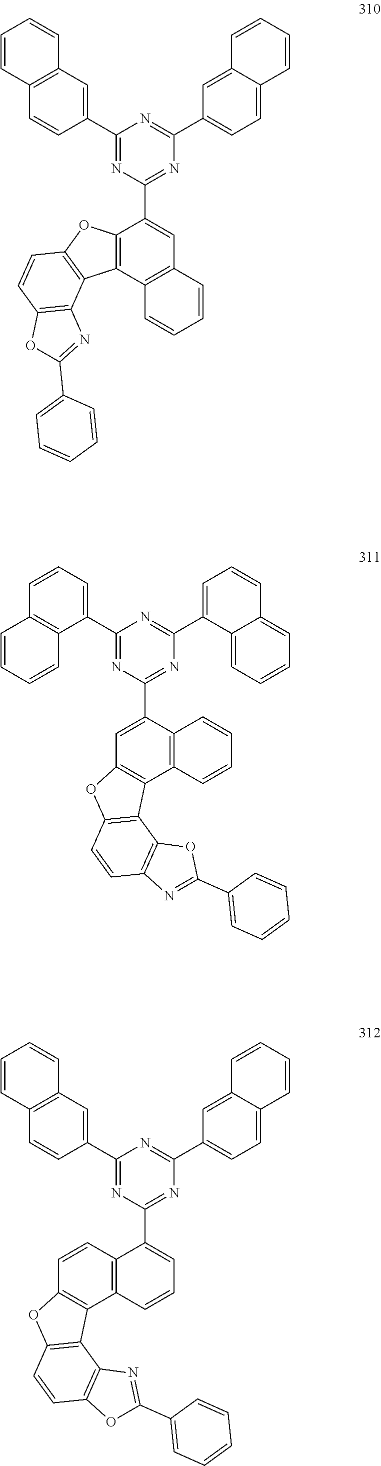 [SMILES: c1ccc(-c2nc3c(ccc4oc5c(-c6nc(-c7ccc8ccccc8c7)nc(-c7ccc8ccccc8c7)n6)cc6ccccc6c5c43)o2)cc1 ; c1ccc(-c2nc3ccc4oc5cc(-c6nc(-c7cccc8ccccc78)nc(-c7cccc8ccccc78)n6)c6ccccc6c5c4c3o2)cc1 ; c1ccc(-c2nc3c(ccc4oc5ccc6c(-c7nc(-c8ccc9ccccc9c8)nc(-c8ccc9ccccc9c8)n7)cccc6c5c43)o2)cc1]

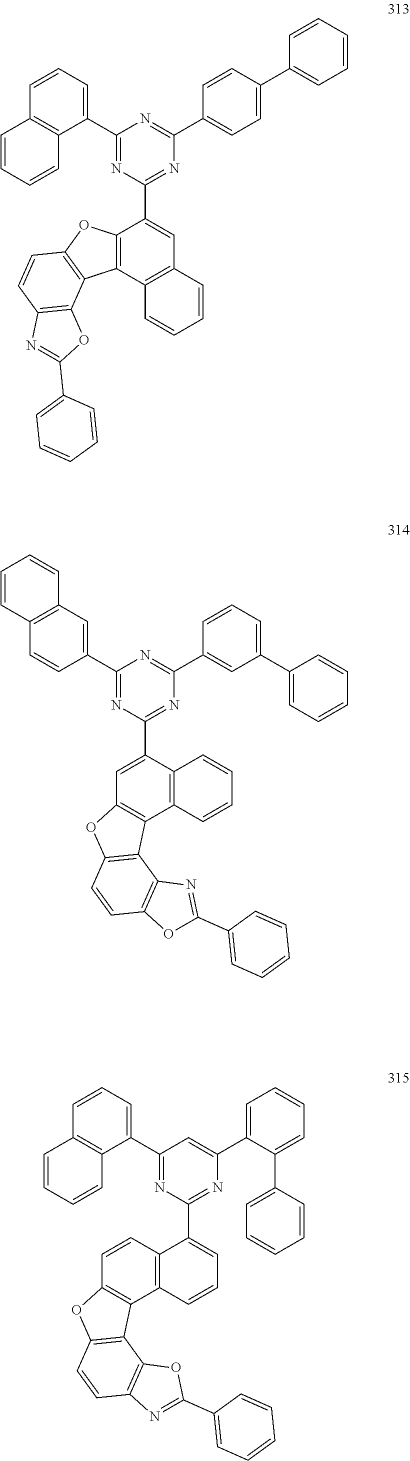 [SMILES: c1ccc(-c2ccc(-c3nc(-c4cccc5ccccc45)nc(-c4cc5ccccc5c5c4oc4ccc6nc(-c7ccccc7)oc6c45)n3)cc2)cc1 ; c1ccc(-c2cccc(-c3nc(-c4ccc5ccccc5c4)nc(-c4cc5oc6ccc7oc(-c8ccccc8)nc7c6c5c5ccccc45)n3)c2)cc1 ; c1ccc(-c2nc3ccc4oc5ccc6c(-c7nc(-c8ccccc8-c8ccccc8)cc(-c8cccc9ccccc89)n7)cccc6c5c4c3o2)cc1]

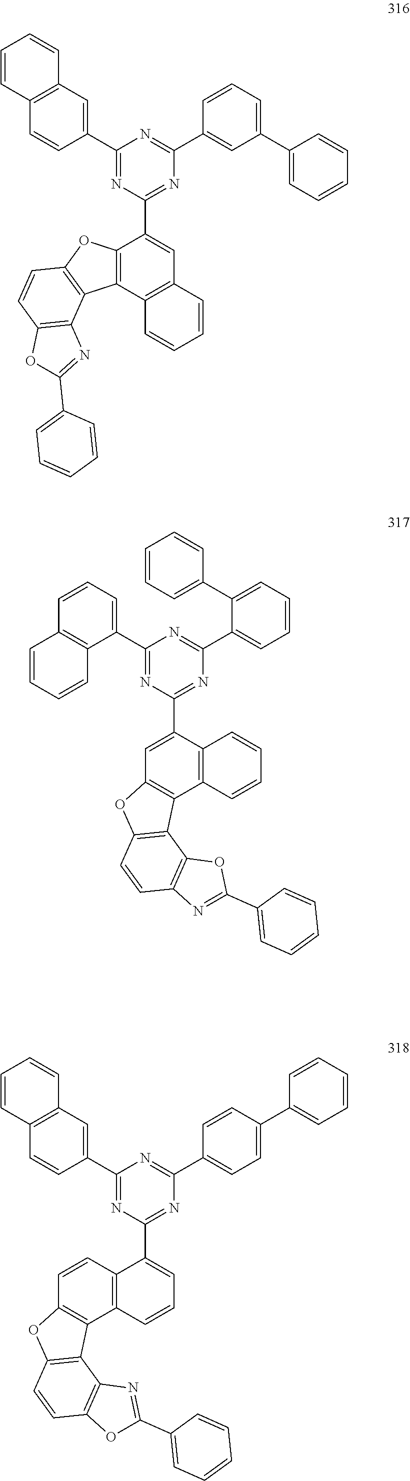 [SMILES: c1ccc(-c2cccc(-c3nc(-c4ccc5ccccc5c4)nc(-c4cc5ccccc5c5c4oc4ccc6oc(-c7ccccc7)nc6c45)n3)c2)cc1 ; c1ccc(-c2nc3ccc4oc5cc(-c6nc(-c7ccccc7-c7ccccc7)nc(-c7cccc8ccccc78)n6)c6ccccc6c5c4c3o2)cc1 ; c1ccc(-c2ccc(-c3nc(-c4ccc5ccccc5c4)nc(-c4cccc5c4ccc4oc6ccc7oc(-c8ccccc8)nc7c6c45)n3)cc2)cc1]

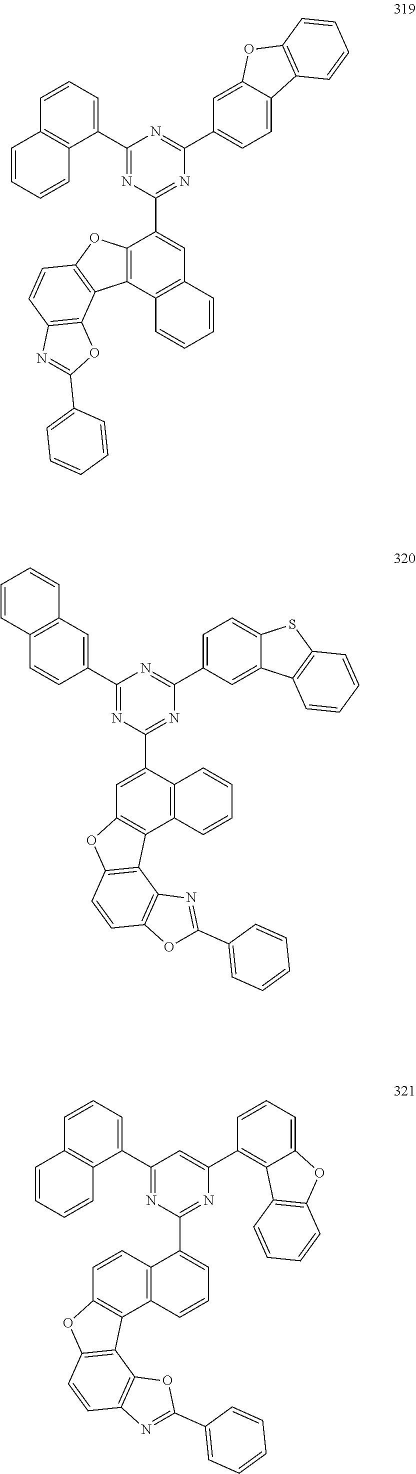 [SMILES: c1ccc(-c2nc3ccc4oc5c(-c6nc(-c7ccc8c(c7)oc7ccccc78)nc(-c7cccc8ccccc78)n6)cc6ccccc6c5c4c3o2)cc1 ; c1ccc(-c2nc3c(ccc4oc5cc(-c6nc(-c7ccc8ccccc8c7)nc(-c7ccc8sc9ccccc9c8c7)n6)c6ccccc6c5c43)o2)cc1 ; c1ccc(-c2nc3ccc4oc5ccc6c(-c7nc(-c8cccc9ccccc89)cc(-c8cccc9oc%10ccccc%10c89)n7)cccc6c5c4c3o2)cc1]

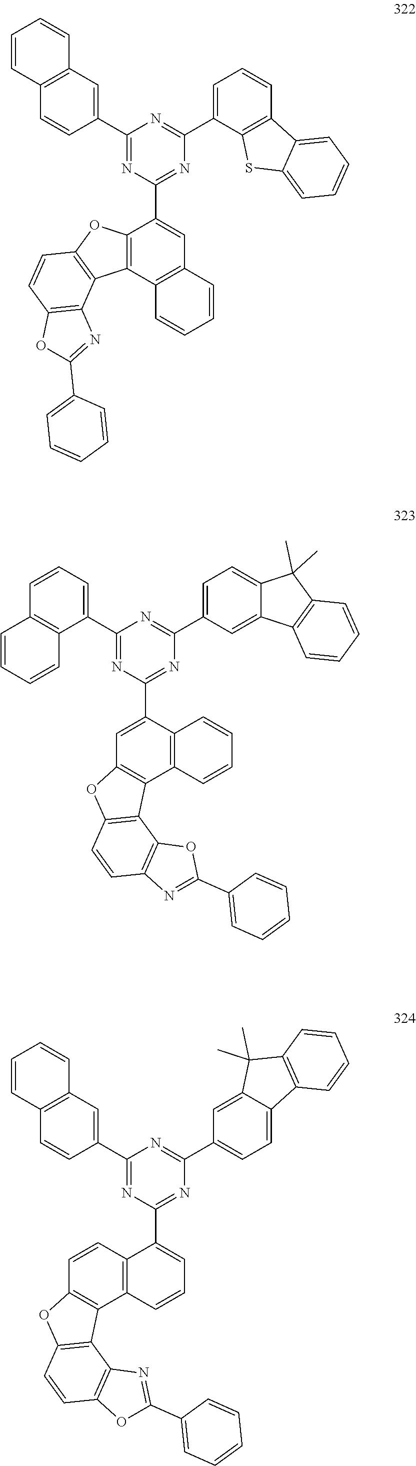 [SMILES: c1ccc(-c2nc3c(ccc4oc5c(-c6nc(-c7ccc8ccccc8c7)nc(-c7cccc8c7sc7ccccc78)n6)cc6ccccc6c5c43)o2)cc1 ; CC1(C)c2ccccc2-c2cc(-c3nc(-c4cccc5ccccc45)nc(-c4cc5oc6ccc7nc(-c8ccccc8)oc7c6c5c5ccccc45)n3)ccc21 ; CC1(C)c2ccccc2-c2ccc(-c3nc(-c4ccc5ccccc5c4)nc(-c4cccc5c4ccc4oc6ccc7oc(-c8ccccc8)nc7c6c45)n3)cc21]

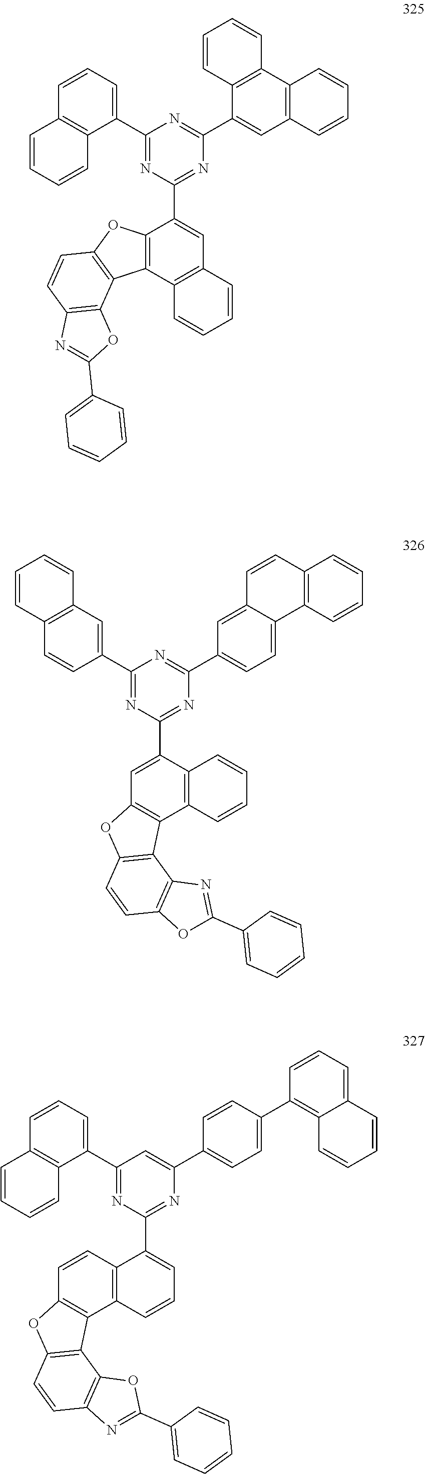 [SMILES: c1ccc(-c2nc3ccc4oc5c(-c6nc(-c7cccc8ccccc78)nc(-c7cc8ccccc8c8ccccc78)n6)cc6ccccc6c5c4c3o2)cc1 ; c1ccc(-c2nc3c(ccc4oc5cc(-c6nc(-c7ccc8ccccc8c7)nc(-c7ccc8c(ccc9ccccc98)c7)n6)c6ccccc6c5c43)o2)cc1 ; c1ccc(-c2nc3ccc4oc5ccc6c(-c7nc(-c8ccc(-c9cccc%10ccccc9%10)cc8)cc(-c8cccc9ccccc89)n7)cccc6c5c4c3o2)cc1]

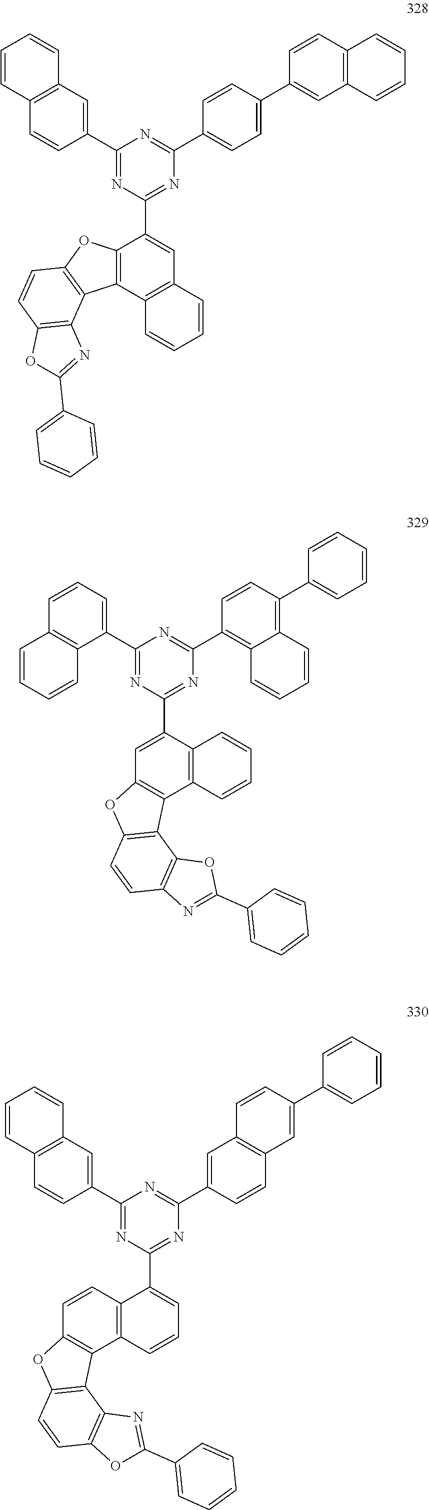 [SMILES: c1ccc(-c2nc3c(ccc4oc5c(-c6nc(-c7ccc(-c8ccc9ccccc9c8)cc7)nc(-c7ccc8ccccc8c7)n6)cc6ccccc6c5c43)o2)cc1 ; c1ccc(-c2nc3ccc4oc5cc(-c6nc(-c7cccc8ccccc78)nc(-c7ccc(-c8ccccc8)c8ccccc78)n6)c6ccccc6c5c4c3o2)cc1 ; c1ccc(-c2ccc3cc(-c4nc(-c5ccc6ccccc6c5)nc(-c5cccc6c5ccc5oc7ccc8oc(-c9ccccc9)nc8c7c56)n4)ccc3c2)cc1]

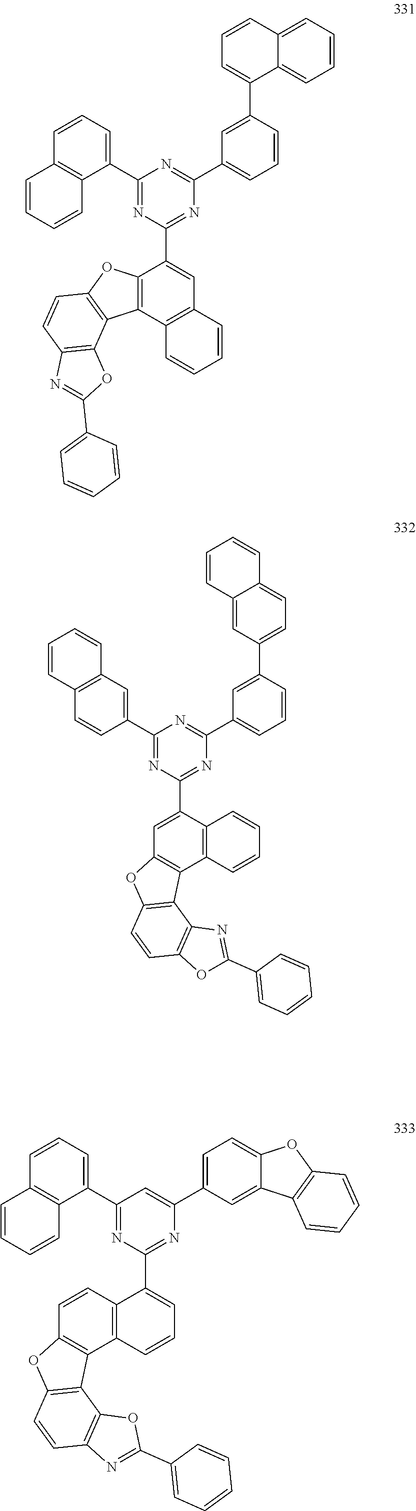 [SMILES: c1ccc(-c2nc3ccc4oc5c(-c6nc(-c7cccc(-c8cccc9ccccc89)c7)nc(-c7cccc8ccccc78)n6)cc6ccccc6c5c4c3o2)cc1 ; c1ccc(-c2nc3c(ccc4oc5cc(-c6nc(-c7cccc(-c8ccc9ccccc9c8)c7)nc(-c7ccc8ccccc8c7)n6)c6ccccc6c5c43)o2)cc1 ; c1ccc(-c2nc3ccc4oc5ccc6c(-c7nc(-c8ccc9oc%10ccccc%10c9c8)cc(-c8cccc9ccccc89)n7)cccc6c5c4c3o2)cc1]

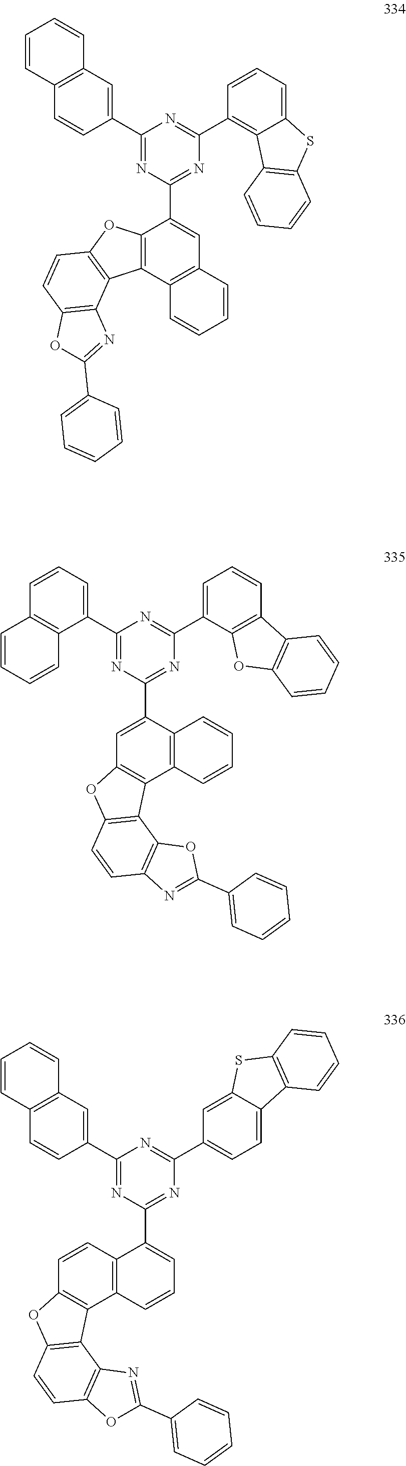 [SMILES: c1ccc(-c2nc3c(ccc4oc5c(-c6nc(-c7ccc8ccccc8c7)nc(-c7cccc8sc9ccccc9c78)n6)cc6ccccc6c5c43)o2)cc1 ; c1ccc(-c2nc3ccc4oc5cc(-c6nc(-c7cccc8ccccc78)nc(-c7cccc8c7oc7ccccc78)n6)c6ccccc6c5c4c3o2)cc1 ; c1ccc(-c2nc3c(ccc4oc5ccc6c(-c7nc(-c8ccc9ccccc9c8)nc(-c8ccc9c(c8)sc8ccccc89)n7)cccc6c5c43)o2)cc1]

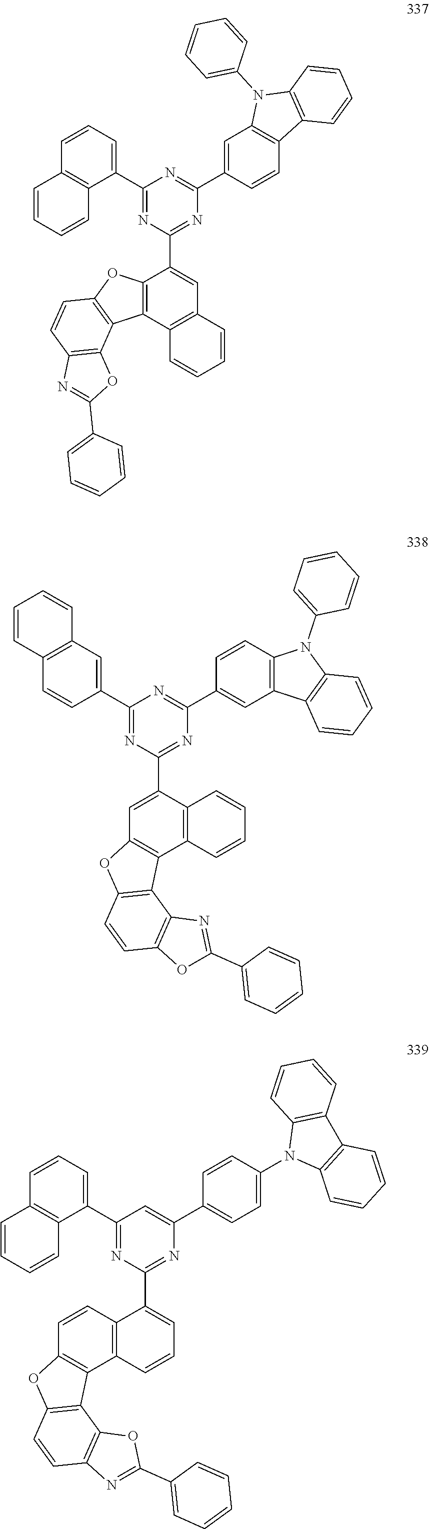 [SMILES: c1ccc(-c2nc3ccc4oc5c(-c6nc(-c7ccc8c9ccccc9n(-c9ccccc9)c8c7)nc(-c7cccc8ccccc78)n6)cc6ccccc6c5c4c3o2)cc1 ; c1ccc(-c2nc3c(ccc4oc5cc(-c6nc(-c7ccc8ccccc8c7)nc(-c7ccc8c(c7)c7ccccc7n8-c7ccccc7)n6)c6ccccc6c5c43)o2)cc1 ; c1ccc(-c2nc3ccc4oc5ccc6c(-c7nc(-c8ccc(-n9c%10ccccc%10c%10ccccc%109)cc8)cc(-c8cccc9ccccc89)n7)cccc6c5c4c3o2)cc1]

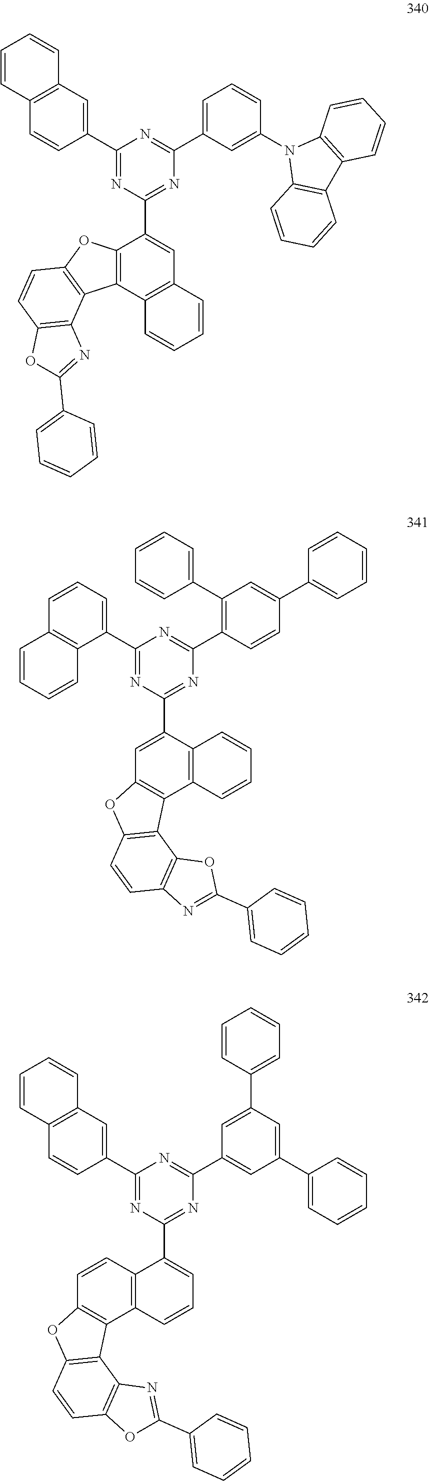 [SMILES: c1ccc(-c2nc3c(ccc4oc5c(-c6nc(-c7cccc(-n8c9ccccc9c9ccccc98)c7)nc(-c7ccc8ccccc8c7)n6)cc6ccccc6c5c43)o2)cc1 ; c1ccc(-c2ccc(-c3nc(-c4cccc5ccccc45)nc(-c4cc5oc6ccc7nc(-c8ccccc8)oc7c6c5c5ccccc45)n3)c(-c3ccccc3)c2)cc1 ; c1ccc(-c2cc(-c3ccccc3)cc(-c3nc(-c4ccc5ccccc5c4)nc(-c4cccc5c4ccc4oc6ccc7oc(-c8ccccc8)nc7c6c45)n3)c2)cc1]

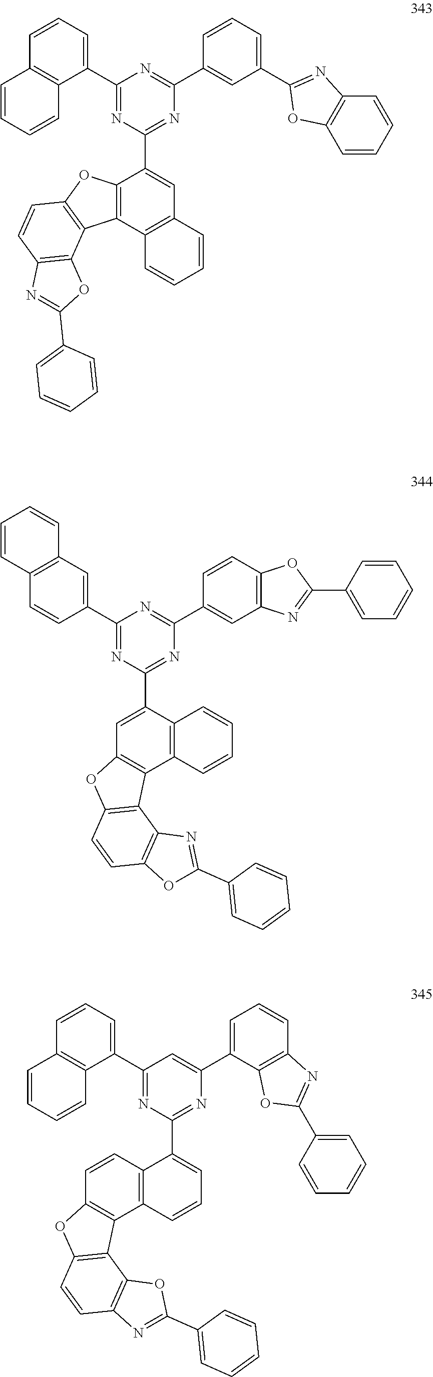 [SMILES: c1ccc(-c2nc3ccc4oc5c(-c6nc(-c7cccc(-c8nc9ccccc9o8)c7)nc(-c7cccc8ccccc78)n6)cc6ccccc6c5c4c3o2)cc1 ; c1ccc(-c2nc3cc(-c4nc(-c5ccc6ccccc6c5)nc(-c5cc6oc7ccc8oc(-c9ccccc9)nc8c7c6c6ccccc56)n4)ccc3o2)cc1 ; c1ccc(-c2nc3cccc(-c4cc(-c5cccc6ccccc56)nc(-c5cccc6c5ccc5oc7ccc8nc(-c9ccccc9)oc8c7c56)n4)c3o2)cc1]

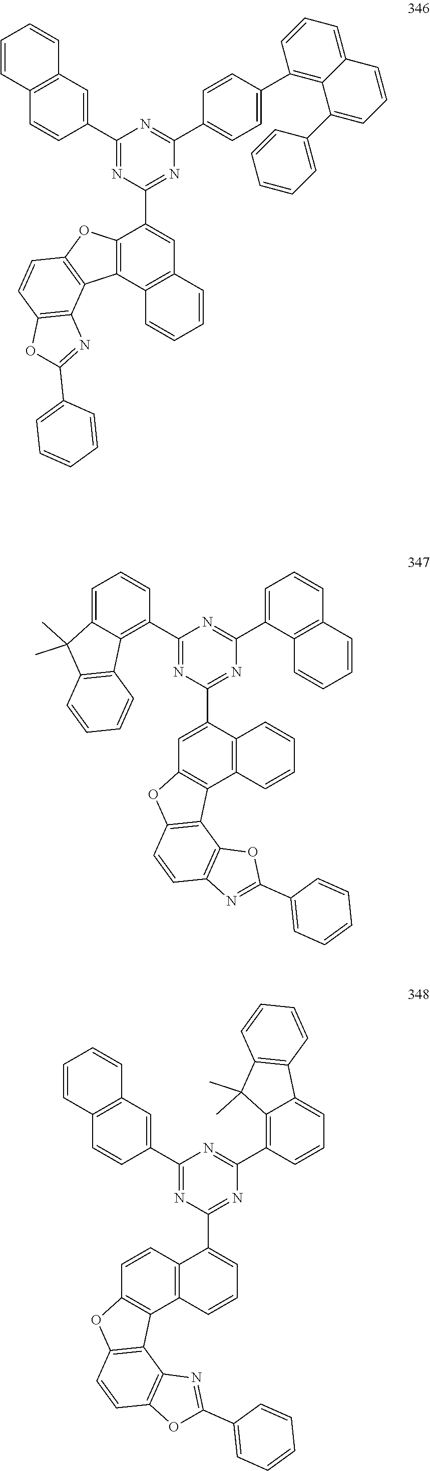 [SMILES: c1ccc(-c2nc3c(ccc4oc5c(-c6nc(-c7ccc(-c8cccc9cccc(-c%10ccccc%10)c89)cc7)nc(-c7ccc8ccccc8c7)n6)cc6ccccc6c5c43)o2)cc1 ; CC1(C)c2ccccc2-c2c(-c3nc(-c4cccc5ccccc45)nc(-c4cc5oc6ccc7nc(-c8ccccc8)oc7c6c5c5ccccc45)n3)cccc21 ; CC1(C)c2ccccc2-c2cccc(-c3nc(-c4ccc5ccccc5c4)nc(-c4cccc5c4ccc4oc6ccc7oc(-c8ccccc8)nc7c6c45)n3)c21]

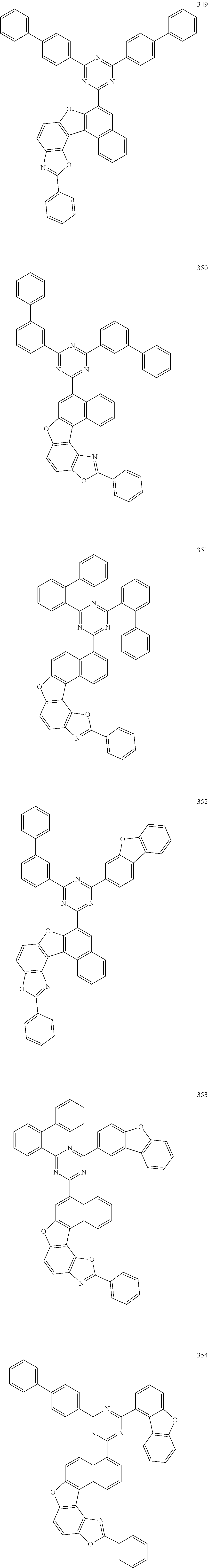

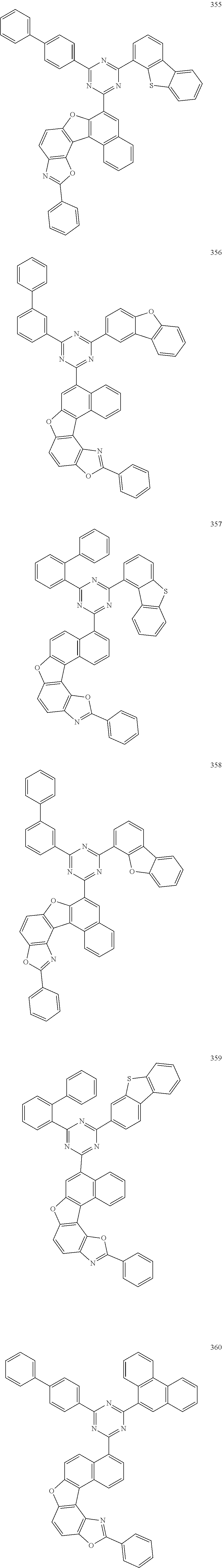

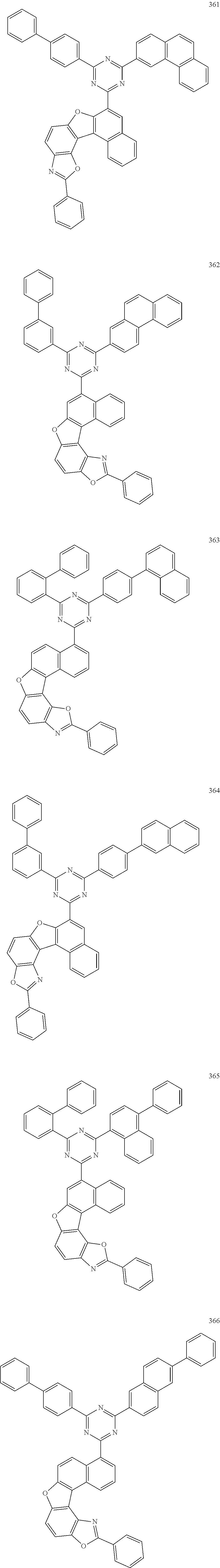

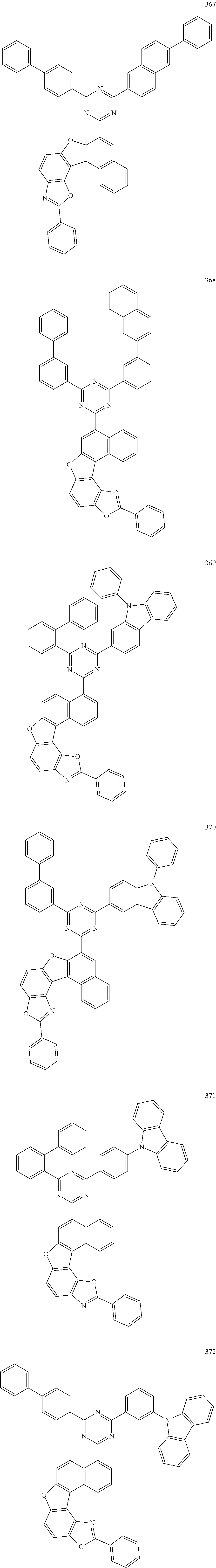

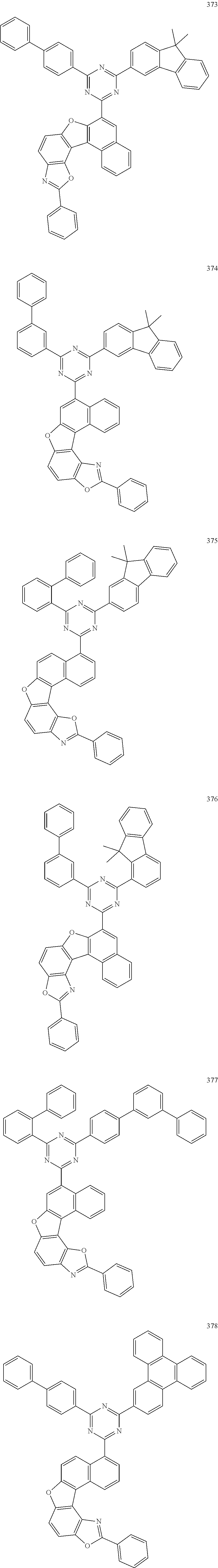

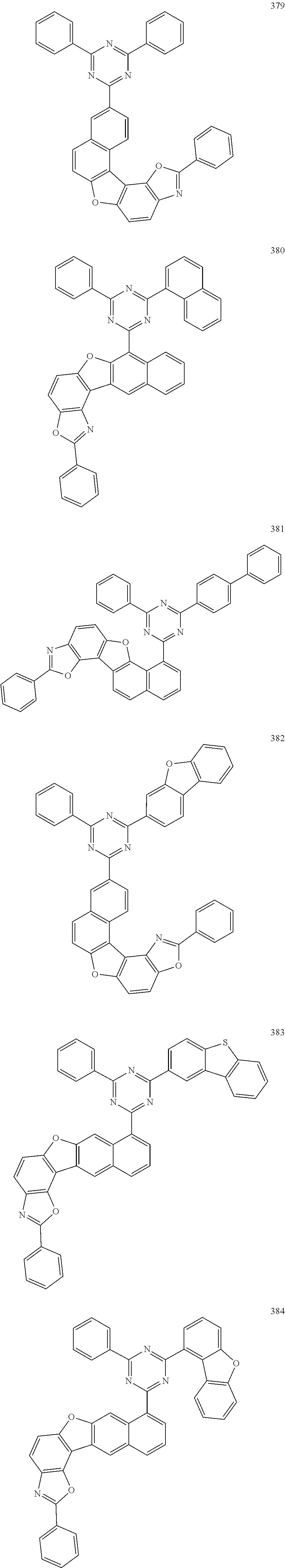

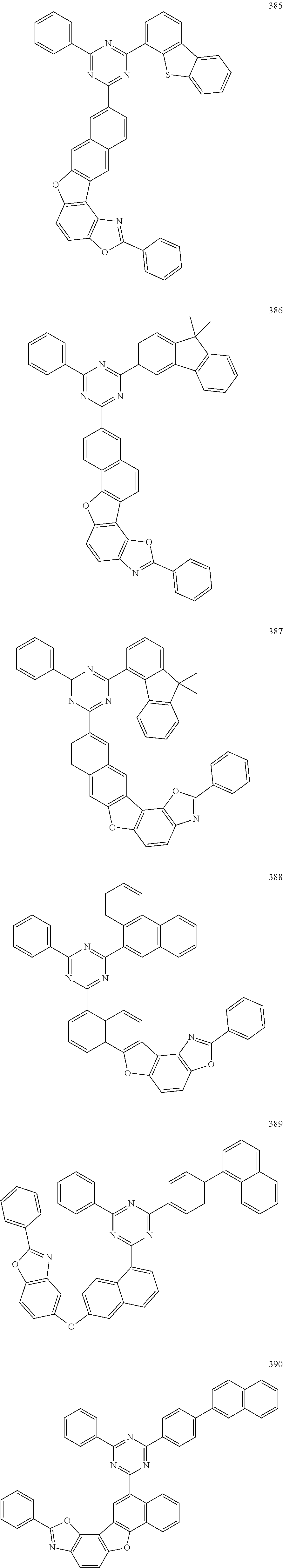

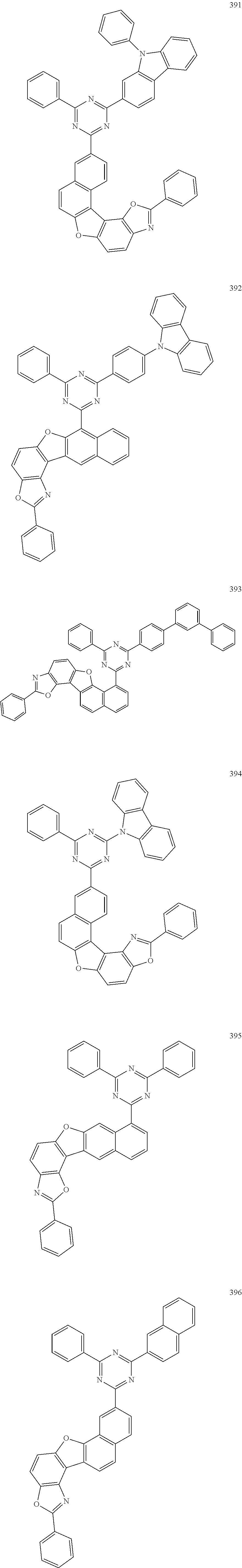

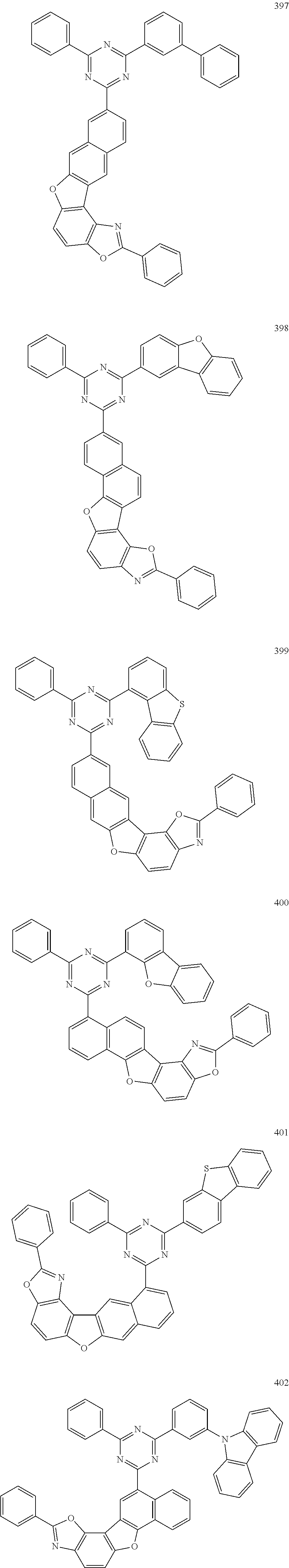

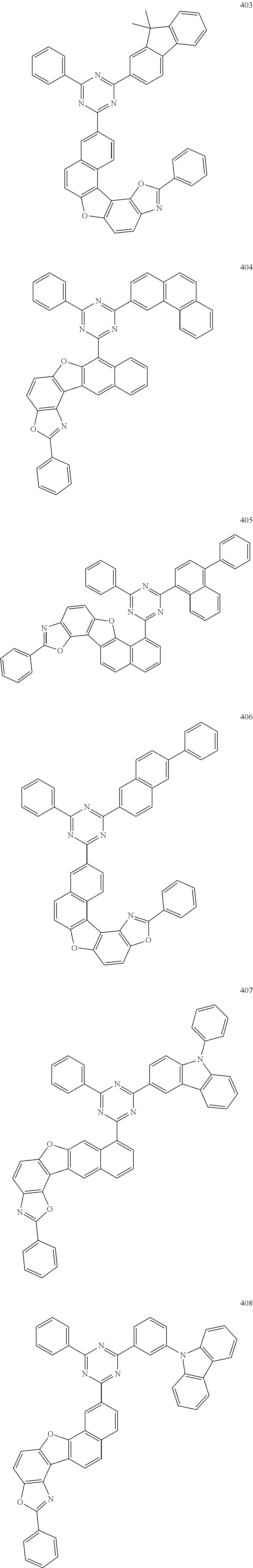

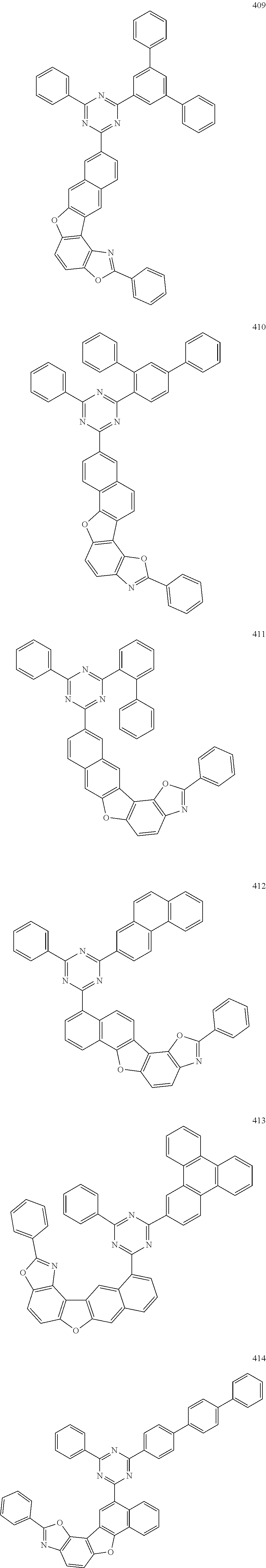

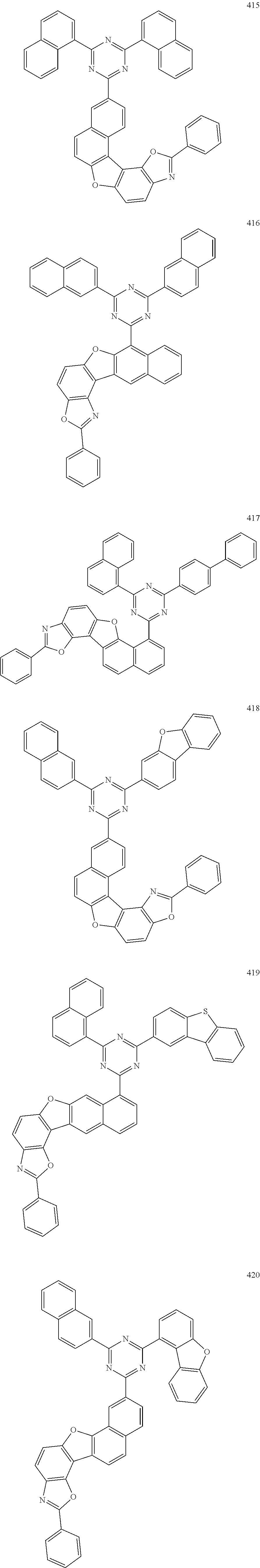

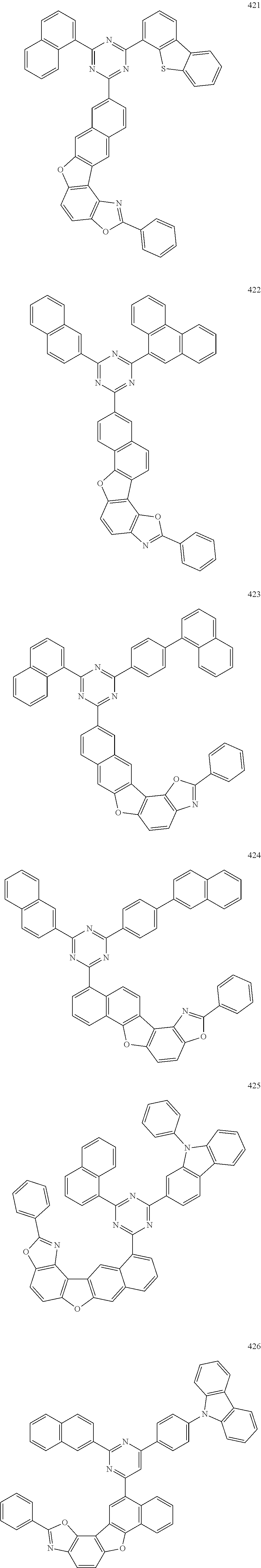

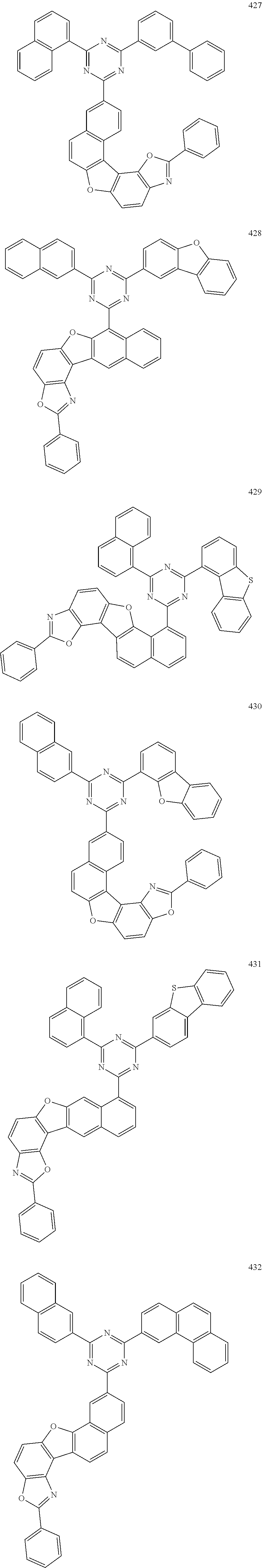

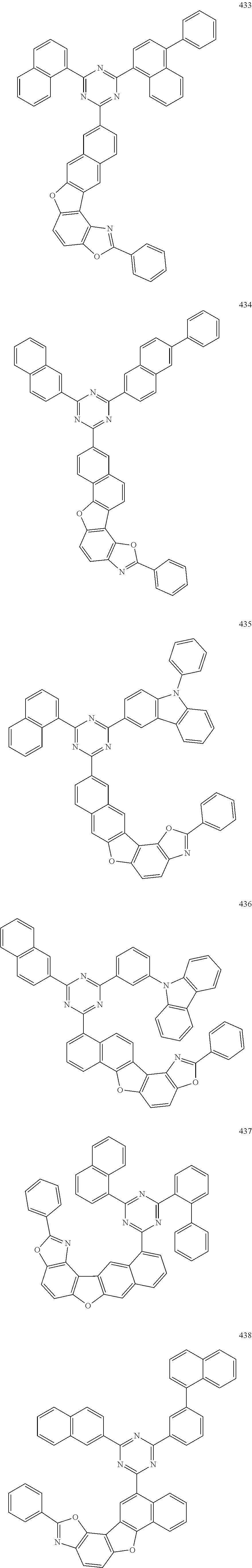

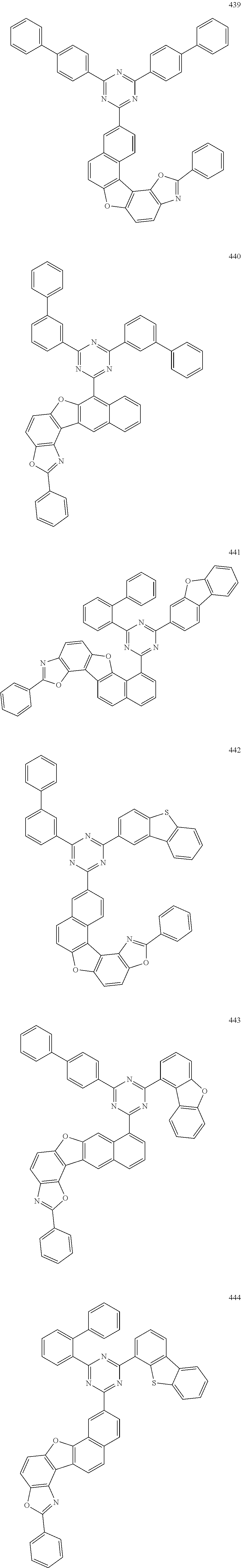

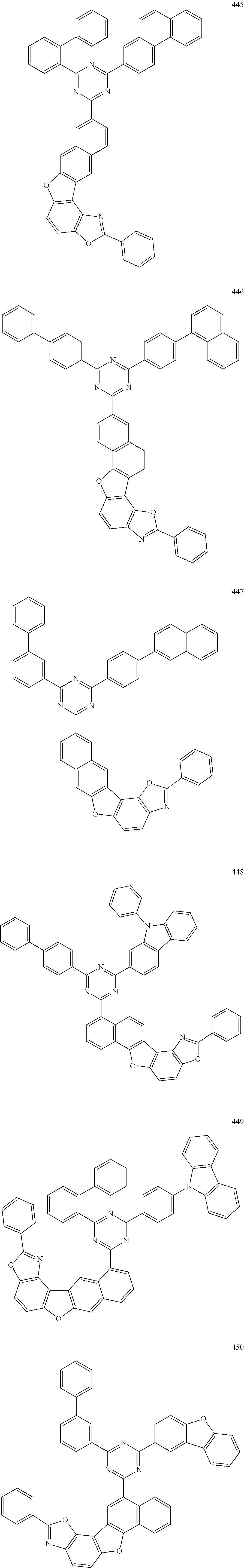

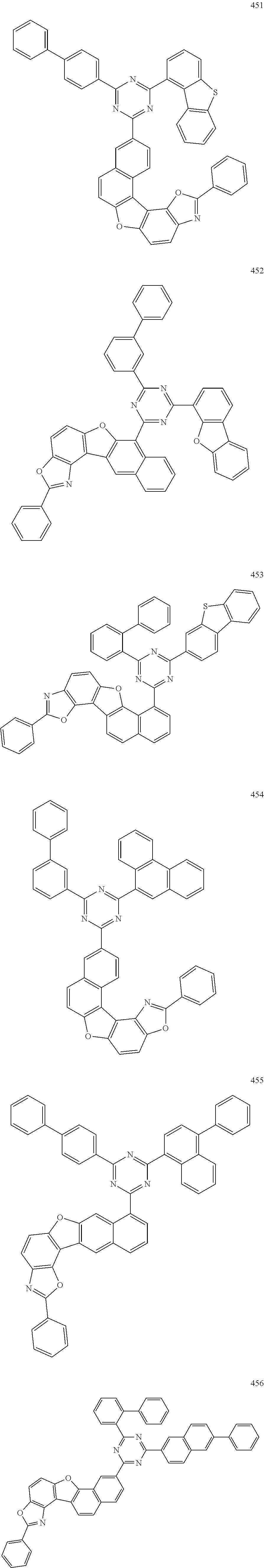

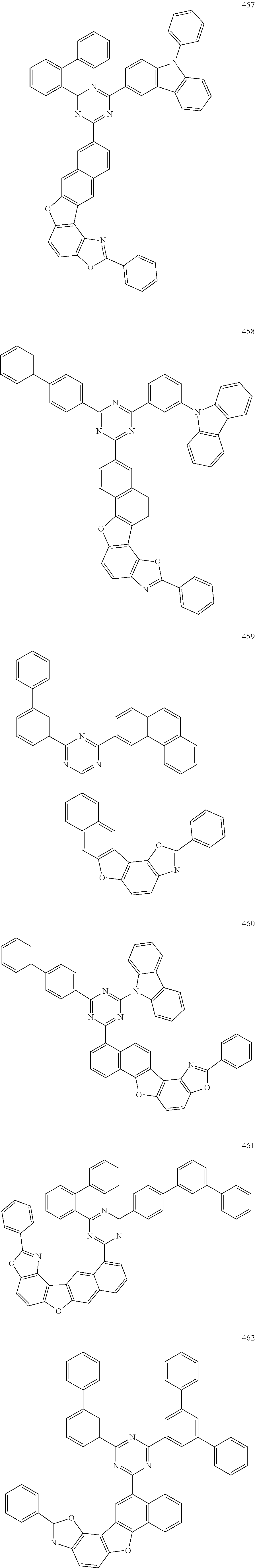

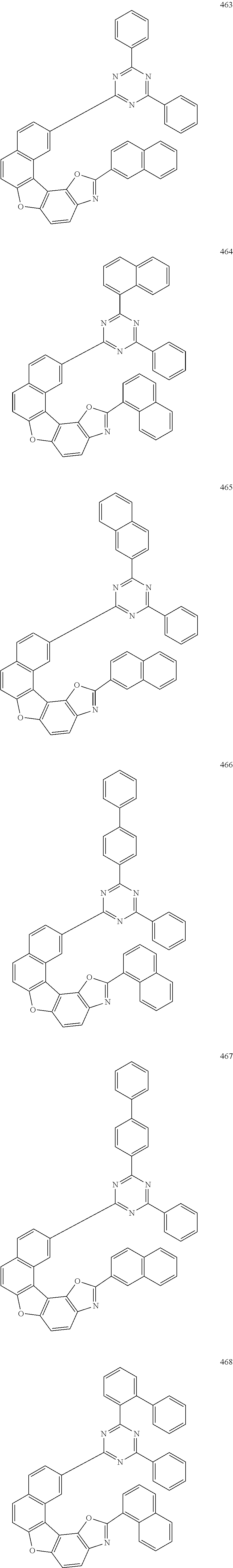

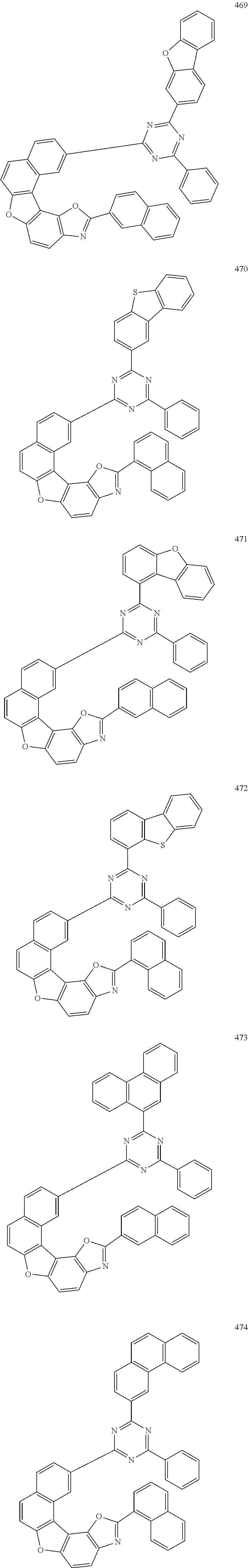

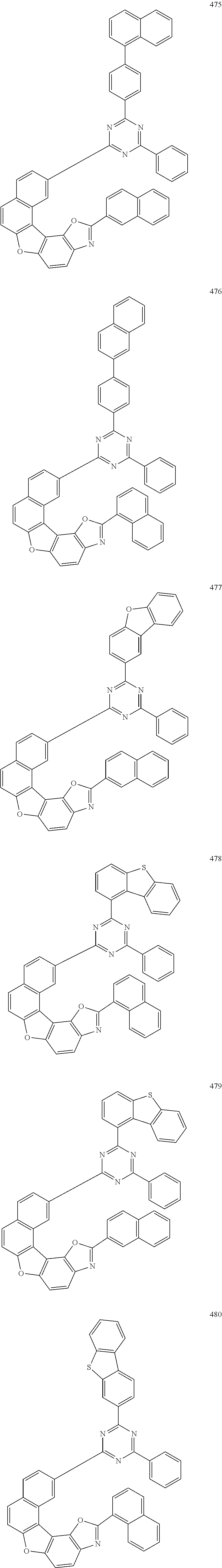

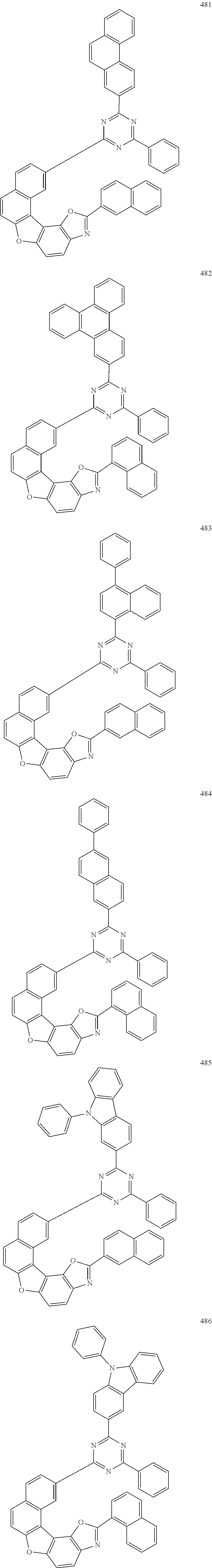

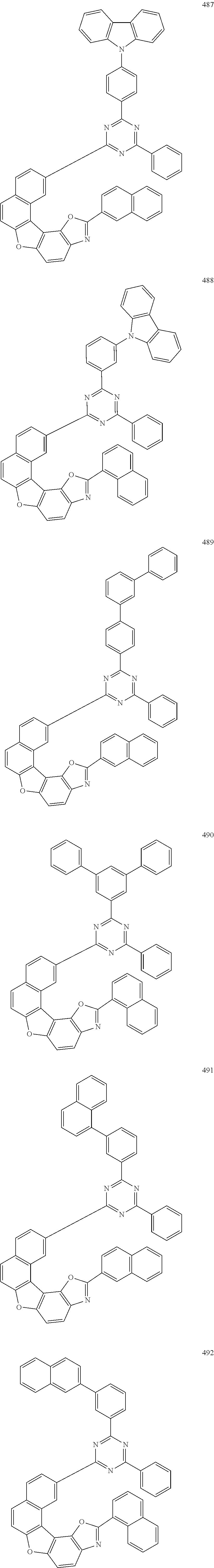

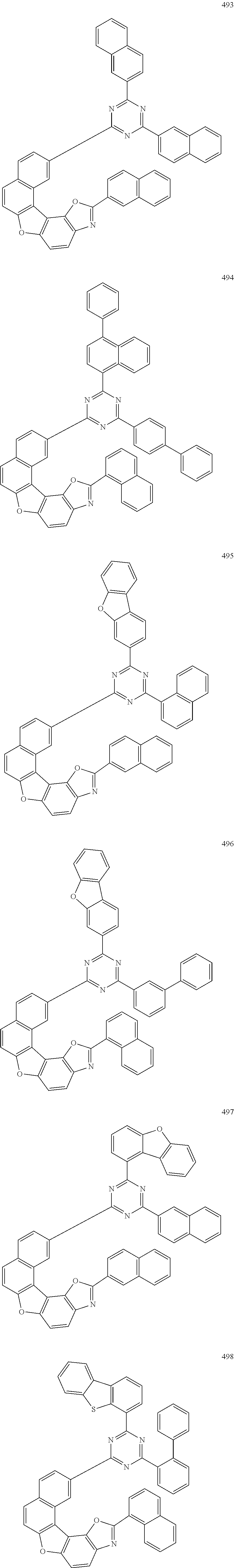

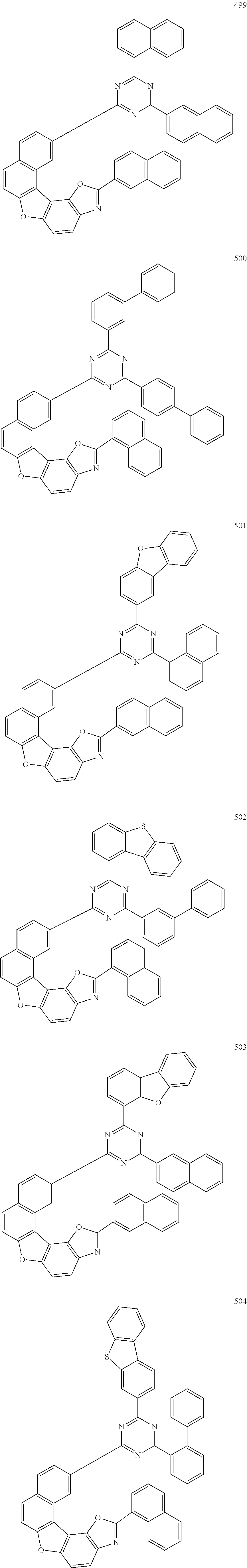

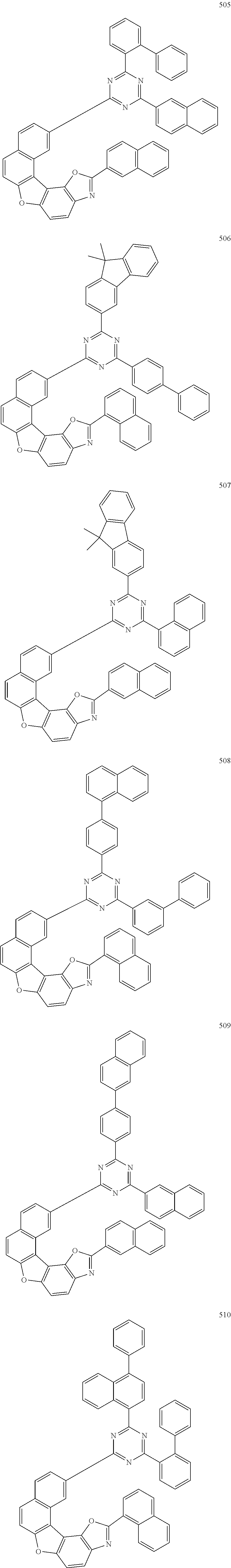

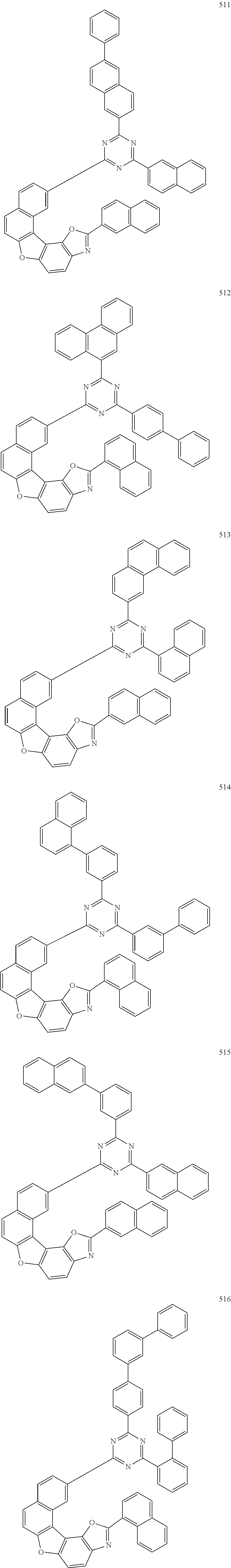

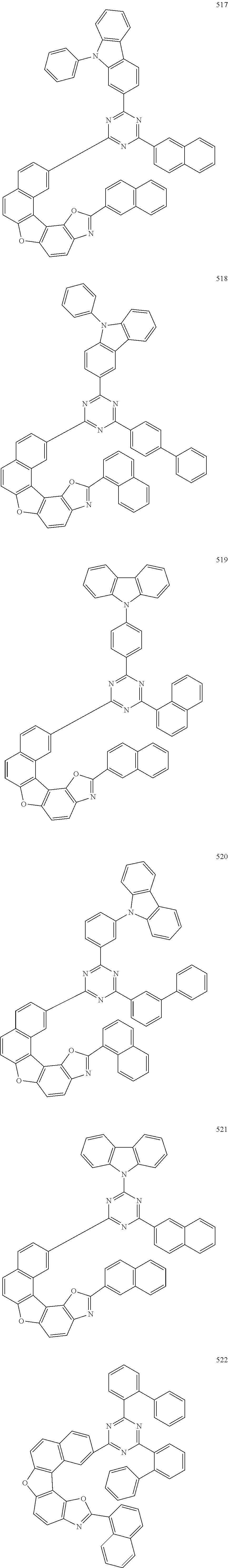

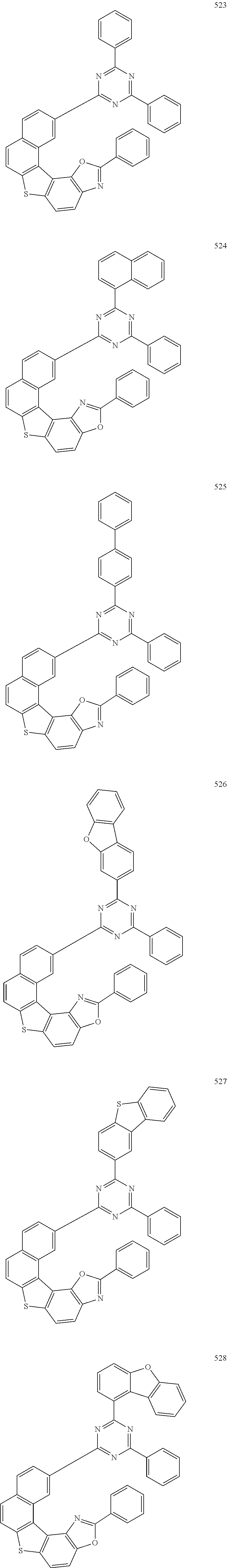

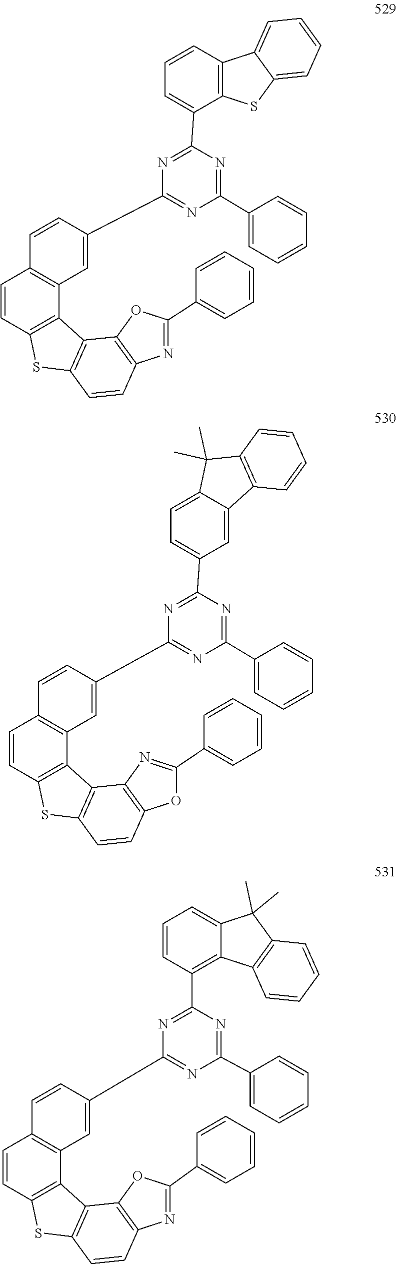 [SMILES: c1ccc(-c2nc(-c3ccc4ccc5sc6ccc7nc(-c8ccccc8)oc7c6c5c4c3)nc(-c3cccc4c3sc3ccccc34)n2)cc1 ; CC1(C)c2ccccc2-c2cc(-c3nc(-c4ccccc4)nc(-c4ccc5ccc6sc7ccc8oc(-c9ccccc9)nc8c7c6c5c4)n3)ccc21 ; CC1(C)c2ccccc2-c2c(-c3nc(-c4ccccc4)nc(-c4ccc5ccc6sc7ccc8nc(-c9ccccc9)oc8c7c6c5c4)n3)cccc21]

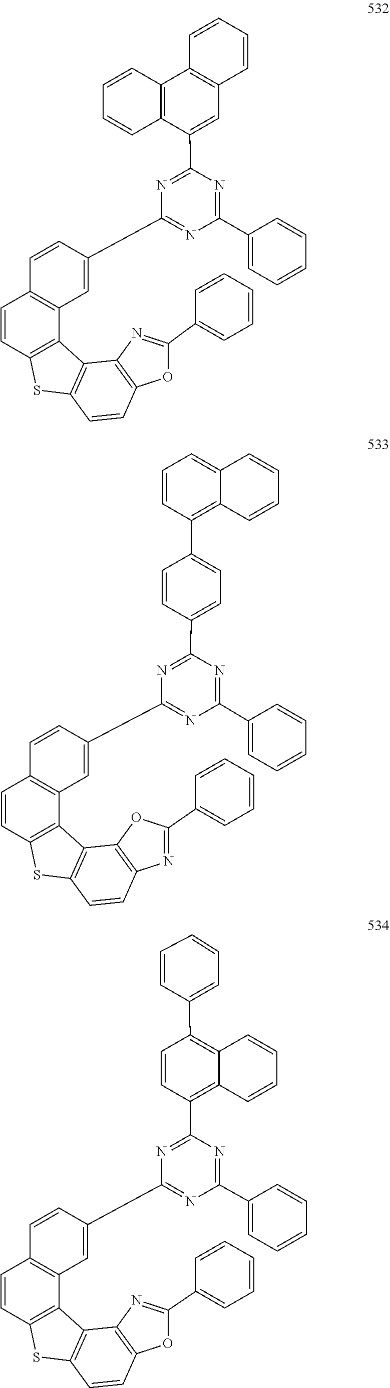 [SMILES: c1ccc(-c2nc(-c3ccc4ccc5sc6ccc7oc(-c8ccccc8)nc7c6c5c4c3)nc(-c3cc4ccccc4c4ccccc34)n2)cc1 ; c1ccc(-c2nc(-c3ccc(-c4cccc5ccccc45)cc3)nc(-c3ccc4ccc5sc6ccc7nc(-c8ccccc8)oc7c6c5c4c3)n2)cc1 ; c1ccc(-c2nc(-c3ccc4ccc5sc6ccc7oc(-c8ccccc8)nc7c6c5c4c3)nc(-c3ccc(-c4ccccc4)c4ccccc34)n2)cc1]

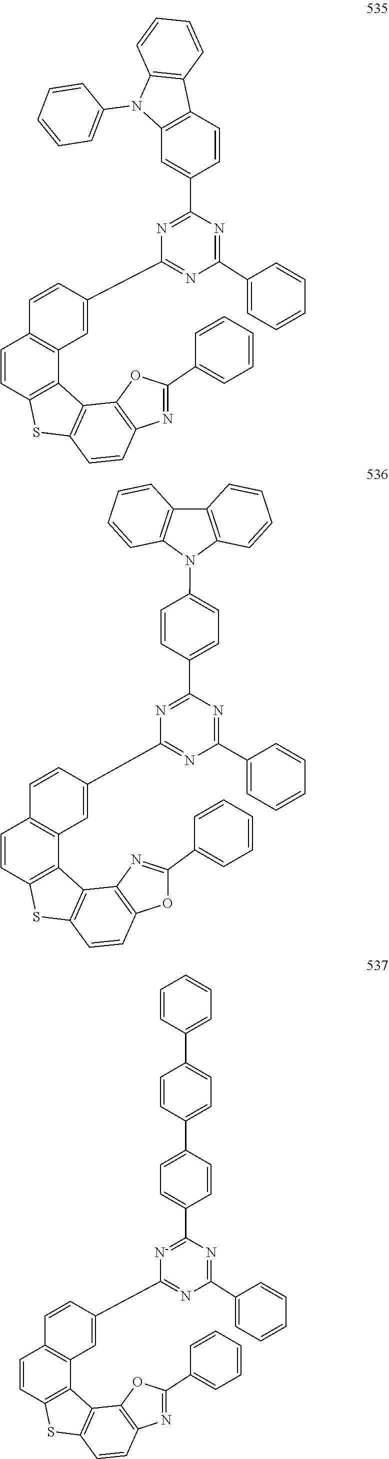

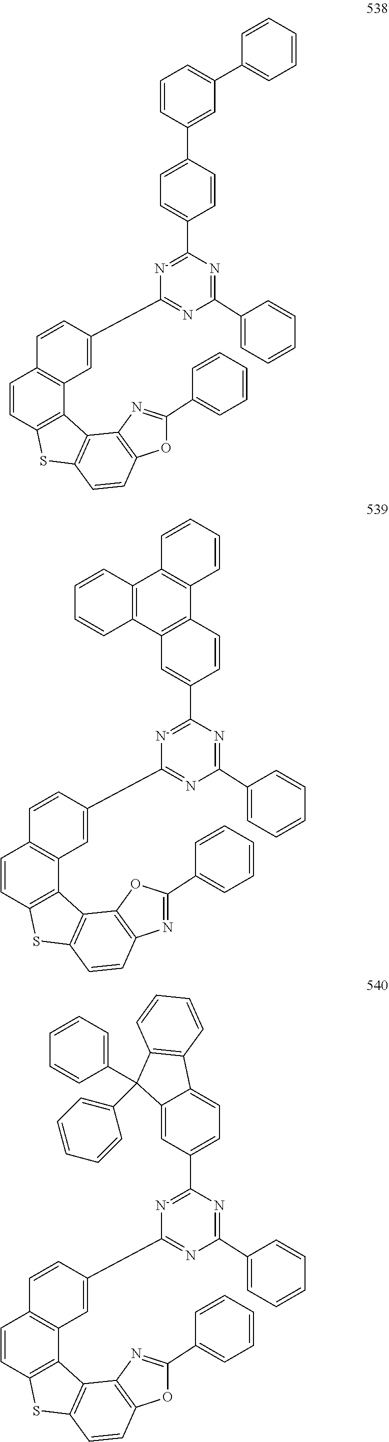

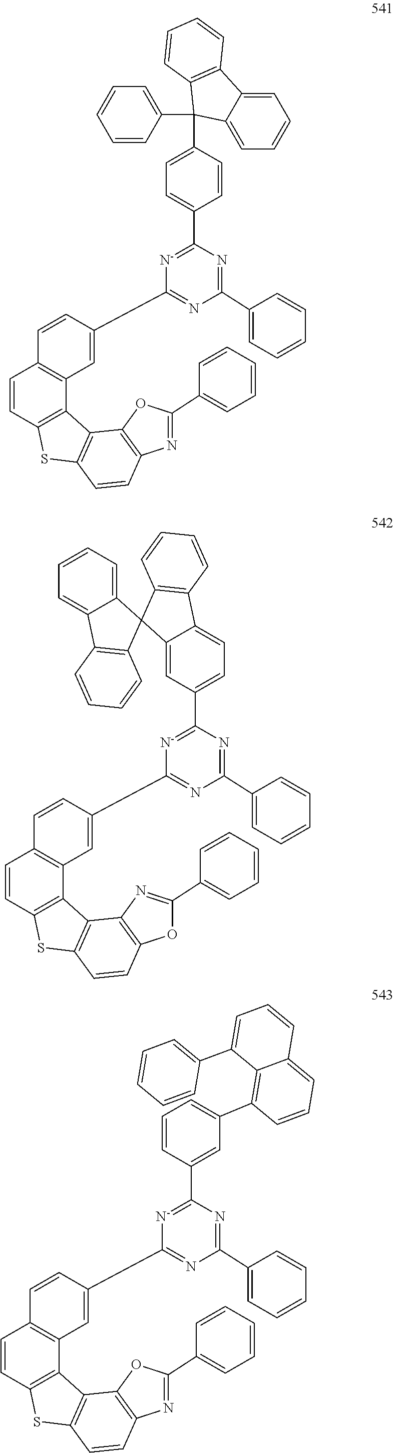

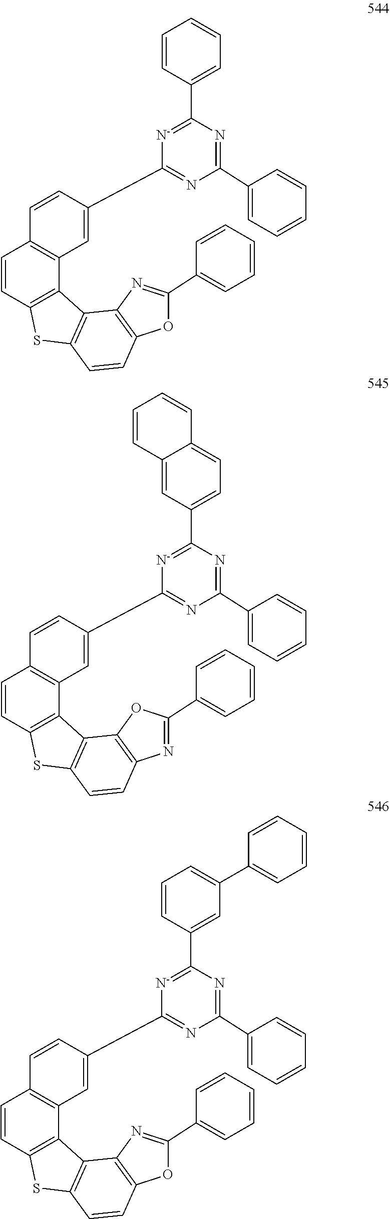

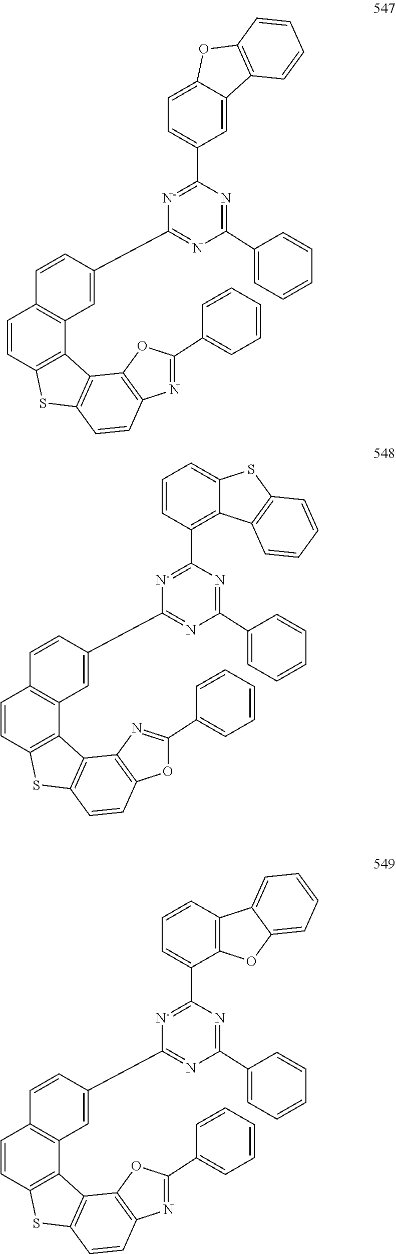

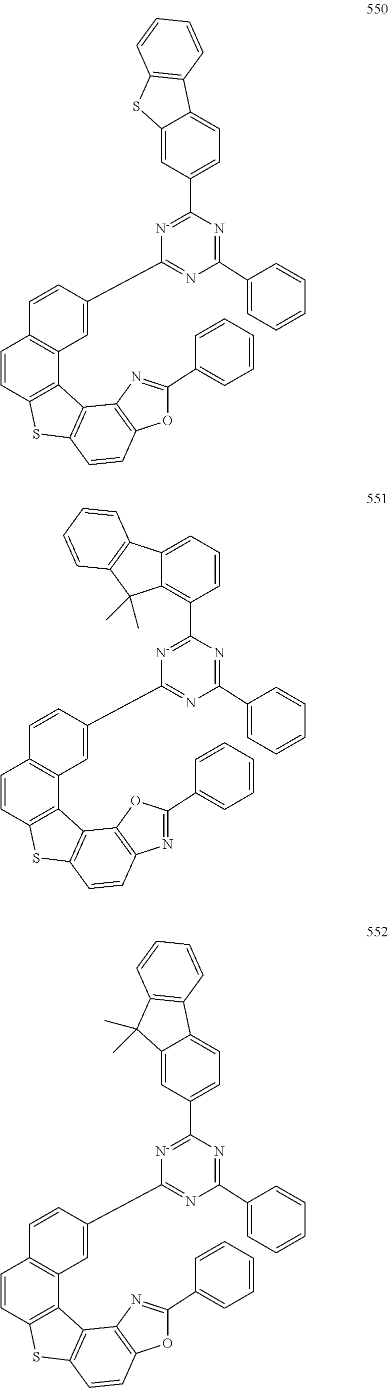

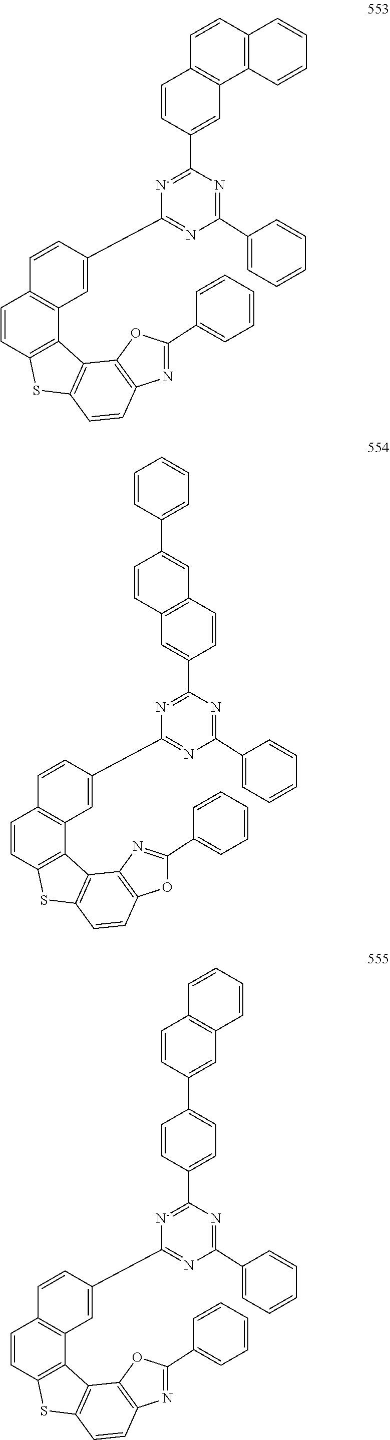

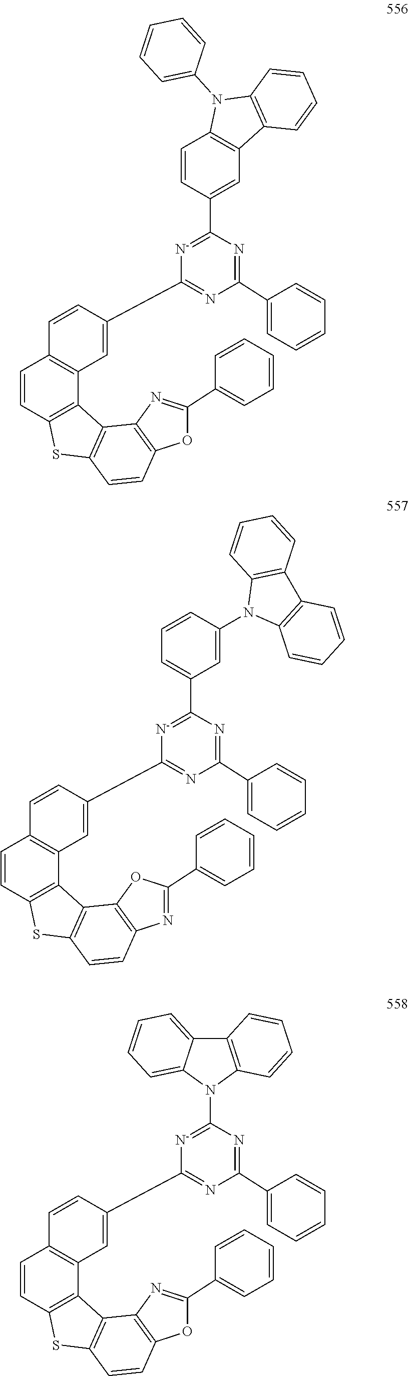

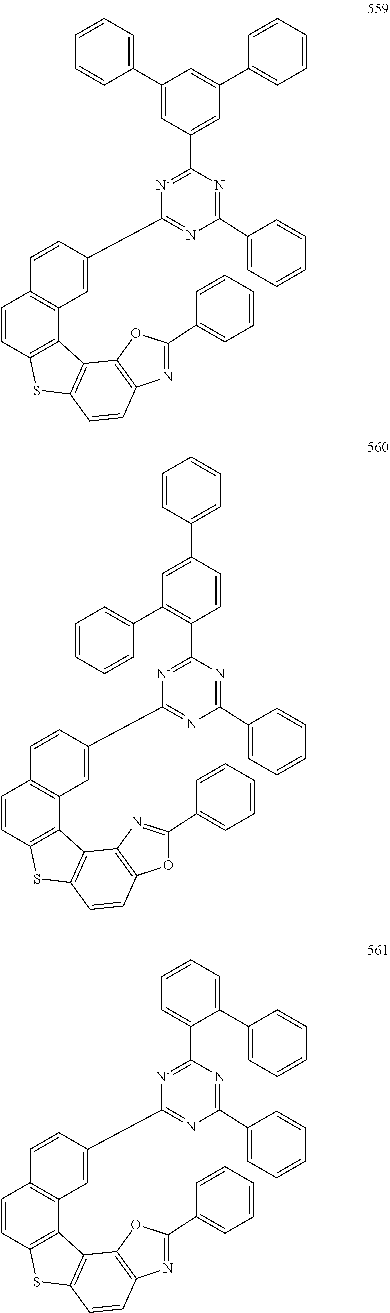

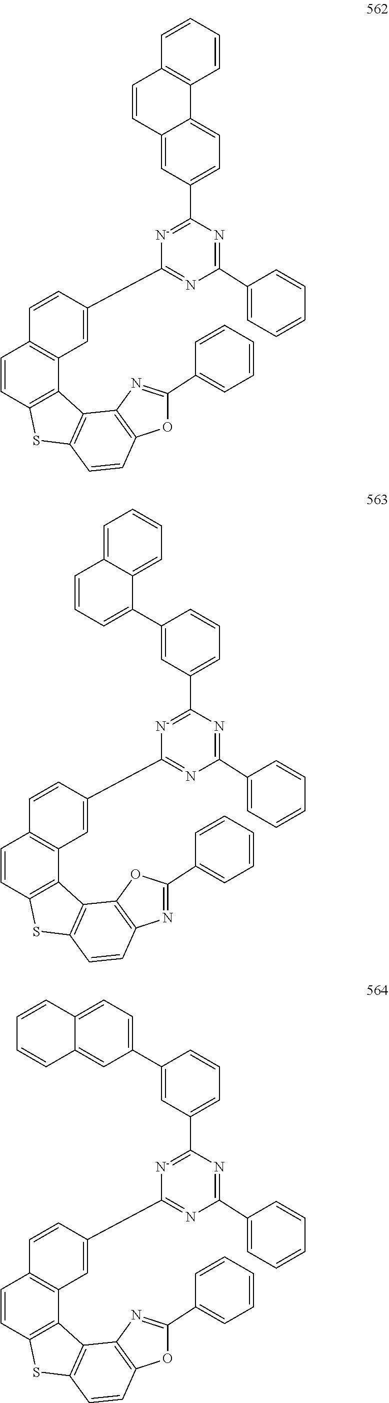

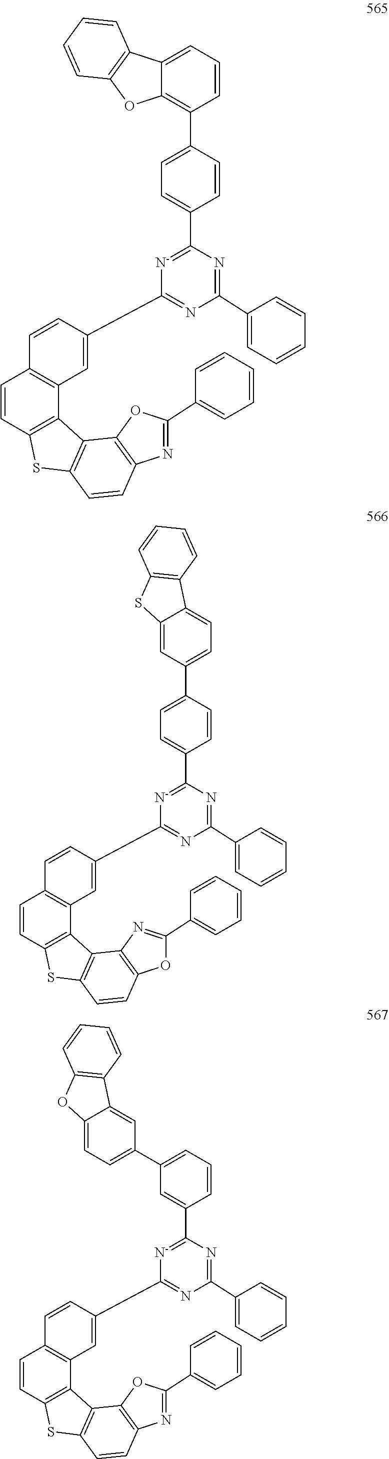

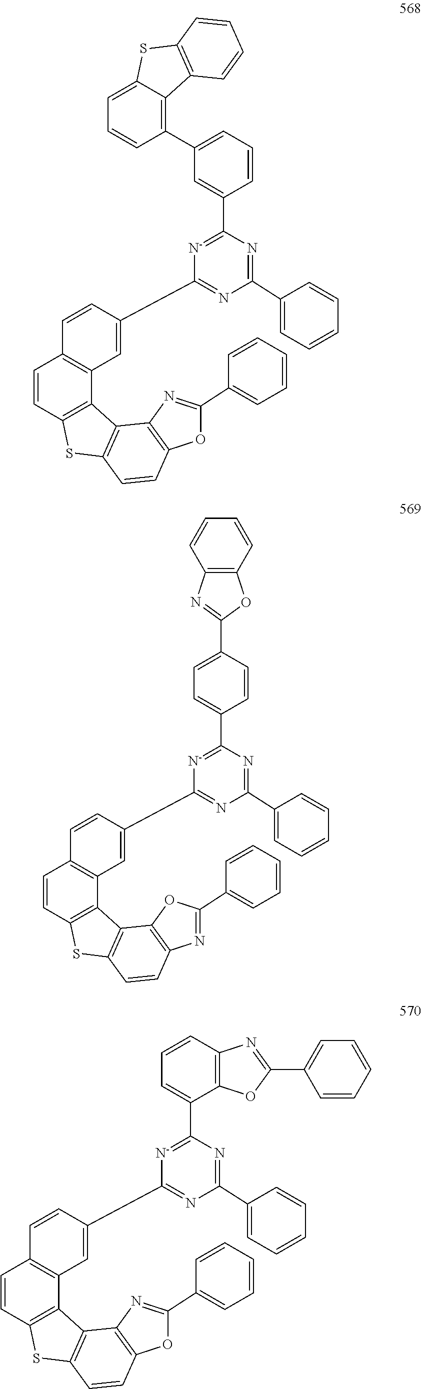

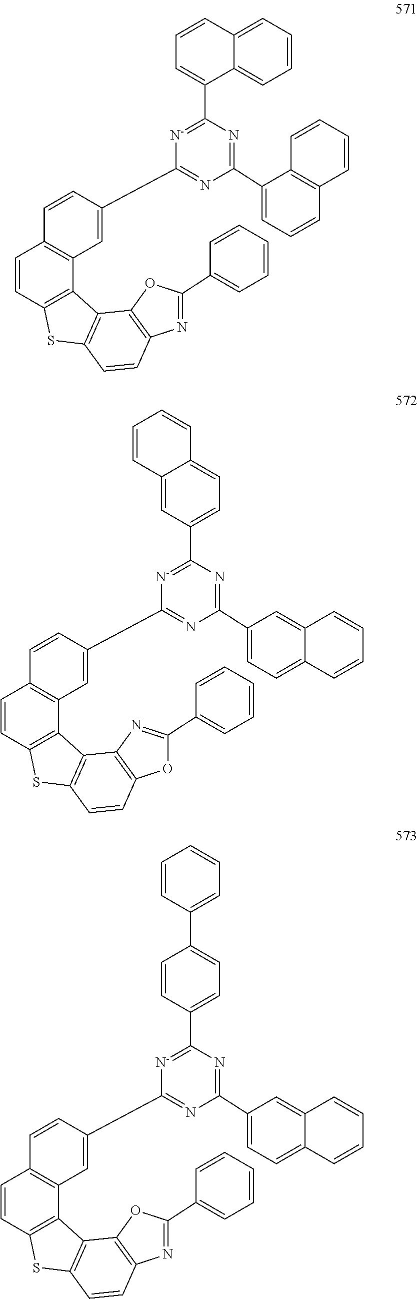

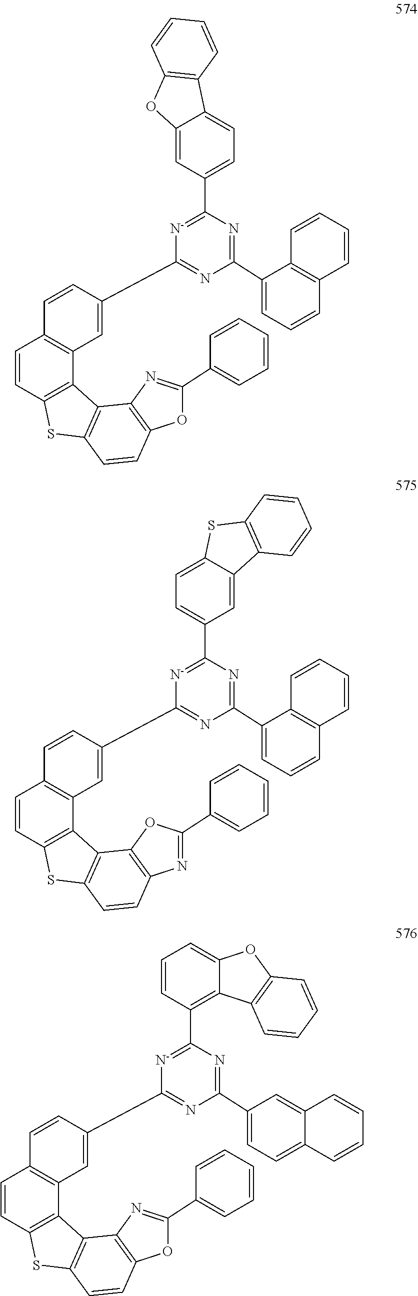

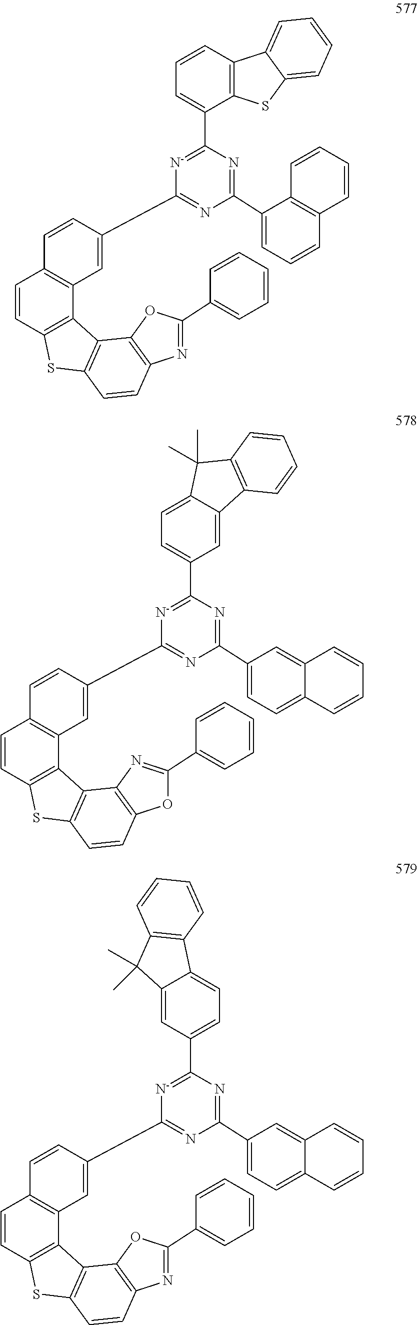

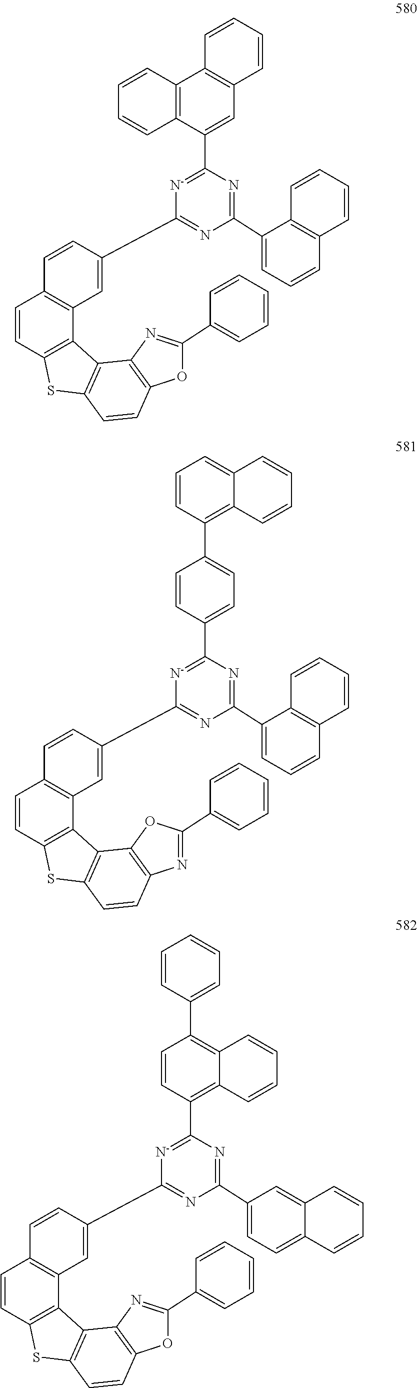

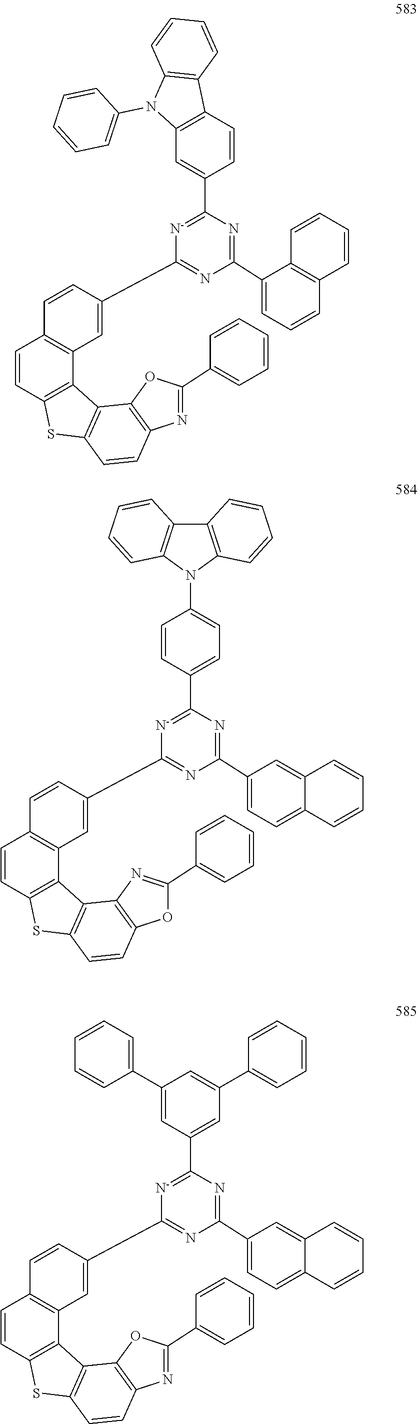

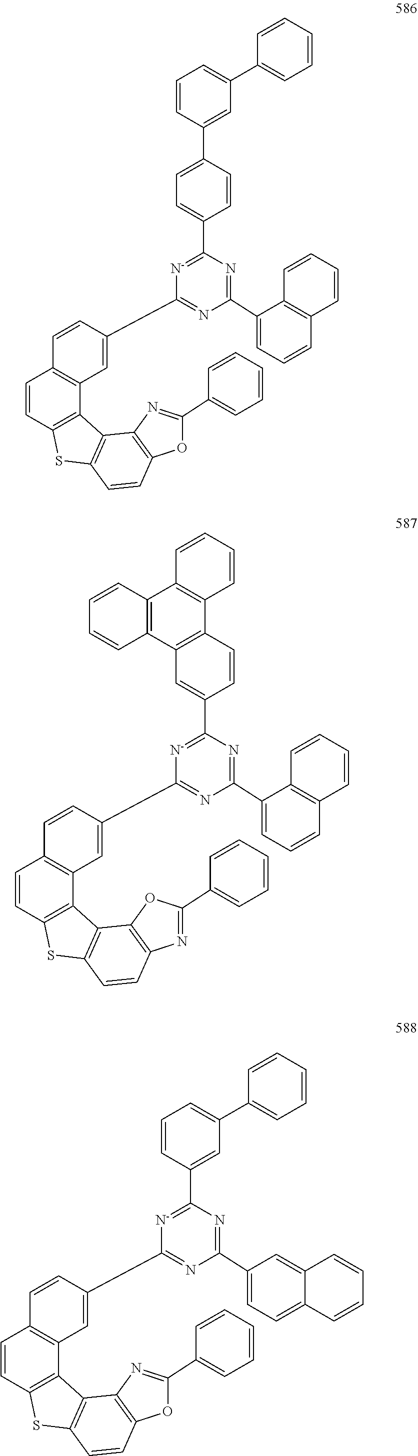

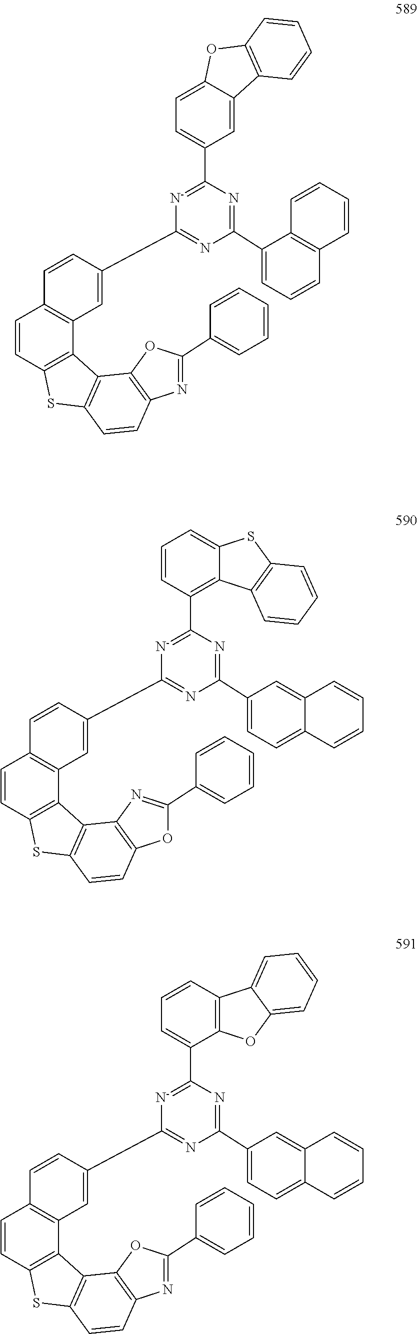

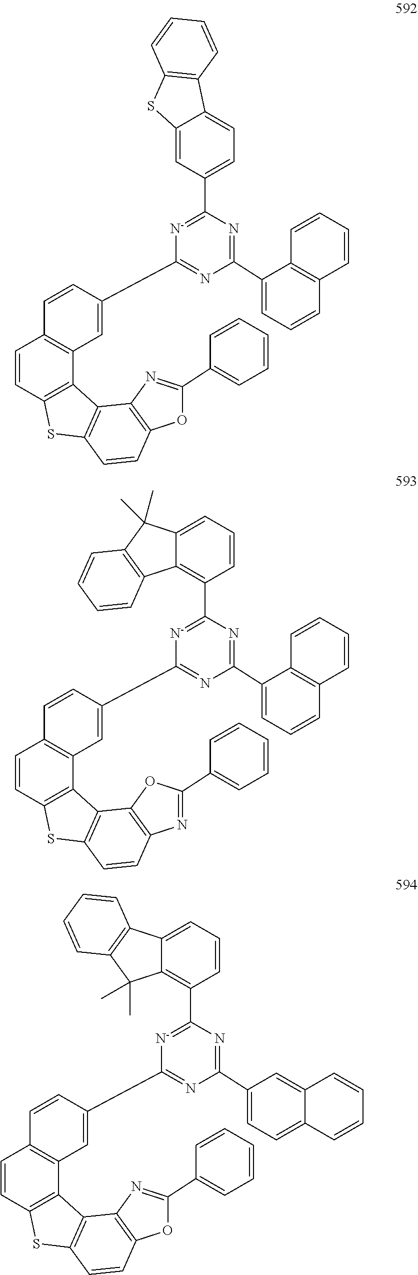

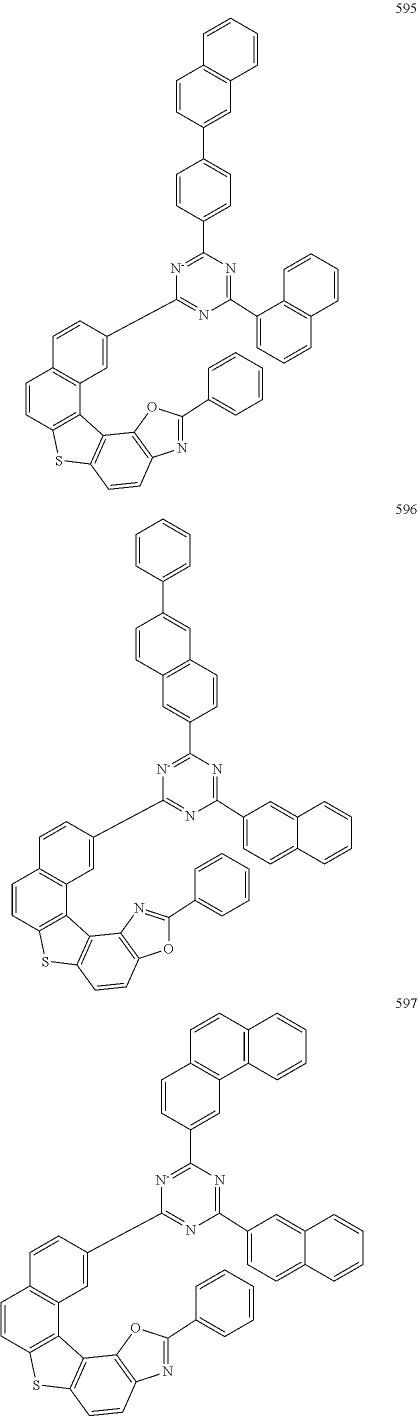

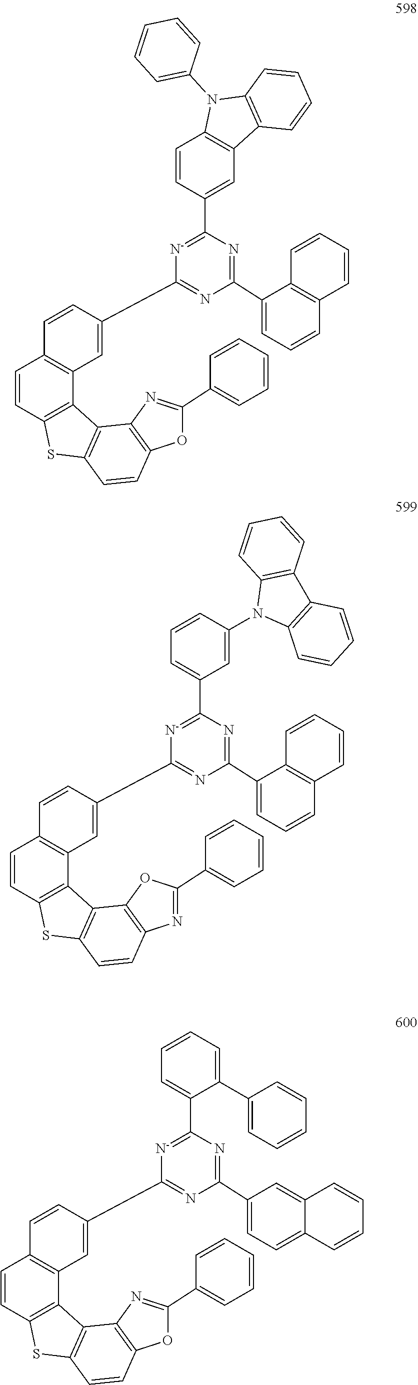

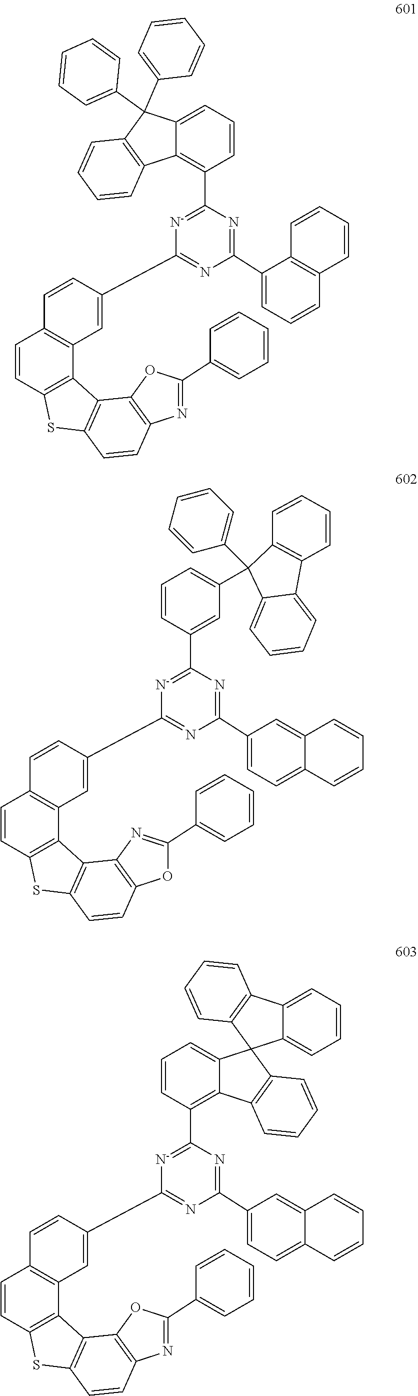

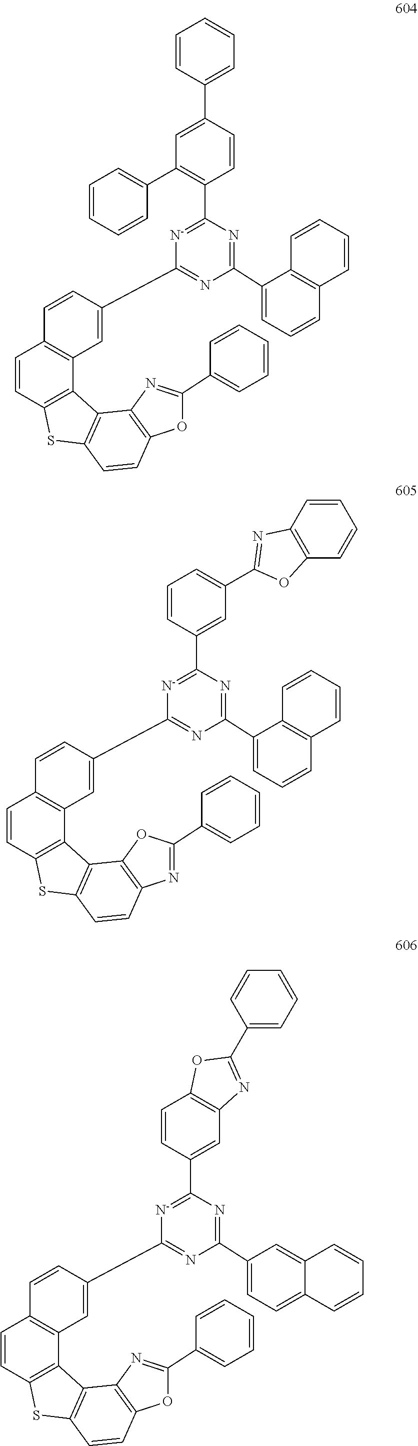

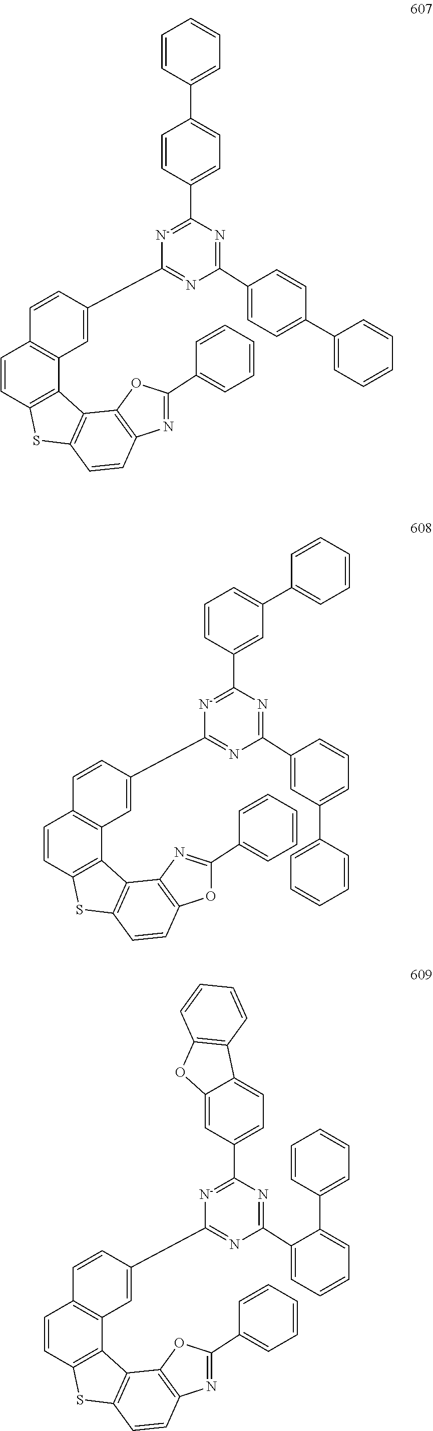

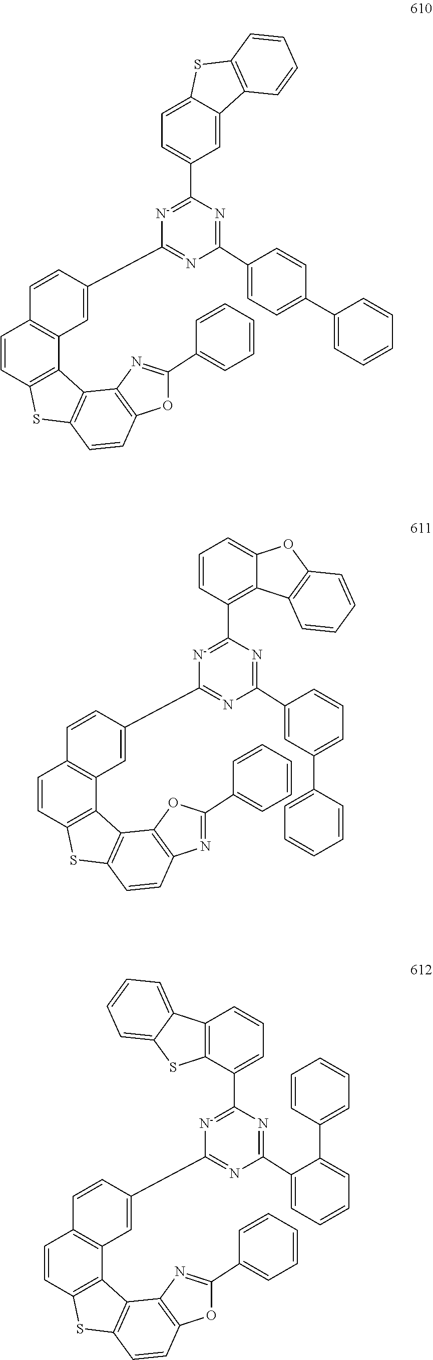

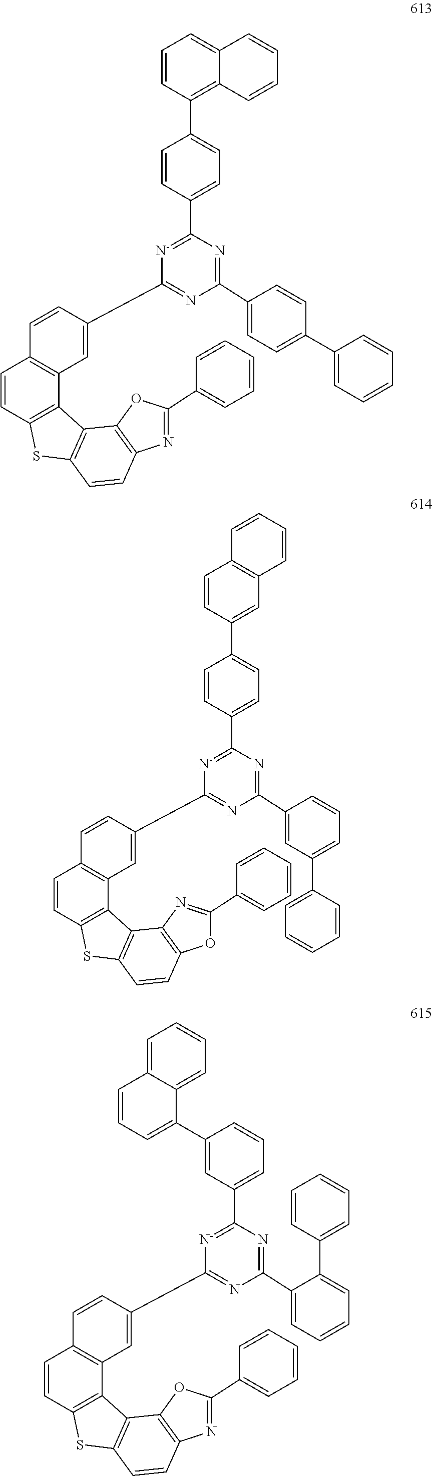

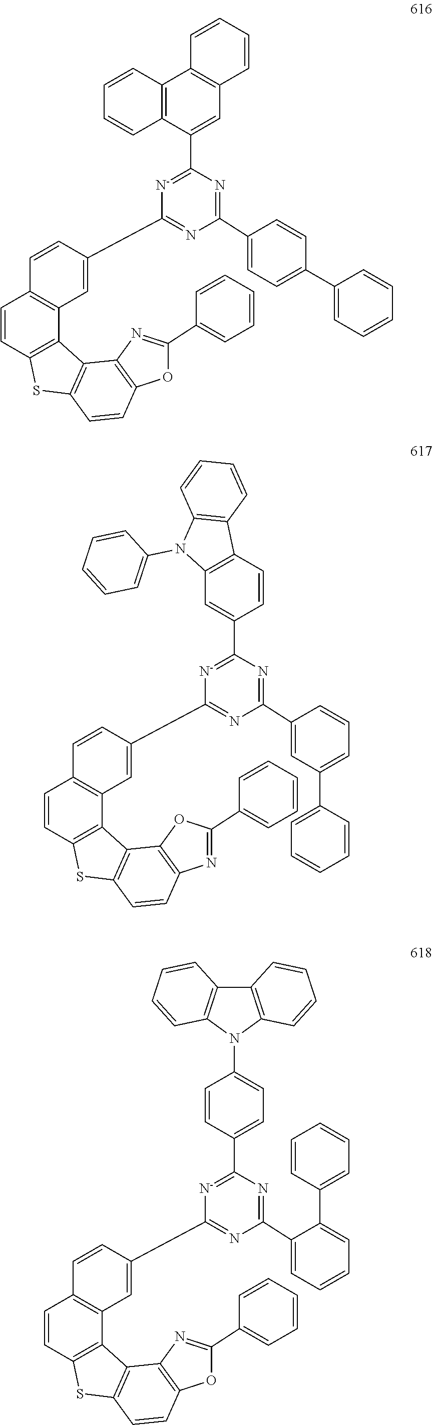

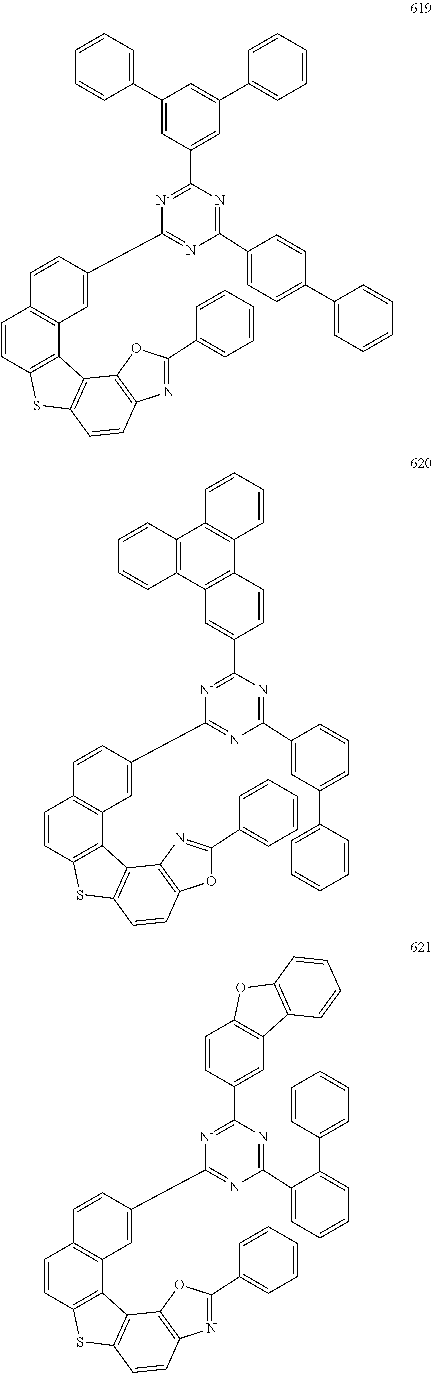

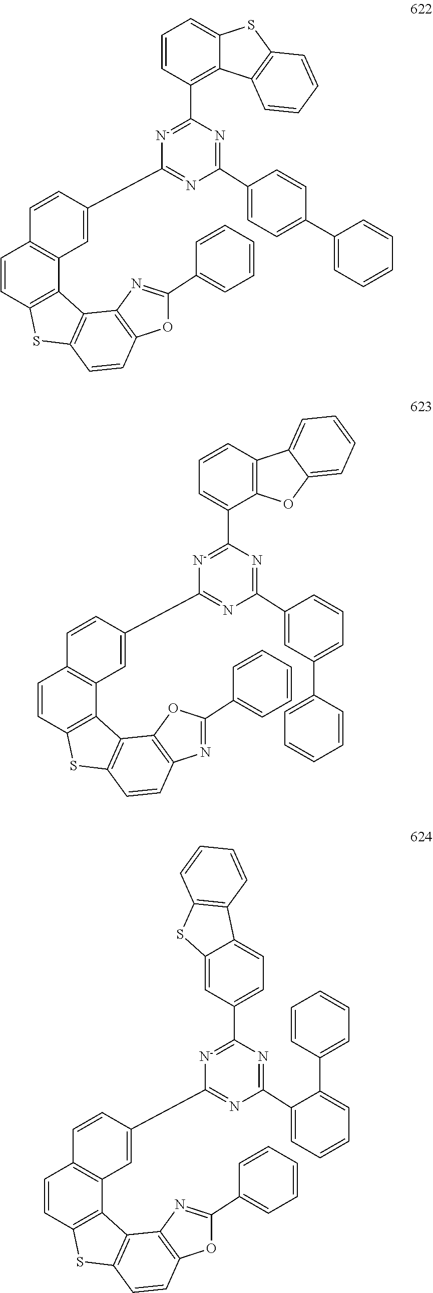

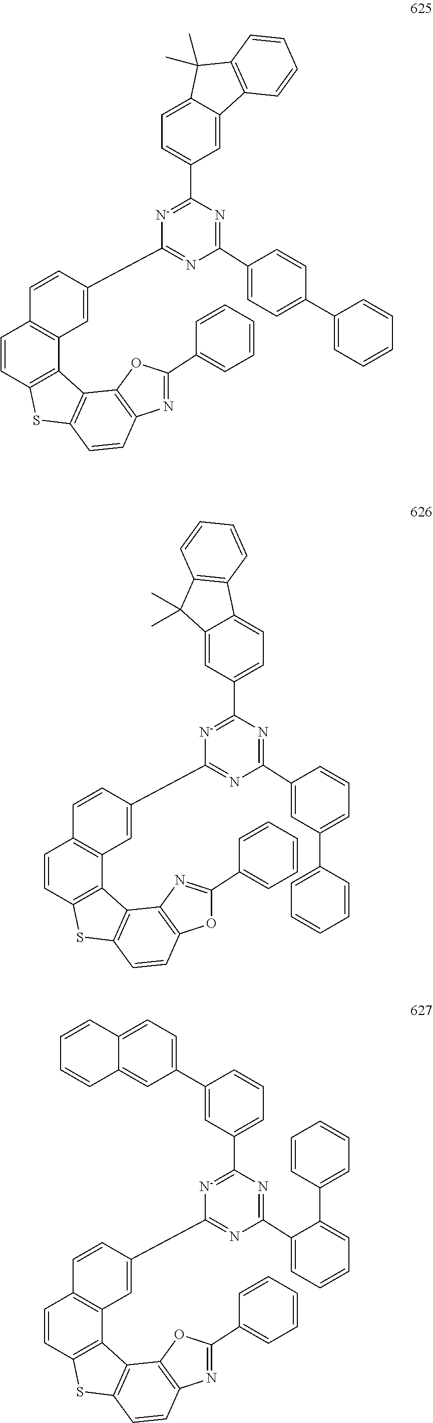

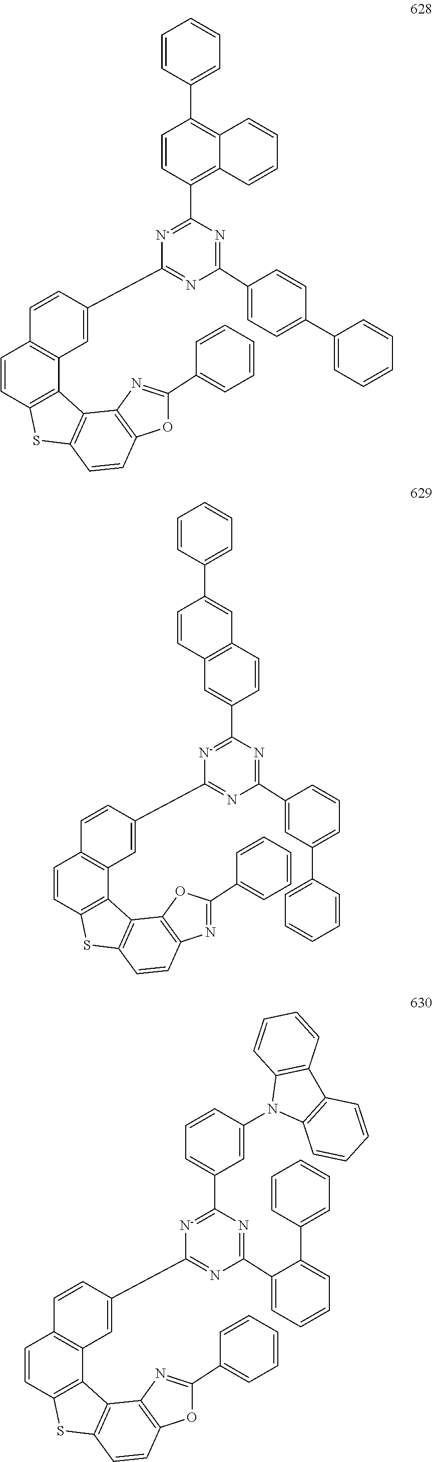

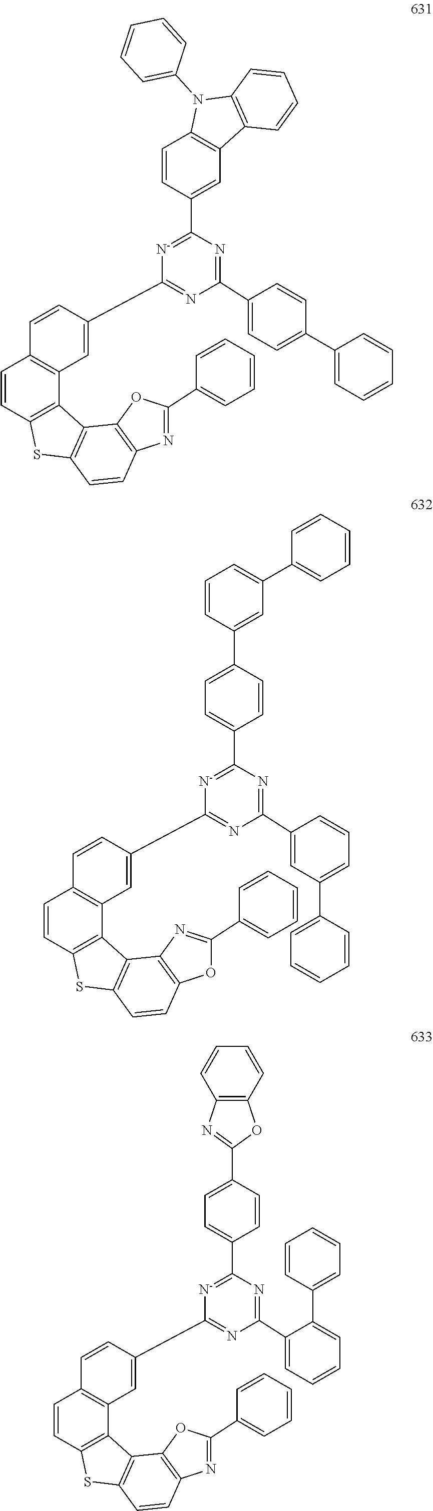

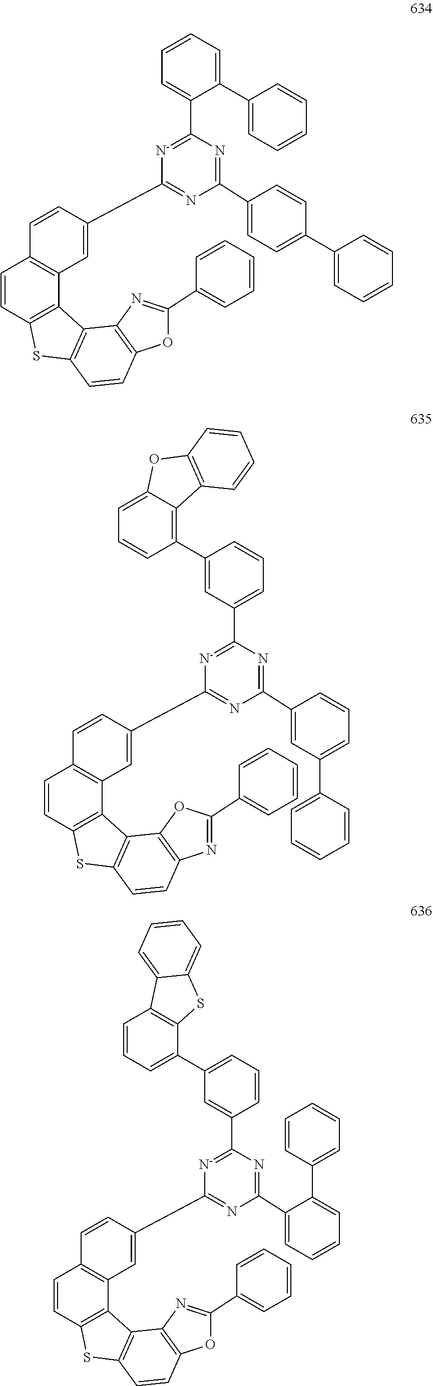

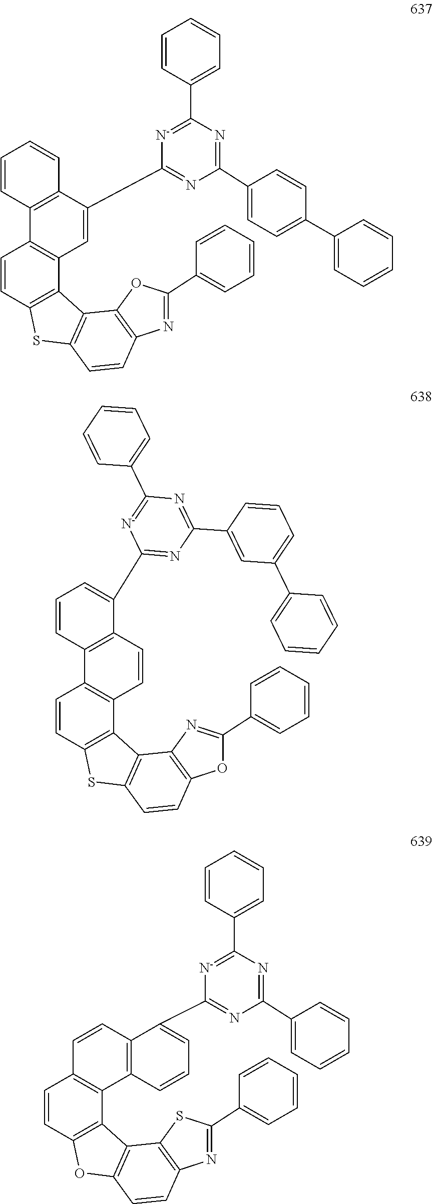

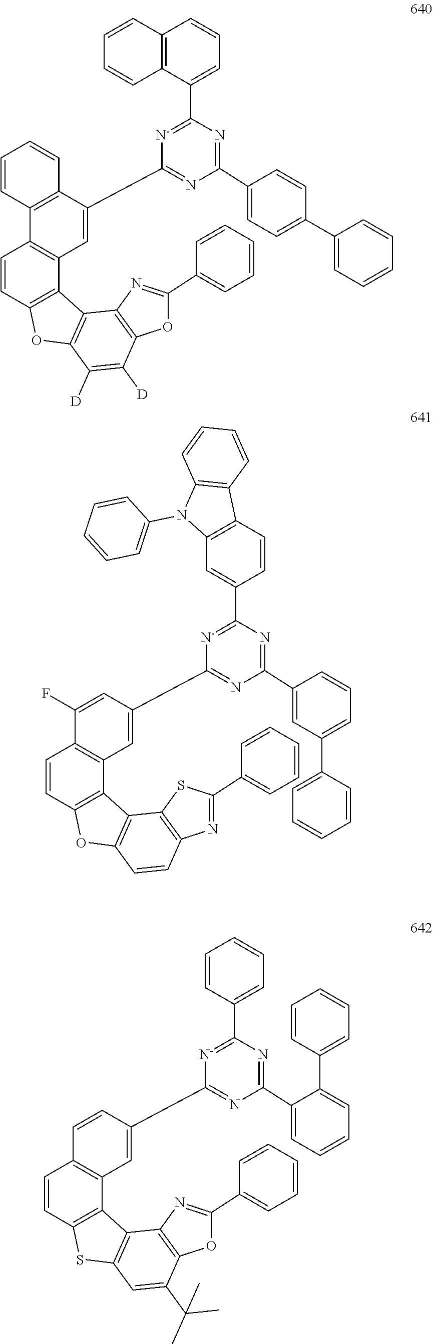

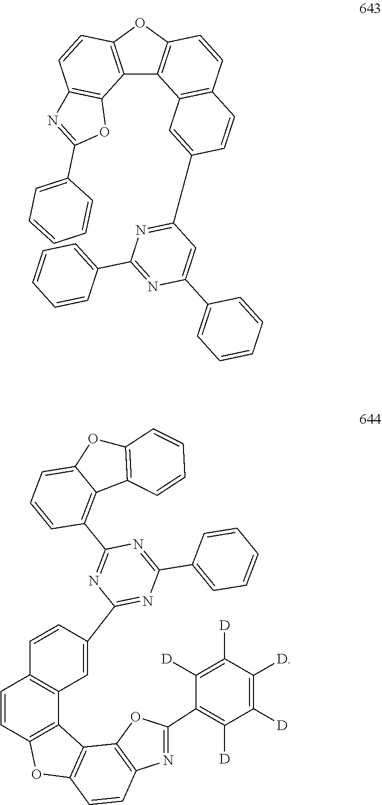

In a second aspect, the present application provides an organic electroluminescent device comprising an anode, a cathode, and a functional layer disposed between the anode and the cathode. The functional layer comprises the nitrogen-containing compound described in the first aspect of the present application.

The nitrogen-containing compound provided in the present application may be used to form at least one organic film layer in the functional layer so as to improve properties such as luminescence efficiency and service life of the organic electroluminescent device.

Optionally, the functional layer comprises an organic light emitting layer comprising the nitrogen-containing compound. The organic light emitting layer may be composed of the nitrogen-containing compound provided in the present application, or may be composed of the nitrogen-containing compound provided in the present application together with other materials.

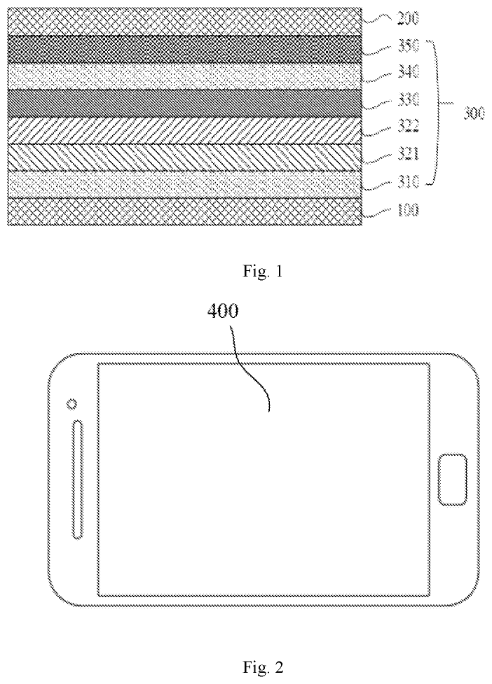

According to a specific embodiment, the organic electroluminescent device is as shown in FIG. 1. The organic electroluminescent device may comprise an anode 100, a hole injection layer 310, a first hole transport layer 321, a second hole transport layer (hole auxiliary layer) 322, an organic light emitting layer 330, an electron transport layer 340, an electron injection layer 350, and a cathode 200 that are stacked in sequence.

In the present application, the anode 100 comprises an anode material, which is preferably a large-work function material contributing to injection of holes into the functional layer. Specific examples of the anode material include, but are not limited to: metals such as nickel, platinum, vanadium, chromium, copper, zinc, gold, and alloys thereof, metal oxides such as zinc oxide, indium oxide, indium tin oxide (ITO), and indium zinc oxide (IZO); combinations of metals and oxides, such as ZnO:Al or SnO₂:Sb; and conductive polymers such as poly(3-methylthiophene), poly[3,4-(ethylene-1,2-dioxy)thiophene](PEDT), polypyrrole, and polyaniline. Preferably, a transparent electrode comprising indium tin oxide (ITO) is included as the anode.

In the present application, the hole transport layer may comprise one or more hole transport materials. The transport materials may be selected from carbazole polymers, carbazole-linked triarylamine compounds, and other types of compounds, and may be specifically selected from the following compounds or any combinations thereof:

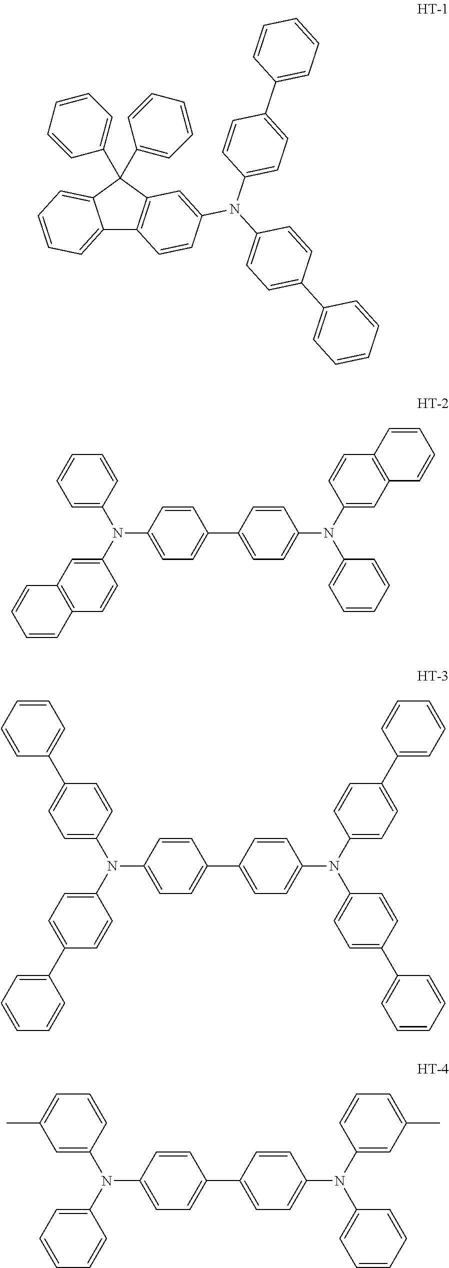

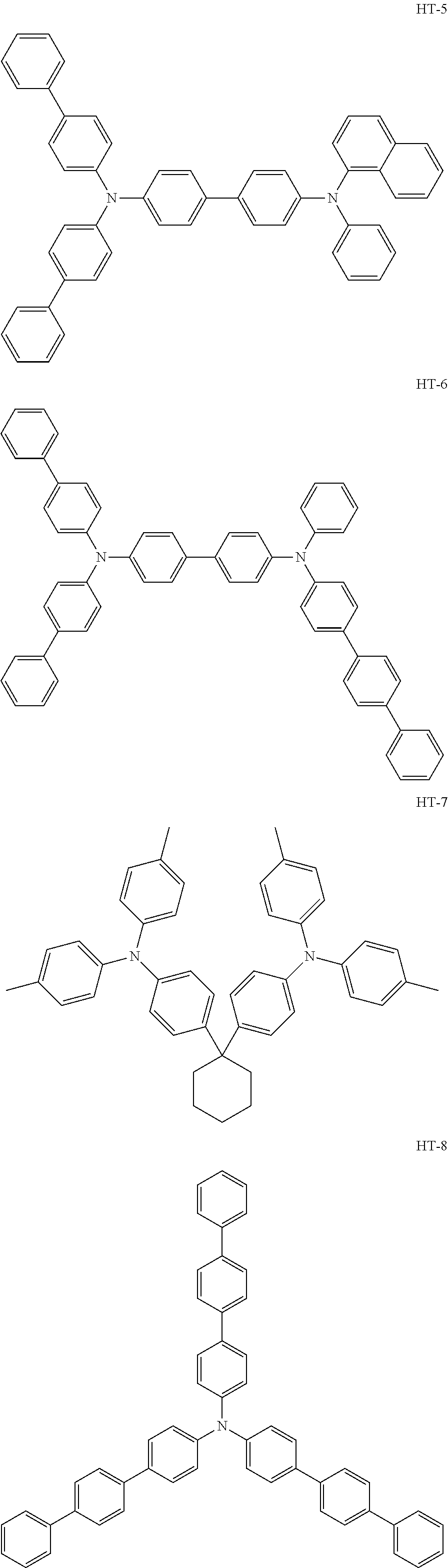

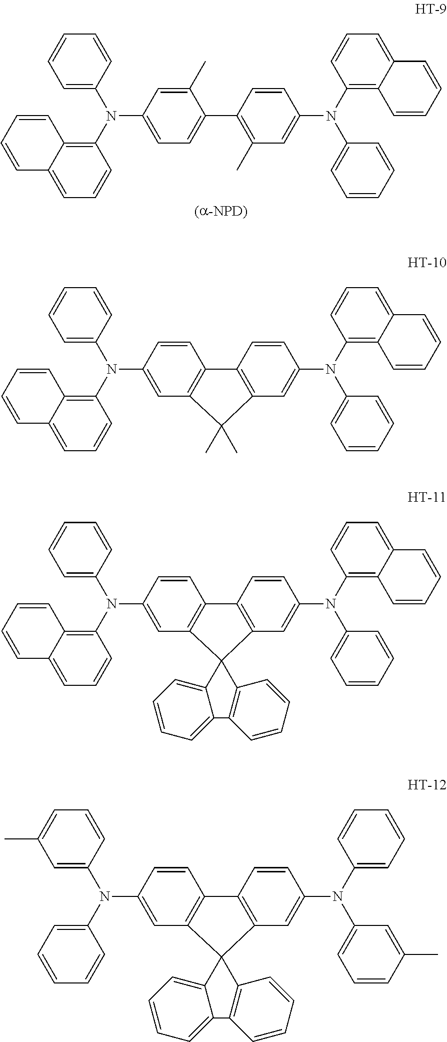

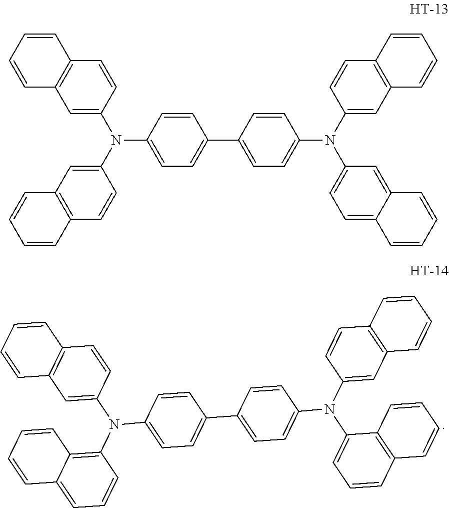 [SMILES: c1ccc2cc(N(c3ccc(-c4ccc(N(c5ccc6ccccc6c5)c5ccc6ccccc6c5)cc4)cc3)c3ccc4ccccc4c3)ccc2c1 ; c1ccc2cc(N(c3ccc(-c4ccc(N(c5ccc6ccccc6c5)c5cccc6ccccc56)cc4)cc3)c3cccc4ccccc34)ccc2c1]

In an embodiment, the first hole transport layer 321 is composed of α-NPD.

In an embodiment, the second hole transport layer 322 is composed of HT-1.

Optionally, a hole injection layer 310 is further provided between the anode 100 and the first hole transport layer 321 so as to enhance the ability to inject holes into the first hole transport layer 321. The hole injection layer 310 may be selected from benzidine derivatives, starburst arylamine compounds, phthalocyanine derivatives, and other materials, and the present application is not particularly restricted in this respect. The material of the hole injection layer 310 is, for example, selected from the following compounds or any combinations thereof:

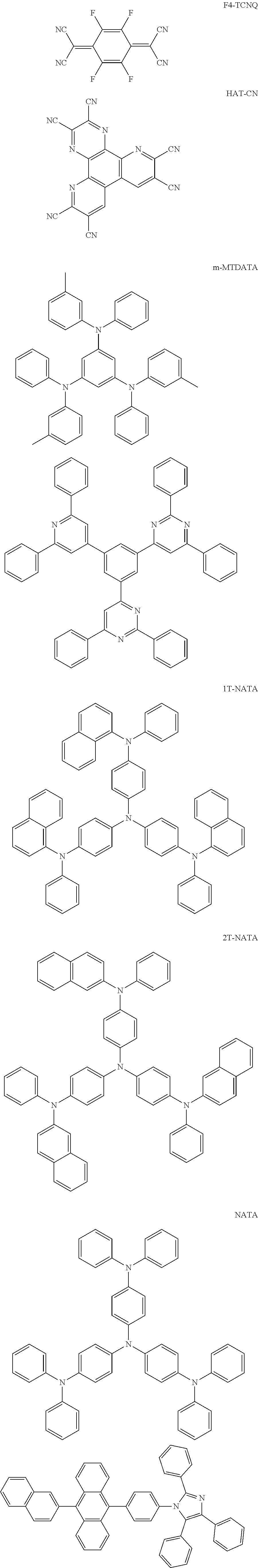

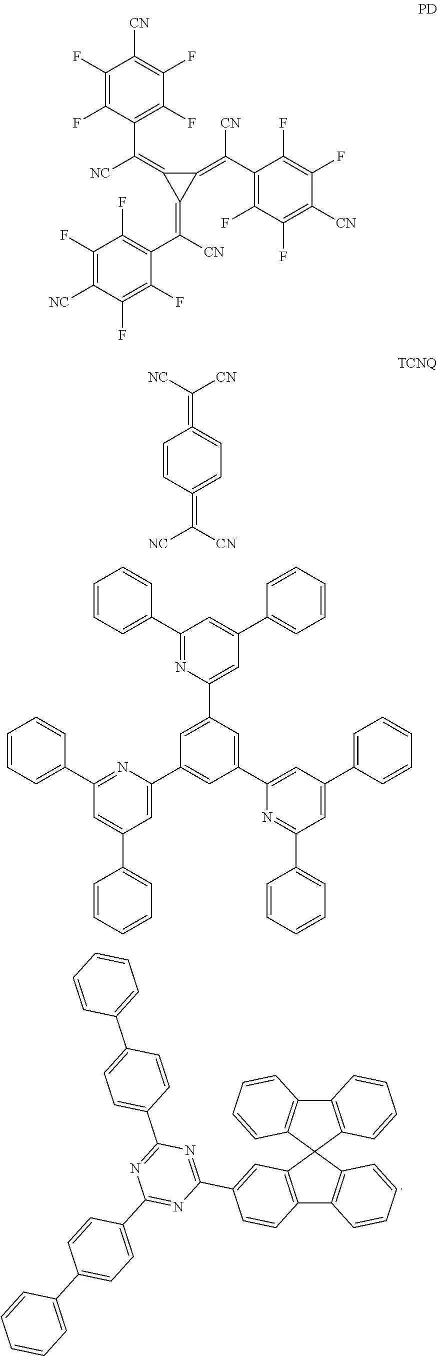

In an embodiment, the hole injection layer 310 is composed of PD.

In the present application, the organic light emitting layer 330 may be composed of a single light emitting material, or may comprise a host material and a guest material. Optionally, the organic light emitting layer 330 is composed of a host material and a guest material. Holes injected into the organic light emitting layer 330 and electrons injected into the organic light emitting layer 330 can recombine in the organic light emitting layer 330 to form excitons. The excitons transmit energy to the host material, and the host material transmits the energy to the guest material, thereby enabling the guest material to emit light.

The host material of the organic light emitting layer 330 may comprise metal chelating compounds, stilbene derivatives, aromatic amine derivatives, dibenzofuran derivatives, or other types of materials. Optionally, the host material comprises the nitrogen-containing compound of the present application.

The guest material of the organic light emitting layer 330 may be a compound having a condensed aryl ring or a derivative thereof, a compound having a heteroaryl ring or a derivative thereof, an aromatic amine derivative, or other materials, and the present application is not particularly restricted in this respect. The guest material is also known as a doping material or a dopant, which can be categorized, according to its type of luminescence, as a fluorescent dopant or a phosphorescent dopant. Specific examples of the phosphorescent dopant include, but are not limited to:

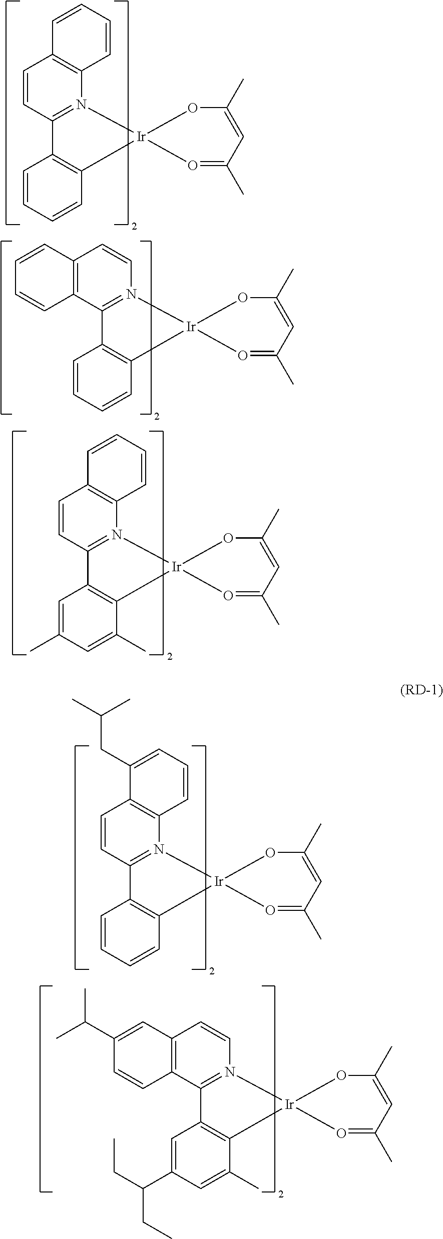

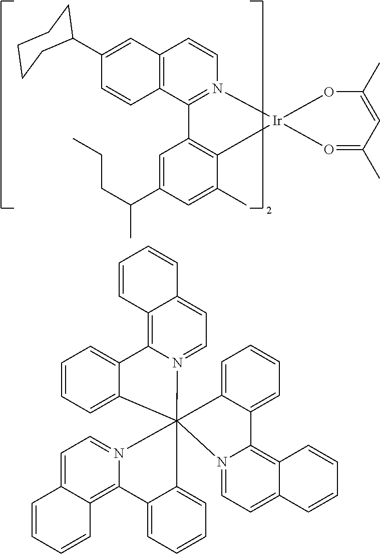

In an embodiment of the present application, the organic electroluminescent device is a red light organic electroluminescent device. In a more specific embodiment, the host material of the organic light emitting layer 330 comprises the nitrogen-containing compound of the present application. The guest material is, for example, RD-1.

In an embodiment of the present application, the organic electroluminescent device is a green light organic electroluminescent device. In a more specific embodiment, the host material of the organic light emitting layer 330 comprises the nitrogen-containing compound of the present application. The guest material is, for example, fac-Ir(ppy)₃.

The electron transport layer 340 may be a single-layer structure or a multi-layer structure, and may comprise one or more electron transport materials. The electron transport materials may be selected from, but are not limited to, BTB, LiQ, benzimidazole derivatives, oxadiazole derivatives, quinoxaline derivatives, or other electron transport materials, which are not particularly limited in the present application. The material of the electron transport layer 340 includes, but is not limited to, the following compounds:

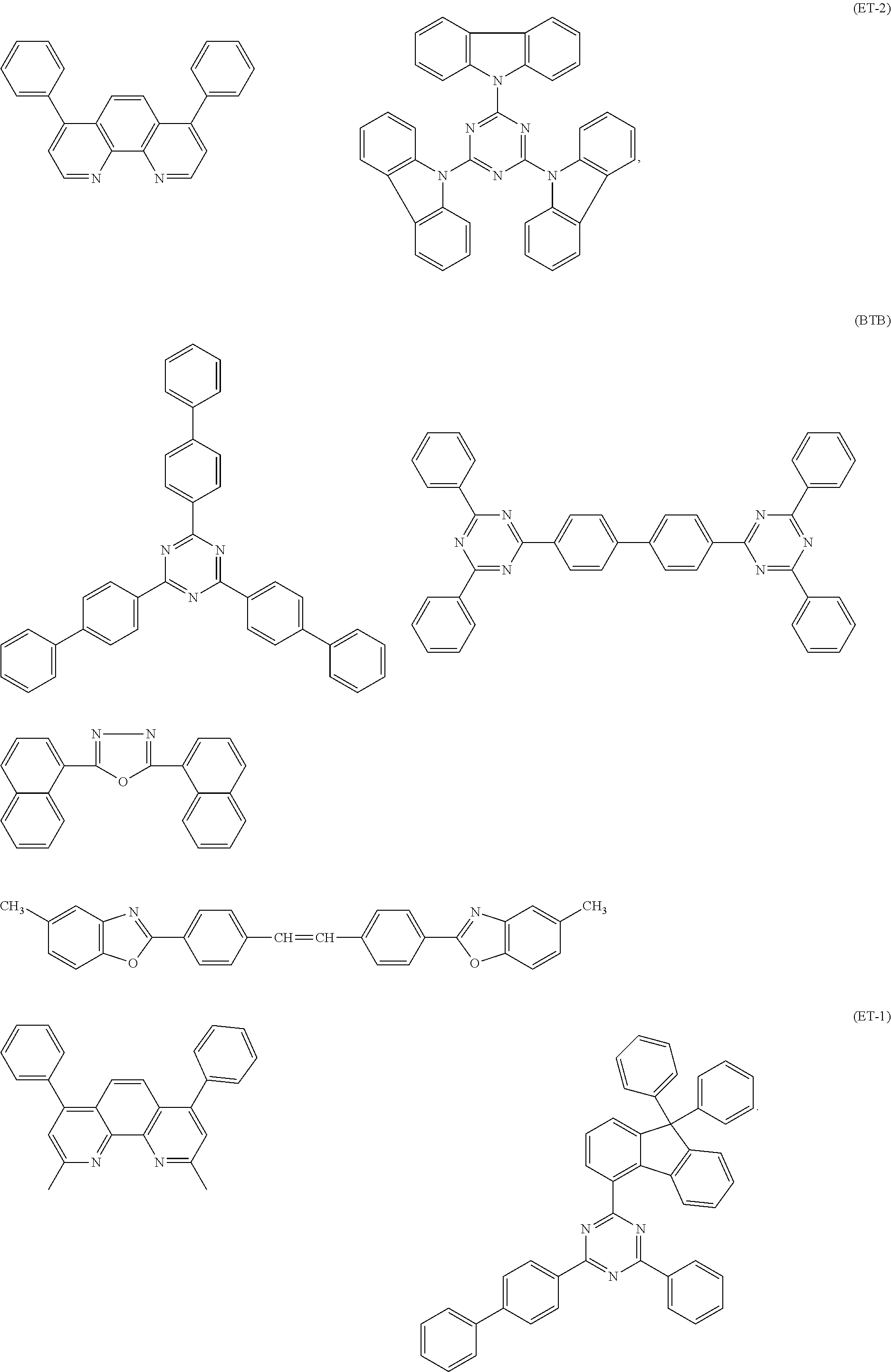

In an embodiment of the present application, the electron transport layer 340 is composed of ET-1 and LiQ, or composed of ET-2 and LiQ.

In the present application, the cathode 200 may comprise a cathode material, which is a low-work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, lead, and alloys thereof; or multilayer materials such as LiF/Al, Liq/Al, LiO₂/Al, LiF/Ca, LiF/Al, and BaF₂/Ca. Optionally, a metal electrode comprising magnesium and silver is included as the cathode.

Optionally, an electron injection layer 350 is further provided between the cathode 200 and the electron transport layer 340 so as to enhance the ability to inject electrons into the electron transport layer 340. The electron injection layer 350 may comprise an inorganic material such as an alkali metal sulfide, an alkali metal halide, and the like, or may comprise a complex of an alkali metal and an organic compound.

In an embodiment of the present application, the electron injection layer 350 may comprise ytterbium (Yb).

In a third aspect, the present application provides an electronic apparatus comprising the organic electroluminescent device described in the second aspect of the present application.

According to an embodiment, as shown in FIG. 2, the electronic apparatus provided is an electronic apparatus 400 including the organic electroluminescent device described above. The electronic apparatus 400 may be, for example, a display device, a lighting device, an optical communication device, or other type of electronic apparatus, including, but not limited to, for example, a computer screen, a mobile phone screen, a television, an electronic paper, an emergency lamp, an optical module, etc.

A synthesis method of the nitrogen-containing compound of the present application is described in detail below in conjunction with Synthesis Examples, but the present application is not limited thereto in any way.

### SYNTHESIS EXAMPLES

Those skilled in the art should appreciate that chemical reactions described in the present application may be used properly to prepare many organic compounds of the present application, and other methods that can be used to prepare the compounds of the present application are all considered to be within the scope of the present application. For example, the synthesis of those non-exemplary compounds of the present application may be successfully accomplished by those skilled in the art by modifying the method, for example, by properly protecting an interfering group, by utilizing other known reagents other than those described in the present application, or by making some conventional modifications to reaction conditions. Compounds for which a synthesis method is not mentioned in the present application are raw material products obtained commercially.

### Synthesis of 7-bromo-1-iodo-2-naphthalenethiol

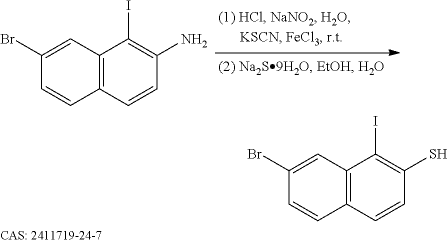 [SMILES: Nc1ccc2ccc(Br)cc2c1I ; Sc1ccc2ccc(Br)cc2c1I]

Under a nitrogen atmosphere, 7-bromo-1-iodo-2-naphthylamine (CAS: 2411719-24-7, 17.40 g, 50 mmol), concentrated hydrochloric acid (25 mL), and deionized water (25 mL) were added sequentially to a 1000 mL of three-neck flask. The resulting mixture was cooled to 0° C. in an ice water bath, followed by dropwise adding of an aqueous solution (25 mL) of sodium nitrite (3.45 g, 50 mmol), after the dropwise addition, then dropwise adding of an aqueous solution (25 mL) of potassium thiocyanate (9.72 g, 100 mmol) and ferric chloride (4.1 g, 25 mmol). After the dropwise addition, the resulting mixture was left to slowly warm up to room temperature for a reaction under stirring overnight. The reaction solution was poured into deionized water (200 mL), and extracted with dichloromethane (100 mL×3 times). The resulting organic phases were combined and dried with anhydrous sodium sulfate, followed by filtration and then distillation under reduced pressure to remove the solvent, obtaining a crude product. The crude product would be used directly in a next step without purification.

Under a nitrogen atmosphere, the resulting crude product, sodium sulfide nonahydrate (9.61 g, 100 mmol), ethanol (180 mL), and deionized water (360 mL) were added at one time to a 1000 mL of three-neck flask, and heated to reflux for a reaction under stirring for 16 hours. After being cooled to room temperature, the reaction system was filtered. The resulting filtrate was acidified with 1 M dilute hydrochloric acid to pH=2, and then extracted with dichloromethane (100 mL×3 times). The resulting organic phases were combined and dried with anhydrous sodium sulfate, followed by filtration and then distillation under reduced pressure to remove the solvent, obtaining a crude product. The crude product was purified by silica gel column chromatography with n-heptane as a mobile phase to yield 7-bromo-1-iodo-2-naphthalenethiol as a white solid (8.03 g, yield 44%).

### Synthesis of Sub-a1

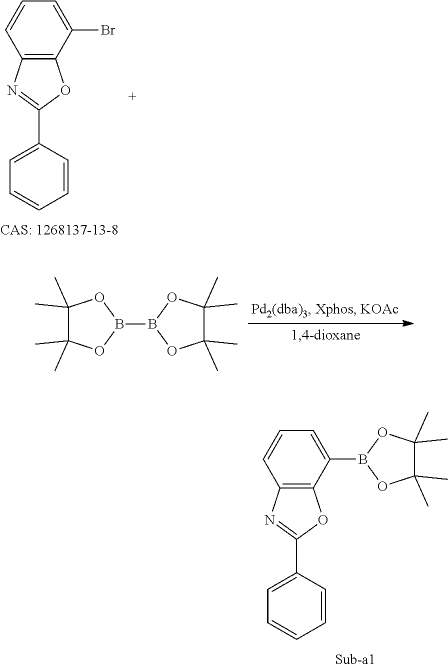

Under a nitrogen atmosphere, 7-bromo-2-phenylbenzoxazole (CAS: 1268137-13-8, 12.06 g, 44 mmol), bis(pinacolato)diboron (12.28 g, 48.4 mmol), potassium acetate (9.50 g, 96.8 mmol), and 1,4-dioxane (120 mL) were added sequentially to a 500 mL of three-neck flask, and stirred and heated. Once the resulting mixture was heated to 40° C., tris(dibenzylideneacetonyl)bis-palladium (Pd₂(dba)₃, 0.40 g, 0.44 mmol) and 2-dicyclohexylphosphino-2′,4′,6′-triisopropylbiphenyl (XPhos, 0.42 g, 0.88 mmol) were quickly added, followed by heating to reflux for a reaction under stirring overnight. After the reaction solution was cooled to room temperature, 200 mL of water was added. The resulting mixture was stirred thoroughly for 30 minutes, and filtered under reduced pressure. The resulting filter cake was washed with deionized water to neutral, and then rinsed with 100 mL of absolute ethanol to obtain crude product as a gray solid. The crude product was beaten with n-heptane once, and then dissolved in 200 mL of toluene. After the resulting solution became clear, the solution was passed through a silica gel column to remove the catalyst, yielding Sub-a1 as a white solid (10.17 g, yield 72%).

Sub-a2 to Sub-a4 were synthesized with reference to the synthesis of Sub-a1, with 7-bromo-2-phenylbenzoxazole being replaced with Reactant A shown in Table 1.

TABLE 1
Synthesis of Sub-a2 to Sub-a4

| Sub-a No. | Reactant A | Structure of Sub-a | Yield (%) |
|---|---|---|---|
| Sub-a2 | 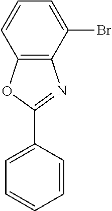 [SMILES: Brc1cccc2oc(-c3ccccc3)nc12] CAS: 1792993-81-7 | 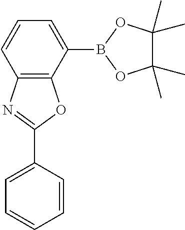 [SMILES: CC1(C)OB(c2cccc3nc(-c4ccccc4)oc23)OC1(C)C] | 64 |
| Sub-a3 | 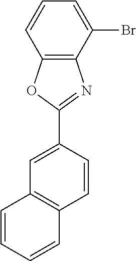 [SMILES: Brc1cccc2oc(-c3ccc4ccccc4c3)nc12] CAS: 2749511-90-6 | 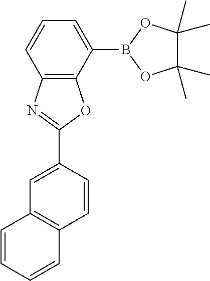 [SMILES: CC1(C)OB(c2cccc3nc(-c4ccc5ccccc5c4)oc23)OC1(C)C] | 66 |
| Sub-a4 | 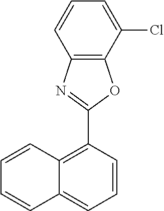 [SMILES: Clc1cccc2nc(-c3cccc4ccccc34)oc12] CAS: 2415412-21-2 | 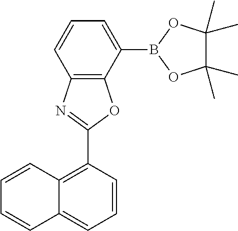 [SMILES: CC1(C)OB(c2cccc3nc(-c4cccc5ccccc45)oc23)OC1(C)C] | 62 |

### Synthesis of Sub-b1

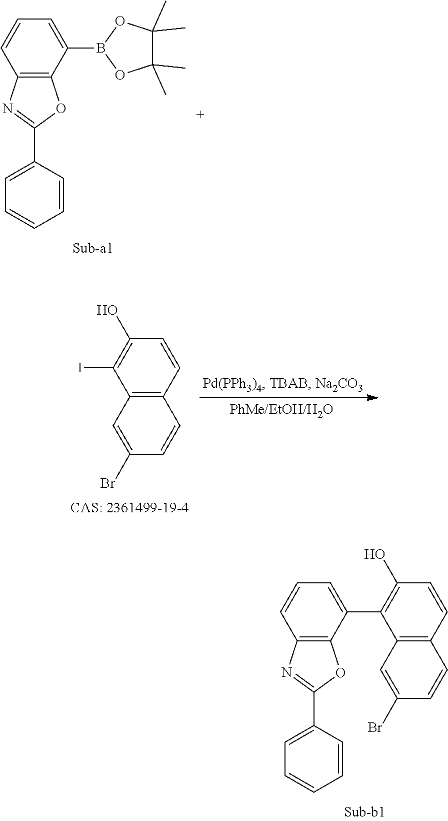 [SMILES: CC1(C)OB(c2cccc3nc(-c4ccccc4)oc23)OC1(C)C ; Oc1ccc2ccc(Br)cc2c1I ; Oc1ccc2ccc(Br)cc2c1-c1cccc2nc(-c3ccccc3)oc12]

Under a nitrogen atmosphere, Sub-a1 (17.66 g, 55 mmol), 7-bromo-1-iodo-2-hydroxynaphthalene (17.45 g, 50 mmol), tetrakis(triphenylphosphine)palladium (Pd(PPh₃)₄, 0.58 g, 0.5 mmol), anhydrous sodium carbonate (10.60 g, 100 mmol), toluene (180 mL), absolute ethanol (45 mL) and deionized water (45 mL) were added sequentially to a 500 mL of three-neck flask, stirred and heated to reflux for a reaction for 16 hours. After being cooled to room temperature, the resulting mixture was extracted with dichloromethane (150 mL×3 times). The resulting organic phases were combined and dried with anhydrous magnesium sulfate, followed by filtration and distillation under reduced pressure to remove the solvent, obtaining a crude product. The crude product was purified by silica gel column chromatography with n-heptane as a mobile phase to yield Sub-b1 as a white solid (11.03 g, yield 53%).

Sub-b2 to Sub-b13 were synthesized with reference to the synthesis of Sub-b1, with Sub-a1 being replaced with Reactant B shown in Table 2, and with 7-bromo-1-iodo-2-hydroxynaphthalene being replaced with Reactant C.

TABLE 2
Synthesis of Sub-b2 to Sub-b13

| Sub-b No. | Reactant B | Reactant C | Structure of Sub-b | Yield (%) |
|---|---|---|---|---|
| Sub-b2 | 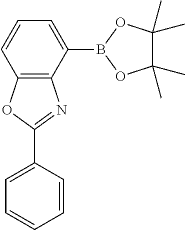 [SMILES: CC1(C)OB(c2cccc3oc(-c4ccccc4)nc23)OC1(C)C] | 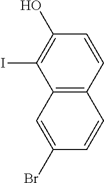 [SMILES: Oc1ccc2ccc(Br)cc2c1I] CAS: 2361499-19-4 | 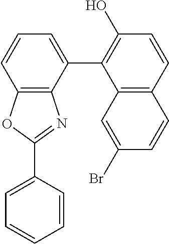 [SMILES: Oc1ccc2ccc(Br)cc2c1-c1cccc2oc(-c3ccccc3)nc12] | 49 |
| Sub-b3 | 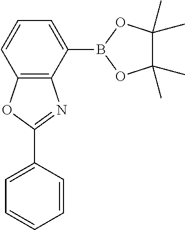 [SMILES: CC1(C)OB(c2cccc3oc(-c4ccccc4)nc23)OC1(C)C] | 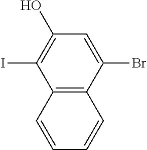 [SMILES: Oc1cc(Br)c2ccccc2c1I] CAS: 2364362-34-3 | 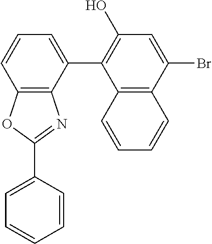 [SMILES: Oc1cc(Br)c2ccccc2c1-c1cccc2oc(-c3ccccc3)nc12] | 46 |
| Sub-b4 | 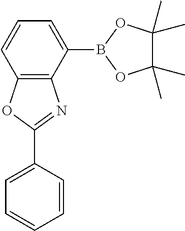 [SMILES: CC1(C)OB(c2cccc3oc(-c4ccccc4)nc23)OC1(C)C] | 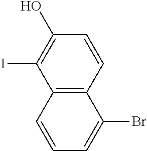 [SMILES: Oc1ccc2c(Br)cccc2c1I] CAS: 2411900-83-7 | 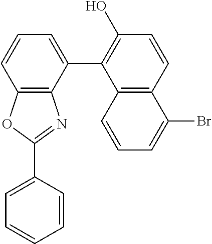 [SMILES: Oc1ccc2c(Br)cccc2c1-c1cccc2oc(-c3ccccc3)nc12] | 48 |
| Sub-b5 | 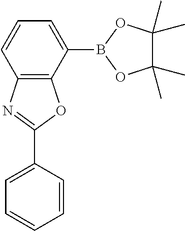 [SMILES: CC1(C)OB(c2cccc3nc(-c4ccccc4)oc23)OC1(C)C] | 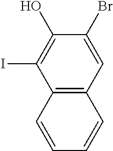 [SMILES: Oc1c(Br)cc2ccccc2c1I] CAS: 102154-09-6 | 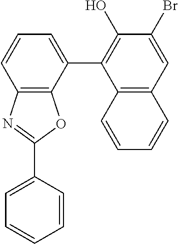 [SMILES: Oc1c(Br)cc2ccccc2c1-c1cccc2nc(-c3ccccc3)oc12] | 55 |
| Sub-b6 | 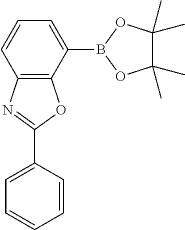 [SMILES: CC1(C)OB(c2cccc3nc(-c4ccccc4)oc23)OC1(C)C] | 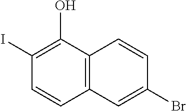 [SMILES: Oc1c(I)ccc2cc(Br)ccc12] CAS: 2364360-82-5 | 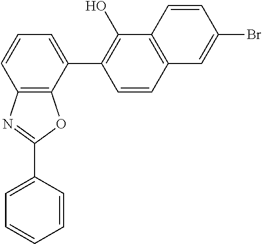 [SMILES: Oc1c(-c2cccc3nc(-c4ccccc4)oc23)ccc2cc(Br)ccc12] | 52 |
| Sub-b7 | 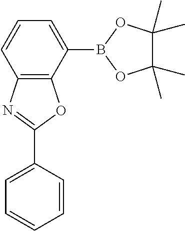 [SMILES: CC1(C)OB(c2cccc3nc(-c4ccccc4)oc23)OC1(C)C] | 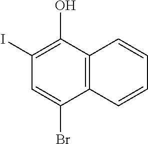 [SMILES: Oc1c(I)cc(Br)c2ccccc12] CAS: 1898230-03-9 | 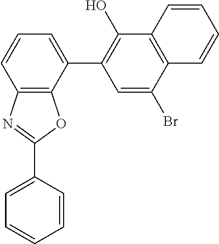 [SMILES: Oc1c(-c2cccc3nc(-c4ccccc4)oc23)cc(Br)c2ccccc12] | 51 |
| Sub-b8 | 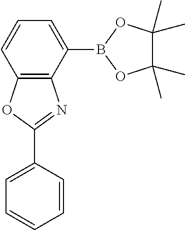 [SMILES: CC1(C)OB(c2cccc3oc(-c4ccccc4)nc23)OC1(C)C] | 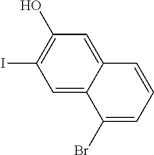 [SMILES: Oc1cc2cccc(Br)c2cc1I] CAS: 2364362-33-2 | 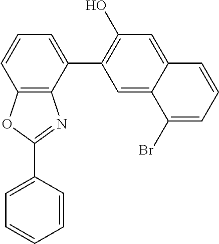 [SMILES: Oc1cc2cccc(Br)c2cc1-c1cccc2oc(-c3ccccc3)nc12] | 49 |
| Sub-b9 | 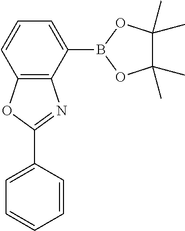 [SMILES: CC1(C)OB(c2cccc3oc(-c4ccccc4)nc23)OC1(C)C] | 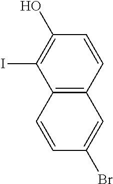 [SMILES: Oc1ccc2cc(Br)ccc2c1I] CAS: 102153-45-7 | 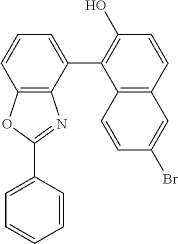 [SMILES: Oc1ccc2cc(Br)ccc2c1-c1cccc2oc(-c3ccccc3)nc12] | 51 |
| Sub-b10 | 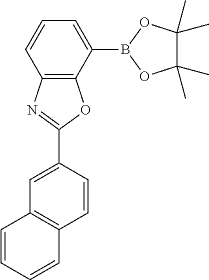 [SMILES: CC1(C)OB(c2cccc3nc(-c4ccc5ccccc5c4)oc23)OC1(C)C] | 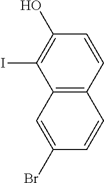 [SMILES: Oc1ccc2ccc(Br)cc2c1I] CAS: 2361499-19-4 | 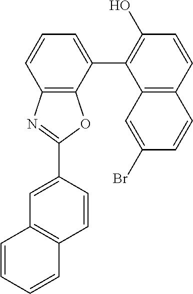 [SMILES: Oc1ccc2ccc(Br)cc2c1-c1cccc2nc(-c3ccc4ccccc4c3)oc12] | 45 |
| Sub-b11 | 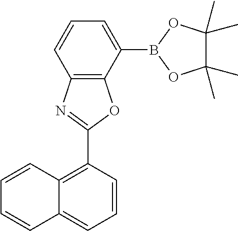 [SMILES: CC1(C)OB(c2cccc3nc(-c4cccc5ccccc45)oc23)OC1(C)C] | 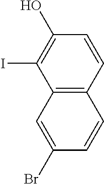 [SMILES: Oc1ccc2ccc(Br)cc2c1I] CAS: 2361499-19-4 | 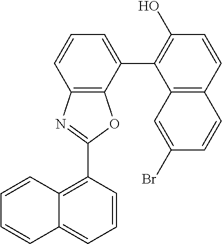 [SMILES: Oc1ccc2ccc(Br)cc2c1-c1cccc2nc(-c3cccc4ccccc34)oc12] | 42 |
| Sub-b12 | 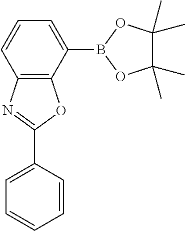 [SMILES: CC1(C)OB(c2cccc3nc(-c4ccccc4)oc23)OC1(C)C] | 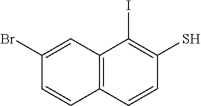 [SMILES: Sc1ccc2ccc(Br)cc2c1I] | 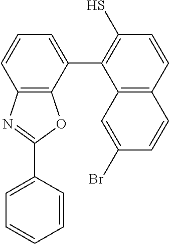 [SMILES: Sc1ccc2ccc(Br)cc2c1-c1cccc2nc(-c3ccccc3)oc12] | 42 |
| Sub-b13 | 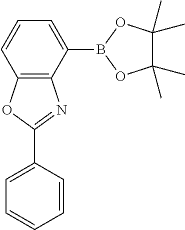 [SMILES: CC1(C)OB(c2cccc3oc(-c4ccccc4)nc23)OC1(C)C] | 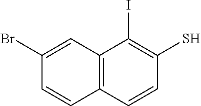 [SMILES: Sc1ccc2ccc(Br)cc2c1I] | 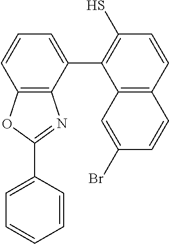 [SMILES: Sc1ccc2ccc(Br)cc2c1-c1cccc2oc(-c3ccccc3)nc12] | 43 |

### Synthesis of Sub-c1

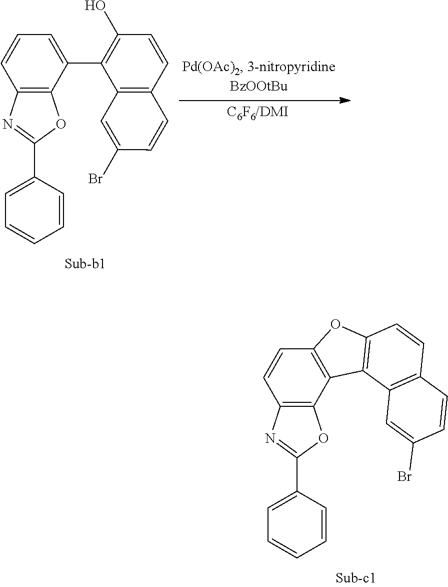 [SMILES: Oc1ccc2ccc(Br)cc2c1-c1cccc2nc(-c3ccccc3)oc12 ; Brc1ccc2ccc3oc4ccc5nc(-c6ccccc6)oc5c4c3c2c1]

Under a nitrogen atmosphere, Sub-b1 (20.81 g, 50 mmol), tert-butyl peroxybenzoate (BzOOt-Bu, 19.42 g, 100 mmol), palladium acetate (1.12 g, 5 mmol), 3-nitropyridine (0.62 g, 5 mmol), hexafluorobenzene (C₆F₆, 210 mL), and N,N′-dimethylimidazolidinone (DMI, 140 mL) were added sequentially to a 500 mL of three-neck flask, stirred and heated to 90° C. for a reaction for 4 hours. After being cooled to room temperature, the resulting mixture was extracted with ethyl acetate (100 mL×3 times). The resulting organic phase was dried with anhydrous magnesium sulfate, followed by filtration and distillation under reduced pressure to remove the solvent, obtaining a crude product. The crude product was purified by silica gel column chromatography with n-heptane/dichloromethane as a mobile phase to yield Sub-c1 as a white solid (10.77 g, yield 52%).

Sub-c2 to Sub-c11 were synthesized with reference to the synthesis of Sub-c1 with Sub-b1 being replaced with Reactant D shown in Table 3.

TABLE 3
Synthesis of Sub-c2 to Sub-c11

| Sub-c No. | Reactant D | Structure of Sub-c | Yield (%) |
|---|---|---|---|
| Sub-c2 | 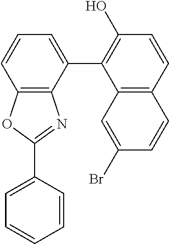 [SMILES: Oc1ccc2ccc(Br)cc2c1-c1cccc2oc(-c3ccccc3)nc12] | 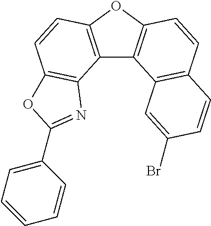 [SMILES: Brc1ccc2ccc3oc4ccc5oc(-c6ccccc6)nc5c4c3c2c1] | 66 |
| Sub-c3 | 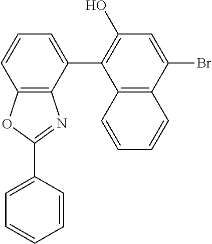 [SMILES: Oc1cc(Br)c2ccccc2c1-c1cccc2oc(-c3ccccc3)nc12] | 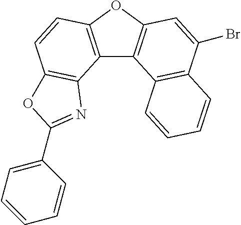 [SMILES: Brc1cc2oc3ccc4oc(-c5ccccc5)nc4c3c2c2ccccc12] | 58 |
| Sub-c4 | 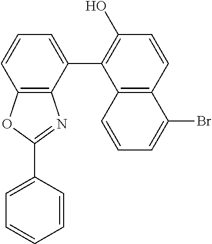 [SMILES: Oc1ccc2c(Br)cccc2c1-c1cccc2oc(-c3ccccc3)nc12] | 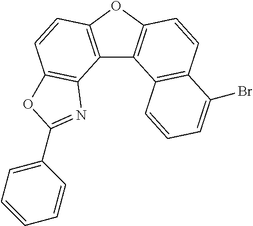 [SMILES: Brc1cccc2c1ccc1oc3ccc4oc(-c5ccccc5)nc4c3c12] | 60 |
| Sub-c5 | 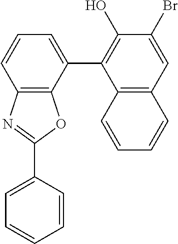 [SMILES: Oc1c(Br)cc2ccccc2c1-c1cccc2nc(-c3ccccc3)oc12] | 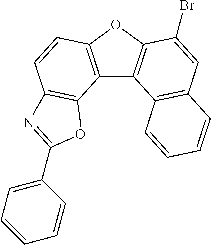 [SMILES: Brc1cc2ccccc2c2c1oc1ccc3nc(-c4ccccc4)oc3c12] | 61 |
| Sub-c6 | 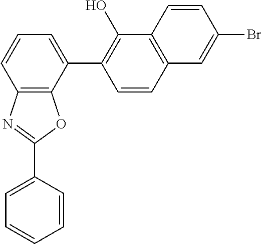 [SMILES: Oc1c(-c2cccc3nc(-c4ccccc4)oc23)ccc2cc(Br)ccc12] | 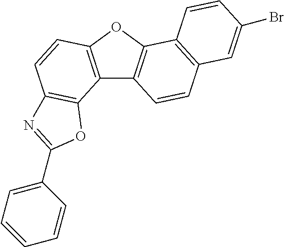 [SMILES: Brc1ccc2c(ccc3c2oc2ccc4nc(-c5ccccc5)oc4c23)c1] | 53 |
| Sub-c7 | 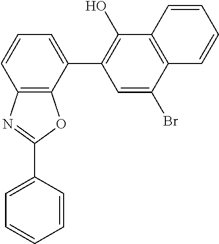 [SMILES: Oc1c(-c2cccc3nc(-c4ccccc4)oc23)cc(Br)c2ccccc12] | 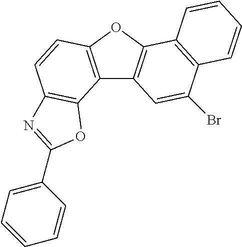 [SMILES: Brc1cc2c(oc3ccc4nc(-c5ccccc5)oc4c32)c2ccccc12] | 64 |
| Sub-c8 | 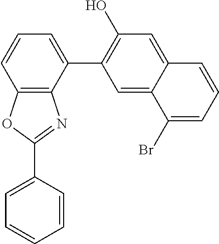 [SMILES: Oc1cc2cccc(Br)c2cc1-c1cccc2oc(-c3ccccc3)nc12] | 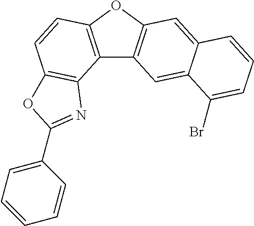 [SMILES: Brc1cccc2cc3oc4ccc5oc(-c6ccccc6)nc5c4c3cc12] | 54 |
| Sub-c9 | 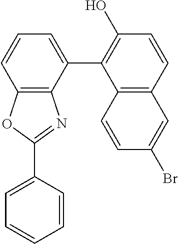 [SMILES: Oc1ccc2cc(Br)ccc2c1-c1cccc2oc(-c3ccccc3)nc12] | 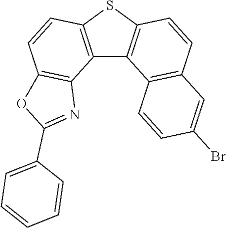 [SMILES: Brc1ccc2c(ccc3sc4ccc5oc(-c6ccccc6)nc5c4c32)c1] | 53 |
| Sub-c10 | 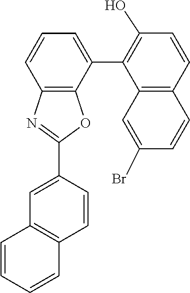 [SMILES: Oc1ccc2ccc(Br)cc2c1-c1cccc2nc(-c3ccc4ccccc4c3)oc12] | 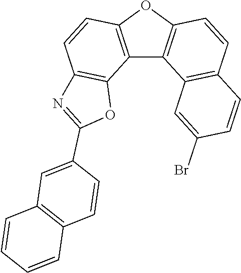 [SMILES: Brc1ccc2ccc3oc4ccc5nc(-c6ccc7ccccc7c6)oc5c4c3c2c1] | 56 |
| Sub-c11 | 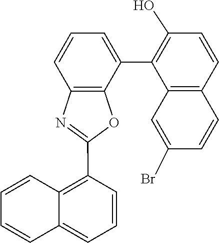 [SMILES: Oc1ccc2ccc(Br)cc2c1-c1cccc2nc(-c3cccc4ccccc34)oc12] | 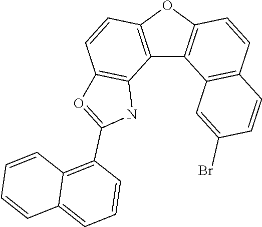 | 51 |

### Synthesis of Sub-c12

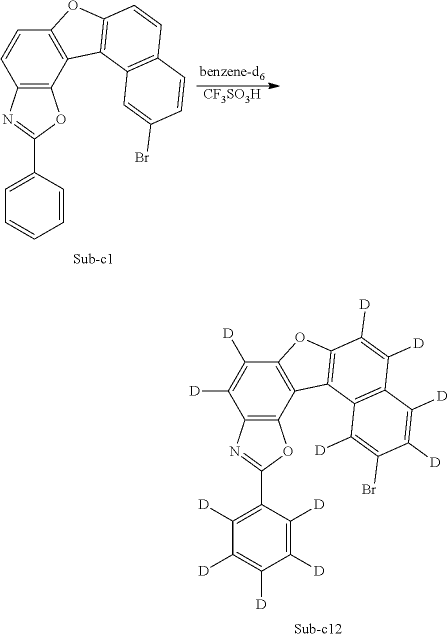

Under a nitrogen atmosphere, Sub-c1 (10.36 g, 25 mmol) and 200 mL of deuterated benzene-D6 were added to a 100 mL of three-neck flask, and heated to 60° C., followed by adding trifluoromethanesulfonic acid (22.51 g, 150 mmol), and then heating to boil for a reaction under stirring for 24 hours. After the reaction system was cooled to room temperature, 50 mL of heavy water was added, followed by stirring for 10 minutes, and then addition of a saturated aqueous solution of K₃PO₄ to neutralize the reaction solution. The resulting organic layers were extracted with dichloromethane (50 mL×3 times). The organic phases were combined and dried with anhydrous sodium sulfate, followed by filtration and then distillation under reduced pressure to remove the solvent, obtaining a crude product. The crude product was purified by silica gel column chromatography with n-heptane/dichloromethane as a mobile phase to yield Sub-c12 as a white solid (6.82 g, yield 64%).

### Synthesis of Sub-c13

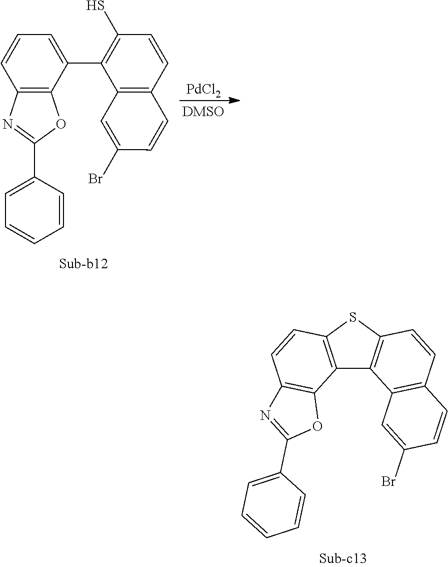

Under a nitrogen atmosphere, Sub-b12 (10.80 g, 25 mmol), palladium dichloride (0.22 g, 1.25 mmol), and DMSO (120 mL) were added to a 250 mL of three-neck flask, heated to 140° C. and stirred for 12 hours. After the reaction system was cooled to room temperature, the resulting organic layers were extracted with dichloromethane (50 mL×3 times). The organic phases were combined and dried with anhydrous sodium sulfate, followed by filtration and then distillation under reduced pressure to remove the solvent, obtaining a crude product. The crude product was purified by silica gel column chromatography with n-heptane/dichloromethane as a mobile phase to yield Sub-c13 as a white solid (7.85 g, yield 73%).

Sub-c14 was synthesized with reference to the synthesis of Sub-c13, with Sub-b12 being replaced with Reactant E shown in Table 4.

TABLE 4
Synthesis of Sub-c14

| Sub-c No. | Reactant E | Structure of Sub-c | Yield (%) |
|---|---|---|---|
| Sub-c14 | 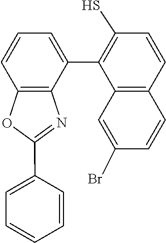 [SMILES: Sc1ccc2ccc(Br)cc2c1-c1cccc2oc(-c3ccccc3)nc12] | 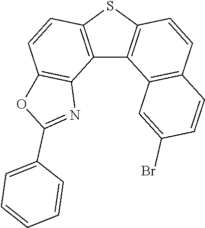 [SMILES: Brc1ccc2ccc3sc4ccc5oc(-c6ccccc6)nc5c4c3c2c1] | 69 |

### Synthesis of Sub-d1

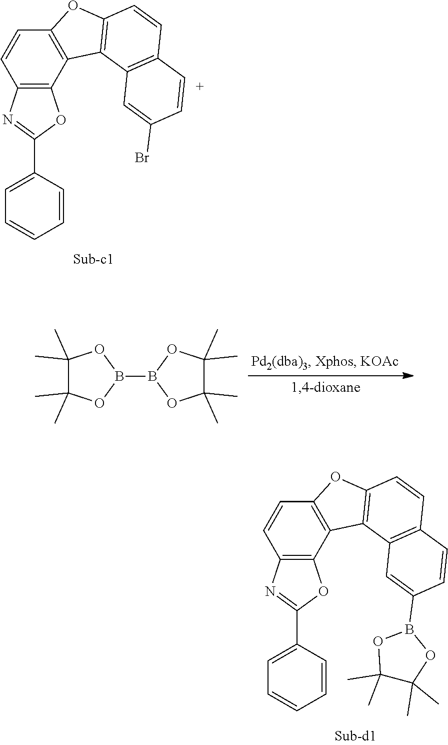

Under a nitrogen atmosphere, Sub-c1 (10.36 g, 25 mmol), bis(pinacolato)diboron (7.62 g, 30 mmol), potassium acetate (5.40 g, 55 mmol), and 1,4-dioxane (100 mL) were added sequentially to a 250 mL of three-neck flask, stirred and heated. Once the resulting mixture was heated to 40° C., tris(dibenzylideneacetonyl)bis-palladium (0.23 g, 0.25 mmol) and 2-dicyclohexylphosphino-2′,4′,6′-triisopropylbiphenyl (0.24 g, 0.5 mmol) were quickly added, followed by heating to reflux for a reaction under stirring overnight. After the reaction solution was cooled to room temperature, 100 mL of water was added. The resulting mixture was stirred thoroughly for 30 minutes, and filtered under reduced pressure. The resulting filter cake was washed with deionized water to neutral, and then rinsed with 50 mL of absolute ethanol to obtain crude product as a gray solid. The crude product was beaten with n-heptane once, and then dissolved in 100 mL of toluene. After the resulting solution became clear, the solution was passed through a silica gel column to remove the catalyst, yielding Sub-d1 as a white solid (7.15 g, yield 62%).

Sub-d2 to Sub-d14 were synthesized with reference to the synthesis of Sub-d1, with Sub-c1 being replaced with Reactant F shown in Table 5.

TABLE 5
Synthesis of Sub-d2 to Sub-d14

| Sub-d No. | Reactant F | Structure of Sub-d | Yield (%) |
|---|---|---|---|
| Sub-d2 | 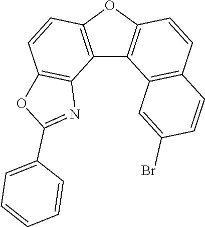 [SMILES: Brc1ccc2ccc3oc4ccc5oc(-c6ccccc6)nc5c4c3c2c1] | 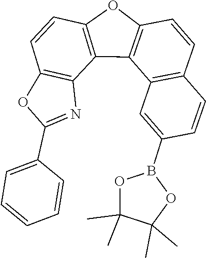 [SMILES: CC1(C)OB(c2ccc3ccc4oc5ccc6oc(-c7ccccc7)nc6c5c4c3c2)OC1(C)C] | 57 |
| Sub-d3 | 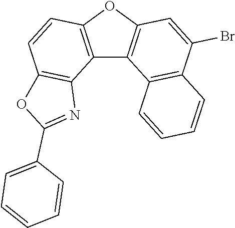 [SMILES: Brc1cc2oc3ccc4oc(-c5ccccc5)nc4c3c2c2ccccc12] | 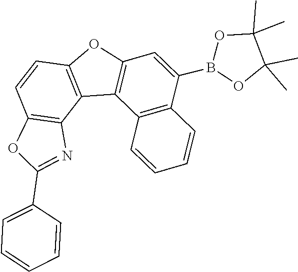 [SMILES: CC1(C)OB(c2cc3oc4ccc5oc(-c6ccccc6)nc5c4c3c3ccccc23)OC1(C)C] | 57 |
| Sub-d4 | 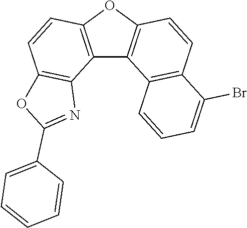 [SMILES: Brc1cccc2c1ccc1oc3ccc4oc(-c5ccccc5)nc4c3c12] | 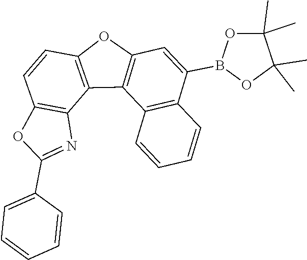 [SMILES: CC1(C)OB(c2cc3oc4ccc5oc(-c6ccccc6)nc5c4c3c3ccccc23)OC1(C)C] | 55 |
| Sub-d5 | 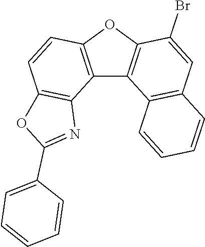 [SMILES: Brc1cc2ccccc2c2c1oc1ccc3oc(-c4ccccc4)nc3c12] | 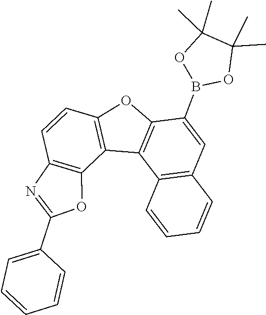 [SMILES: CC1(C)OB(c2cc3ccccc3c3c2oc2ccc4nc(-c5ccccc5)oc4c23)OC1(C)C] | 61 |
| Sub-d6 | 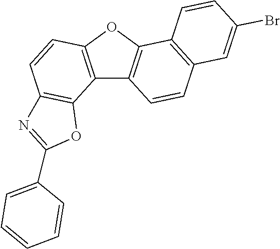 [SMILES: Brc1ccc2c(ccc3c2oc2ccc4nc(-c5ccccc5)oc4c23)c1] | 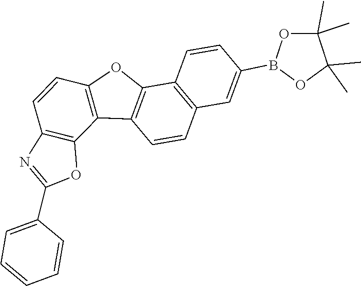 [SMILES: CC1(C)OB(c2ccc3c(ccc4c3oc3ccc5nc(-c6ccccc6)oc5c34)c2)OC1(C)C] | 56 |
| Sub-d7 | 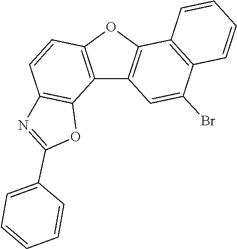 [SMILES: Brc1cc2c(oc3ccc4nc(-c5ccccc5)oc4c32)c2ccccc12] | 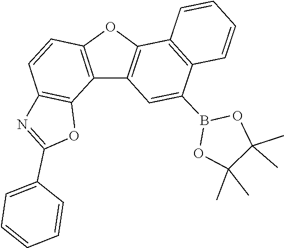 [SMILES: CC1(C)OB(c2cc3c(oc4ccc5nc(-c6ccccc6)oc5c43)c3ccccc23)OC1(C)C] | 50 |
| Sub-d8 | 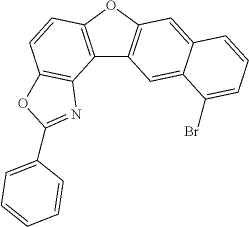 [SMILES: Brc1cccc2cc3oc4ccc5oc(-c6ccccc6)nc5c4c3cc12] | 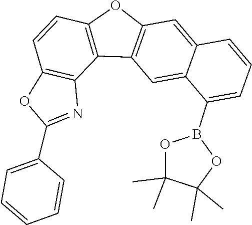 [SMILES: CC1(C)OB(c2cccc3cc4oc5ccc6oc(-c7ccccc7)nc6c5c4cc23)OC1(C)C] | 51 |
| Sub-d9 | 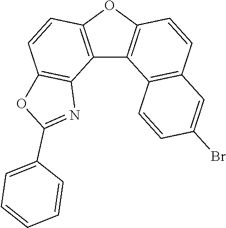 [SMILES: Brc1ccc2c(ccc3oc4ccc5oc(-c6ccccc6)nc5c4c32)c1] | 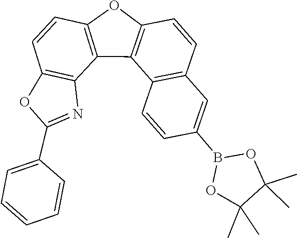 [SMILES: CC1(C)OB(c2ccc3c(ccc4oc5ccc6oc(-c7ccccc7)nc6c5c43)c2)OC1(C)C] | 51 |
| Sub-d10 | 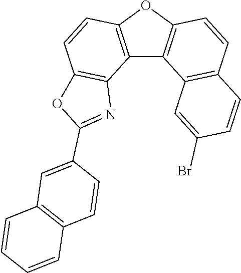 [SMILES: Brc1ccc2ccc3oc4ccc5oc(-c6ccc7ccccc7c6)nc5c4c3c2c1] | 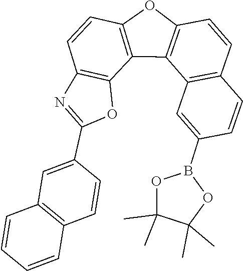 [SMILES: CC1(C)OB(c2ccc3ccc4oc5ccc6nc(-c7ccc8ccccc8c7)oc6c5c4c3c2)OC1(C)C] | 52 |
| Sub-d11 | 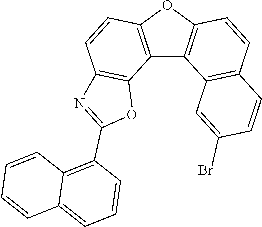 [SMILES: Brc1ccc2ccc3oc4ccc5nc(-c6cccc7ccccc67)oc5c4c3c2c1] | 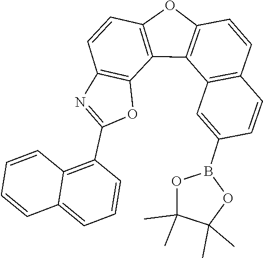 [SMILES: CC1(C)OB(c2ccc3ccc4oc5ccc6nc(-c7cccc8ccccc78)oc6c5c4c3c2)OC1(C)C] | 50 |
| Sub-d12 | 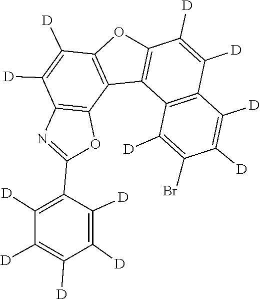 [SMILES: [2H]c1c([2H])c([2H])c(-c2nc3c([2H])c([2H])c4oc5c([2H])c([2H])c6c([2H])c([2H])c(Br)c([2H])c6c5c4c3o2)c([2H])c1[2H]] | 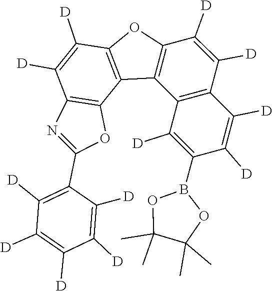 [SMILES: [2H]c1c([2H])c([2H])c(-c2nc3c([2H])c([2H])c4oc5c([2H])c([2H])c6c([2H])c([2H])c(B7OC(C)(C)C(C)(C)O7)c([2H])c6c5c4c3o2)c([2H])c1[2H]] | 45 |
| Sub-d13 | 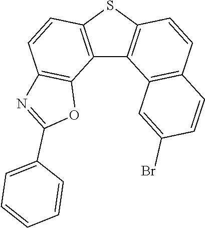 [SMILES: Brc1ccc2ccc3sc4ccc5nc(-c6ccccc6)oc5c4c3c2c1] | 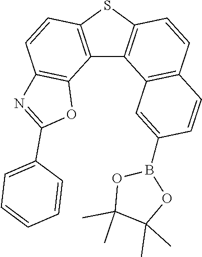 [SMILES: CC1(C)OB(c2ccc3ccc4sc5ccc6nc(-c7ccccc7)oc6c5c4c3c2)OC1(C)C] | 52 |
| Sub-d14 | 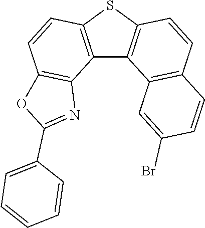 [SMILES: Brc1ccc2ccc3sc4ccc5oc(-c6ccccc6)nc5c4c3c2c1] | 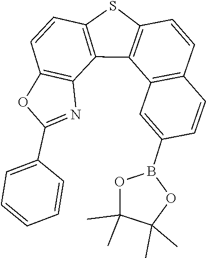 [SMILES: CC1(C)OB(c2ccc3ccc4sc5ccc6oc(-c7ccccc7)nc6c5c4c3c2)OC1(C)C] | 50 |

### Synthesis of Compound 1

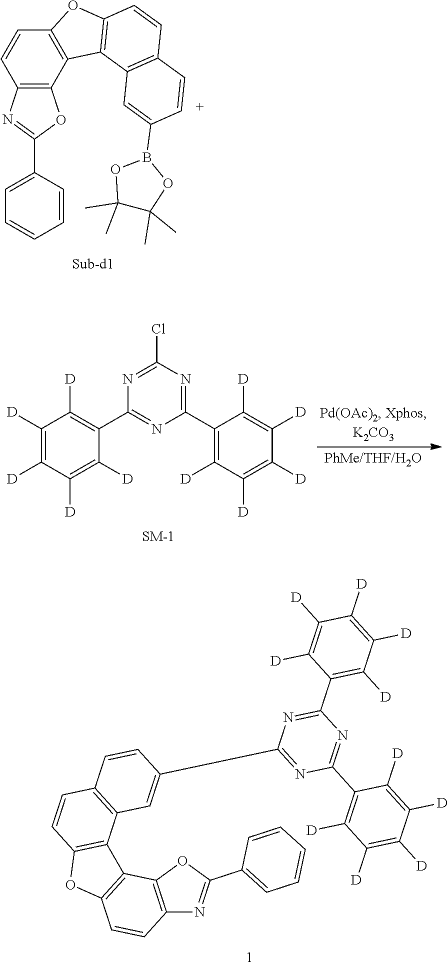 [SMILES: CC1(C)OB(c2ccc3ccc4oc5ccc6nc(-c7ccccc7)oc6c5c4c3c2)OC1(C)C ; [2H]c1c([2H])c([2H])c(-c2nc(Cl)nc(-c3c([2H])c([2H])c([2H])c([2H])c3[2H])n2)c([2H])c1[2H] ; [2H]c1c([2H])c([2H])c(-c2nc(-c3ccc4ccc5oc6ccc7nc(-c8ccccc8)oc7c6c5c4c3)nc(-c3c([2H])c([2H])c([2H])c([2H])c3[2H])n2)c([2H])c1[2H]]

Under a nitrogen atmosphere, Sub-d1 (11.53 g, 25 mmol), SM-1 (CAS: 1300115-09-6, 5.55 g, 20 mmol), palladium acetate (0.045 g, 0.2 mmol), 2-dicyclohexylphosphino-2′,4′,6′-triisopropylbiphenyl (0.19 g, 0.4 mmol), anhydrous potassium carbonate (5.53 g, 40 mmol), toluene (120 mL), tetrahydrofuran (30 mL), and deionized water (30 mL) were added sequentially to a 250 mL of three-neck flask, stirred and heated to reflux for a reaction for 16 hours. After being cooled to room temperature, the resulting mixture was extracted with dichloromethane (100 mL×3 times). The resulting organic phases were combined and dried with anhydrous magnesium sulfate, followed by filtration and then distillation under reduced pressure to remove the solvent, obtaining a crude product. The crude product was purified by silica gel column chromatography with dichloromethane/n-heptane as a mobile phase to yield Compound 1 as a white solid (8.99 g, yield 78%), m/z=577.2[M+H]⁺.

Compounds of the present application in Table 6 were synthesized with reference to the synthesis of Compound 1, with Sub-d1 being replaced with Reactant G shown in Table 6, SM-1 being replaced with Reactant H shown in Table 6.

TABLE 6
Synthesis of compounds of the present application

| | | | m/z | |
|---|---|---|---|---|
| | | Compound Structure and | ( [M + | Yield |
| Reactant G | Reactant H | Compound No. | H]⁺) | (%) |
| 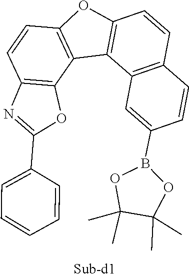 [SMILES: CC1(C)OB(c2ccc3ccc4oc5ccc6nc(-c7ccccc7)oc6c5c4c3c2)OC1(C)C] | 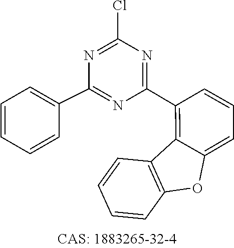 [SMILES: Clc1nc(-c2ccccc2)nc(-c2cccc3oc4ccccc4c23)n1] | 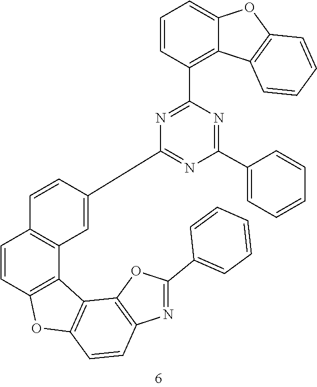 [SMILES: c1ccc(-c2nc(-c3ccc4ccc5oc6ccc7nc(-c8ccccc8)oc7c6c5c4c3)nc(-c3cccc4oc5ccccc5c34)n2)cc1] | 657.2 | 73 |
| 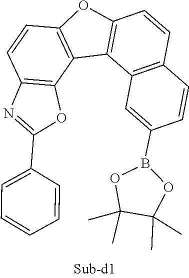 [SMILES: CC1(C)OB(c2ccc3ccc4oc5ccc6nc(-c7ccccc7)oc6c5c4c3c2)OC1(C)C] | 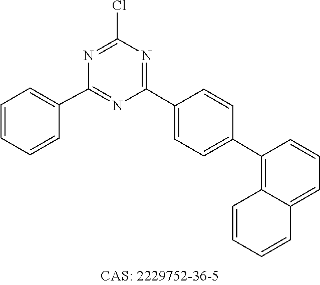 [SMILES: Clc1nc(-c2ccccc2)nc(-c2ccc(-c3cccc4ccccc34)cc2)n1] | 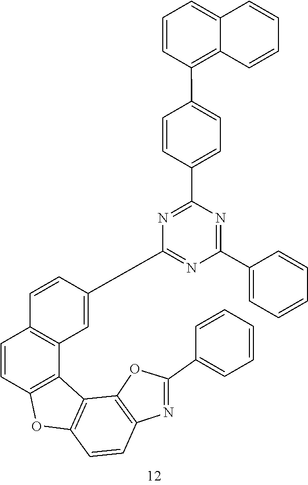 [SMILES: c1ccc(-c2nc(-c3ccc(-c4cccc5ccccc45)cc3)nc(-c3ccc4ccc5oc6ccc7nc(-c8ccccc8)oc7c6c5c4c3)n2)cc1] | 693.3 | 69 |
| 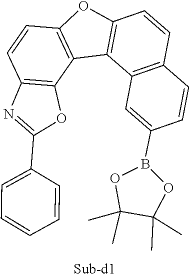 [SMILES: CC1(C)OB(c2ccc3ccc4oc5ccc6nc(-c7ccccc7)oc6c5c4c3c2)OC1(C)C] | 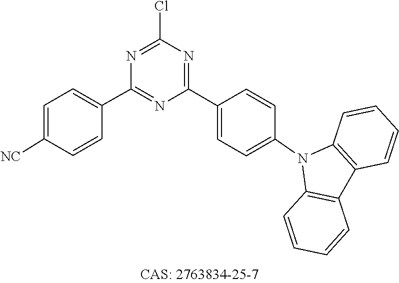 [SMILES: N#Cc1ccc(-c2nc(Cl)nc(-c3ccc(-n4c5ccccc5c5ccccc54)cc3)n2)cc1] | 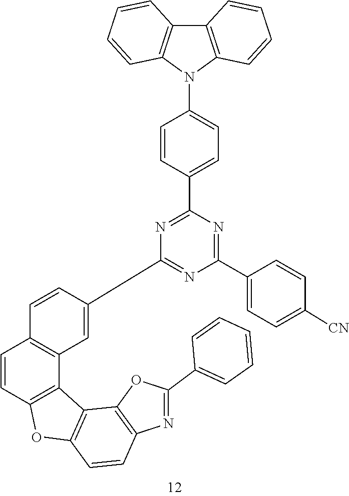 [SMILES: N#Cc1ccc(-c2nc(-c3ccc(-n4c5ccccc5c5ccccc54)cc3)nc(-c3ccc4ccc5oc6ccc7nc(-c8ccccc8)oc7c6c5c4c3)n2)cc1] | 757.2 | 67 |
| 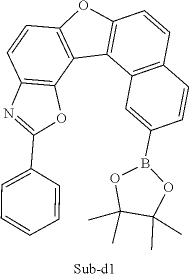 [SMILES: CC1(C)OB(c2ccc3ccc4oc5ccc6nc(-c7ccccc7)oc6c5c4c3c2)OC1(C)C] | 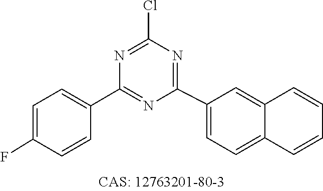 [SMILES: Fc1ccc(-c2nc(Cl)nc(-c3ccc4ccccc4c3)n2)cc1] | 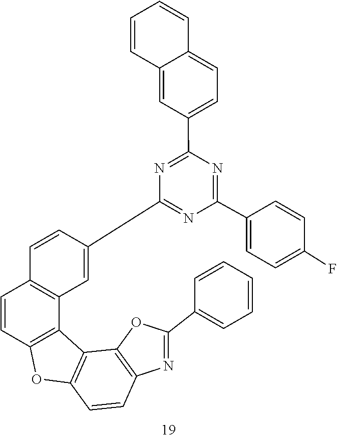 [SMILES: Fc1ccc(-c2nc(-c3ccc4ccccc4c3)nc(-c3ccc4ccc5oc6ccc7nc(-c8ccccc8)oc7c6c5c4c3)n2)cc1] | 635.2 | 68 |
| 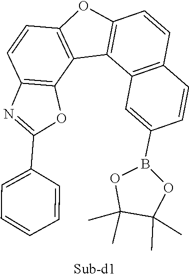 [SMILES: CC1(C)OB(c2ccc3ccc4oc5ccc6nc(-c7ccccc7)oc6c5c4c3c2)OC1(C)C] | 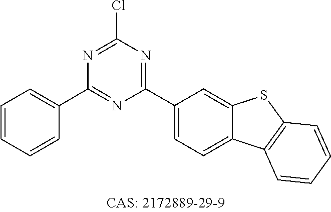 [SMILES: Clc1nc(-c2ccccc2)nc(-c2ccc3c(c2)sc2ccccc23)n1] | 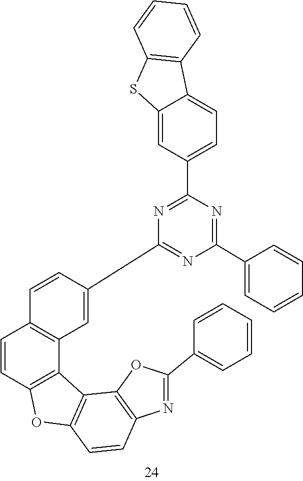 [SMILES: c1ccc(-c2nc(-c3ccc4c(c3)sc3ccccc34)nc(-c3ccc4ccc5oc6ccc7nc(-c8ccccc8)oc7c6c5c4c3)n2)cc1] | 673.2 | 72 |
| 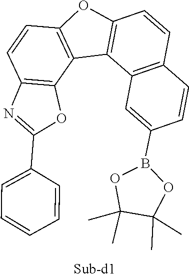 [SMILES: CC1(C)OB(c2ccc3ccc4oc5ccc6nc(-c7ccccc7)oc6c5c4c3c2)OC1(C)C] | 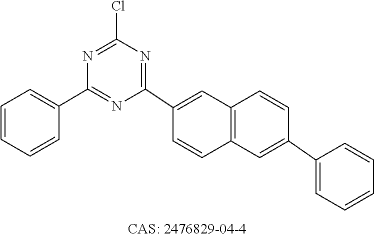 [SMILES: Clc1nc(-c2ccccc2)nc(-c2ccc3cc(-c4ccccc4)ccc3c2)n1] | 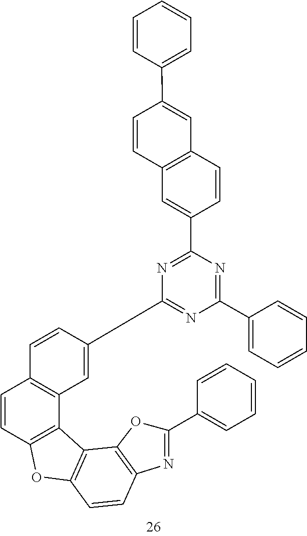 [SMILES: c1ccc(-c2ccc3cc(-c4nc(-c5ccccc5)nc(-c5ccc6ccc7oc8ccc9nc(-c%10ccccc%10)oc9c8c7c6c5)n4)ccc3c2)cc1] | 693.2 | 70 |
| 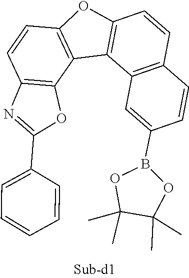 [SMILES: CC1(C)OB(c2ccc3ccc4oc5ccc6nc(-c7ccccc7)oc6c5c4c3c2)OC1(C)C] | 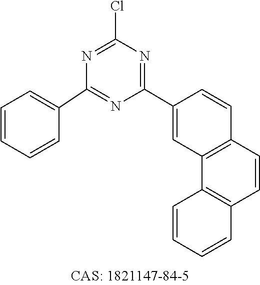 [SMILES: Clc1nc(-c2ccccc2)nc(-c2ccc3ccc4ccccc4c3c2)n1] | 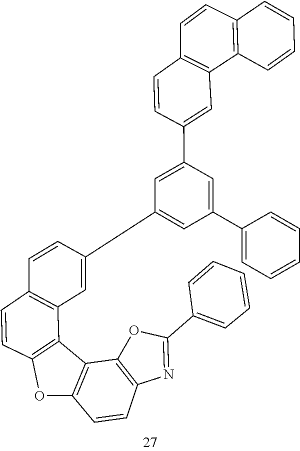 [SMILES: c1ccc(-c2cc(-c3ccc4ccc5ccccc5c4c3)cc(-c3ccc4ccc5oc6ccc7nc(-c8ccccc8)oc7c6c5c4c3)c2)cc1] | 667.2 | 71 |
| 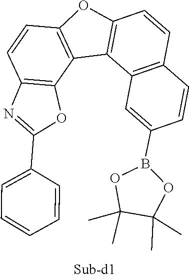 [SMILES: CC1(C)OB(c2ccc3ccc4oc5ccc6nc(-c7ccccc7)oc6c5c4c3c2)OC1(C)C] | 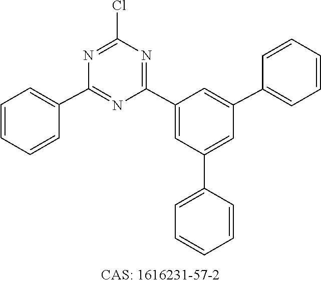 [SMILES: Clc1nc(-c2ccccc2)nc(-c2cc(-c3ccccc3)cc(-c3ccccc3)c2)n1] | 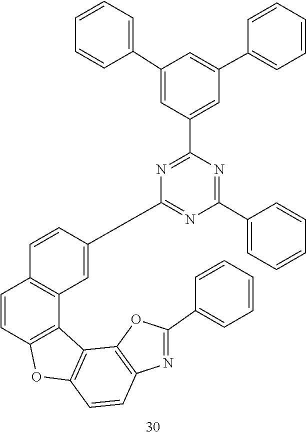 [SMILES: c1ccc(-c2cc(-c3ccccc3)cc(-c3nc(-c4ccccc4)nc(-c4ccc5ccc6oc7ccc8nc(-c9ccccc9)oc8c7c6c5c4)n3)c2)cc1] | 719.3 | 70 |
| 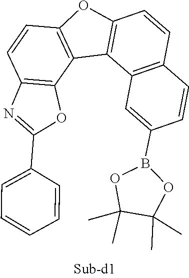 [SMILES: CC1(C)OB(c2ccc3ccc4oc5ccc6nc(-c7ccccc7)oc6c5c4c3c2)OC1(C)C] | 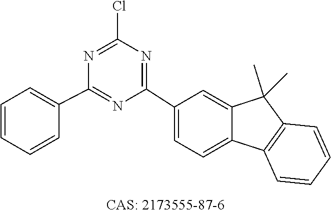 [SMILES: CC1(C)c2ccccc2-c2ccc(-c3nc(Cl)nc(-c4ccccc4)n3)cc21] | 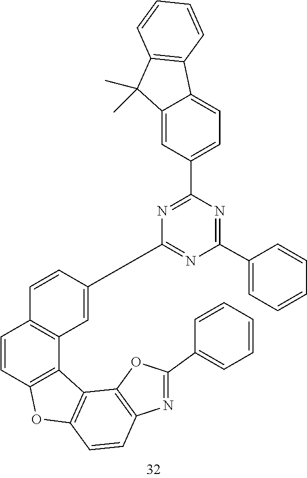 [SMILES: CC1(C)c2ccccc2-c2ccc(-c3nc(-c4ccccc4)nc(-c4ccc5ccc6oc7ccc8nc(-c9ccccc9)oc8c7c6c5c4)n3)cc21] | 683.2 | 73 |
| 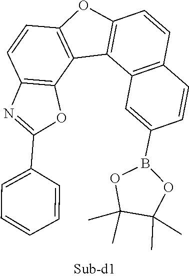 [SMILES: CC1(C)OB(c2ccc3ccc4oc5ccc6nc(-c7ccccc7)oc6c5c4c3c2)OC1(C)C] | 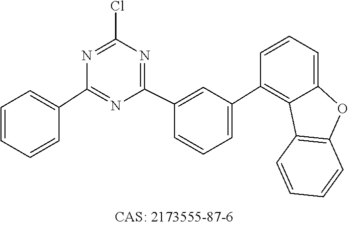 [SMILES: Clc1nc(-c2ccccc2)nc(-c2cccc(-c3cccc4oc5ccccc5c34)c2)n1] | 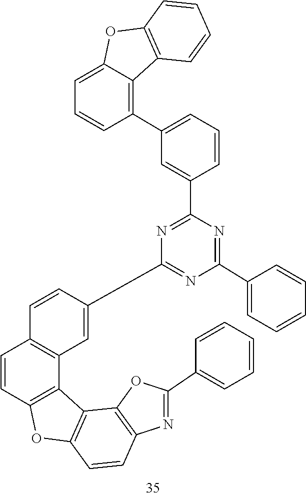 [SMILES: c1ccc(-c2nc(-c3cccc(-c4cccc5oc6ccccc6c45)c3)nc(-c3ccc4ccc5oc6ccc7nc(-c8ccccc8)oc7c6c5c4c3)n2)cc1] | 733.2 | 65 |
| 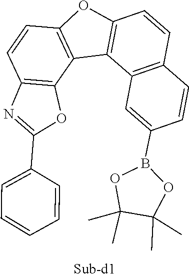 [SMILES: CC1(C)OB(c2ccc3ccc4oc5ccc6nc(-c7ccccc7)oc6c5c4c3c2)OC1(C)C] | 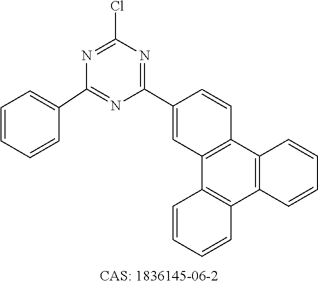 [SMILES: Clc1nc(-c2ccccc2)nc(-c2ccc3c4ccccc4c4ccccc4c3c2)n1] | 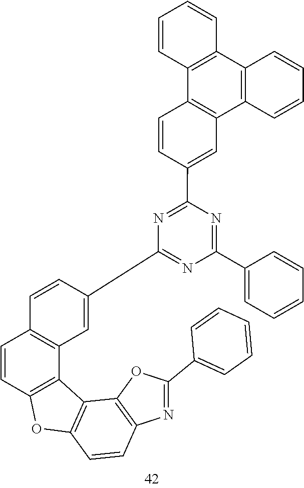 [SMILES: c1ccc(-c2nc(-c3ccc4c5ccccc5c5ccccc5c4c3)nc(-c3ccc4ccc5oc6ccc7nc(-c8ccccc8)oc7c6c5c4c3)n2)cc1] | 717.2 | 70 |
| 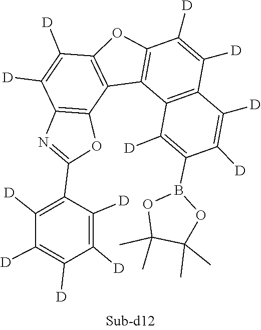 [SMILES: [2H]c1c([2H])c([2H])c(-c2nc3c([2H])c([2H])c4oc5c([2H])c([2H])c6c([2H])c([2H])c(B7OC(C)(C)C(C)(C)O7)c([2H])c6c5c4c3o2)c([2H])c1[2H]] | 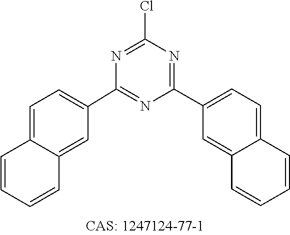 [SMILES: Clc1nc(-c2ccc3ccccc3c2)nc(-c2ccc3ccccc3c2)n1] | 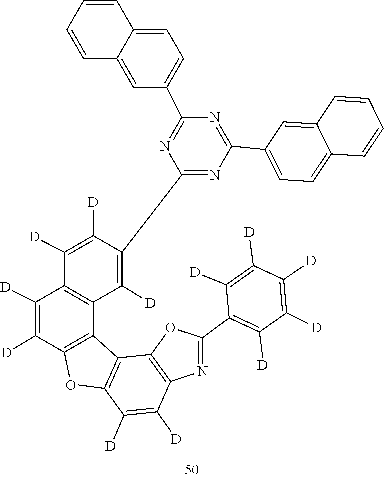 [SMILES: [2H]c1c([2H])c([2H])c(-c2nc3c([2H])c([2H])c4oc5c([2H])c([2H])c6c([2H])c([2H])c(-c7nc(-c8ccc9ccccc9c8)nc(-c8ccc9ccccc9c8)n7)c([2H])c6c5c4c3o2)c([2H])c1[2H]] | 679.3 | 69 |
| 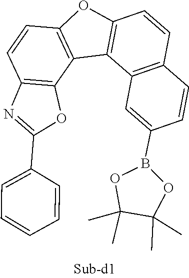 [SMILES: CC1(C)OB(c2ccc3ccc4oc5ccc6nc(-c7ccccc7)oc6c5c4c3c2)OC1(C)C] | 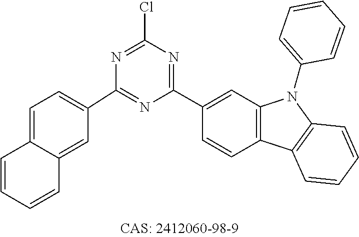 [SMILES: Clc1nc(-c2ccc3ccccc3c2)nc(-c2ccc3c4ccccc4n(-c4ccccc4)c3c2)n1] | 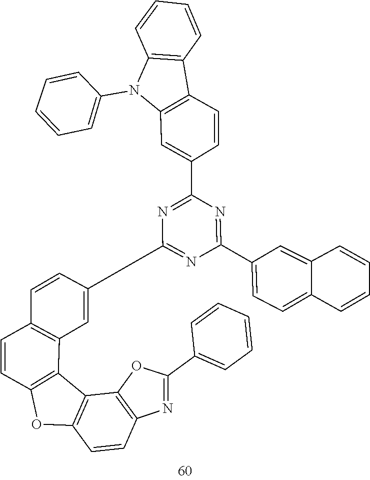 [SMILES: c1ccc(-c2nc3ccc4oc5ccc6ccc(-c7nc(-c8ccc9ccccc9c8)nc(-c8ccc9c%10ccccc%10n(-c%10ccccc%10)c9c8)n7)cc6c5c4c3o2)cc1] | 782.3 | 68 |
| 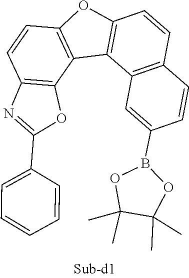 [SMILES: CC1(C)OB(c2ccc3ccc4oc5ccc6nc(-c7ccccc7)oc6c5c4c3c2)OC1(C)C] | 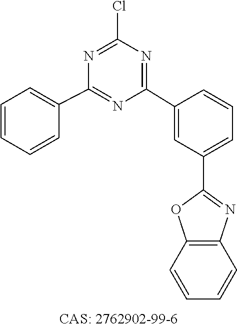 [SMILES: Clc1nc(-c2ccccc2)nc(-c2cccc(-c3nc4ccccc4o3)c2)n1] | 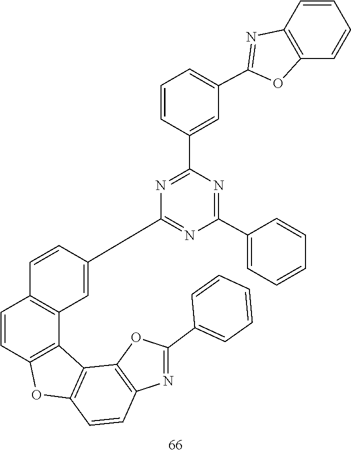 [SMILES: c1ccc(-c2nc(-c3cccc(-c4nc5ccccc5o4)c3)nc(-c3ccc4ccc5oc6ccc7nc(-c8ccccc8)oc7c6c5c4c3)n2)cc1] | 684.2 | 65 |
| 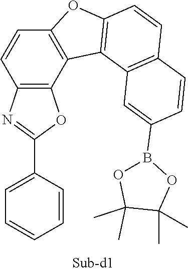 [SMILES: CC1(C)OB(c2ccc3ccc4oc5ccc6nc(-c7ccccc7)oc6c5c4c3c2)OC1(C)C] | 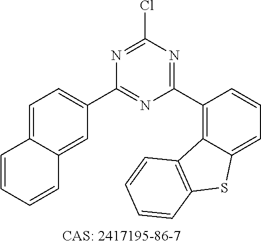 [SMILES: Clc1nc(-c2ccc3ccccc3c2)nc(-c2cccc3sc4ccccc4c23)n1] | 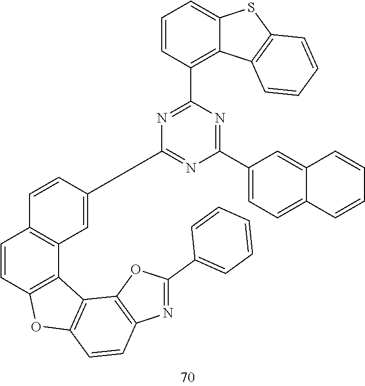 [SMILES: c1ccc(-c2nc3ccc4oc5ccc6ccc(-c7nc(-c8ccc9ccccc9c8)nc(-c8cccc9sc%10ccccc%10c89)n7)cc6c5c4c3o2)cc1] | 723.2 | 73 |
| 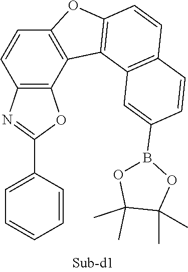 [SMILES: CC1(C)OB(c2ccc3ccc4oc5ccc6nc(-c7ccccc7)oc6c5c4c3c2)OC1(C)C] | 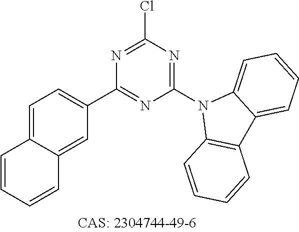 [SMILES: Clc1nc(-c2ccc3ccccc3c2)nc(-n2c3ccccc3c3ccccc32)n1] | 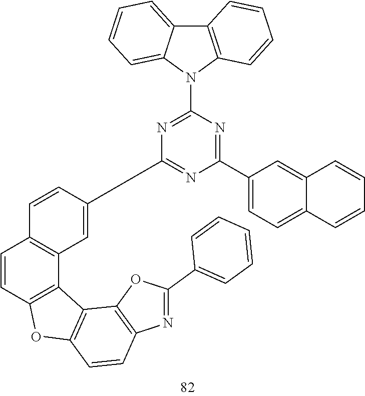 [SMILES: c1ccc(-c2nc3ccc4oc5ccc6ccc(-c7nc(-c8ccc9ccccc9c8)nc(-n8c9ccccc9c9ccccc98)n7)cc6c5c4c3o2)cc1] | 706.2 | 74 |
| 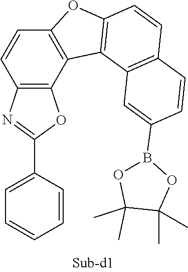 [SMILES: CC1(C)OB(c2ccc3ccc4oc5ccc6nc(-c7ccccc7)oc6c5c4c3c2)OC1(C)C] | 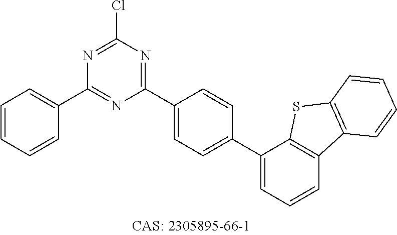 [SMILES: Clc1nc(-c2ccccc2)nc(-c2ccc(-c3cccc4c3sc3ccccc34)cc2)n1] | 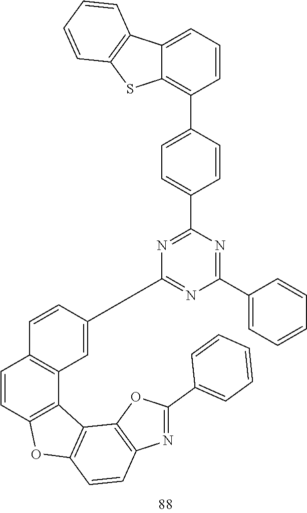 [SMILES: c1ccc(-c2nc(-c3ccc(-c4cccc5c4sc4ccccc45)cc3)nc(-c3ccc4ccc5oc6ccc7nc(-c8ccccc8)oc7c6c5c4c3)n2)cc1] | 749.2 | 61 |
| 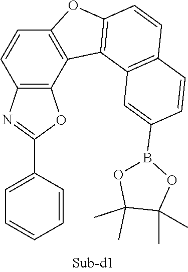 [SMILES: CC1(C)OB(c2ccc3ccc4oc5ccc6nc(-c7ccccc7)oc6c5c4c3c2)OC1(C)C] | 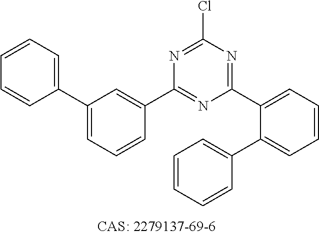 [SMILES: Clc1nc(-c2cccc(-c3ccccc3)c2)nc(-c2ccccc2-c2ccccc2)n1] | 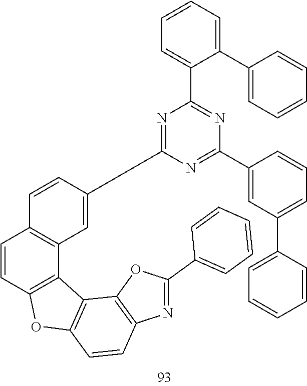 [SMILES: c1ccc(-c2cccc(-c3nc(-c4ccc5ccc6oc7ccc8nc(-c9ccccc9)oc8c7c6c5c4)nc(-c4ccccc4-c4ccccc4)n3)c2)cc1] | 719.2 | 62 |
| 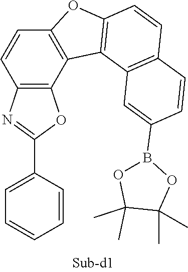 [SMILES: CC1(C)OB(c2ccc3ccc4oc5ccc6nc(-c7ccccc7)oc6c5c4c3c2)OC1(C)C] | 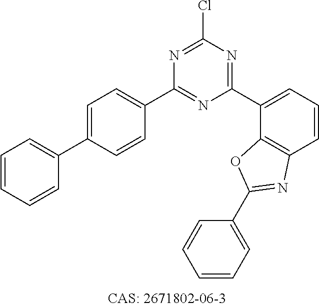 [SMILES: Clc1nc(-c2ccc(-c3ccccc3)cc2)nc(-c2cccc3nc(-c4ccccc4)oc23)n1] | 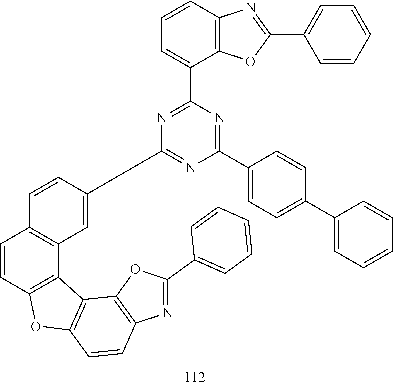 [SMILES: c1ccc(-c2ccc(-c3nc(-c4ccc5ccc6oc7ccc8nc(-c9ccccc9)oc8c7c6c5c4)nc(-c4cccc5nc(-c6ccccc6)oc45)n3)cc2)cc1] | 760.2 | 64 |
| 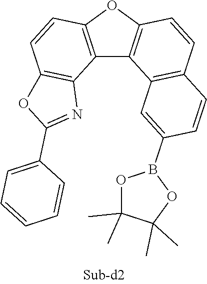 [SMILES: CC1(C)OB(c2ccc3ccc4oc5ccc6oc(-c7ccccc7)nc6c5c4c3c2)OC1(C)C] | 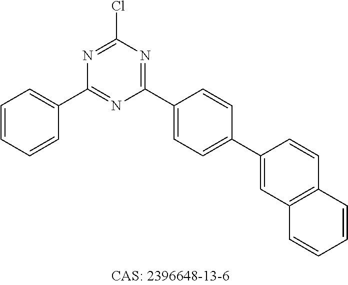 [SMILES: Clc1nc(-c2ccccc2)nc(-c2ccc(-c3ccc4ccccc4c3)cc2)n1] | 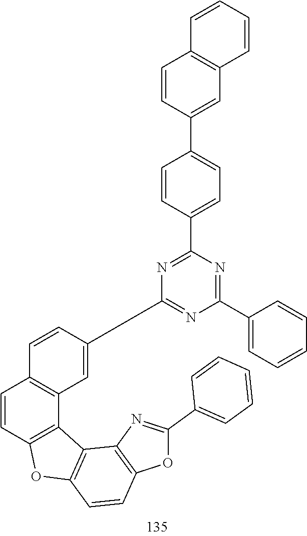 [SMILES: c1ccc(-c2nc(-c3ccc(-c4ccc5ccccc5c4)cc3)nc(-c3ccc4ccc5oc6ccc7oc(-c8ccccc8)nc7c6c5c4c3)n2)cc1] | 693.2 | 68 |
| 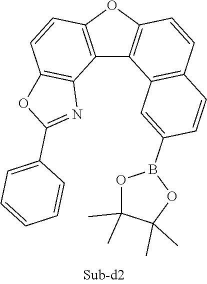 [SMILES: CC1(C)OB(c2ccc3ccc4oc5ccc6oc(-c7ccccc7)nc6c5c4c3c2)OC1(C)C] | 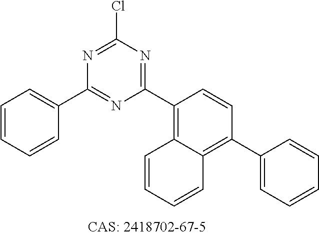 [SMILES: Clc1nc(-c2ccccc2)nc(-c2ccc(-c3ccccc3)c3ccccc23)n1] | 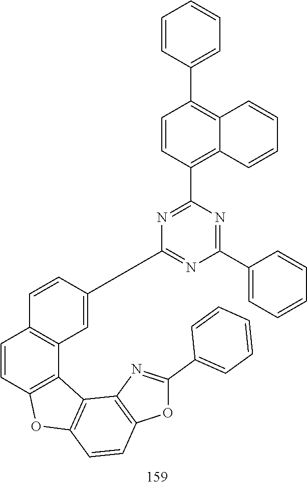 [SMILES: c1ccc(-c2nc(-c3ccc4ccc5oc6ccc7oc(-c8ccccc8)nc7c6c5c4c3)nc(-c3ccc(-c4ccccc4)c4ccccc34)n2)cc1] | 693.2 | 66 |
| 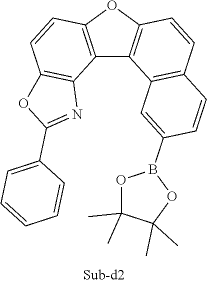 [SMILES: CC1(C)OB(c2ccc3ccc4oc5ccc6oc(-c7ccccc7)nc6c5c4c3c2)OC1(C)C] | 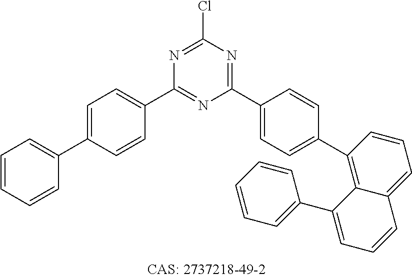 [SMILES: Clc1nc(-c2ccc(-c3ccccc3)cc2)nc(-c2ccc(-c3cccc4cccc(-c5ccccc5)c34)cc2)n1] | 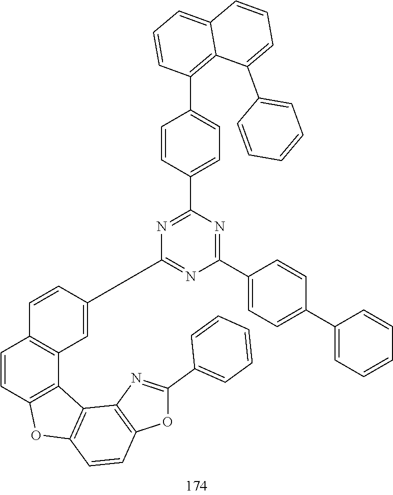 [SMILES: c1ccc(-c2ccc(-c3nc(-c4ccc(-c5cccc6cccc(-c7ccccc7)c56)cc4)nc(-c4ccc5ccc6oc7ccc8oc(-c9ccccc9)nc8c7c6c5c4)n3)cc2)cc1] | 845.3 | 60 |
| 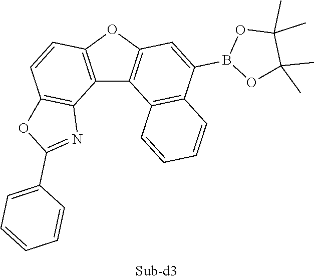 [SMILES: CC1(C)OB(c2cc3oc4ccc5oc(-c6ccccc6)nc5c4c3c3ccccc23)OC1(C)C] | 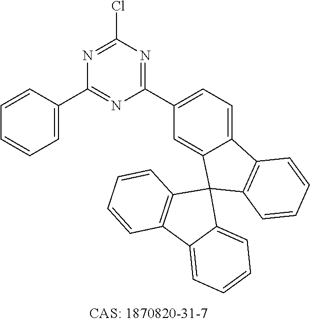 [SMILES: Clc1nc(-c2ccccc2)nc(-c2ccc3c(c2)C2(c4ccccc4-c4ccccc42)c2ccccc2-3)n1] | 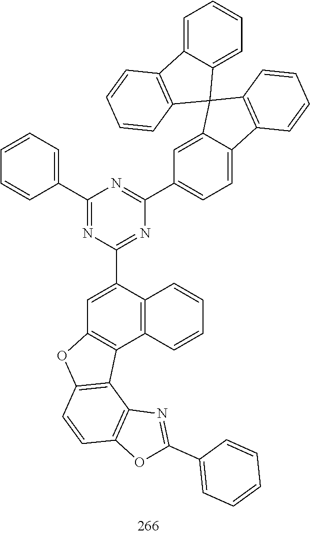 [SMILES: c1ccc(-c2nc(-c3ccc4c(c3)C3(c5ccccc5-c5ccccc53)c3ccccc3-4)nc(-c3cc4oc5ccc6oc(-c7ccccc7)nc6c5c4c4ccccc34)n2)cc1] | 805.3 | 61 |
| 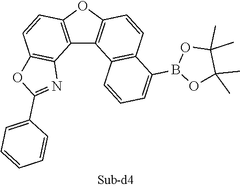 [SMILES: CC1(C)OB(c2cccc3c2ccc2oc4ccc5oc(-c6ccccc6)nc5c4c23)OC1(C)C] | 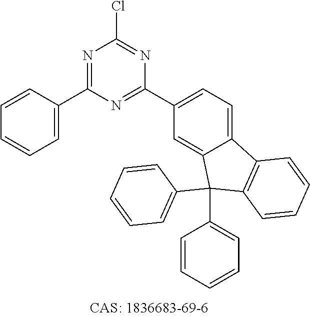 [SMILES: Clc1nc(-c2ccccc2)nc(-c2ccc3c(c2)C(c2ccccc2)(c2ccccc2)c2ccccc2-3)n1] | 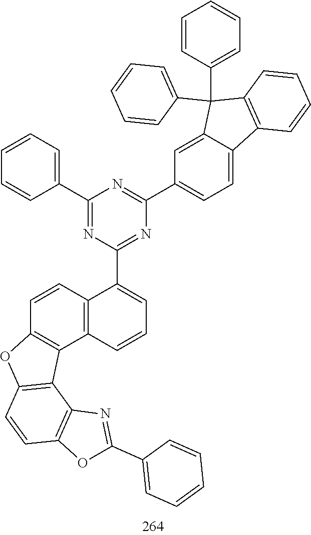 [SMILES: c1ccc(-c2nc(-c3ccc4c(c3)C(c3ccccc3)(c3ccccc3)c3ccccc3-4)nc(-c3cccc4c3ccc3oc5ccc6oc(-c7ccccc7)nc6c5c34)n2)cc1] | 807.3 | 62 |
| 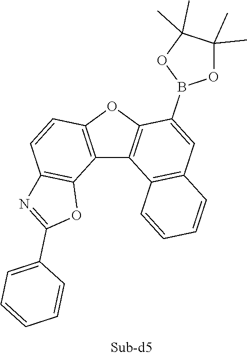 [SMILES: CC1(C)OB(c2cc3ccccc3c3c2oc2ccc4nc(-c5ccccc5)oc4c23)OC1(C)C] | 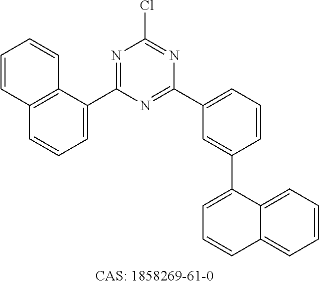 [SMILES: Clc1nc(-c2cccc(-c3cccc4ccccc34)c2)nc(-c2cccc3ccccc23)n1] | 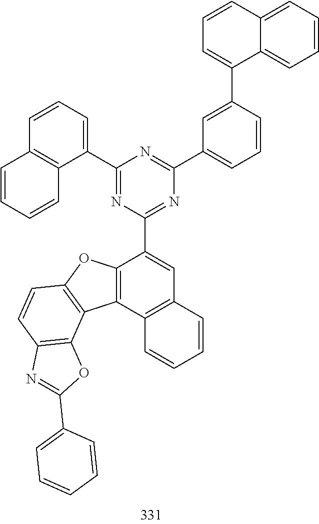 [SMILES: c1ccc(-c2nc3ccc4oc5c(-c6nc(-c7cccc(-c8cccc9ccccc89)c7)nc(-c7cccc8ccccc78)n6)cc6ccccc6c5c4c3o2)cc1] | 743.2 | 73 |
| 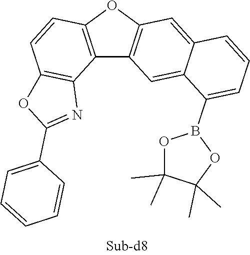 [SMILES: CC1(C)OB(c2cccc3cc4oc5ccc6oc(-c7ccccc7)nc6c5c4cc23)OC1(C)C] | 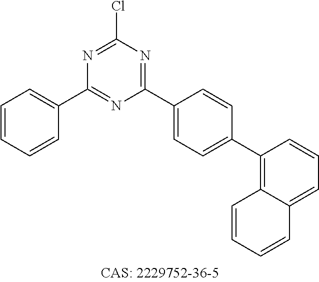 [SMILES: Clc1nc(-c2ccccc2)nc(-c2ccc(-c3cccc4ccccc34)cc2)n1] | 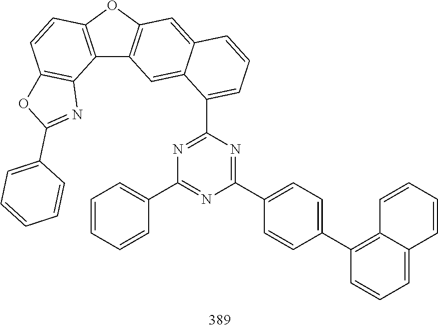 [SMILES: c1ccc(-c2nc(-c3ccc(-c4cccc5ccccc45)cc3)nc(-c3cccc4cc5oc6ccc7oc(-c8ccccc8)nc7c6c5cc34)n2)cc1] | 693.2 | 68 |
| 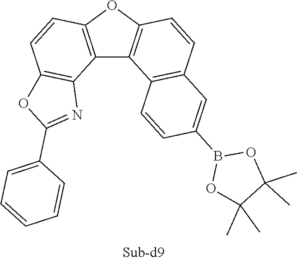 [SMILES: CC1(C)OB(c2ccc3c(ccc4oc5ccc6oc(-c7ccccc7)nc6c5c43)c2)OC1(C)C] | 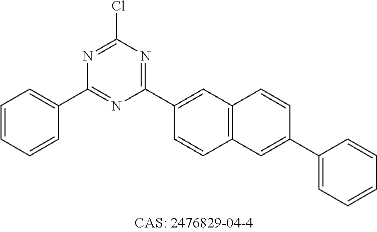 [SMILES: Clc1nc(-c2ccccc2)nc(-c2ccc3cc(-c4ccccc4)ccc3c2)n1] | 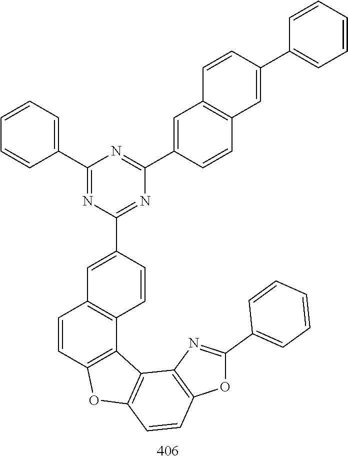 [SMILES: c1ccc(-c2ccc3cc(-c4nc(-c5ccccc5)nc(-c5ccc6c(ccc7oc8ccc9oc(-c%10ccccc%10)nc9c8c76)c5)n4)ccc3c2)cc1] | 693.2 | 72 |
| 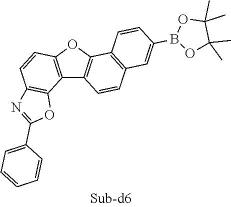 [SMILES: CC1(C)OB(c2ccc3c(ccc4c3oc3ccc5nc(-c6ccccc6)oc5c34)c2)OC1(C)C] | 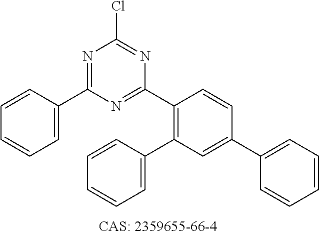 [SMILES: Clc1nc(-c2ccccc2)nc(-c2ccc(-c3ccccc3)cc2-c2ccccc2)n1] | 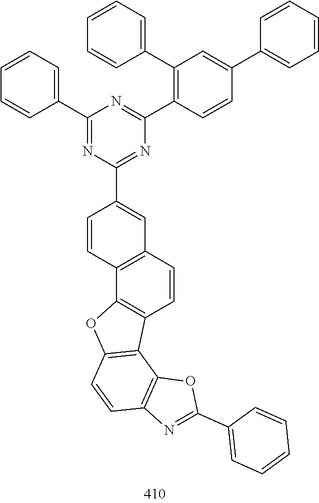 [SMILES: c1ccc(-c2ccc(-c3nc(-c4ccccc4)nc(-c4ccc5c(ccc6c5oc5ccc7nc(-c8ccccc8)oc7c56)c4)n3)c(-c3ccccc3)c2)cc1] | 719.2 | 66 |
| 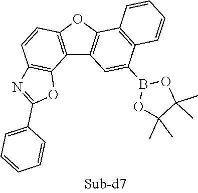 [SMILES: CC1(C)OB(c2cc3c(oc4ccc5nc(-c6ccccc6)oc5c43)c3ccccc23)OC1(C)C] | 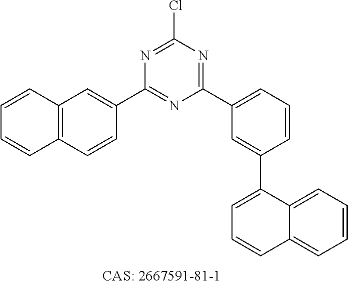 [SMILES: Clc1nc(-c2cccc(-c3cccc4ccccc34)c2)nc(-c2ccc3ccccc3c2)n1] | 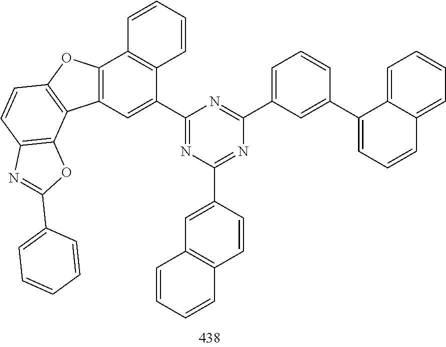 [SMILES: c1ccc(-c2nc3ccc4oc5c6ccccc6c(-c6nc(-c7cccc(-c8cccc9ccccc89)c7)nc(-c7ccc8ccccc8c7)n6)cc5c4c3o2)cc1] | 743.2 | 68 |
| 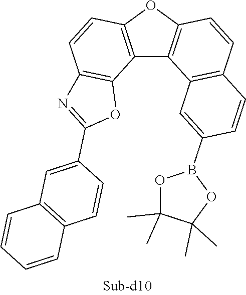 [SMILES: CC1(C)OB(c2ccc3ccc4oc5ccc6nc(-c7ccc8ccccc8c7)oc6c5c4c3c2)OC1(C)C] | 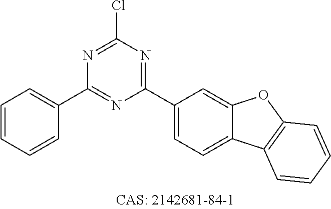 [SMILES: Clc1nc(-c2ccccc2)nc(-c2ccc3c(c2)oc2ccccc23)n1] | 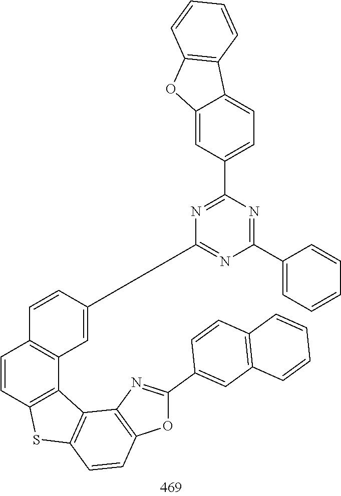 [SMILES: c1ccc(-c2nc(-c3ccc4c(c3)oc3ccccc34)nc(-c3ccc4ccc5sc6ccc7oc(-c8ccc9ccccc9c8)nc7c6c5c4c3)n2)cc1] | 707.2 | 69 |
| 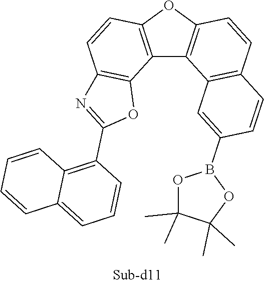 [SMILES: CC1(C)OB(c2ccc3ccc4oc5ccc6nc(-c7cccc8ccccc78)oc6c5c4c3c2)OC1(C)C] | 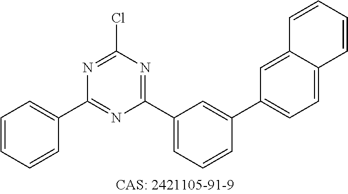 [SMILES: Clc1nc(-c2ccccc2)nc(-c2cccc(-c3ccc4ccccc4c3)c2)n1] | 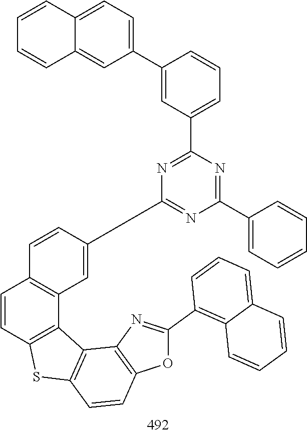 [SMILES: c1ccc(-c2nc(-c3cccc(-c4ccc5ccccc5c4)c3)nc(-c3ccc4ccc5sc6ccc7oc(-c8cccc9ccccc89)nc7c6c5c4c3)n2)cc1] | 743.2 | 70 |
| 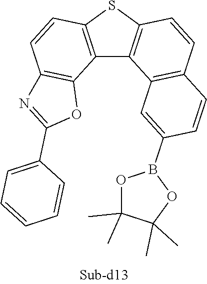 [SMILES: CC1(C)OB(c2ccc3ccc4sc5ccc6nc(-c7ccccc7)oc6c5c4c3c2)OC1(C)C] | 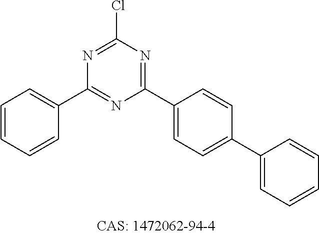 [SMILES: Clc1nc(-c2ccccc2)nc(-c2ccc(-c3ccccc3)cc2)n1] | 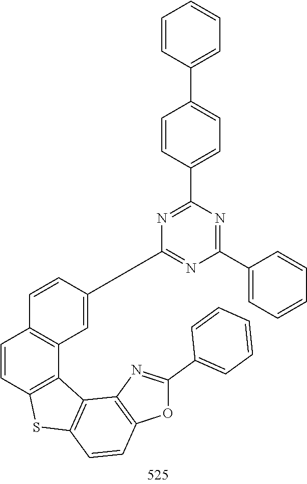 [SMILES: c1ccc(-c2ccc(-c3nc(-c4ccccc4)nc(-c4ccc5ccc6sc7ccc8oc(-c9ccccc9)nc8c7c6c5c4)n3)cc2)cc1] | 659.2 | 69 |
| 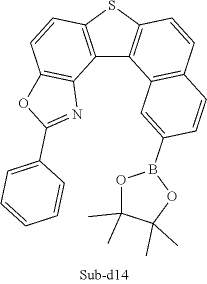 [SMILES: CC1(C)OB(c2ccc3ccc4sc5ccc6oc(-c7ccccc7)nc6c5c4c3c2)OC1(C)C] | 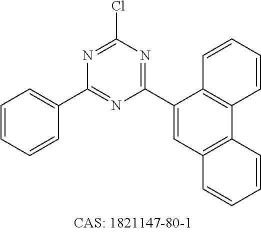 [SMILES: Clc1nc(-c2ccccc2)nc(-c2cc3ccccc3c3ccccc23)n1] | 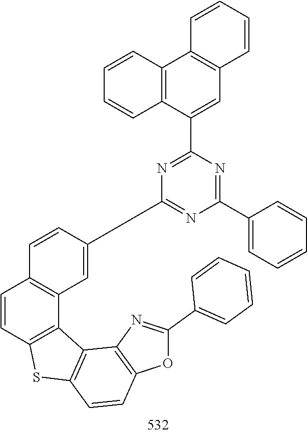 [SMILES: c1ccc(-c2nc(-c3ccc4ccc5sc6ccc7oc(-c8ccccc8)nc7c6c5c4c3)nc(-c3cc4ccccc4c4ccccc34)n2)cc1] | 683.2 | 70 |
| 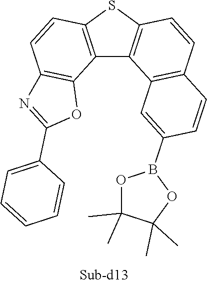 [SMILES: CC1(C)OB(c2ccc3ccc4sc5ccc6nc(-c7ccccc7)oc6c5c4c3c2)OC1(C)C] | 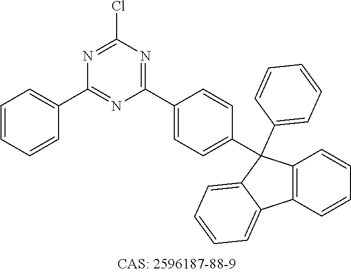 [SMILES: Clc1nc(-c2ccccc2)nc(-c2ccc(C3(c4ccccc4)c4ccccc4-c4ccccc43)cc2)n1] | 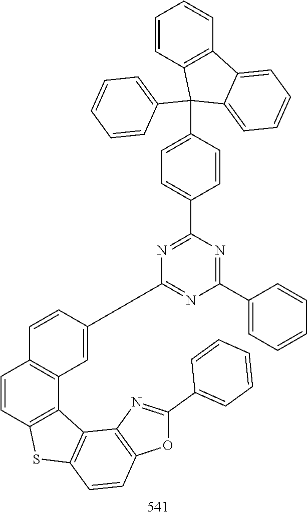 [SMILES: c1ccc(-c2nc(-c3ccc(C4(c5ccccc5)c5ccccc5-c5ccccc54)cc3)nc(-c3ccc4ccc5sc6ccc7oc(-c8ccccc8)nc7c6c5c4c3)n2)cc1] | 823.3 | 62 |
| 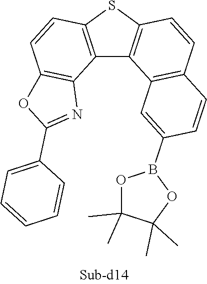 [SMILES: CC1(C)OB(c2ccc3ccc4sc5ccc6oc(-c7ccccc7)nc6c5c4c3c2)OC1(C)C] | 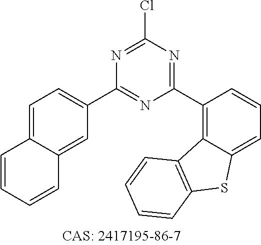 [SMILES: Clc1nc(-c2ccc3ccccc3c2)nc(-c2cccc3sc4ccccc4c23)n1] | 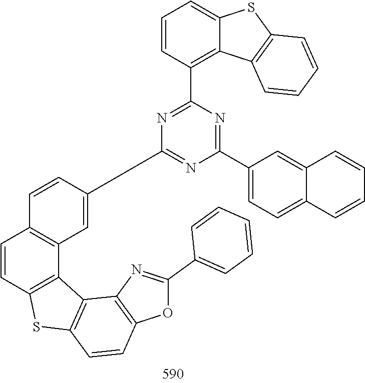 [SMILES: c1ccc(-c2nc3c(ccc4sc5ccc6ccc(-c7nc(-c8ccc9ccccc9c8)nc(-c8cccc9sc%10ccccc%10c89)n7)cc6c5c43)o2)cc1] | 739.2 | 67 |
| 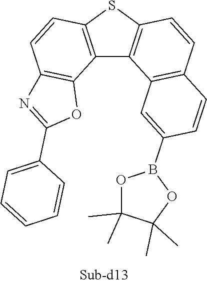 [SMILES: CC1(C)OB(c2ccc3ccc4sc5ccc6nc(-c7ccccc7)oc6c5c4c3c2)OC1(C)C] | 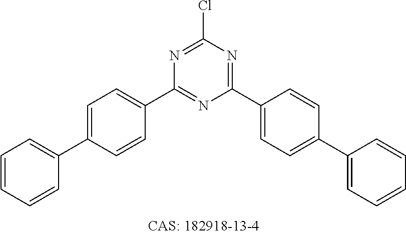 [SMILES: Clc1nc(-c2ccc(-c3ccccc3)cc2)nc(-c2ccc(-c3ccccc3)cc2)n1] | 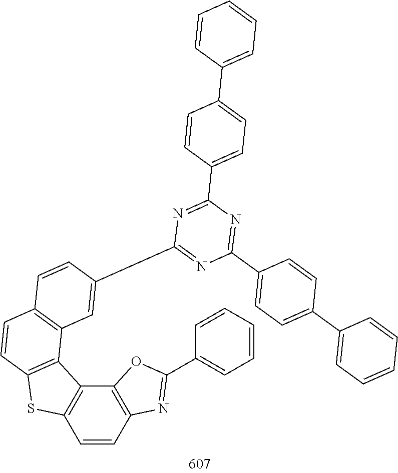 [SMILES: c1ccc(-c2ccc(-c3nc(-c4ccc(-c5ccccc5)cc4)nc(-c4ccc5ccc6sc7ccc8nc(-c9ccccc9)oc8c7c6c5c4)n3)cc2)cc1] | 735.2 | 72 |
| 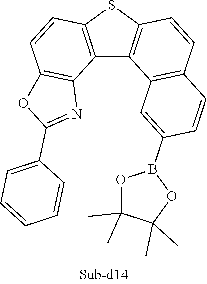 [SMILES: CC1(C)OB(c2ccc3ccc4sc5ccc6oc(-c7ccccc7)nc6c5c4c3c2)OC1(C)C] | 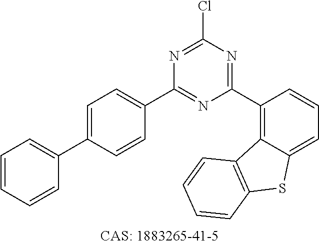 [SMILES: Clc1nc(-c2ccc(-c3ccccc3)cc2)nc(-c2cccc3sc4ccccc4c23)n1] | 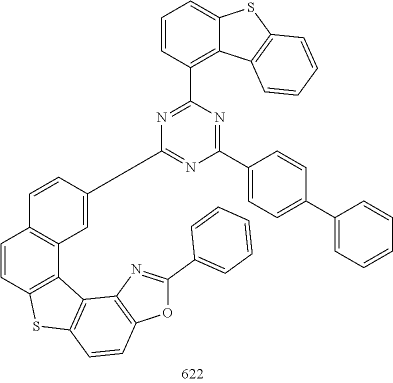 [SMILES: c1ccc(-c2ccc(-c3nc(-c4ccc5ccc6sc7ccc8oc(-c9ccccc9)nc8c7c6c5c4)nc(-c4cccc5sc6ccccc6c45)n3)cc2)cc1] | 765.2 | 69 |
| 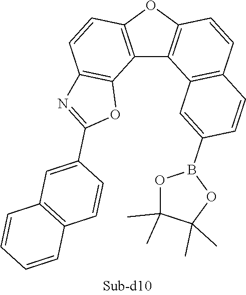 [SMILES: CC1(C)OB(c2ccc3ccc4oc5ccc6nc(-c7ccc8ccccc8c7)oc6c5c4c3c2)OC1(C)C] | 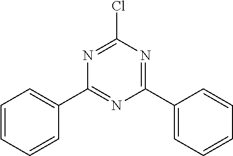 [SMILES: Clc1nc(-c2ccccc2)nc(-c2ccccc2)n1] | 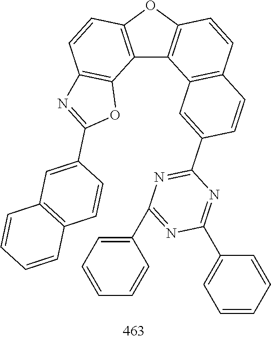 [SMILES: c1ccc(-c2nc(-c3ccccc3)nc(-c3ccc4ccc5oc6ccc7nc(-c8ccc9ccccc9c8)oc7c6c5c4c3)n2)cc1] | 617.2 | 58 |
| 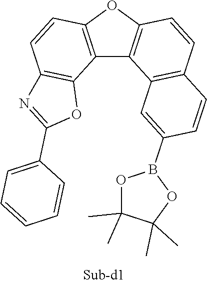 [SMILES: CC1(C)OB(c2ccc3ccc4oc5ccc6nc(-c7ccccc7)oc6c5c4c3c2)OC1(C)C] | 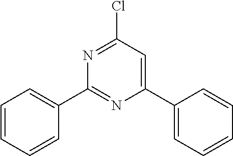 [SMILES: Clc1cc(-c2ccccc2)nc(-c2ccccc2)n1] | 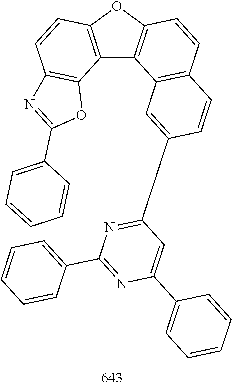 [SMILES: c1ccc(-c2cc(-c3ccc4ccc5oc6ccc7nc(-c8ccccc8)oc7c6c5c4c3)nc(-c3ccccc3)n2)cc1] | 566.2 | 60 |

NMR data of some compounds:
NMR data of Compound 6: ¹H-NMR (400 MHz, Methylene-Chloride-D2) δ ppm 9.41 (s, 1H), 8.82 (d, 2H), 8.65 (d, 1H), 8.36-8.33 (m, 3H), 8.29-8.23 (m, 2H), 8.11 (d, 1H), 8.06 (d, 1H), 7.77-7.48 (m ,12H), 7.37 (t, 1H).
NMR data of Compound 389: ¹H-NMR (400 MHz, Methylene-Chloride-D2) δ ppm 9.55 (s, 1H), 8.82 (d, 2H), 8.65 (d, 1H), 8.38-8.34 (m, 3H), 8.28 (d, 2H), 8.08 (d, 1H), 7.98 (d, 2H), 7.88 (d, 1H), 7.79-7.70 (m, 3H), 7.67-7.51 (m, 8H), 7.47 (t, 1H), 7.42-7.33 (m, 3H).

### Fabrication and Evaluation of Organic Electroluminescent Devices

### Example 1: Preparation of a Red Light Organic Electroluminescent Device

First, anode pretreatment was performed by the following processes. A surface of an ITO/Ag/ITO substrate, with thicknesses of ITO/Ag/ITO being 100 Å, 1000 Å, and 100 Å, respectively, was treated using ultraviolet ozone and O₂:N₂ plasma to increase the work function of the anode, and then cleaned with an organic solvent to remove impurities and oil on the ITO substrate.

Compound PD was deposited by vacuum evaporation on the experimental substrate (anode) to form a hole injection layer (HIL) having a thickness of 100 Å, and then α-NPD was deposited by vacuum evaporation on the hole injection layer to form a first hole transport layer having a thickness of 1080 Å.

Compound HT-1 was deposited by vacuum evaporation on the first hole transport layer to form a second hole transport layer having a thickness of 890 Å.

Next, Compound 1, Compound RH-P, and Compound RD-1 were co-deposited by evaporation on the second hole transport layer at an evaporation rate ratio of 49%:49%:2% to form a red light organic light emitting layer (EML) having a thickness of 425 Å.

Compound ET-1 and Compound LiQ were mixed at a 1:1 weight ratio and deposited by evaporation on the organic light emitting layer to form an electron transport layer (ETL) having a thickness of 350 Å; Yb was deposited by evaporation on the electron transport layer to form an electron injection layer (EIL) having a thickness of 10 Å; and then magnesium (Mg) and silver (Ag) were mixed at an evaporation rate ratio of 1:9, and deposited by vacuum evaporation on the electron injection layer to form a cathode having a thickness of 130 Å.

Further, Compound CP-1 was deposited by vacuum evaporation on the above cathode to form a capping layer (CPL) having a thickness of 800 Å, completing the fabrication of a red light organic electroluminescent device.

### Examples 2 to 40

Organic electroluminescent devices were fabricated respectively by the same method as used in Example 1, except that Compound 1 in Example 1 was replaced with a corresponding Compound X shown in the following Table 7 when an organic light emitting layer was formed.

### Comparative Examples 1 to 3

Organic electroluminescent devices were fabricated respectively by the same method as used in Example 1, except that Compound 1 in Example 1 was replaced with a corresponding one of Compound A, Compound B, Compound C when an organic light emitting layer was formed.

Structures of the main materials used in the Examples and Comparative Examples are as follows:

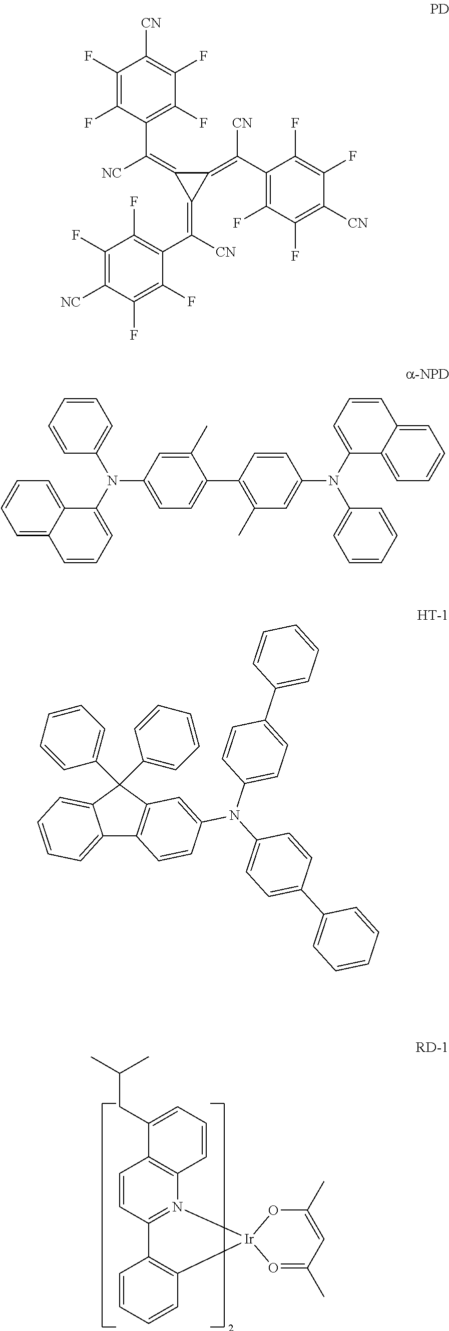

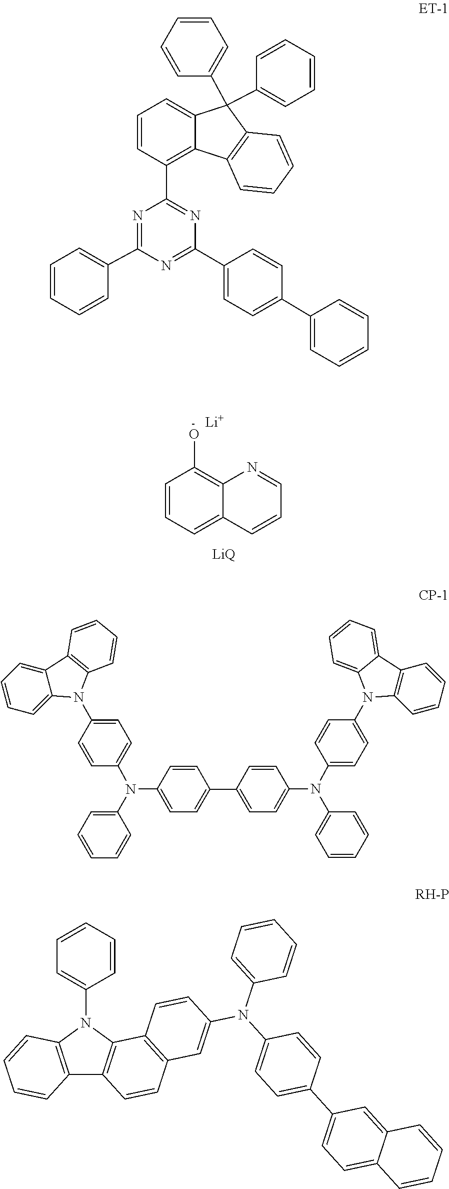

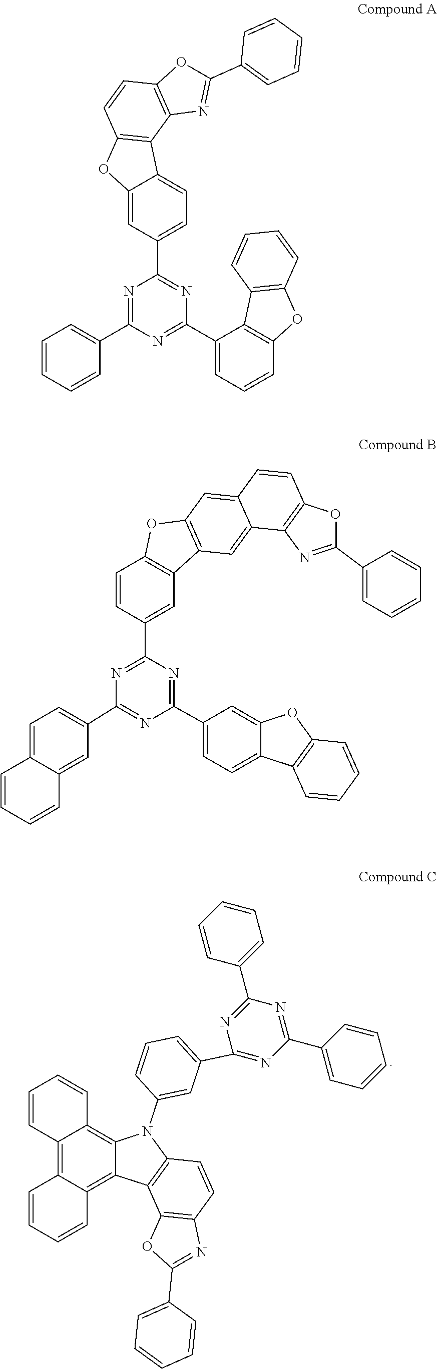 [SMILES: c1ccc(-c2nc(-c3ccc4c(c3)oc3ccc5oc(-c6ccccc6)nc5c34)nc(-c3cccc4oc5ccccc5c34)n2)cc1 ; c1ccc(-c2nc3c(ccc4cc5oc6ccc(-c7nc(-c8ccc9ccccc9c8)nc(-c8ccc9c(c8)oc8ccccc89)n7)cc6c5cc43)o2)cc1 ; c1ccc(-c2nc(-c3ccccc3)nc(-c3cccc(-n4c5ccc6nc(-c7ccccc7)oc6c5c5c6ccccc6c6ccccc6c54)c3)n2)cc1]

The red light organic electroluminescent devices fabricated in Examples 1 to 40 and Comparative Examples 1 to 3 were tested for their performance. Specifically, the IVL characteristics of the devices were tested under the condition of 10 mA/cm², and the T95 lifetime of the devices was tested under the condition of 20 mA/cm². Test results are shown in Table 7.

TABLE 7

| | | Operating | Luminescence | | | T95(h) |
|---|---|---|---|---|---|---|
| Example No. | Compound X | voltage (V) | efficiency Cd/A | CIEx | CIEy | @20 mA/cm² |
| Example 1 | Compound 1 | 3.40 | 65.8 | 0.680 | 0.320 | 568 |
| Example 2 | Compound 6 | 3.44 | 64.5 | 0.680 | 0.320 | 520 |
| Example 3 | Compound 8 | 3.42 | 65.6 | 0.680 | 0.320 | 518 |
| Example 4 | Compound 12 | 3.42 | 66.5 | 0.680 | 0.320 | 549 |
| Example 5 | Compound 19 | 3.39 | 65.6 | 0.680 | 0.320 | 526 |
| Example 6 | Compound 24 | 3.40 | 64.2 | 0.680 | 0.320 | 535 |
| Example 7 | Compound 26 | 3.44 | 66.1 | 0.680 | 0.320 | 531 |
| Example 8 | Compound 27 | 3.43 | 64.1 | 0.680 | 0.320 | 535 |
| Example 9 | Compound 30 | 3.44 | 65.6 | 0.680 | 0.320 | 534 |
| Example 10 | Compound 32 | 3.43 | 66.7 | 0.680 | 0.320 | 532 |
| Example 11 | Compound 35 | 3.42 | 66.8 | 0.680 | 0.320 | 536 |
| Example 12 | Compound 42 | 3.39 | 64.0 | 0.680 | 0.320 | 517 |
| Example 13 | Compound 50 | 3.42 | 65.2 | 0.680 | 0.320 | 564 |
| Example 14 | Compound 60 | 3.39 | 66.5 | 0.680 | 0.320 | 543 |
| Example 15 | Compound 66 | 3.39 | 64.5 | 0.680 | 0.320 | 522 |
| Example 16 | Compound 70 | 3.40 | 64.2 | 0.680 | 0.320 | 530 |
| Example 17 | Compound 82 | 3.39 | 65.6 | 0.680 | 0.320 | 528 |
| Example 18 | Compound 88 | 3.42 | 64.1 | 0.680 | 0.320 | 547 |
| Example 19 | Compound 93 | 3.44 | 66.3 | 0.680 | 0.320 | 539 |
| Example 20 | Compound 112 | 3.39 | 64.9 | 0.680 | 0.320 | 519 |
| Example 21 | Compound 135 | 3.39 | 65.1 | 0.680 | 0.320 | 524 |
| Example 22 | Compound 159 | 3.42 | 65.3 | 0.680 | 0.320 | 528 |
| Example 23 | Compound 174 | 3.39 | 65.8 | 0.680 | 0.320 | 519 |
| Example 24 | Compound 266 | 3.44 | 60.1 | 0.680 | 0.320 | 471 |
| Example 25 | Compound 264 | 3.44 | 61.1 | 0.680 | 0.320 | 472 |
| Example 26 | Compound 331 | 3.39 | 61.9 | 0.680 | 0.320 | 469 |
| Example 27 | Compound 389 | 3.40 | 61.1 | 0.680 | 0.320 | 480 |
| Example 28 | Compound 406 | 3.39 | 60.8 | 0.680 | 0.320 | 480 |
| Example 29 | Compound 410 | 3.39 | 61.5 | 0.680 | 0.320 | 477 |
| Example 30 | Compound 438 | 3.44 | 61.4 | 0.680 | 0.320 | 467 |
| Example 31 | Compound 469 | 3.42 | 65.3 | 0.680 | 0.320 | 523 |
| Example 32 | Compound 492 | 3.44 | 65.1 | 0.680 | 0.320 | 529 |
| Example 33 | Compound 525 | 3.42 | 60.8 | 0.680 | 0.320 | 517 |
| Example 34 | Compound 532 | 3.44 | 61.4 | 0.680 | 0.320 | 522 |
| Example 35 | Compound 541 | 3.44 | 60.7 | 0.680 | 0.320 | 531 |
| Example 36 | Compound 590 | 3.39 | 60.6 | 0.680 | 0.320 | 521 |
| Example 37 | Compound 607 | 3.40 | 60.7 | 0.680 | 0.320 | 539 |
| Example 38 | Compound 622 | 3.40 | 62.0 | 0.680 | 0.320 | 528 |
| Example 39 | Compound 463 | 3.43 | 66.3 | 0.680 | 0.320 | 535 |
| Example 40 | Compound 643 | 3.39 | 66.0 | 0.680 | 0.320 | 532 |
| Comparative | Compound A | 3.48 | 53.5 | 0.68 | 0.32 | 401 |
| Example 1 | | | | | | |
| Comparative | Compound B | 3.49 | 52.3 | 0.68 | 0.32 | 422 |
| Example 2 | | | | | | |
| Comparative | Compound C | 3.46 | 49.5 | 0.68 | 0.32 | 365 |
| Example 3 | | | | | | |

As can be seen from Table 7 above, the use of the compounds of the present application as the host material of the red light organic electroluminescent device improves the efficiency of the devices by at least 12.3%, and prolongs the service life thereof by at least 10.7%. This is because, the structure of the compound of the present application comprises naphthafurooxazolyl/thiazolyl and a electron-deficient heteroaryl such as triazinyl and pyrimidyl. The naphthafuryl group and the oxazolyl/thiazolyl group both have electron transport properties; and after these two groups are fused together, the conjugated system is increased, which enhances the electron transport performance of the groups. The triazinyl or pyrimidyl group has an excellent electron transport property. Linking of the naphthafurooxazolyl/thiazolyl group with the electron-deficient heteroaryl group such as triazinyl and pyrimidyl gives the compounds of the present application excellent electron transport properties. The compounds of the present application, when mixed with a hole transport material to form a mixed-type host material, can improve the balance of charge carriers in an light emitting layer, expand a region where the carriers recombine, improve the generation and utilization efficiency of excitons as well as the luminescence efficiency and the service life of a device.

1. A nitrogen-containing compound having a structure as shown in Formula 1: 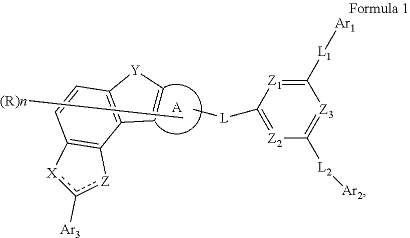
wherein, Y is selected from S or O;
one of X and Z is —N═, and the other is O or S;
Z₁, Z₂, and Z₃ are selected from C(R₁) or N, and at least two of Z₁, Z₂, and Z₃ are N;
Ring A is selected from a naphthalene ring or a phenanthrene ring;
L, L₁, and L₂ are identical or different, and are each independently selected from a single bond, a substituted or unsubstituted arylene having 6 to 30 carbon atoms, or a substituted or unsubstituted heteroarylene having 3 to 30 carbon atoms;
Ar₁, Ar₂, and Ar₃ are identical or different, and are each independently selected from a substituted or unsubstituted aryl having 6 to 40 carbon atoms, a substituted or unsubstituted heteroaryl having 3 to 40 carbon atoms, an alkyl having 1 to 10 carbon atoms, or a cycloalkyl having 3 to 10 carbon atoms;
substituents of L, L₁, L₂, Ar₁, Ar₂, and Ar₃ are identical or different, and are each independently selected from deuterium, a cyano, a halogen, an alkyl having 1 to 10 carbon atoms, a haloalkyl having 1 to 10 carbon atoms, a deuterated alkyl having 1 to 10 carbon atoms, a trialkylsiyl having 3 to 12 carbon atoms, a triphenylsilyl, an aryl having 6 to 20 carbon atoms, a deuterated aryl having 6 to 20 carbon atoms, a heteroaryl having 3 to 20 carbon atoms, a cycloalkyl having 3 to 10 carbon atoms, an alkoxy having 1 to 10 carbon atoms, an alkylthio having 1 to 10 carbon atoms, an aryloxy having 6 to 20 carbon atoms, or an arylthio having 6 to 20 carbon atoms; optionally, any two adjacent substituents form a saturated or unsaturated 3-membered to 15-membered ring;
each R₁ and each R are identical or different, and are each independently selected from hydrogen, deuterium, a cyano, a halogen, an alkyl having 1 to 10 carbon atoms, a haloalkyl having 1 to 10 carbon atoms, a deuterated alkyl having 1 to 10 carbon atoms, a trialkylsiyl having 3 to 12 carbon atoms, a triphenylsilyl, an aryl having 6 to 20 carbon atoms, a heteroaryl having 3 to 20 carbon atoms, or a cycloalkyl having 3 to 10 carbon atoms; optionally, any two adjacent R₁s or any two adjacent Rs form a ring; n is selected from 1, 2, 3, 4, 5, 6, 7, 8, or 9.

2. The nitrogen-containing compound according to claim 1, wherein the nitrogen-containing compound is selected from structures shown in the following Formulae (1-1) to (1-16):

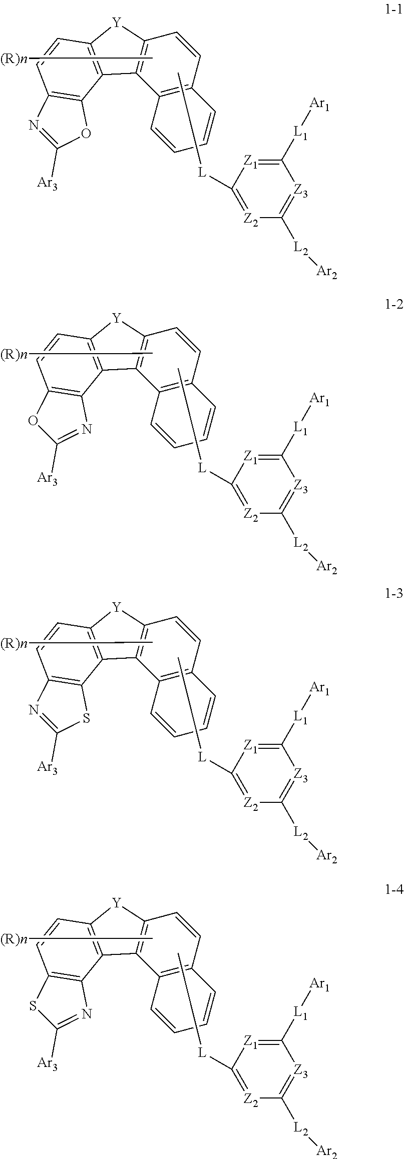

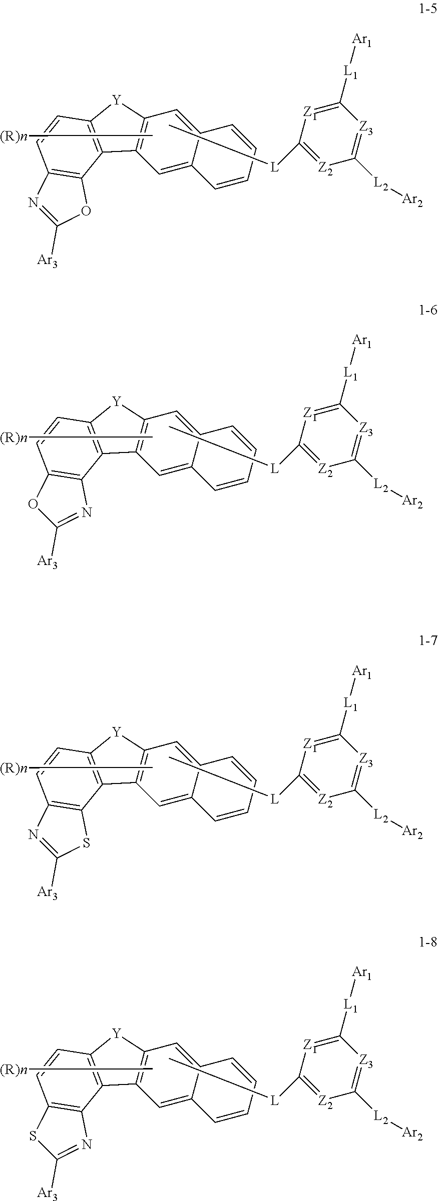

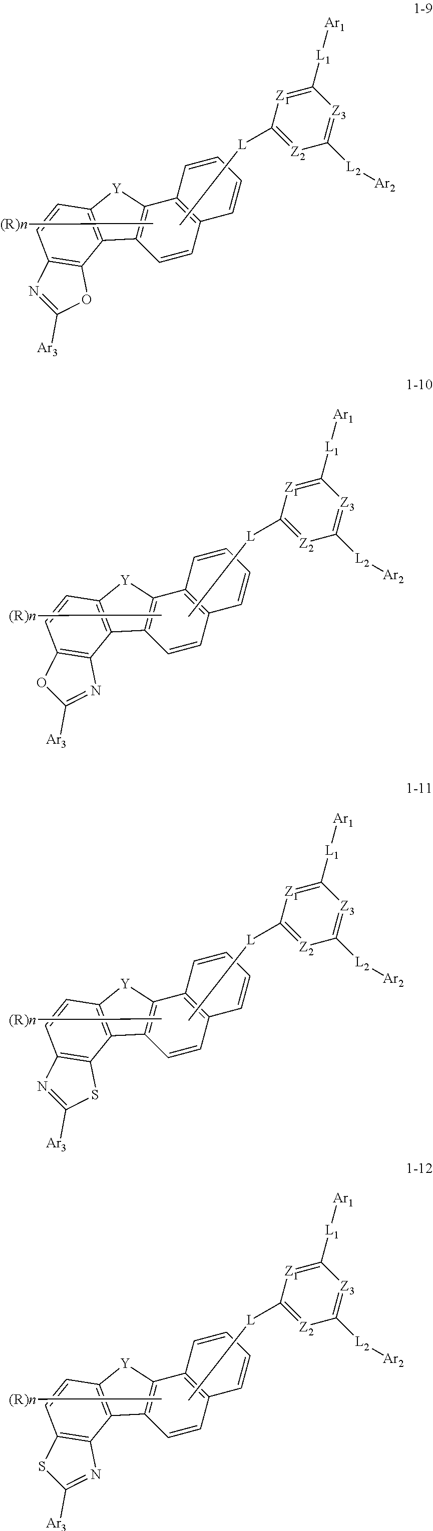

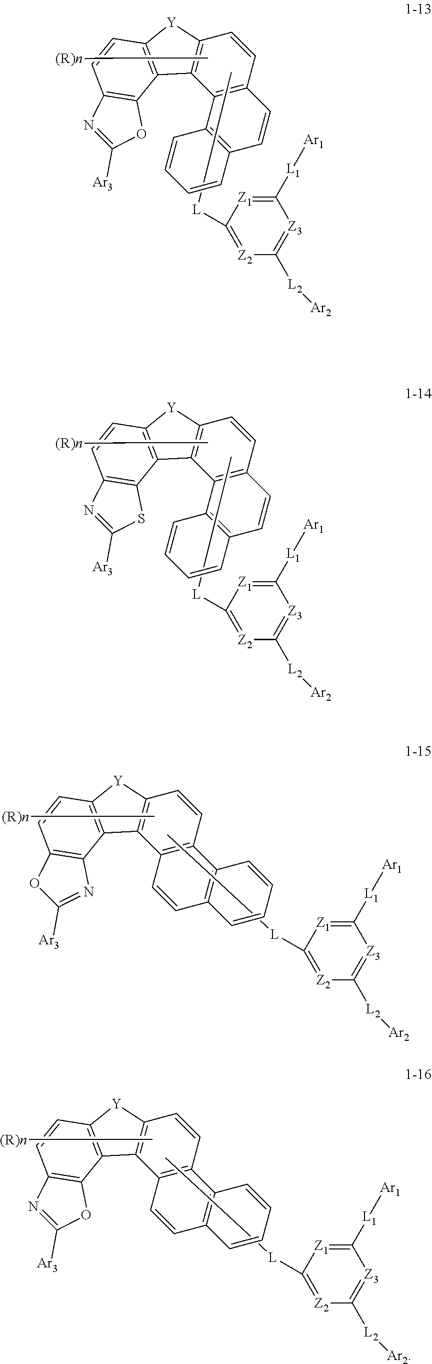

3. The nitrogen-containing compound according to claim 1, wherein Z₁ and Z₃ are N, and Z₂ is selected from C(H) or N;
or, Z₁ and Z₂ are N, and Z₃ is selected from C(H) or N;
or, Z₁, Z₂, and Z₃ are all N.

4. The nitrogen-containing compound according to claim 1, wherein Ar₁, Ar₂, and Ar₃ are identical or different, and are each independently selected from a substituted or unsubstituted aryl having 6 to 25 carbon atoms, or a substituted or unsubstituted heteroaryl having 5 to 20 carbon atoms;
optionally, the substituents of Ar₁, Ar₂, and Ar₃ are each independently selected from deuterium, a halogen, a cyano, a haloalkyl having 1 to 4 carbon atoms, a deuterated alkyl having 1 to 4 carbon atoms, an alkyl having 1 to 4 carbon atoms, a cycloalkyl having 5 to 10 carbon atoms, an aryl having 6 to 15 carbon atoms, a heteroaryl having 5 to 12 carbon atoms, a trialkylsilyl having 3 to 8 carbon atoms, or a deuterated aryl having 6 to 15 carbon atoms; optionally, any two adjacent substituents form a benzene ring or a fluorene ring.

5. The nitrogen-containing compound according to claim 1, wherein Ar₁, Ar₂, and Ar₃ are each independently selected from a substituted or unsubstituted group W; the unsubstituted group W is selected from the group consisting of the following groups: 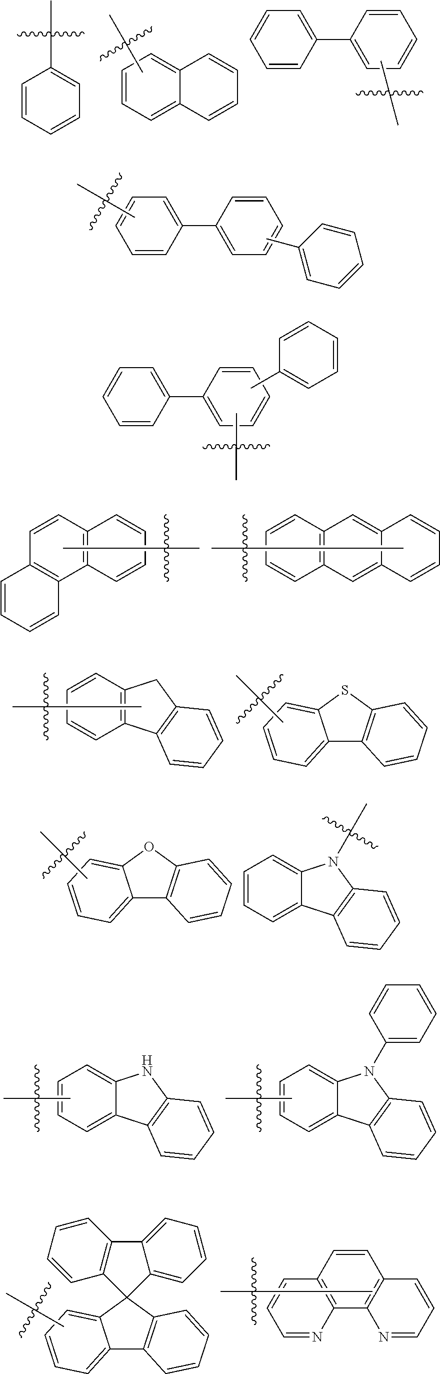 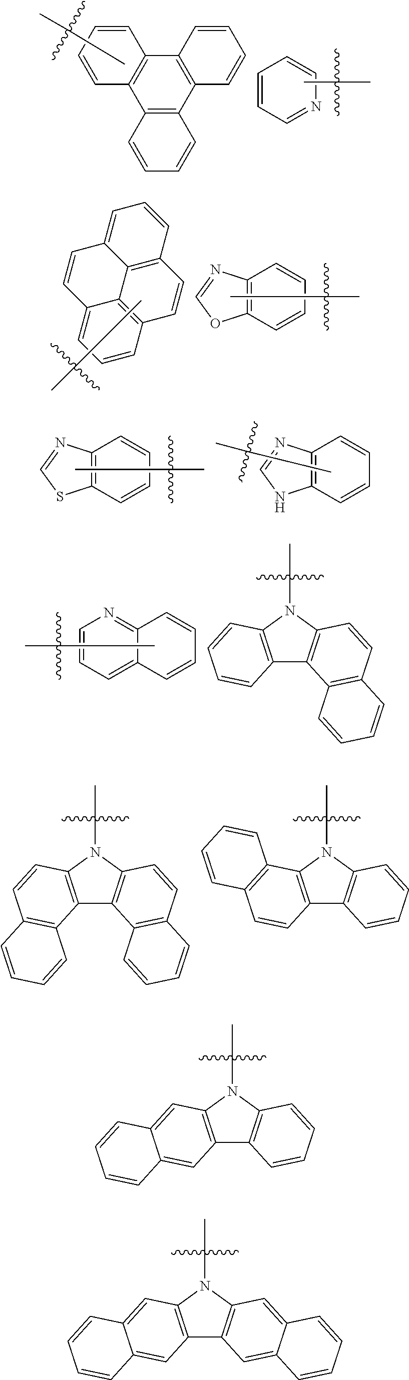 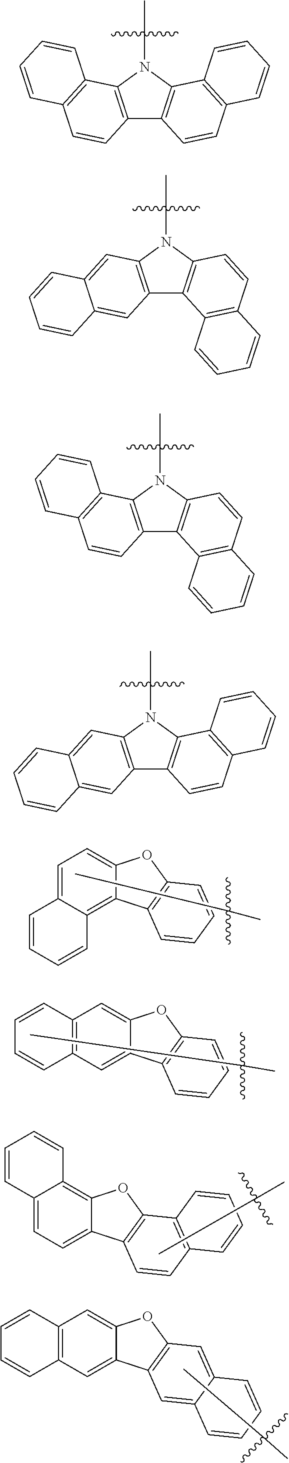 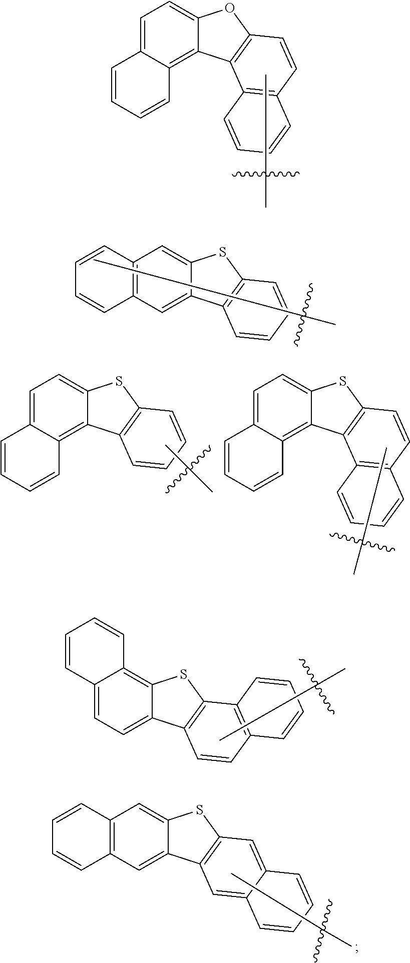
the substituted group W each have one or more than two substituents, the substituents of the substituted group W being each independently selected from deuterium, a fluorine, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, a dibenzofuranyl, a dibenzothiophenyl, a carbazolyl, a benzoxazolyl, or a benzothiazolyl; when the number of the substituents of the group W is greater than 1, the substituents are identical or different.

6. The nitrogen-containing compound according to claim 1, wherein L, L₁, and L₂ are identical or different, and are each independently selected from a single bond, a substituted or unsubstituted arylene having 6 to 15 carbon atoms, or a substituted or unsubstituted heteroarylene having 5 to 18 carbon atoms;
optionally, the substituents of L, L₁, and L₂ are each independently selected from deuterium, a fluorine, a cyano, an alkyl having 1 to 5 carbon atoms, a trialkylsilyl having 3 to 8 carbon atoms, a fluoroalkyl having 1 to 4 carbon atoms, a deuterated alkyl having 1 to 4 carbon atoms, a phenyl, or a naphthyl.

7. The nitrogen-containing compound according to claim 1, wherein L, L₁, and L₂ are each independently selected from a single bond or a substituted or unsubstituted group Q; the unsubstituted group Q is selected from the following groups: 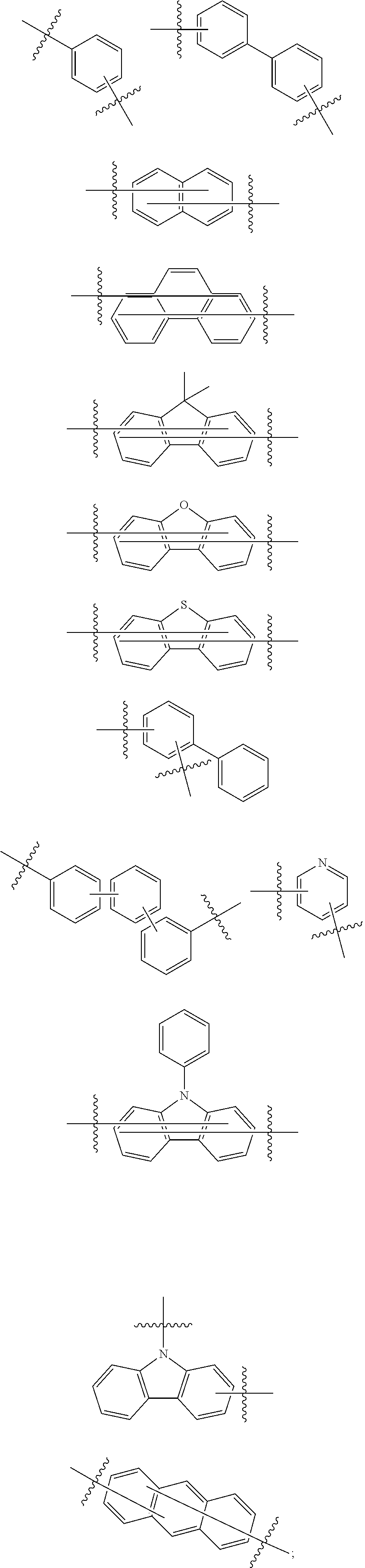
the substituted group Q each have one or more than two substituents, the substituents of the substituted group Q are each independently selected from deuterium, a fluorine, a cyano, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, a dibenzofuranyl, a dibenzothiophenyl, or a carbazolyl; when the number of the substituents of the group Q is greater than 1, the substituents are identical or different.

8. The nitrogen-containing compound according to claim 1, wherein Ar₁ and Ar₂ are each independently selected from the group consisting of the following groups: 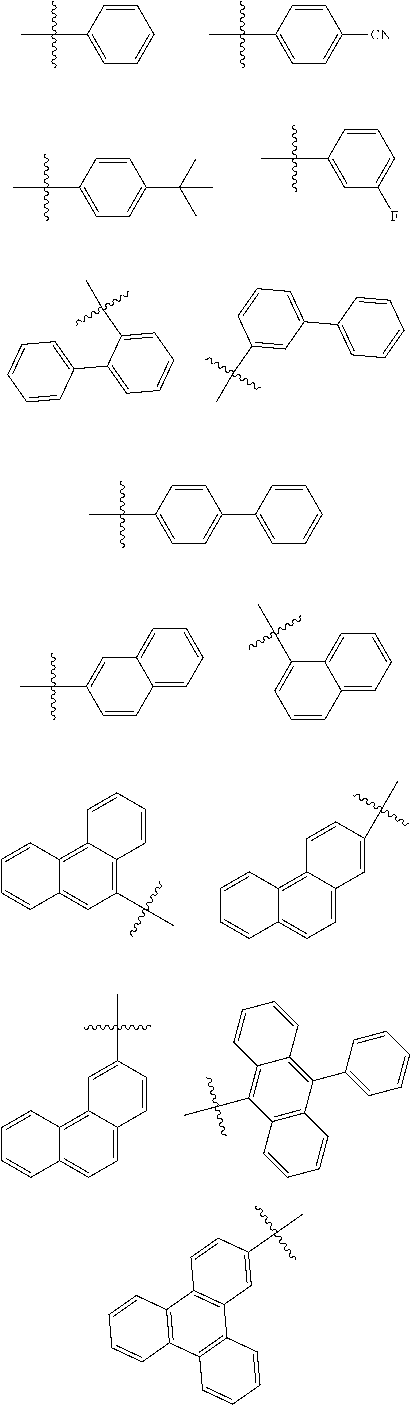 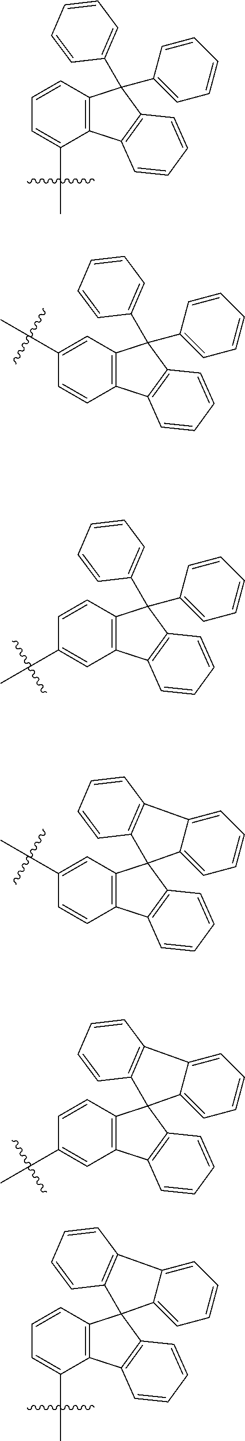 [SMILES: *c1cccc2c1-c1ccccc1C2(c1ccccc1)c1ccccc1 ; *c1ccc2c(c1)C(c1ccccc1)(c1ccccc1)c1ccccc1-2 ; *c1ccc2c(c1)-c1ccccc1C2(c1ccccc1)c1ccccc1 ; *c1ccc2c(c1)C1(c3ccccc3-c3ccccc31)c1ccccc1-2 ; *c1ccc2c(c1)-c1ccccc1C21c2ccccc2-c2ccccc21 ; *c1cccc2c1-c1ccccc1C21c2ccccc2-c2ccccc21] 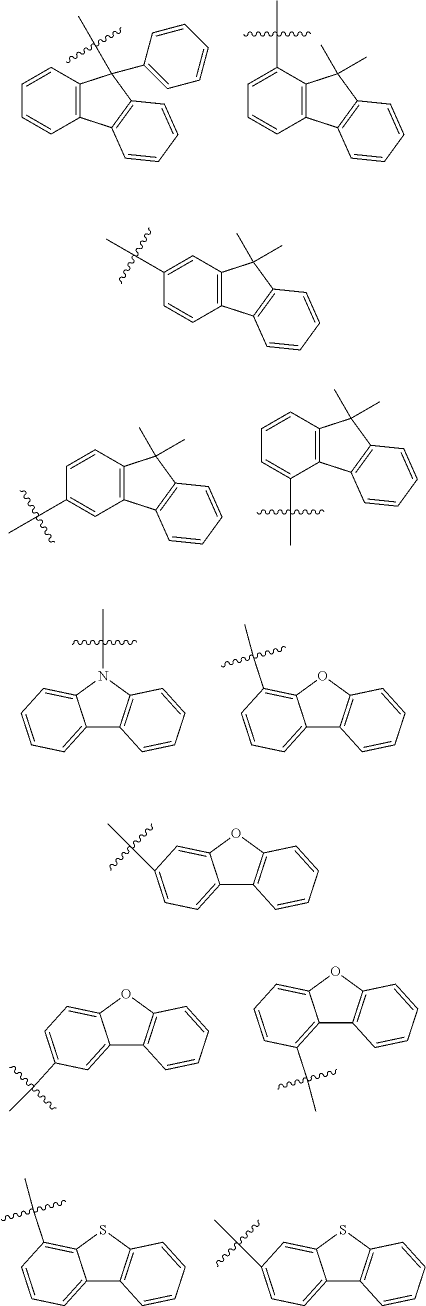 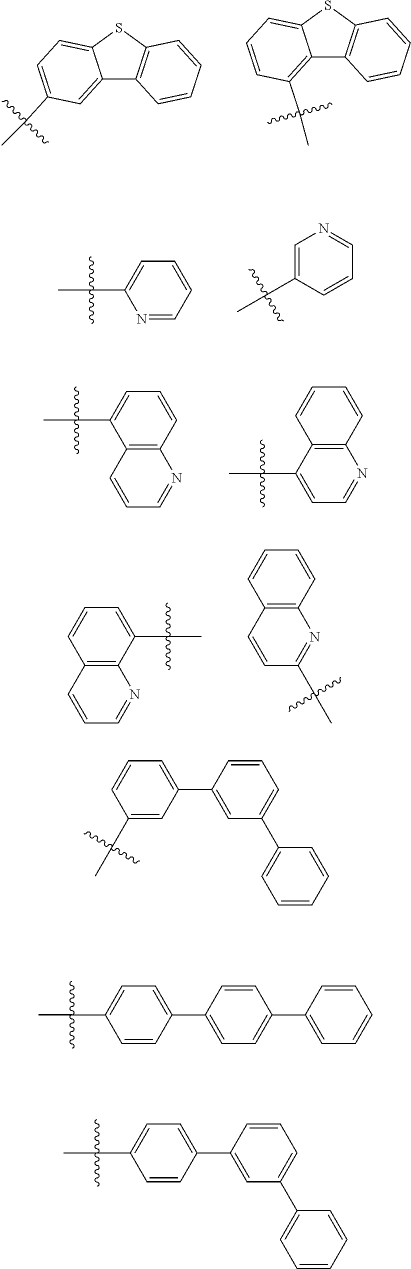 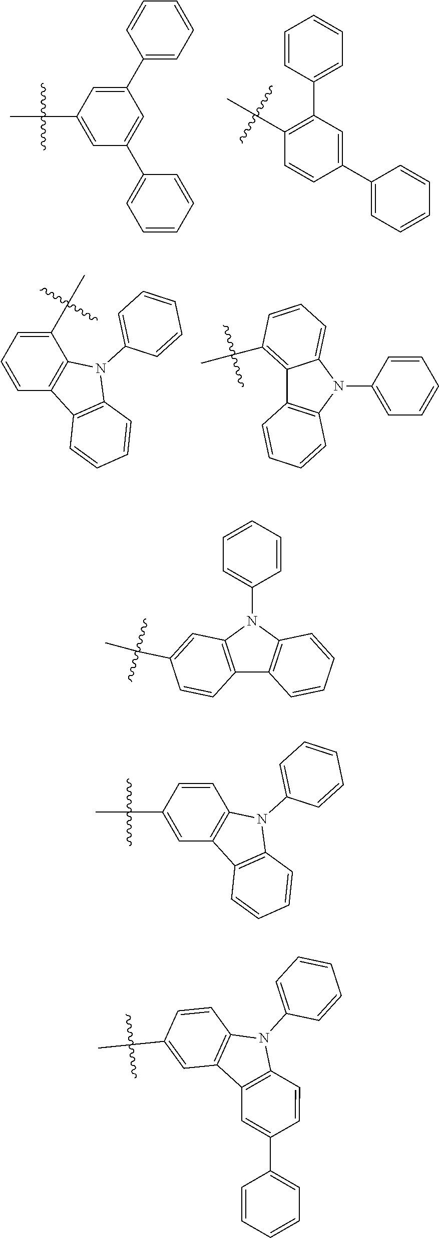 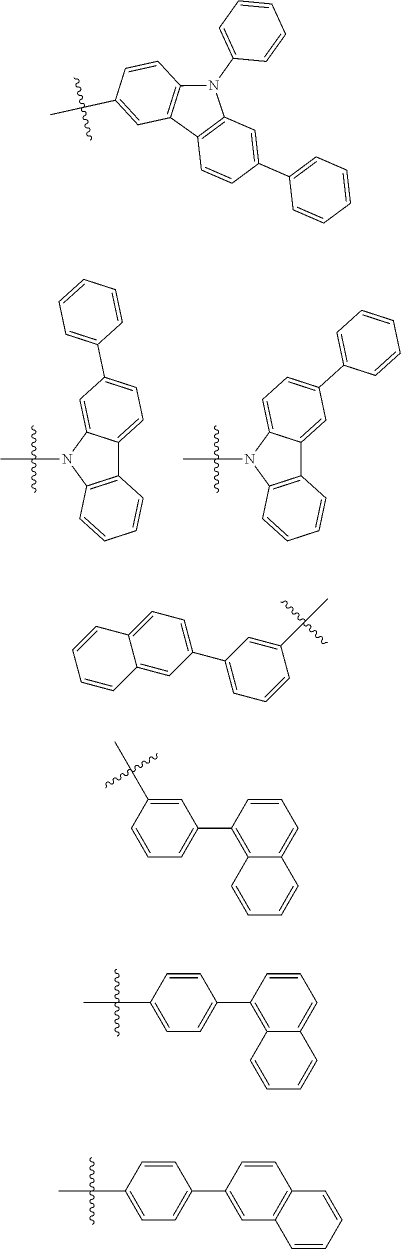 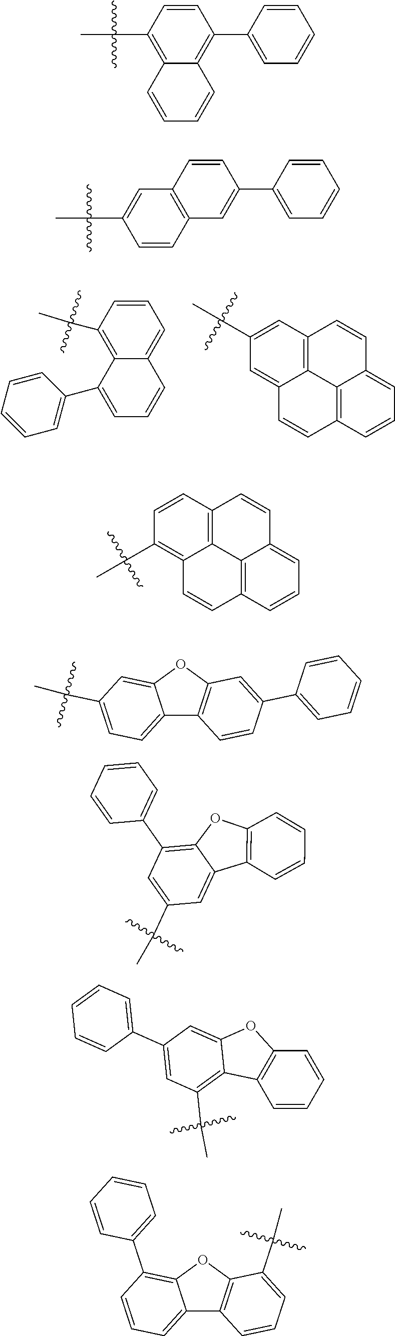 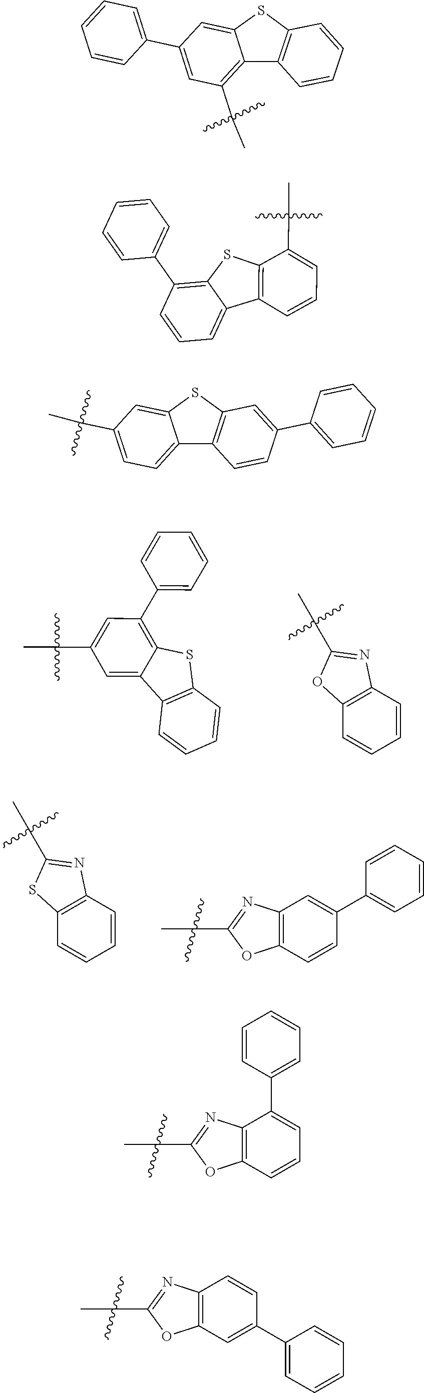 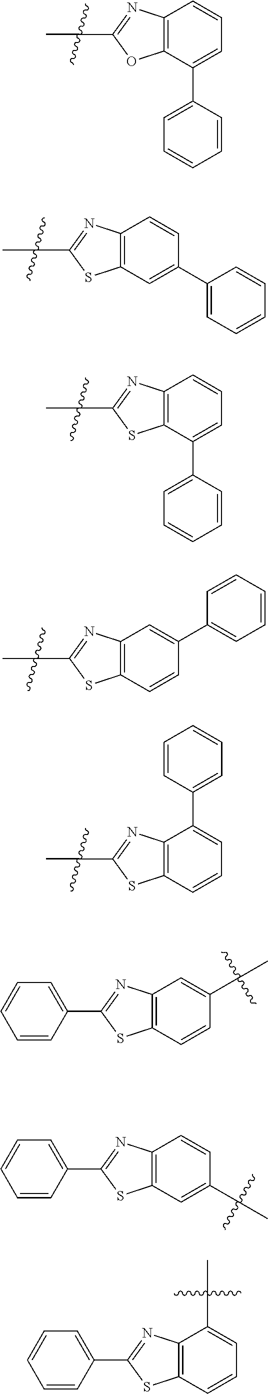 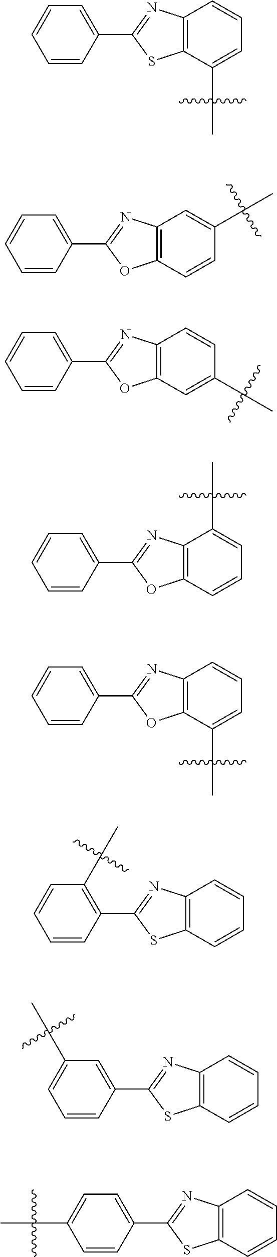 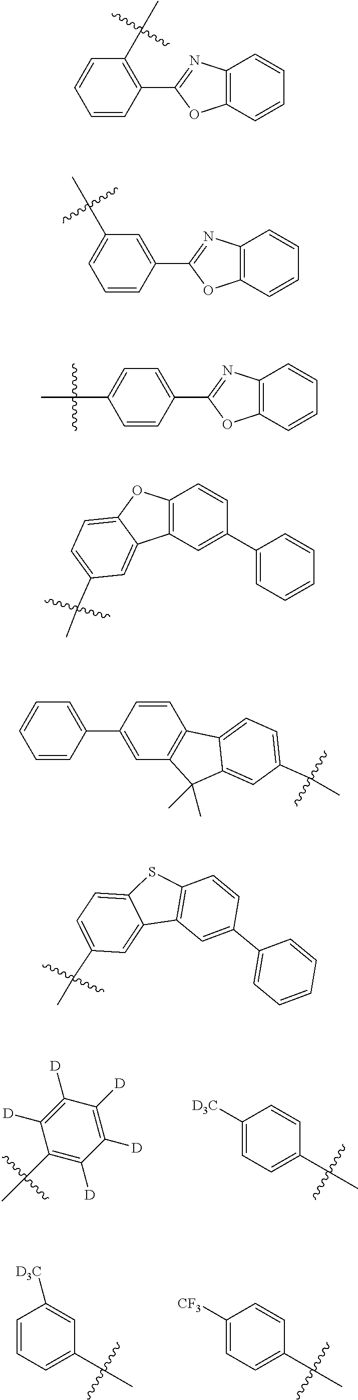 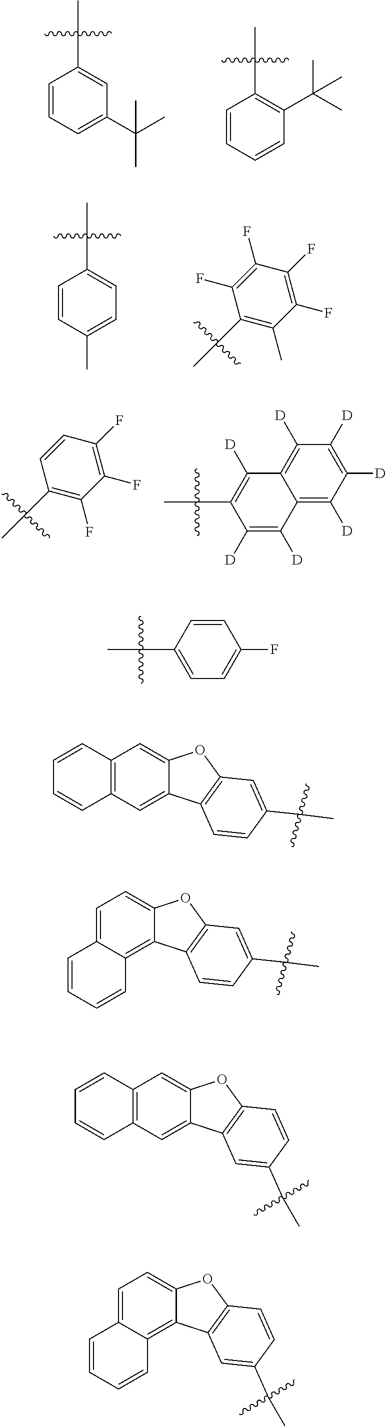 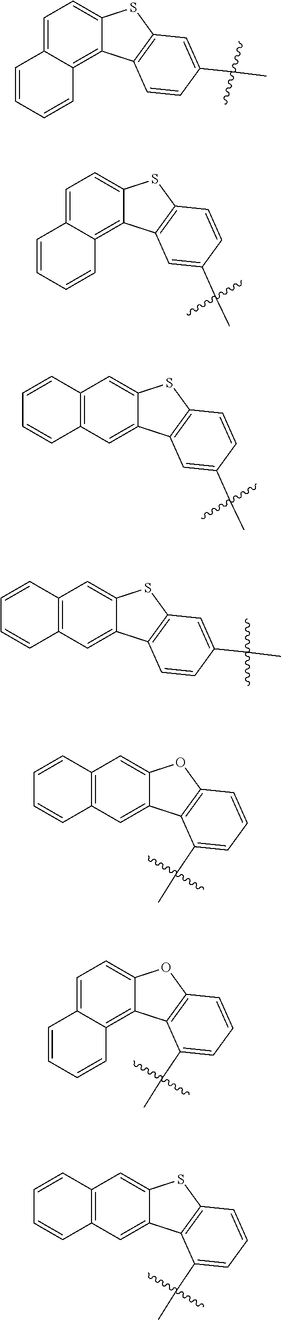 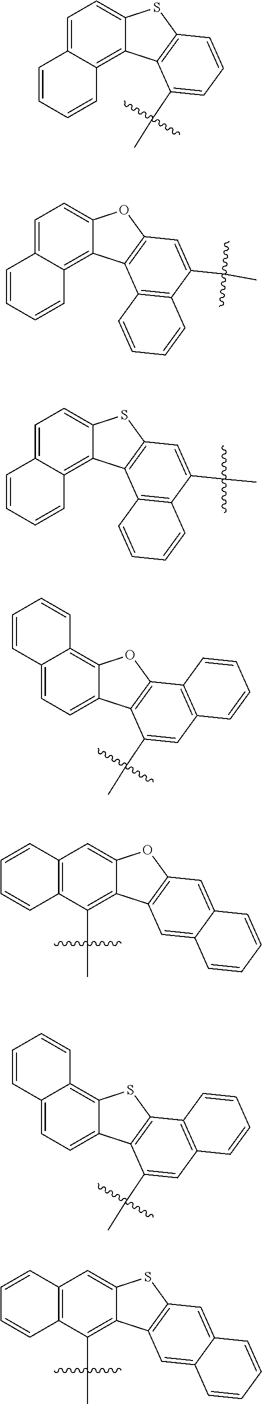 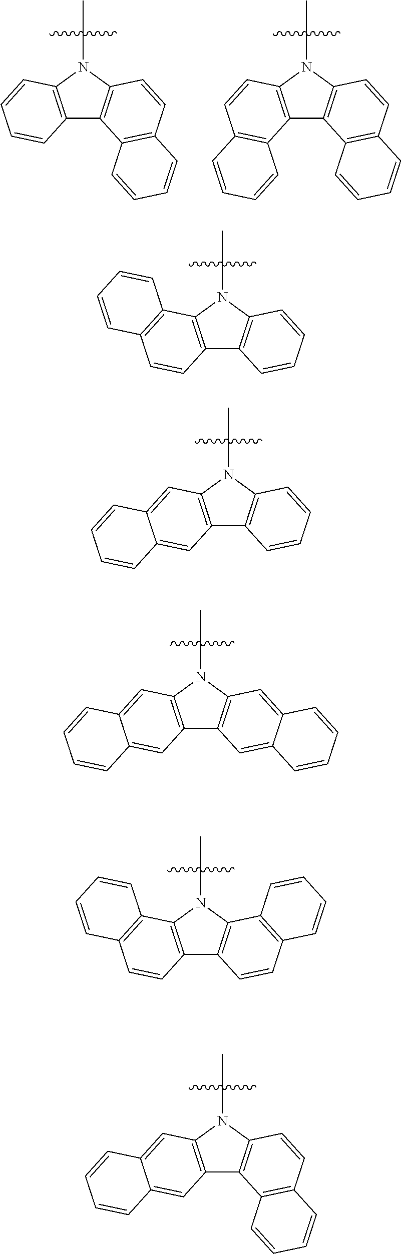 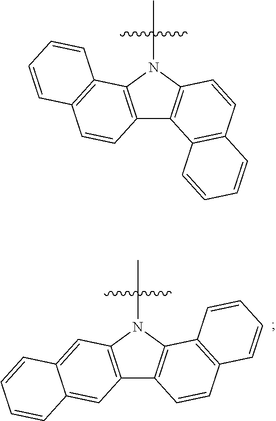 [SMILES: *n1c2ccc3ccccc3c2c2ccc3ccccc3c21 ; *n1c2cc3ccccc3cc2c2ccc3ccccc3c21]
Ar₃ is selected from the group consisting of the following groups:

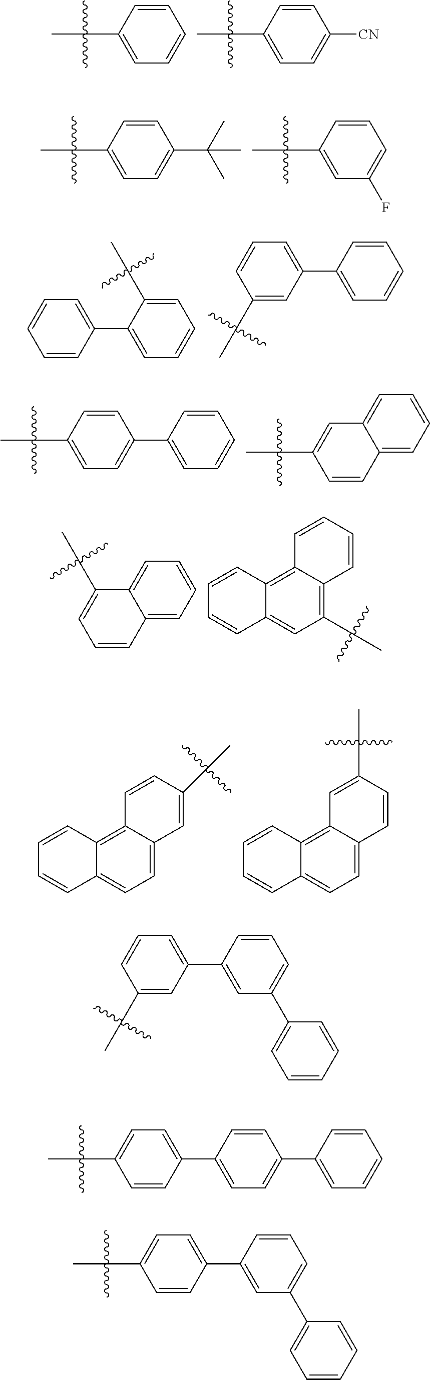

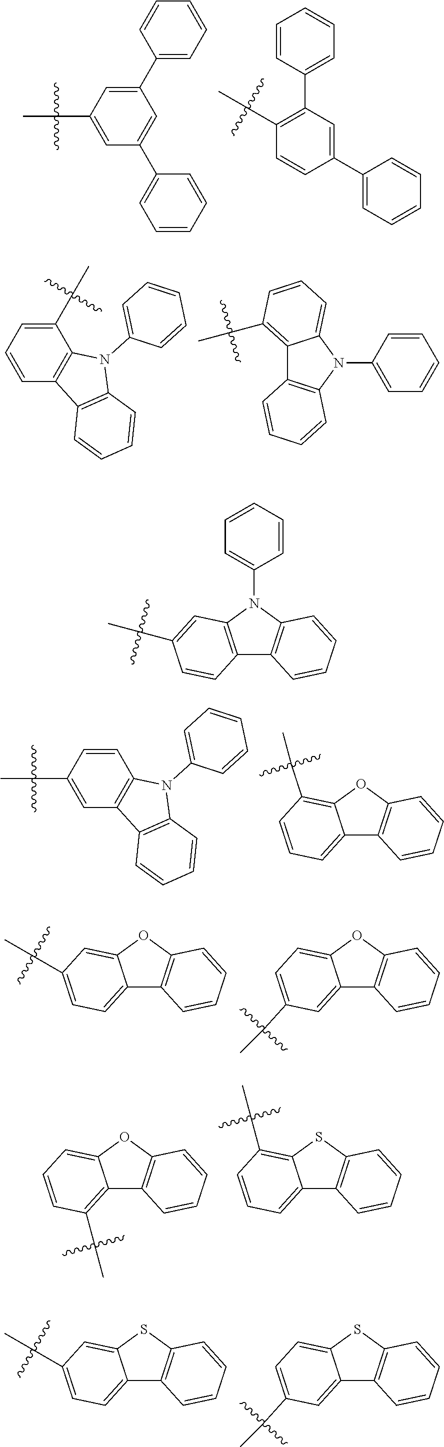

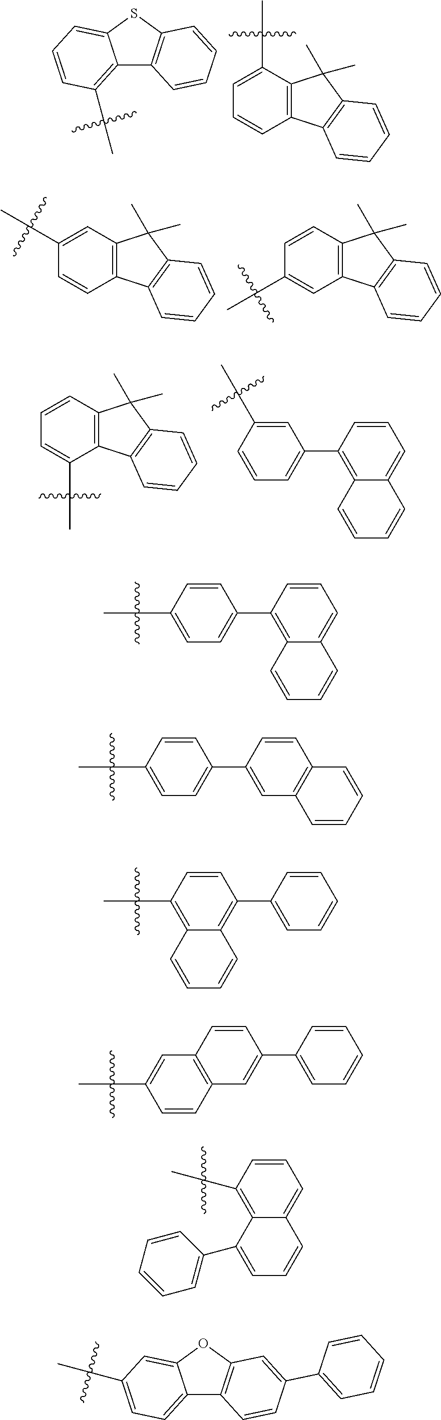

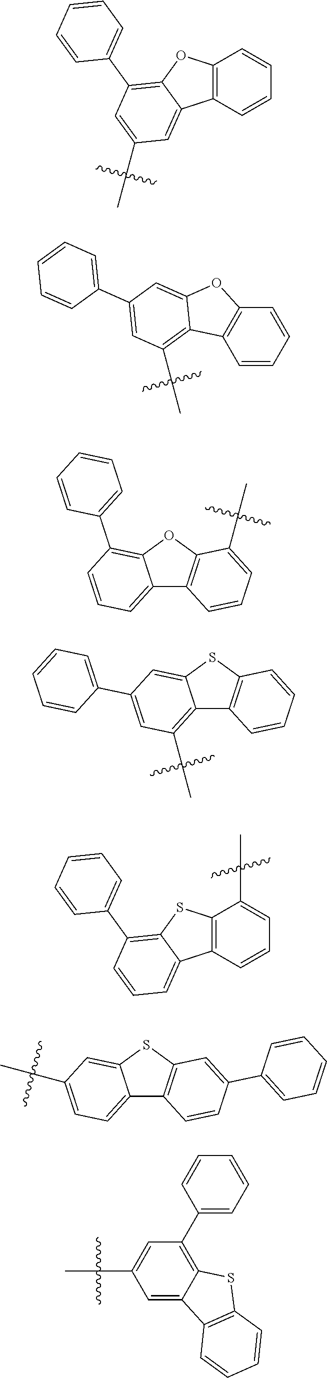

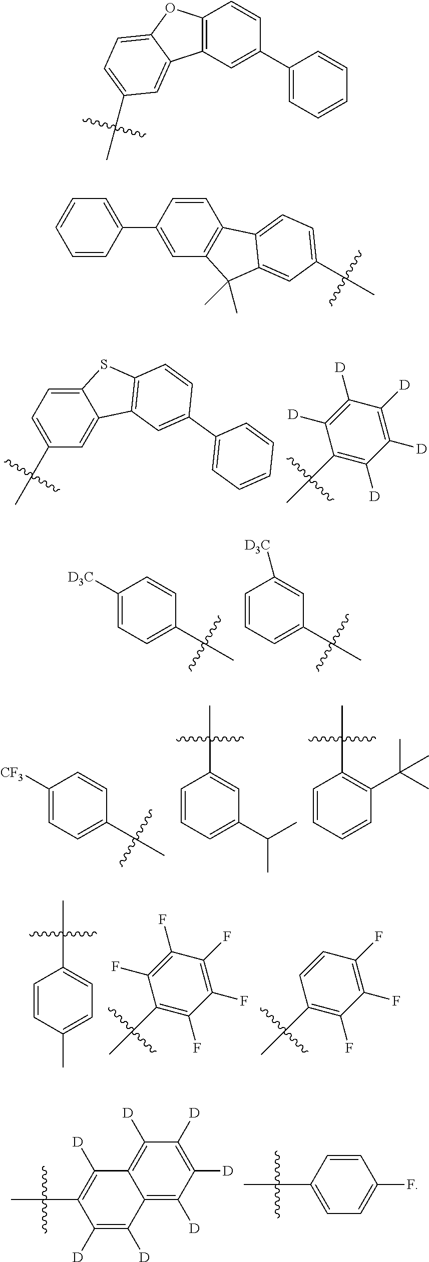

9. The nitrogen-containing compound according to claim 1, wherein L is selected from single bond or the group consisting of the following groups: 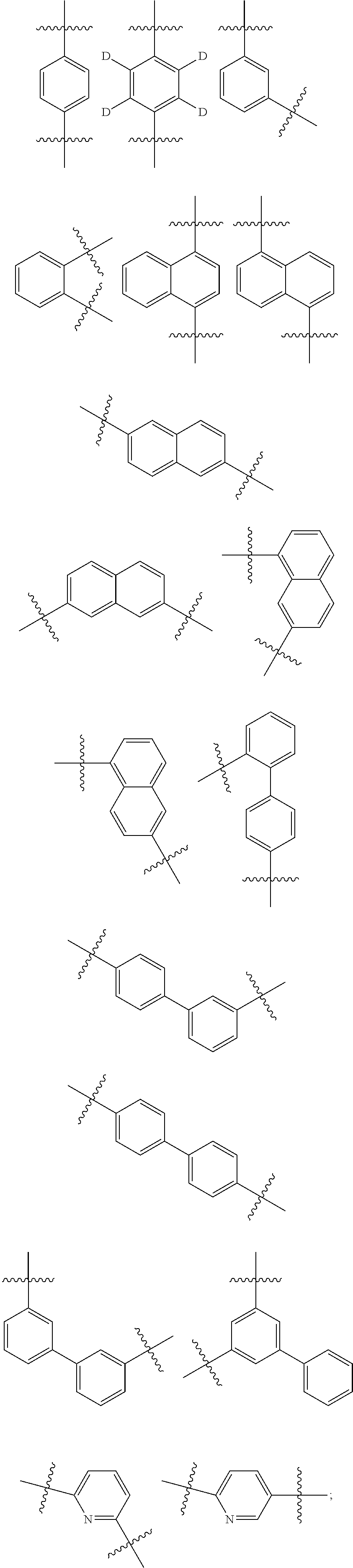
optionally, L₁ and L₂ are each independently selected from a single bond or the group consisting of the following groups:

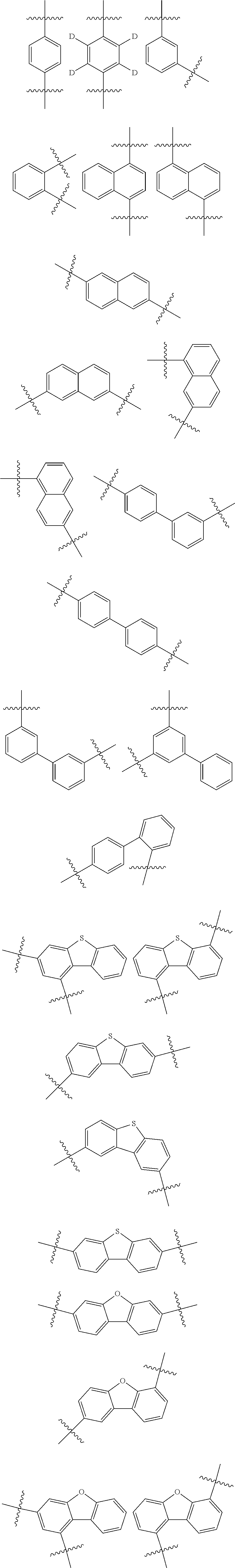

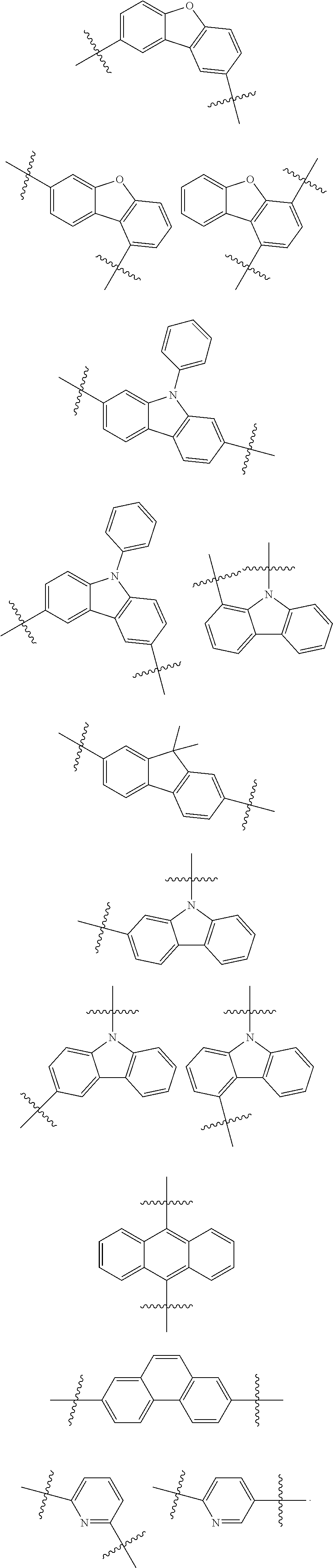

10. The nitrogen-containing compound according to claim 1, wherein 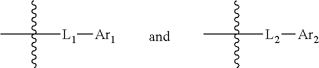
are each independently selected from the group consisting of the following groups: 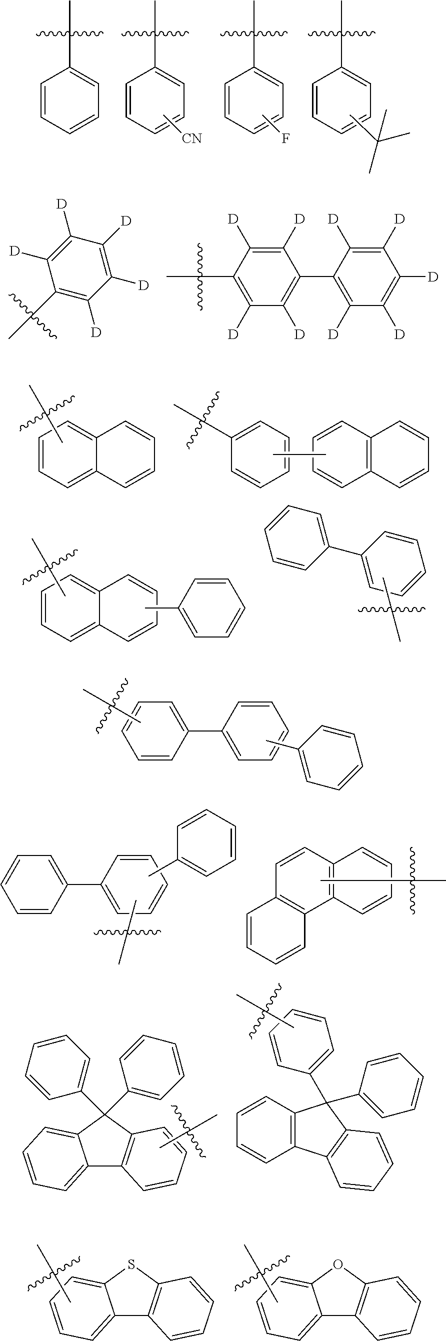 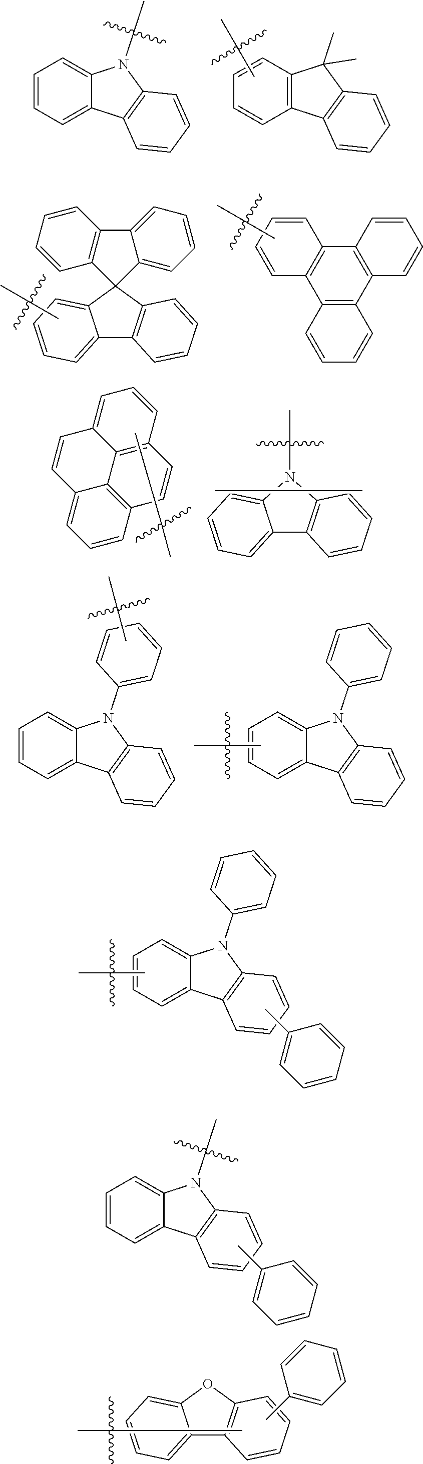 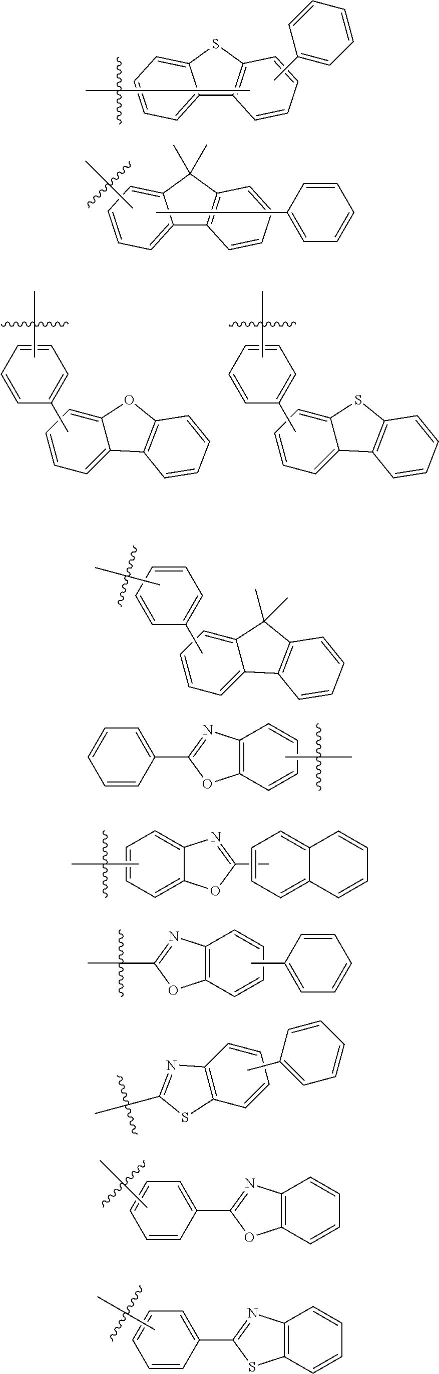 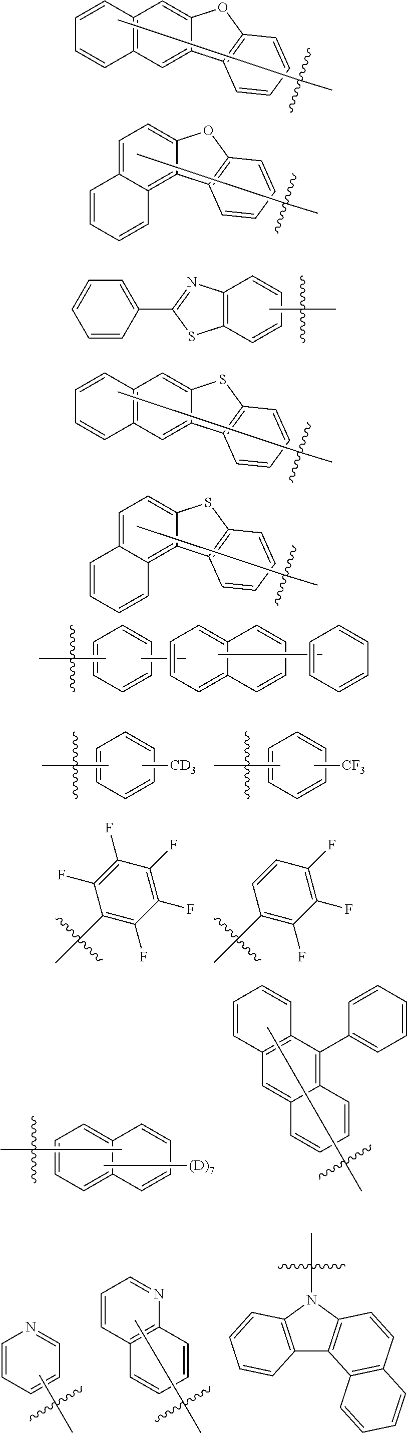 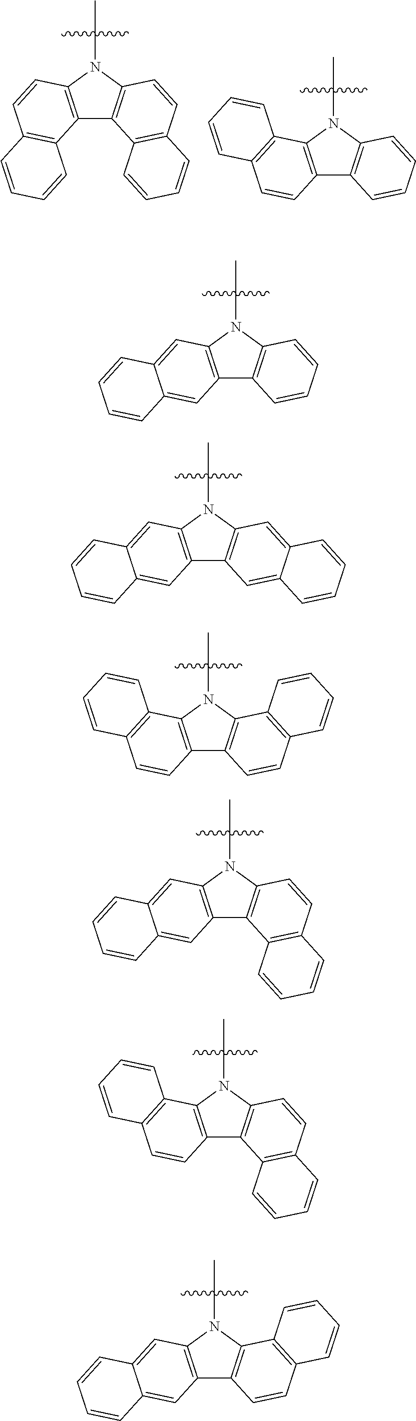 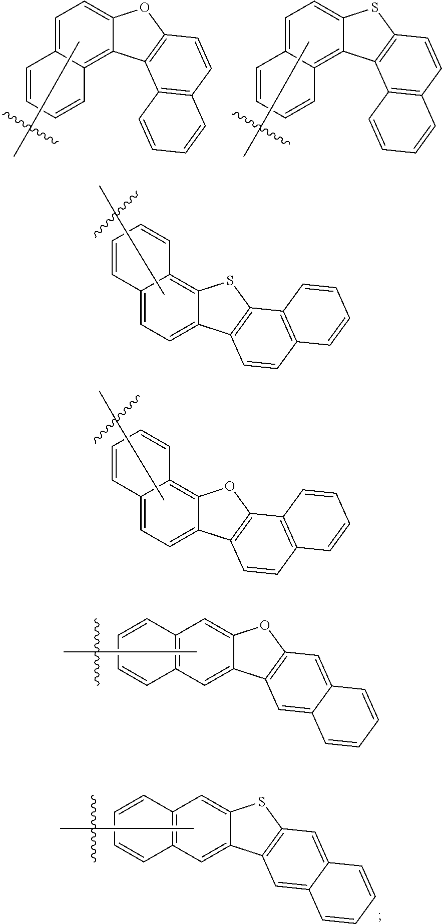
optionally, Ar₃ is selected from the group consisting of the following groups:

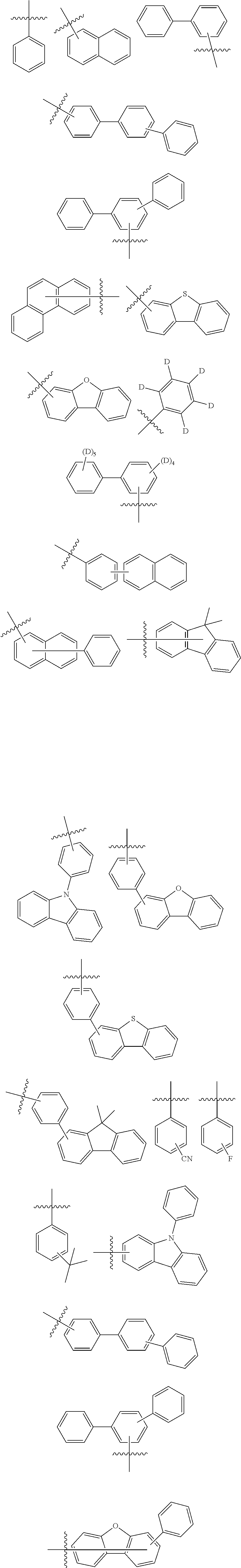

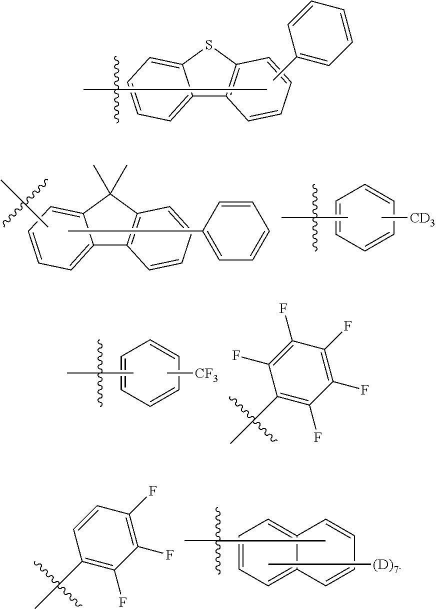

11. The nitrogen-containing compound according to claim 1, wherein each R is identical or different, and is each independently selected from hydrogen, deuterium, a cyano, a fluorine, a trideuteromethyl, a trimethylsilyl, a trifluoromethyl, a cyclopentyl, a cyclohexyl, an adamantyl, a methyl, an ethyl, an isopropyl, a tert-butyl, a phenyl, a naphthyl, a pyridyl, a dibenzofuranyl, a dibenzothiophenyl, or a carbazolyl.

12. The nitrogen-containing compound according to claim 1, wherein the nitrogen-containing compound is selected from the group consisting of the following compounds:

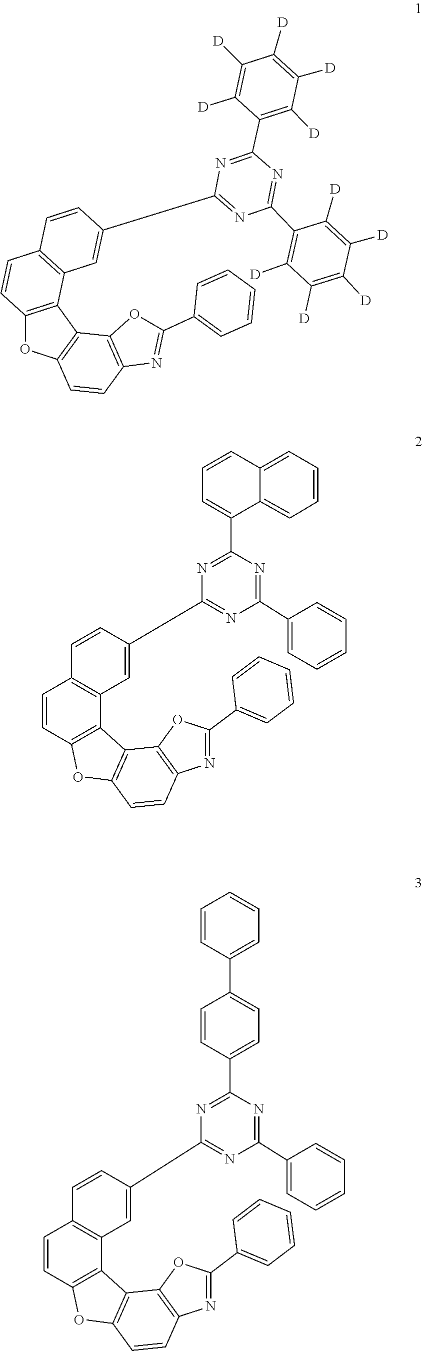 [SMILES: [2H]c1c([2H])c([2H])c(-c2nc(-c3ccc4ccc5oc6ccc7nc(-c8ccccc8)oc7c6c5c4c3)nc(-c3c([2H])c([2H])c([2H])c([2H])c3[2H])n2)c([2H])c1[2H] ; c1ccc(-c2nc(-c3ccc4ccc5oc6ccc7nc(-c8ccccc8)oc7c6c5c4c3)nc(-c3cccc4ccccc34)n2)cc1 ; c1ccc(-c2ccc(-c3nc(-c4ccccc4)nc(-c4ccc5ccc6oc7ccc8nc(-c9ccccc9)oc8c7c6c5c4)n3)cc2)cc1]

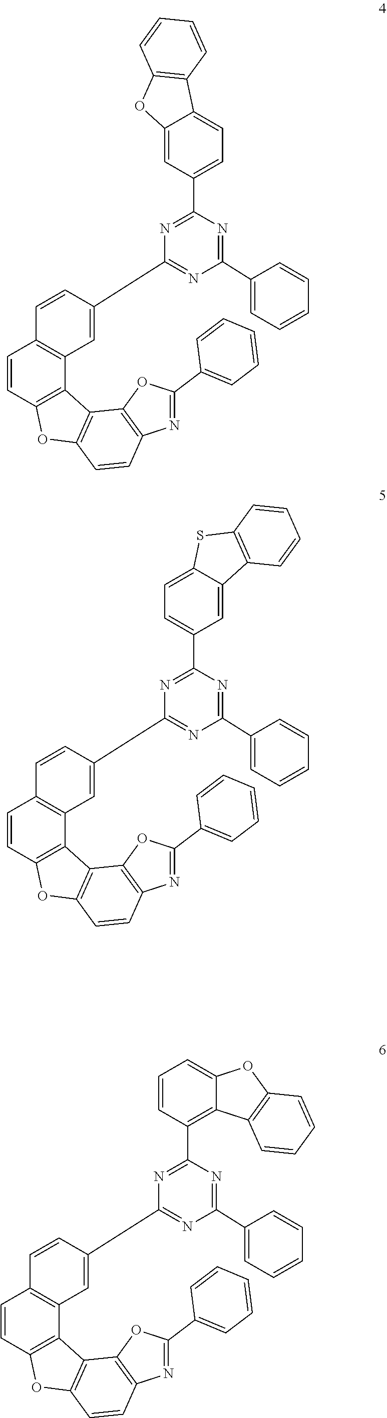 [SMILES: c1ccc(-c2nc(-c3ccc4c(c3)oc3ccccc34)nc(-c3ccc4ccc5oc6ccc7nc(-c8ccccc8)oc7c6c5c4c3)n2)cc1 ; c1ccc(-c2nc(-c3ccc4sc5ccccc5c4c3)nc(-c3ccc4ccc5oc6ccc7nc(-c8ccccc8)oc7c6c5c4c3)n2)cc1 ; c1ccc(-c2nc(-c3ccc4ccc5oc6ccc7nc(-c8ccccc8)oc7c6c5c4c3)nc(-c3cccc4oc5ccccc5c34)n2)cc1]

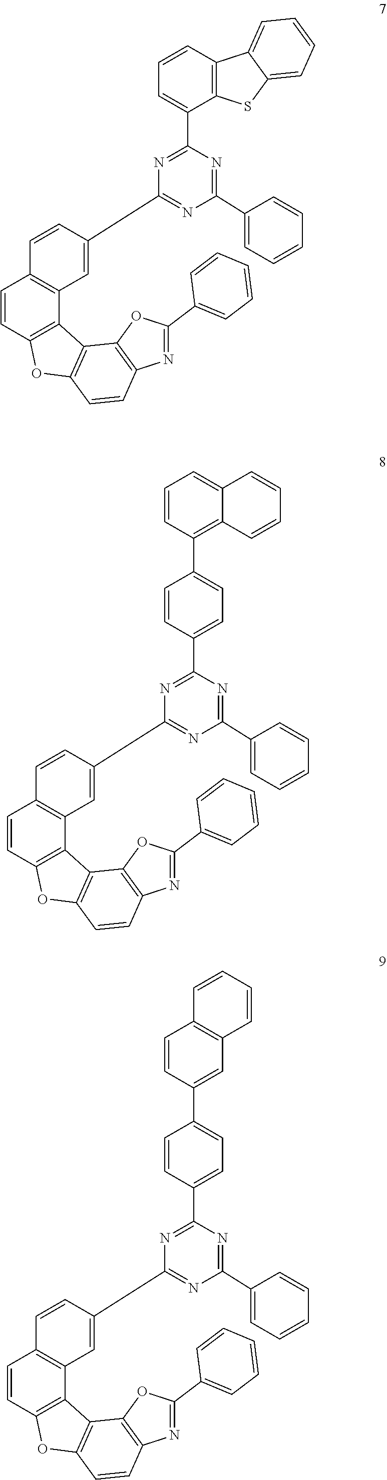 [SMILES: c1ccc(-c2nc(-c3ccc4ccc5oc6ccc7nc(-c8ccccc8)oc7c6c5c4c3)nc(-c3cccc4c3sc3ccccc34)n2)cc1 ; c1ccc(-c2nc(-c3ccc(-c4cccc5ccccc45)cc3)nc(-c3ccc4ccc5oc6ccc7nc(-c8ccccc8)oc7c6c5c4c3)n2)cc1 ; c1ccc(-c2nc(-c3ccc(-c4ccc5ccccc5c4)cc3)nc(-c3ccc4ccc5oc6ccc7nc(-c8ccccc8)oc7c6c5c4c3)n2)cc1]

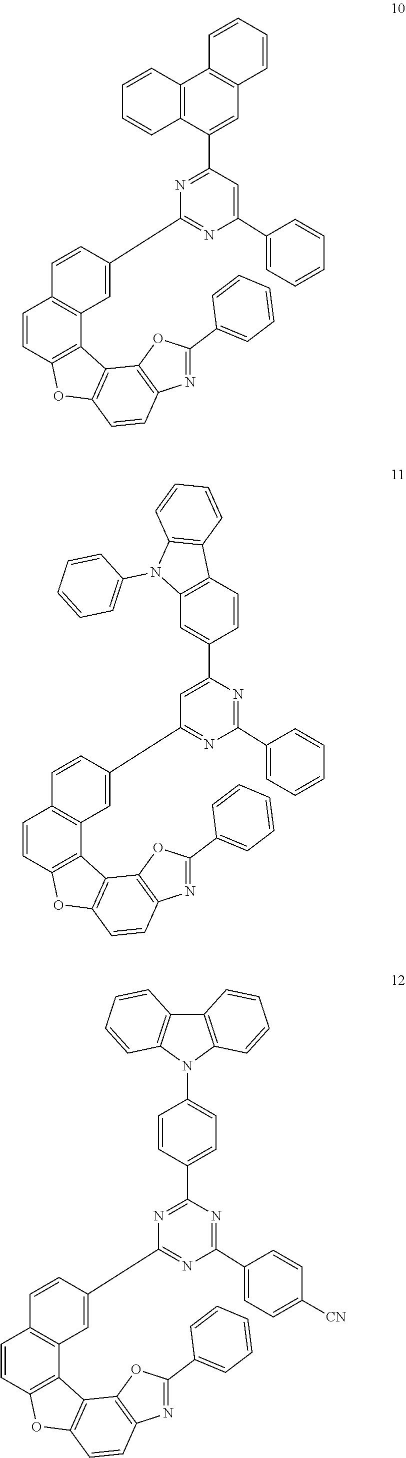 [SMILES: c1ccc(-c2cc(-c3cc4ccccc4c4ccccc34)nc(-c3ccc4ccc5oc6ccc7nc(-c8ccccc8)oc7c6c5c4c3)n2)cc1 ; c1ccc(-c2nc(-c3ccc4ccc5oc6ccc7nc(-c8ccccc8)oc7c6c5c4c3)cc(-c3ccc4c5ccccc5n(-c5ccccc5)c4c3)n2)cc1 ; N#Cc1ccc(-c2nc(-c3ccc(-n4c5ccccc5c5ccccc54)cc3)nc(-c3ccc4ccc5oc6ccc7nc(-c8ccccc8)oc7c6c5c4c3)n2)cc1]

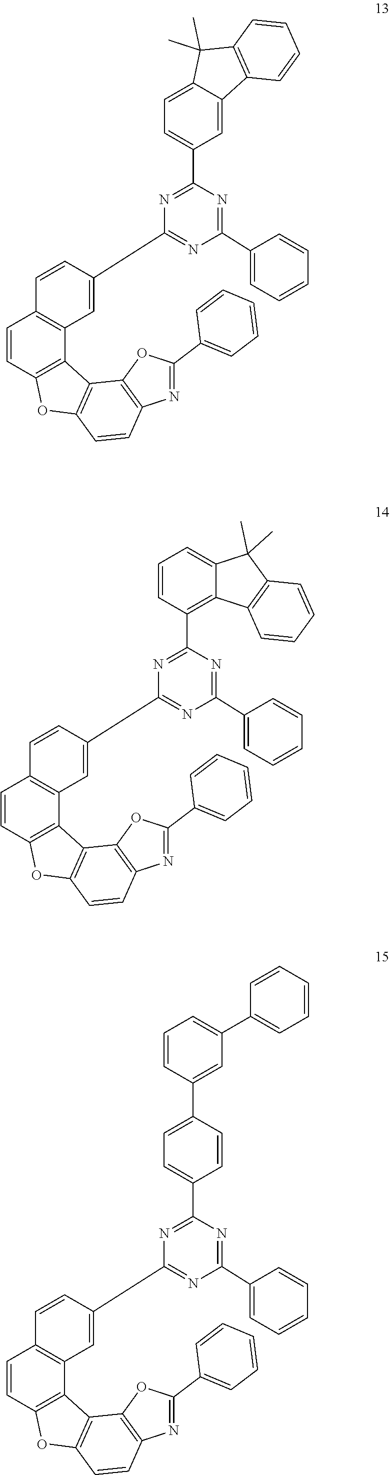 [SMILES: CC1(C)c2ccccc2-c2cc(-c3nc(-c4ccccc4)nc(-c4ccc5ccc6oc7ccc8nc(-c9ccccc9)oc8c7c6c5c4)n3)ccc21 ; CC1(C)c2ccccc2-c2c(-c3nc(-c4ccccc4)nc(-c4ccc5ccc6oc7ccc8nc(-c9ccccc9)oc8c7c6c5c4)n3)cccc21 ; c1ccc(-c2cccc(-c3ccc(-c4nc(-c5ccccc5)nc(-c5ccc6ccc7oc8ccc9nc(-c%10ccccc%10)oc9c8c7c6c5)n4)cc3)c2)cc1]

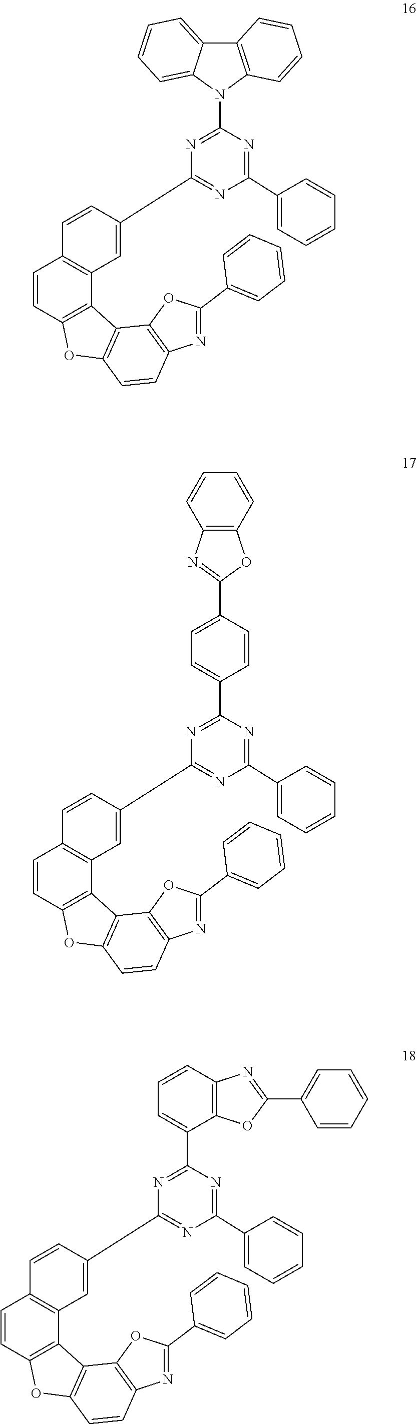 [SMILES: c1ccc(-c2nc(-c3ccc4ccc5oc6ccc7nc(-c8ccccc8)oc7c6c5c4c3)nc(-n3c4ccccc4c4ccccc43)n2)cc1 ; c1ccc(-c2nc(-c3ccc(-c4nc5ccccc5o4)cc3)nc(-c3ccc4ccc5oc6ccc7nc(-c8ccccc8)oc7c6c5c4c3)n2)cc1 ; c1ccc(-c2nc(-c3ccc4ccc5oc6ccc7nc(-c8ccccc8)oc7c6c5c4c3)nc(-c3cccc4nc(-c5ccccc5)oc34)n2)cc1]

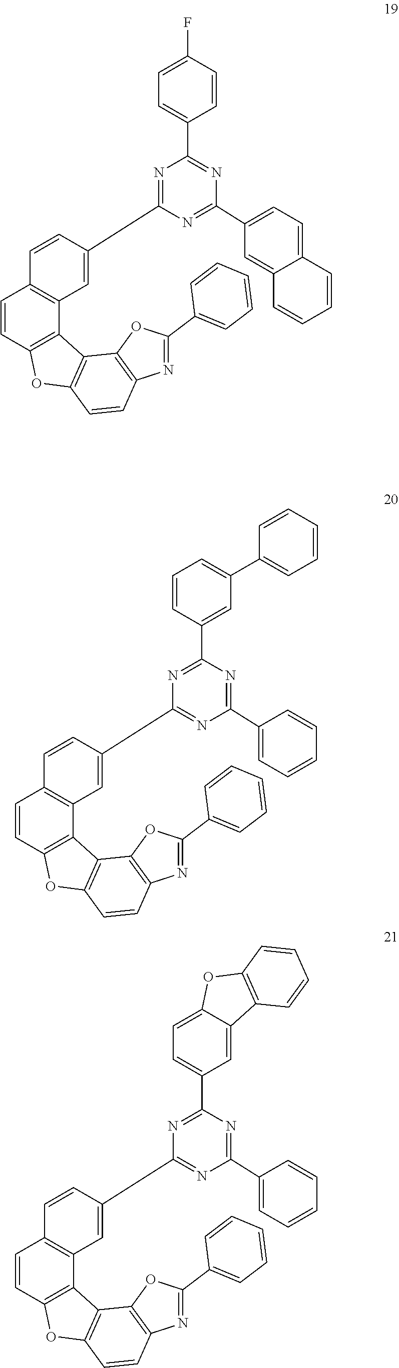 [SMILES: Fc1ccc(-c2nc(-c3ccc4ccccc4c3)nc(-c3ccc4ccc5oc6ccc7nc(-c8ccccc8)oc7c6c5c4c3)n2)cc1 ; c1ccc(-c2cccc(-c3nc(-c4ccccc4)nc(-c4ccc5ccc6oc7ccc8nc(-c9ccccc9)oc8c7c6c5c4)n3)c2)cc1 ; c1ccc(-c2nc(-c3ccc4oc5ccccc5c4c3)nc(-c3ccc4ccc5oc6ccc7nc(-c8ccccc8)oc7c6c5c4c3)n2)cc1]

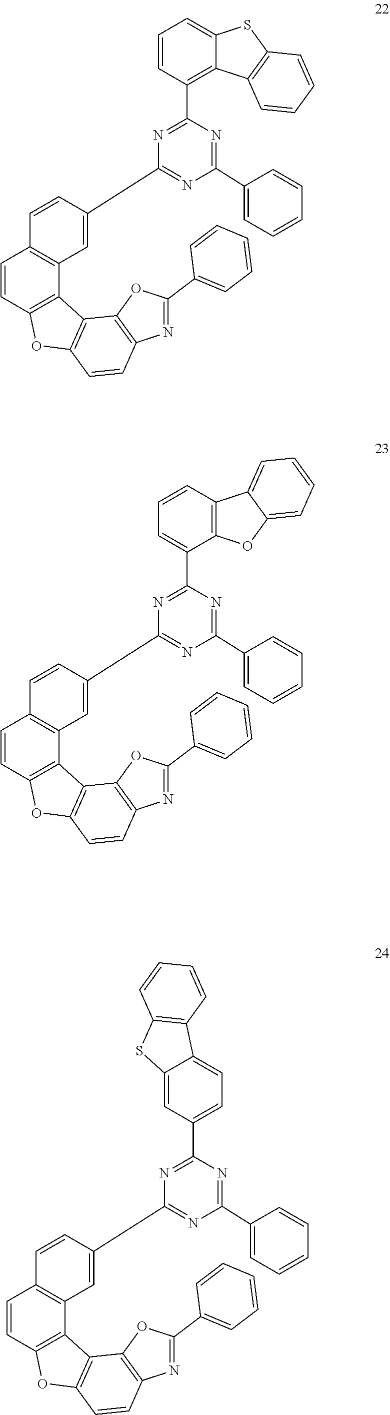 [SMILES: c1ccc(-c2nc(-c3ccc4ccc5oc6ccc7nc(-c8ccccc8)oc7c6c5c4c3)nc(-c3cccc4sc5ccccc5c34)n2)cc1 ; c1ccc(-c2nc(-c3ccc4ccc5oc6ccc7nc(-c8ccccc8)oc7c6c5c4c3)nc(-c3cccc4c3oc3ccccc34)n2)cc1 ; c1ccc(-c2nc(-c3ccc4c(c3)sc3ccccc34)nc(-c3ccc4ccc5oc6ccc7nc(-c8ccccc8)oc7c6c5c4c3)n2)cc1]

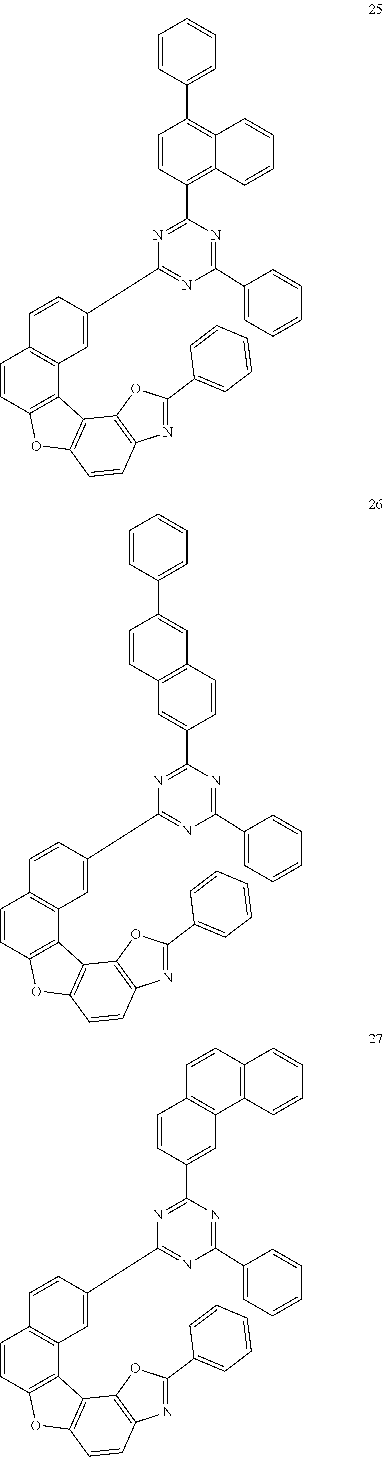 [SMILES: c1ccc(-c2nc(-c3ccc4ccc5oc6ccc7nc(-c8ccccc8)oc7c6c5c4c3)nc(-c3ccc(-c4ccccc4)c4ccccc34)n2)cc1 ; c1ccc(-c2ccc3cc(-c4nc(-c5ccccc5)nc(-c5ccc6ccc7oc8ccc9nc(-c%10ccccc%10)oc9c8c7c6c5)n4)ccc3c2)cc1 ; c1ccc(-c2nc(-c3ccc4ccc5ccccc5c4c3)nc(-c3ccc4ccc5oc6ccc7nc(-c8ccccc8)oc7c6c5c4c3)n2)cc1]

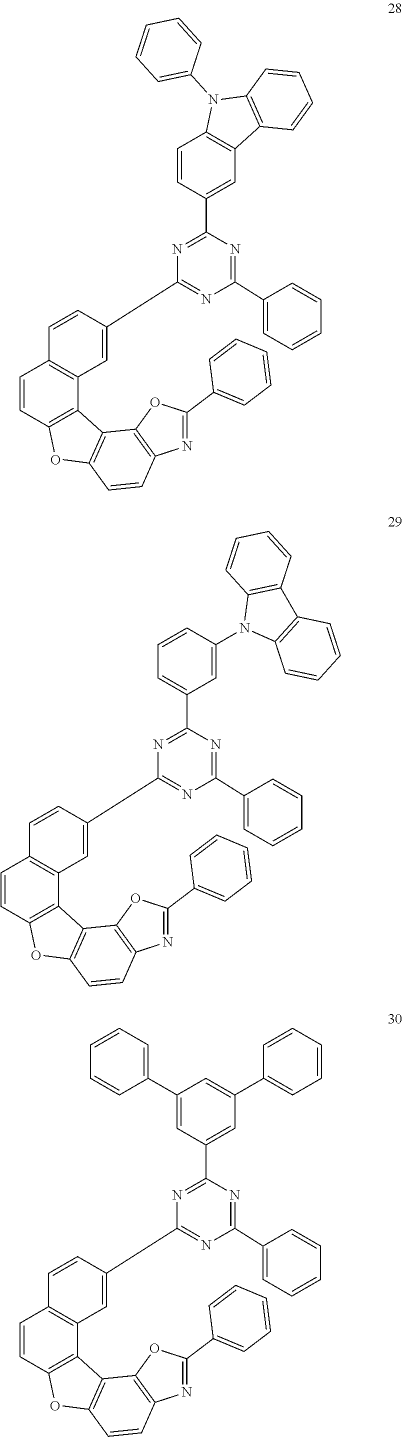

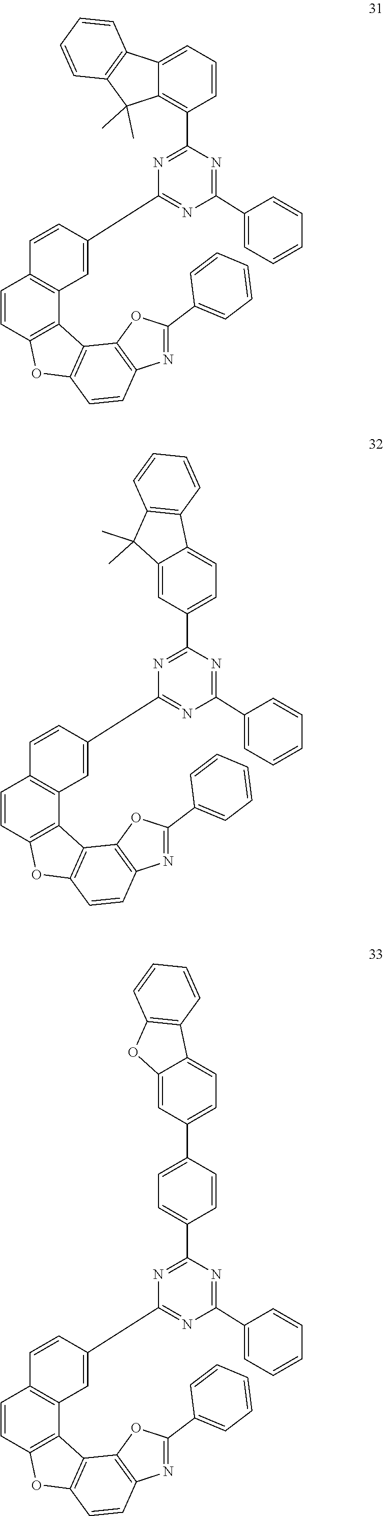 [SMILES: CC1(C)c2ccccc2-c2cccc(-c3nc(-c4ccccc4)nc(-c4ccc5ccc6oc7ccc8nc(-c9ccccc9)oc8c7c6c5c4)n3)c21 ; CC1(C)c2ccccc2-c2ccc(-c3nc(-c4ccccc4)nc(-c4ccc5ccc6oc7ccc8nc(-c9ccccc9)oc8c7c6c5c4)n3)cc21 ; c1ccc(-c2nc(-c3ccc(-c4ccc5c(c4)oc4ccccc45)cc3)nc(-c3ccc4ccc5oc6ccc7nc(-c8ccccc8)oc7c6c5c4c3)n2)cc1]

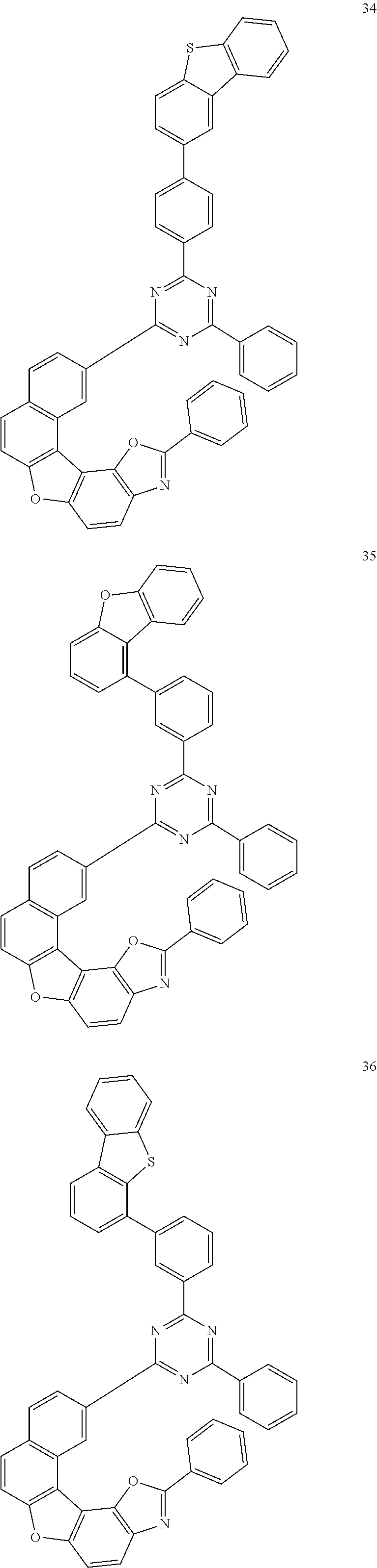 [SMILES: c1ccc(-c2nc(-c3ccc(-c4ccc5sc6ccccc6c5c4)cc3)nc(-c3ccc4ccc5oc6ccc7nc(-c8ccccc8)oc7c6c5c4c3)n2)cc1 ; c1ccc(-c2nc(-c3cccc(-c4cccc5oc6ccccc6c45)c3)nc(-c3ccc4ccc5oc6ccc7nc(-c8ccccc8)oc7c6c5c4c3)n2)cc1 ; c1ccc(-c2nc(-c3cccc(-c4cccc5c4sc4ccccc45)c3)nc(-c3ccc4ccc5oc6ccc7nc(-c8ccccc8)oc7c6c5c4c3)n2)cc1]

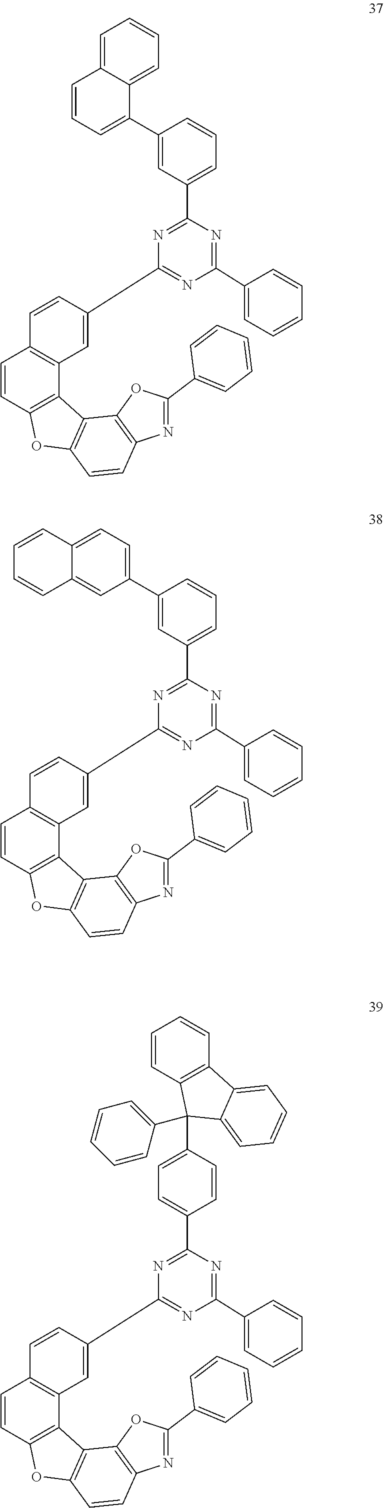 [SMILES: c1ccc(-c2nc(-c3cccc(-c4cccc5ccccc45)c3)nc(-c3ccc4ccc5oc6ccc7nc(-c8ccccc8)oc7c6c5c4c3)n2)cc1 ; c1ccc(-c2nc(-c3cccc(-c4ccc5ccccc5c4)c3)nc(-c3ccc4ccc5oc6ccc7nc(-c8ccccc8)oc7c6c5c4c3)n2)cc1 ; c1ccc(-c2nc(-c3ccc(C4(c5ccccc5)c5ccccc5-c5ccccc54)cc3)nc(-c3ccc4ccc5oc6ccc7nc(-c8ccccc8)oc7c6c5c4c3)n2)cc1]

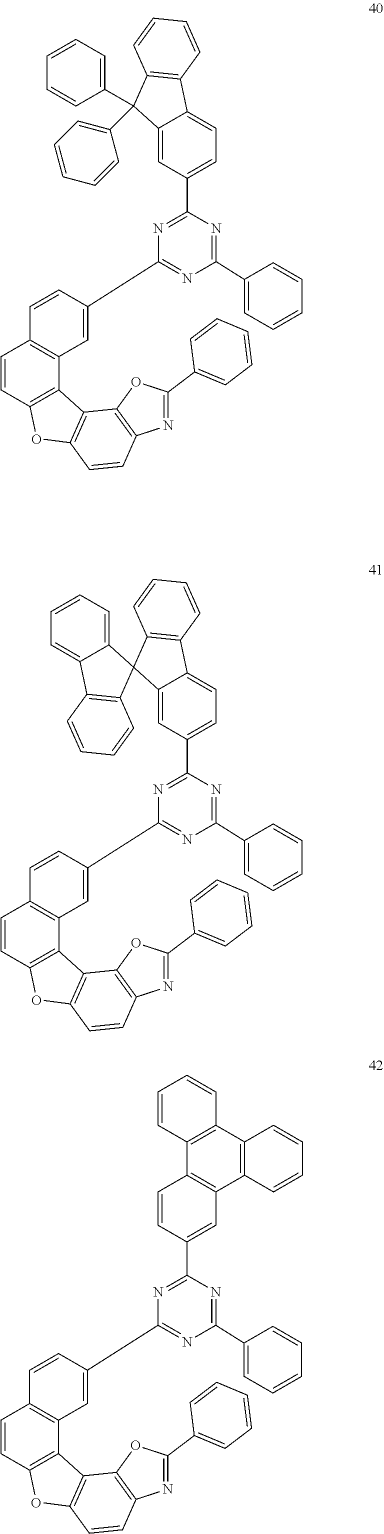 [SMILES: c1ccc(-c2nc(-c3ccc4c(c3)C(c3ccccc3)(c3ccccc3)c3ccccc3-4)nc(-c3ccc4ccc5oc6ccc7nc(-c8ccccc8)oc7c6c5c4c3)n2)cc1 ; c1ccc(-c2nc(-c3ccc4c(c3)C3(c5ccccc5-c5ccccc53)c3ccccc3-4)nc(-c3ccc4ccc5oc6ccc7nc(-c8ccccc8)oc7c6c5c4c3)n2)cc1 ; c1ccc(-c2nc(-c3ccc4c5ccccc5c5ccccc5c4c3)nc(-c3ccc4ccc5oc6ccc7nc(-c8ccccc8)oc7c6c5c4c3)n2)cc1]

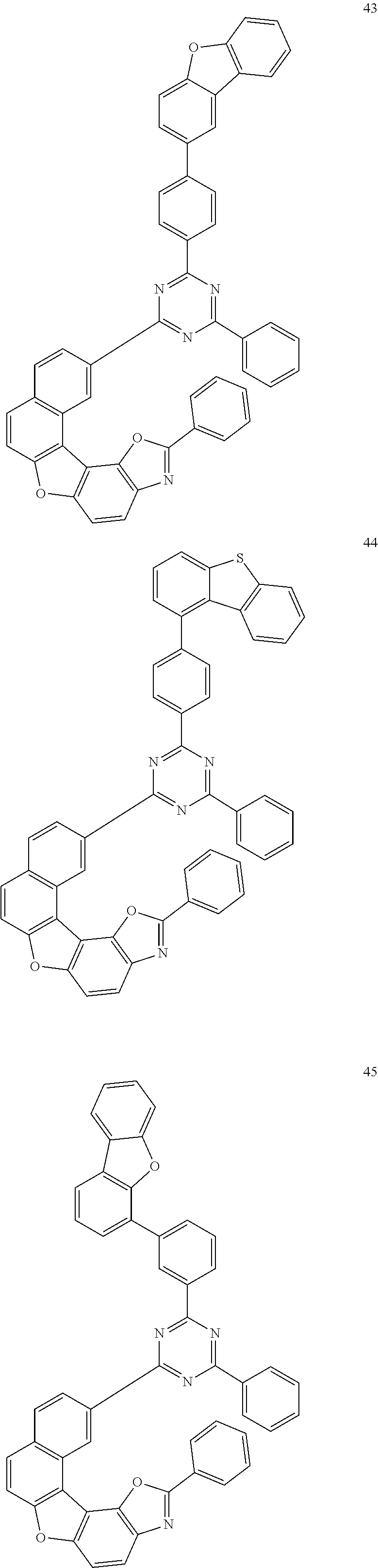 [SMILES: c1ccc(-c2nc(-c3ccc(-c4ccc5oc6ccccc6c5c4)cc3)nc(-c3ccc4ccc5oc6ccc7nc(-c8ccccc8)oc7c6c5c4c3)n2)cc1 ; c1ccc(-c2nc(-c3ccc(-c4cccc5sc6ccccc6c45)cc3)nc(-c3ccc4ccc5oc6ccc7nc(-c8ccccc8)oc7c6c5c4c3)n2)cc1 ; c1ccc(-c2nc(-c3cccc(-c4cccc5c4oc4ccccc45)c3)nc(-c3ccc4ccc5oc6ccc7nc(-c8ccccc8)oc7c6c5c4c3)n2)cc1]

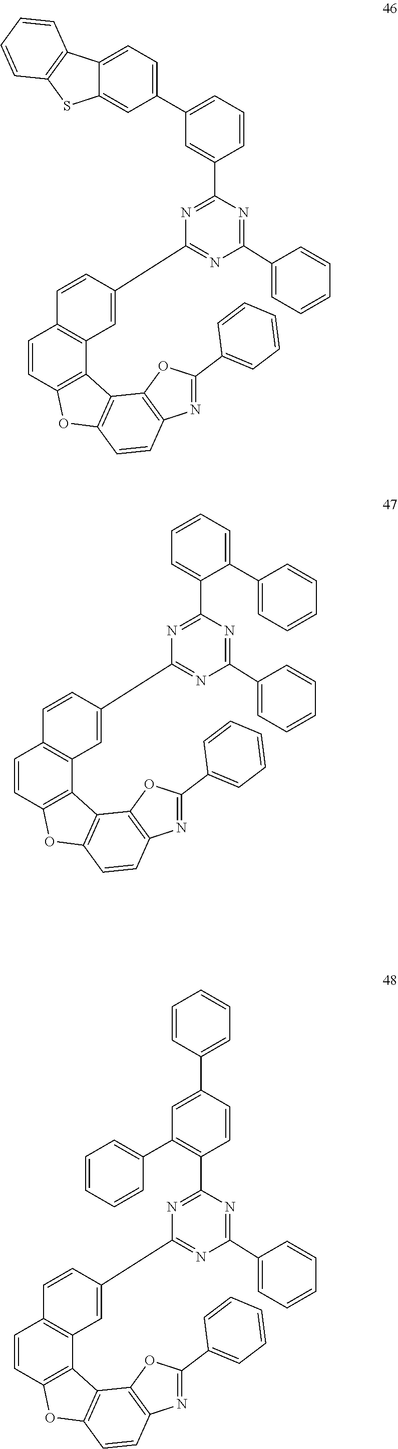 [SMILES: c1ccc(-c2nc(-c3cccc(-c4ccc5c(c4)sc4ccccc45)c3)nc(-c3ccc4ccc5oc6ccc7nc(-c8ccccc8)oc7c6c5c4c3)n2)cc1 ; c1ccc(-c2nc(-c3ccc4ccc5oc6ccc7nc(-c8ccccc8)oc7c6c5c4c3)nc(-c3ccccc3-c3ccccc3)n2)cc1 ; c1ccc(-c2ccc(-c3nc(-c4ccccc4)nc(-c4ccc5ccc6oc7ccc8nc(-c9ccccc9)oc8c7c6c5c4)n3)c(-c3ccccc3)c2)cc1]

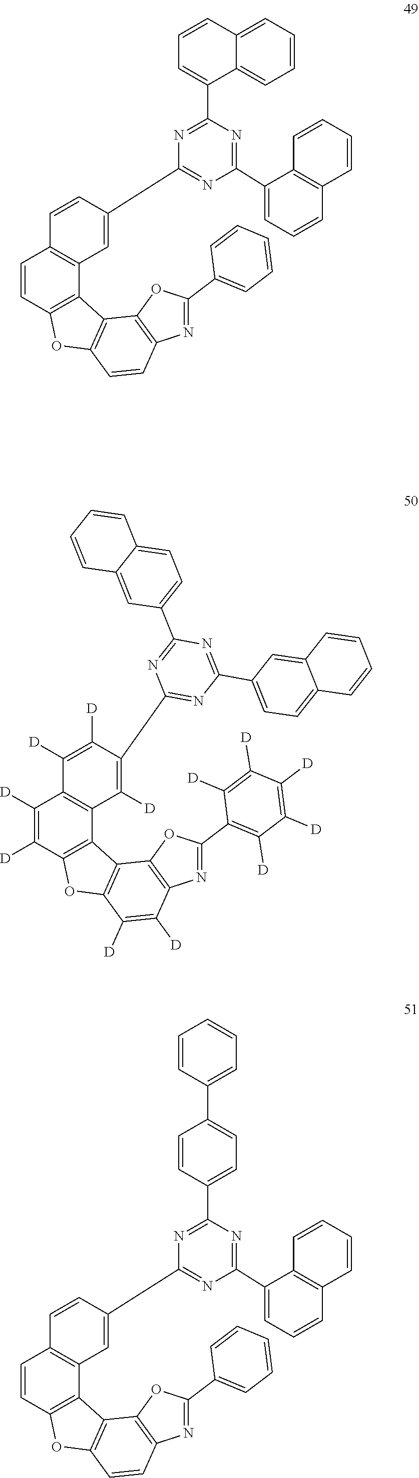 [SMILES: c1ccc(-c2nc3ccc4oc5ccc6ccc(-c7nc(-c8cccc9ccccc89)nc(-c8cccc9ccccc89)n7)cc6c5c4c3o2)cc1 ; [2H]c1c([2H])c([2H])c(-c2nc3c([2H])c([2H])c4oc5c([2H])c([2H])c6c([2H])c([2H])c(-c7nc(-c8ccc9ccccc9c8)nc(-c8ccc9ccccc9c8)n7)c([2H])c6c5c4c3o2)c([2H])c1[2H] ; c1ccc(-c2ccc(-c3nc(-c4ccc5ccc6oc7ccc8nc(-c9ccccc9)oc8c7c6c5c4)nc(-c4cccc5ccccc45)n3)cc2)cc1]

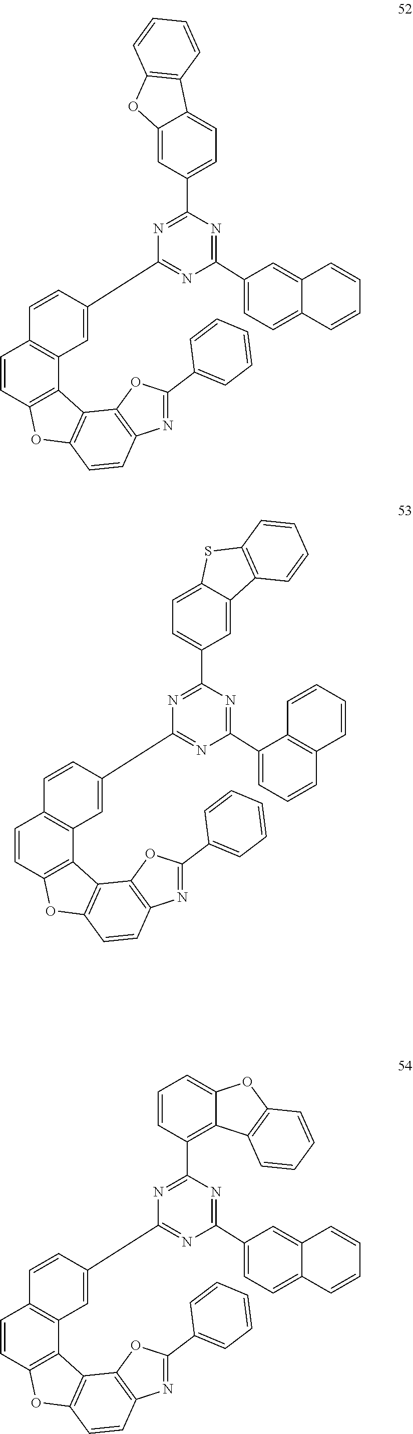 [SMILES: c1ccc(-c2nc3ccc4oc5ccc6ccc(-c7nc(-c8ccc9ccccc9c8)nc(-c8ccc9c(c8)oc8ccccc89)n7)cc6c5c4c3o2)cc1 ; c1ccc(-c2nc3ccc4oc5ccc6ccc(-c7nc(-c8ccc9sc%10ccccc%10c9c8)nc(-c8cccc9ccccc89)n7)cc6c5c4c3o2)cc1 ; c1ccc(-c2nc3ccc4oc5ccc6ccc(-c7nc(-c8ccc9ccccc9c8)nc(-c8cccc9oc%10ccccc%10c89)n7)cc6c5c4c3o2)cc1]

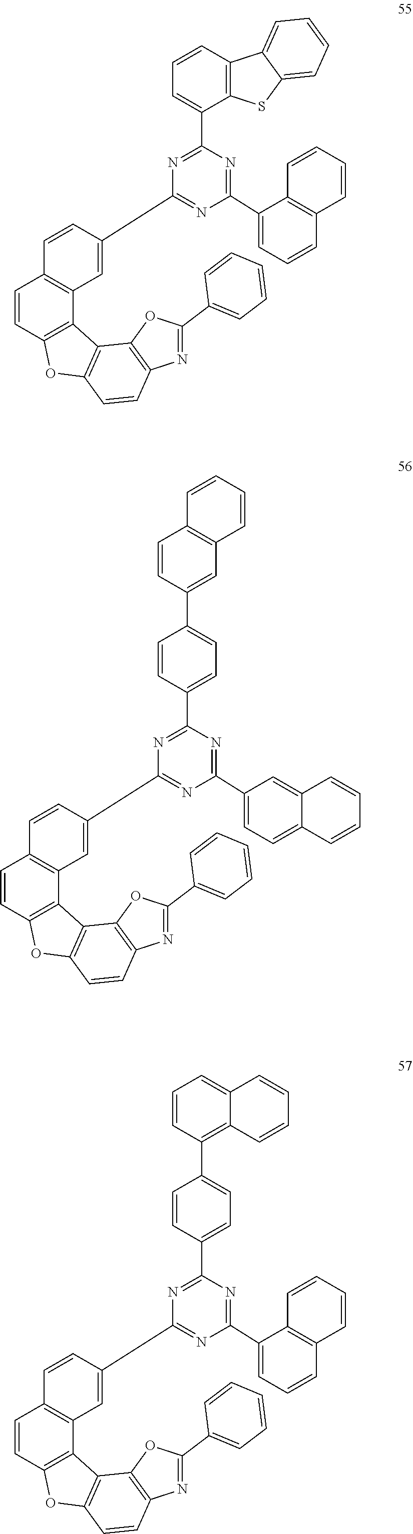 [SMILES: c1ccc(-c2nc3ccc4oc5ccc6ccc(-c7nc(-c8cccc9ccccc89)nc(-c8cccc9c8sc8ccccc89)n7)cc6c5c4c3o2)cc1 ; c1ccc(-c2nc3ccc4oc5ccc6ccc(-c7nc(-c8ccc(-c9ccc%10ccccc%10c9)cc8)nc(-c8ccc9ccccc9c8)n7)cc6c5c4c3o2)cc1 ; c1ccc(-c2nc3ccc4oc5ccc6ccc(-c7nc(-c8ccc(-c9cccc%10ccccc9%10)cc8)nc(-c8cccc9ccccc89)n7)cc6c5c4c3o2)cc1]

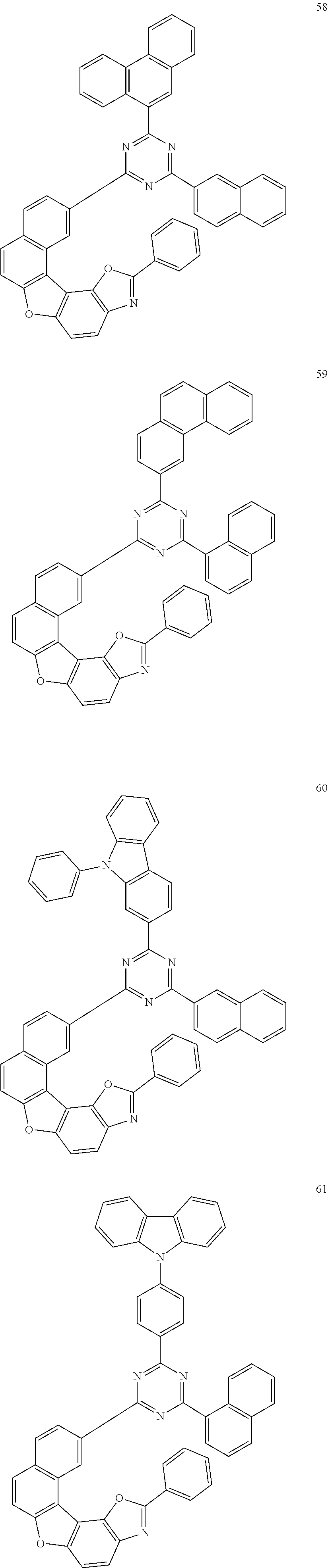 [SMILES: c1ccc(-c2nc3ccc4oc5ccc6ccc(-c7nc(-c8ccc9ccccc9c8)nc(-c8cc9ccccc9c9ccccc89)n7)cc6c5c4c3o2)cc1 ; c1ccc(-c2nc3ccc4oc5ccc6ccc(-c7nc(-c8ccc9ccc%10ccccc%10c9c8)nc(-c8cccc9ccccc89)n7)cc6c5c4c3o2)cc1 ; c1ccc(-c2nc3ccc4oc5ccc6ccc(-c7nc(-c8ccc9ccccc9c8)nc(-c8ccc9c%10ccccc%10n(-c%10ccccc%10)c9c8)n7)cc6c5c4c3o2)cc1 ; c1ccc(-c2nc3ccc4oc5ccc6ccc(-c7nc(-c8ccc(-n9c%10ccccc%10c%10ccccc%109)cc8)nc(-c8cccc9ccccc89)n7)cc6c5c4c3o2)cc1]

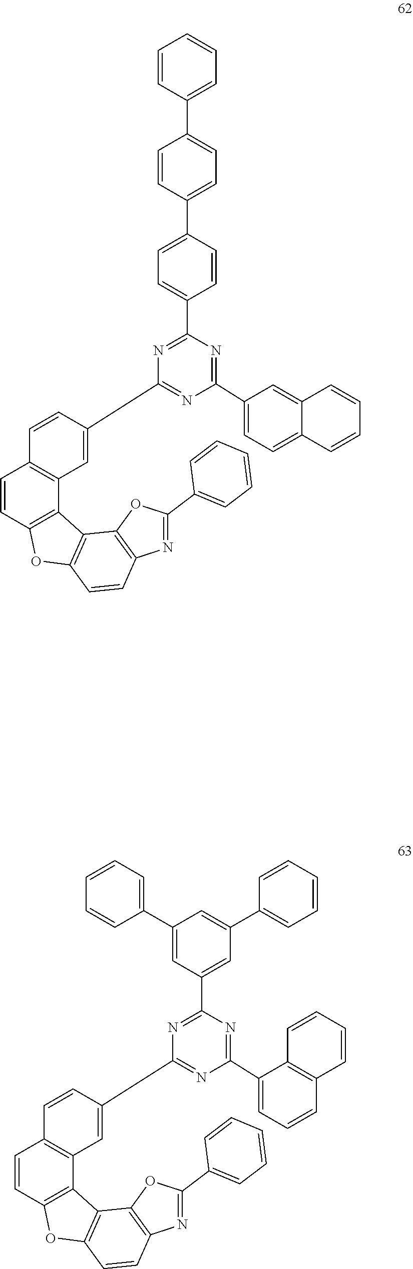 [SMILES: c1ccc(-c2ccc(-c3ccc(-c4nc(-c5ccc6ccccc6c5)nc(-c5ccc6ccc7oc8ccc9nc(-c%10ccccc%10)oc9c8c7c6c5)n4)cc3)cc2)cc1 ; c1ccc(-c2cc(-c3ccccc3)cc(-c3nc(-c4ccc5ccc6oc7ccc8nc(-c9ccccc9)oc8c7c6c5c4)nc(-c4cccc5ccccc45)n3)c2)cc1]

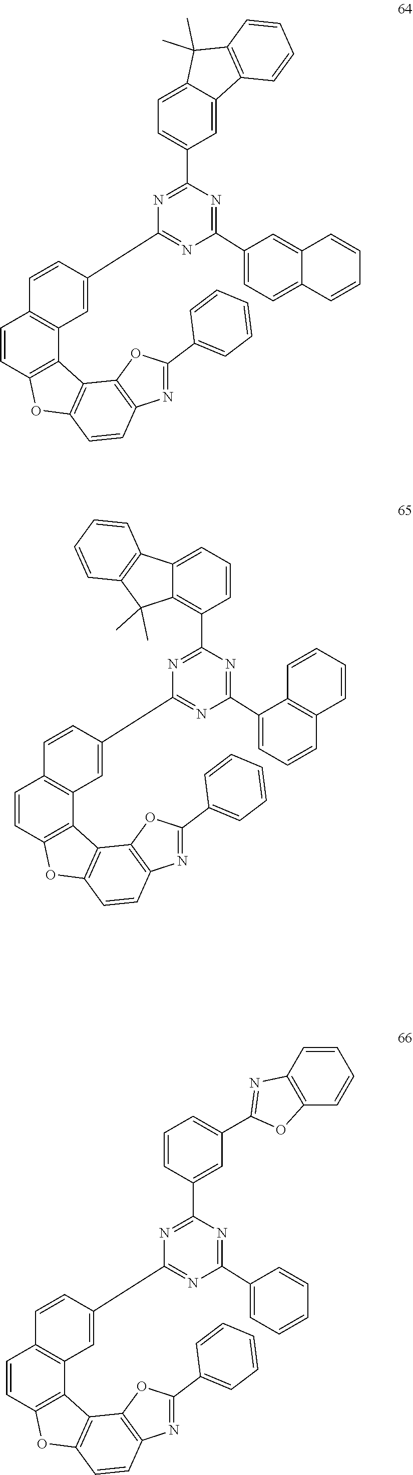 [SMILES: CC1(C)c2ccccc2-c2cc(-c3nc(-c4ccc5ccccc5c4)nc(-c4ccc5ccc6oc7ccc8nc(-c9ccccc9)oc8c7c6c5c4)n3)ccc21 ; CC1(C)c2ccccc2-c2cccc(-c3nc(-c4ccc5ccc6oc7ccc8nc(-c9ccccc9)oc8c7c6c5c4)nc(-c4cccc5ccccc45)n3)c21 ; c1ccc(-c2nc(-c3cccc(-c4nc5ccccc5o4)c3)nc(-c3ccc4ccc5oc6ccc7nc(-c8ccccc8)oc7c6c5c4c3)n2)cc1]

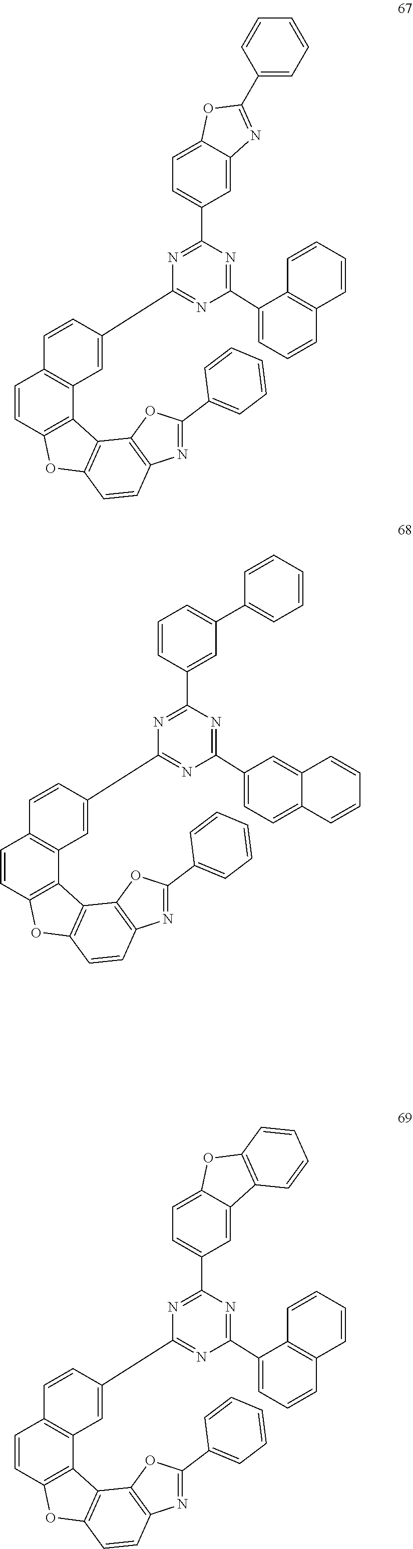 [SMILES: c1ccc(-c2nc3cc(-c4nc(-c5ccc6ccc7oc8ccc9nc(-c%10ccccc%10)oc9c8c7c6c5)nc(-c5cccc6ccccc56)n4)ccc3o2)cc1 ; c1ccc(-c2cccc(-c3nc(-c4ccc5ccccc5c4)nc(-c4ccc5ccc6oc7ccc8nc(-c9ccccc9)oc8c7c6c5c4)n3)c2)cc1 ; c1ccc(-c2nc3ccc4oc5ccc6ccc(-c7nc(-c8ccc9oc%10ccccc%10c9c8)nc(-c8cccc9ccccc89)n7)cc6c5c4c3o2)cc1]

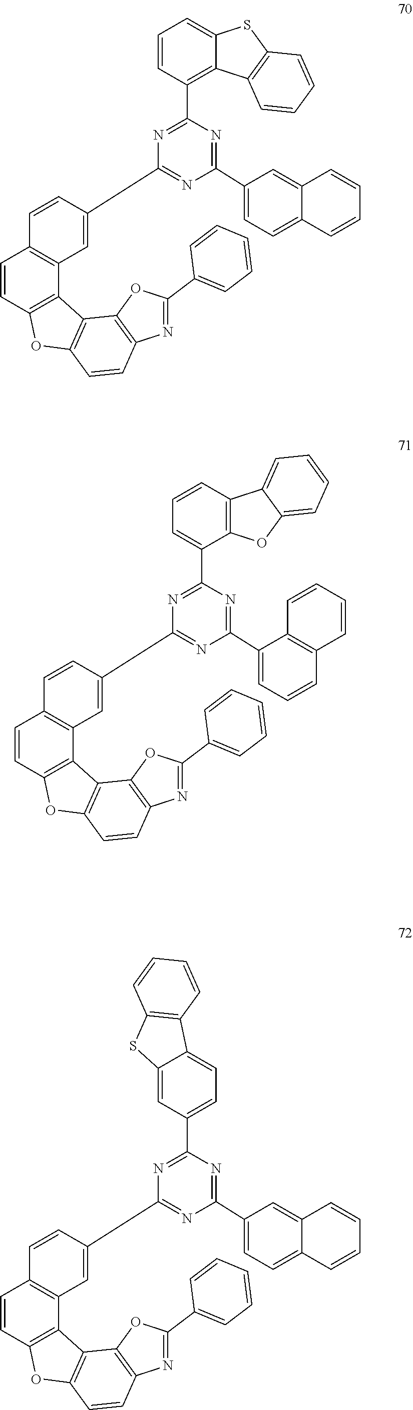 [SMILES: c1ccc(-c2nc3ccc4oc5ccc6ccc(-c7nc(-c8ccc9ccccc9c8)nc(-c8cccc9sc%10ccccc%10c89)n7)cc6c5c4c3o2)cc1 ; c1ccc(-c2nc3ccc4oc5ccc6ccc(-c7nc(-c8cccc9ccccc89)nc(-c8cccc9c8oc8ccccc89)n7)cc6c5c4c3o2)cc1 ; c1ccc(-c2nc3ccc4oc5ccc6ccc(-c7nc(-c8ccc9ccccc9c8)nc(-c8ccc9c(c8)sc8ccccc89)n7)cc6c5c4c3o2)cc1]

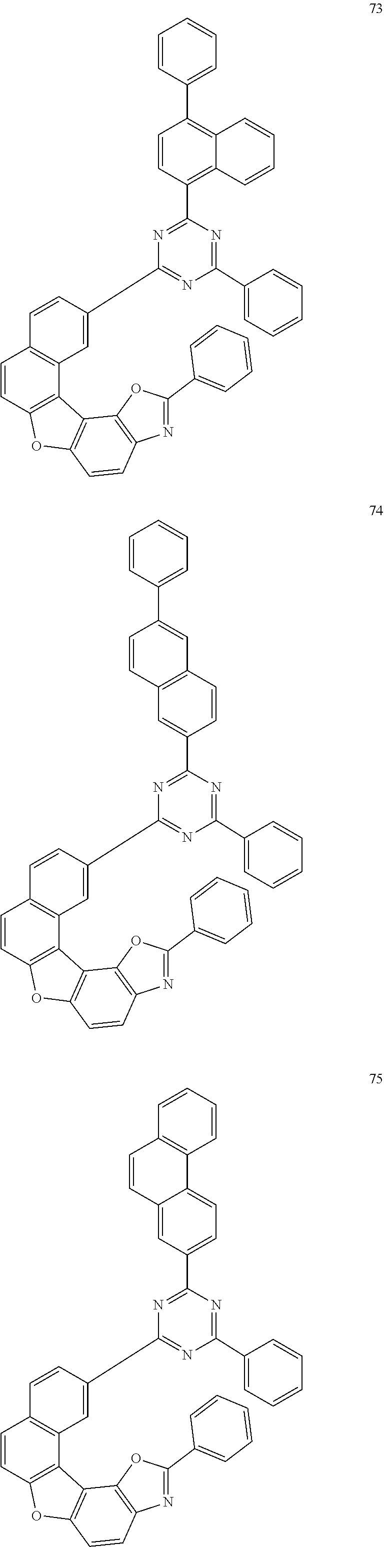 [SMILES: c1ccc(-c2nc(-c3ccc4ccc5oc6ccc7nc(-c8ccccc8)oc7c6c5c4c3)nc(-c3ccc(-c4ccccc4)c4ccccc34)n2)cc1 ; c1ccc(-c2ccc3cc(-c4nc(-c5ccccc5)nc(-c5ccc6ccc7oc8ccc9nc(-c%10ccccc%10)oc9c8c7c6c5)n4)ccc3c2)cc1 ; c1ccc(-c2nc(-c3ccc4c(ccc5ccccc54)c3)nc(-c3ccc4ccc5oc6ccc7nc(-c8ccccc8)oc7c6c5c4c3)n2)cc1]

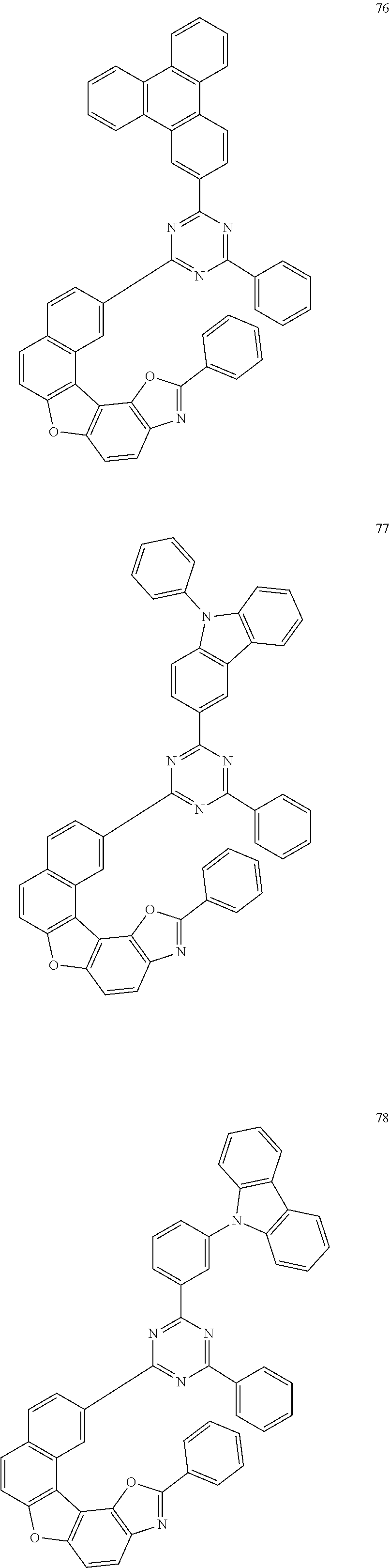 [SMILES: c1ccc(-c2nc(-c3ccc4c5ccccc5c5ccccc5c4c3)nc(-c3ccc4ccc5oc6ccc7nc(-c8ccccc8)oc7c6c5c4c3)n2)cc1 ; c1ccc(-c2nc(-c3ccc4ccc5oc6ccc7nc(-c8ccccc8)oc7c6c5c4c3)nc(-c3ccc4c(c3)c3ccccc3n4-c3ccccc3)n2)cc1 ; c1ccc(-c2nc(-c3cccc(-n4c5ccccc5c5ccccc54)c3)nc(-c3ccc4ccc5oc6ccc7nc(-c8ccccc8)oc7c6c5c4c3)n2)cc1]

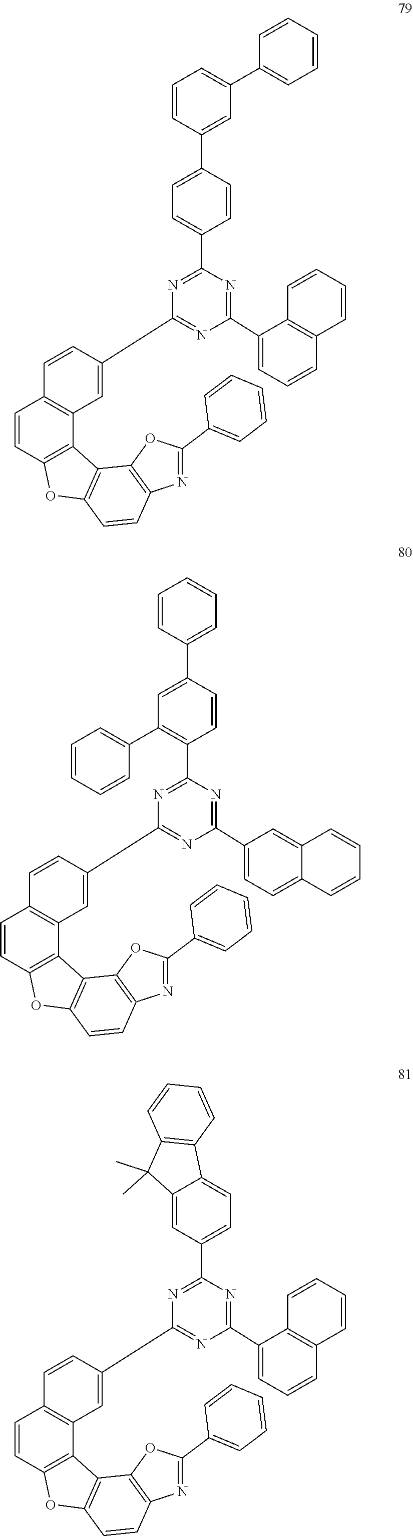 [SMILES: c1ccc(-c2cccc(-c3ccc(-c4nc(-c5ccc6ccc7oc8ccc9nc(-c%10ccccc%10)oc9c8c7c6c5)nc(-c5cccc6ccccc56)n4)cc3)c2)cc1 ; c1ccc(-c2ccc(-c3nc(-c4ccc5ccccc5c4)nc(-c4ccc5ccc6oc7ccc8nc(-c9ccccc9)oc8c7c6c5c4)n3)c(-c3ccccc3)c2)cc1 ; CC1(C)c2ccccc2-c2ccc(-c3nc(-c4ccc5ccc6oc7ccc8nc(-c9ccccc9)oc8c7c6c5c4)nc(-c4cccc5ccccc45)n3)cc21]

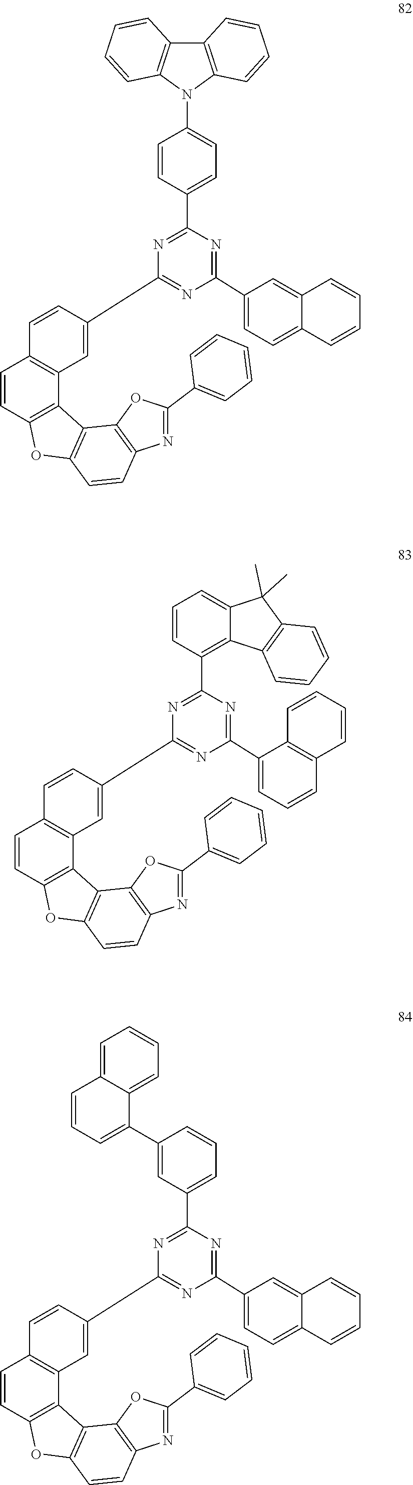 [SMILES: c1ccc(-c2nc3ccc4oc5ccc6ccc(-c7nc(-c8ccc(-n9c%10ccccc%10c%10ccccc%109)cc8)nc(-c8ccc9ccccc9c8)n7)cc6c5c4c3o2)cc1 ; CC1(C)c2ccccc2-c2c(-c3nc(-c4ccc5ccc6oc7ccc8nc(-c9ccccc9)oc8c7c6c5c4)nc(-c4cccc5ccccc45)n3)cccc21 ; c1ccc(-c2nc3ccc4oc5ccc6ccc(-c7nc(-c8cccc(-c9cccc%10ccccc9%10)c8)nc(-c8ccc9ccccc9c8)n7)cc6c5c4c3o2)cc1]

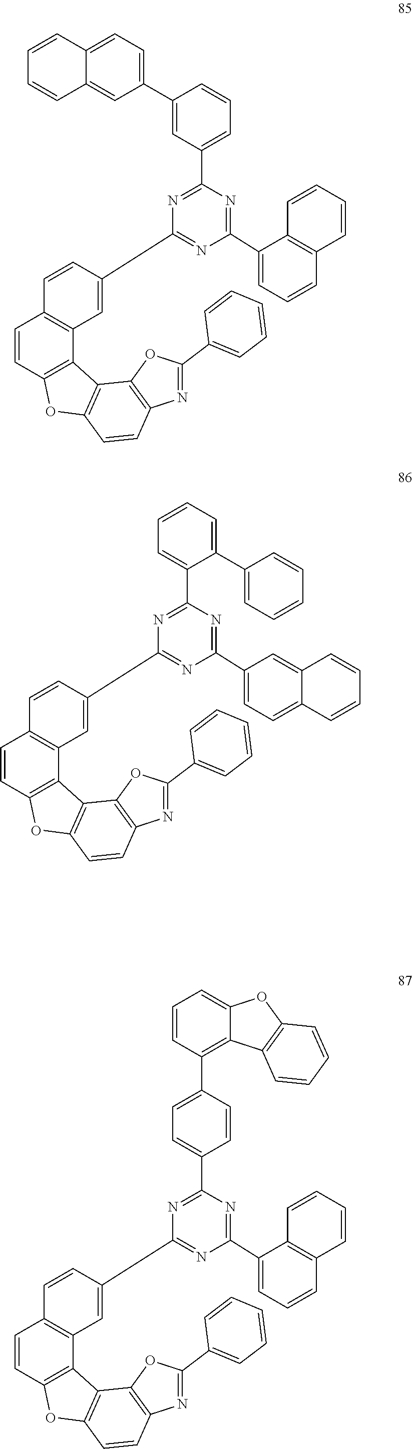 [SMILES: c1ccc(-c2nc3ccc4oc5ccc6ccc(-c7nc(-c8cccc(-c9ccc%10ccccc%10c9)c8)nc(-c8cccc9ccccc89)n7)cc6c5c4c3o2)cc1 ; c1ccc(-c2nc3ccc4oc5ccc6ccc(-c7nc(-c8ccc9ccccc9c8)nc(-c8ccccc8-c8ccccc8)n7)cc6c5c4c3o2)cc1 ; c1ccc(-c2nc3ccc4oc5ccc6ccc(-c7nc(-c8ccc(-c9cccc%10oc%11ccccc%11c9%10)cc8)nc(-c8cccc9ccccc89)n7)cc6c5c4c3o2)cc1]

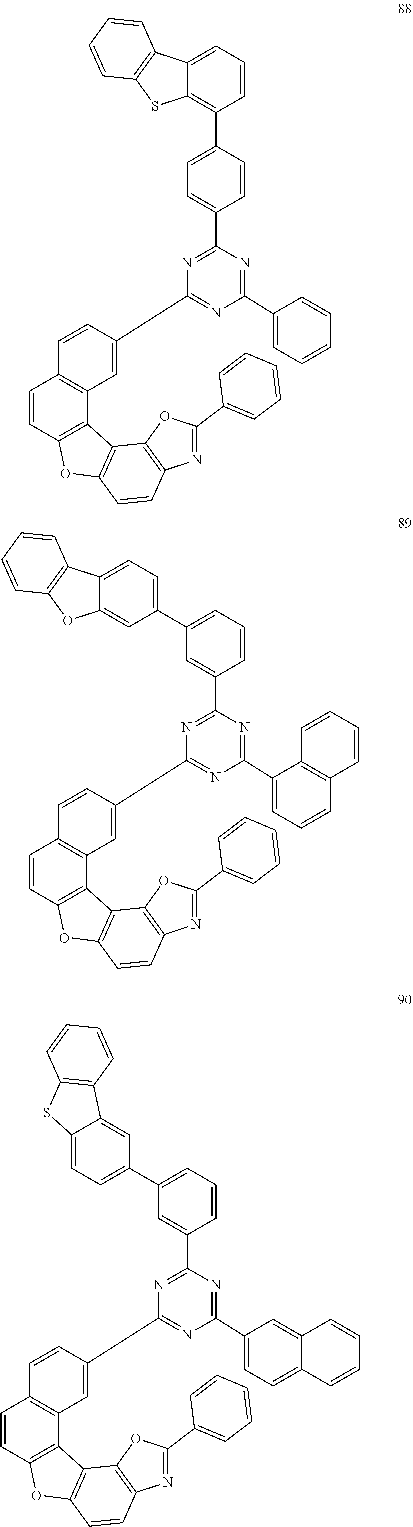 [SMILES: c1ccc(-c2nc(-c3ccc(-c4cccc5c4sc4ccccc45)cc3)nc(-c3ccc4ccc5oc6ccc7nc(-c8ccccc8)oc7c6c5c4c3)n2)cc1 ; c1ccc(-c2nc3ccc4oc5ccc6ccc(-c7nc(-c8cccc(-c9ccc%10c(c9)oc9ccccc9%10)c8)nc(-c8cccc9ccccc89)n7)cc6c5c4c3o2)cc1 ; c1ccc(-c2nc3ccc4oc5ccc6ccc(-c7nc(-c8cccc(-c9ccc%10sc%11ccccc%11c%10c9)c8)nc(-c8ccc9ccccc9c8)n7)cc6c5c4c3o2)cc1]

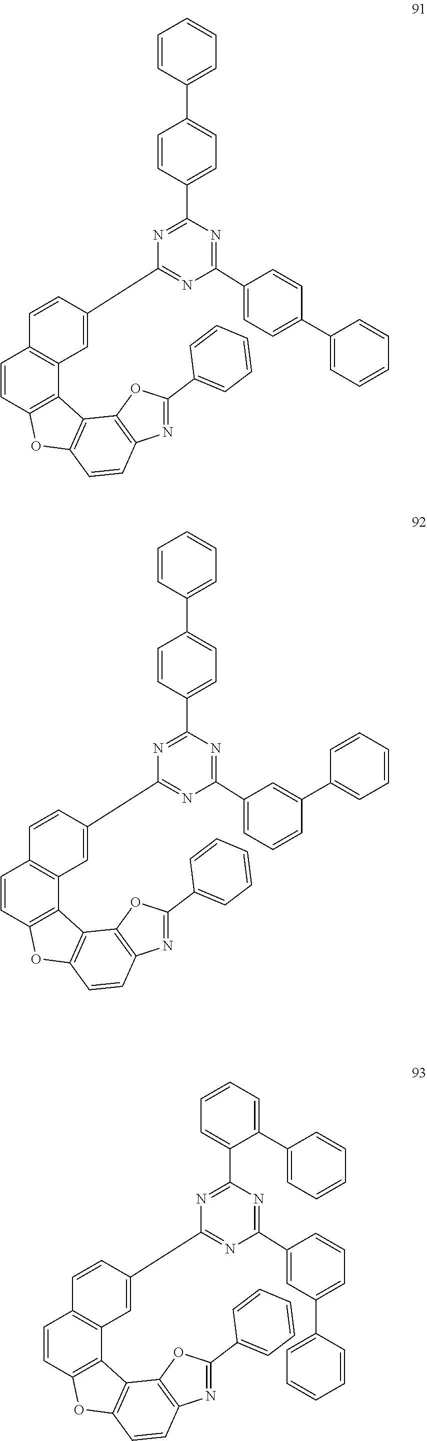 [SMILES: c1ccc(-c2ccc(-c3nc(-c4ccc(-c5ccccc5)cc4)nc(-c4ccc5ccc6oc7ccc8nc(-c9ccccc9)oc8c7c6c5c4)n3)cc2)cc1 ; c1ccc(-c2ccc(-c3nc(-c4cccc(-c5ccccc5)c4)nc(-c4ccc5ccc6oc7ccc8nc(-c9ccccc9)oc8c7c6c5c4)n3)cc2)cc1 ; c1ccc(-c2cccc(-c3nc(-c4ccc5ccc6oc7ccc8nc(-c9ccccc9)oc8c7c6c5c4)nc(-c4ccccc4-c4ccccc4)n3)c2)cc1]

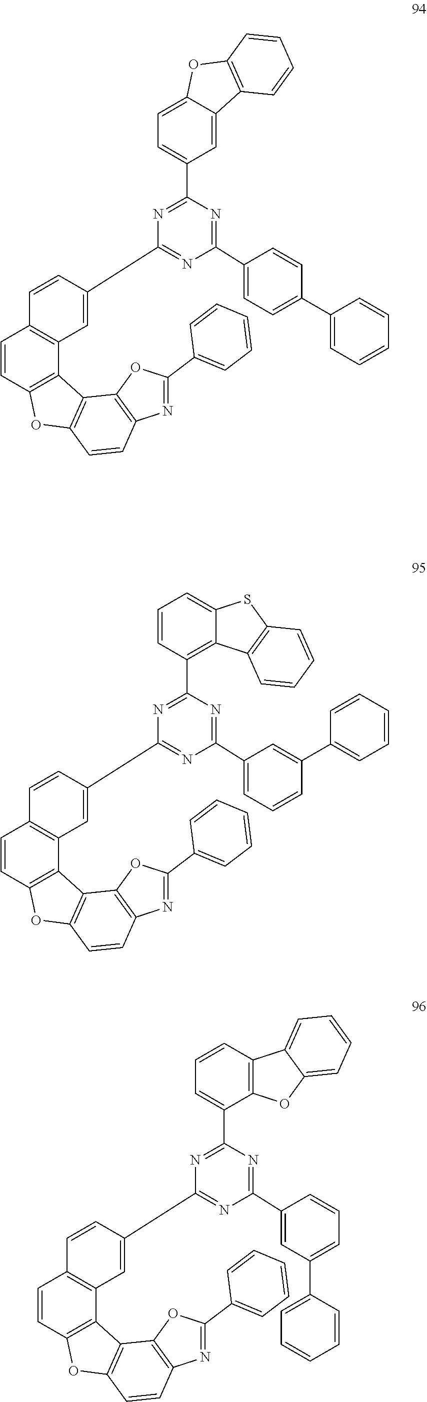 [SMILES: c1ccc(-c2ccc(-c3nc(-c4ccc5oc6ccccc6c5c4)nc(-c4ccc5ccc6oc7ccc8nc(-c9ccccc9)oc8c7c6c5c4)n3)cc2)cc1 ; c1ccc(-c2cccc(-c3nc(-c4ccc5ccc6oc7ccc8nc(-c9ccccc9)oc8c7c6c5c4)nc(-c4cccc5sc6ccccc6c45)n3)c2)cc1 ; c1ccc(-c2cccc(-c3nc(-c4ccc5ccc6oc7ccc8nc(-c9ccccc9)oc8c7c6c5c4)nc(-c4cccc5c4oc4ccccc45)n3)c2)cc1]

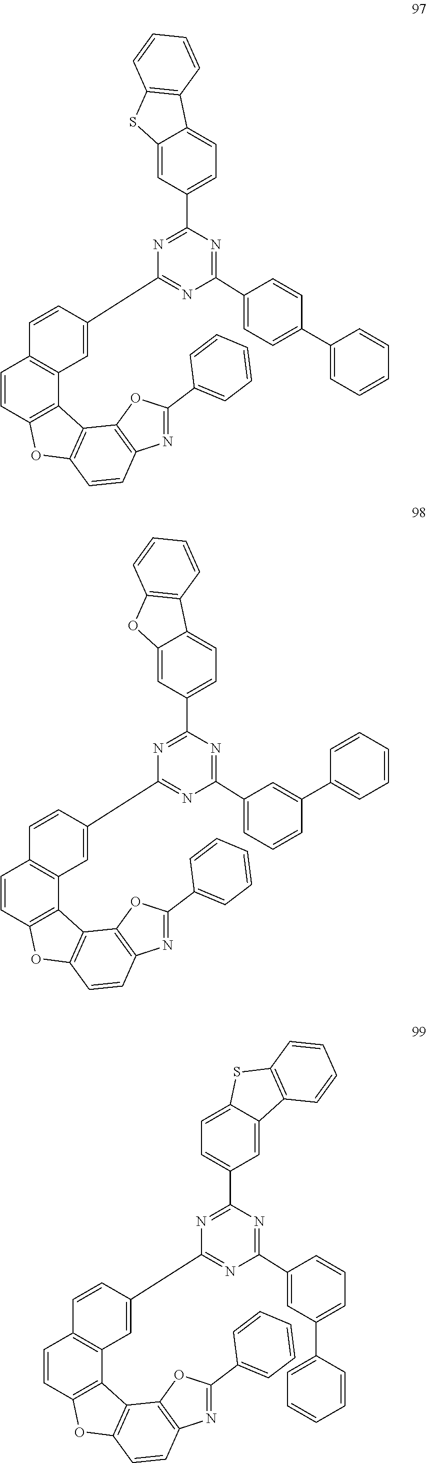 [SMILES: c1ccc(-c2ccc(-c3nc(-c4ccc5c(c4)sc4ccccc45)nc(-c4ccc5ccc6oc7ccc8nc(-c9ccccc9)oc8c7c6c5c4)n3)cc2)cc1 ; c1ccc(-c2cccc(-c3nc(-c4ccc5c(c4)oc4ccccc45)nc(-c4ccc5ccc6oc7ccc8nc(-c9ccccc9)oc8c7c6c5c4)n3)c2)cc1 ; c1ccc(-c2cccc(-c3nc(-c4ccc5sc6ccccc6c5c4)nc(-c4ccc5ccc6oc7ccc8nc(-c9ccccc9)oc8c7c6c5c4)n3)c2)cc1]

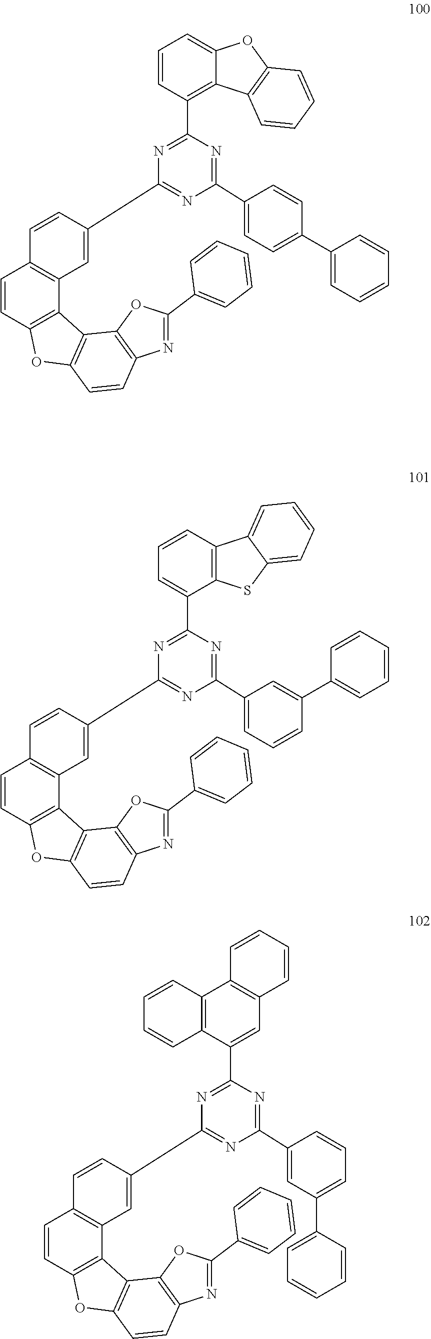 [SMILES: c1ccc(-c2ccc(-c3nc(-c4ccc5ccc6oc7ccc8nc(-c9ccccc9)oc8c7c6c5c4)nc(-c4cccc5oc6ccccc6c45)n3)cc2)cc1 ; c1ccc(-c2cccc(-c3nc(-c4ccc5ccc6oc7ccc8nc(-c9ccccc9)oc8c7c6c5c4)nc(-c4cccc5c4sc4ccccc45)n3)c2)cc1 ; c1ccc(-c2cccc(-c3nc(-c4ccc5ccc6oc7ccc8nc(-c9ccccc9)oc8c7c6c5c4)nc(-c4cc5ccccc5c5ccccc45)n3)c2)cc1]

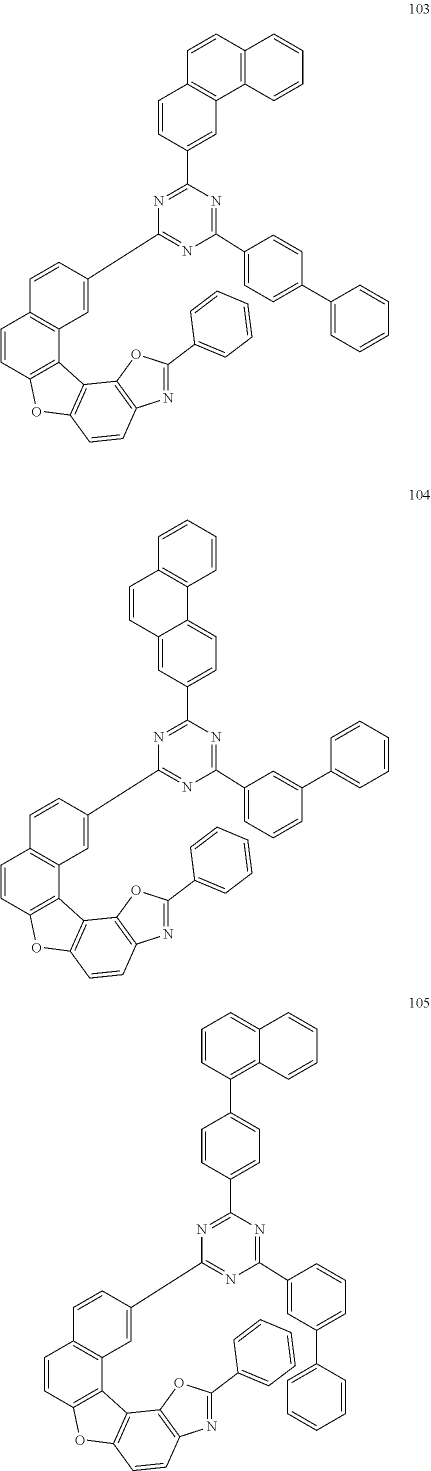 [SMILES: c1ccc(-c2ccc(-c3nc(-c4ccc5ccc6ccccc6c5c4)nc(-c4ccc5ccc6oc7ccc8nc(-c9ccccc9)oc8c7c6c5c4)n3)cc2)cc1 ; c1ccc(-c2cccc(-c3nc(-c4ccc5c(ccc6ccccc65)c4)nc(-c4ccc5ccc6oc7ccc8nc(-c9ccccc9)oc8c7c6c5c4)n3)c2)cc1 ; c1ccc(-c2cccc(-c3nc(-c4ccc(-c5cccc6ccccc56)cc4)nc(-c4ccc5ccc6oc7ccc8nc(-c9ccccc9)oc8c7c6c5c4)n3)c2)cc1]

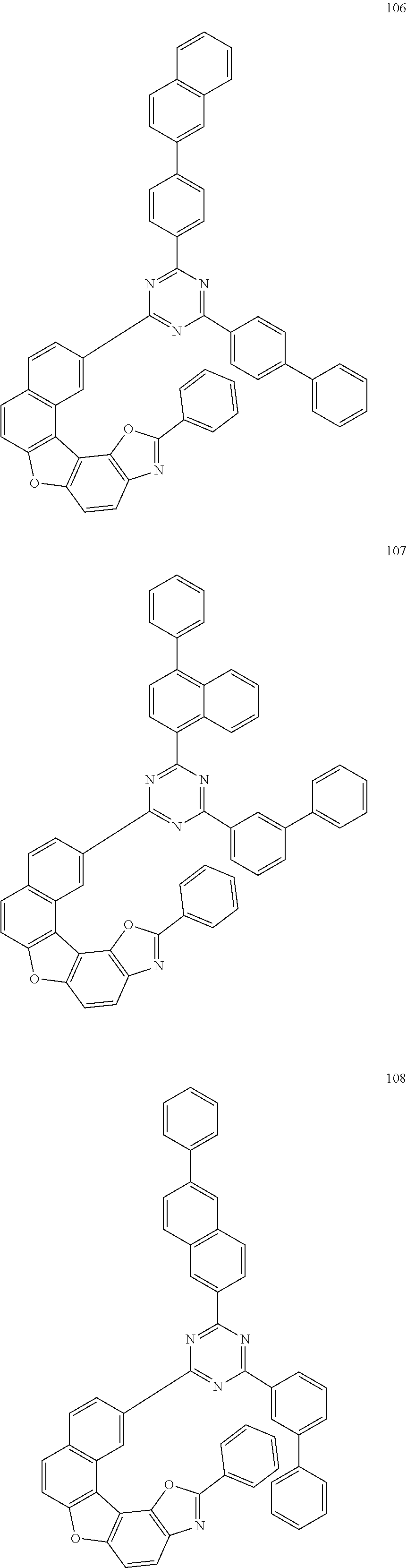 [SMILES: c1ccc(-c2ccc(-c3nc(-c4ccc(-c5ccc6ccccc6c5)cc4)nc(-c4ccc5ccc6oc7ccc8nc(-c9ccccc9)oc8c7c6c5c4)n3)cc2)cc1 ; c1ccc(-c2cccc(-c3nc(-c4ccc5ccc6oc7ccc8nc(-c9ccccc9)oc8c7c6c5c4)nc(-c4ccc(-c5ccccc5)c5ccccc45)n3)c2)cc1 ; c1ccc(-c2cccc(-c3nc(-c4ccc5cc(-c6ccccc6)ccc5c4)nc(-c4ccc5ccc6oc7ccc8nc(-c9ccccc9)oc8c7c6c5c4)n3)c2)cc1]

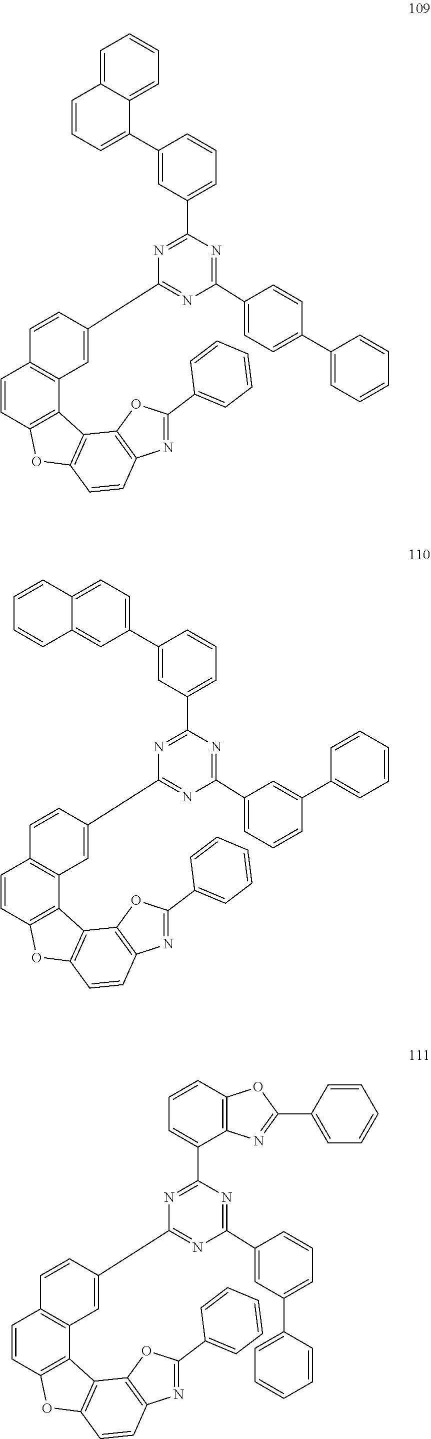 [SMILES: c1ccc(-c2ccc(-c3nc(-c4cccc(-c5cccc6ccccc56)c4)nc(-c4ccc5ccc6oc7ccc8nc(-c9ccccc9)oc8c7c6c5c4)n3)cc2)cc1 ; c1ccc(-c2cccc(-c3nc(-c4cccc(-c5ccc6ccccc6c5)c4)nc(-c4ccc5ccc6oc7ccc8nc(-c9ccccc9)oc8c7c6c5c4)n3)c2)cc1 ; c1ccc(-c2cccc(-c3nc(-c4ccc5ccc6oc7ccc8nc(-c9ccccc9)oc8c7c6c5c4)nc(-c4cccc5oc(-c6ccccc6)nc45)n3)c2)cc1]

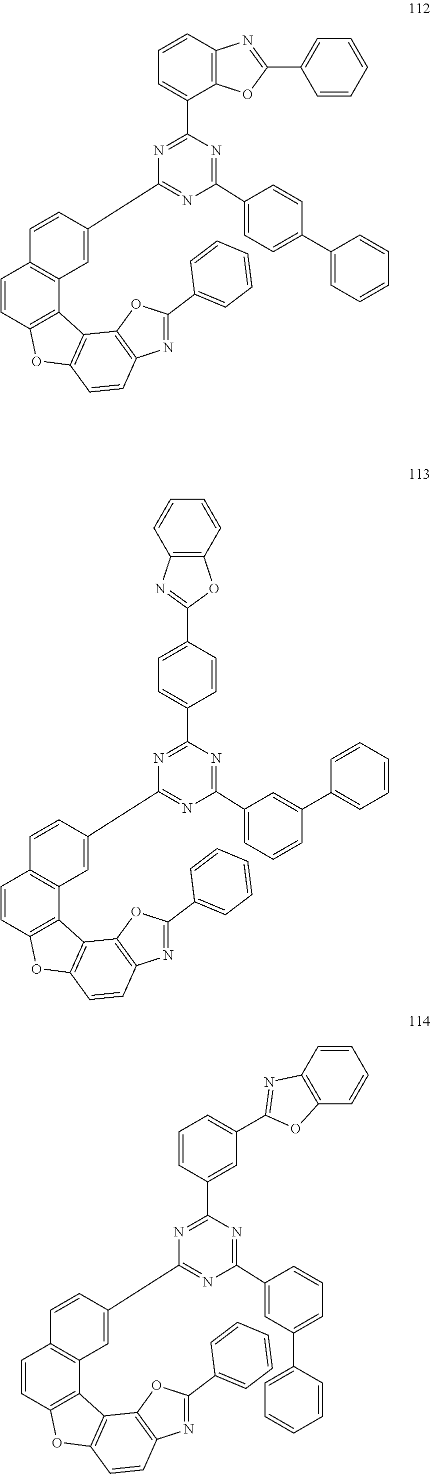 [SMILES: c1ccc(-c2ccc(-c3nc(-c4ccc5ccc6oc7ccc8nc(-c9ccccc9)oc8c7c6c5c4)nc(-c4cccc5nc(-c6ccccc6)oc45)n3)cc2)cc1 ; c1ccc(-c2cccc(-c3nc(-c4ccc(-c5nc6ccccc6o5)cc4)nc(-c4ccc5ccc6oc7ccc8nc(-c9ccccc9)oc8c7c6c5c4)n3)c2)cc1 ; c1ccc(-c2cccc(-c3nc(-c4cccc(-c5nc6ccccc6o5)c4)nc(-c4ccc5ccc6oc7ccc8nc(-c9ccccc9)oc8c7c6c5c4)n3)c2)cc1]

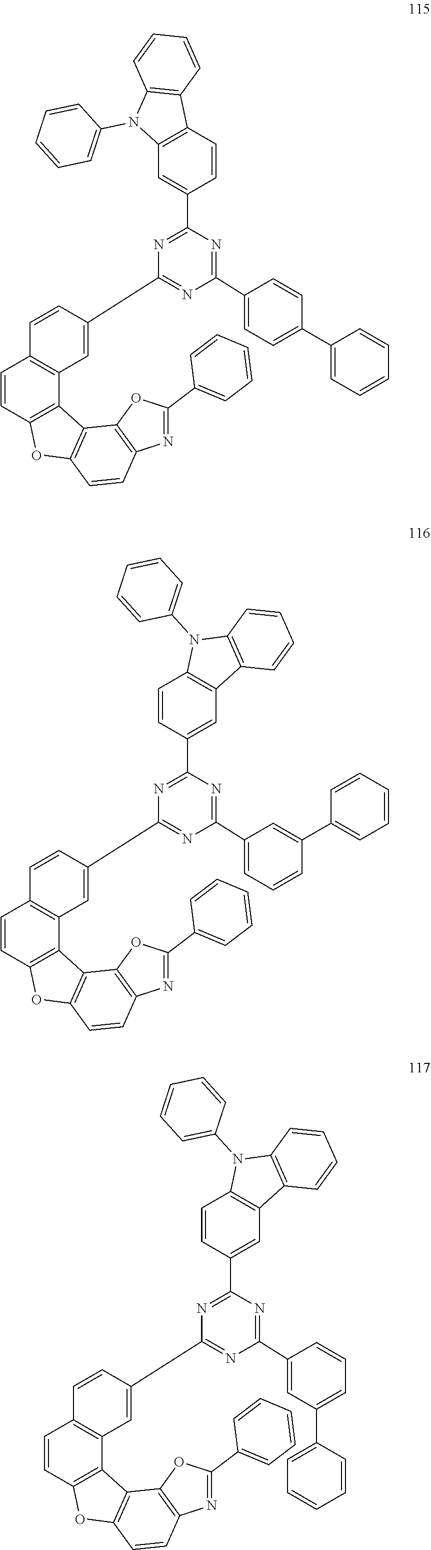 [SMILES: c1ccc(-c2ccc(-c3nc(-c4ccc5ccc6oc7ccc8nc(-c9ccccc9)oc8c7c6c5c4)nc(-c4ccc5c6ccccc6n(-c6ccccc6)c5c4)n3)cc2)cc1 ; c1ccc(-c2cccc(-c3nc(-c4ccc5ccc6oc7ccc8nc(-c9ccccc9)oc8c7c6c5c4)nc(-c4ccc5c(c4)c4ccccc4n5-c4ccccc4)n3)c2)cc1 ; c1ccc(-c2cccc(-c3nc(-c4ccc5ccc6oc7ccc8nc(-c9ccccc9)oc8c7c6c5c4)nc(-c4ccc5c(c4)c4ccccc4n5-c4ccccc4)n3)c2)cc1]

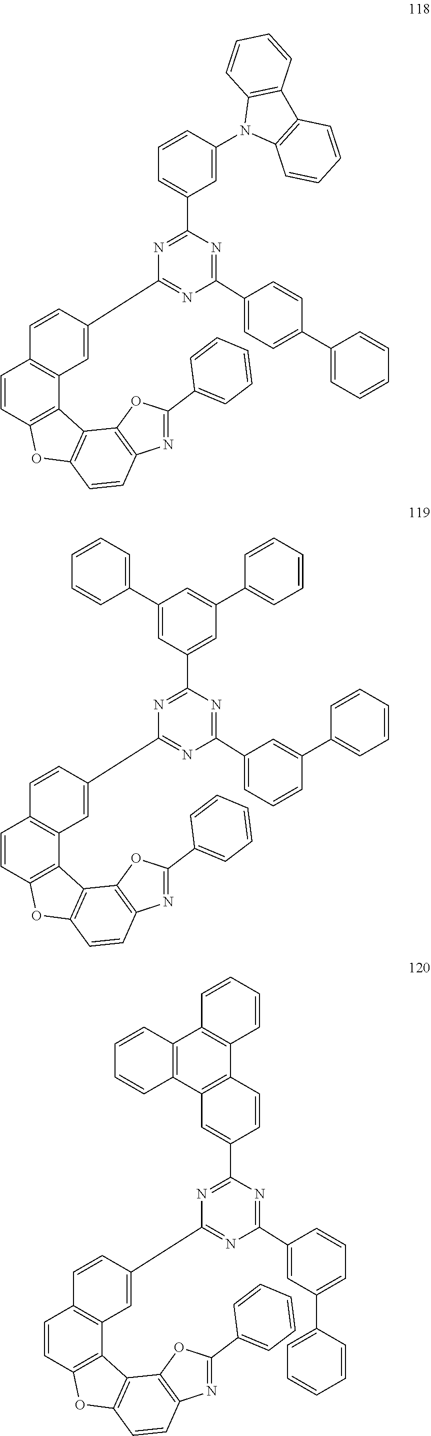 [SMILES: c1ccc(-c2ccc(-c3nc(-c4cccc(-n5c6ccccc6c6ccccc65)c4)nc(-c4ccc5ccc6oc7ccc8nc(-c9ccccc9)oc8c7c6c5c4)n3)cc2)cc1 ; c1ccc(-c2cccc(-c3nc(-c4cc(-c5ccccc5)cc(-c5ccccc5)c4)nc(-c4ccc5ccc6oc7ccc8nc(-c9ccccc9)oc8c7c6c5c4)n3)c2)cc1 ; c1ccc(-c2cccc(-c3nc(-c4ccc5c6ccccc6c6ccccc6c5c4)nc(-c4ccc5ccc6oc7ccc8nc(-c9ccccc9)oc8c7c6c5c4)n3)c2)cc1]

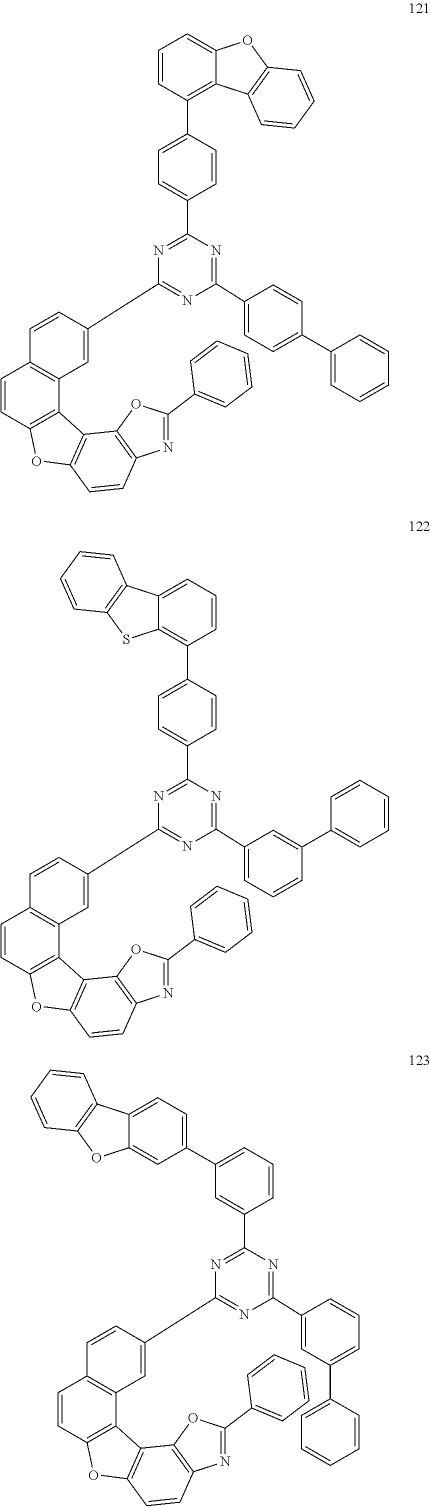

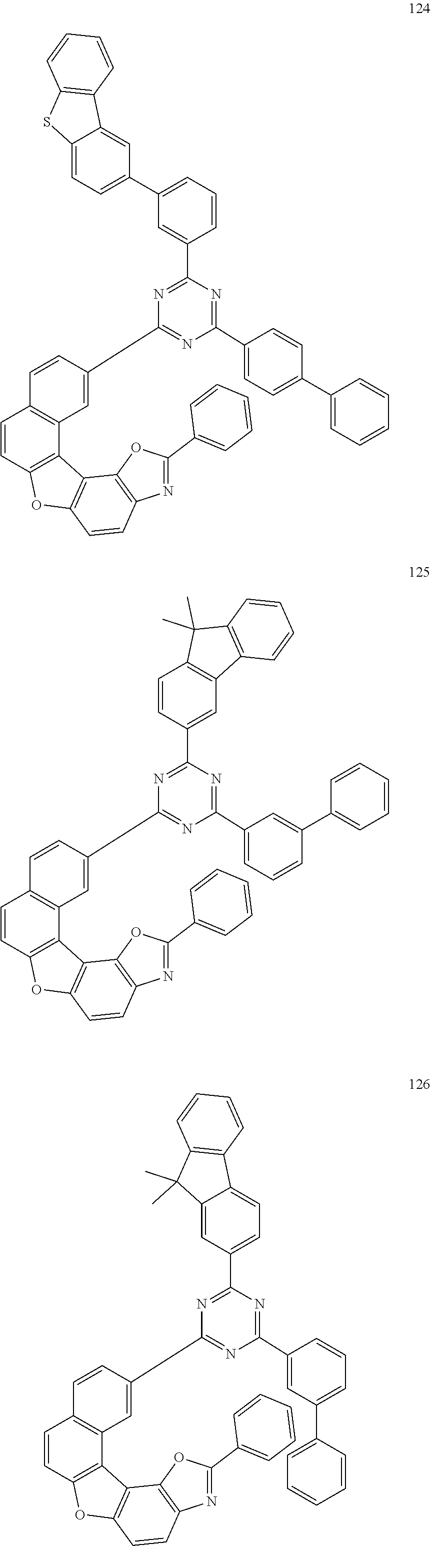 [SMILES: c1ccc(-c2ccc(-c3nc(-c4cccc(-c5ccc6sc7ccccc7c6c5)c4)nc(-c4ccc5ccc6oc7ccc8nc(-c9ccccc9)oc8c7c6c5c4)n3)cc2)cc1 ; CC1(C)c2ccccc2-c2cc(-c3nc(-c4cccc(-c5ccccc5)c4)nc(-c4ccc5ccc6oc7ccc8nc(-c9ccccc9)oc8c7c6c5c4)n3)ccc21 ; CC1(C)c2ccccc2-c2ccc(-c3nc(-c4cccc(-c5ccccc5)c4)nc(-c4ccc5ccc6oc7ccc8nc(-c9ccccc9)oc8c7c6c5c4)n3)cc21]

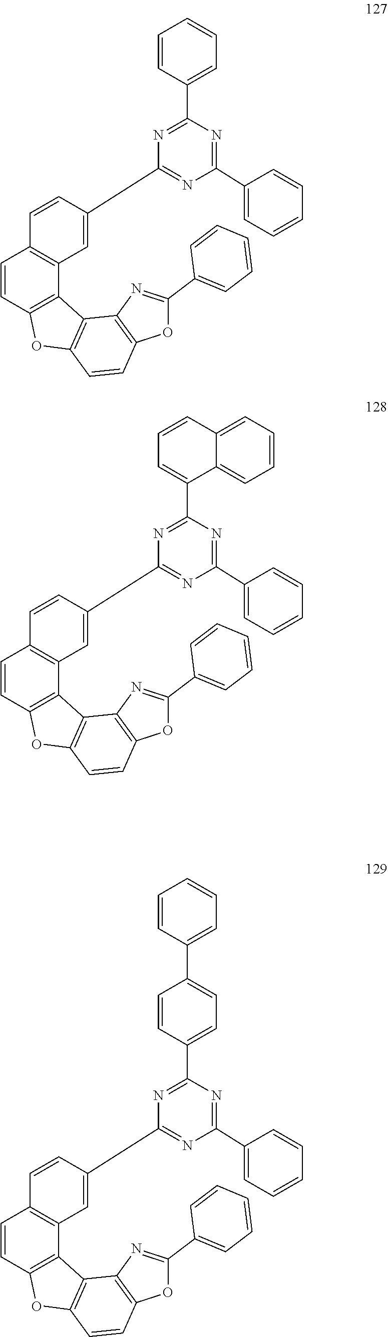 [SMILES: c1ccc(-c2nc(-c3ccccc3)nc(-c3ccc4ccc5oc6ccc7oc(-c8ccccc8)nc7c6c5c4c3)n2)cc1 ; c1ccc(-c2nc(-c3ccc4ccc5oc6ccc7oc(-c8ccccc8)nc7c6c5c4c3)nc(-c3cccc4ccccc34)n2)cc1 ; c1ccc(-c2ccc(-c3nc(-c4ccccc4)nc(-c4ccc5ccc6oc7ccc8oc(-c9ccccc9)nc8c7c6c5c4)n3)cc2)cc1]

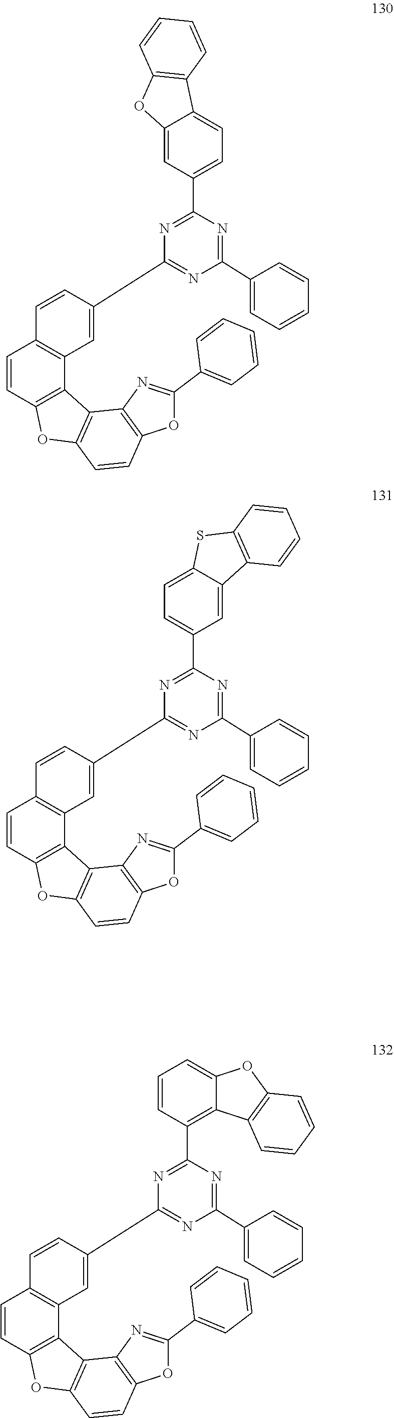 [SMILES: c1ccc(-c2nc(-c3ccc4c(c3)oc3ccccc34)nc(-c3ccc4ccc5oc6ccc7oc(-c8ccccc8)nc7c6c5c4c3)n2)cc1 ; c1ccc(-c2nc(-c3ccc4sc5ccccc5c4c3)nc(-c3ccc4ccc5oc6ccc7oc(-c8ccccc8)nc7c6c5c4c3)n2)cc1 ; c1ccc(-c2nc(-c3ccc4ccc5oc6ccc7oc(-c8ccccc8)nc7c6c5c4c3)nc(-c3cccc4oc5ccccc5c34)n2)cc1]

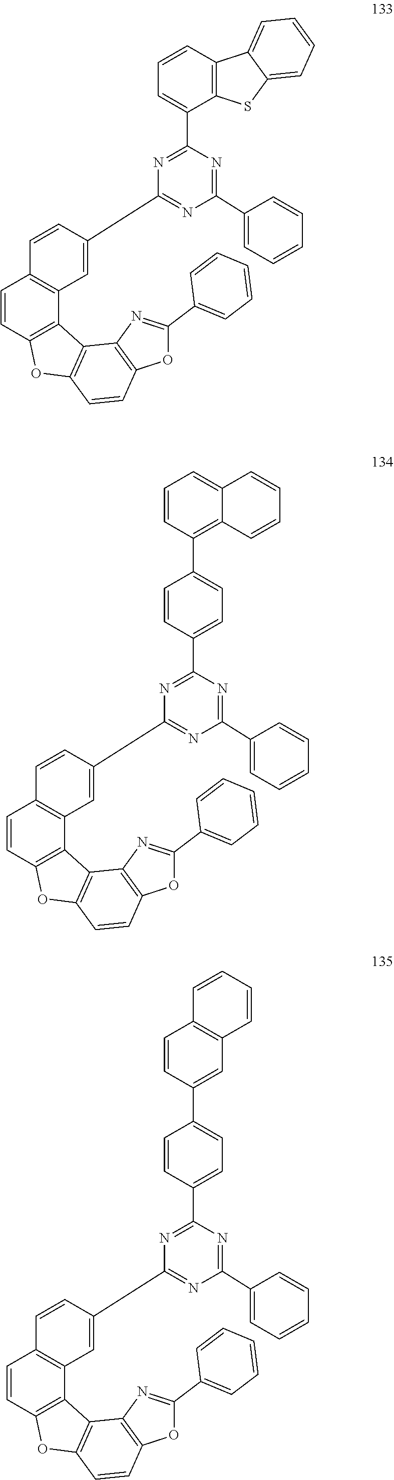 [SMILES: c1ccc(-c2nc(-c3ccc4ccc5oc6ccc7oc(-c8ccccc8)nc7c6c5c4c3)nc(-c3cccc4c3sc3ccccc34)n2)cc1 ; c1ccc(-c2nc(-c3ccc(-c4cccc5ccccc45)cc3)nc(-c3ccc4ccc5oc6ccc7oc(-c8ccccc8)nc7c6c5c4c3)n2)cc1 ; c1ccc(-c2nc(-c3ccc(-c4ccc5ccccc5c4)cc3)nc(-c3ccc4ccc5oc6ccc7oc(-c8ccccc8)nc7c6c5c4c3)n2)cc1]

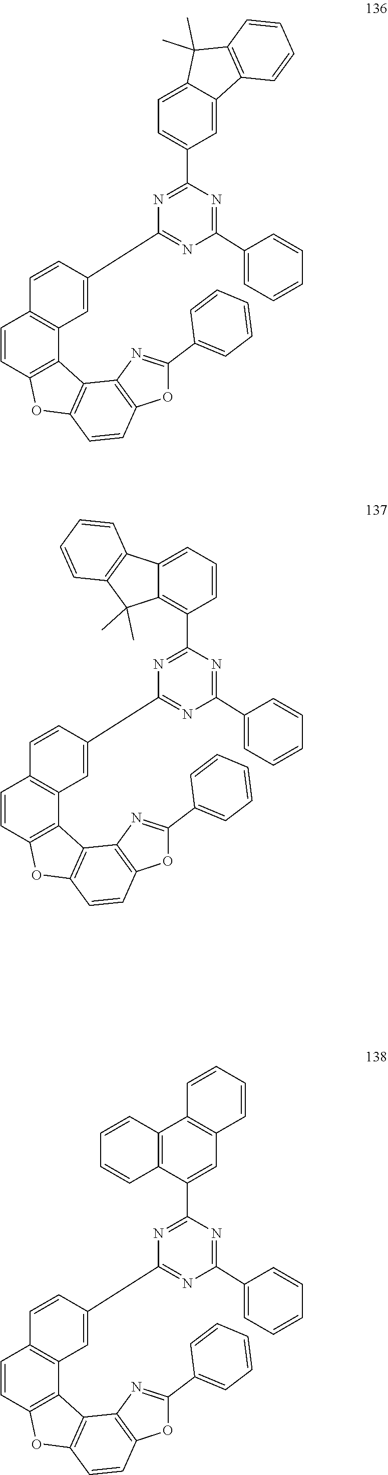 [SMILES: CC1(C)c2ccccc2-c2cc(-c3nc(-c4ccccc4)nc(-c4ccc5ccc6oc7ccc8oc(-c9ccccc9)nc8c7c6c5c4)n3)ccc21 ; CC1(C)c2ccccc2-c2cccc(-c3nc(-c4ccccc4)nc(-c4ccc5ccc6oc7ccc8oc(-c9ccccc9)nc8c7c6c5c4)n3)c21 ; c1ccc(-c2nc(-c3ccc4ccc5oc6ccc7oc(-c8ccccc8)nc7c6c5c4c3)nc(-c3cc4ccccc4c4ccccc34)n2)cc1]

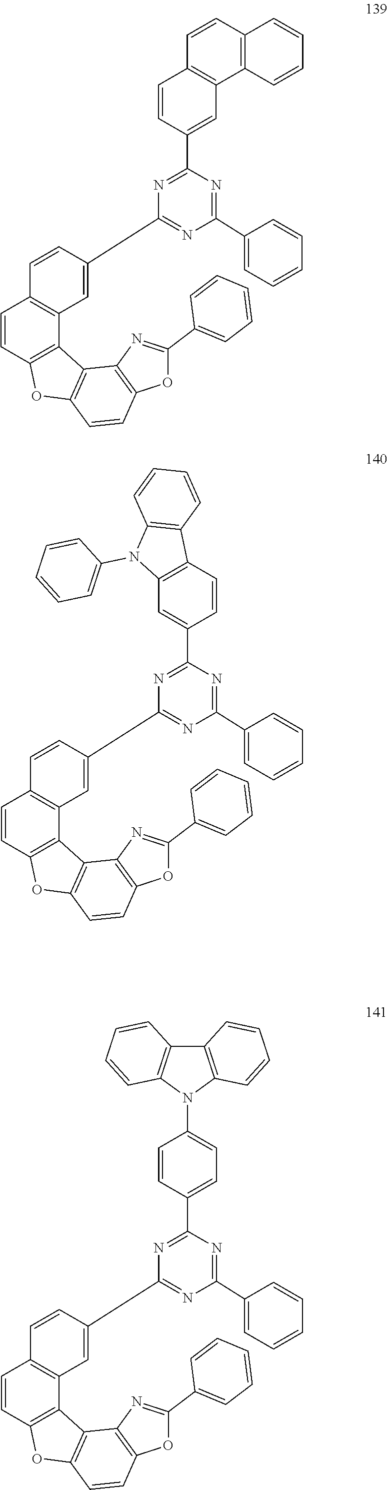 [SMILES: c1ccc(-c2nc(-c3ccc4ccc5ccccc5c4c3)nc(-c3ccc4ccc5oc6ccc7oc(-c8ccccc8)nc7c6c5c4c3)n2)cc1 ; c1ccc(-c2nc(-c3ccc4ccc5oc6ccc7oc(-c8ccccc8)nc7c6c5c4c3)nc(-c3ccc4c5ccccc5n(-c5ccccc5)c4c3)n2)cc1 ; c1ccc(-c2nc(-c3ccc(-n4c5ccccc5c5ccccc54)cc3)nc(-c3ccc4ccc5oc6ccc7oc(-c8ccccc8)nc7c6c5c4c3)n2)cc1]

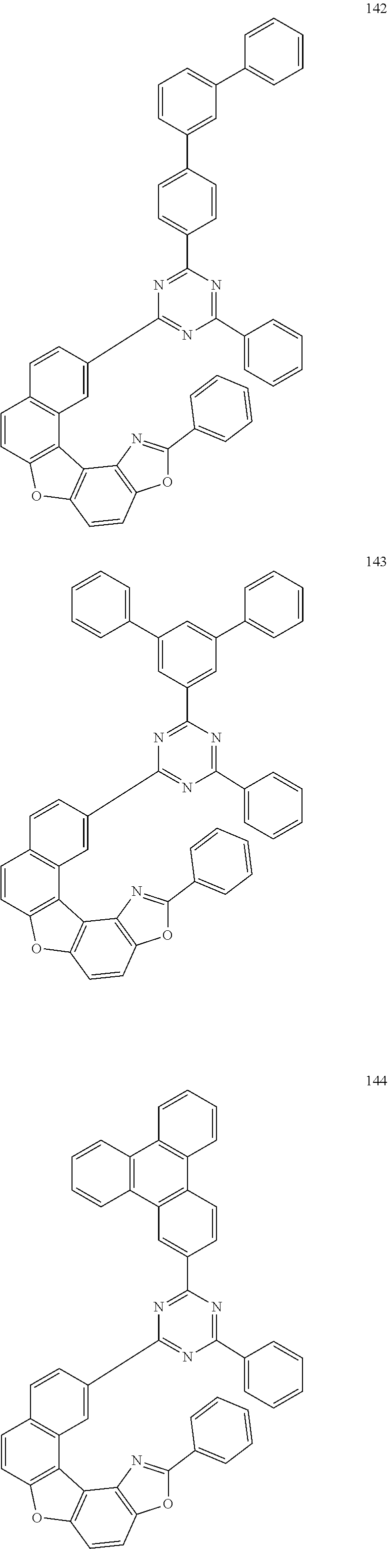

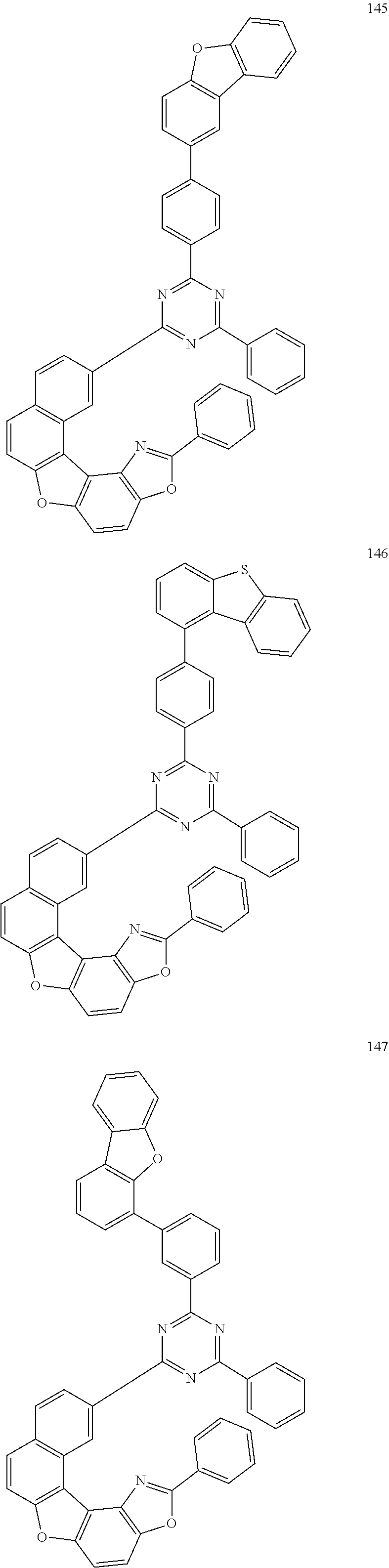 [SMILES: c1ccc(-c2nc(-c3ccc(-c4ccc5oc6ccccc6c5c4)cc3)nc(-c3ccc4ccc5oc6ccc7oc(-c8ccccc8)nc7c6c5c4c3)n2)cc1 ; c1ccc(-c2nc(-c3ccc(-c4cccc5sc6ccccc6c45)cc3)nc(-c3ccc4ccc5oc6ccc7oc(-c8ccccc8)nc7c6c5c4c3)n2)cc1 ; c1ccc(-c2nc(-c3cccc(-c4cccc5c4oc4ccccc45)c3)nc(-c3ccc4ccc5oc6ccc7oc(-c8ccccc8)nc7c6c5c4c3)n2)cc1]

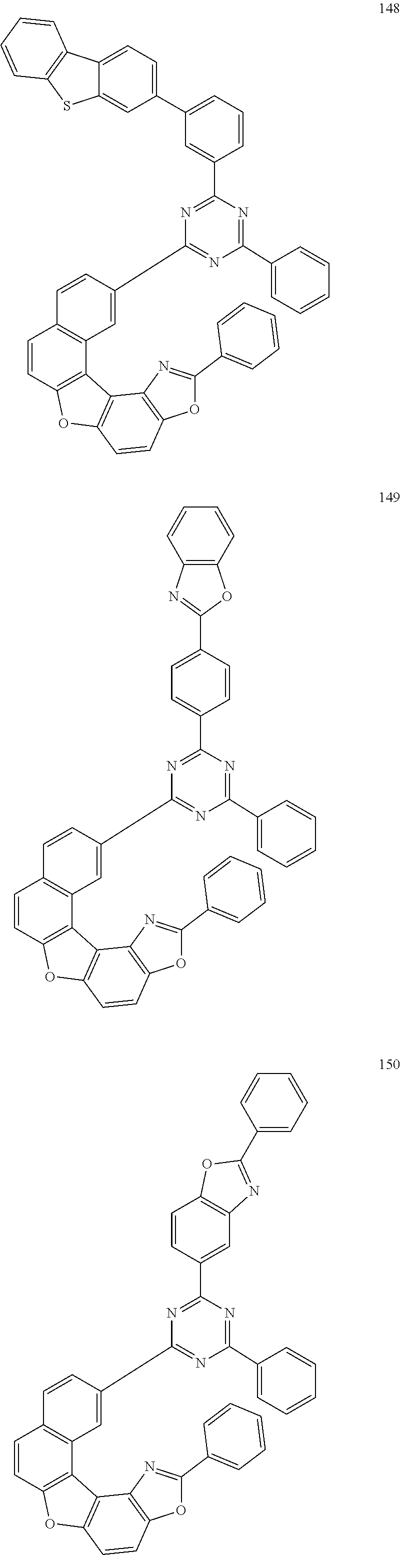 [SMILES: c1ccc(-c2nc(-c3cccc(-c4ccc5c(c4)sc4ccccc45)c3)nc(-c3ccc4ccc5oc6ccc7oc(-c8ccccc8)nc7c6c5c4c3)n2)cc1 ; c1ccc(-c2nc(-c3ccc(-c4nc5ccccc5o4)cc3)nc(-c3ccc4ccc5oc6ccc7oc(-c8ccccc8)nc7c6c5c4c3)n2)cc1 ; c1ccc(-c2nc(-c3ccc4oc(-c5ccccc5)nc4c3)nc(-c3ccc4ccc5oc6ccc7oc(-c8ccccc8)nc7c6c5c4c3)n2)cc1]

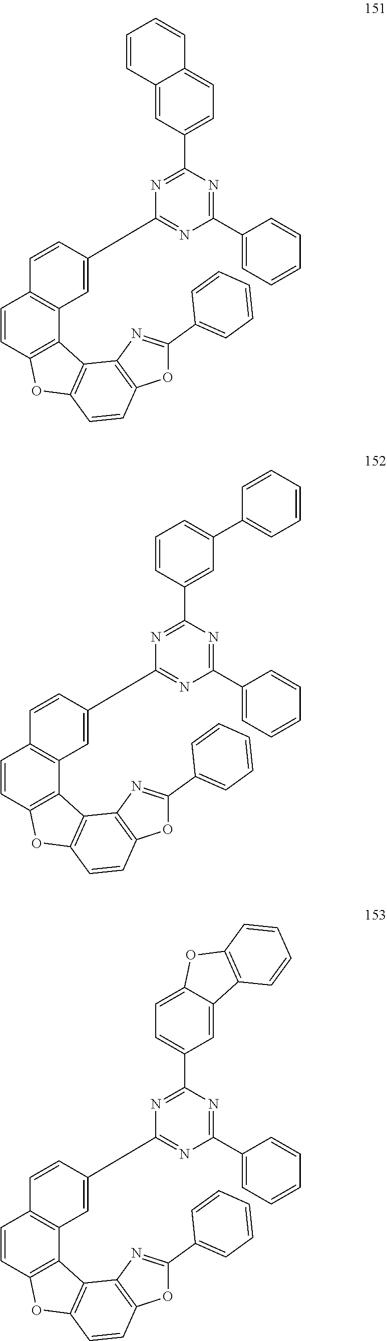 [SMILES: c1ccc(-c2nc(-c3ccc4ccccc4c3)nc(-c3ccc4ccc5oc6ccc7oc(-c8ccccc8)nc7c6c5c4c3)n2)cc1 ; c1ccc(-c2cccc(-c3nc(-c4ccccc4)nc(-c4ccc5ccc6oc7ccc8oc(-c9ccccc9)nc8c7c6c5c4)n3)c2)cc1 ; c1ccc(-c2nc(-c3ccc4oc5ccccc5c4c3)nc(-c3ccc4ccc5oc6ccc7oc(-c8ccccc8)nc7c6c5c4c3)n2)cc1]

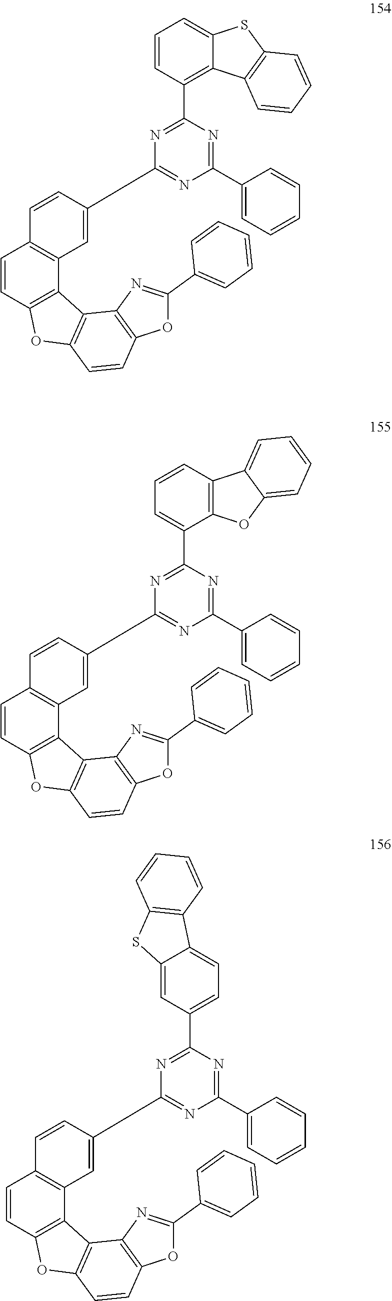 [SMILES: c1ccc(-c2nc(-c3ccc4ccc5oc6ccc7oc(-c8ccccc8)nc7c6c5c4c3)nc(-c3cccc4sc5ccccc5c34)n2)cc1 ; c1ccc(-c2nc(-c3ccc4ccc5oc6ccc7oc(-c8ccccc8)nc7c6c5c4c3)nc(-c3cccc4c3oc3ccccc34)n2)cc1 ; c1ccc(-c2nc(-c3ccc4c(c3)sc3ccccc34)nc(-c3ccc4ccc5oc6ccc7oc(-c8ccccc8)nc7c6c5c4c3)n2)cc1]

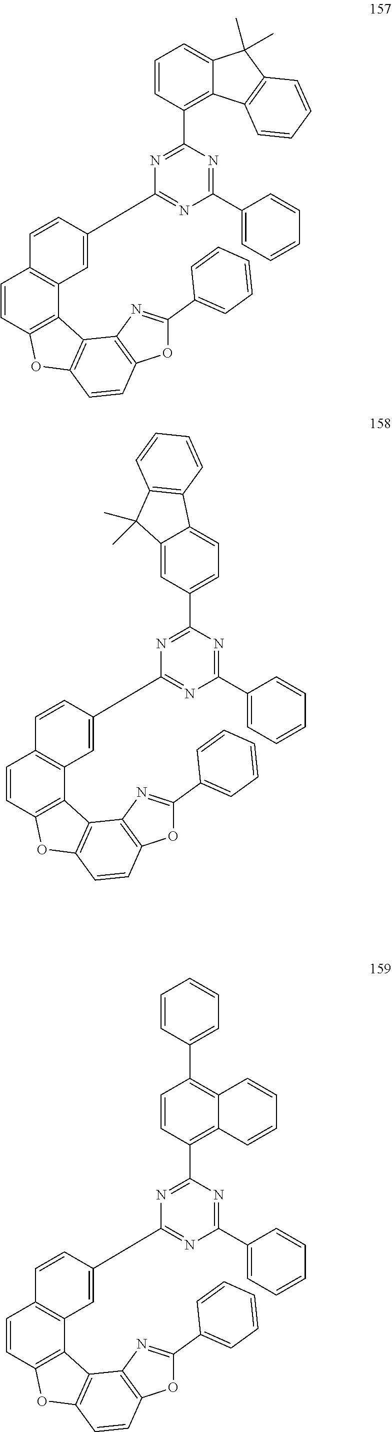 [SMILES: CC1(C)c2ccccc2-c2c(-c3nc(-c4ccccc4)nc(-c4ccc5ccc6oc7ccc8oc(-c9ccccc9)nc8c7c6c5c4)n3)cccc21 ; CC1(C)c2ccccc2-c2ccc(-c3nc(-c4ccccc4)nc(-c4ccc5ccc6oc7ccc8oc(-c9ccccc9)nc8c7c6c5c4)n3)cc21 ; c1ccc(-c2nc(-c3ccc4ccc5oc6ccc7oc(-c8ccccc8)nc7c6c5c4c3)nc(-c3ccc(-c4ccccc4)c4ccccc34)n2)cc1]

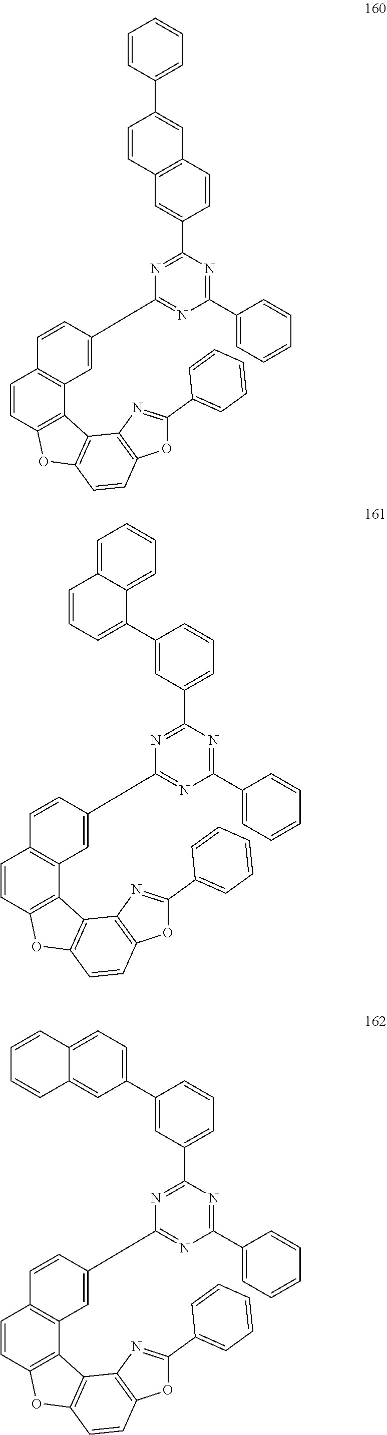

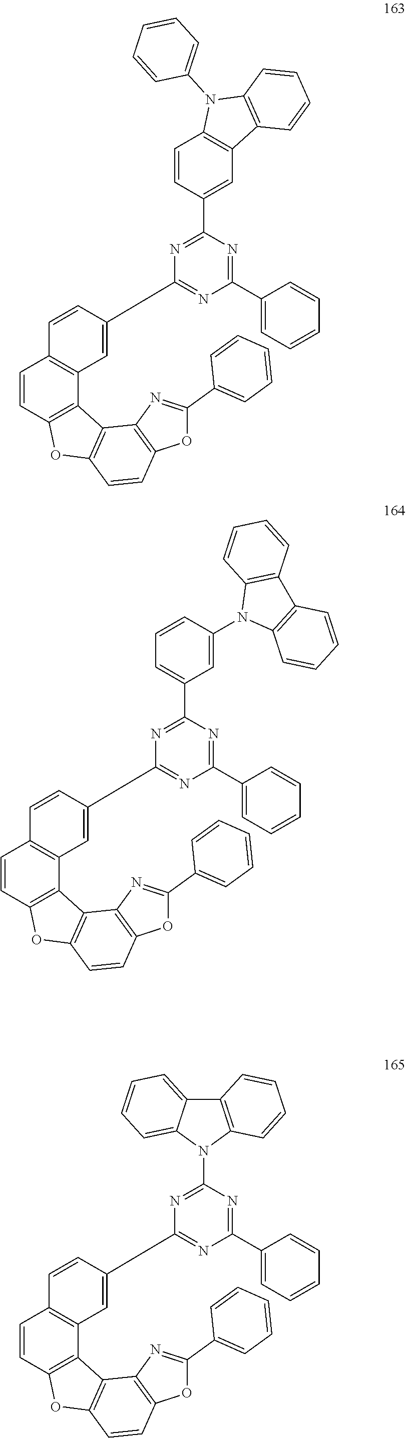 [SMILES: c1ccc(-c2nc(-c3ccc4ccc5oc6ccc7oc(-c8ccccc8)nc7c6c5c4c3)nc(-c3ccc4c(c3)c3ccccc3n4-c3ccccc3)n2)cc1 ; c1ccc(-c2nc(-c3cccc(-n4c5ccccc5c5ccccc54)c3)nc(-c3ccc4ccc5oc6ccc7oc(-c8ccccc8)nc7c6c5c4c3)n2)cc1 ; c1ccc(-c2nc(-c3ccc4ccc5oc6ccc7oc(-c8ccccc8)nc7c6c5c4c3)nc(-n3c4ccccc4c4ccccc43)n2)cc1]

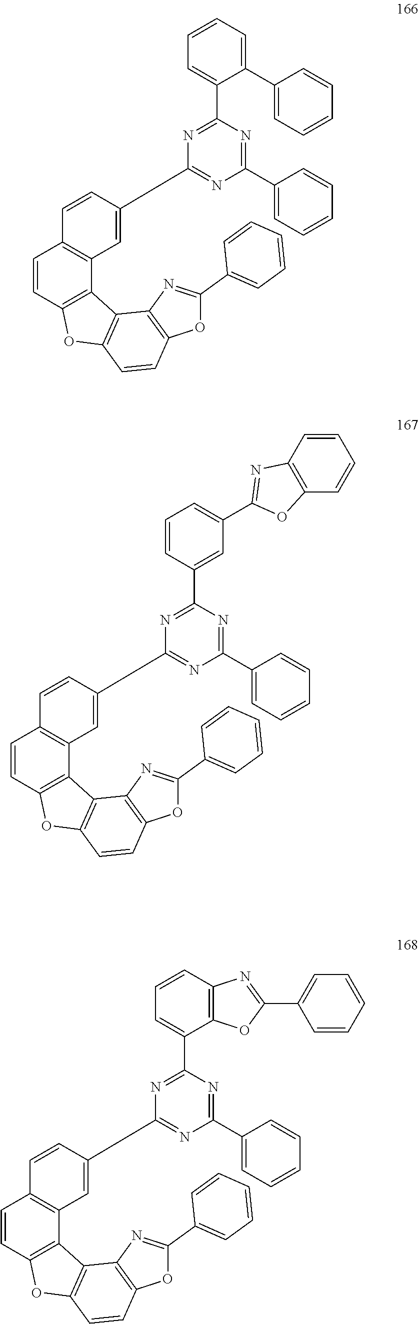 [SMILES: c1ccc(-c2nc(-c3ccc4ccc5oc6ccc7oc(-c8ccccc8)nc7c6c5c4c3)nc(-c3ccccc3-c3ccccc3)n2)cc1 ; c1ccc(-c2nc(-c3cccc(-c4nc5ccccc5o4)c3)nc(-c3ccc4ccc5oc6ccc7oc(-c8ccccc8)nc7c6c5c4c3)n2)cc1 ; c1ccc(-c2nc(-c3ccc4ccc5oc6ccc7oc(-c8ccccc8)nc7c6c5c4c3)nc(-c3cccc4nc(-c5ccccc5)oc34)n2)cc1]

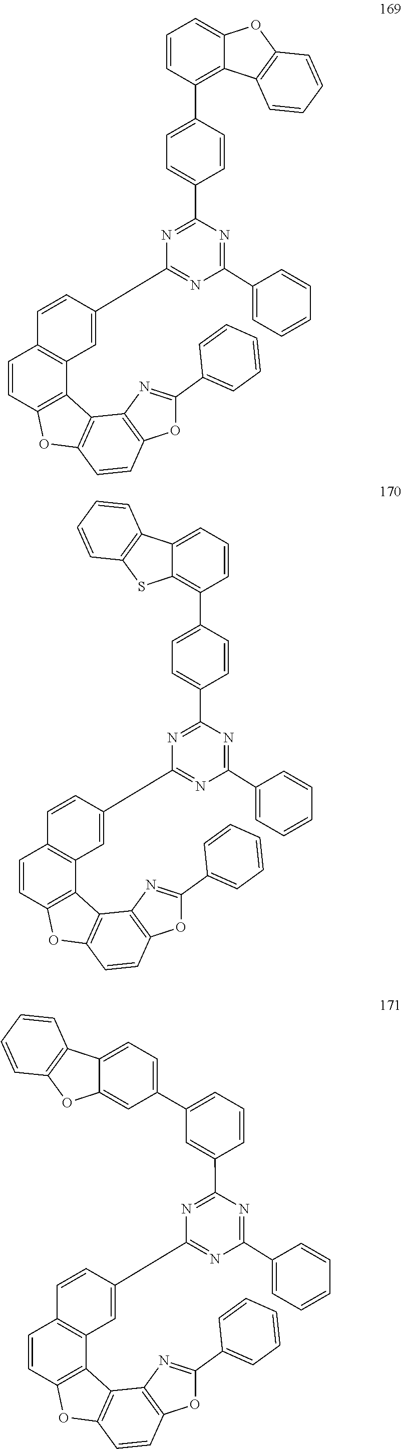

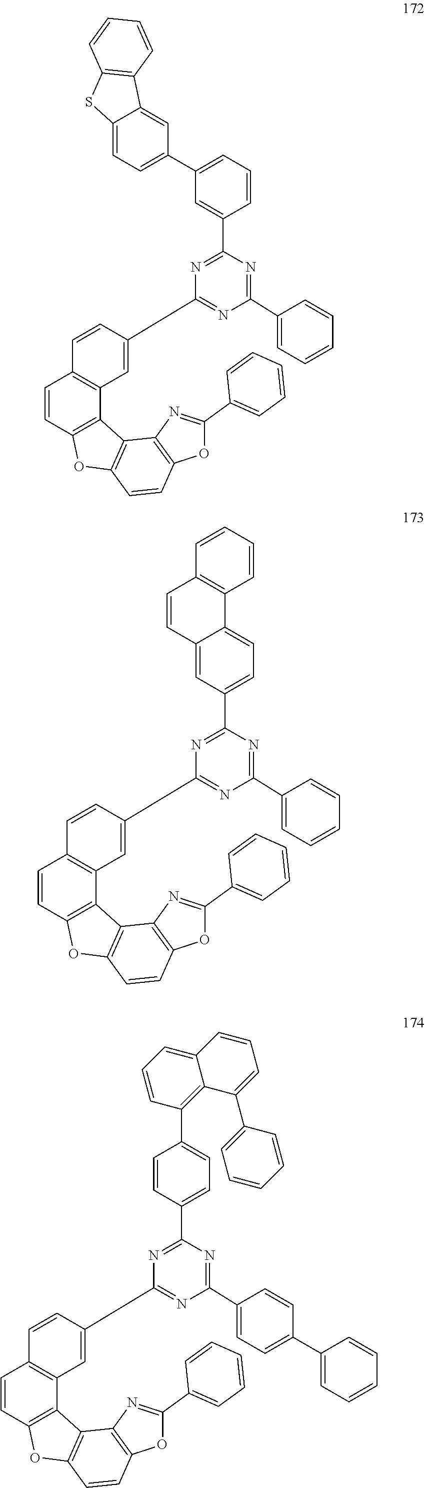 [SMILES: c1ccc(-c2nc(-c3cccc(-c4ccc5sc6ccccc6c5c4)c3)nc(-c3ccc4ccc5oc6ccc7oc(-c8ccccc8)nc7c6c5c4c3)n2)cc1 ; c1ccc(-c2nc(-c3ccc4c(ccc5ccccc54)c3)nc(-c3ccc4ccc5oc6ccc7oc(-c8ccccc8)nc7c6c5c4c3)n2)cc1 ; c1ccc(-c2ccc(-c3nc(-c4ccc(-c5cccc6cccc(-c7ccccc7)c56)cc4)nc(-c4ccc5ccc6oc7ccc8oc(-c9ccccc9)nc8c7c6c5c4)n3)cc2)cc1]

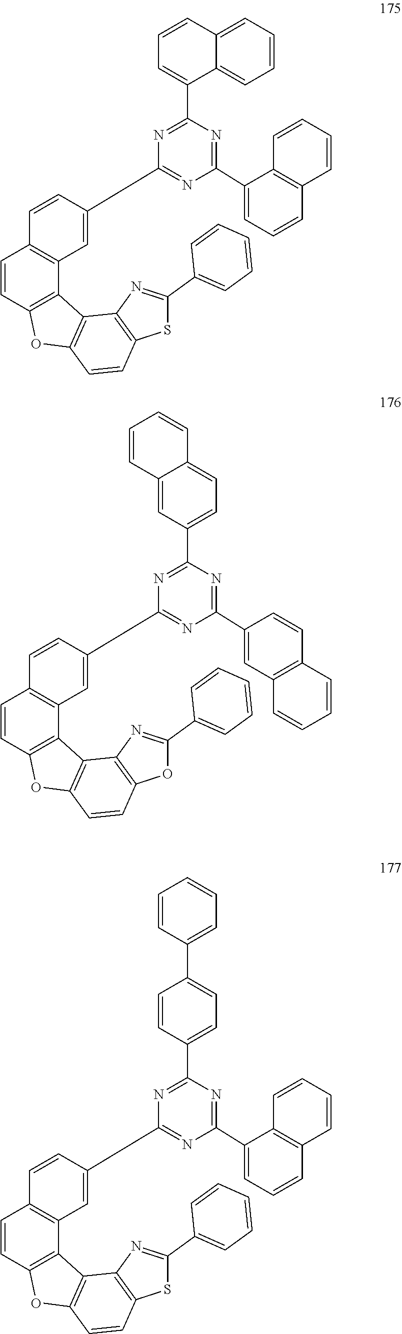 [SMILES: c1ccc(-c2nc3c(ccc4oc5ccc6ccc(-c7nc(-c8cccc9ccccc89)nc(-c8cccc9ccccc89)n7)cc6c5c43)s2)cc1 ; c1ccc(-c2nc3c(ccc4oc5ccc6ccc(-c7nc(-c8ccc9ccccc9c8)nc(-c8ccc9ccccc9c8)n7)cc6c5c43)o2)cc1 ; c1ccc(-c2ccc(-c3nc(-c4ccc5ccc6oc7ccc8sc(-c9ccccc9)nc8c7c6c5c4)nc(-c4cccc5ccccc45)n3)cc2)cc1]

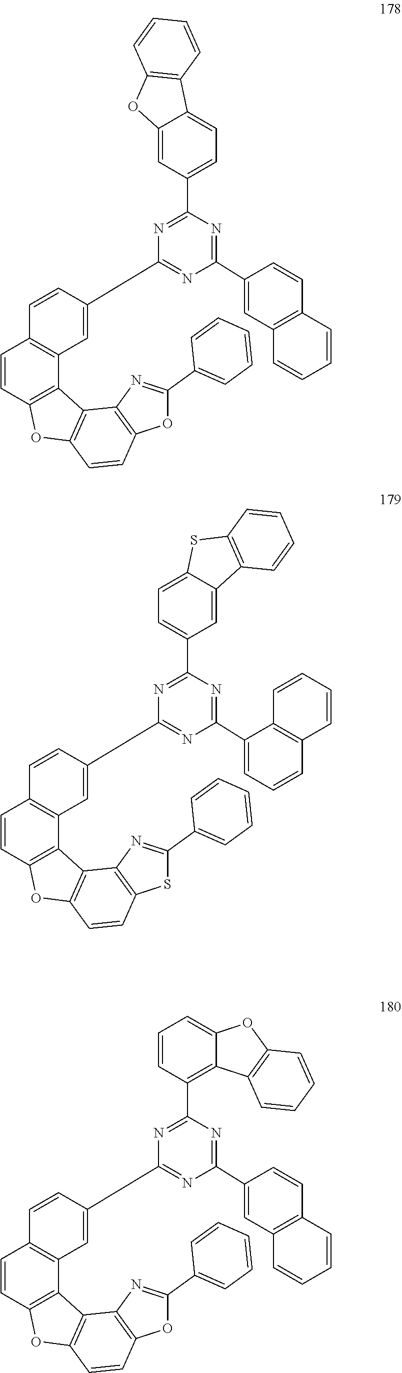 [SMILES: c1ccc(-c2nc3c(ccc4oc5ccc6ccc(-c7nc(-c8ccc9ccccc9c8)nc(-c8ccc9c(c8)oc8ccccc89)n7)cc6c5c43)o2)cc1 ; c1ccc(-c2nc3c(ccc4oc5ccc6ccc(-c7nc(-c8ccc9sc%10ccccc%10c9c8)nc(-c8cccc9ccccc89)n7)cc6c5c43)s2)cc1 ; c1ccc(-c2nc3c(ccc4oc5ccc6ccc(-c7nc(-c8ccc9ccccc9c8)nc(-c8cccc9oc%10ccccc%10c89)n7)cc6c5c43)o2)cc1]

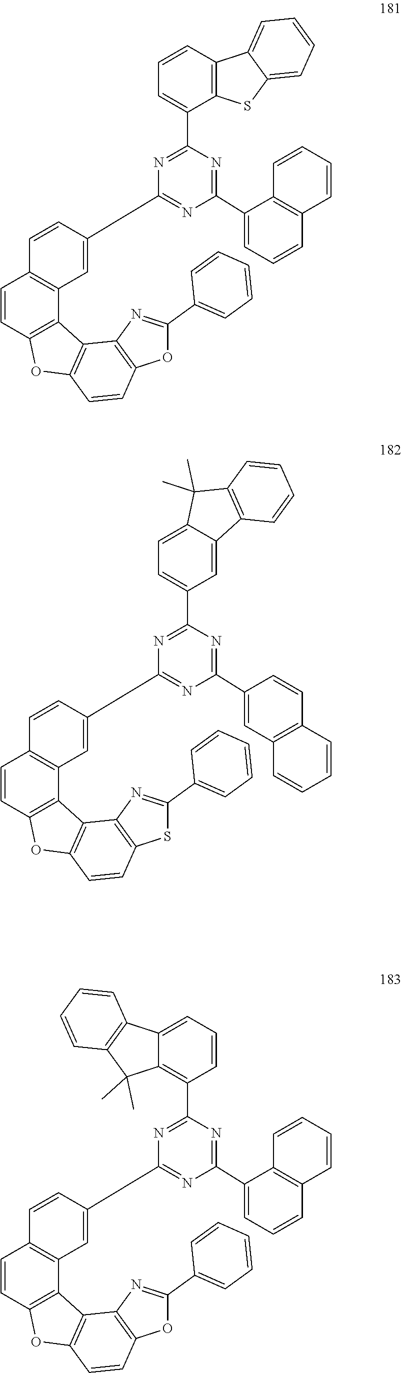 [SMILES: c1ccc(-c2nc3c(ccc4oc5ccc6ccc(-c7nc(-c8cccc9ccccc89)nc(-c8cccc9c8sc8ccccc89)n7)cc6c5c43)o2)cc1 ; CC1(C)c2ccccc2-c2cc(-c3nc(-c4ccc5ccccc5c4)nc(-c4ccc5ccc6oc7ccc8sc(-c9ccccc9)nc8c7c6c5c4)n3)ccc21 ; CC1(C)c2ccccc2-c2cccc(-c3nc(-c4ccc5ccc6oc7ccc8oc(-c9ccccc9)nc8c7c6c5c4)nc(-c4cccc5ccccc45)n3)c21]

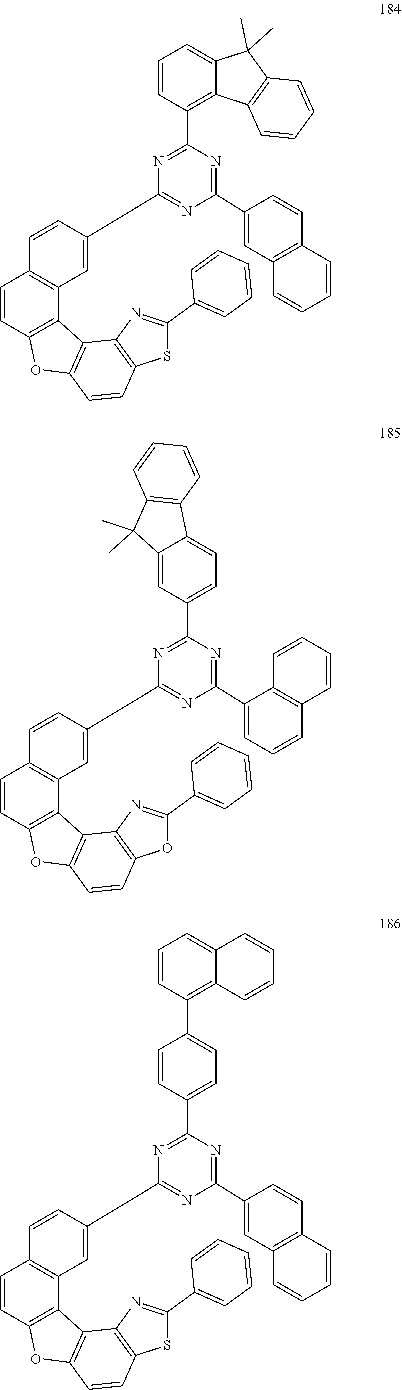 [SMILES: CC1(C)c2ccccc2-c2c(-c3nc(-c4ccc5ccccc5c4)nc(-c4ccc5ccc6oc7ccc8sc(-c9ccccc9)nc8c7c6c5c4)n3)cccc21 ; CC1(C)c2ccccc2-c2ccc(-c3nc(-c4ccc5ccc6oc7ccc8oc(-c9ccccc9)nc8c7c6c5c4)nc(-c4cccc5ccccc45)n3)cc21 ; c1ccc(-c2nc3c(ccc4oc5ccc6ccc(-c7nc(-c8ccc(-c9cccc%10ccccc9%10)cc8)nc(-c8ccc9ccccc9c8)n7)cc6c5c43)s2)cc1]

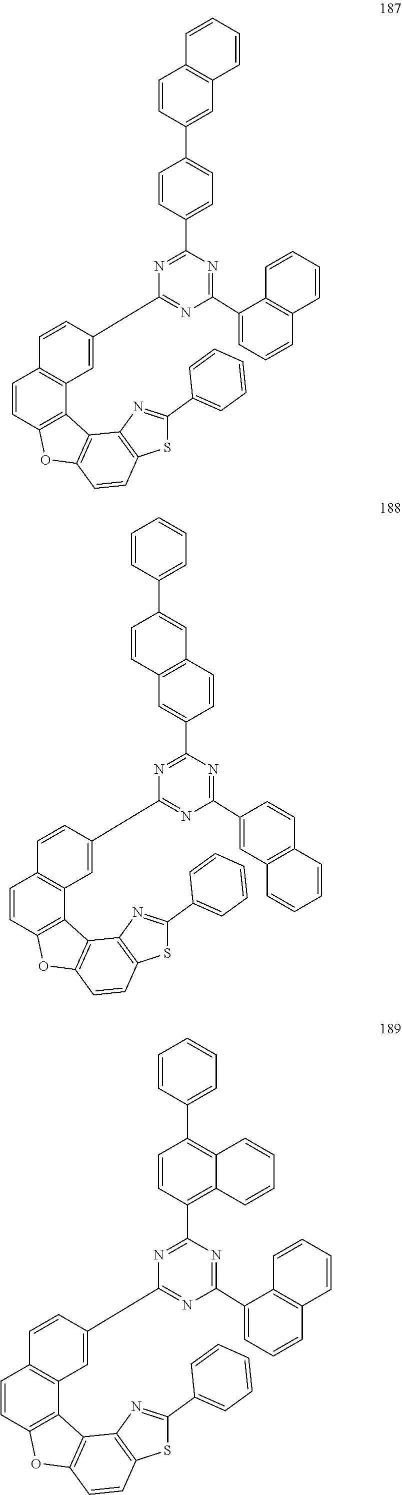 [SMILES: c1ccc(-c2nc3c(ccc4oc5ccc6ccc(-c7nc(-c8ccc(-c9ccc%10ccccc%10c9)cc8)nc(-c8cccc9ccccc89)n7)cc6c5c43)s2)cc1 ; c1ccc(-c2ccc3cc(-c4nc(-c5ccc6ccccc6c5)nc(-c5ccc6ccc7oc8ccc9sc(-c%10ccccc%10)nc9c8c7c6c5)n4)ccc3c2)cc1 ; c1ccc(-c2nc3c(ccc4oc5ccc6ccc(-c7nc(-c8cccc9ccccc89)nc(-c8ccc(-c9ccccc9)c9ccccc89)n7)cc6c5c43)s2)cc1]

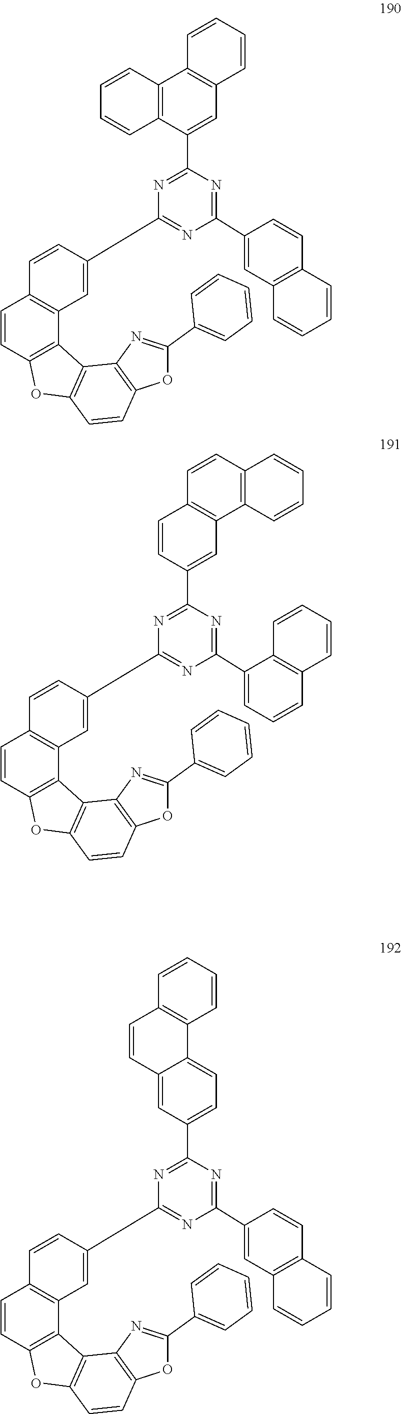 [SMILES: c1ccc(-c2nc3c(ccc4oc5ccc6ccc(-c7nc(-c8ccc9ccccc9c8)nc(-c8cc9ccccc9c9ccccc89)n7)cc6c5c43)o2)cc1 ; c1ccc(-c2nc3c(ccc4oc5ccc6ccc(-c7nc(-c8ccc9ccc%10ccccc%10c9c8)nc(-c8cccc9ccccc89)n7)cc6c5c43)o2)cc1 ; c1ccc(-c2nc3c(ccc4oc5ccc6ccc(-c7nc(-c8ccc9ccccc9c8)nc(-c8ccc9c(ccc%10ccccc%109)c8)n7)cc6c5c43)o2)cc1]

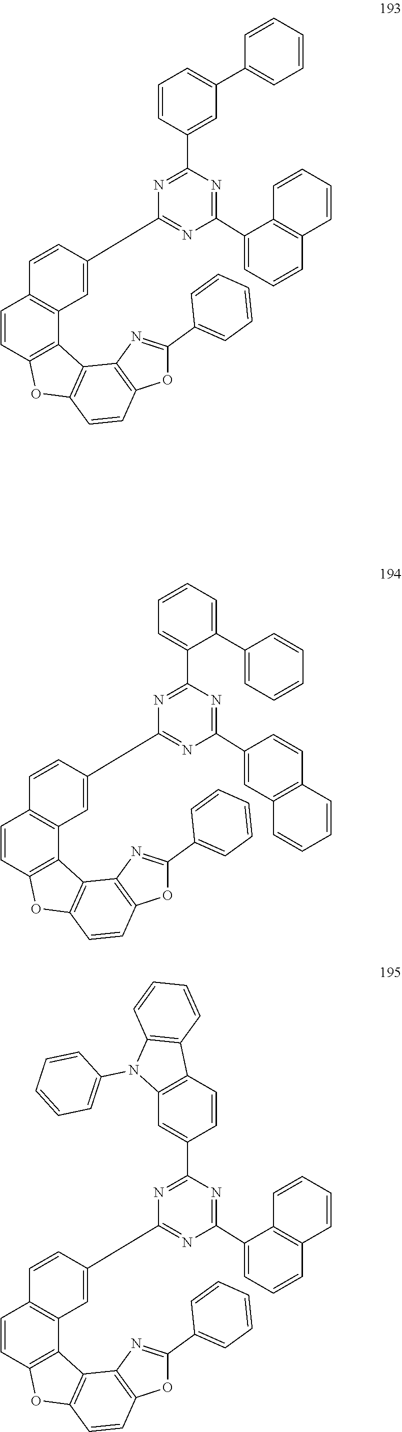 [SMILES: c1ccc(-c2cccc(-c3nc(-c4ccc5ccc6oc7ccc8oc(-c9ccccc9)nc8c7c6c5c4)nc(-c4cccc5ccccc45)n3)c2)cc1 ; c1ccc(-c2nc3c(ccc4oc5ccc6ccc(-c7nc(-c8ccc9ccccc9c8)nc(-c8ccccc8-c8ccccc8)n7)cc6c5c43)o2)cc1 ; c1ccc(-c2nc3c(ccc4oc5ccc6ccc(-c7nc(-c8ccc9c%10ccccc%10n(-c%10ccccc%10)c9c8)nc(-c8cccc9ccccc89)n7)cc6c5c43)o2)cc1]

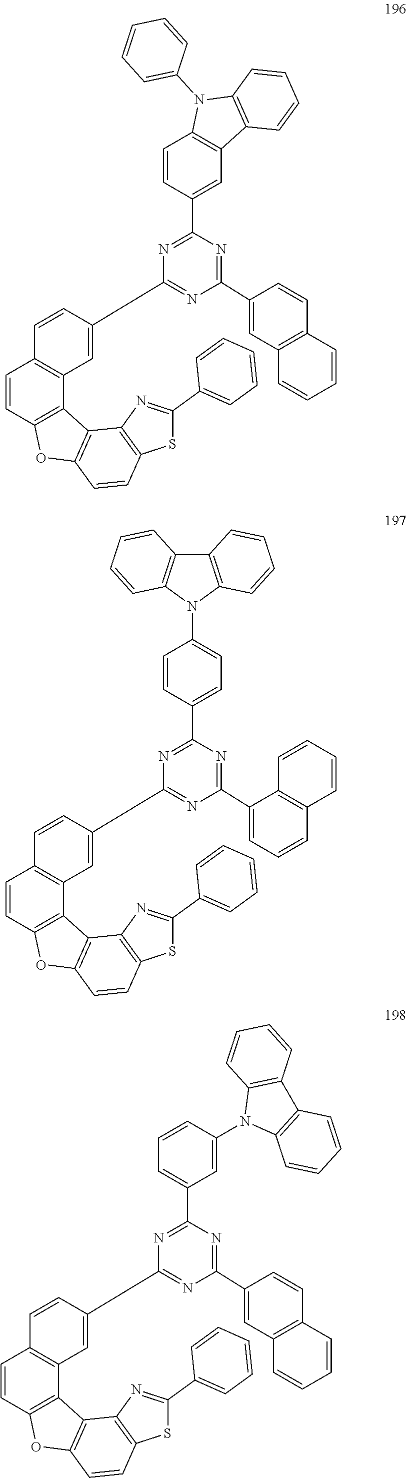 [SMILES: c1ccc(-c2nc3c(ccc4oc5ccc6ccc(-c7nc(-c8ccc9ccccc9c8)nc(-c8ccc9c(c8)c8ccccc8n9-c8ccccc8)n7)cc6c5c43)s2)cc1 ; c1ccc(-c2nc3c(ccc4oc5ccc6ccc(-c7nc(-c8ccc(-n9c%10ccccc%10c%10ccccc%109)cc8)nc(-c8cccc9ccccc89)n7)cc6c5c43)s2)cc1 ; c1ccc(-c2nc3c(ccc4oc5ccc6ccc(-c7nc(-c8cccc(-n9c%10ccccc%10c%10ccccc%109)c8)nc(-c8ccc9ccccc9c8)n7)cc6c5c43)s2)cc1]

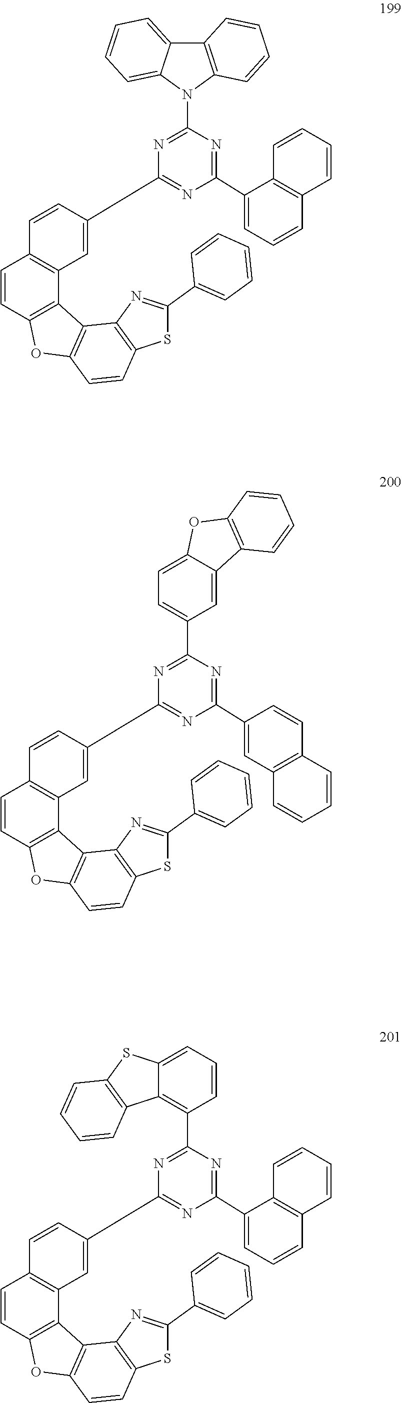 [SMILES: c1ccc(-c2nc3c(ccc4oc5ccc6ccc(-c7nc(-c8cccc9ccccc89)nc(-n8c9ccccc9c9ccccc98)n7)cc6c5c43)s2)cc1 ; c1ccc(-c2nc3c(ccc4oc5ccc6ccc(-c7nc(-c8ccc9ccccc9c8)nc(-c8ccc9oc%10ccccc%10c9c8)n7)cc6c5c43)s2)cc1 ; c1ccc(-c2nc3c(ccc4oc5ccc6ccc(-c7nc(-c8cccc9ccccc89)nc(-c8cccc9sc%10ccccc%10c89)n7)cc6c5c43)s2)cc1]

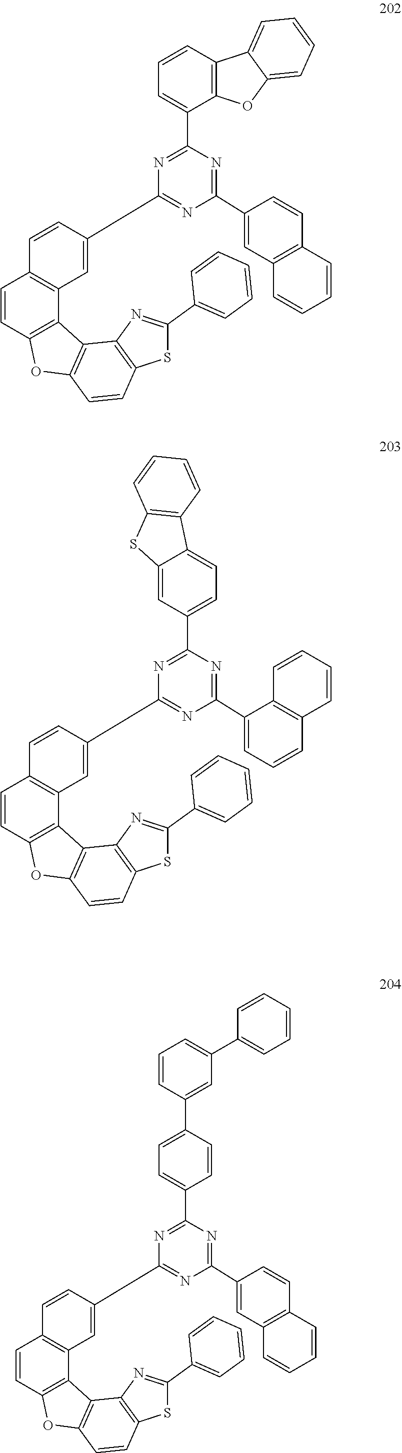 [SMILES: c1ccc(-c2nc3c(ccc4oc5ccc6ccc(-c7nc(-c8ccc9ccccc9c8)nc(-c8cccc9c8oc8ccccc89)n7)cc6c5c43)s2)cc1 ; c1ccc(-c2nc3c(ccc4oc5ccc6ccc(-c7nc(-c8ccc9c(c8)sc8ccccc89)nc(-c8cccc9ccccc89)n7)cc6c5c43)s2)cc1 ; c1ccc(-c2cccc(-c3ccc(-c4nc(-c5ccc6ccccc6c5)nc(-c5ccc6ccc7oc8ccc9sc(-c%10ccccc%10)nc9c8c7c6c5)n4)cc3)c2)cc1]

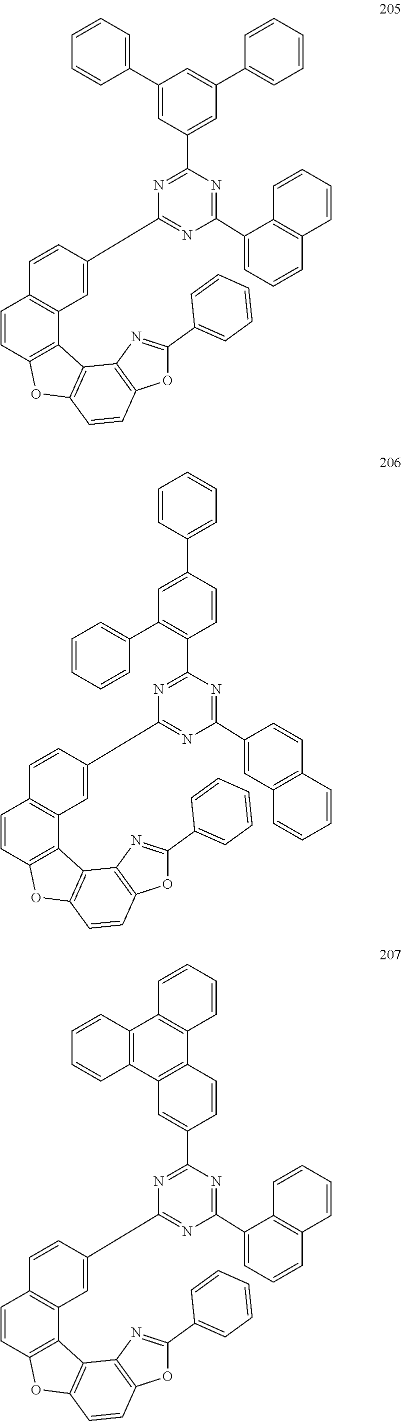 [SMILES: c1ccc(-c2cc(-c3ccccc3)cc(-c3nc(-c4ccc5ccc6oc7ccc8oc(-c9ccccc9)nc8c7c6c5c4)nc(-c4cccc5ccccc45)n3)c2)cc1 ; c1ccc(-c2ccc(-c3nc(-c4ccc5ccccc5c4)nc(-c4ccc5ccc6oc7ccc8oc(-c9ccccc9)nc8c7c6c5c4)n3)c(-c3ccccc3)c2)cc1 ; c1ccc(-c2nc3c(ccc4oc5ccc6ccc(-c7nc(-c8ccc9c%10ccccc%10c%10ccccc%10c9c8)nc(-c8cccc9ccccc89)n7)cc6c5c43)o2)cc1]

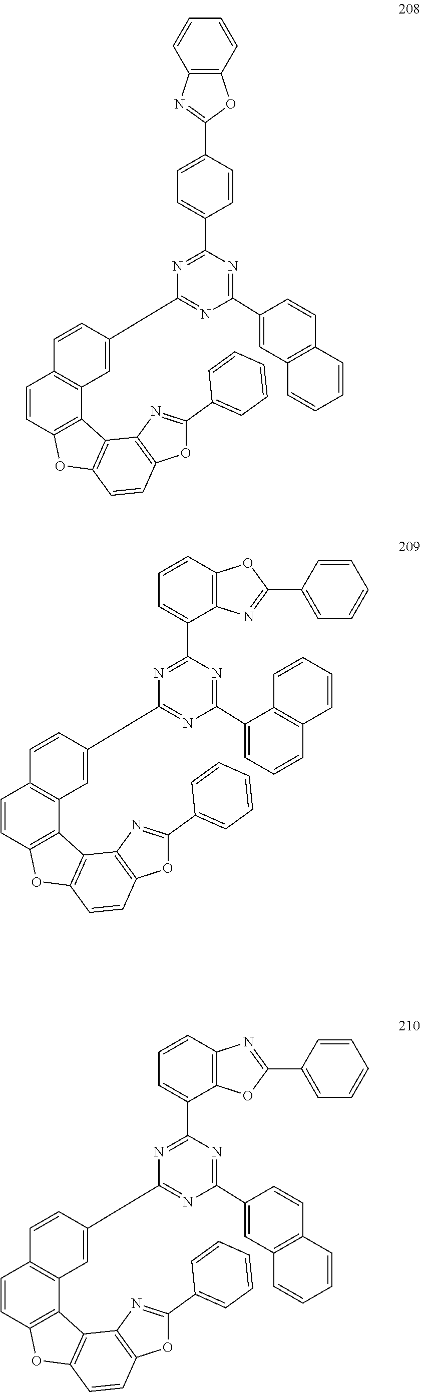 [SMILES: c1ccc(-c2nc3c(ccc4oc5ccc6ccc(-c7nc(-c8ccc(-c9nc%10ccccc%10o9)cc8)nc(-c8ccc9ccccc9c8)n7)cc6c5c43)o2)cc1 ; c1ccc(-c2nc3c(-c4nc(-c5ccc6ccc7oc8ccc9oc(-c%10ccccc%10)nc9c8c7c6c5)nc(-c5cccc6ccccc56)n4)cccc3o2)cc1 ; c1ccc(-c2nc3c(ccc4oc5ccc6ccc(-c7nc(-c8ccc9ccccc9c8)nc(-c8cccc9nc(-c%10ccccc%10)oc89)n7)cc6c5c43)o2)cc1]

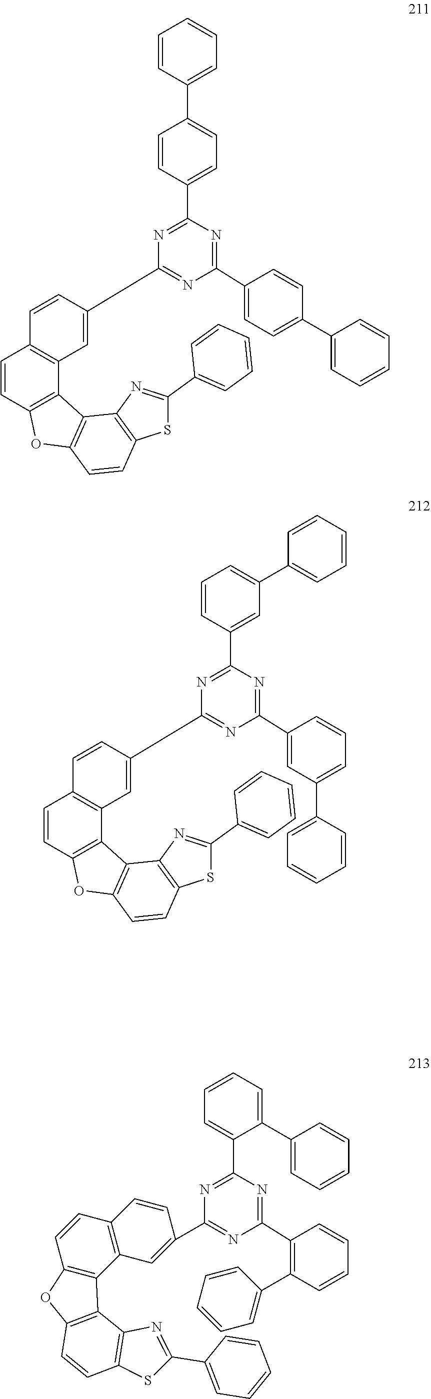 [SMILES: c1ccc(-c2ccc(-c3nc(-c4ccc(-c5ccccc5)cc4)nc(-c4ccc5ccc6oc7ccc8sc(-c9ccccc9)nc8c7c6c5c4)n3)cc2)cc1 ; c1ccc(-c2cccc(-c3nc(-c4cccc(-c5ccccc5)c4)nc(-c4ccc5ccc6oc7ccc8sc(-c9ccccc9)nc8c7c6c5c4)n3)c2)cc1 ; c1ccc(-c2nc3c(ccc4oc5ccc6ccc(-c7nc(-c8ccccc8-c8ccccc8)nc(-c8ccccc8-c8ccccc8)n7)cc6c5c43)s2)cc1]

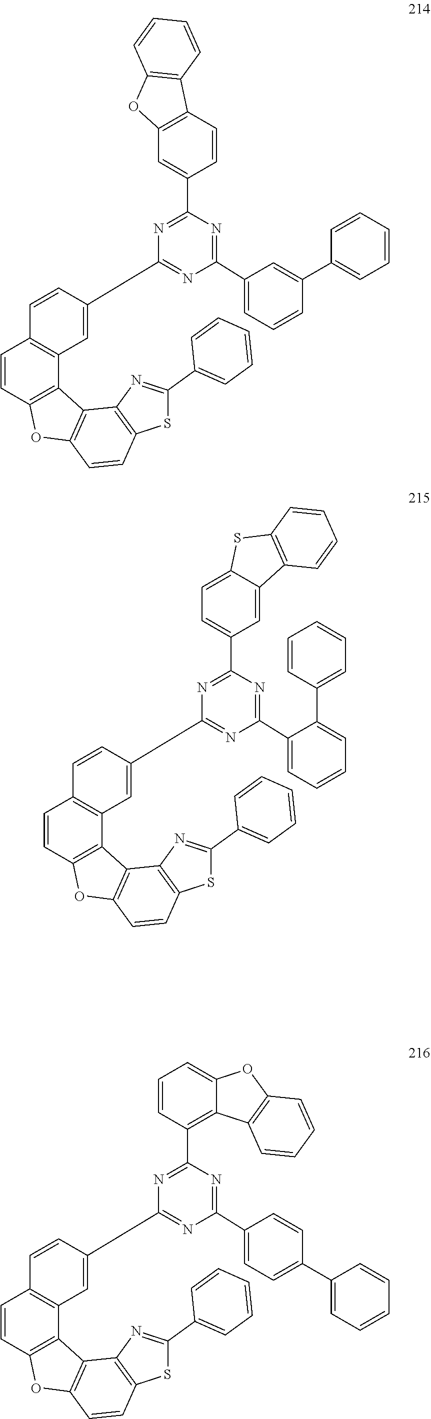 [SMILES: c1ccc(-c2cccc(-c3nc(-c4ccc5c(c4)oc4ccccc45)nc(-c4ccc5ccc6oc7ccc8sc(-c9ccccc9)nc8c7c6c5c4)n3)c2)cc1 ; c1ccc(-c2nc3c(ccc4oc5ccc6ccc(-c7nc(-c8ccc9sc%10ccccc%10c9c8)nc(-c8ccccc8-c8ccccc8)n7)cc6c5c43)s2)cc1 ; c1ccc(-c2ccc(-c3nc(-c4ccc5ccc6oc7ccc8sc(-c9ccccc9)nc8c7c6c5c4)nc(-c4cccc5oc6ccccc6c45)n3)cc2)cc1]

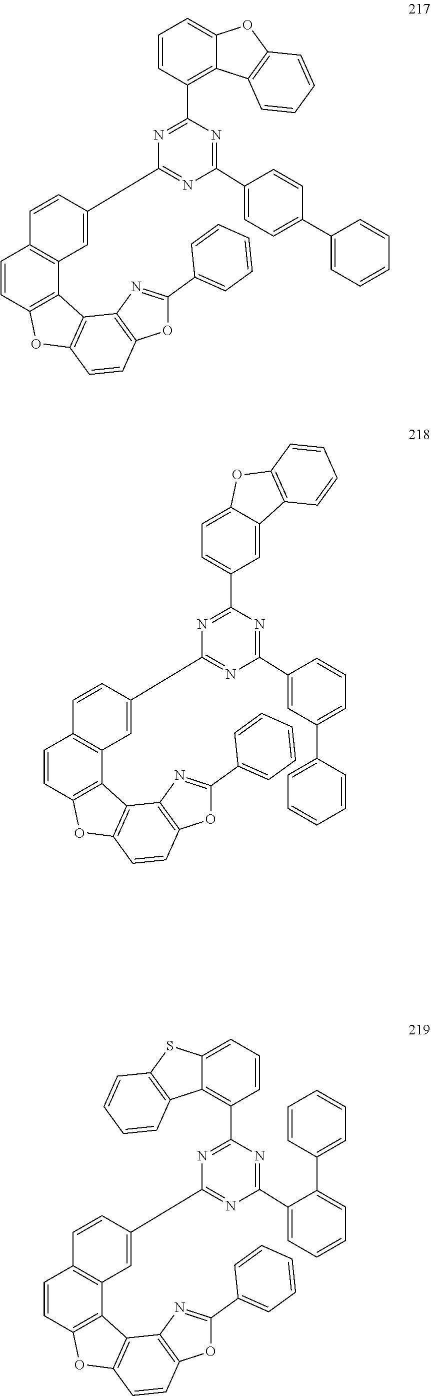 [SMILES: c1ccc(-c2ccc(-c3nc(-c4ccc5ccc6oc7ccc8oc(-c9ccccc9)nc8c7c6c5c4)nc(-c4cccc5oc6ccccc6c45)n3)cc2)cc1 ; c1ccc(-c2cccc(-c3nc(-c4ccc5oc6ccccc6c5c4)nc(-c4ccc5ccc6oc7ccc8oc(-c9ccccc9)nc8c7c6c5c4)n3)c2)cc1 ; c1ccc(-c2nc3c(ccc4oc5ccc6ccc(-c7nc(-c8ccccc8-c8ccccc8)nc(-c8cccc9sc%10ccccc%10c89)n7)cc6c5c43)o2)cc1]

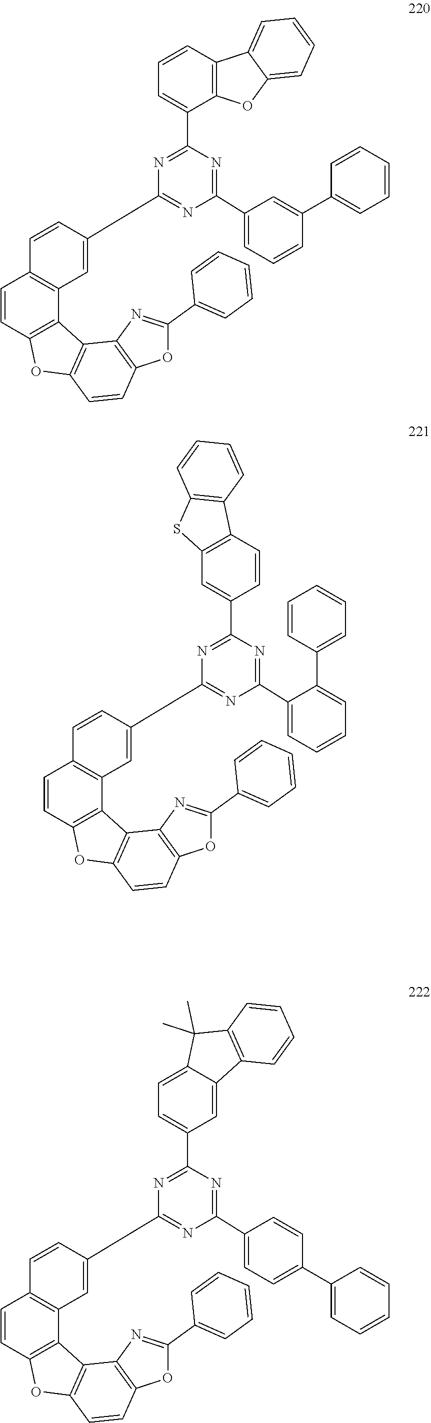 [SMILES: c1ccc(-c2cccc(-c3nc(-c4ccc5ccc6oc7ccc8oc(-c9ccccc9)nc8c7c6c5c4)nc(-c4cccc5c4oc4ccccc45)n3)c2)cc1 ; c1ccc(-c2nc3c(ccc4oc5ccc6ccc(-c7nc(-c8ccc9c(c8)sc8ccccc89)nc(-c8ccccc8-c8ccccc8)n7)cc6c5c43)o2)cc1 ; CC1(C)c2ccccc2-c2cc(-c3nc(-c4ccc(-c5ccccc5)cc4)nc(-c4ccc5ccc6oc7ccc8oc(-c9ccccc9)nc8c7c6c5c4)n3)ccc21]

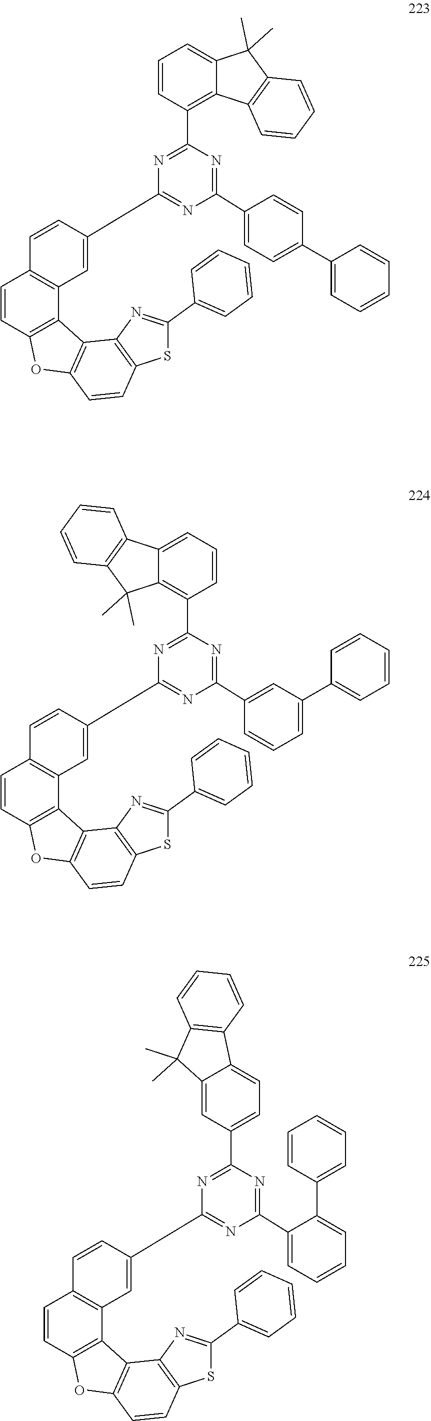 [SMILES: CC1(C)c2ccccc2-c2c(-c3nc(-c4ccc(-c5ccccc5)cc4)nc(-c4ccc5ccc6oc7ccc8sc(-c9ccccc9)nc8c7c6c5c4)n3)cccc21 ; CC1(C)c2ccccc2-c2cccc(-c3nc(-c4cccc(-c5ccccc5)c4)nc(-c4ccc5ccc6oc7ccc8sc(-c9ccccc9)nc8c7c6c5c4)n3)c21 ; CC1(C)c2ccccc2-c2ccc(-c3nc(-c4ccc5ccc6oc7ccc8sc(-c9ccccc9)nc8c7c6c5c4)nc(-c4ccccc4-c4ccccc4)n3)cc21]

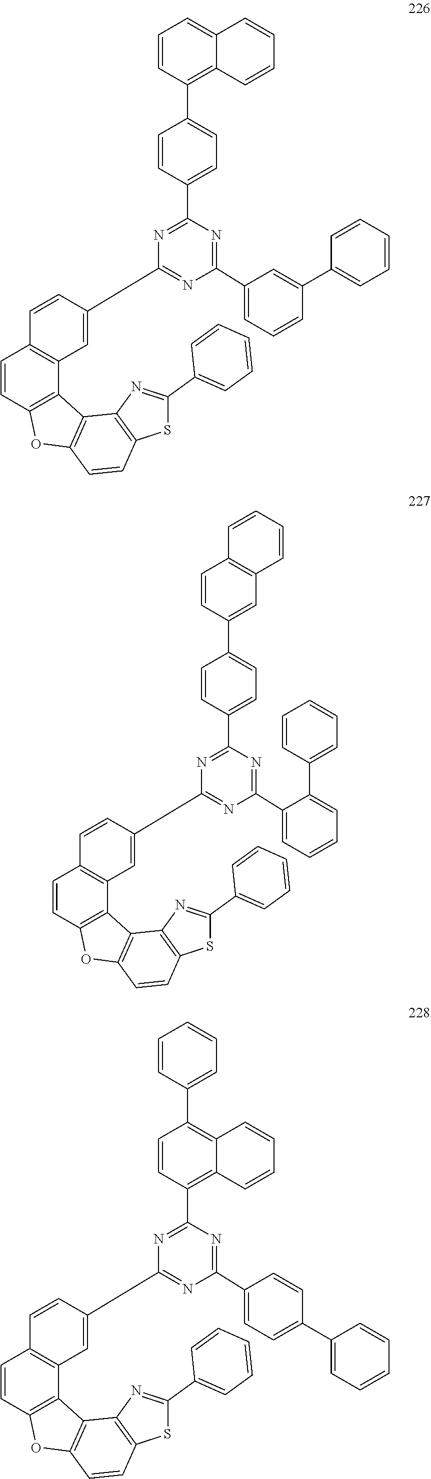

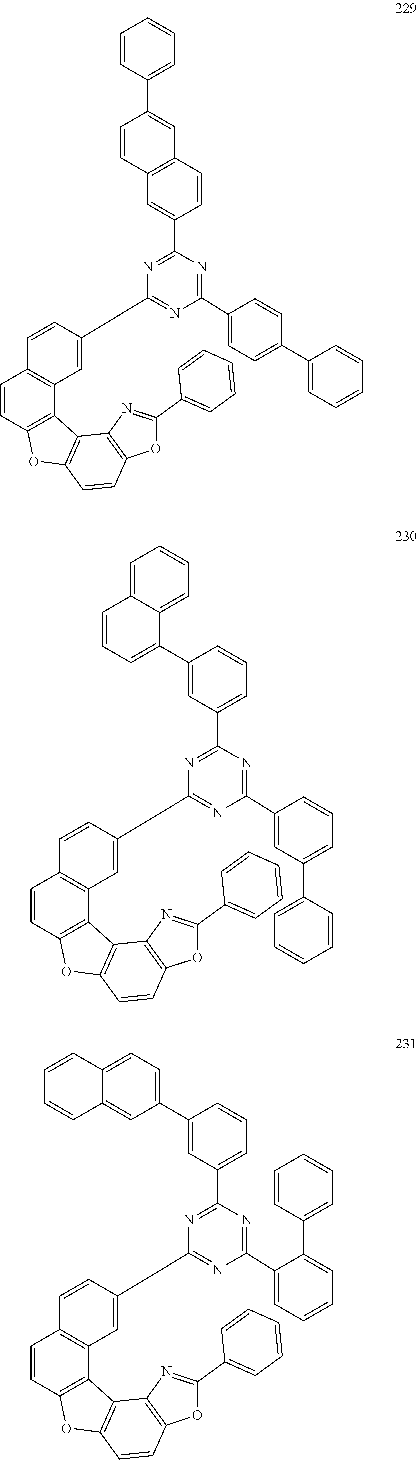 [SMILES: c1ccc(-c2ccc(-c3nc(-c4ccc5cc(-c6ccccc6)ccc5c4)nc(-c4ccc5ccc6oc7ccc8oc(-c9ccccc9)nc8c7c6c5c4)n3)cc2)cc1 ; c1ccc(-c2cccc(-c3nc(-c4cccc(-c5cccc6ccccc56)c4)nc(-c4ccc5ccc6oc7ccc8oc(-c9ccccc9)nc8c7c6c5c4)n3)c2)cc1 ; c1ccc(-c2nc3c(ccc4oc5ccc6ccc(-c7nc(-c8cccc(-c9ccc%10ccccc%10c9)c8)nc(-c8ccccc8-c8ccccc8)n7)cc6c5c43)o2)cc1]

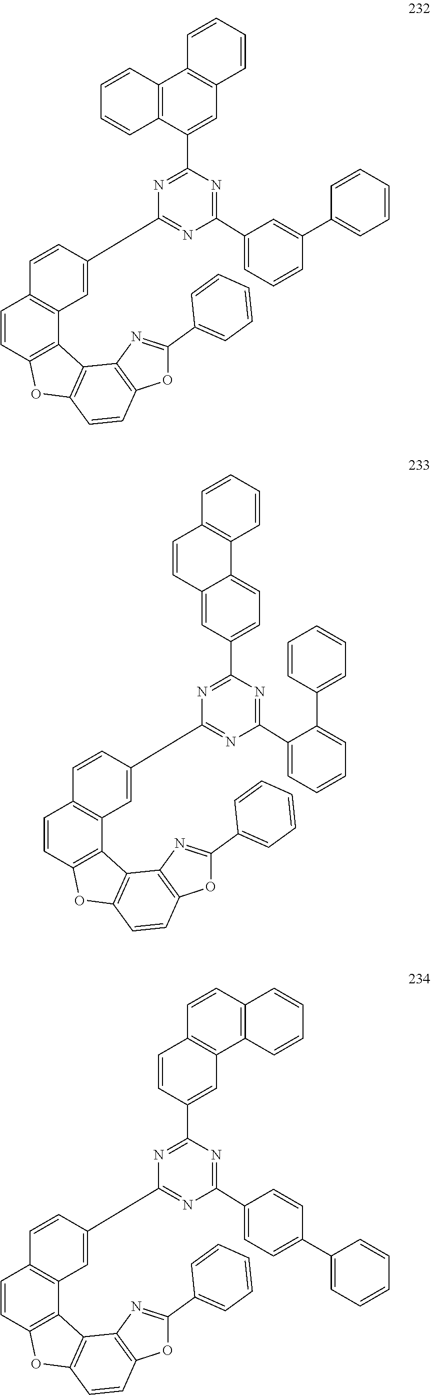 [SMILES: c1ccc(-c2cccc(-c3nc(-c4ccc5ccc6oc7ccc8oc(-c9ccccc9)nc8c7c6c5c4)nc(-c4cc5ccccc5c5ccccc45)n3)c2)cc1 ; c1ccc(-c2nc3c(ccc4oc5ccc6ccc(-c7nc(-c8ccc9c(ccc%10ccccc%109)c8)nc(-c8ccccc8-c8ccccc8)n7)cc6c5c43)o2)cc1 ; c1ccc(-c2ccc(-c3nc(-c4ccc5ccc6ccccc6c5c4)nc(-c4ccc5ccc6oc7ccc8oc(-c9ccccc9)nc8c7c6c5c4)n3)cc2)cc1]

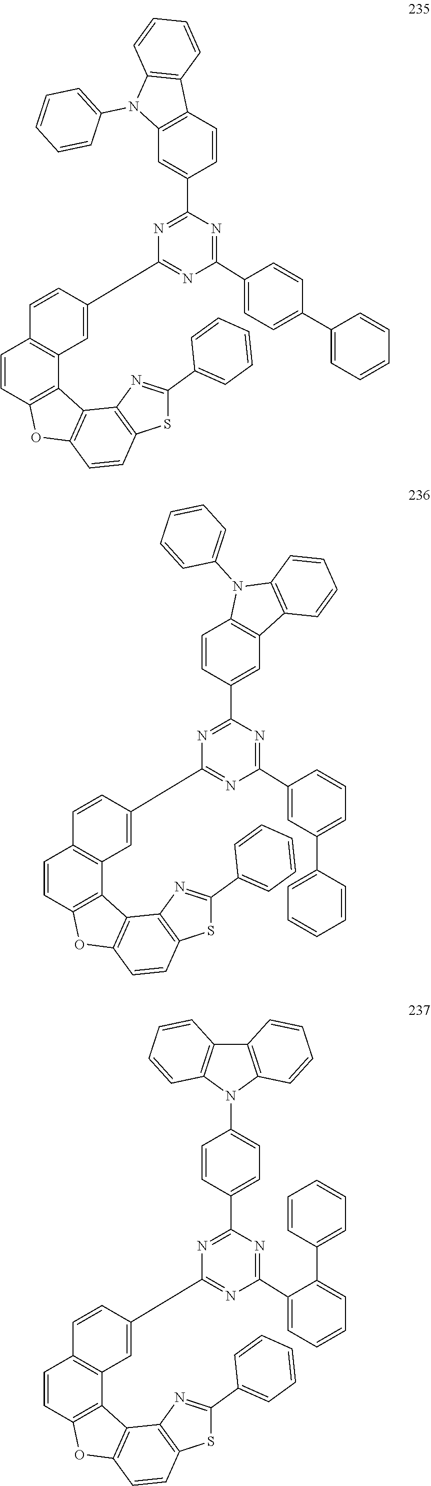 [SMILES: c1ccc(-c2ccc(-c3nc(-c4ccc5ccc6oc7ccc8sc(-c9ccccc9)nc8c7c6c5c4)nc(-c4ccc5c6ccccc6n(-c6ccccc6)c5c4)n3)cc2)cc1 ; c1ccc(-c2cccc(-c3nc(-c4ccc5ccc6oc7ccc8sc(-c9ccccc9)nc8c7c6c5c4)nc(-c4ccc5c(c4)c4ccccc4n5-c4ccccc4)n3)c2)cc1 ; c1ccc(-c2nc3c(ccc4oc5ccc6ccc(-c7nc(-c8ccc(-n9c%10ccccc%10c%10ccccc%109)cc8)nc(-c8ccccc8-c8ccccc8)n7)cc6c5c43)s2)cc1]

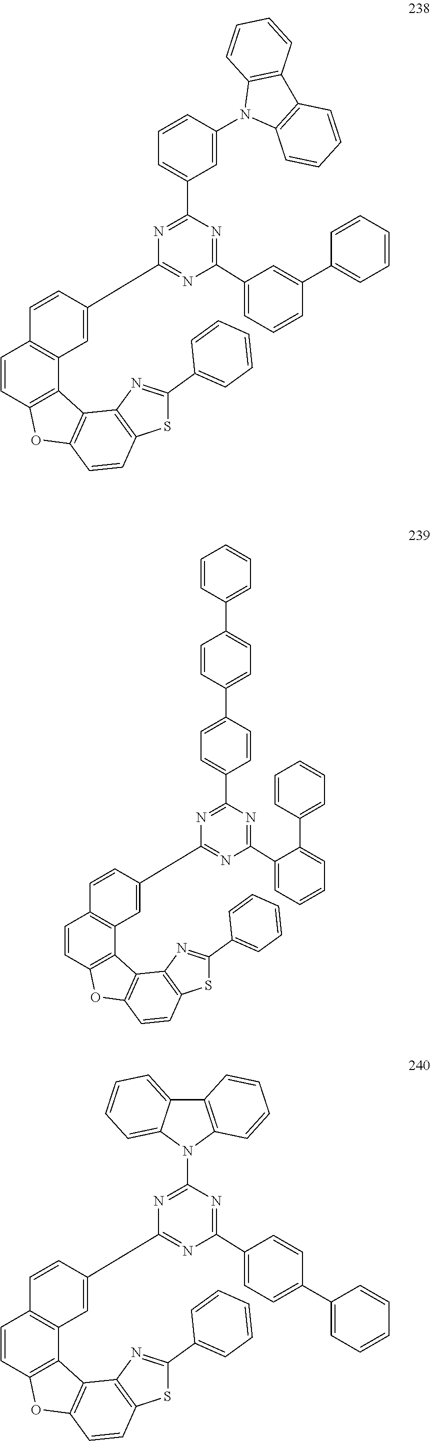 [SMILES: c1ccc(-c2cccc(-c3nc(-c4cccc(-n5c6ccccc6c6ccccc65)c4)nc(-c4ccc5ccc6oc7ccc8sc(-c9ccccc9)nc8c7c6c5c4)n3)c2)cc1 ; c1ccc(-c2ccc(-c3ccc(-c4nc(-c5ccc6ccc7oc8ccc9sc(-c%10ccccc%10)nc9c8c7c6c5)nc(-c5ccccc5-c5ccccc5)n4)cc3)cc2)cc1 ; c1ccc(-c2ccc(-c3nc(-c4ccc5ccc6oc7ccc8sc(-c9ccccc9)nc8c7c6c5c4)nc(-n4c5ccccc5c5ccccc54)n3)cc2)cc1]

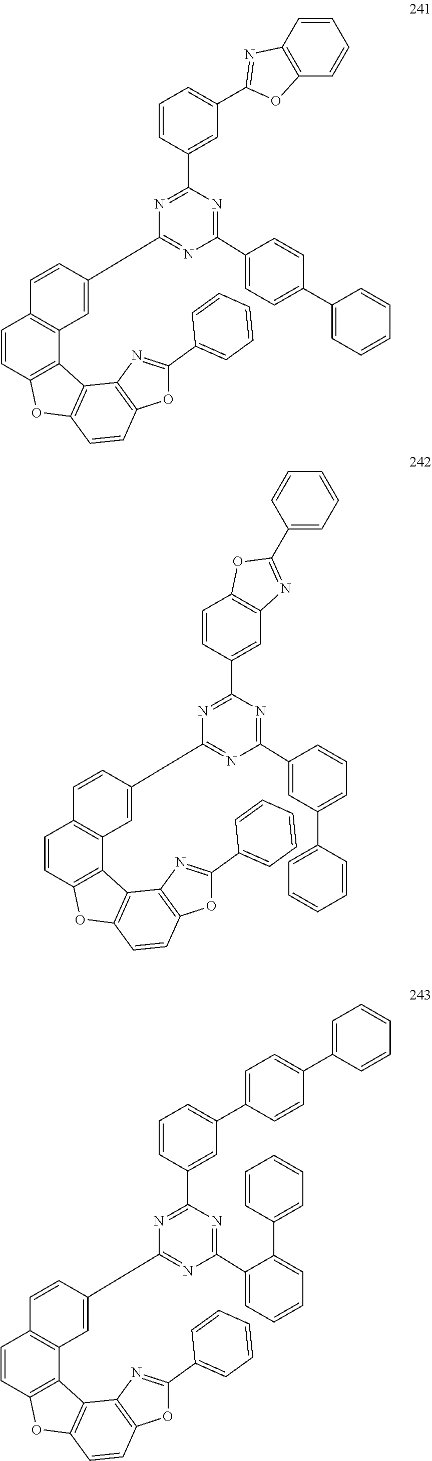 [SMILES: c1ccc(-c2ccc(-c3nc(-c4cccc(-c5nc6ccccc6o5)c4)nc(-c4ccc5ccc6oc7ccc8oc(-c9ccccc9)nc8c7c6c5c4)n3)cc2)cc1 ; c1ccc(-c2cccc(-c3nc(-c4ccc5oc(-c6ccccc6)nc5c4)nc(-c4ccc5ccc6oc7ccc8oc(-c9ccccc9)nc8c7c6c5c4)n3)c2)cc1 ; c1ccc(-c2ccc(-c3cccc(-c4nc(-c5ccc6ccc7oc8ccc9oc(-c%10ccccc%10)nc9c8c7c6c5)nc(-c5ccccc5-c5ccccc5)n4)c3)cc2)cc1]

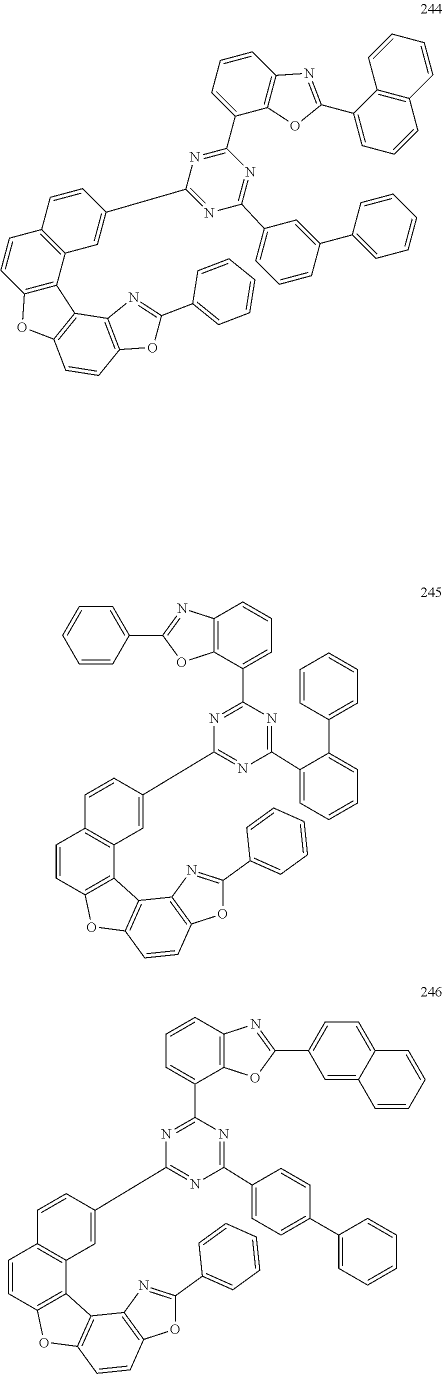 [SMILES: c1ccc(-c2cccc(-c3nc(-c4ccc5ccc6oc7ccc8oc(-c9ccccc9)nc8c7c6c5c4)nc(-c4cccc5nc(-c6cccc7ccccc67)oc45)n3)c2)cc1 ; c1ccc(-c2nc3c(ccc4oc5ccc6ccc(-c7nc(-c8ccccc8-c8ccccc8)nc(-c8cccc9nc(-c%10ccccc%10)oc89)n7)cc6c5c43)o2)cc1 ; c1ccc(-c2ccc(-c3nc(-c4ccc5ccc6oc7ccc8oc(-c9ccccc9)nc8c7c6c5c4)nc(-c4cccc5nc(-c6ccc7ccccc7c6)oc45)n3)cc2)cc1]

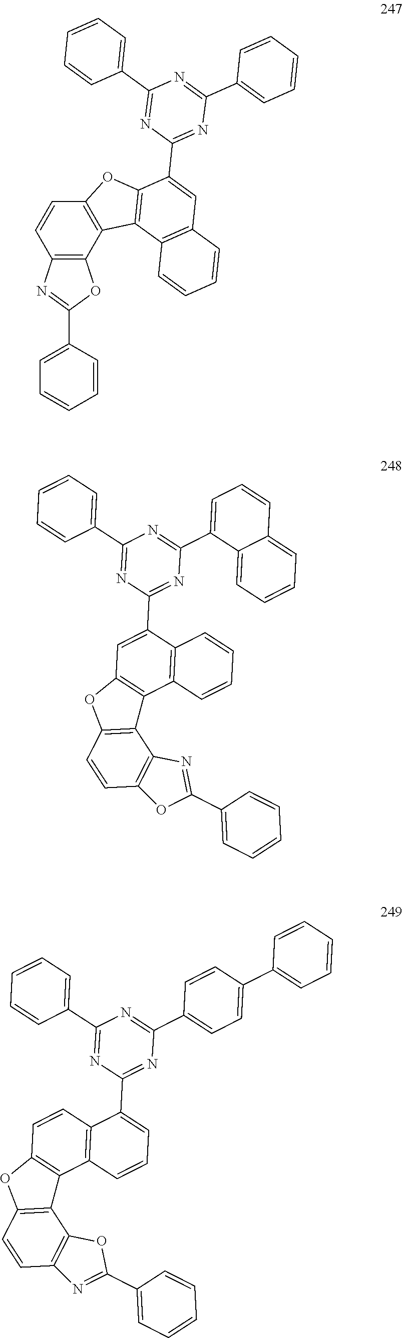 [SMILES: c1ccc(-c2nc(-c3ccccc3)nc(-c3cc4ccccc4c4c3oc3ccc5nc(-c6ccccc6)oc5c34)n2)cc1 ; c1ccc(-c2nc(-c3cccc4ccccc34)nc(-c3cc4oc5ccc6oc(-c7ccccc7)nc6c5c4c4ccccc34)n2)cc1 ; c1ccc(-c2ccc(-c3nc(-c4ccccc4)nc(-c4cccc5c4ccc4oc6ccc7nc(-c8ccccc8)oc7c6c45)n3)cc2)cc1]

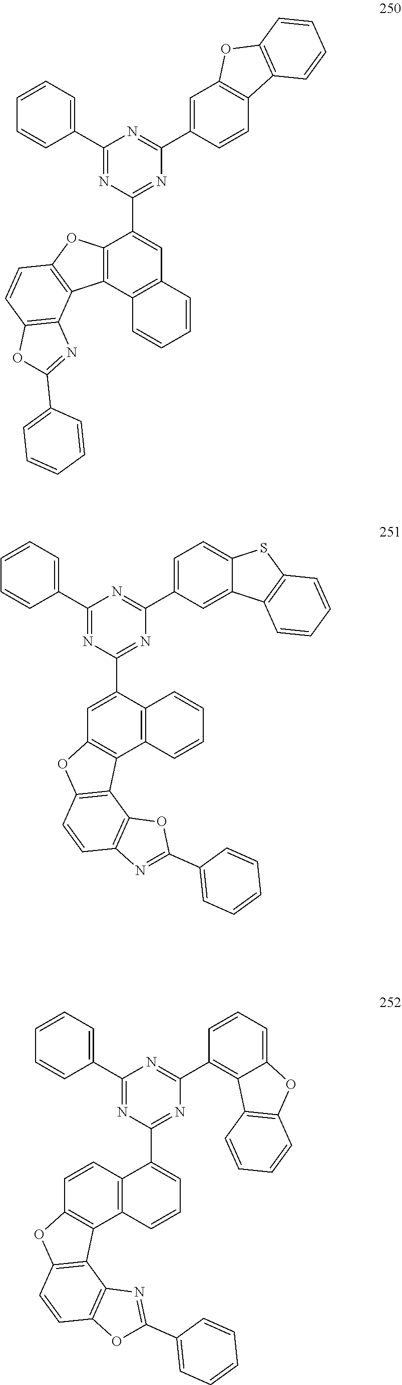 [SMILES: c1ccc(-c2nc(-c3ccc4c(c3)oc3ccccc34)nc(-c3cc4ccccc4c4c3oc3ccc5oc(-c6ccccc6)nc5c34)n2)cc1 ; c1ccc(-c2nc(-c3ccc4sc5ccccc5c4c3)nc(-c3cc4oc5ccc6nc(-c7ccccc7)oc6c5c4c4ccccc34)n2)cc1 ; c1ccc(-c2nc(-c3cccc4c3ccc3oc5ccc6oc(-c7ccccc7)nc6c5c34)nc(-c3cccc4oc5ccccc5c34)n2)cc1]

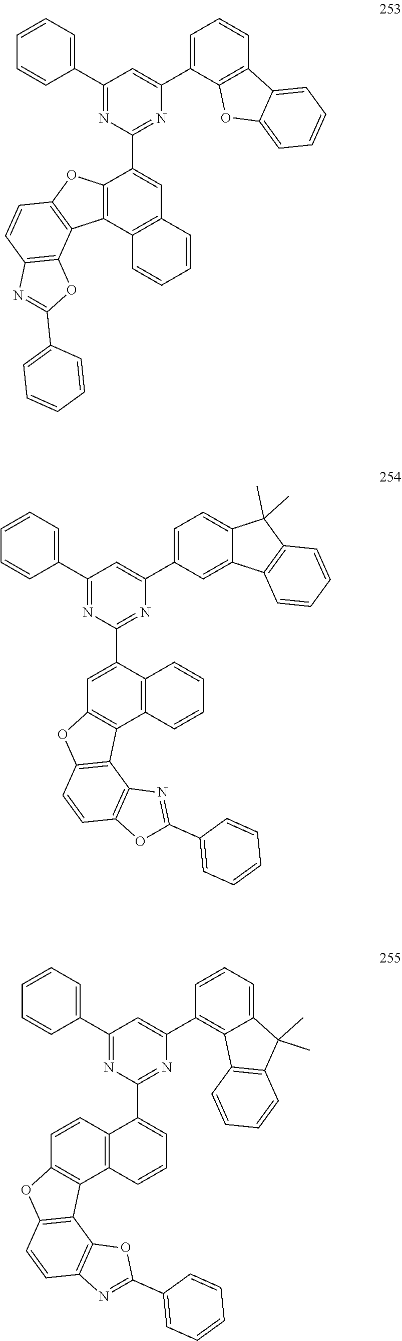 [SMILES: c1ccc(-c2cc(-c3cccc4c3oc3ccccc34)nc(-c3cc4ccccc4c4c3oc3ccc5nc(-c6ccccc6)oc5c34)n2)cc1 ; CC1(C)c2ccccc2-c2cc(-c3cc(-c4ccccc4)nc(-c4cc5oc6ccc7oc(-c8ccccc8)nc7c6c5c5ccccc45)n3)ccc21 ; CC1(C)c2ccccc2-c2c(-c3cc(-c4ccccc4)nc(-c4cccc5c4ccc4oc6ccc7nc(-c8ccccc8)oc7c6c45)n3)cccc21]

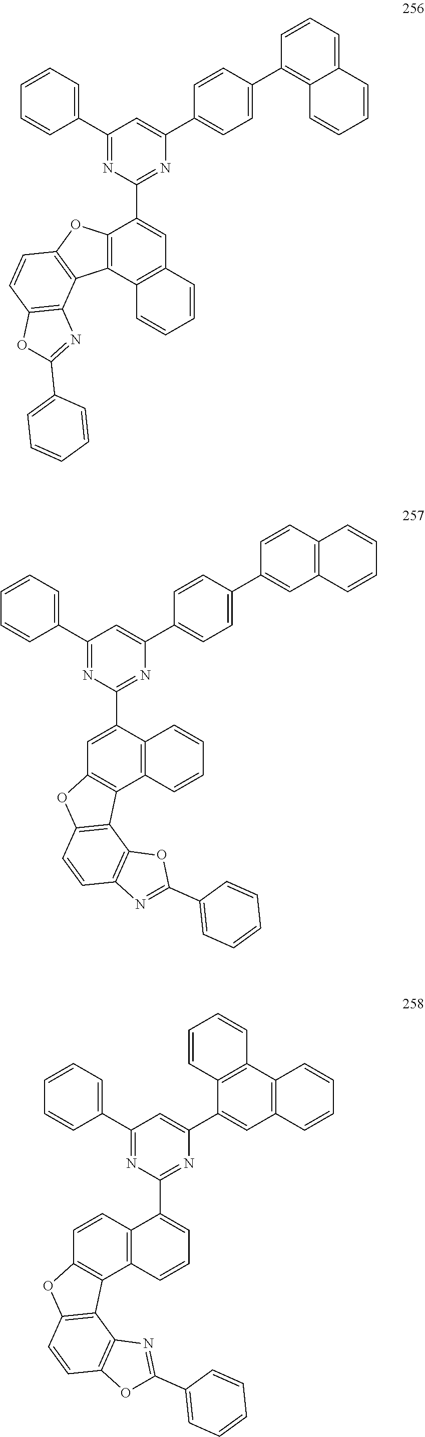 [SMILES: c1ccc(-c2cc(-c3ccc(-c4cccc5ccccc45)cc3)nc(-c3cc4ccccc4c4c3oc3ccc5oc(-c6ccccc6)nc5c34)n2)cc1 ; c1ccc(-c2cc(-c3ccc(-c4ccc5ccccc5c4)cc3)nc(-c3cc4oc5ccc6nc(-c7ccccc7)oc6c5c4c4ccccc34)n2)cc1 ; c1ccc(-c2cc(-c3cc4ccccc4c4ccccc34)nc(-c3cccc4c3ccc3oc5ccc6oc(-c7ccccc7)nc6c5c34)n2)cc1]

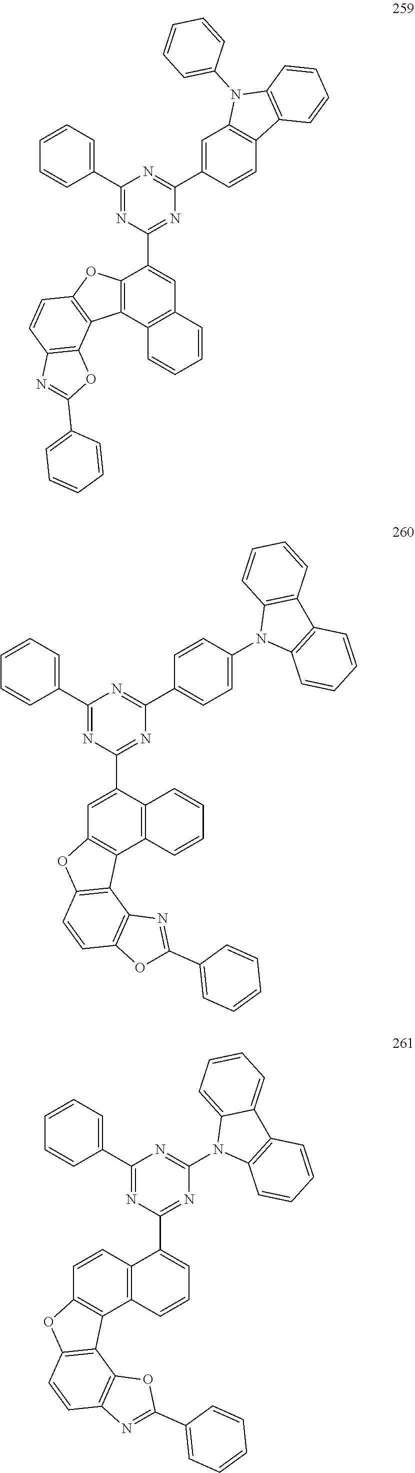

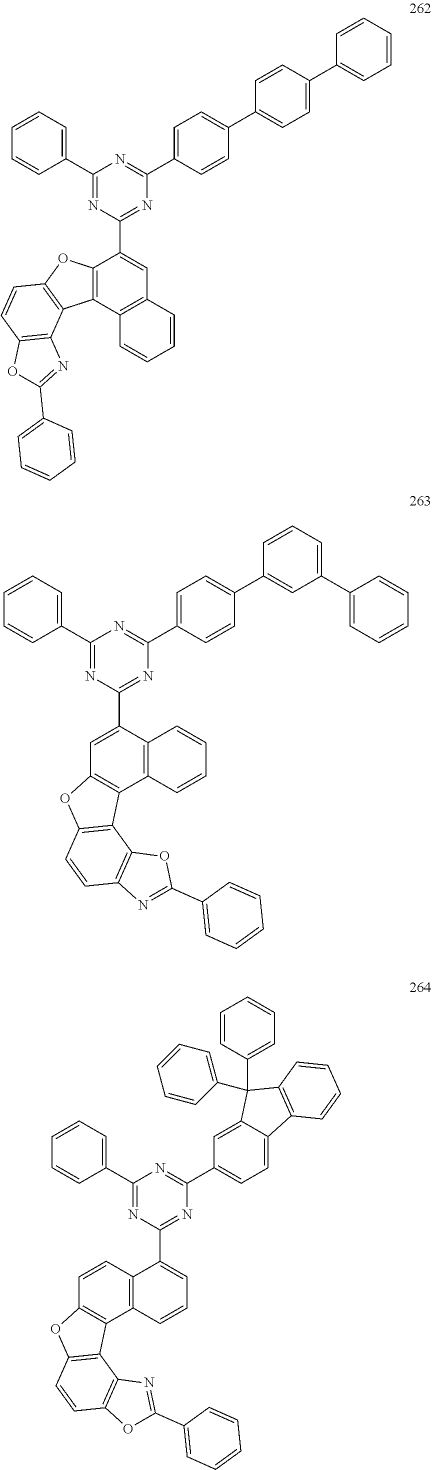 [SMILES: c1ccc(-c2ccc(-c3ccc(-c4nc(-c5ccccc5)nc(-c5cc6ccccc6c6c5oc5ccc7oc(-c8ccccc8)nc7c56)n4)cc3)cc2)cc1 ; c1ccc(-c2cccc(-c3ccc(-c4nc(-c5ccccc5)nc(-c5cc6oc7ccc8nc(-c9ccccc9)oc8c7c6c6ccccc56)n4)cc3)c2)cc1 ; c1ccc(-c2nc(-c3ccc4c(c3)C(c3ccccc3)(c3ccccc3)c3ccccc3-4)nc(-c3cccc4c3ccc3oc5ccc6oc(-c7ccccc7)nc6c5c34)n2)cc1]

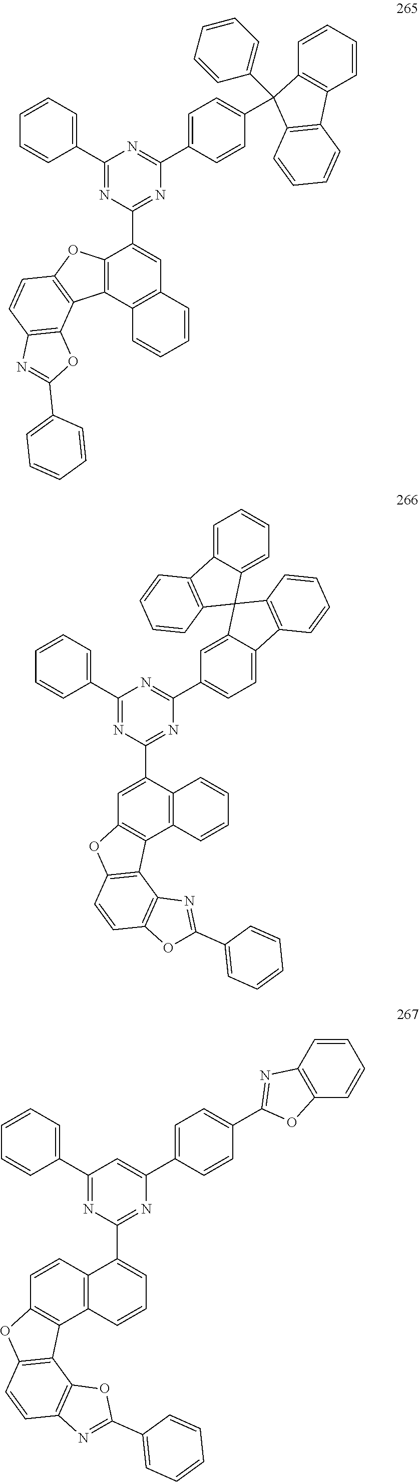 [SMILES: c1ccc(-c2nc(-c3ccc(C4(c5ccccc5)c5ccccc5-c5ccccc54)cc3)nc(-c3cc4ccccc4c4c3oc3ccc5nc(-c6ccccc6)oc5c34)n2)cc1 ; c1ccc(-c2nc(-c3ccc4c(c3)C3(c5ccccc5-c5ccccc53)c3ccccc3-4)nc(-c3cc4oc5ccc6oc(-c7ccccc7)nc6c5c4c4ccccc34)n2)cc1 ; c1ccc(-c2cc(-c3ccc(-c4nc5ccccc5o4)cc3)nc(-c3cccc4c3ccc3oc5ccc6nc(-c7ccccc7)oc6c5c34)n2)cc1]

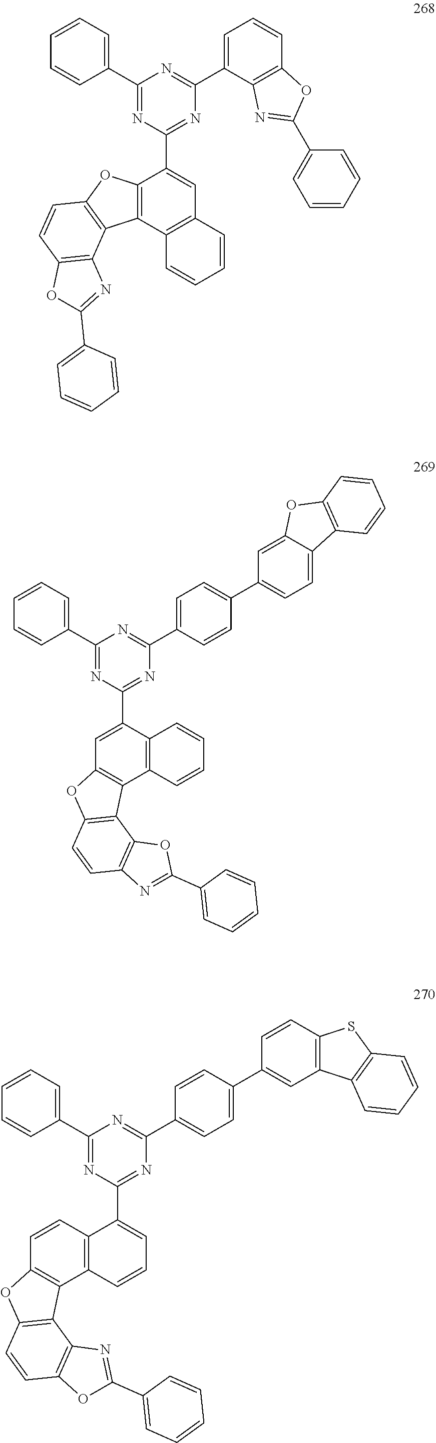 [SMILES: c1ccc(-c2nc(-c3cccc4oc(-c5ccccc5)nc34)nc(-c3cc4ccccc4c4c3oc3ccc5oc(-c6ccccc6)nc5c34)n2)cc1 ; c1ccc(-c2nc(-c3ccc(-c4ccc5c(c4)oc4ccccc45)cc3)nc(-c3cc4oc5ccc6nc(-c7ccccc7)oc6c5c4c4ccccc34)n2)cc1 ; c1ccc(-c2nc(-c3ccc(-c4ccc5sc6ccccc6c5c4)cc3)nc(-c3cccc4c3ccc3oc5ccc6oc(-c7ccccc7)nc6c5c34)n2)cc1]

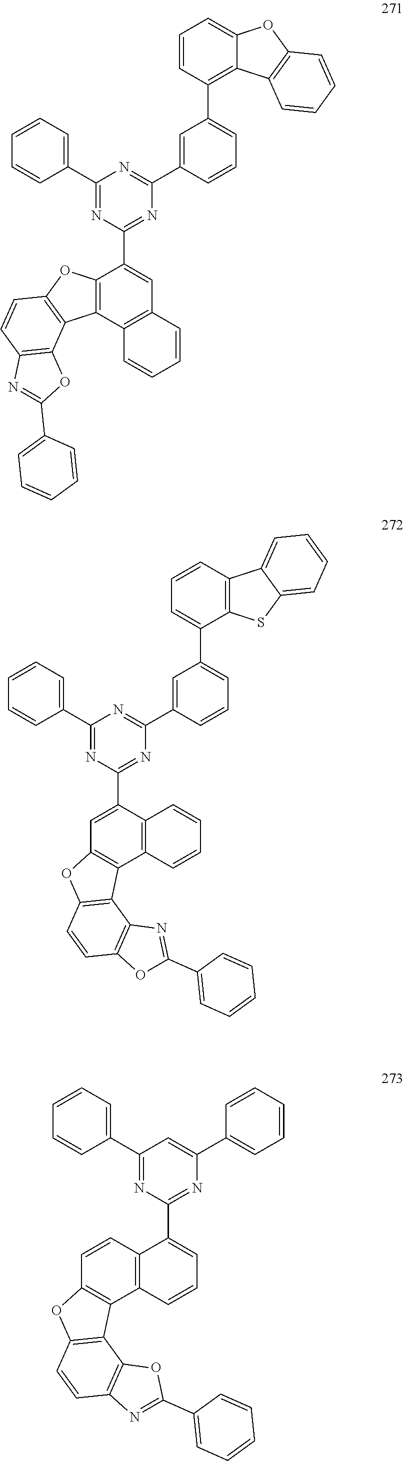 [SMILES: c1ccc(-c2nc(-c3cccc(-c4cccc5oc6ccccc6c45)c3)nc(-c3cc4ccccc4c4c3oc3ccc5nc(-c6ccccc6)oc5c34)n2)cc1 ; c1ccc(-c2nc(-c3cccc(-c4cccc5c4sc4ccccc45)c3)nc(-c3cc4oc5ccc6oc(-c7ccccc7)nc6c5c4c4ccccc34)n2)cc1 ; c1ccc(-c2cc(-c3ccccc3)nc(-c3cccc4c3ccc3oc5ccc6nc(-c7ccccc7)oc6c5c34)n2)cc1]

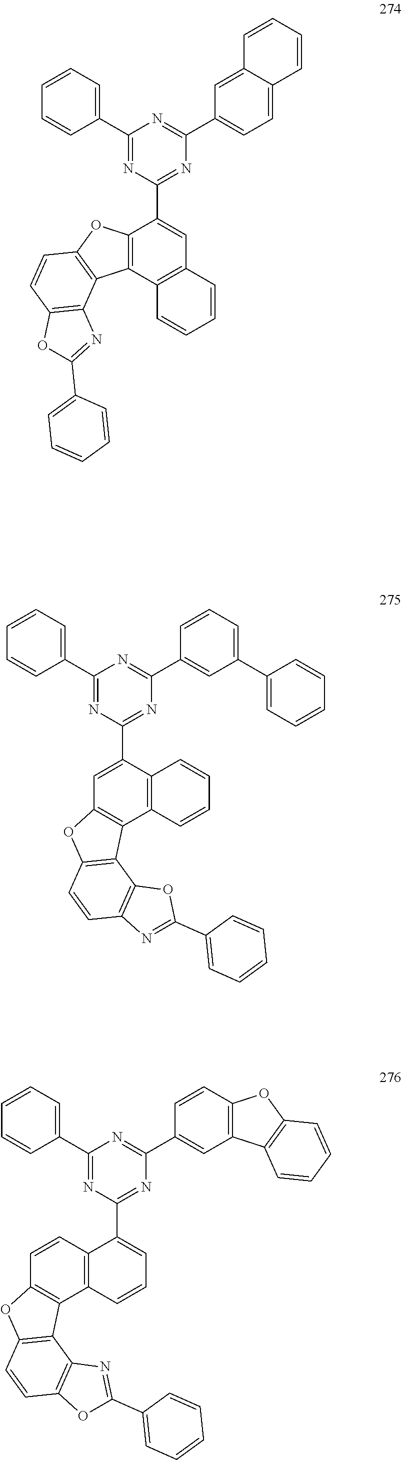 [SMILES: c1ccc(-c2nc(-c3ccc4ccccc4c3)nc(-c3cc4ccccc4c4c3oc3ccc5oc(-c6ccccc6)nc5c34)n2)cc1 ; c1ccc(-c2cccc(-c3nc(-c4ccccc4)nc(-c4cc5oc6ccc7nc(-c8ccccc8)oc7c6c5c5ccccc45)n3)c2)cc1 ; c1ccc(-c2nc(-c3ccc4oc5ccccc5c4c3)nc(-c3cccc4c3ccc3oc5ccc6oc(-c7ccccc7)nc6c5c34)n2)cc1]

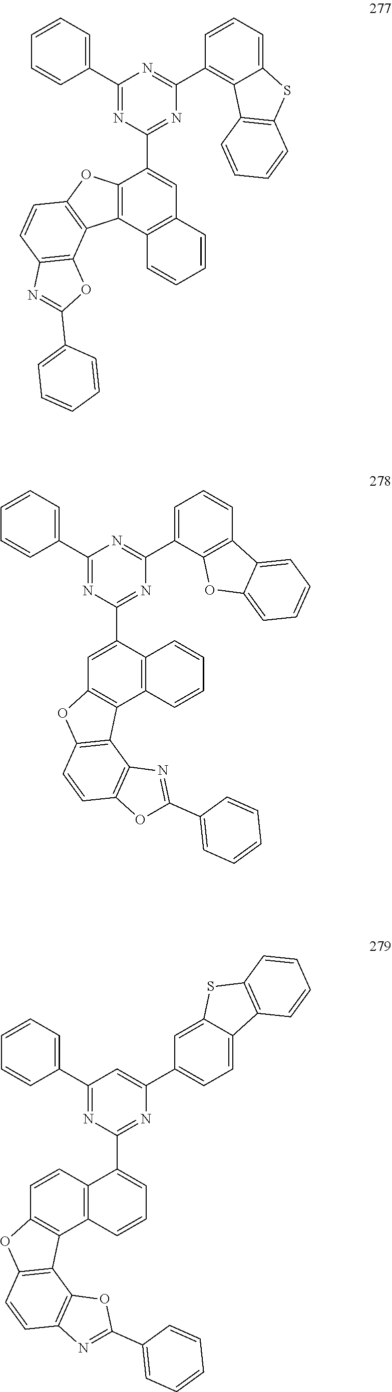 [SMILES: c1ccc(-c2nc(-c3cc4ccccc4c4c3oc3ccc5nc(-c6ccccc6)oc5c34)nc(-c3cccc4sc5ccccc5c34)n2)cc1 ; c1ccc(-c2nc(-c3cc4oc5ccc6oc(-c7ccccc7)nc6c5c4c4ccccc34)nc(-c3cccc4c3oc3ccccc34)n2)cc1 ; c1ccc(-c2cc(-c3ccc4c(c3)sc3ccccc34)nc(-c3cccc4c3ccc3oc5ccc6nc(-c7ccccc7)oc6c5c34)n2)cc1]

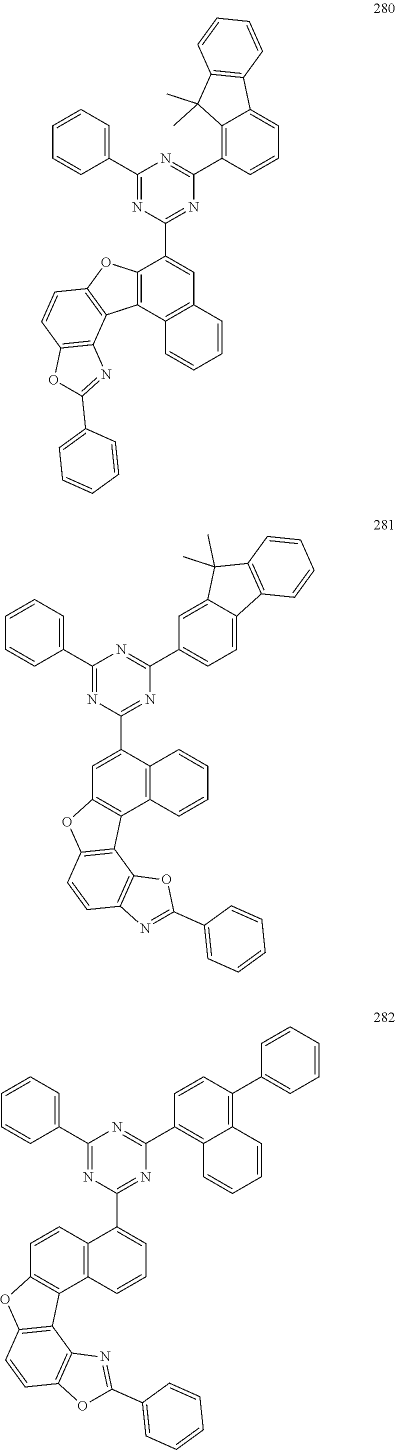 [SMILES: CC1(C)c2ccccc2-c2cccc(-c3nc(-c4ccccc4)nc(-c4cc5ccccc5c5c4oc4ccc6oc(-c7ccccc7)nc6c45)n3)c21 ; CC1(C)c2ccccc2-c2ccc(-c3nc(-c4ccccc4)nc(-c4cc5oc6ccc7nc(-c8ccccc8)oc7c6c5c5ccccc45)n3)cc21 ; c1ccc(-c2nc(-c3ccc(-c4ccccc4)c4ccccc34)nc(-c3cccc4c3ccc3oc5ccc6oc(-c7ccccc7)nc6c5c34)n2)cc1]

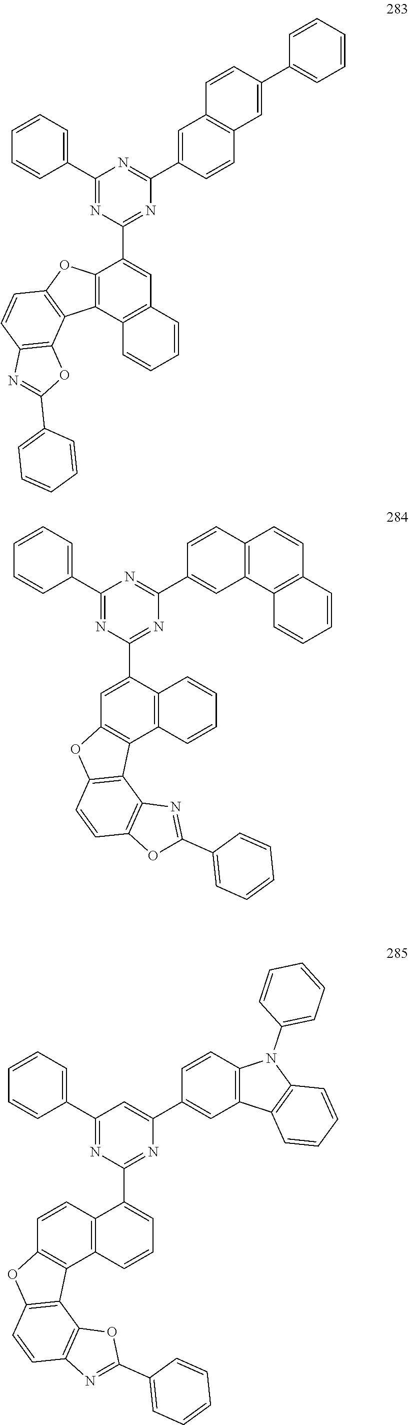

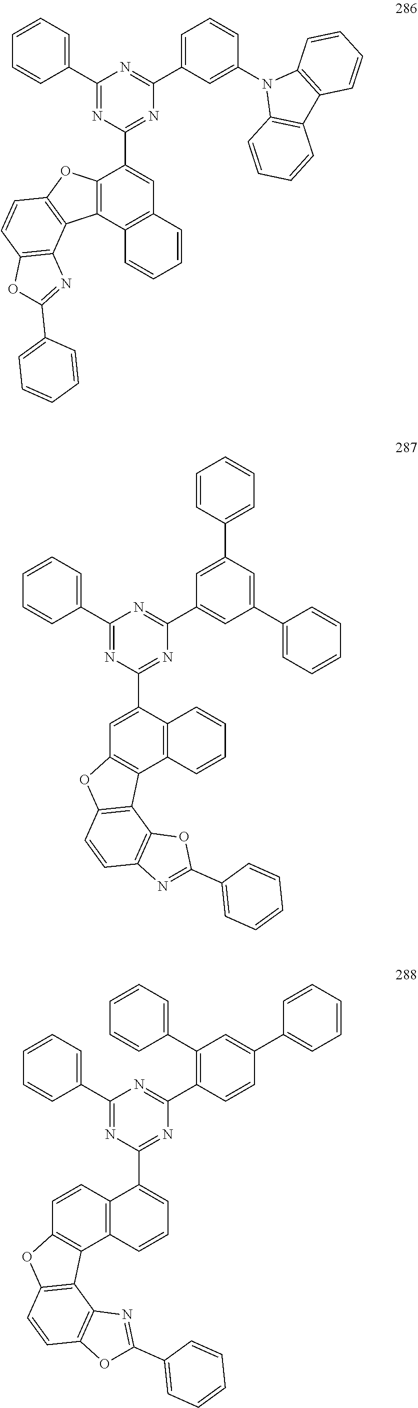 [SMILES: c1ccc(-c2nc(-c3cccc(-n4c5ccccc5c5ccccc54)c3)nc(-c3cc4ccccc4c4c3oc3ccc5oc(-c6ccccc6)nc5c34)n2)cc1 ; c1ccc(-c2cc(-c3ccccc3)cc(-c3nc(-c4ccccc4)nc(-c4cc5oc6ccc7nc(-c8ccccc8)oc7c6c5c5ccccc45)n3)c2)cc1 ; c1ccc(-c2ccc(-c3nc(-c4ccccc4)nc(-c4cccc5c4ccc4oc6ccc7oc(-c8ccccc8)nc7c6c45)n3)c(-c3ccccc3)c2)cc1]

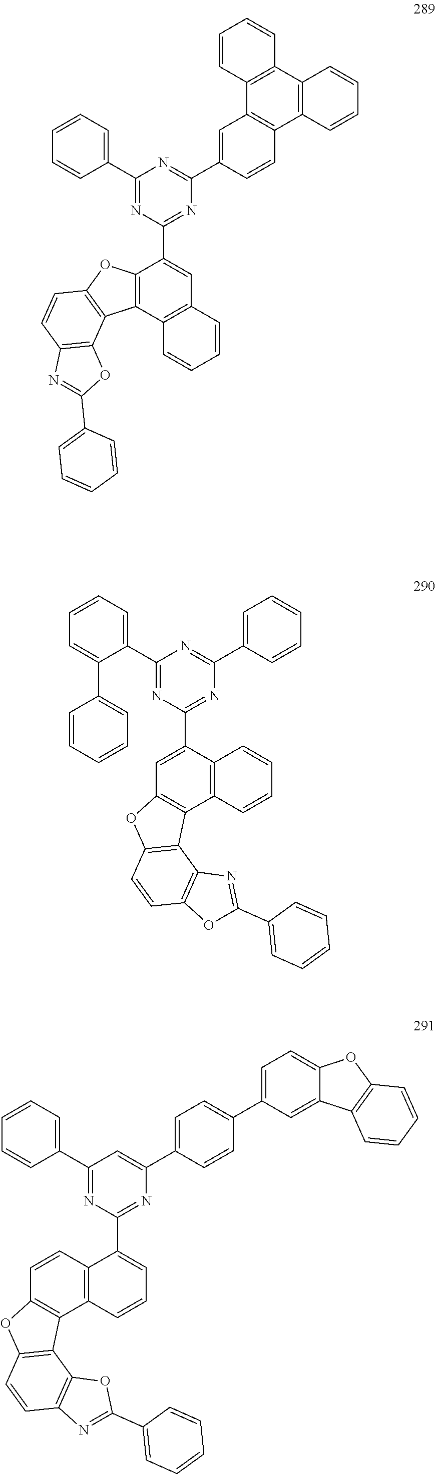 [SMILES: c1ccc(-c2nc(-c3ccc4c5ccccc5c5ccccc5c4c3)nc(-c3cc4ccccc4c4c3oc3ccc5nc(-c6ccccc6)oc5c34)n2)cc1 ; c1ccc(-c2nc(-c3ccccc3-c3ccccc3)nc(-c3cc4oc5ccc6oc(-c7ccccc7)nc6c5c4c4ccccc34)n2)cc1 ; c1ccc(-c2cc(-c3ccc(-c4ccc5oc6ccccc6c5c4)cc3)nc(-c3cccc4c3ccc3oc5ccc6nc(-c7ccccc7)oc6c5c34)n2)cc1]

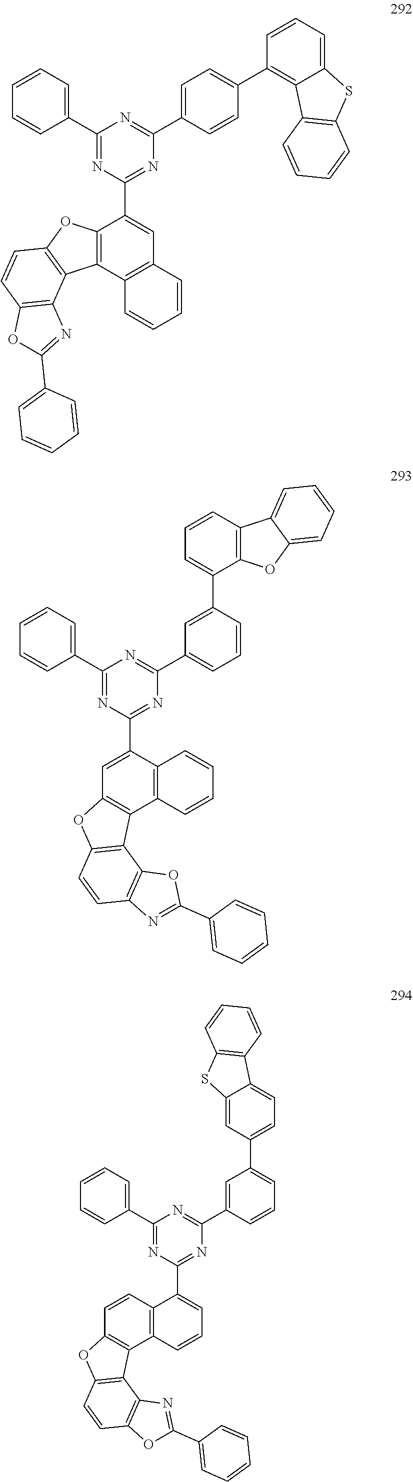 [SMILES: c1ccc(-c2nc(-c3ccc(-c4cccc5sc6ccccc6c45)cc3)nc(-c3cc4ccccc4c4c3oc3ccc5oc(-c6ccccc6)nc5c34)n2)cc1 ; c1ccc(-c2nc(-c3cccc(-c4cccc5c4oc4ccccc45)c3)nc(-c3cc4oc5ccc6nc(-c7ccccc7)oc6c5c4c4ccccc34)n2)cc1 ; c1ccc(-c2nc(-c3cccc(-c4ccc5c(c4)sc4ccccc45)c3)nc(-c3cccc4c3ccc3oc5ccc6oc(-c7ccccc7)nc6c5c34)n2)cc1]

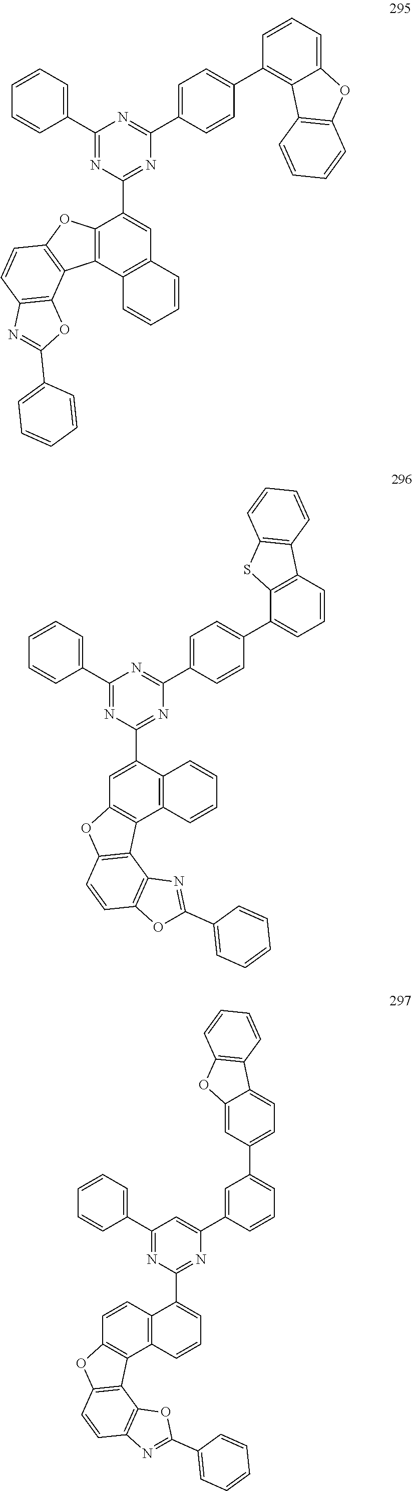 [SMILES: c1ccc(-c2nc(-c3ccc(-c4cccc5oc6ccccc6c45)cc3)nc(-c3cc4ccccc4c4c3oc3ccc5nc(-c6ccccc6)oc5c34)n2)cc1 ; c1ccc(-c2nc(-c3ccc(-c4cccc5c4sc4ccccc45)cc3)nc(-c3cc4oc5ccc6oc(-c7ccccc7)nc6c5c4c4ccccc34)n2)cc1 ; c1ccc(-c2cc(-c3cccc(-c4ccc5c(c4)oc4ccccc45)c3)nc(-c3cccc4c3ccc3oc5ccc6nc(-c7ccccc7)oc6c5c34)n2)cc1]

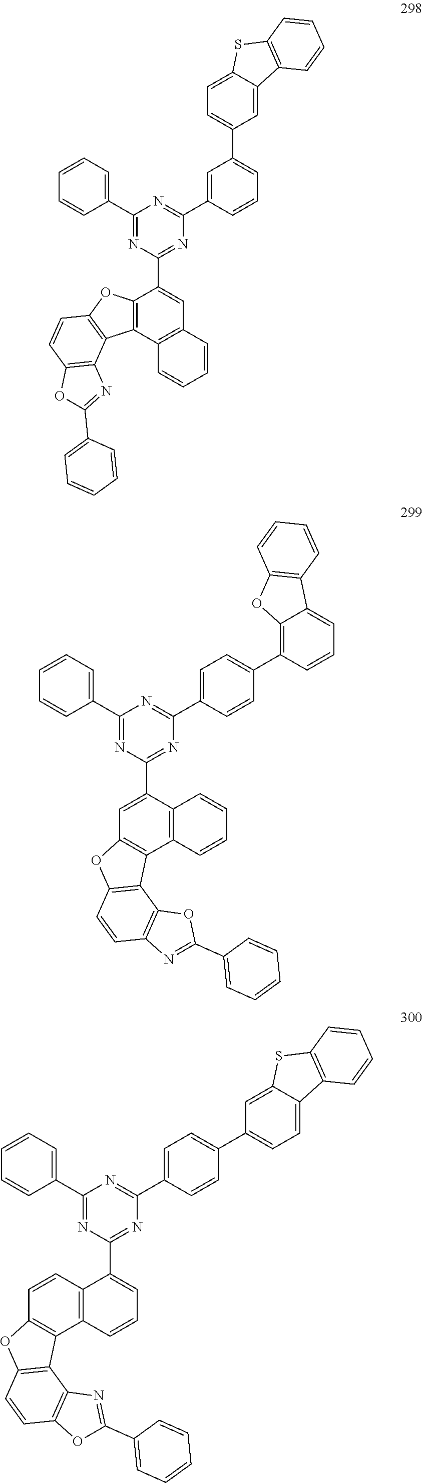 [SMILES: c1ccc(-c2nc(-c3cccc(-c4ccc5sc6ccccc6c5c4)c3)nc(-c3cc4ccccc4c4c3oc3ccc5oc(-c6ccccc6)nc5c34)n2)cc1 ; c1ccc(-c2nc(-c3ccc(-c4cccc5c4oc4ccccc45)cc3)nc(-c3cc4oc5ccc6nc(-c7ccccc7)oc6c5c4c4ccccc34)n2)cc1 ; c1ccc(-c2nc(-c3ccc(-c4ccc5c(c4)sc4ccccc45)cc3)nc(-c3cccc4c3ccc3oc5ccc6oc(-c7ccccc7)nc6c5c34)n2)cc1]

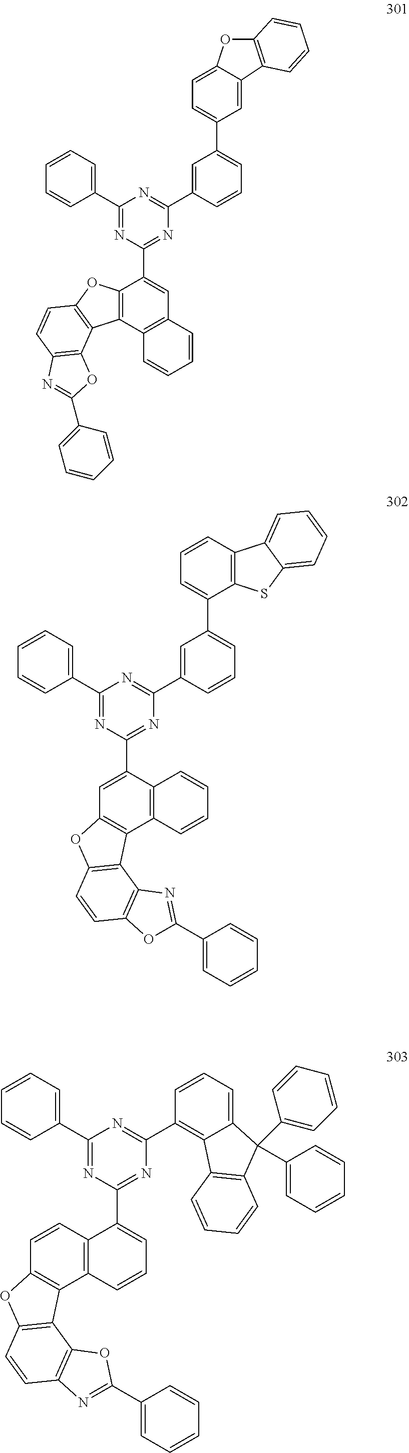 [SMILES: c1ccc(-c2nc(-c3cccc(-c4ccc5oc6ccccc6c5c4)c3)nc(-c3cc4ccccc4c4c3oc3ccc5nc(-c6ccccc6)oc5c34)n2)cc1 ; c1ccc(-c2nc(-c3cccc(-c4cccc5c4sc4ccccc45)c3)nc(-c3cc4oc5ccc6oc(-c7ccccc7)nc6c5c4c4ccccc34)n2)cc1 ; c1ccc(-c2nc(-c3cccc4c3-c3ccccc3C4(c3ccccc3)c3ccccc3)nc(-c3cccc4c3ccc3oc5ccc6nc(-c7ccccc7)oc6c5c34)n2)cc1]

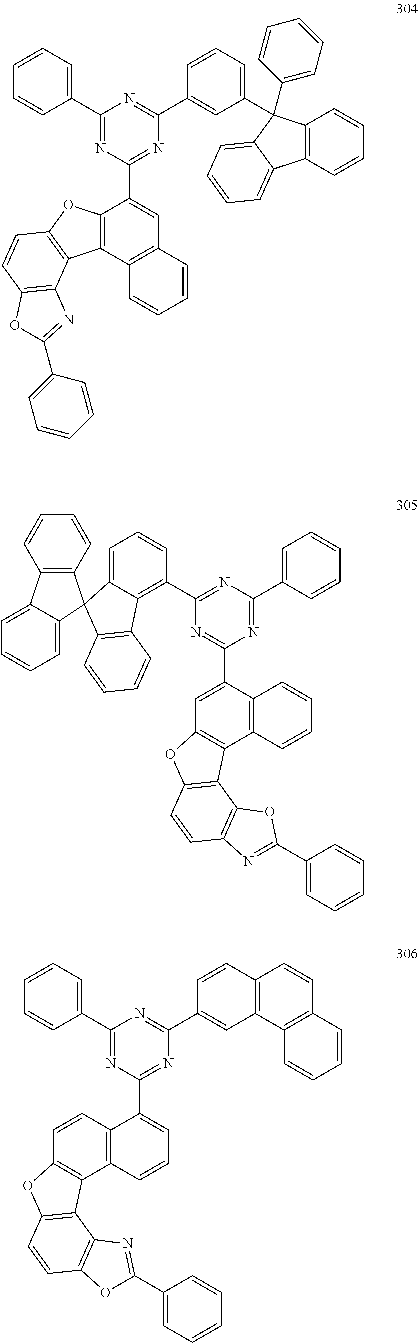 [SMILES: c1ccc(-c2nc(-c3cccc(C4(c5ccccc5)c5ccccc5-c5ccccc54)c3)nc(-c3cc4ccccc4c4c3oc3ccc5oc(-c6ccccc6)nc5c34)n2)cc1 ; c1ccc(-c2nc(-c3cccc4c3-c3ccccc3C43c4ccccc4-c4ccccc43)nc(-c3cc4oc5ccc6nc(-c7ccccc7)oc6c5c4c4ccccc34)n2)cc1 ; c1ccc(-c2nc(-c3ccc4ccc5ccccc5c4c3)nc(-c3cccc4c3ccc3oc5ccc6oc(-c7ccccc7)nc6c5c34)n2)cc1]

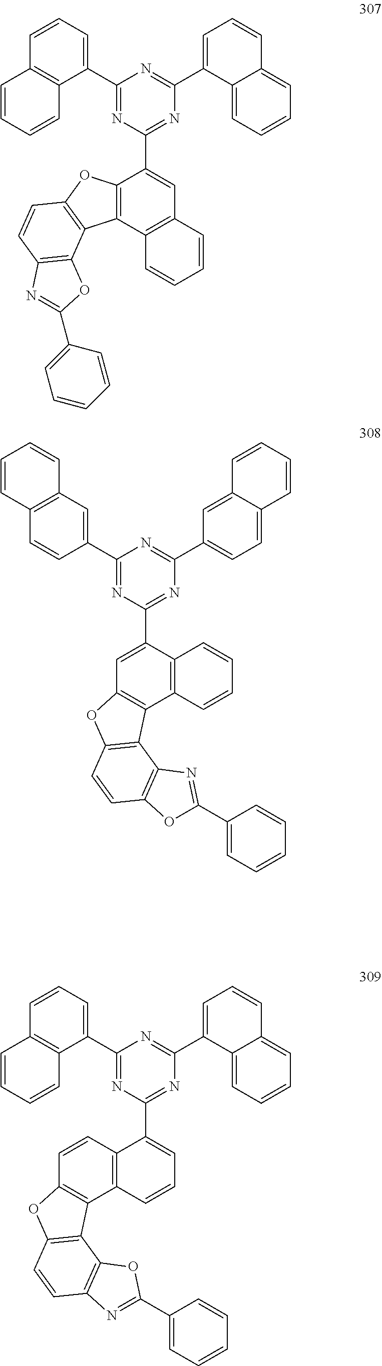 [SMILES: c1ccc(-c2nc3ccc4oc5c(-c6nc(-c7cccc8ccccc78)nc(-c7cccc8ccccc78)n6)cc6ccccc6c5c4c3o2)cc1 ; c1ccc(-c2nc3c(ccc4oc5cc(-c6nc(-c7ccc8ccccc8c7)nc(-c7ccc8ccccc8c7)n6)c6ccccc6c5c43)o2)cc1 ; c1ccc(-c2nc3ccc4oc5ccc6c(-c7nc(-c8cccc9ccccc89)nc(-c8cccc9ccccc89)n7)cccc6c5c4c3o2)cc1]

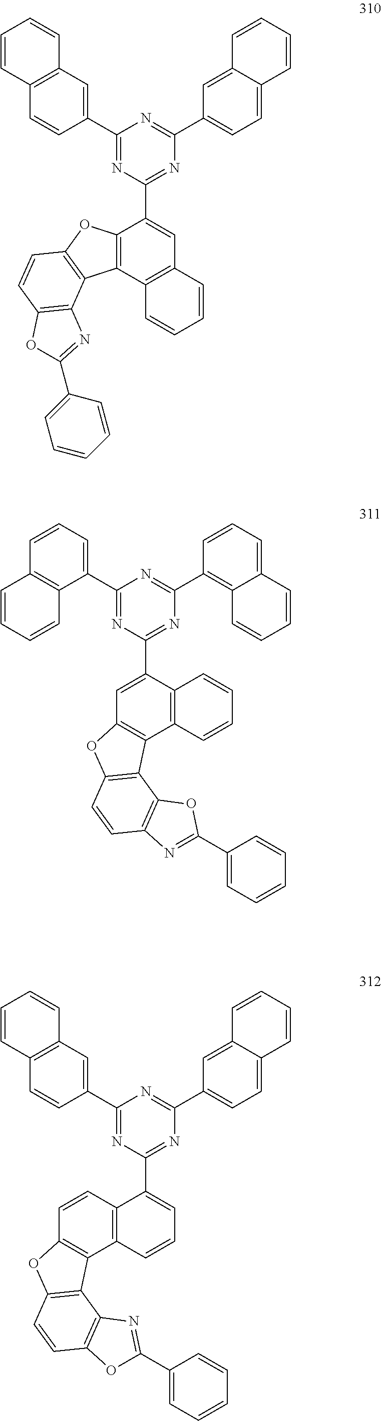 [SMILES: c1ccc(-c2nc3c(ccc4oc5c(-c6nc(-c7ccc8ccccc8c7)nc(-c7ccc8ccccc8c7)n6)cc6ccccc6c5c43)o2)cc1 ; c1ccc(-c2nc3ccc4oc5cc(-c6nc(-c7cccc8ccccc78)nc(-c7cccc8ccccc78)n6)c6ccccc6c5c4c3o2)cc1 ; c1ccc(-c2nc3c(ccc4oc5ccc6c(-c7nc(-c8ccc9ccccc9c8)nc(-c8ccc9ccccc9c8)n7)cccc6c5c43)o2)cc1]

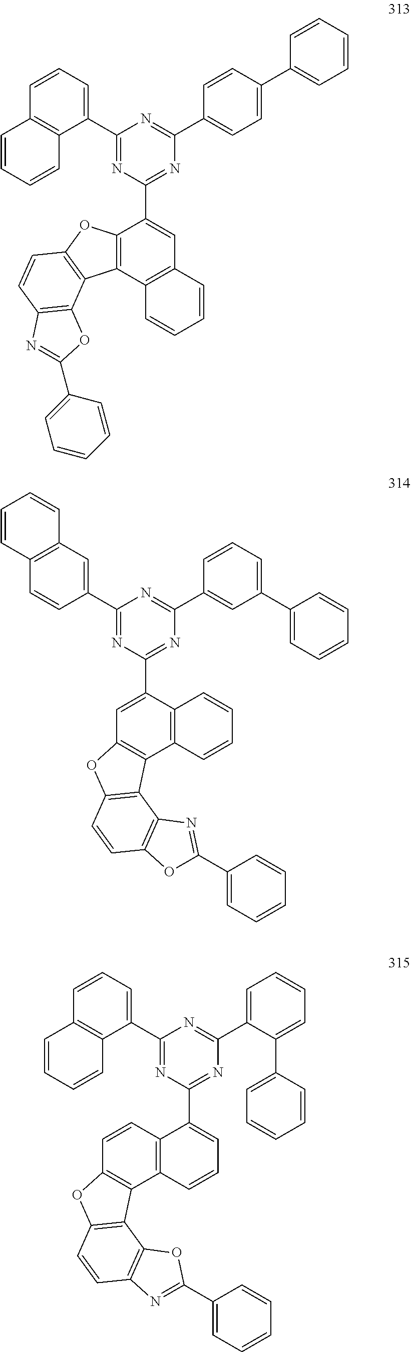 [SMILES: c1ccc(-c2ccc(-c3nc(-c4cccc5ccccc45)nc(-c4cc5ccccc5c5c4oc4ccc6nc(-c7ccccc7)oc6c45)n3)cc2)cc1 ; c1ccc(-c2cccc(-c3nc(-c4ccc5ccccc5c4)nc(-c4cc5oc6ccc7oc(-c8ccccc8)nc7c6c5c5ccccc45)n3)c2)cc1 ; c1ccc(-c2nc3ccc4oc5ccc6c(-c7nc(-c8ccccc8-c8ccccc8)nc(-c8cccc9ccccc89)n7)cccc6c5c4c3o2)cc1]

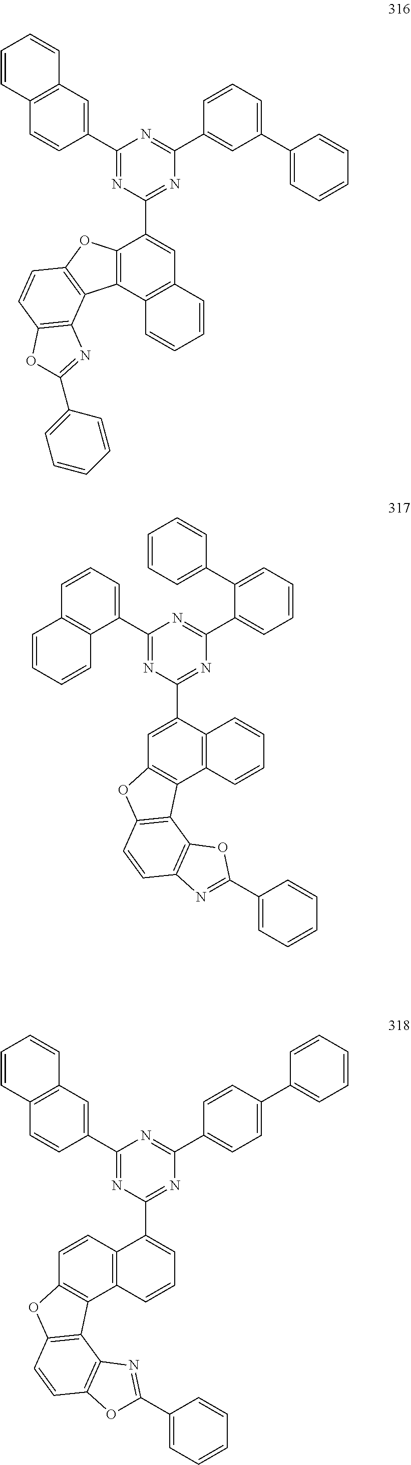 [SMILES: c1ccc(-c2cccc(-c3nc(-c4ccc5ccccc5c4)nc(-c4cc5ccccc5c5c4oc4ccc6oc(-c7ccccc7)nc6c45)n3)c2)cc1 ; c1ccc(-c2nc3ccc4oc5cc(-c6nc(-c7ccccc7-c7ccccc7)nc(-c7cccc8ccccc78)n6)c6ccccc6c5c4c3o2)cc1 ; c1ccc(-c2ccc(-c3nc(-c4ccc5ccccc5c4)nc(-c4cccc5c4ccc4oc6ccc7oc(-c8ccccc8)nc7c6c45)n3)cc2)cc1]

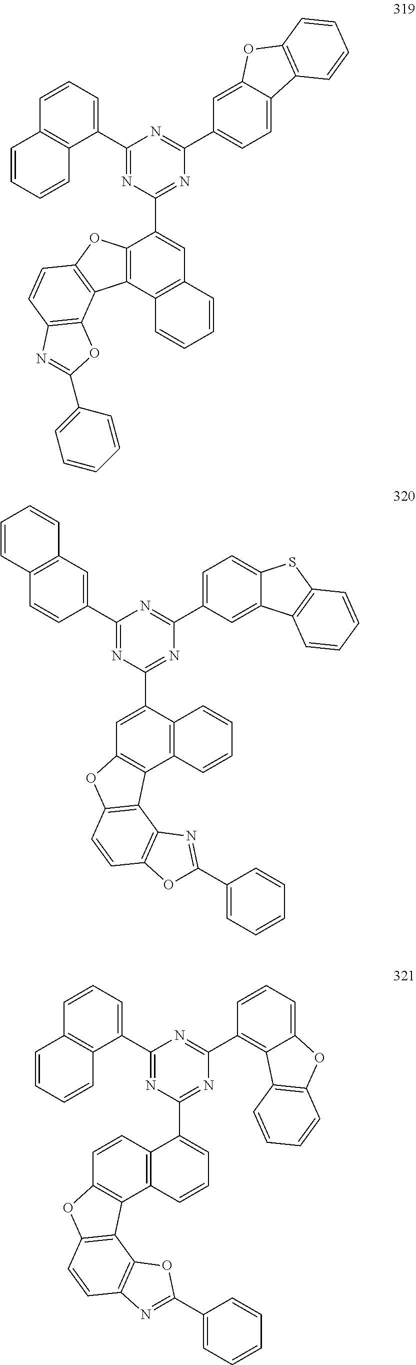 [SMILES: c1ccc(-c2nc3ccc4oc5c(-c6nc(-c7ccc8c(c7)oc7ccccc78)nc(-c7cccc8ccccc78)n6)cc6ccccc6c5c4c3o2)cc1 ; c1ccc(-c2nc3c(ccc4oc5cc(-c6nc(-c7ccc8ccccc8c7)nc(-c7ccc8sc9ccccc9c8c7)n6)c6ccccc6c5c43)o2)cc1 ; c1ccc(-c2nc3ccc4oc5ccc6c(-c7nc(-c8cccc9ccccc89)nc(-c8cccc9oc%10ccccc%10c89)n7)cccc6c5c4c3o2)cc1]

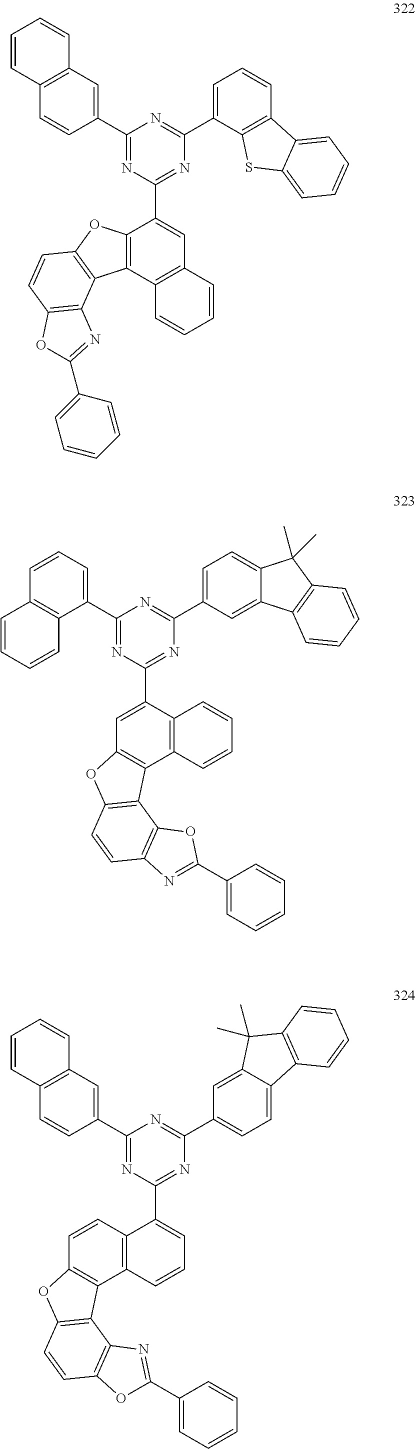 [SMILES: c1ccc(-c2nc3c(ccc4oc5c(-c6nc(-c7ccc8ccccc8c7)nc(-c7cccc8c7sc7ccccc78)n6)cc6ccccc6c5c43)o2)cc1 ; CC1(C)c2ccccc2-c2cc(-c3nc(-c4cccc5ccccc45)nc(-c4cc5oc6ccc7nc(-c8ccccc8)oc7c6c5c5ccccc45)n3)ccc21 ; CC1(C)c2ccccc2-c2ccc(-c3nc(-c4ccc5ccccc5c4)nc(-c4cccc5c4ccc4oc6ccc7oc(-c8ccccc8)nc7c6c45)n3)cc21]

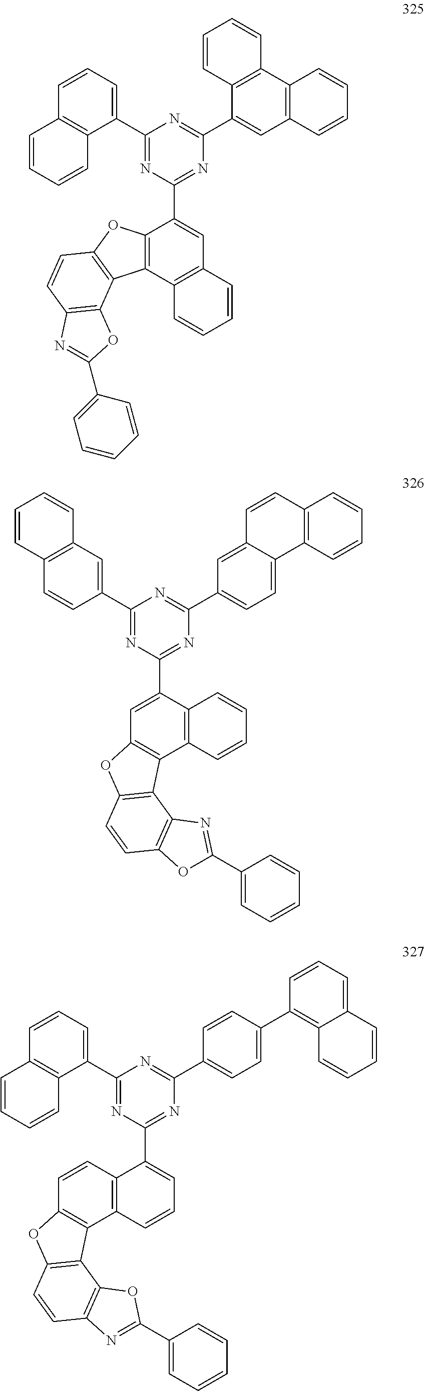 [SMILES: c1ccc(-c2nc3ccc4oc5c(-c6nc(-c7cccc8ccccc78)nc(-c7cc8ccccc8c8ccccc78)n6)cc6ccccc6c5c4c3o2)cc1 ; c1ccc(-c2nc3c(ccc4oc5cc(-c6nc(-c7ccc8ccccc8c7)nc(-c7ccc8c(ccc9ccccc98)c7)n6)c6ccccc6c5c43)o2)cc1 ; c1ccc(-c2nc3ccc4oc5ccc6c(-c7nc(-c8ccc(-c9cccc%10ccccc9%10)cc8)nc(-c8cccc9ccccc89)n7)cccc6c5c4c3o2)cc1]

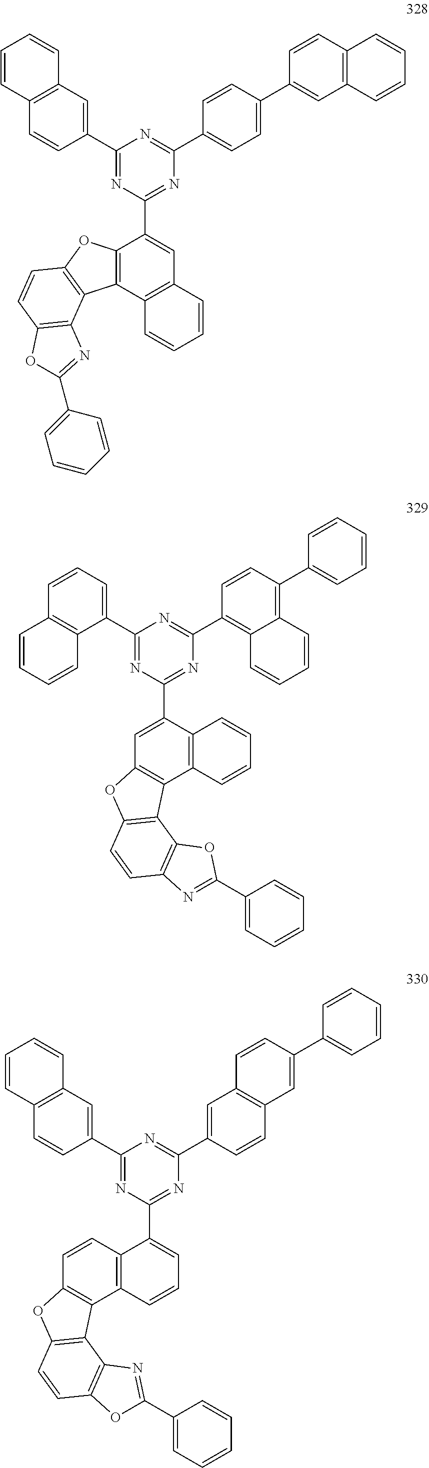 [SMILES: c1ccc(-c2nc3c(ccc4oc5c(-c6nc(-c7ccc(-c8ccc9ccccc9c8)cc7)nc(-c7ccc8ccccc8c7)n6)cc6ccccc6c5c43)o2)cc1 ; c1ccc(-c2nc3ccc4oc5cc(-c6nc(-c7cccc8ccccc78)nc(-c7ccc(-c8ccccc8)c8ccccc78)n6)c6ccccc6c5c4c3o2)cc1 ; c1ccc(-c2ccc3cc(-c4nc(-c5ccc6ccccc6c5)nc(-c5cccc6c5ccc5oc7ccc8oc(-c9ccccc9)nc8c7c56)n4)ccc3c2)cc1]

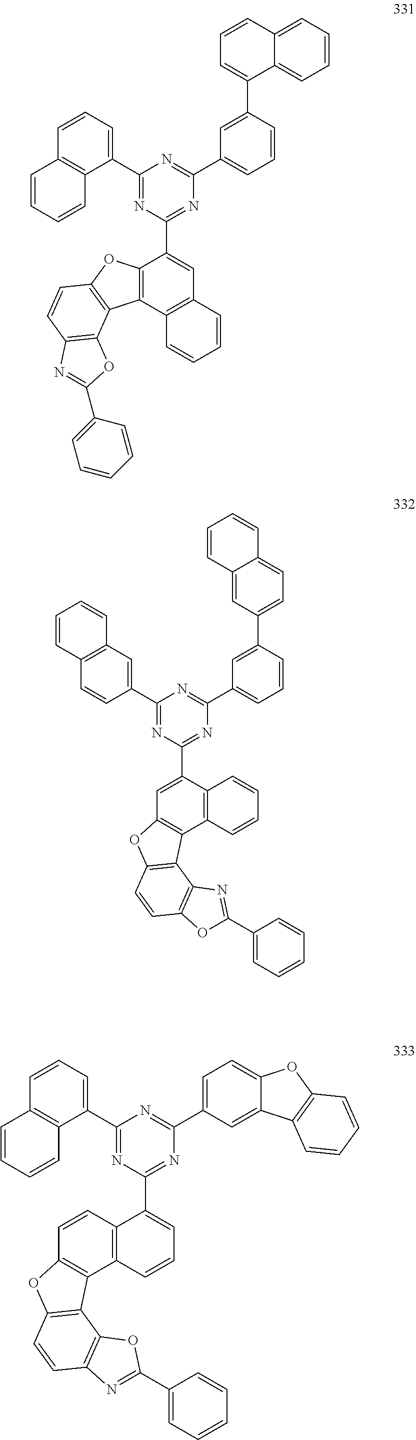 [SMILES: c1ccc(-c2nc3ccc4oc5c(-c6nc(-c7cccc(-c8cccc9ccccc89)c7)nc(-c7cccc8ccccc78)n6)cc6ccccc6c5c4c3o2)cc1 ; c1ccc(-c2nc3c(ccc4oc5cc(-c6nc(-c7cccc(-c8ccc9ccccc9c8)c7)nc(-c7ccc8ccccc8c7)n6)c6ccccc6c5c43)o2)cc1 ; c1ccc(-c2nc3ccc4oc5ccc6c(-c7nc(-c8ccc9oc%10ccccc%10c9c8)nc(-c8cccc9ccccc89)n7)cccc6c5c4c3o2)cc1]

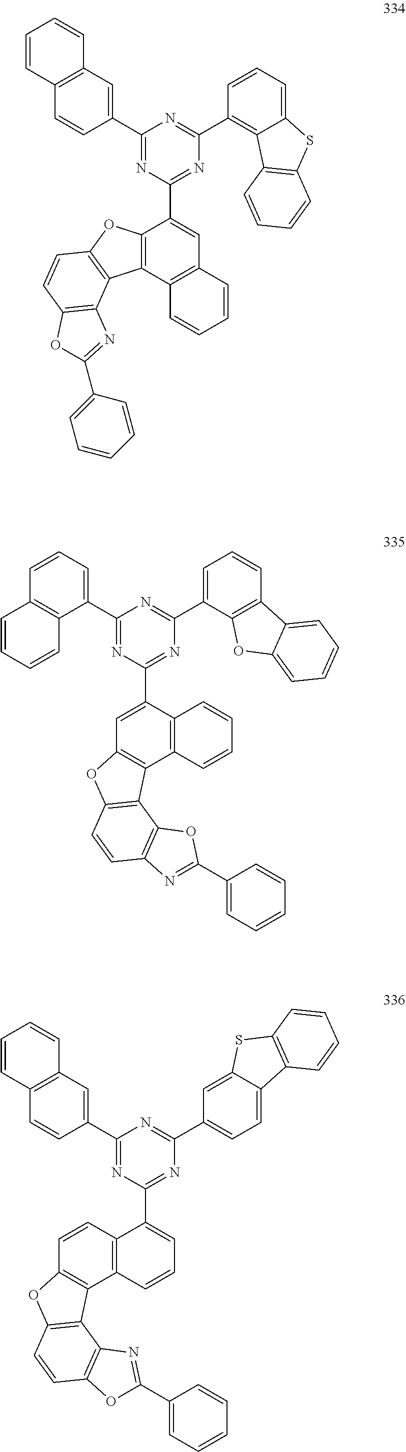 [SMILES: c1ccc(-c2nc3c(ccc4oc5c(-c6nc(-c7ccc8ccccc8c7)nc(-c7cccc8sc9ccccc9c78)n6)cc6ccccc6c5c43)o2)cc1 ; c1ccc(-c2nc3ccc4oc5cc(-c6nc(-c7cccc8ccccc78)nc(-c7cccc8c7oc7ccccc78)n6)c6ccccc6c5c4c3o2)cc1 ; c1ccc(-c2nc3c(ccc4oc5ccc6c(-c7nc(-c8ccc9ccccc9c8)nc(-c8ccc9c(c8)sc8ccccc89)n7)cccc6c5c43)o2)cc1]

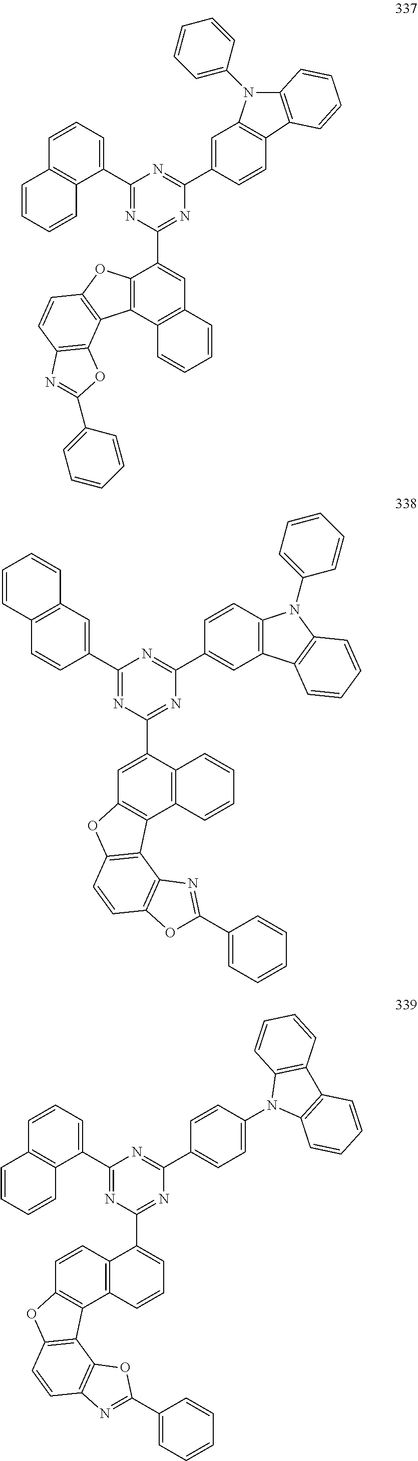

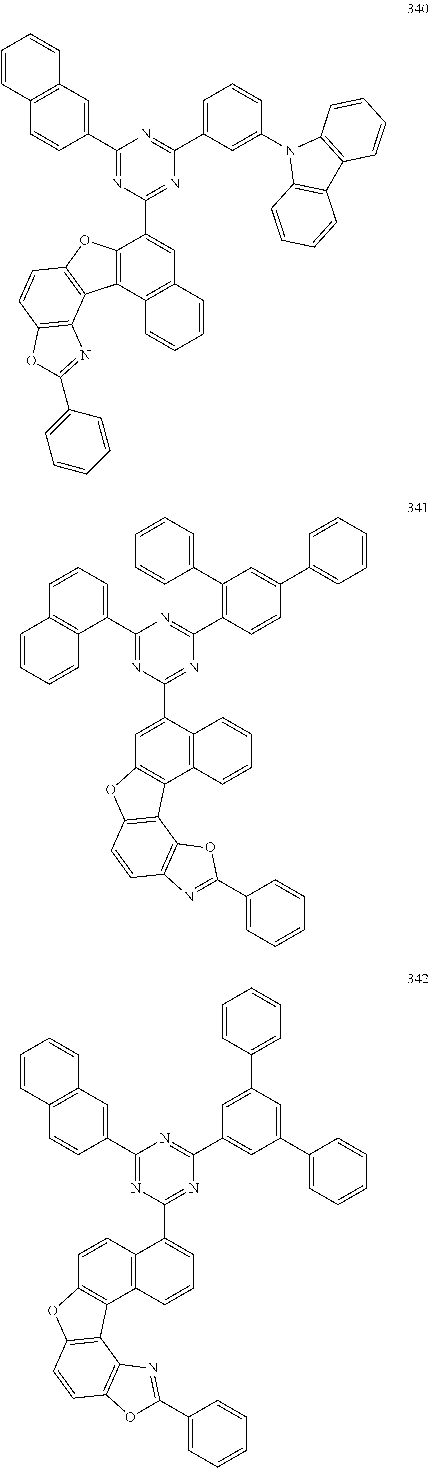 [SMILES: c1ccc(-c2nc3c(ccc4oc5c(-c6nc(-c7cccc(-n8c9ccccc9c9ccccc98)c7)nc(-c7ccc8ccccc8c7)n6)cc6ccccc6c5c43)o2)cc1 ; c1ccc(-c2ccc(-c3nc(-c4cccc5ccccc45)nc(-c4cc5oc6ccc7nc(-c8ccccc8)oc7c6c5c5ccccc45)n3)c(-c3ccccc3)c2)cc1 ; c1ccc(-c2cc(-c3ccccc3)cc(-c3nc(-c4ccc5ccccc5c4)nc(-c4cccc5c4ccc4oc6ccc7oc(-c8ccccc8)nc7c6c45)n3)c2)cc1]

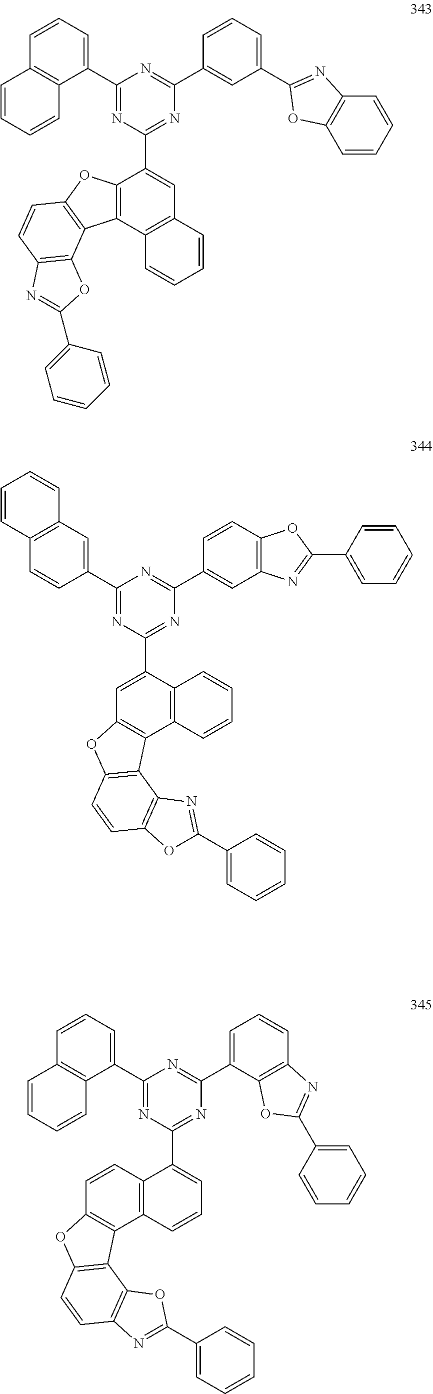 [SMILES: c1ccc(-c2nc3ccc4oc5c(-c6nc(-c7cccc(-c8nc9ccccc9o8)c7)nc(-c7cccc8ccccc78)n6)cc6ccccc6c5c4c3o2)cc1 ; c1ccc(-c2nc3cc(-c4nc(-c5ccc6ccccc6c5)nc(-c5cc6oc7ccc8oc(-c9ccccc9)nc8c7c6c6ccccc56)n4)ccc3o2)cc1 ; c1ccc(-c2nc3cccc(-c4nc(-c5cccc6ccccc56)nc(-c5cccc6c5ccc5oc7ccc8nc(-c9ccccc9)oc8c7c56)n4)c3o2)cc1]

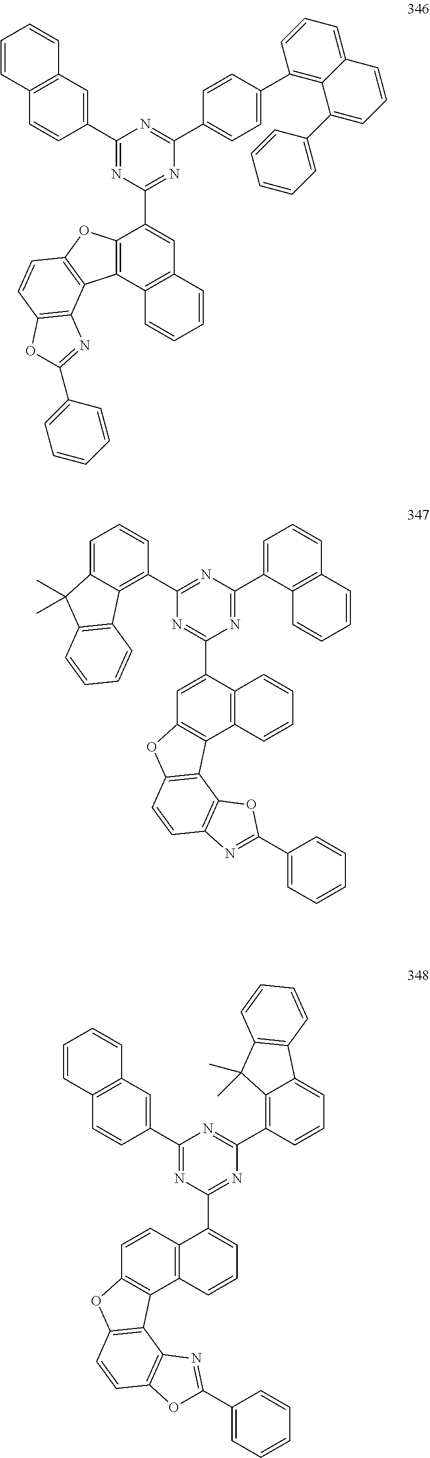 [SMILES: c1ccc(-c2nc3c(ccc4oc5c(-c6nc(-c7ccc(-c8cccc9cccc(-c%10ccccc%10)c89)cc7)nc(-c7ccc8ccccc8c7)n6)cc6ccccc6c5c43)o2)cc1 ; CC1(C)c2ccccc2-c2c(-c3nc(-c4cccc5ccccc45)nc(-c4cc5oc6ccc7nc(-c8ccccc8)oc7c6c5c5ccccc45)n3)cccc21 ; CC1(C)c2ccccc2-c2cccc(-c3nc(-c4ccc5ccccc5c4)nc(-c4cccc5c4ccc4oc6ccc7oc(-c8ccccc8)nc7c6c45)n3)c21]

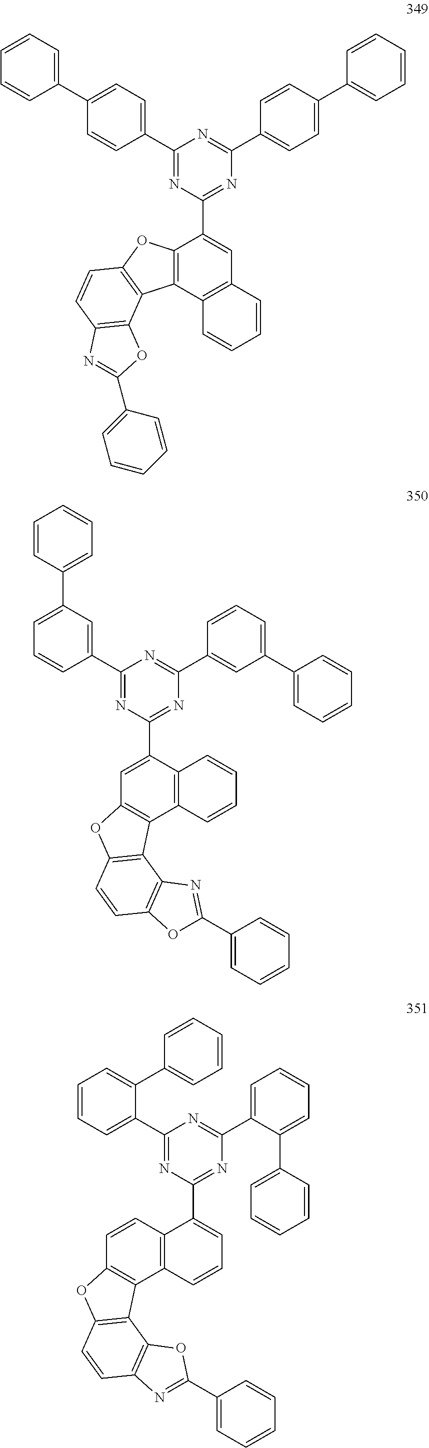 [SMILES: c1ccc(-c2ccc(-c3nc(-c4ccc(-c5ccccc5)cc4)nc(-c4cc5ccccc5c5c4oc4ccc6nc(-c7ccccc7)oc6c45)n3)cc2)cc1 ; c1ccc(-c2cccc(-c3nc(-c4cccc(-c5ccccc5)c4)nc(-c4cc5oc6ccc7oc(-c8ccccc8)nc7c6c5c5ccccc45)n3)c2)cc1 ; c1ccc(-c2nc3ccc4oc5ccc6c(-c7nc(-c8ccccc8-c8ccccc8)nc(-c8ccccc8-c8ccccc8)n7)cccc6c5c4c3o2)cc1]

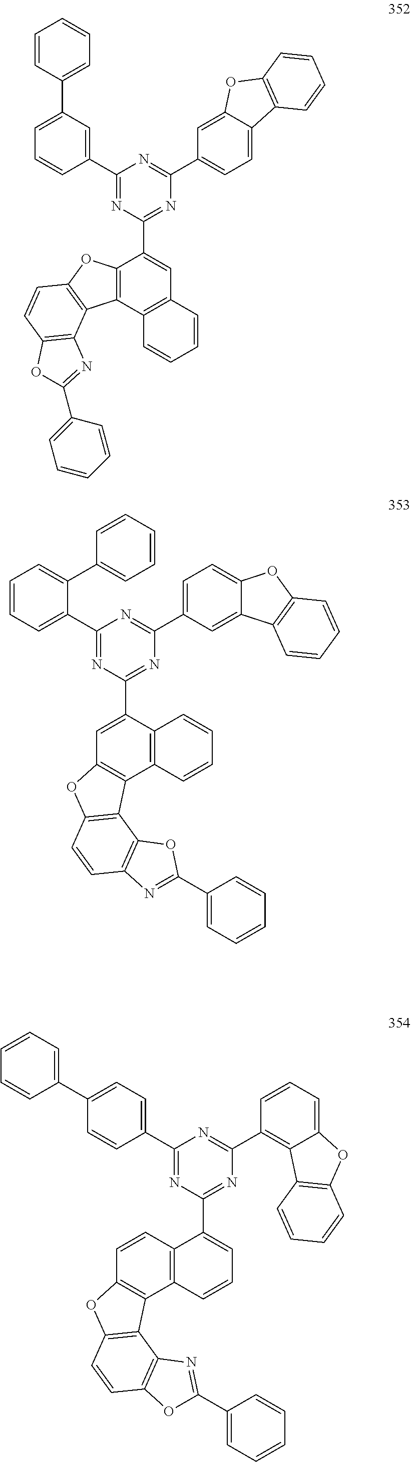 [SMILES: c1ccc(-c2cccc(-c3nc(-c4ccc5c(c4)oc4ccccc45)nc(-c4cc5ccccc5c5c4oc4ccc6oc(-c7ccccc7)nc6c45)n3)c2)cc1 ; c1ccc(-c2nc3ccc4oc5cc(-c6nc(-c7ccc8oc9ccccc9c8c7)nc(-c7ccccc7-c7ccccc7)n6)c6ccccc6c5c4c3o2)cc1 ; c1ccc(-c2ccc(-c3nc(-c4cccc5c4ccc4oc6ccc7oc(-c8ccccc8)nc7c6c45)nc(-c4cccc5oc6ccccc6c45)n3)cc2)cc1]

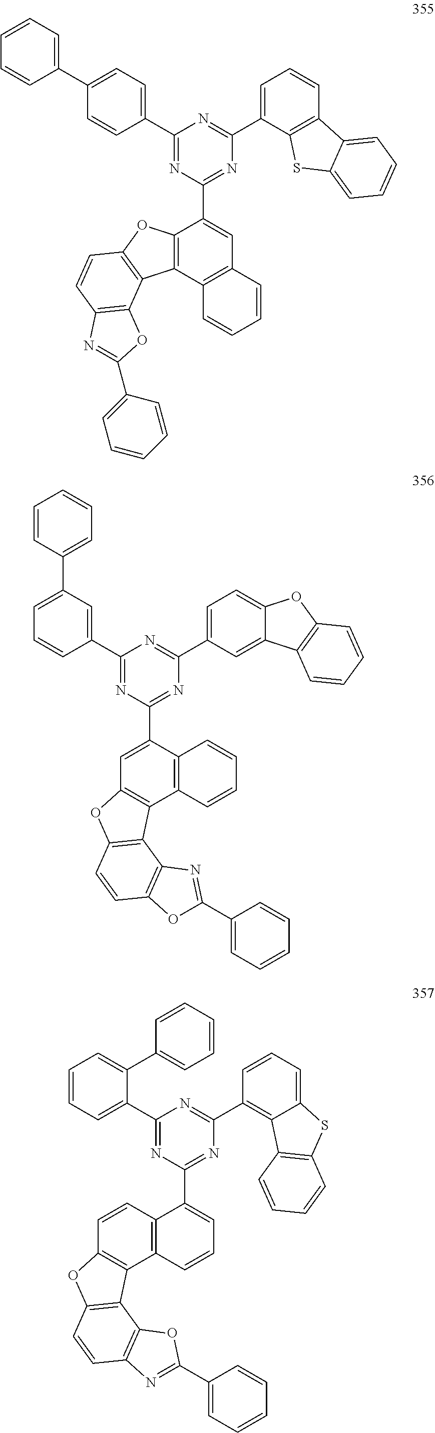 [SMILES: c1ccc(-c2ccc(-c3nc(-c4cc5ccccc5c5c4oc4ccc6nc(-c7ccccc7)oc6c45)nc(-c4cccc5c4sc4ccccc45)n3)cc2)cc1 ; c1ccc(-c2cccc(-c3nc(-c4ccc5oc6ccccc6c5c4)nc(-c4cc5oc6ccc7oc(-c8ccccc8)nc7c6c5c5ccccc45)n3)c2)cc1 ; c1ccc(-c2nc3ccc4oc5ccc6c(-c7nc(-c8ccccc8-c8ccccc8)nc(-c8cccc9sc%10ccccc%10c89)n7)cccc6c5c4c3o2)cc1]

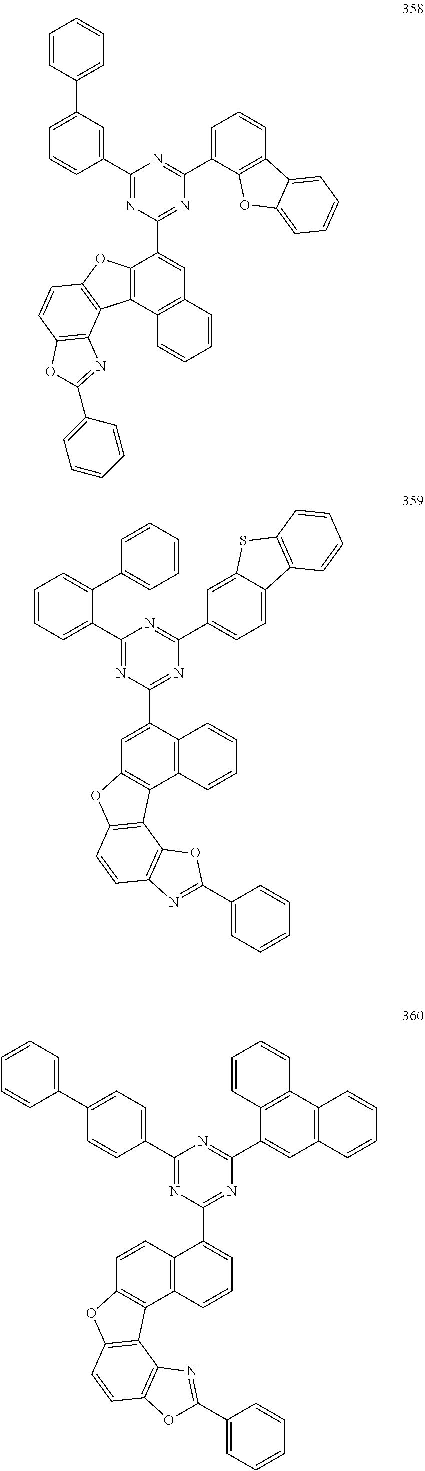 [SMILES: c1ccc(-c2cccc(-c3nc(-c4cccc5c4oc4ccccc45)nc(-c4cc5ccccc5c5c4oc4ccc6oc(-c7ccccc7)nc6c45)n3)c2)cc1 ; c1ccc(-c2nc3ccc4oc5cc(-c6nc(-c7ccc8c(c7)sc7ccccc78)nc(-c7ccccc7-c7ccccc7)n6)c6ccccc6c5c4c3o2)cc1 ; c1ccc(-c2ccc(-c3nc(-c4cc5ccccc5c5ccccc45)nc(-c4cccc5c4ccc4oc6ccc7oc(-c8ccccc8)nc7c6c45)n3)cc2)cc1]

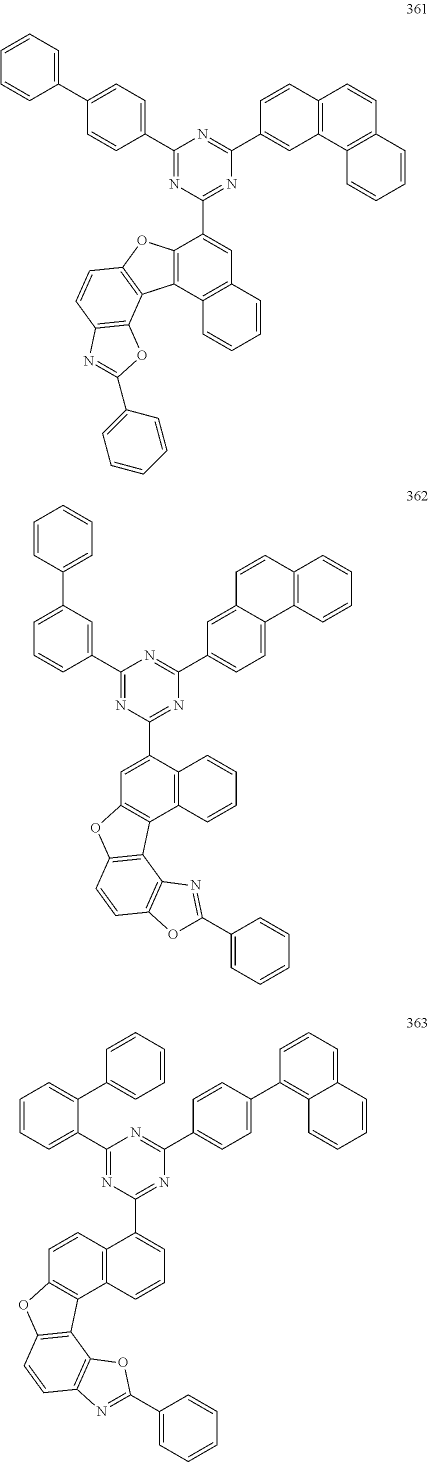 [SMILES: c1ccc(-c2ccc(-c3nc(-c4ccc5ccc6ccccc6c5c4)nc(-c4cc5ccccc5c5c4oc4ccc6nc(-c7ccccc7)oc6c45)n3)cc2)cc1 ; c1ccc(-c2cccc(-c3nc(-c4ccc5c(ccc6ccccc65)c4)nc(-c4cc5oc6ccc7oc(-c8ccccc8)nc7c6c5c5ccccc45)n3)c2)cc1 ; c1ccc(-c2nc3ccc4oc5ccc6c(-c7nc(-c8ccc(-c9cccc%10ccccc9%10)cc8)nc(-c8ccccc8-c8ccccc8)n7)cccc6c5c4c3o2)cc1]

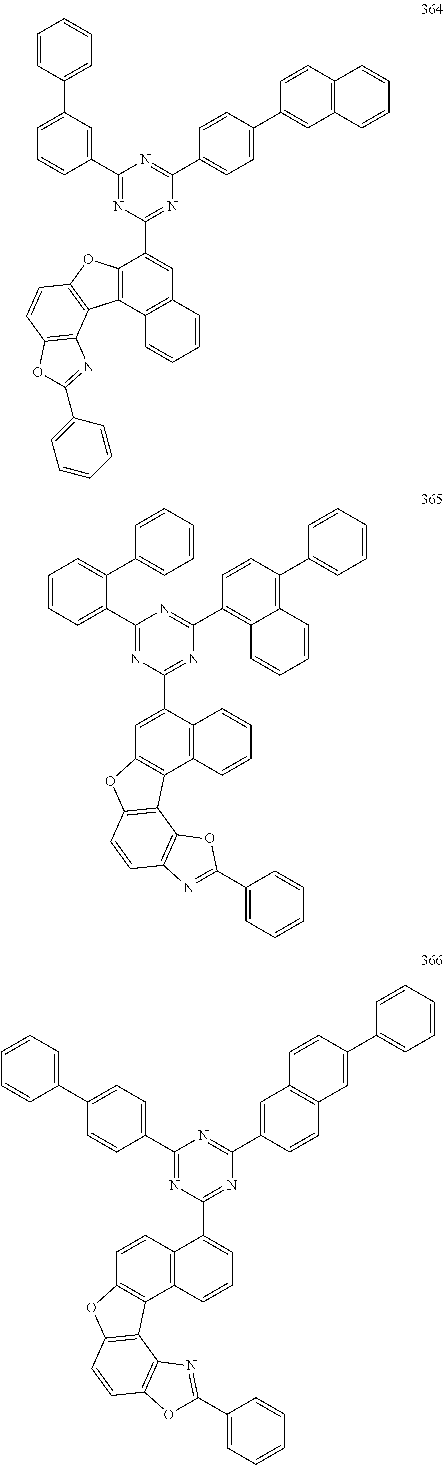 [SMILES: c1ccc(-c2cccc(-c3nc(-c4ccc(-c5ccc6ccccc6c5)cc4)nc(-c4cc5ccccc5c5c4oc4ccc6oc(-c7ccccc7)nc6c45)n3)c2)cc1 ; c1ccc(-c2nc3ccc4oc5cc(-c6nc(-c7ccccc7-c7ccccc7)nc(-c7ccc(-c8ccccc8)c8ccccc78)n6)c6ccccc6c5c4c3o2)cc1 ; c1ccc(-c2ccc(-c3nc(-c4ccc5cc(-c6ccccc6)ccc5c4)nc(-c4cccc5c4ccc4oc6ccc7oc(-c8ccccc8)nc7c6c45)n3)cc2)cc1]

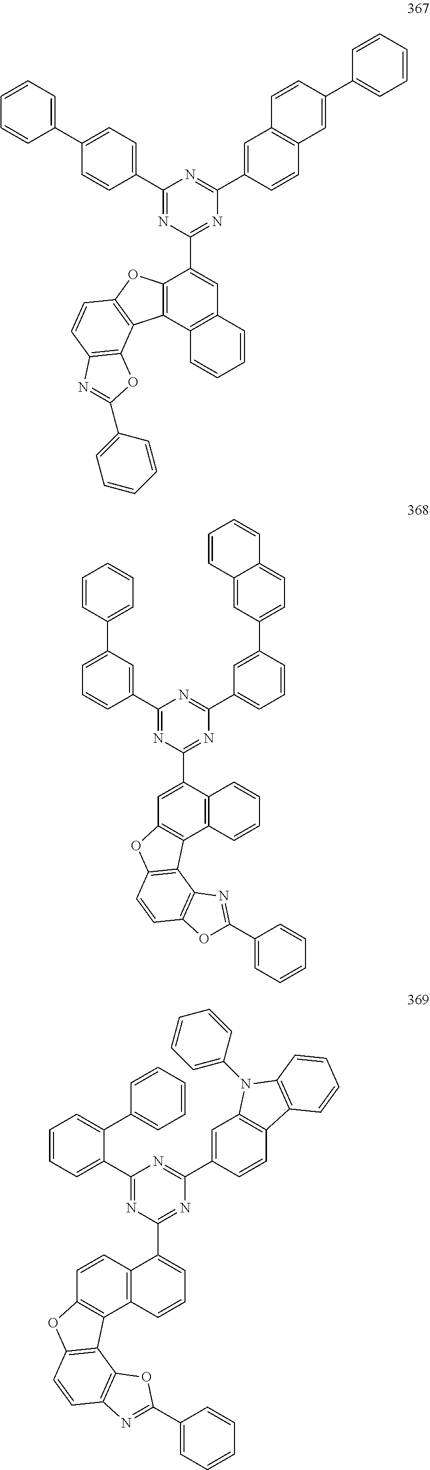 [SMILES: c1ccc(-c2ccc(-c3nc(-c4ccc5cc(-c6ccccc6)ccc5c4)nc(-c4cc5ccccc5c5c4oc4ccc6nc(-c7ccccc7)oc6c45)n3)cc2)cc1 ; c1ccc(-c2cccc(-c3nc(-c4cccc(-c5ccc6ccccc6c5)c4)nc(-c4cc5oc6ccc7oc(-c8ccccc8)nc7c6c5c5ccccc45)n3)c2)cc1 ; c1ccc(-c2nc3ccc4oc5ccc6c(-c7nc(-c8ccc9c%10ccccc%10n(-c%10ccccc%10)c9c8)nc(-c8ccccc8-c8ccccc8)n7)cccc6c5c4c3o2)cc1]

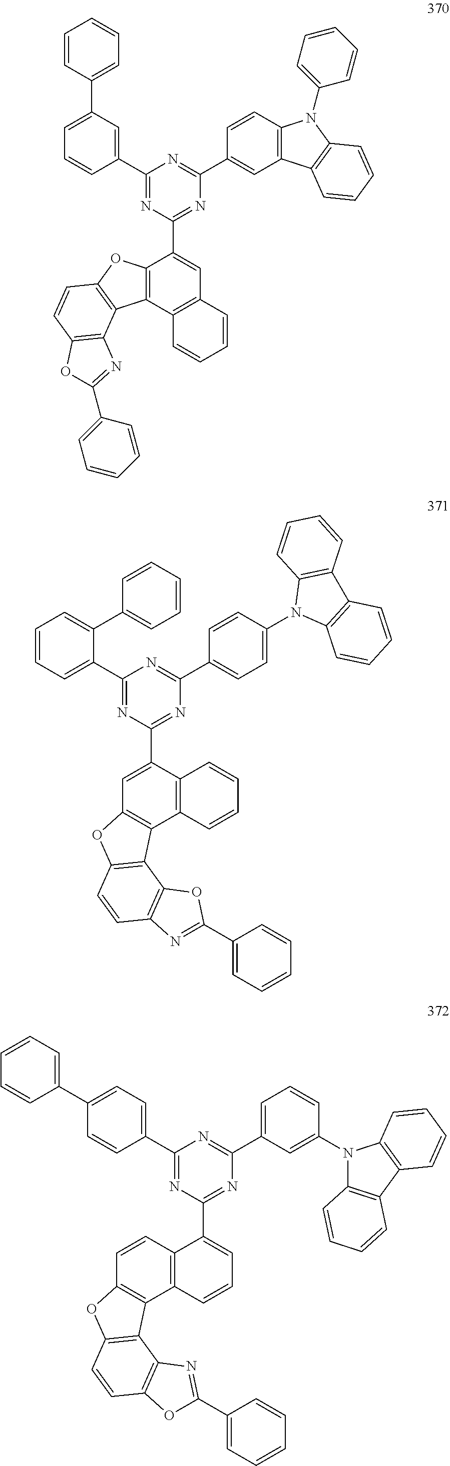 [SMILES: c1ccc(-c2cccc(-c3nc(-c4ccc5c(c4)c4ccccc4n5-c4ccccc4)nc(-c4cc5ccccc5c5c4oc4ccc6oc(-c7ccccc7)nc6c45)n3)c2)cc1 ; c1ccc(-c2nc3ccc4oc5cc(-c6nc(-c7ccc(-n8c9ccccc9c9ccccc98)cc7)nc(-c7ccccc7-c7ccccc7)n6)c6ccccc6c5c4c3o2)cc1 ; c1ccc(-c2ccc(-c3nc(-c4cccc(-n5c6ccccc6c6ccccc65)c4)nc(-c4cccc5c4ccc4oc6ccc7oc(-c8ccccc8)nc7c6c45)n3)cc2)cc1]

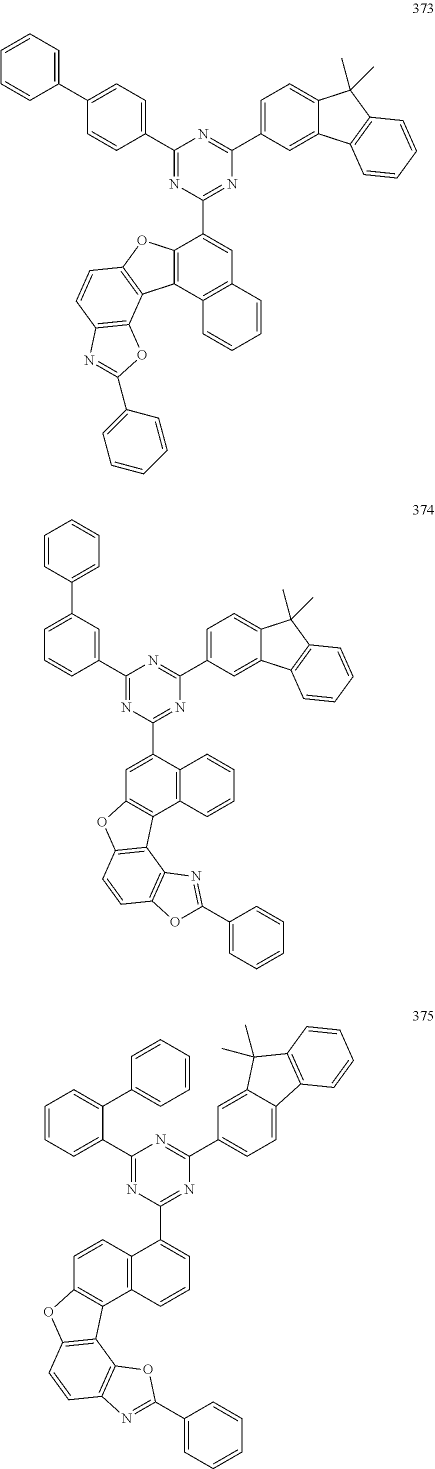 [SMILES: CC1(C)c2ccccc2-c2cc(-c3nc(-c4ccc(-c5ccccc5)cc4)nc(-c4cc5ccccc5c5c4oc4ccc6nc(-c7ccccc7)oc6c45)n3)ccc21 ; CC1(C)c2ccccc2-c2cc(-c3nc(-c4cccc(-c5ccccc5)c4)nc(-c4cc5oc6ccc7oc(-c8ccccc8)nc7c6c5c5ccccc45)n3)ccc21 ; CC1(C)c2ccccc2-c2ccc(-c3nc(-c4ccccc4-c4ccccc4)nc(-c4cccc5c4ccc4oc6ccc7nc(-c8ccccc8)oc7c6c45)n3)cc21]

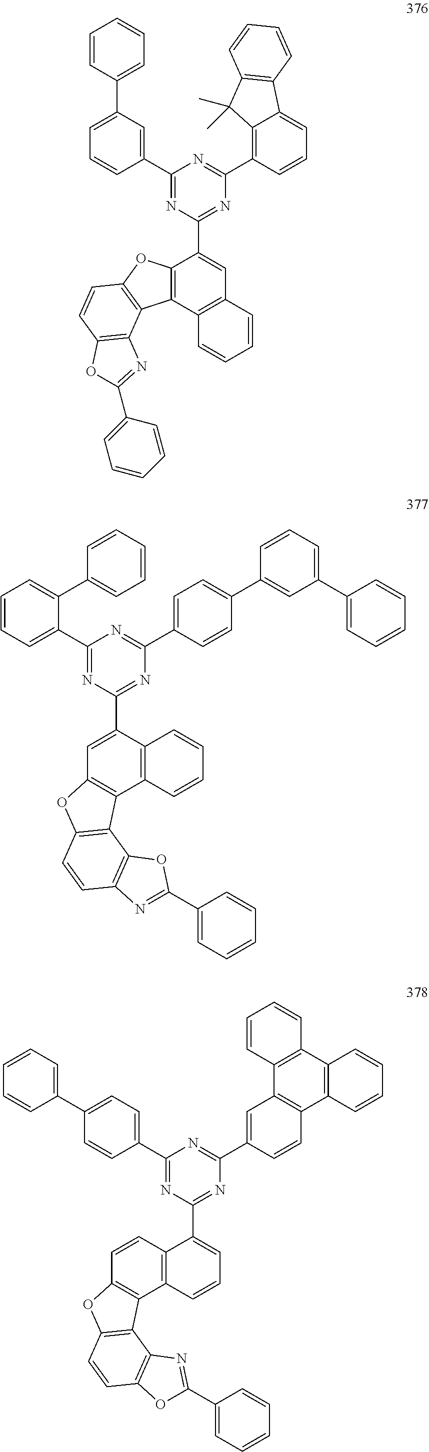 [SMILES: CC1(C)c2ccccc2-c2cccc(-c3nc(-c4cccc(-c5ccccc5)c4)nc(-c4cc5ccccc5c5c4oc4ccc6oc(-c7ccccc7)nc6c45)n3)c21 ; c1ccc(-c2cccc(-c3ccc(-c4nc(-c5ccccc5-c5ccccc5)nc(-c5cc6oc7ccc8nc(-c9ccccc9)oc8c7c6c6ccccc56)n4)cc3)c2)cc1 ; c1ccc(-c2ccc(-c3nc(-c4ccc5c6ccccc6c6ccccc6c5c4)nc(-c4cccc5c4ccc4oc6ccc7oc(-c8ccccc8)nc7c6c45)n3)cc2)cc1]

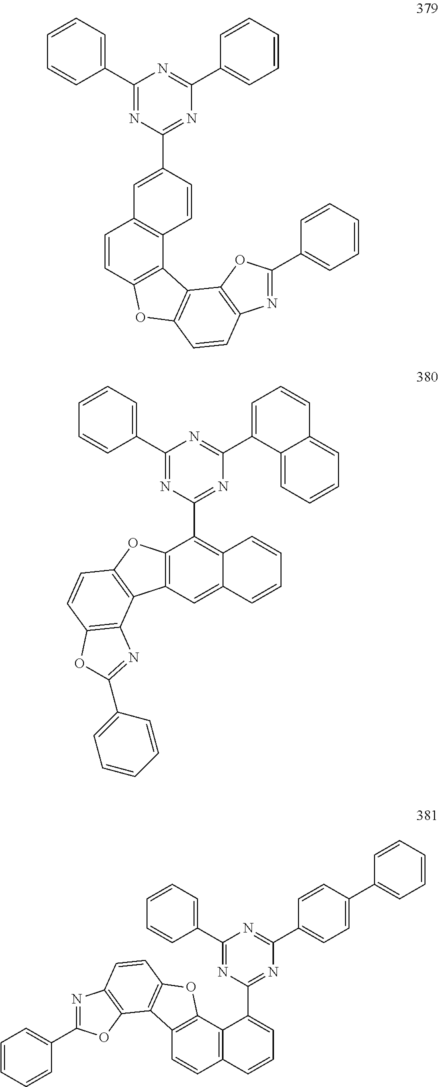 [SMILES: c1ccc(-c2nc(-c3ccccc3)nc(-c3ccc4c(ccc5oc6ccc7nc(-c8ccccc8)oc7c6c54)c3)n2)cc1 ; c1ccc(-c2nc(-c3cccc4ccccc34)nc(-c3c4ccccc4cc4c3oc3ccc5oc(-c6ccccc6)nc5c34)n2)cc1 ; c1ccc(-c2ccc(-c3nc(-c4ccccc4)nc(-c4cccc5ccc6c(oc7ccc8nc(-c9ccccc9)oc8c76)c45)n3)cc2)cc1]

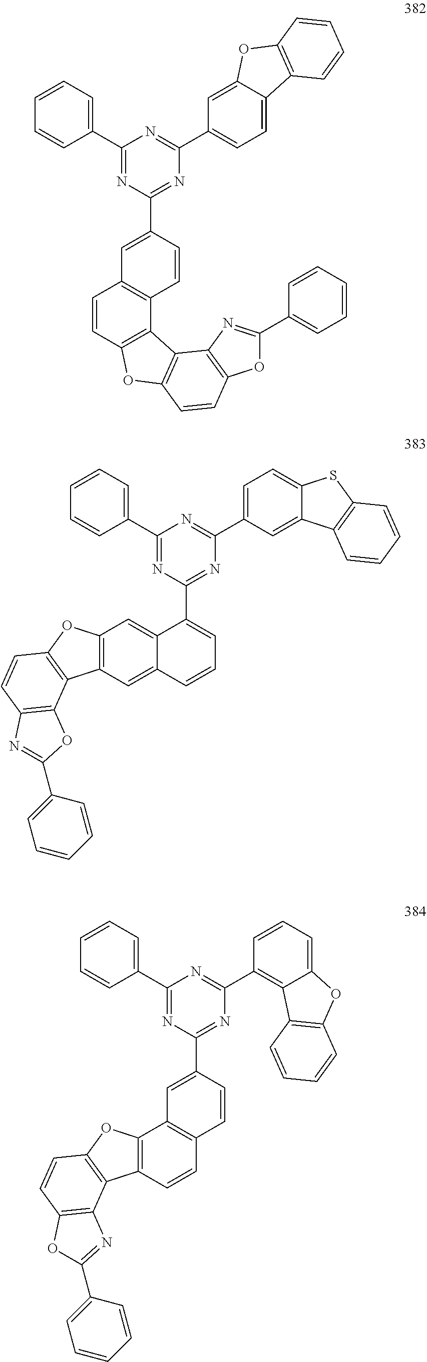 [SMILES: c1ccc(-c2nc(-c3ccc4c(ccc5oc6ccc7oc(-c8ccccc8)nc7c6c54)c3)nc(-c3ccc4c(c3)oc3ccccc34)n2)cc1 ; c1ccc(-c2nc(-c3ccc4sc5ccccc5c4c3)nc(-c3cccc4cc5c(cc34)oc3ccc4nc(-c6ccccc6)oc4c35)n2)cc1 ; c1ccc(-c2nc(-c3ccc4ccc5c(oc6ccc7oc(-c8ccccc8)nc7c65)c4c3)nc(-c3cccc4oc5ccccc5c34)n2)cc1]

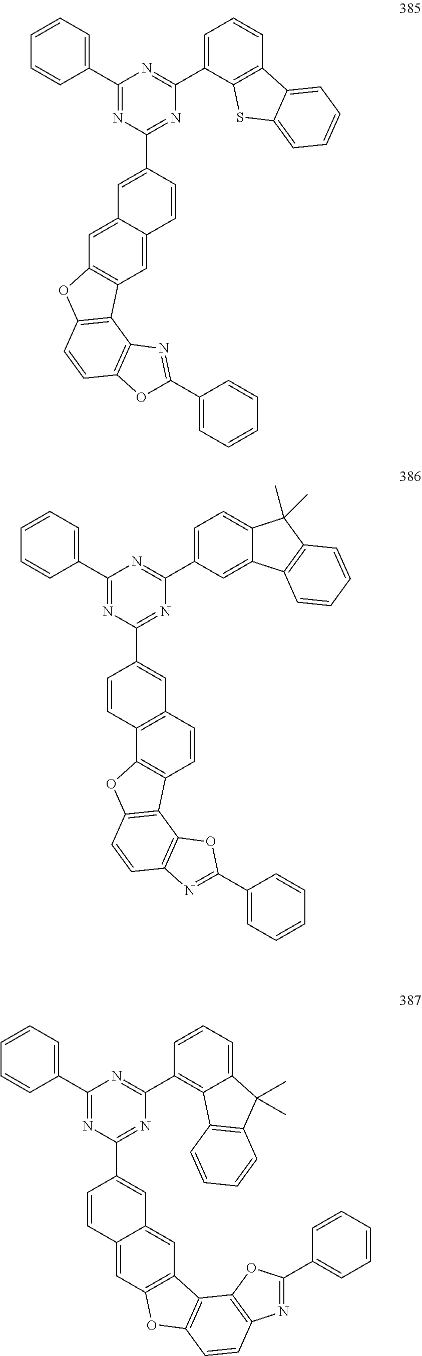 [SMILES: c1ccc(-c2nc(-c3ccc4cc5c(cc4c3)oc3ccc4oc(-c6ccccc6)nc4c35)nc(-c3cccc4c3sc3ccccc34)n2)cc1 ; CC1(C)c2ccccc2-c2cc(-c3nc(-c4ccccc4)nc(-c4ccc5c(ccc6c5oc5ccc7nc(-c8ccccc8)oc7c56)c4)n3)ccc21 ; CC1(C)c2ccccc2-c2c(-c3nc(-c4ccccc4)nc(-c4ccc5cc6oc7ccc8nc(-c9ccccc9)oc8c7c6cc5c4)n3)cccc21]

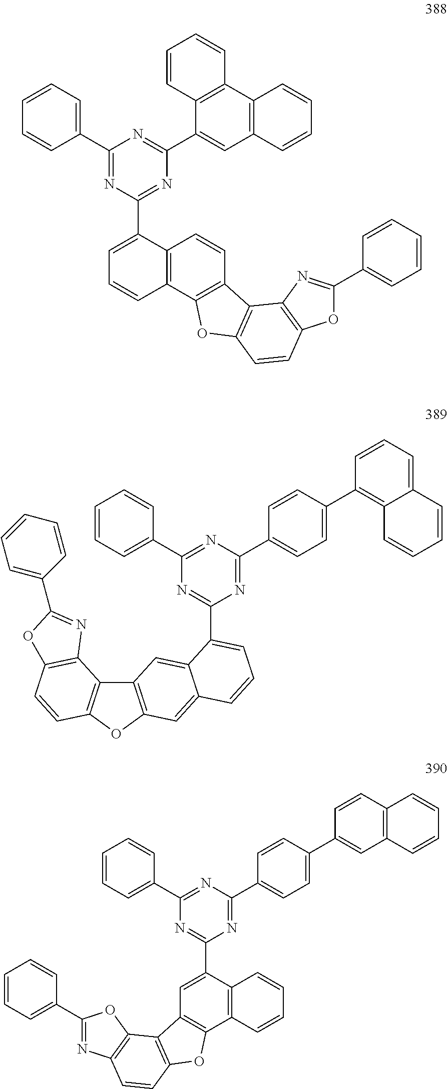 [SMILES: c1ccc(-c2nc(-c3cc4ccccc4c4ccccc34)nc(-c3cccc4c3ccc3c4oc4ccc5oc(-c6ccccc6)nc5c43)n2)cc1 ; c1ccc(-c2nc(-c3ccc(-c4cccc5ccccc45)cc3)nc(-c3cccc4cc5oc6ccc7oc(-c8ccccc8)nc7c6c5cc34)n2)cc1 ; c1ccc(-c2nc(-c3ccc(-c4ccc5ccccc5c4)cc3)nc(-c3cc4c(oc5ccc6nc(-c7ccccc7)oc6c54)c4ccccc34)n2)cc1]

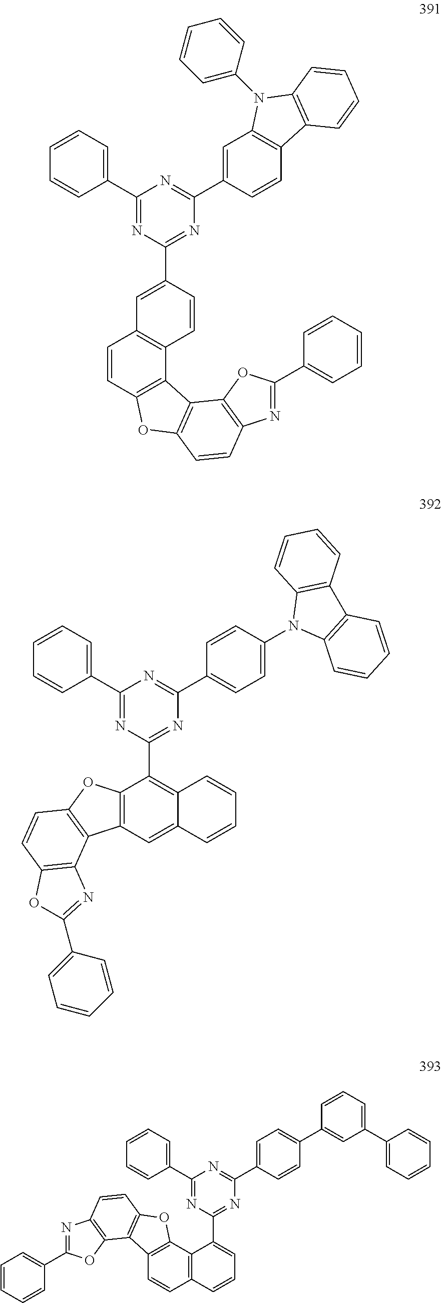 [SMILES: c1ccc(-c2nc(-c3ccc4c(ccc5oc6ccc7nc(-c8ccccc8)oc7c6c54)c3)nc(-c3ccc4c5ccccc5n(-c5ccccc5)c4c3)n2)cc1 ; c1ccc(-c2nc(-c3ccc(-n4c5ccccc5c5ccccc54)cc3)nc(-c3c4ccccc4cc4c3oc3ccc5oc(-c6ccccc6)nc5c34)n2)cc1 ; c1ccc(-c2cccc(-c3ccc(-c4nc(-c5ccccc5)nc(-c5cccc6ccc7c(oc8ccc9nc(-c%10ccccc%10)oc9c87)c56)n4)cc3)c2)cc1]

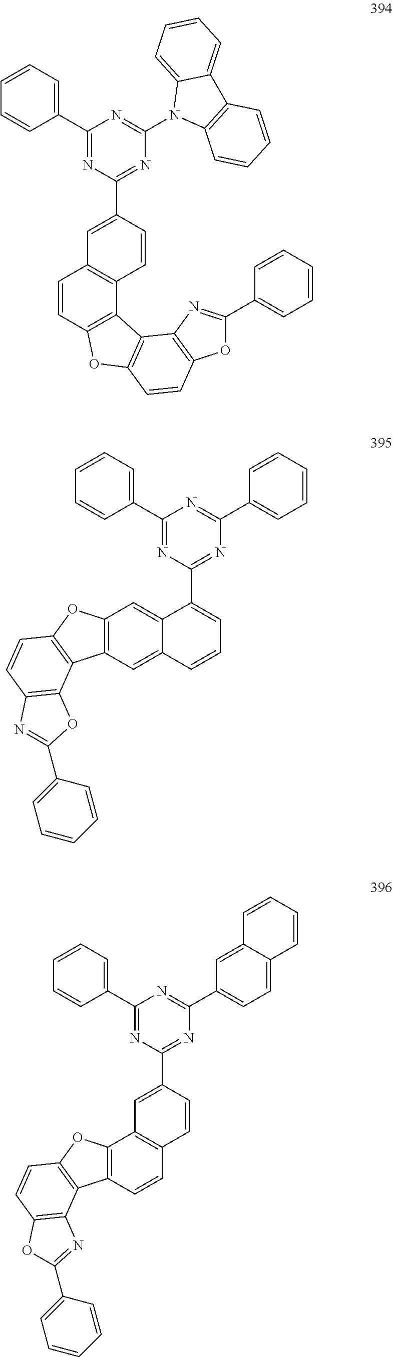 [SMILES: c1ccc(-c2nc(-c3ccc4c(ccc5oc6ccc7oc(-c8ccccc8)nc7c6c54)c3)nc(-n3c4ccccc4c4ccccc43)n2)cc1 ; c1ccc(-c2nc(-c3ccccc3)nc(-c3cccc4cc5c(cc34)oc3ccc4nc(-c6ccccc6)oc4c35)n2)cc1 ; c1ccc(-c2nc(-c3ccc4ccccc4c3)nc(-c3ccc4ccc5c(oc6ccc7oc(-c8ccccc8)nc7c65)c4c3)n2)cc1]

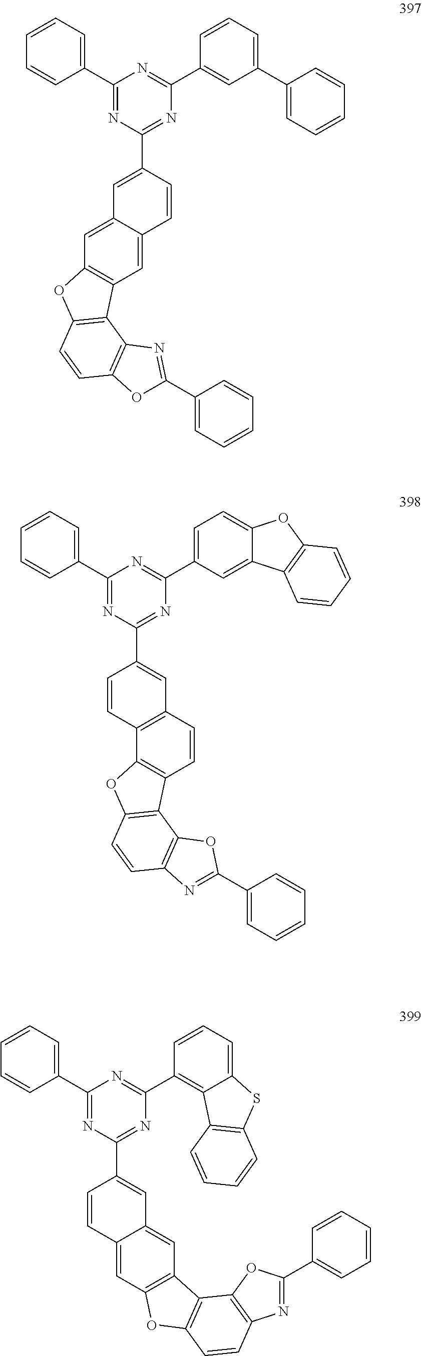 [SMILES: c1ccc(-c2cccc(-c3nc(-c4ccccc4)nc(-c4ccc5cc6c(cc5c4)oc4ccc5oc(-c7ccccc7)nc5c46)n3)c2)cc1 ; c1ccc(-c2nc(-c3ccc4c(ccc5c4oc4ccc6nc(-c7ccccc7)oc6c45)c3)nc(-c3ccc4oc5ccccc5c4c3)n2)cc1 ; c1ccc(-c2nc(-c3ccc4cc5oc6ccc7nc(-c8ccccc8)oc7c6c5cc4c3)nc(-c3cccc4sc5ccccc5c34)n2)cc1]

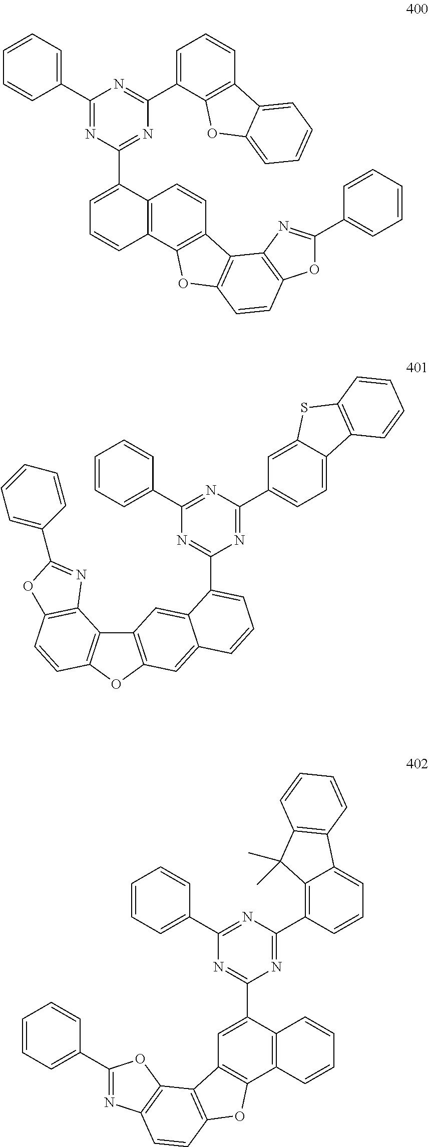 [SMILES: c1ccc(-c2nc(-c3cccc4c3ccc3c4oc4ccc5oc(-c6ccccc6)nc5c43)nc(-c3cccc4c3oc3ccccc34)n2)cc1 ; c1ccc(-c2nc(-c3ccc4c(c3)sc3ccccc34)nc(-c3cccc4cc5oc6ccc7oc(-c8ccccc8)nc7c6c5cc34)n2)cc1 ; CC1(C)c2ccccc2-c2cccc(-c3nc(-c4ccccc4)nc(-c4cc5c(oc6ccc7nc(-c8ccccc8)oc7c65)c5ccccc45)n3)c21]

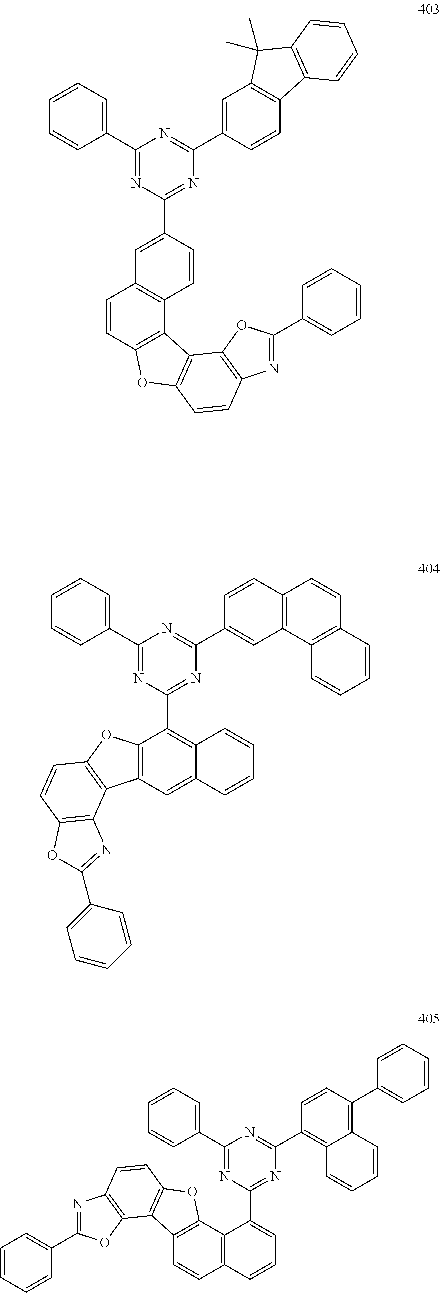 [SMILES: CC1(C)c2ccccc2-c2ccc(-c3nc(-c4ccccc4)nc(-c4ccc5c(ccc6oc7ccc8nc(-c9ccccc9)oc8c7c65)c4)n3)cc21 ; c1ccc(-c2nc(-c3ccc4ccc5ccccc5c4c3)nc(-c3c4ccccc4cc4c3oc3ccc5oc(-c6ccccc6)nc5c34)n2)cc1 ; c1ccc(-c2nc(-c3ccc(-c4ccccc4)c4ccccc34)nc(-c3cccc4ccc5c(oc6ccc7nc(-c8ccccc8)oc7c65)c34)n2)cc1]

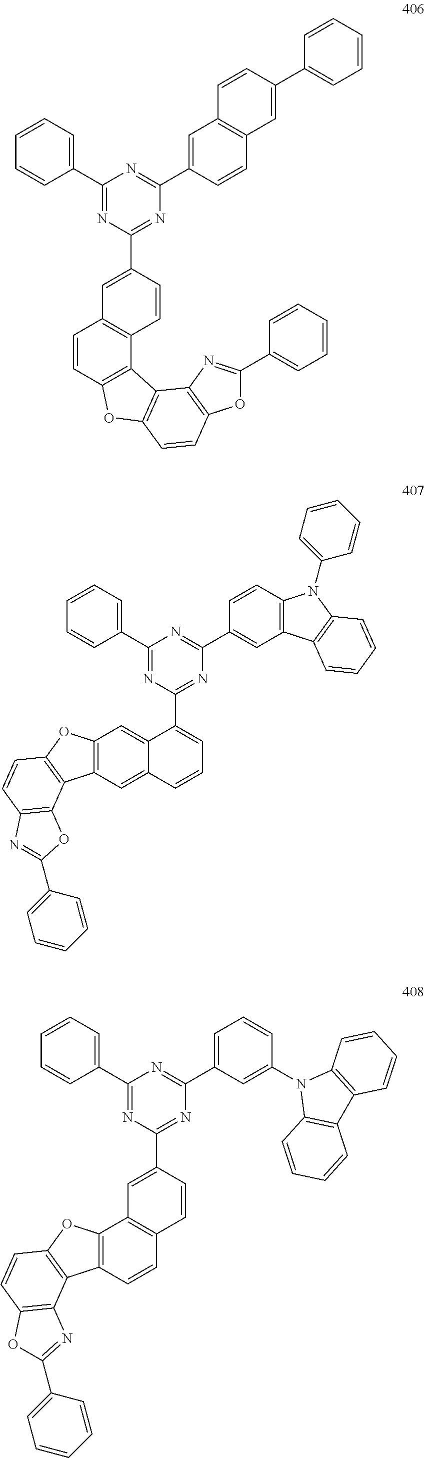 [SMILES: c1ccc(-c2ccc3cc(-c4nc(-c5ccccc5)nc(-c5ccc6c(ccc7oc8ccc9oc(-c%10ccccc%10)nc9c8c76)c5)n4)ccc3c2)cc1 ; c1ccc(-c2nc(-c3ccc4c(c3)c3ccccc3n4-c3ccccc3)nc(-c3cccc4cc5c(cc34)oc3ccc4nc(-c6ccccc6)oc4c35)n2)cc1 ; c1ccc(-c2nc(-c3cccc(-n4c5ccccc5c5ccccc54)c3)nc(-c3ccc4ccc5c(oc6ccc7oc(-c8ccccc8)nc7c65)c4c3)n2)cc1]

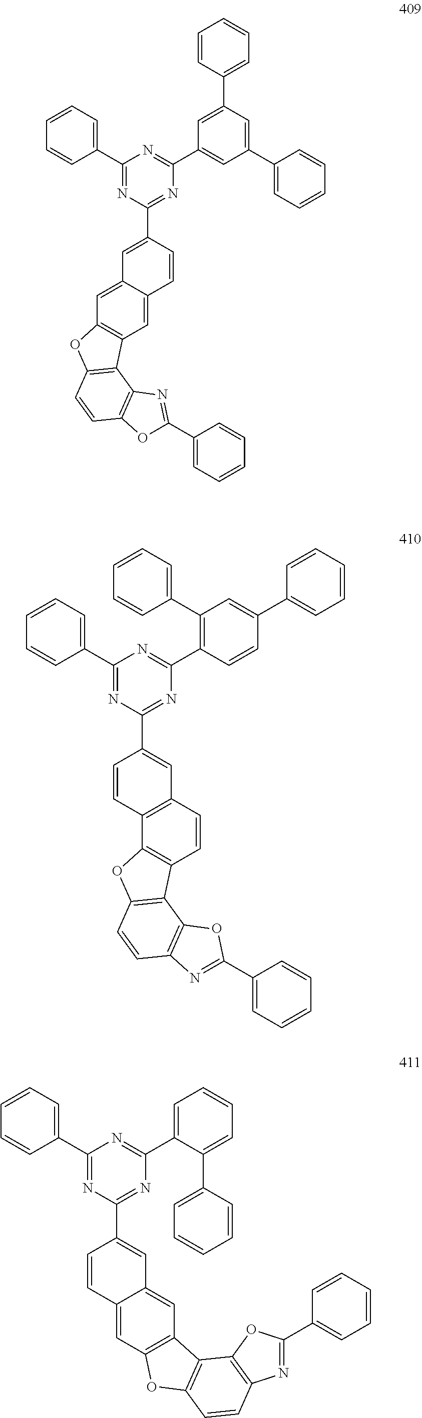 [SMILES: c1ccc(-c2cc(-c3ccccc3)cc(-c3nc(-c4ccccc4)nc(-c4ccc5cc6c(cc5c4)oc4ccc5oc(-c7ccccc7)nc5c46)n3)c2)cc1 ; c1ccc(-c2ccc(-c3nc(-c4ccccc4)nc(-c4ccc5c(ccc6c5oc5ccc7nc(-c8ccccc8)oc7c56)c4)n3)c(-c3ccccc3)c2)cc1 ; c1ccc(-c2nc(-c3ccc4cc5oc6ccc7nc(-c8ccccc8)oc7c6c5cc4c3)nc(-c3ccccc3-c3ccccc3)n2)cc1]

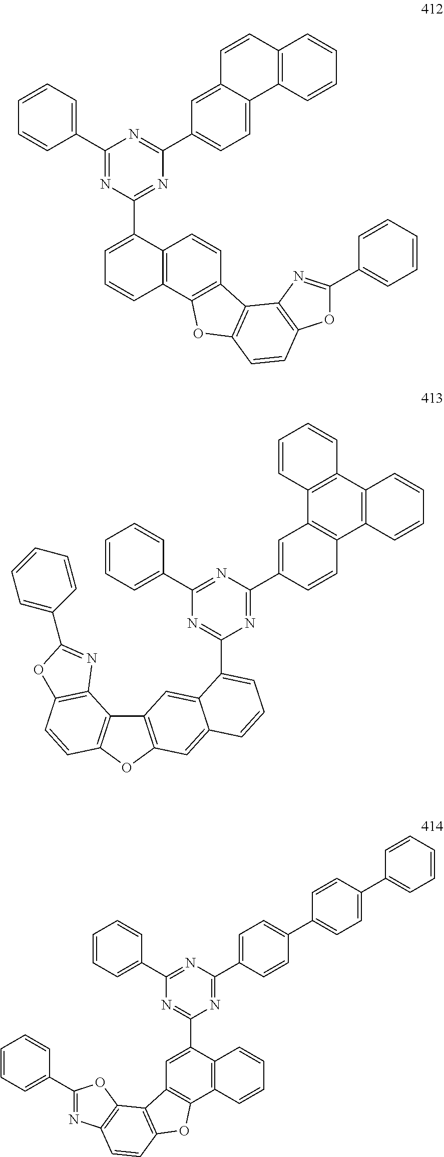 [SMILES: c1ccc(-c2nc(-c3ccc4c(ccc5ccccc54)c3)nc(-c3cccc4c3ccc3c4oc4ccc5oc(-c6ccccc6)nc5c43)n2)cc1 ; c1ccc(-c2nc(-c3ccc4c5ccccc5c5ccccc5c4c3)nc(-c3cccc4cc5oc6ccc7oc(-c8ccccc8)nc7c6c5cc34)n2)cc1 ; c1ccc(-c2ccc(-c3ccc(-c4nc(-c5ccccc5)nc(-c5cc6c(oc7ccc8nc(-c9ccccc9)oc8c76)c6ccccc56)n4)cc3)cc2)cc1]

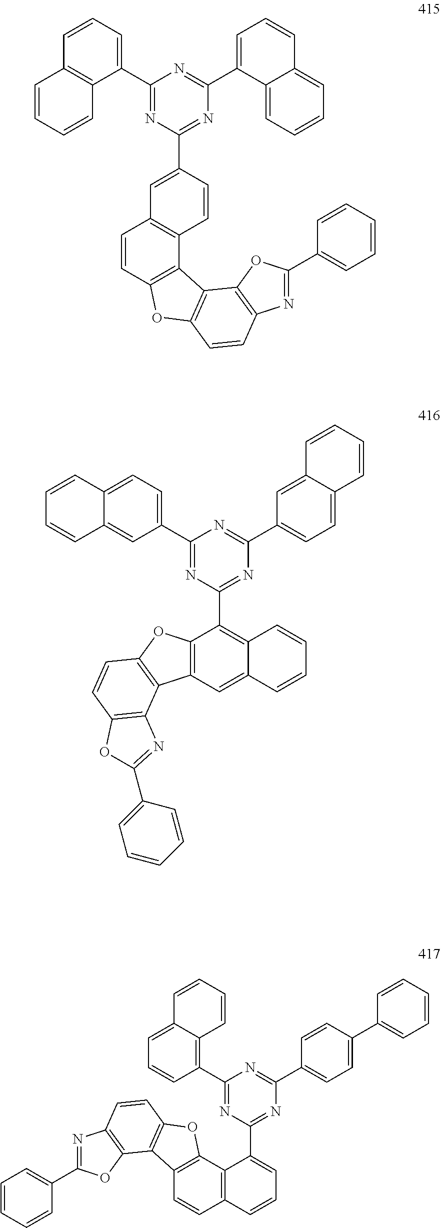 [SMILES: c1ccc(-c2nc3ccc4oc5ccc6cc(-c7nc(-c8cccc9ccccc89)nc(-c8cccc9ccccc89)n7)ccc6c5c4c3o2)cc1 ; c1ccc(-c2nc3c(ccc4oc5c(-c6nc(-c7ccc8ccccc8c7)nc(-c7ccc8ccccc8c7)n6)c6ccccc6cc5c43)o2)cc1 ; c1ccc(-c2ccc(-c3nc(-c4cccc5ccccc45)nc(-c4cccc5ccc6c(oc7ccc8nc(-c9ccccc9)oc8c76)c45)n3)cc2)cc1]

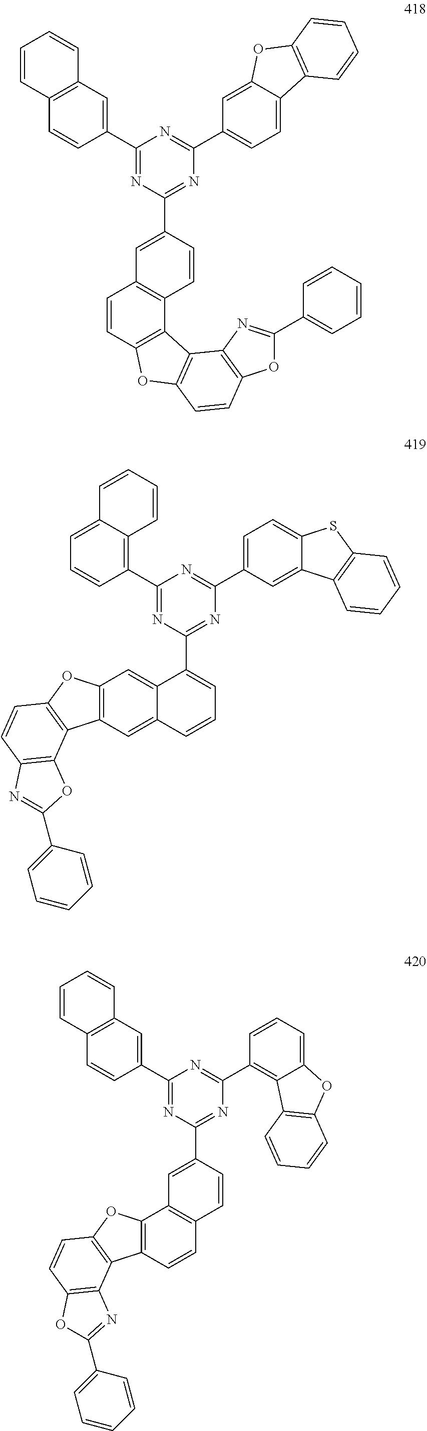 [SMILES: c1ccc(-c2nc3c(ccc4oc5ccc6cc(-c7nc(-c8ccc9ccccc9c8)nc(-c8ccc9c(c8)oc8ccccc89)n7)ccc6c5c43)o2)cc1 ; c1ccc(-c2nc3ccc4oc5cc6c(-c7nc(-c8ccc9sc%10ccccc%10c9c8)nc(-c8cccc9ccccc89)n7)cccc6cc5c4c3o2)cc1 ; c1ccc(-c2nc3c(ccc4oc5c6cc(-c7nc(-c8ccc9ccccc9c8)nc(-c8cccc9oc%10ccccc%10c89)n7)ccc6ccc5c43)o2)cc1]

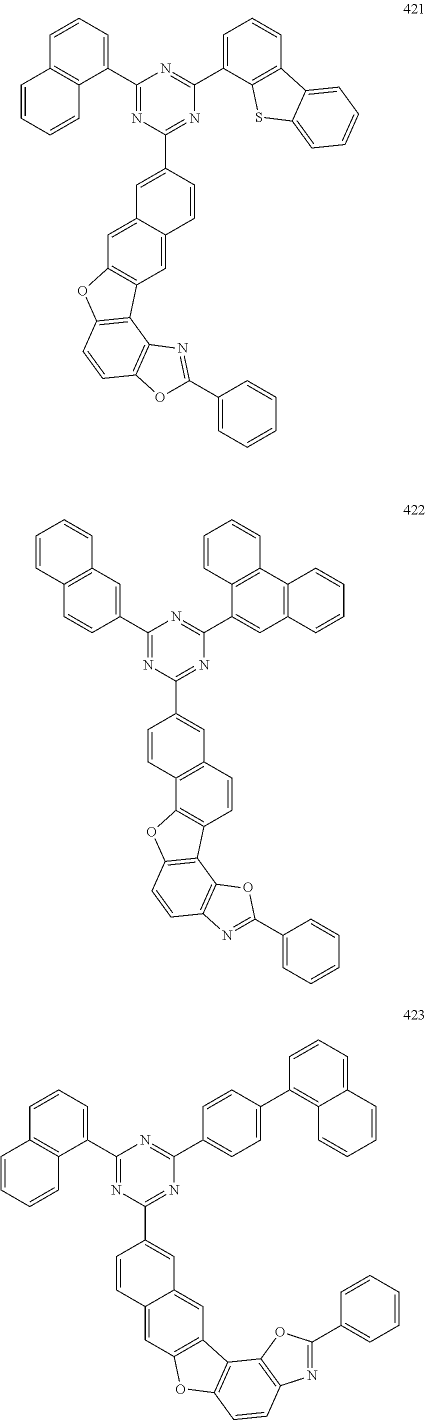 [SMILES: c1ccc(-c2nc3c(ccc4oc5cc6cc(-c7nc(-c8cccc9ccccc89)nc(-c8cccc9c8sc8ccccc89)n7)ccc6cc5c43)o2)cc1 ; c1ccc(-c2nc3ccc4oc5c6ccc(-c7nc(-c8ccc9ccccc9c8)nc(-c8cc9ccccc9c9ccccc89)n7)cc6ccc5c4c3o2)cc1 ; c1ccc(-c2nc3ccc4oc5cc6ccc(-c7nc(-c8ccc(-c9cccc%10ccccc9%10)cc8)nc(-c8cccc9ccccc89)n7)cc6cc5c4c3o2)cc1]

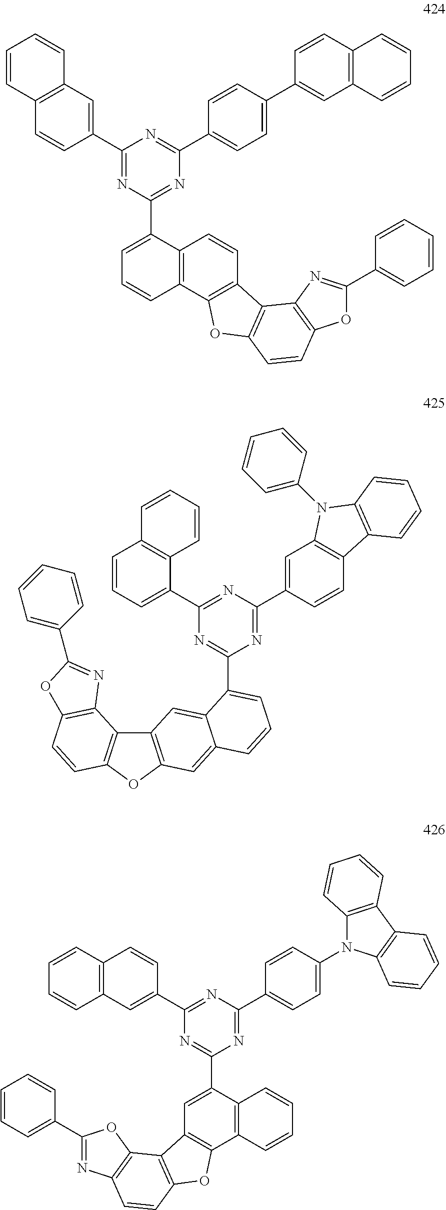 [SMILES: c1ccc(-c2nc3c(ccc4oc5c6cccc(-c7nc(-c8ccc(-c9ccc%10ccccc%10c9)cc8)nc(-c8ccc9ccccc9c8)n7)c6ccc5c43)o2)cc1 ; c1ccc(-c2nc3c(ccc4oc5cc6cccc(-c7nc(-c8ccc9c%10ccccc%10n(-c%10ccccc%10)c9c8)nc(-c8cccc9ccccc89)n7)c6cc5c43)o2)cc1 ; c1ccc(-c2nc3ccc4oc5c6ccccc6c(-c6nc(-c7ccc(-n8c9ccccc9c9ccccc98)cc7)nc(-c7ccc8ccccc8c7)n6)cc5c4c3o2)cc1]

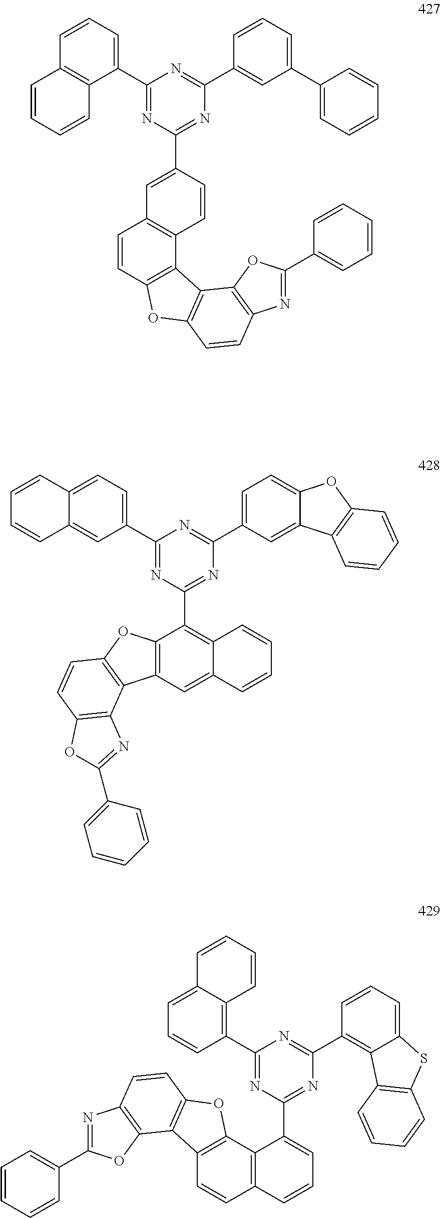 [SMILES: c1ccc(-c2cccc(-c3nc(-c4ccc5c(ccc6oc7ccc8nc(-c9ccccc9)oc8c7c65)c4)nc(-c4cccc5ccccc45)n3)c2)cc1 ; c1ccc(-c2nc3c(ccc4oc5c(-c6nc(-c7ccc8ccccc8c7)nc(-c7ccc8oc9ccccc9c8c7)n6)c6ccccc6cc5c43)o2)cc1 ; c1ccc(-c2nc3ccc4oc5c(ccc6cccc(-c7nc(-c8cccc9ccccc89)nc(-c8cccc9sc%10ccccc%10c89)n7)c65)c4c3o2)cc1]

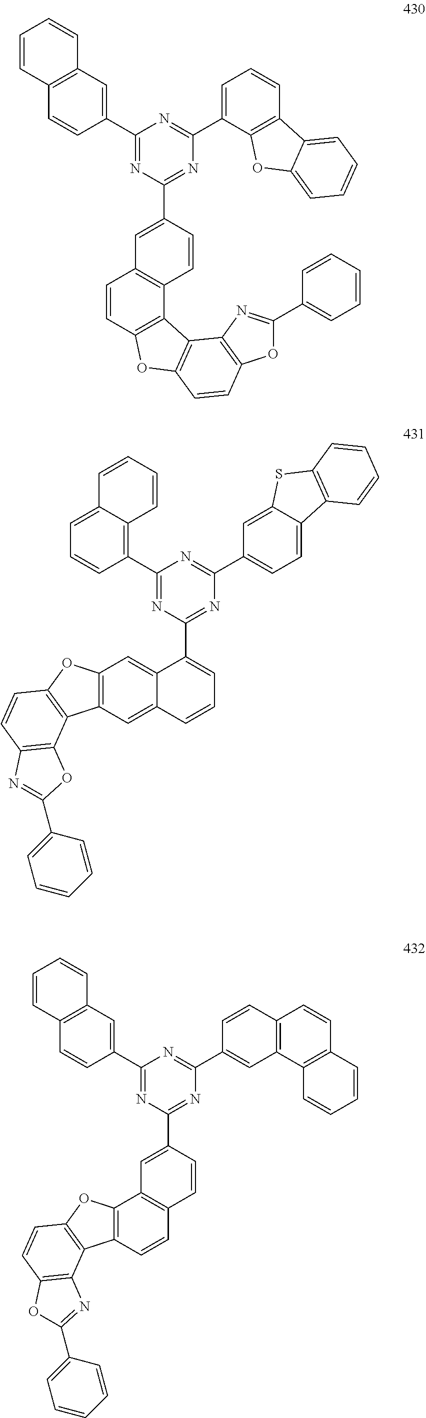 [SMILES: c1ccc(-c2nc3c(ccc4oc5ccc6cc(-c7nc(-c8ccc9ccccc9c8)nc(-c8cccc9c8oc8ccccc89)n7)ccc6c5c43)o2)cc1 ; c1ccc(-c2nc3ccc4oc5cc6c(-c7nc(-c8ccc9c(c8)sc8ccccc89)nc(-c8cccc9ccccc89)n7)cccc6cc5c4c3o2)cc1 ; c1ccc(-c2nc3c(ccc4oc5c6cc(-c7nc(-c8ccc9ccccc9c8)nc(-c8ccc9ccc%10ccccc%10c9c8)n7)ccc6ccc5c43)o2)cc1]

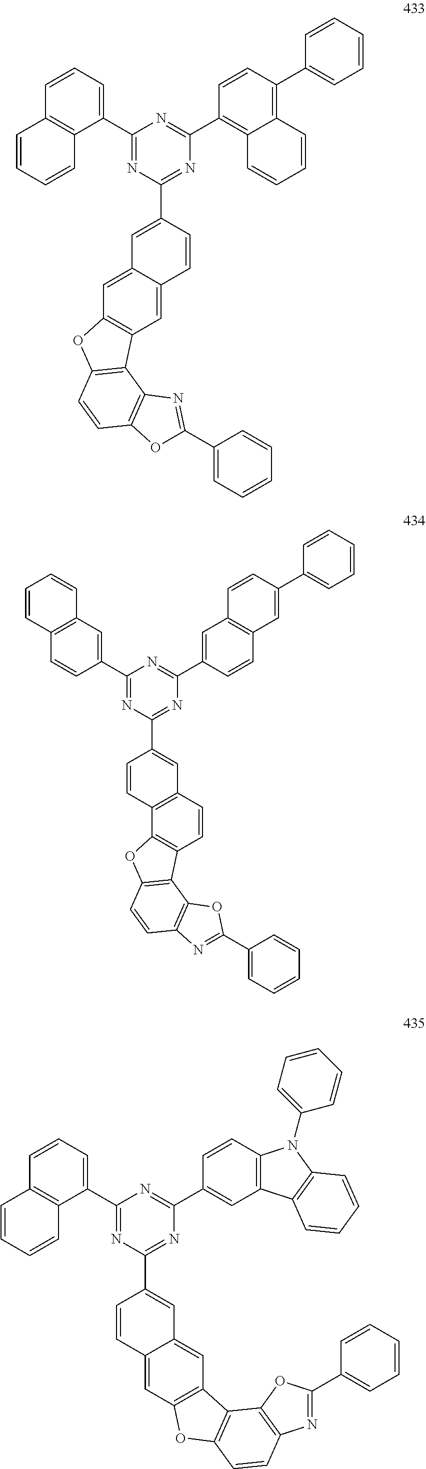 [SMILES: c1ccc(-c2nc3c(ccc4oc5cc6cc(-c7nc(-c8cccc9ccccc89)nc(-c8ccc(-c9ccccc9)c9ccccc89)n7)ccc6cc5c43)o2)cc1 ; c1ccc(-c2ccc3cc(-c4nc(-c5ccc6ccccc6c5)nc(-c5ccc6c(ccc7c6oc6ccc8nc(-c9ccccc9)oc8c67)c5)n4)ccc3c2)cc1 ; c1ccc(-c2nc3ccc4oc5cc6ccc(-c7nc(-c8ccc9c(c8)c8ccccc8n9-c8ccccc8)nc(-c8cccc9ccccc89)n7)cc6cc5c4c3o2)cc1]

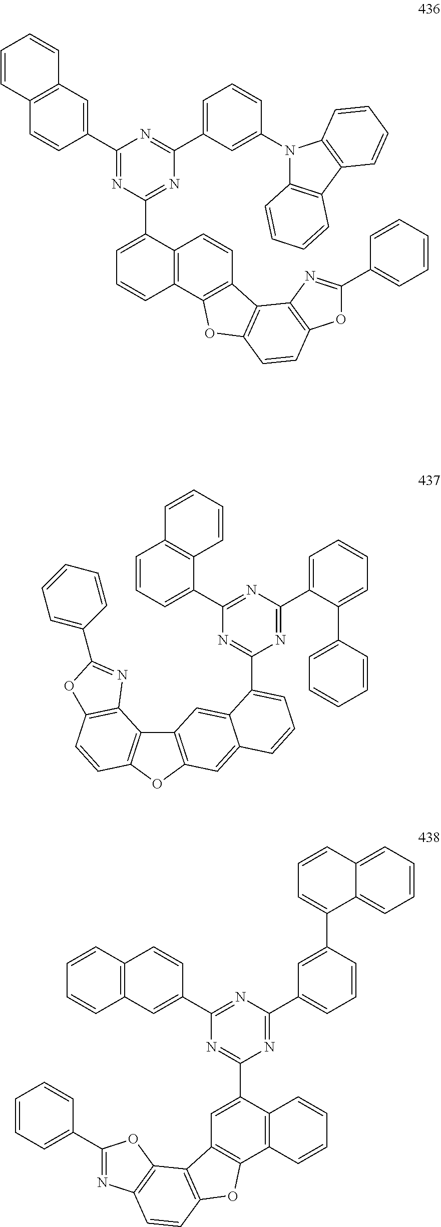 [SMILES: c1ccc(-c2nc3c(ccc4oc5c6cccc(-c7nc(-c8cccc(-n9c%10ccccc%10c%10ccccc%109)c8)nc(-c8ccc9ccccc9c8)n7)c6ccc5c43)o2)cc1 ; c1ccc(-c2nc3c(ccc4oc5cc6cccc(-c7nc(-c8ccccc8-c8ccccc8)nc(-c8cccc9ccccc89)n7)c6cc5c43)o2)cc1 ; c1ccc(-c2nc3ccc4oc5c6ccccc6c(-c6nc(-c7cccc(-c8cccc9ccccc89)c7)nc(-c7ccc8ccccc8c7)n6)cc5c4c3o2)cc1]

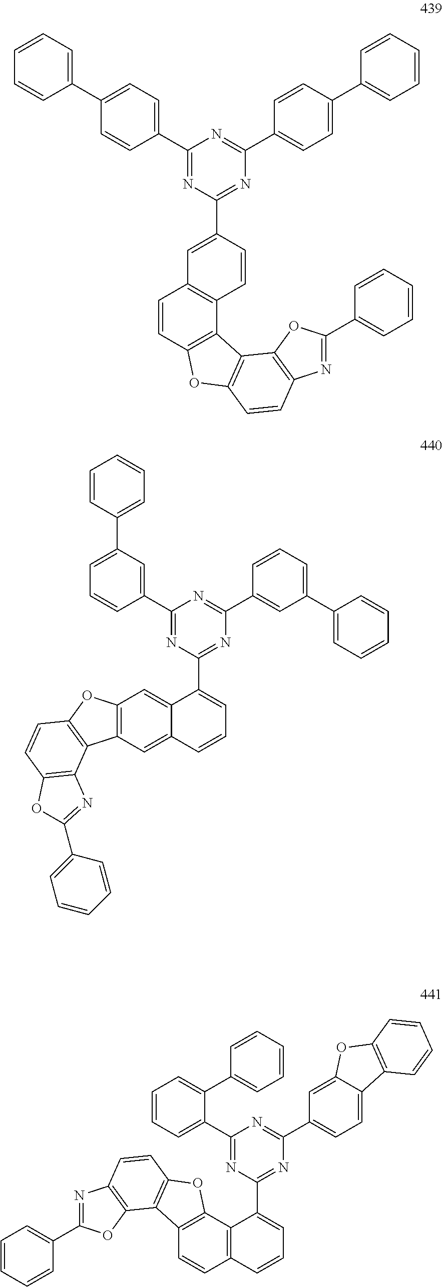 [SMILES: c1ccc(-c2ccc(-c3nc(-c4ccc(-c5ccccc5)cc4)nc(-c4ccc5c(ccc6oc7ccc8nc(-c9ccccc9)oc8c7c65)c4)n3)cc2)cc1 ; c1ccc(-c2cccc(-c3nc(-c4cccc(-c5ccccc5)c4)nc(-c4cccc5cc6c(cc45)oc4ccc5oc(-c7ccccc7)nc5c46)n3)c2)cc1 ; c1ccc(-c2nc3ccc4oc5c(ccc6cccc(-c7nc(-c8ccc9c(c8)oc8ccccc89)nc(-c8ccccc8-c8ccccc8)n7)c65)c4c3o2)cc1]

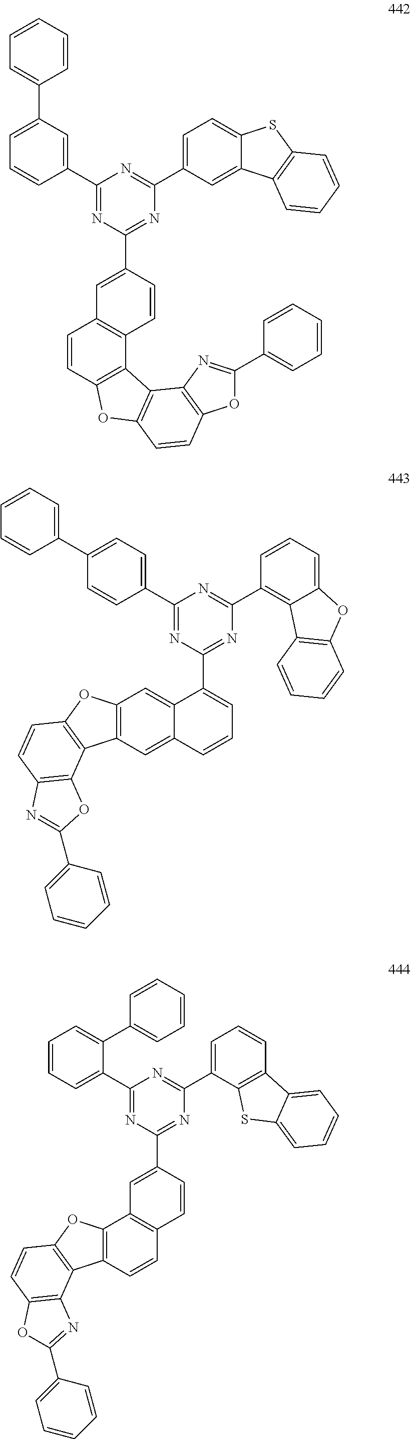 [SMILES: c1ccc(-c2cccc(-c3nc(-c4ccc5c(ccc6oc7ccc8oc(-c9ccccc9)nc8c7c65)c4)nc(-c4ccc5sc6ccccc6c5c4)n3)c2)cc1 ; c1ccc(-c2ccc(-c3nc(-c4cccc5cc6c(cc45)oc4ccc5nc(-c7ccccc7)oc5c46)nc(-c4cccc5oc6ccccc6c45)n3)cc2)cc1 ; c1ccc(-c2nc3c(ccc4oc5c6cc(-c7nc(-c8ccccc8-c8ccccc8)nc(-c8cccc9c8sc8ccccc89)n7)ccc6ccc5c43)o2)cc1]

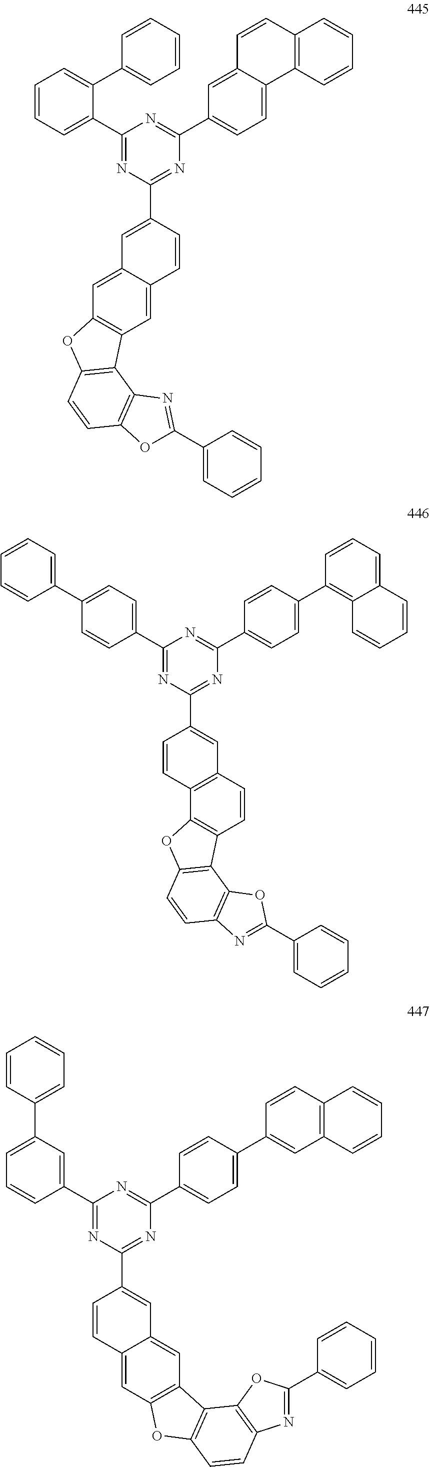 [SMILES: c1ccc(-c2nc3c(ccc4oc5cc6cc(-c7nc(-c8ccc9c(ccc%10ccccc%109)c8)nc(-c8ccccc8-c8ccccc8)n7)ccc6cc5c43)o2)cc1 ; c1ccc(-c2ccc(-c3nc(-c4ccc(-c5cccc6ccccc56)cc4)nc(-c4ccc5c(ccc6c5oc5ccc7nc(-c8ccccc8)oc7c56)c4)n3)cc2)cc1 ; c1ccc(-c2cccc(-c3nc(-c4ccc(-c5ccc6ccccc6c5)cc4)nc(-c4ccc5cc6oc7ccc8nc(-c9ccccc9)oc8c7c6cc5c4)n3)c2)cc1]

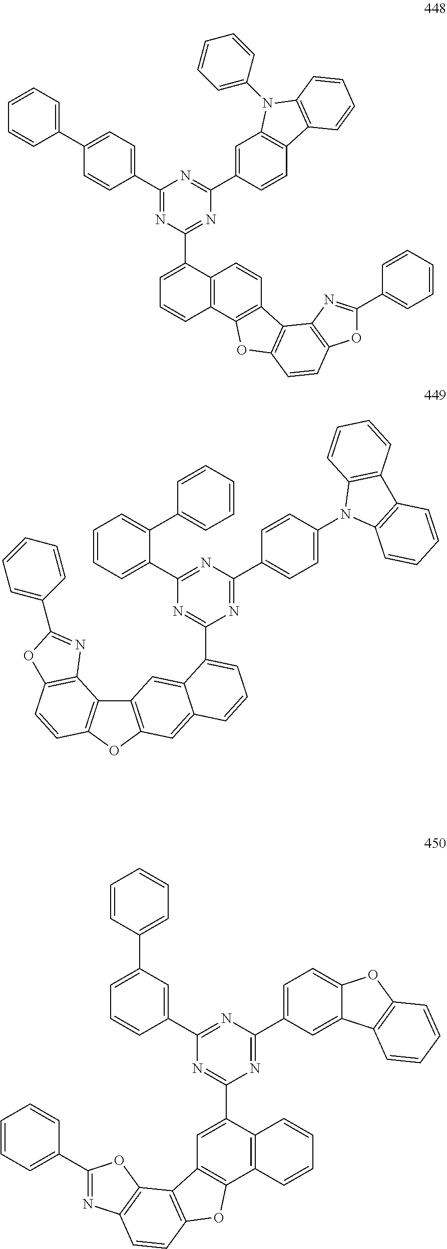 [SMILES: c1ccc(-c2ccc(-c3nc(-c4ccc5c6ccccc6n(-c6ccccc6)c5c4)nc(-c4cccc5c4ccc4c5oc5ccc6oc(-c7ccccc7)nc6c54)n3)cc2)cc1 ; c1ccc(-c2nc3c(ccc4oc5cc6cccc(-c7nc(-c8ccc(-n9c%10ccccc%10c%10ccccc%109)cc8)nc(-c8ccccc8-c8ccccc8)n7)c6cc5c43)o2)cc1 ; c1ccc(-c2cccc(-c3nc(-c4ccc5oc6ccccc6c5c4)nc(-c4cc5c(oc6ccc7nc(-c8ccccc8)oc7c65)c5ccccc45)n3)c2)cc1]

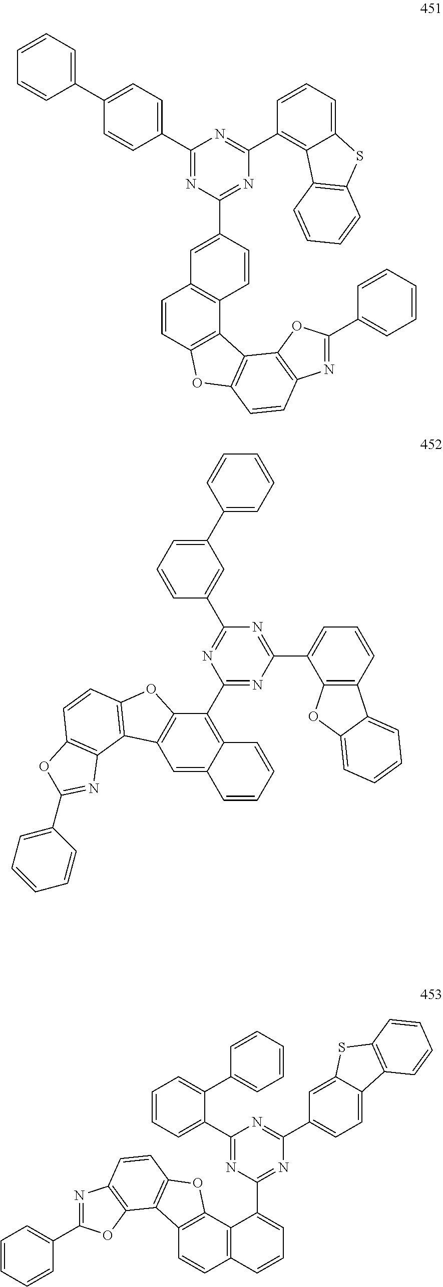

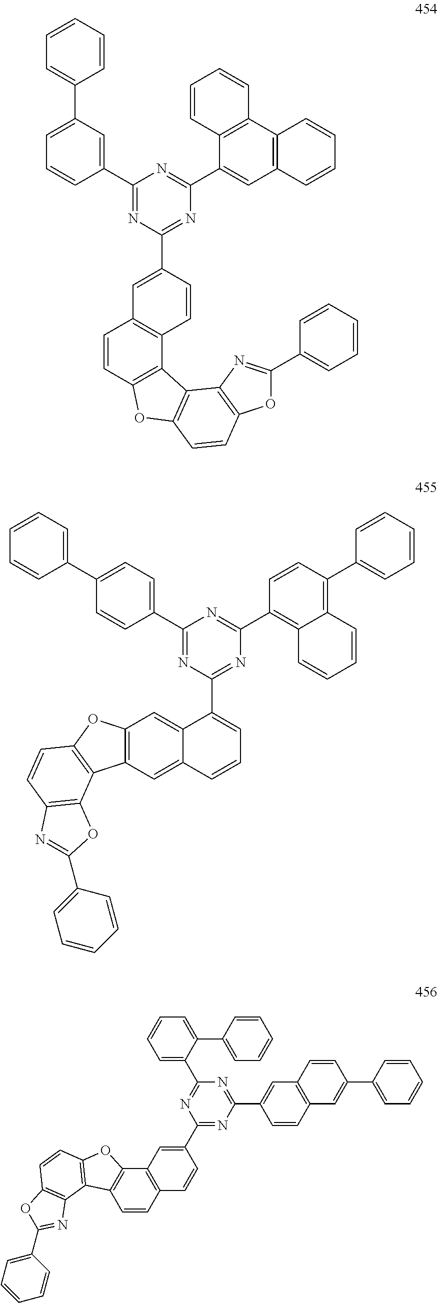 [SMILES: c1ccc(-c2cccc(-c3nc(-c4ccc5c(ccc6oc7ccc8oc(-c9ccccc9)nc8c7c65)c4)nc(-c4cc5ccccc5c5ccccc45)n3)c2)cc1 ; c1ccc(-c2ccc(-c3nc(-c4cccc5cc6c(cc45)oc4ccc5nc(-c7ccccc7)oc5c46)nc(-c4ccc(-c5ccccc5)c5ccccc45)n3)cc2)cc1 ; c1ccc(-c2ccc3cc(-c4nc(-c5ccc6ccc7c(oc8ccc9oc(-c%10ccccc%10)nc9c87)c6c5)nc(-c5ccccc5-c5ccccc5)n4)ccc3c2)cc1]

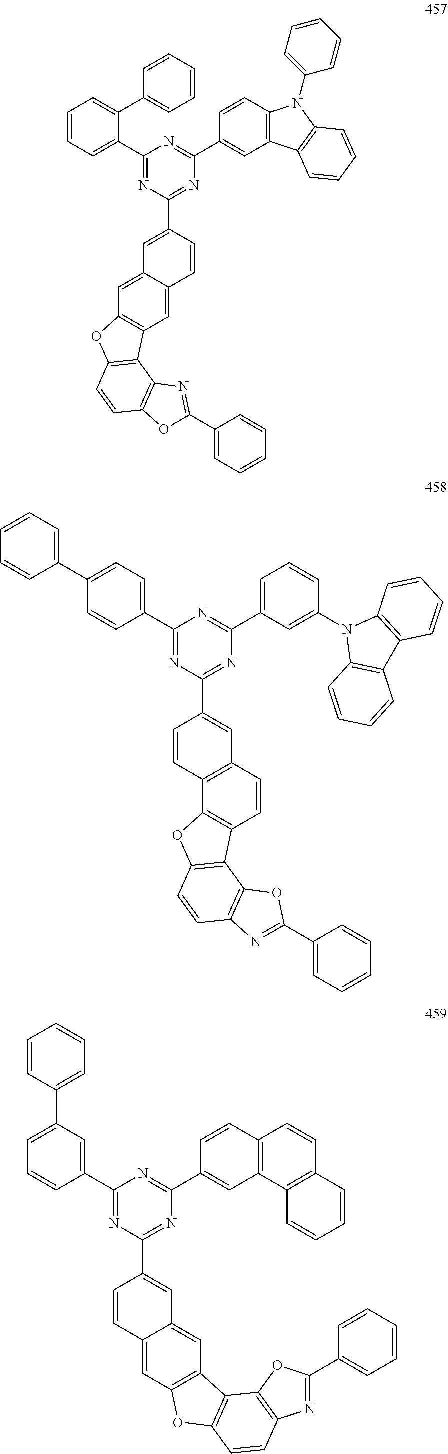 [SMILES: c1ccc(-c2nc3c(ccc4oc5cc6cc(-c7nc(-c8ccc9c(c8)c8ccccc8n9-c8ccccc8)nc(-c8ccccc8-c8ccccc8)n7)ccc6cc5c43)o2)cc1 ; c1ccc(-c2ccc(-c3nc(-c4cccc(-n5c6ccccc6c6ccccc65)c4)nc(-c4ccc5c(ccc6c5oc5ccc7nc(-c8ccccc8)oc7c56)c4)n3)cc2)cc1 ; c1ccc(-c2cccc(-c3nc(-c4ccc5cc6oc7ccc8nc(-c9ccccc9)oc8c7c6cc5c4)nc(-c4ccc5ccc6ccccc6c5c4)n3)c2)cc1]

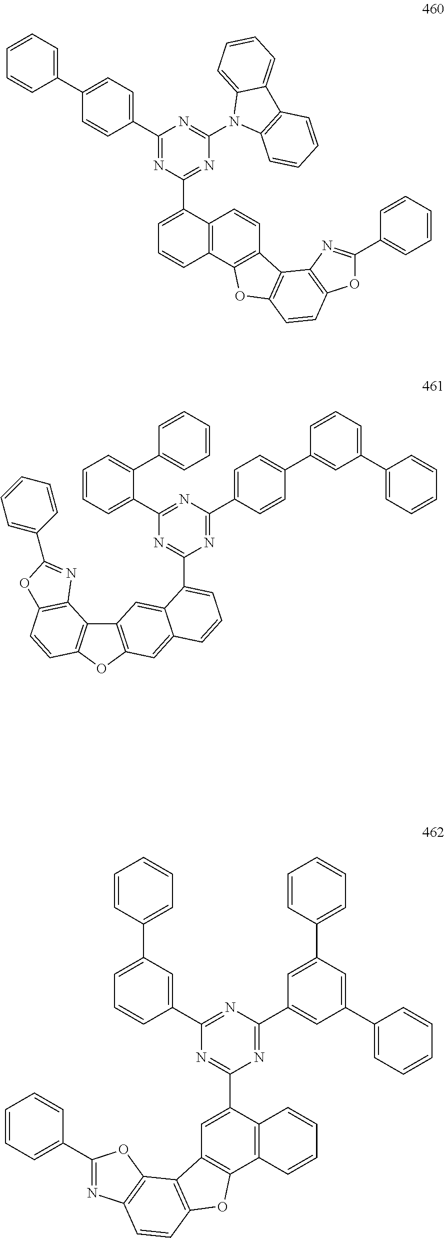 [SMILES: c1ccc(-c2ccc(-c3nc(-c4cccc5c4ccc4c5oc5ccc6oc(-c7ccccc7)nc6c54)nc(-n4c5ccccc5c5ccccc54)n3)cc2)cc1 ; c1ccc(-c2cccc(-c3ccc(-c4nc(-c5ccccc5-c5ccccc5)nc(-c5cccc6cc7oc8ccc9oc(-c%10ccccc%10)nc9c8c7cc56)n4)cc3)c2)cc1 ; c1ccc(-c2cccc(-c3nc(-c4cc(-c5ccccc5)cc(-c5ccccc5)c4)nc(-c4cc5c(oc6ccc7nc(-c8ccccc8)oc7c65)c5ccccc45)n3)c2)cc1]

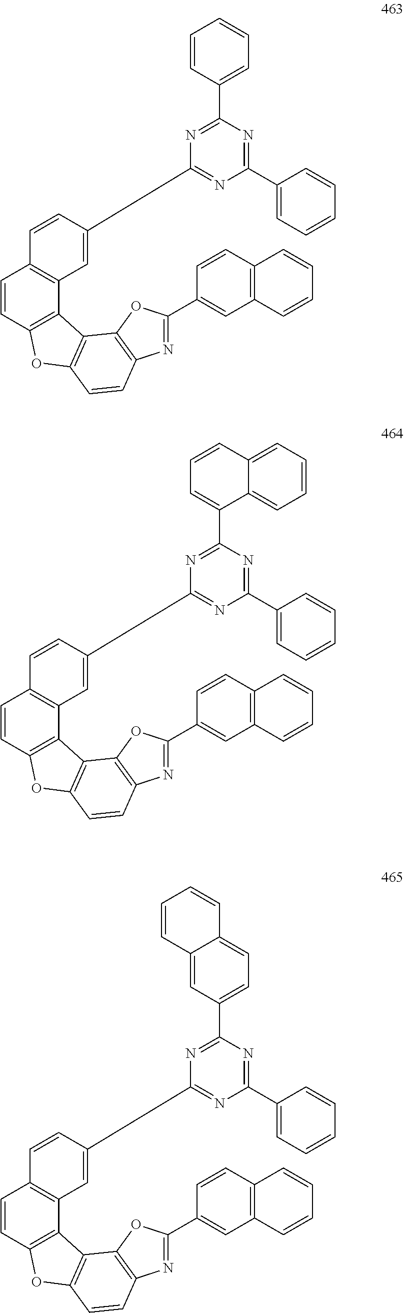 [SMILES: c1ccc(-c2nc(-c3ccccc3)nc(-c3ccc4ccc5oc6ccc7nc(-c8ccc9ccccc9c8)oc7c6c5c4c3)n2)cc1 ; c1ccc(-c2nc(-c3ccc4ccc5oc6ccc7nc(-c8ccc9ccccc9c8)oc7c6c5c4c3)nc(-c3cccc4ccccc34)n2)cc1 ; c1ccc(-c2nc(-c3ccc4ccccc4c3)nc(-c3ccc4ccc5oc6ccc7nc(-c8ccc9ccccc9c8)oc7c6c5c4c3)n2)cc1]

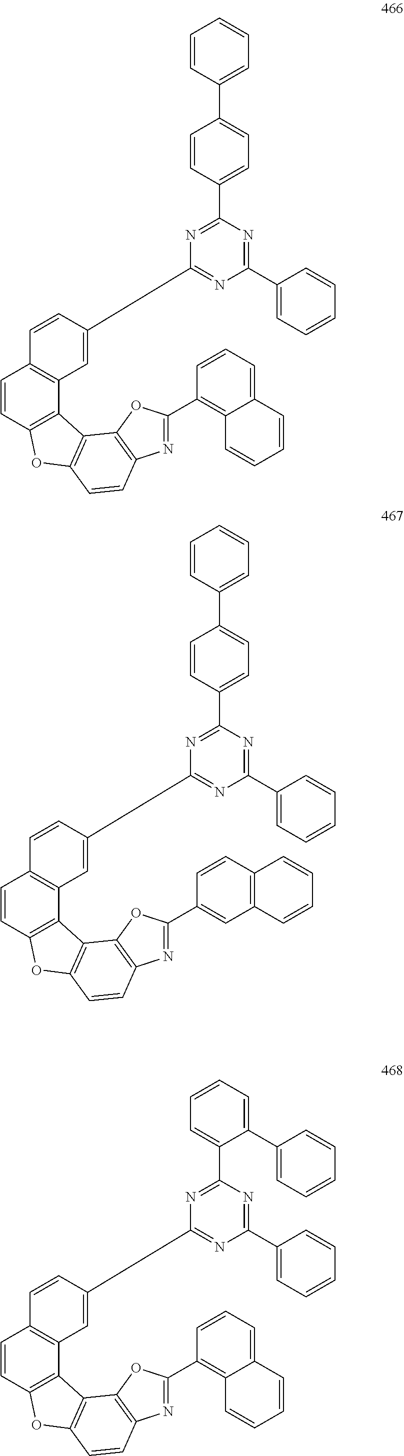 [SMILES: c1ccc(-c2ccc(-c3nc(-c4ccccc4)nc(-c4ccc5ccc6oc7ccc8nc(-c9cccc%10ccccc9%10)oc8c7c6c5c4)n3)cc2)cc1 ; c1ccc(-c2ccc(-c3nc(-c4ccccc4)nc(-c4ccc5ccc6oc7ccc8nc(-c9ccc%10ccccc%10c9)oc8c7c6c5c4)n3)cc2)cc1 ; c1ccc(-c2nc(-c3ccc4ccc5oc6ccc7nc(-c8cccc9ccccc89)oc7c6c5c4c3)nc(-c3ccccc3-c3ccccc3)n2)cc1]

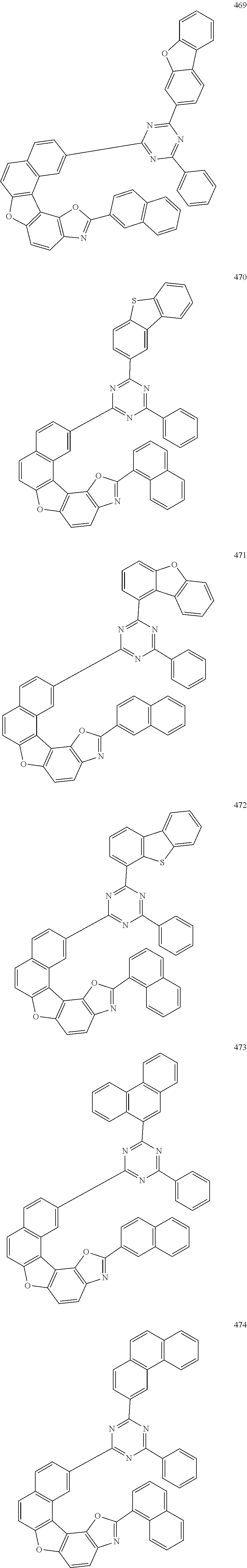

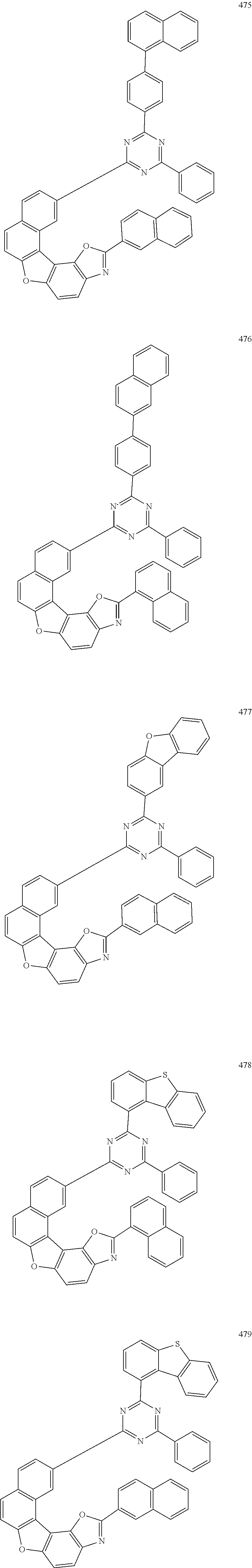

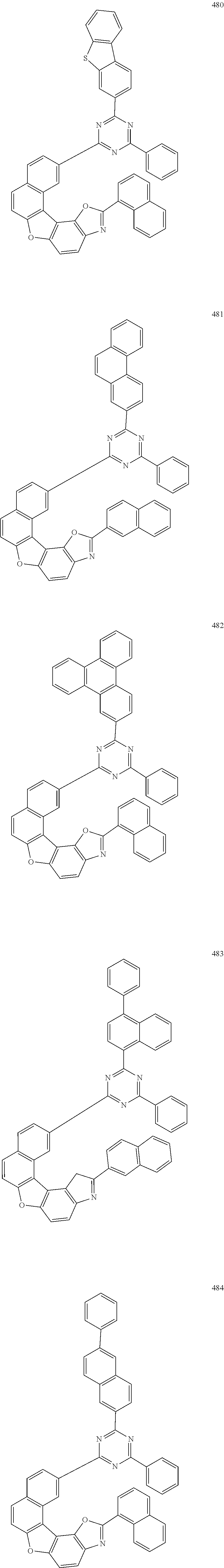

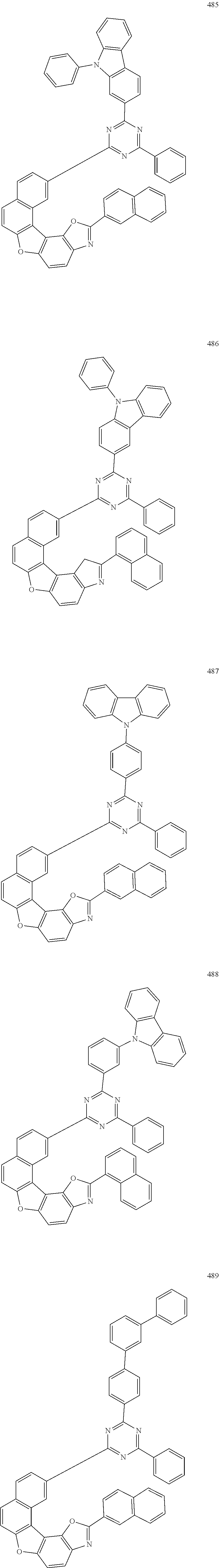

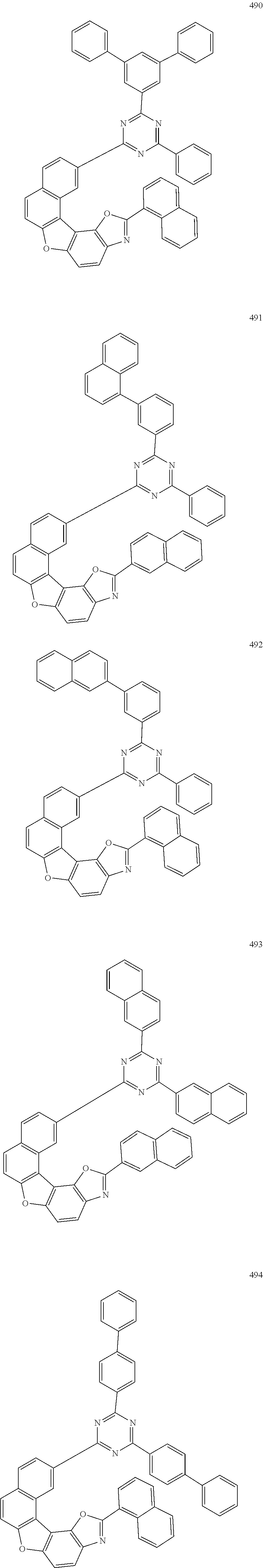

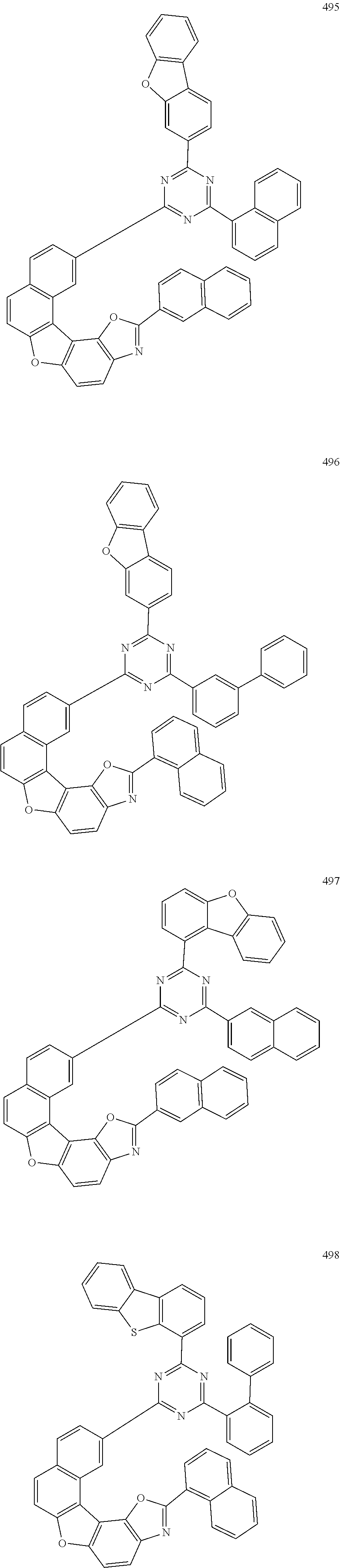 [SMILES: c1ccc2cc(-c3nc4ccc5oc6ccc7ccc(-c8nc(-c9ccc%10c(c9)oc9ccccc9%10)nc(-c9cccc%10ccccc9%10)n8)cc7c6c5c4o3)ccc2c1 ; c1ccc(-c2cccc(-c3nc(-c4ccc5c(c4)oc4ccccc45)nc(-c4ccc5ccc6oc7ccc8nc(-c9cccc%10ccccc9%10)oc8c7c6c5c4)n3)c2)cc1 ; c1ccc2cc(-c3nc(-c4ccc5ccc6oc7ccc8nc(-c9ccc%10ccccc%10c9)oc8c7c6c5c4)nc(-c4cccc5oc6ccccc6c45)n3)ccc2c1 ; c1ccc(-c2ccccc2-c2nc(-c3ccc4ccc5oc6ccc7nc(-c8cccc9ccccc89)oc7c6c5c4c3)nc(-c3cccc4c3sc3ccccc34)n2)cc1]

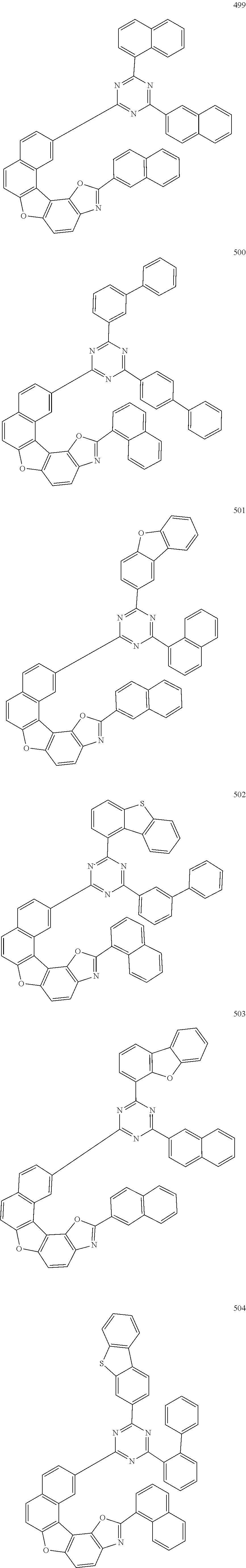

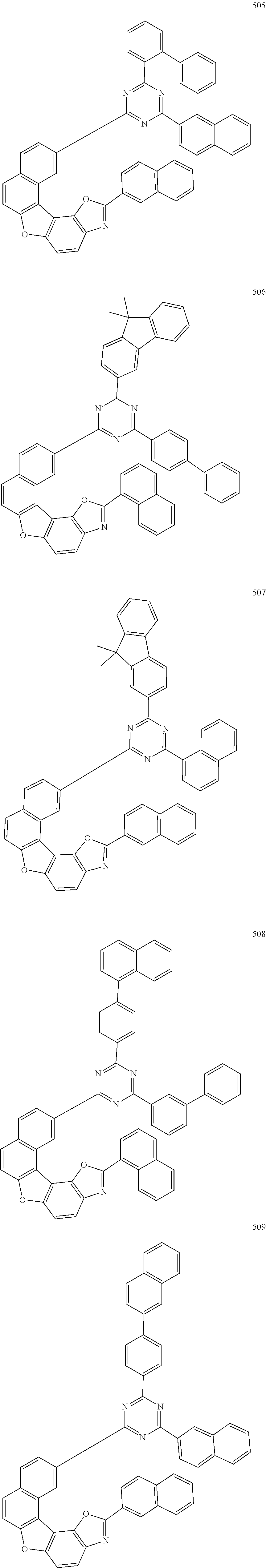

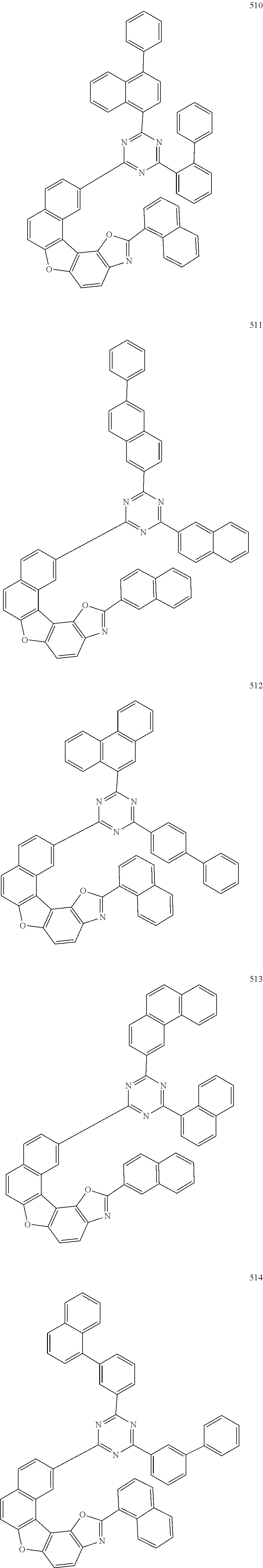

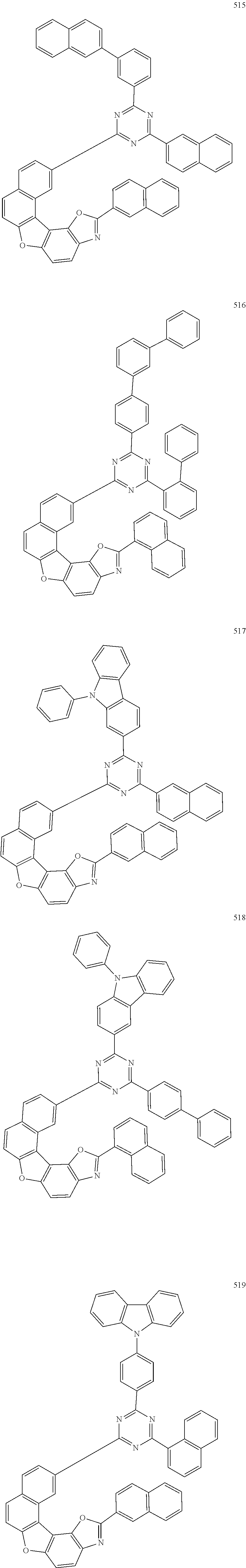

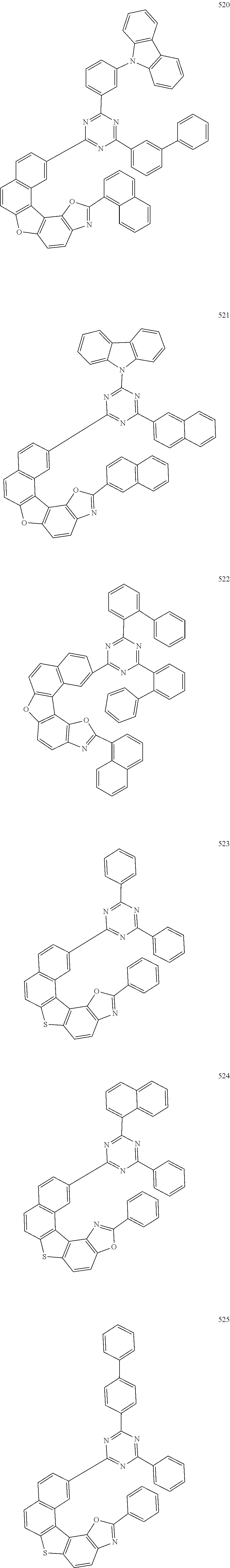

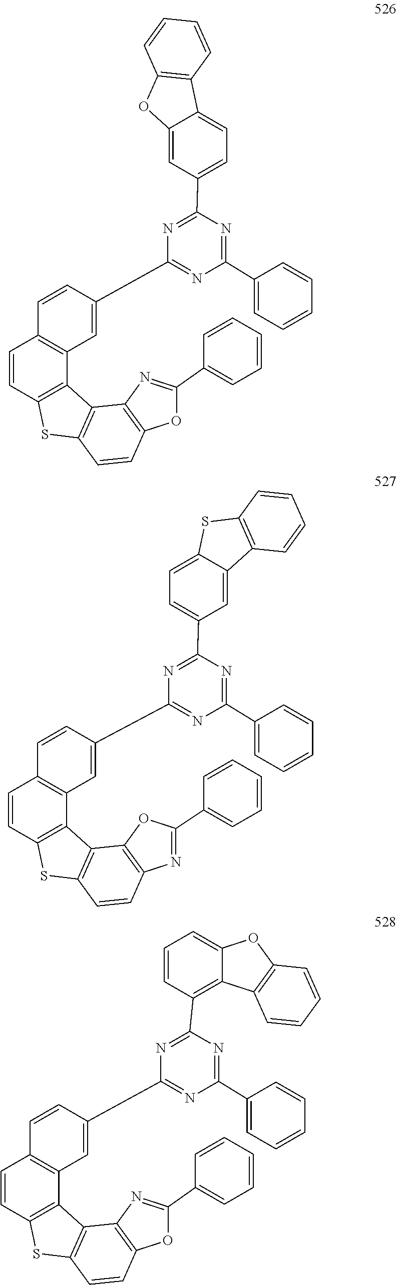 [SMILES: c1ccc(-c2nc(-c3ccc4c(c3)oc3ccccc34)nc(-c3ccc4ccc5sc6ccc7oc(-c8ccccc8)nc7c6c5c4c3)n2)cc1 ; c1ccc(-c2nc(-c3ccc4sc5ccccc5c4c3)nc(-c3ccc4ccc5sc6ccc7nc(-c8ccccc8)oc7c6c5c4c3)n2)cc1 ; c1ccc(-c2nc(-c3ccc4ccc5sc6ccc7oc(-c8ccccc8)nc7c6c5c4c3)nc(-c3cccc4oc5ccccc5c34)n2)cc1]

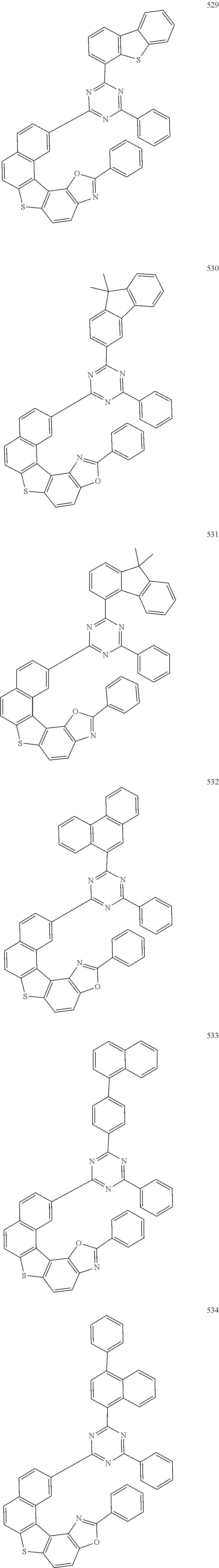

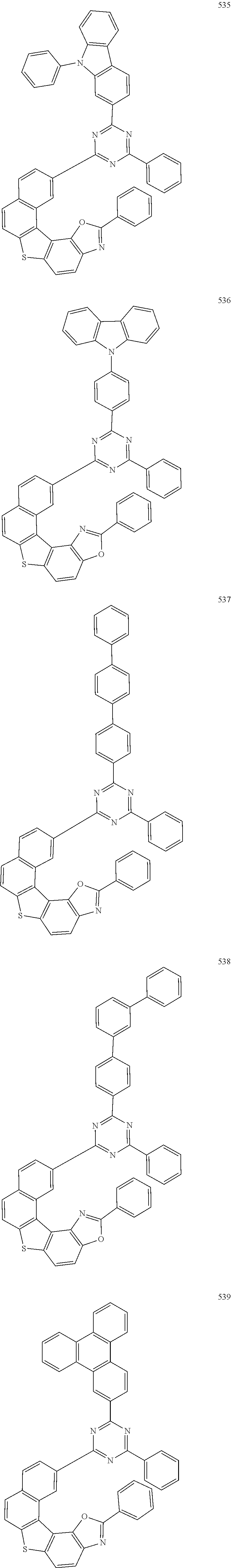 [SMILES: c1ccc(-c2nc(-c3ccc4ccc5sc6ccc7nc(-c8ccccc8)oc7c6c5c4c3)nc(-c3ccc4c5ccccc5n(-c5ccccc5)c4c3)n2)cc1 ; c1ccc(-c2nc(-c3ccc(-n4c5ccccc5c5ccccc54)cc3)nc(-c3ccc4ccc5sc6ccc7oc(-c8ccccc8)nc7c6c5c4c3)n2)cc1 ; c1ccc(-c2ccc(-c3ccc(-c4nc(-c5ccccc5)nc(-c5ccc6ccc7sc8ccc9nc(-c%10ccccc%10)oc9c8c7c6c5)n4)cc3)cc2)cc1 ; c1ccc(-c2cccc(-c3ccc(-c4nc(-c5ccccc5)nc(-c5ccc6ccc7sc8ccc9oc(-c%10ccccc%10)nc9c8c7c6c5)n4)cc3)c2)cc1 ; c1ccc(-c2nc(-c3ccc4c5ccccc5c5ccccc5c4c3)nc(-c3ccc4ccc5sc6ccc7nc(-c8ccccc8)oc7c6c5c4c3)n2)cc1]

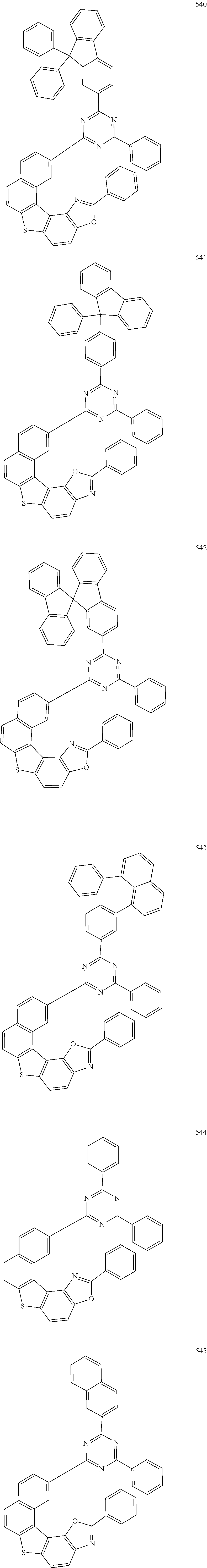

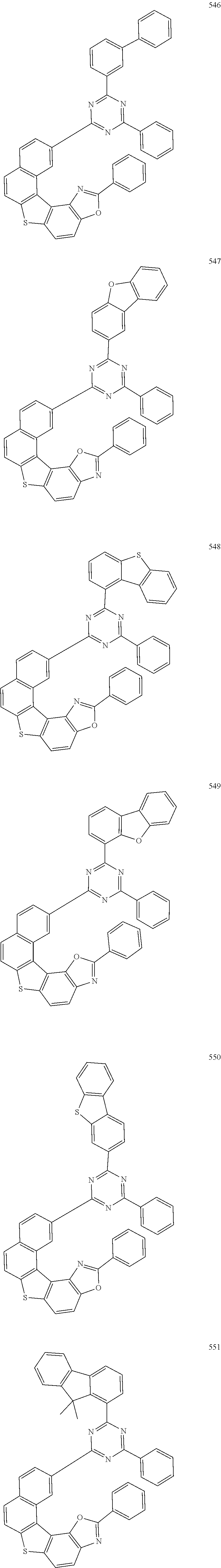

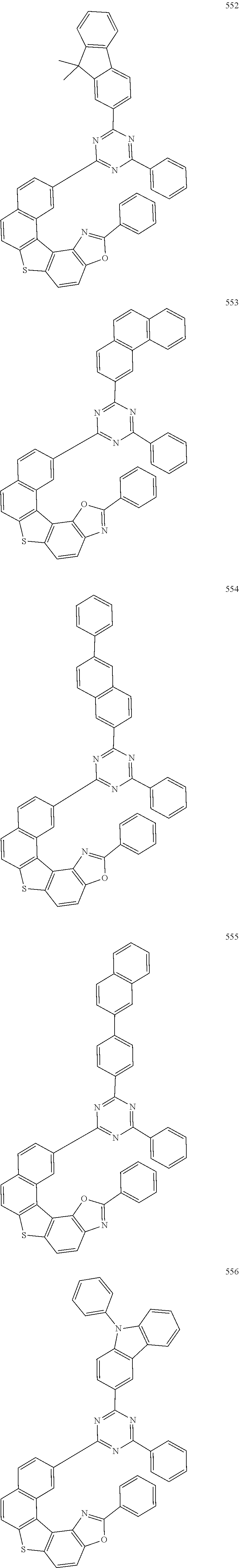 [SMILES: CC1(C)c2ccccc2-c2ccc(-c3nc(-c4ccccc4)nc(-c4ccc5ccc6sc7ccc8oc(-c9ccccc9)nc8c7c6c5c4)n3)cc21 ; c1ccc(-c2nc(-c3ccc4ccc5ccccc5c4c3)nc(-c3ccc4ccc5sc6ccc7nc(-c8ccccc8)oc7c6c5c4c3)n2)cc1 ; c1ccc(-c2ccc3cc(-c4nc(-c5ccccc5)nc(-c5ccc6ccc7sc8ccc9oc(-c%10ccccc%10)nc9c8c7c6c5)n4)ccc3c2)cc1 ; c1ccc(-c2nc(-c3ccc(-c4ccc5ccccc5c4)cc3)nc(-c3ccc4ccc5sc6ccc7nc(-c8ccccc8)oc7c6c5c4c3)n2)cc1 ; c1ccc(-c2nc(-c3ccc4ccc5sc6ccc7oc(-c8ccccc8)nc7c6c5c4c3)nc(-c3ccc4c(c3)c3ccccc3n4-c3ccccc3)n2)cc1]

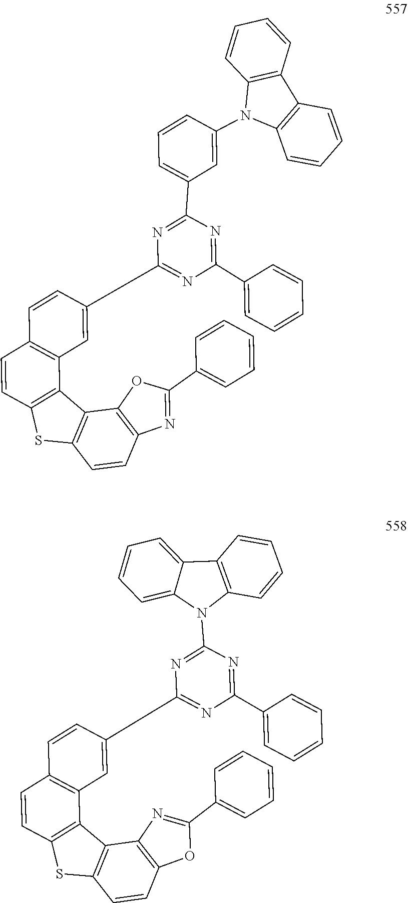 [SMILES: c1ccc(-c2nc(-c3cccc(-n4c5ccccc5c5ccccc54)c3)nc(-c3ccc4ccc5sc6ccc7nc(-c8ccccc8)oc7c6c5c4c3)n2)cc1 ; c1ccc(-c2nc(-c3ccc4ccc5sc6ccc7oc(-c8ccccc8)nc7c6c5c4c3)nc(-n3c4ccccc4c4ccccc43)n2)cc1]

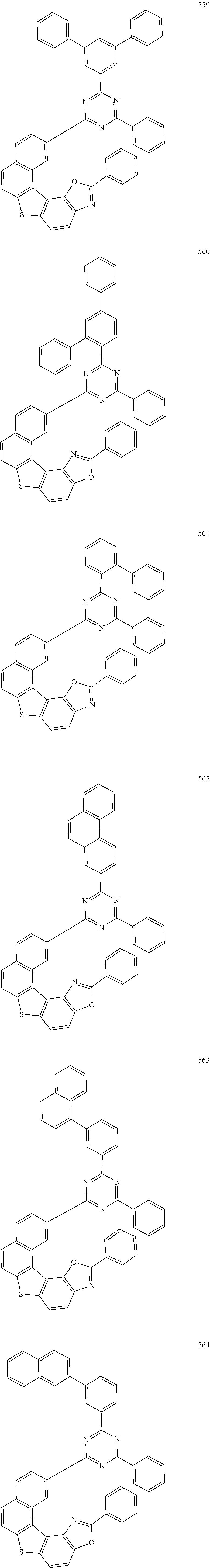

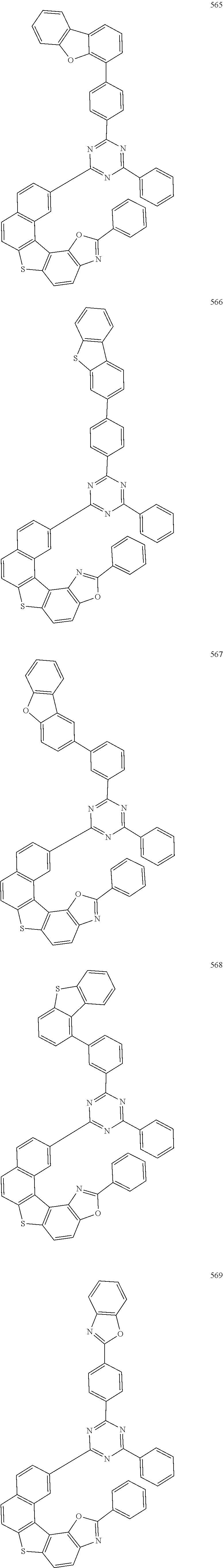 [SMILES: c1ccc(-c2nc(-c3ccc(-c4cccc5c4oc4ccccc45)cc3)nc(-c3ccc4ccc5sc6ccc7nc(-c8ccccc8)oc7c6c5c4c3)n2)cc1 ; c1ccc(-c2nc(-c3ccc(-c4ccc5c(c4)sc4ccccc45)cc3)nc(-c3ccc4ccc5sc6ccc7oc(-c8ccccc8)nc7c6c5c4c3)n2)cc1 ; c1ccc(-c2nc(-c3cccc(-c4ccc5oc6ccccc6c5c4)c3)nc(-c3ccc4ccc5sc6ccc7nc(-c8ccccc8)oc7c6c5c4c3)n2)cc1 ; c1ccc(-c2nc(-c3cccc(-c4cccc5sc6ccccc6c45)c3)nc(-c3ccc4ccc5sc6ccc7oc(-c8ccccc8)nc7c6c5c4c3)n2)cc1 ; c1ccc(-c2nc(-c3ccc(-c4nc5ccccc5o4)cc3)nc(-c3ccc4ccc5sc6ccc7nc(-c8ccccc8)oc7c6c5c4c3)n2)cc1]

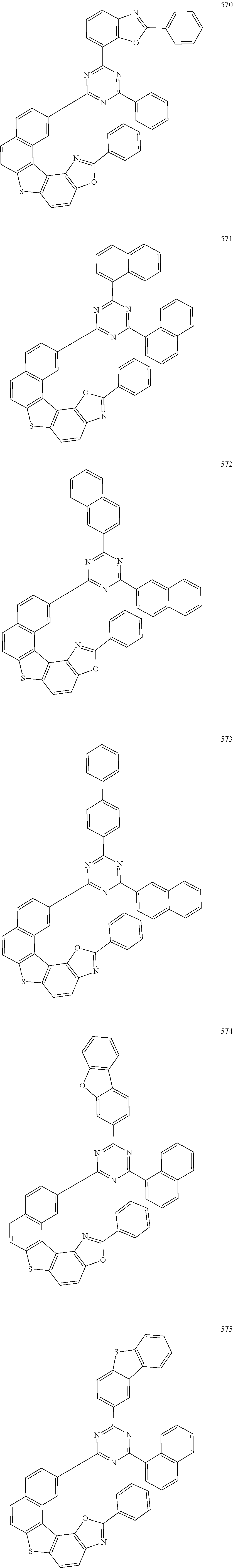

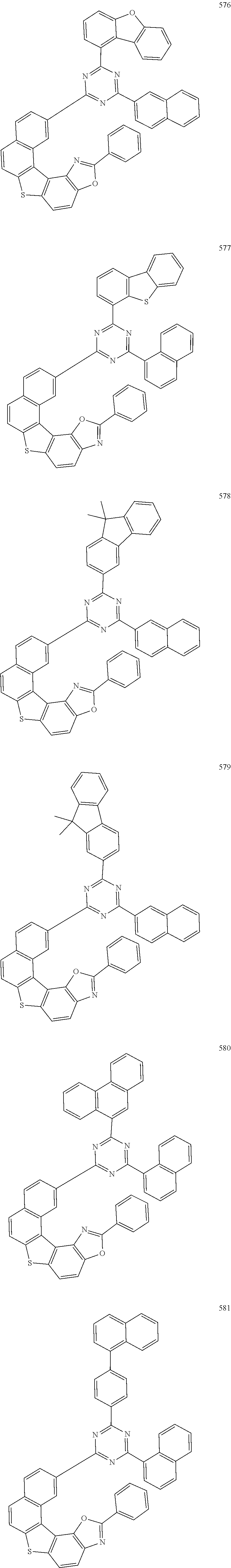

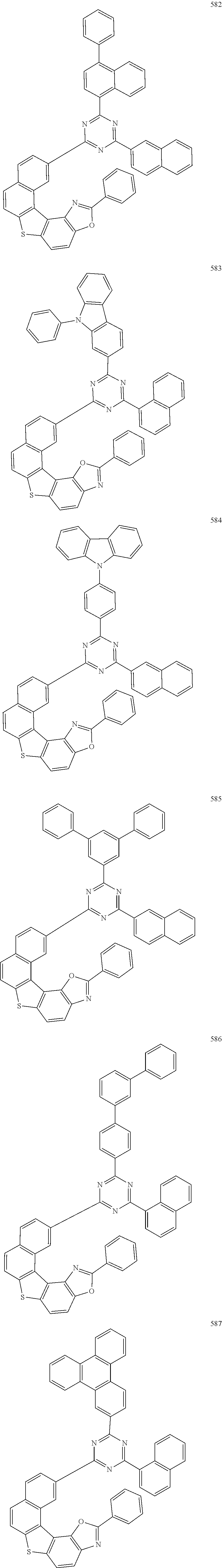

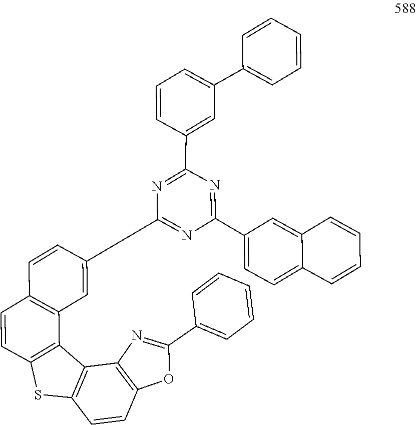 [SMILES: c1ccc(-c2cccc(-c3nc(-c4ccc5ccccc5c4)nc(-c4ccc5ccc6sc7ccc8oc(-c9ccccc9)nc8c7c6c5c4)n3)c2)cc1]

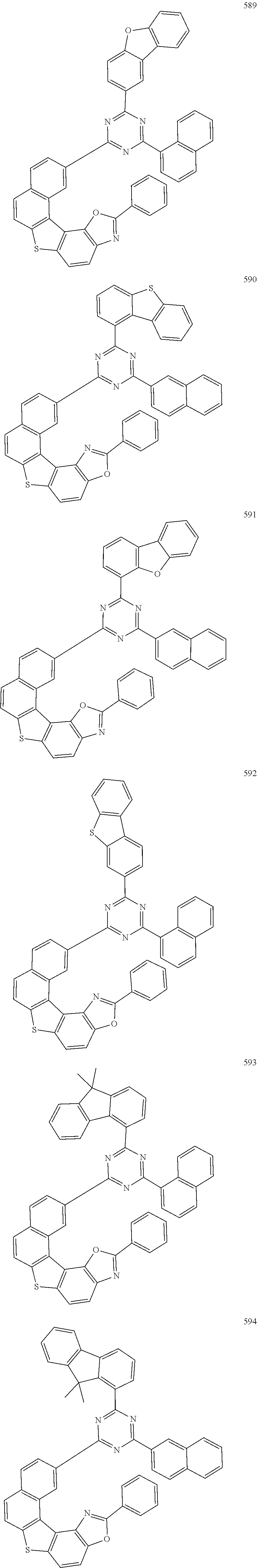

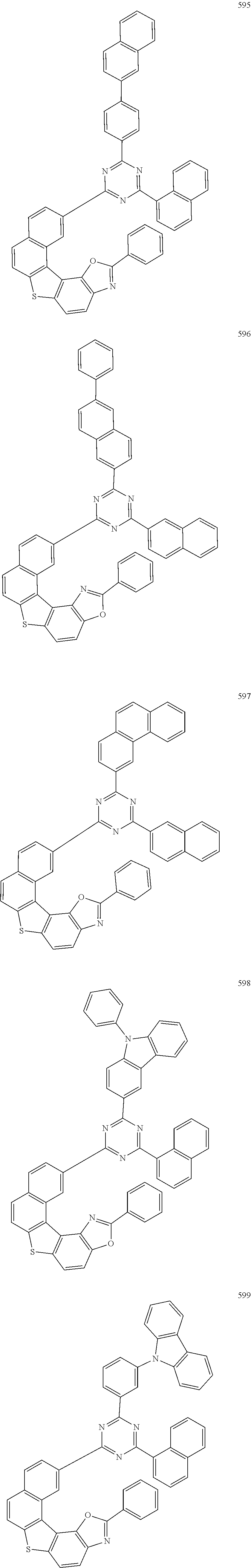

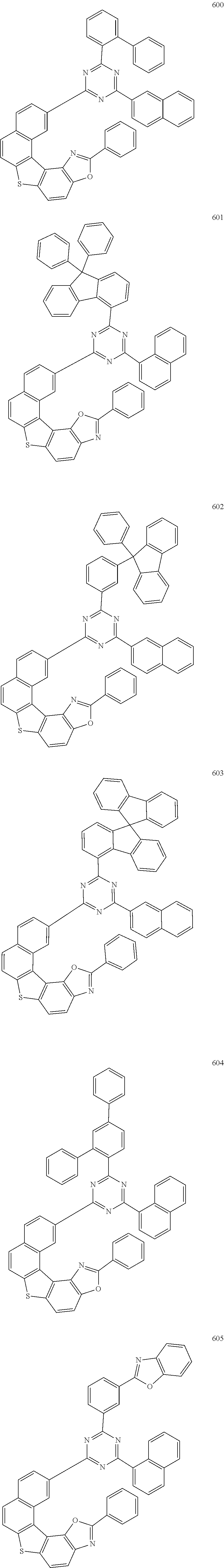

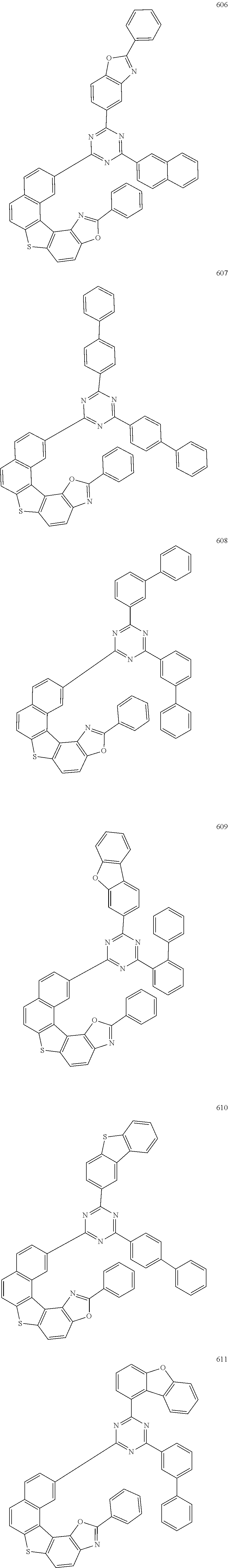

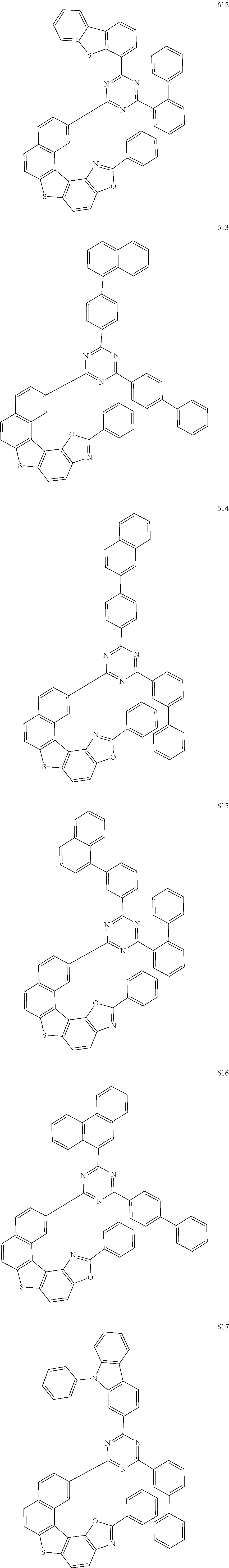

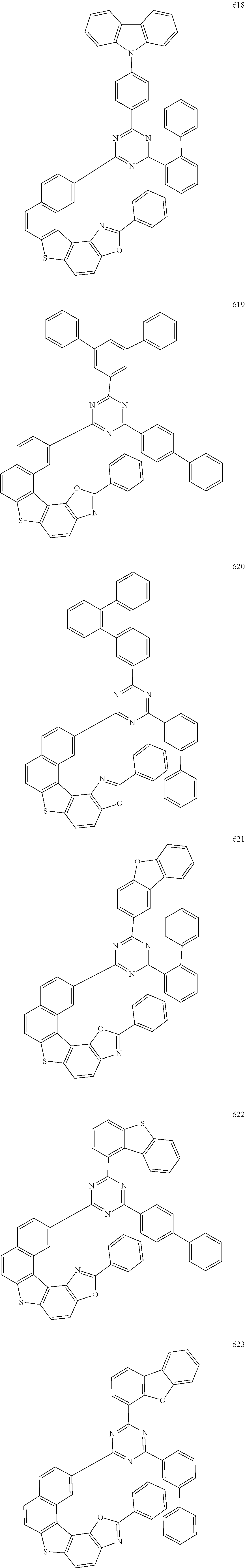

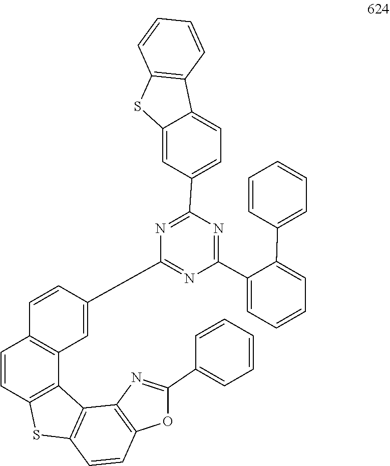 [SMILES: c1ccc(-c2nc3c(ccc4sc5ccc6ccc(-c7nc(-c8ccc9c(c8)sc8ccccc89)nc(-c8ccccc8-c8ccccc8)n7)cc6c5c43)o2)cc1]

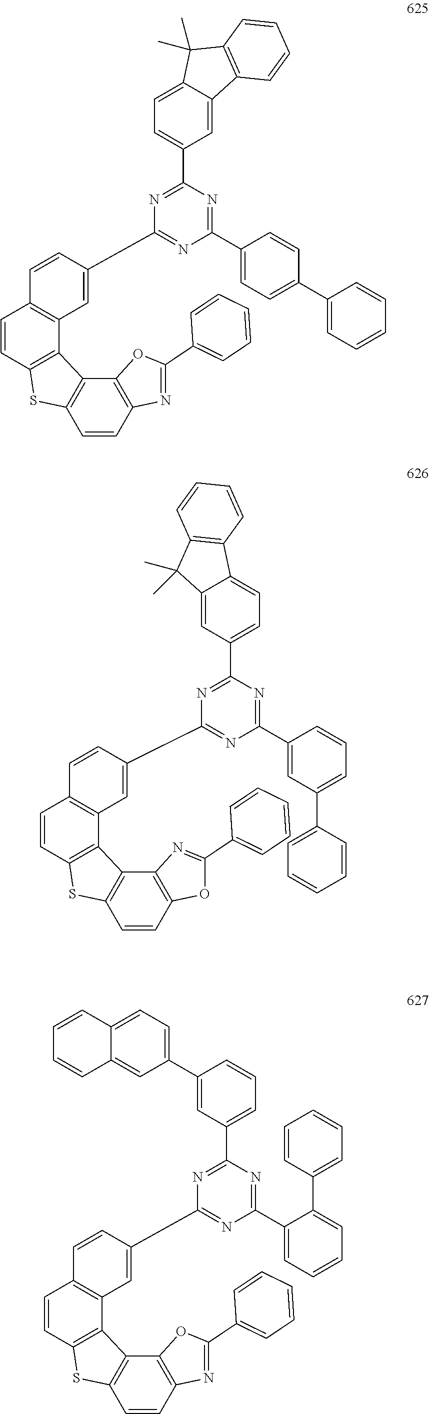 [SMILES: CC1(C)c2ccccc2-c2cc(-c3nc(-c4ccc(-c5ccccc5)cc4)nc(-c4ccc5ccc6sc7ccc8nc(-c9ccccc9)oc8c7c6c5c4)n3)ccc21 ; CC1(C)c2ccccc2-c2ccc(-c3nc(-c4cccc(-c5ccccc5)c4)nc(-c4ccc5ccc6sc7ccc8oc(-c9ccccc9)nc8c7c6c5c4)n3)cc21 ; c1ccc(-c2nc3ccc4sc5ccc6ccc(-c7nc(-c8cccc(-c9ccc%10ccccc%10c9)c8)nc(-c8ccccc8-c8ccccc8)n7)cc6c5c4c3o2)cc1]

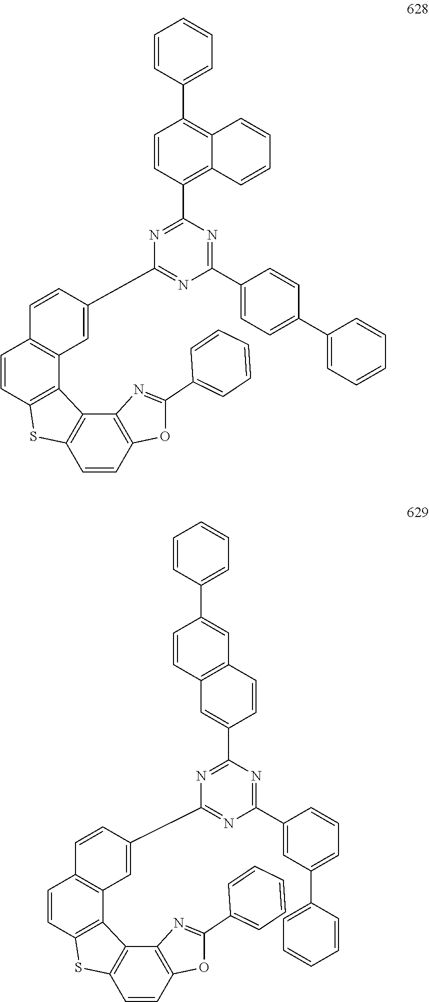 [SMILES: c1ccc(-c2ccc(-c3nc(-c4ccc5ccc6sc7ccc8oc(-c9ccccc9)nc8c7c6c5c4)nc(-c4ccc(-c5ccccc5)c5ccccc45)n3)cc2)cc1 ; c1ccc(-c2cccc(-c3nc(-c4ccc5cc(-c6ccccc6)ccc5c4)nc(-c4ccc5ccc6sc7ccc8oc(-c9ccccc9)nc8c7c6c5c4)n3)c2)cc1]

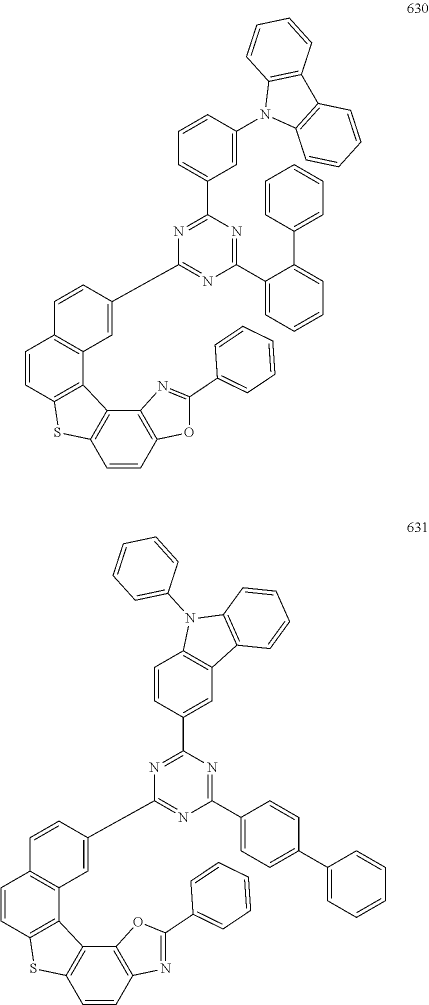 [SMILES: c1ccc(-c2nc3c(ccc4sc5ccc6ccc(-c7nc(-c8cccc(-n9c%10ccccc%10c%10ccccc%109)c8)nc(-c8ccccc8-c8ccccc8)n7)cc6c5c43)o2)cc1 ; c1ccc(-c2ccc(-c3nc(-c4ccc5ccc6sc7ccc8nc(-c9ccccc9)oc8c7c6c5c4)nc(-c4ccc5c(c4)c4ccccc4n5-c4ccccc4)n3)cc2)cc1]

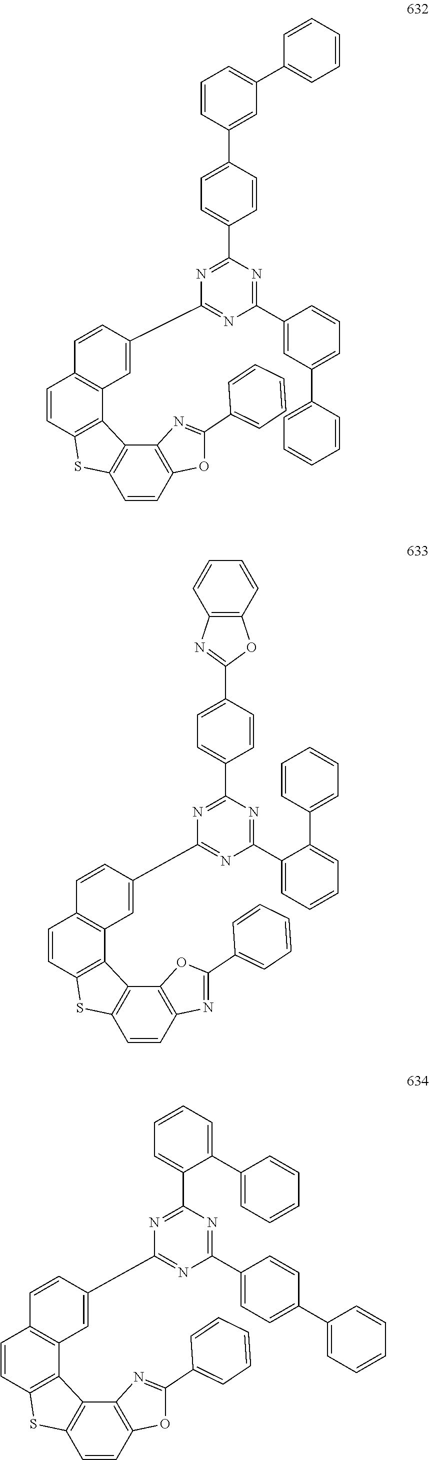 [SMILES: c1ccc(-c2cccc(-c3ccc(-c4nc(-c5cccc(-c6ccccc6)c5)nc(-c5ccc6ccc7sc8ccc9oc(-c%10ccccc%10)nc9c8c7c6c5)n4)cc3)c2)cc1 ; c1ccc(-c2nc3ccc4sc5ccc6ccc(-c7nc(-c8ccc(-c9nc%10ccccc%10o9)cc8)nc(-c8ccccc8-c8ccccc8)n7)cc6c5c4c3o2)cc1 ; c1ccc(-c2ccc(-c3nc(-c4ccc5ccc6sc7ccc8oc(-c9ccccc9)nc8c7c6c5c4)nc(-c4ccccc4-c4ccccc4)n3)cc2)cc1]

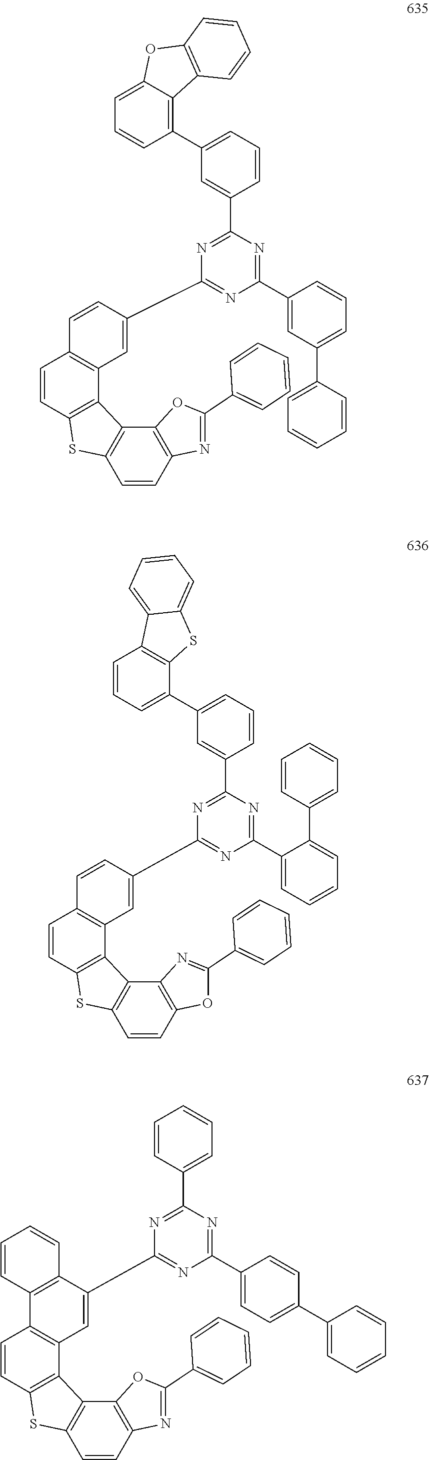 [SMILES: c1ccc(-c2cccc(-c3nc(-c4cccc(-c5cccc6oc7ccccc7c56)c4)nc(-c4ccc5ccc6sc7ccc8nc(-c9ccccc9)oc8c7c6c5c4)n3)c2)cc1 ; c1ccc(-c2nc3c(ccc4sc5ccc6ccc(-c7nc(-c8cccc(-c9cccc%10c9sc9ccccc9%10)c8)nc(-c8ccccc8-c8ccccc8)n7)cc6c5c43)o2)cc1 ; c1ccc(-c2ccc(-c3nc(-c4ccccc4)nc(-c4cc5c(ccc6sc7ccc8nc(-c9ccccc9)oc8c7c65)c5ccccc45)n3)cc2)cc1]

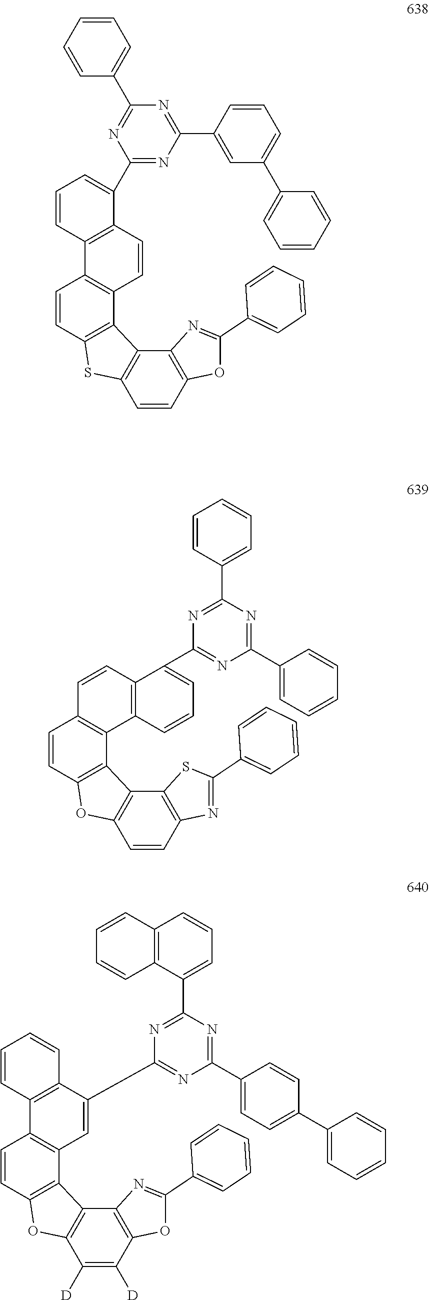 [SMILES: c1ccc(-c2cccc(-c3nc(-c4ccccc4)nc(-c4cccc5c4ccc4c5ccc5sc6ccc7oc(-c8ccccc8)nc7c6c54)n3)c2)cc1 ; c1ccc(-c2nc(-c3ccccc3)nc(-c3cccc4c3ccc3ccc5oc6ccc7nc(-c8ccccc8)sc7c6c5c34)n2)cc1 ; [2H]c1c([2H])c2oc3ccc4c5ccccc5c(-c5nc(-c6ccc(-c7ccccc7)cc6)nc(-c6cccc7ccccc67)n5)cc4c3c2c2nc(-c3ccccc3)oc12]

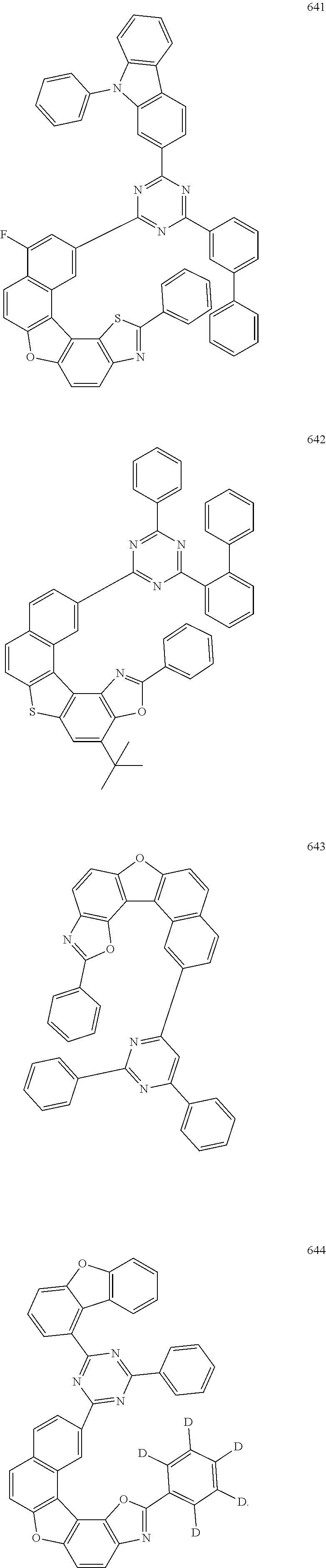

13. An organic electroluminescent device, comprising an anode and a cathode disposed opposite to each other, and a functional layer disposed between the anode and the cathode, wherein the functional layer comprises the nitrogen-containing compound according to claim 1;
optionally, the functional layer comprises an organic light emitting layer, the organic light emitting layer comprises the nitrogen-containing compound.

14. An electronic apparatus, comprising the organic electroluminescent device according to claim 13.
